(19) United States
(12) Patent Application Publication
(10) Pub. No.: US 2007/0105819 A1
(43) Pub. Date: May 10, 2007

(54) CB-1 modulating compounds and their use

(76) Inventors: Roger Olsson, Bunkeflostrand (SE); Fredrik Ek, Lund (SE); Lars K. Ottesen, Valby (DK); Anne Bulow, Copenhagen S. (DK)

Correspondence Address: KNOBBE MARTENS OLSON & BEAR LLP, 2040 MAIN STREET, FOURTEENTH FLOOR, IRVINE, CA, 92614, US

(21) Appl. No.: 11/583,141

(22) Filed: Oct. 17, 2006

Related U.S. Application Data
(60) Provisional application No. 60/727,997, filed on Oct. 17, 2005.
(60) Provisional application No. 60/831,003, filed on Jul. 14, 2006.
(60) Provisional application No. 60/832,510, filed on Jul. 21, 2006.

(51) Int. Cl. A61K 31/695; A61K 31/55; A61K 31/551; A61K 31/554; A61K 31/553

(52) U.S. Cl. 514/63; 514/211.11; 514/220; 514/217; 540/547; 540/557

(57) ABSTRACT

Disclosed herein is a compound of Formula (I). Also disclosed herein is a method of modulating the activity of a cannabinoid receptor using a compound of Formula (I). Furthermore, disclosed herein is a method of treating a disease or condition that would be alleviated, improved or prevented by administration of a compound that modulates a cannabinoid receptor comprising identifying a subject in need thereof and administering to said subject a therapeutically effective amount of a compound of Formula (I). Also disclosed herein are pharmaceutical compositions comprising a compound of Formula (I).

This application claims priority to U.S. Provisional Patent Application Ser. Nos. 60/727,997, entitled “CB-1 MODULATING COMPOUNDS AND THEIR USE”, filed Oct. 17, 2005; 60/831,003, entitled “CB-1 MODULATING COMPOUNDS AND THEIR USE”, filed Jul. 14, 2006; 60/832,510, entitled “CB-1 MODULATING COMPOUNDS AND THEIR USE”, filed Jul. 21, 2006; which are all incorporated by reference herein in their entireties, including any drawings.

### BACKGROUND OF THE INVENTION

1. Field of the Invention

This invention relates to the fields of organic chemistry, pharmaceutical chemistry, biochemistry, molecular biology and medicine. In particular it relates to compounds that modulate the activity of the human cannabinoid receptor (CB1), and to the use of the compounds for the treatment and prevention of diseases and disorders related to CB1.

2. Description of the Related Art

The cannabinoids, which are bioactive lipids, naturally found in the cannabis sativa (marijuana) plant, have been used recreationally and therapeutically for at least 5000 years. In addition to their well-documented effects on mood, cannabinoids (often in the form of marijuana) have been prescribed to treat nausea, pain, migraine, epilepsy, glaucoma, hypertension, cachexia and pain associated with childbirth. Two cannabinoid receptors, CB1 and CB2, have been identified. Both are members of the G protein-coupled receptor superfamily, and are negatively coupled through Gi protein. The CB2 receptor has 44% sequence similarity to the CB1 receptor.

The CB1 receptor, unlike the CB2 receptor, is highly expressed in the central nervous system, mostly presynaptically. Indeed, the CB1 receptor is present in the brain at higher levels than many other GPCRs. It is found in the cortex, cerebellum, hippocampus, and basal ganglia (reviewed in Brievogel and Childres, 1998). In addition, the CB1 receptor has also been detected in sperm, the prostate gland, and other peripheral tissues (including structures of the eye). The CB2 receptor is present in the cells of the immune system (spleen, thymus), testis, and lung.

The CB1 receptor is believed to be responsible for the appetite stimulating properties and habituation associated with cannabinoid use. The CB1 receptor antagonist, SR141716 (rimonabant, Acomplia; Sanofi-Aventis) has shown efficacy in late-stage clinical trials for obesity and nicotine dependence, with no psychotropic effects. The compound has been shown to reduce both food intake and adipose tissue (by a mechanism independent of food intake). Use of SR141716 in animal models suggests additional use of CB1 receptor antagonists and inverse agonists for the treatment of alcohol addiction, opiate addiction, cocaine addiction, anxiety, and septic shock. Interestingly, mice null for the CB1 gene also display impaired cocaine self-administration, and less severe withdrawal from morphine addiction compared to wild-type mice. In addition, CB1 knockout mice also display increased bone mineral density, and both CB1 knockout mice and mice treated with CB antagonists are resistant to bone loss in a model for osteoporosis. Other animal models indicate a use for CB1 receptor antagonists and inverse agonists for the prevention of premature spontaneous abortion.

Cannabinoid signaling is hyperactive in animal models of several diseases suggesting that cannabinoids either have a protective role (e.g., CB1 agonists may be therapeutic) or are involved in the pathology of these diseases (e.g., CB1 antagonists or inverse agonists may be therapeutic). These include Parkinson's disease, Alzheimer's disease, multiple sclerosis, epilepsy, and intestinal disorders. In addition, the levels of endogenous cannabinoids and CB1 receptors are elevated in the liver and blood of patients with cirrhosis of the liver. Moreover, cannabinoid levels have been shown to be elevated in the cerebrospinal fluid of a patient with stroke, as well as in the brains of depressed suicide victims. Endogenous cannabinoids have also been shown to be higher in the cerebrospinal fluid of drug-naive paranoid schizophrenics compared to normal patients; interestingly, schizophrenic patients treated with atypical but not typical antipsychotics also exhibit higher CSF levels of anandamide. Additionally, the CB1 gene is located in a chromosomal region that has been linked to schizophrenia. Moreover, high levels of the endogenous cannabinoid, anandamide, are correlated with premature abortion and failure of in vitro fertilization. Finally, activation of CB receptors by an anandamide analogue has been shown to reduce sperm fertilizing capacity by 50%.

Selective activation of CB1 receptors by agonists or partial agonists may also be used to treat a number of disorders. Some patients in clinical trials of the CB1 antagonist, SR141716A, have reported diarrhea and nausea, suggesting that an agonist would alleviate those symptoms. THC (tetrahydrocannabinol; active cannabinoid in Cannabis sativa) has been shown to improve mobility and alleviate pain in patients with multiple sclerosis. Other promising results for cannabinoids have been shown in clinical trials for Tourette's syndrome, Parkinson's disease, glaucoma, and pain. Finally cannabinoids have been shown to inhibit cancer growth, angiogenesis, and metastasis in animal models.

### SUMMARY OF THE INVENTION

Disclosed herein is a compound of Formula (I):

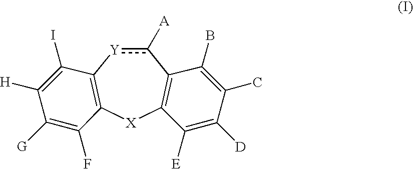

Also disclosed herein is a method of modulating the activity of a cannabinoid receptor using a compound of Formula (I). Furthermore, disclosed herein is a method of treating a disease and/or condition that would be alleviated, improved, and/or prevented by administration of a compound that modulates a cannabinoid receptor comprising administering to a therapeutically effective amount of a compound of Formula (I). Also disclosed herein are pharmaceutical compositions comprising a compound of Formula (I).

### BRIEF DESCRIPTION OF THE DRAWINGS

FIG. 1A is a graph showing the percent response of the CB1 receptor as the concentration of 11-Cyclohexyl-dibenzo[b,f] [1,4]thiazepine-8-carboxylic acid piperidin-1-ylamide (Compound I) increases. FIG. 1B is a graph showing the percent response of the CB2 receptor as the concentration of Compound I increase.

FIG. 2 is a bar graph showing the food intake in fasted rats 1 and 2 hours after being administered either 1, 3, or 10 mg/kg doses of Compound I. * Indicates p<0.05 as compared to the vehicle-treated controls. ** Indicates p<0.01 as compared to the vehicle-treated controls.

FIG. 3 is bar graph showing the time course food intake in fasted rats after being administered 1 mg/kg of Compound I. * Indicates p<0.05 as compared to the vehicle-treated controls. ** Indicates p<0.01 as compared to the vehicle-treated controls.

FIG. 4 is a bar graph showing cumulative food consumption at several points in time after the rats had been dosed with 10 mg/kg of Compound I. * Indicates p<0.05 as compared to the vehicle-treated controls.

FIG. 5A is a line graph showing the attenuation of CB1 agonist-mediated effects after administration of CP 55,940 (0.3 and 1.0 mg/kg). FIG. 5B is a line graph showing the attenuation of CB1 agonist-mediated effects after administration of Compound I alone or in combination with CP55,940.

FIG. 6 is a bar graph showing the body temperature of the rats at several points in time after the rats had been dosed with various doses of CP 55,950 or CP55,950 and Compound I.

FIG. 7 is a bar graph showing the concentration of Compound I in the plasma and brain at several points in time.

FIGS. 8A and 8B are bar graphs showing the concentration of compound, N-(butyl)-11-(4-chlorophenyl)-dibenzo[b,f,] [1,4]thiazepine-8-carboxamide (Compound II) in tissue and brain at several points in time. FIGS. 8C and 8D are line graphs showing the concentration of Compound II in the plasma and brain at several points in time.

FIG. 9A in a line graph showing the effects of Compound II (1 and 3 mg/kg/day) on body weight FIG. 9B is a line graph showing the effects of Compound II (1 and 3 mg/kg/day) on food intake and water intake. FIG. 9C line graph showing the effects of Compound II (10 mg/kg/day) on body weight. FIG. 9D is a line graph showing the effects of Compound II (10 mg/kg/day) on food intake and water intake.

FIGS. 10A and 10C are bar graphs showing the exploration ratio at 1 and 2 hours after the mice had been dosed with the vehicle, CP 55,940 (0.3 mg/kg, ip), or SR141716A (1 mg/kg, ip). FIGS. 10B and 10D are bar graphs showing the discrimination index at 1 and 2 hours after the mice had been dosed with the vehicle, CP 55,940 (0.3 mg/kg, ip), or SR141716A (1 mg/kg, ip).

FIG. 11A is a bar graph showing the exploration ratio 2 hours after the mice had been dosed with Compound II (3 mg/kg, ip). FIG. 11B is a bar graph showing the discrimination index 2 hours after the mice had been dosed with Compound II (3 mg/kg, ip).

FIG. 12 is a bar graph showing percentage of novel recognition of a familiar object 2 hours after the mice had been dosed with 1, 3, or 10 mg/kg of Compound II.

FIG. 13 is a line graph showing the working memory errors of the mice after being dosed with the vehicle, tacrine (0.3 mg/kg), or Compound II (3 mg/kg).

FIG. 14 is a line graph showing the contralateral rotations over time of the mice after being dosed with apomorphine (0.05, 0.16, and 0.5 mg/kg).

FIG. 15 is a line graph showing the contralateral rotations over time of the mice after being dosed with apomorphine (0.05 mg/kg), Compound II (3.0 mg/kg), or apomorphine (0.05 mg/kg) and Compound II (3.0 mg/kg).

FIG. 16 is a line graph showing the contralateral rotations over time of the mice after being dosed with apomorphine (0.16 mg/kg), Compound II (3.0 mg/kg), or apomorphine (0.16 mg/kg) and Compound II (3.0 mg/kg).

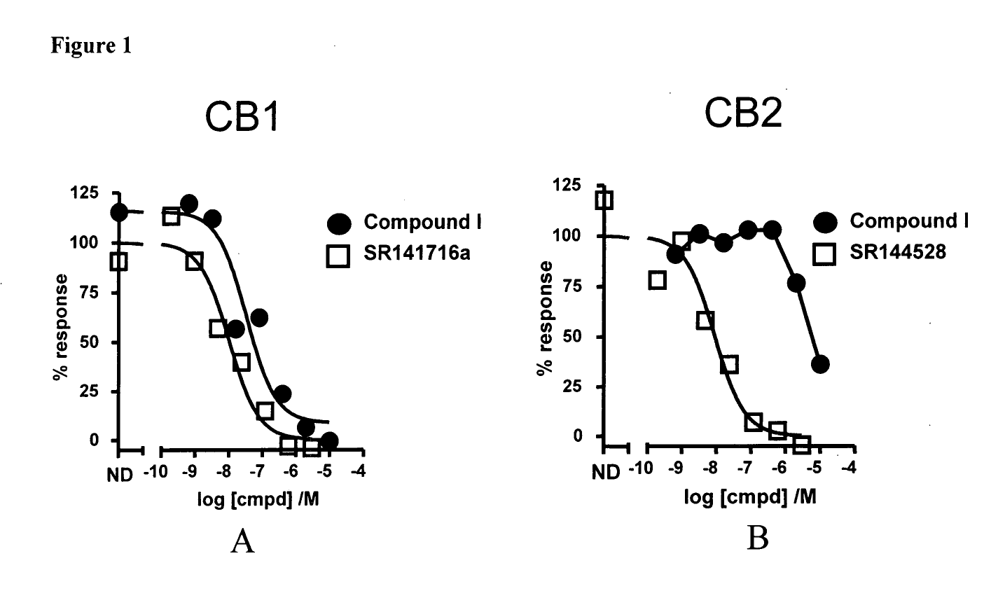

### DETAILED DESCRIPTION OF THE PREFERRED EMBODIMENTS

One embodiment described herein relates to a compound of formula (I):

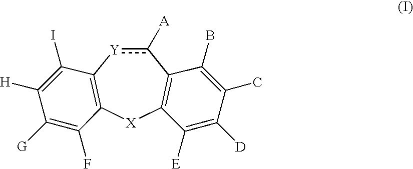

as a single isomer, a mixture of isomers, a racemic mixture of isomers, pharmaceutically acceptable salt, a solvate, metabolite or polymorph thereof, wherein:

X can be selected from the group consisting of O, S, S═O, SO₂, NR₁, NC≡N, NC(=Z)R₁, NC(=Z)NR₁ₐR_{1b}, CR₁ₐR_{1b}, C═O, C═CR₁ₐR_{1b}, and SiR₁ₐR_{1b};

Y can be —N(R₂) [symbol] or —C(R₁R₂) [symbol] ;

the symbol [symbol] represents a single or double bond, where when [symbol] is a double bond, R₂ is absent;

A can be selected from the group consisting of C₃-C₁₂alkyl, C₄-C₁₂alkyl, alkenyl, alkynyl, cycloalkyl, cycloalkenyl, cycloalkynyl, (cycloalkyl)alkyl, (cycloalkenyl)alkyl, (cycloalkynyl)alkyl, aryl, heteroaryl, heteroalicyclyl, aralkyl, heteroaralkyl, (heteroalicyclyl)alkyl, halogen, —NR₁ₐR_{1b}, —N═CR₁ₐR_{1b}, sulfenyl, sulfinyl, sulfonyl, and —(CH₂)₀₋₄—C(=Z)-OR₁, wherein any member of said group can be substituted or unsubstituted;

B, C, D, E, F, G and I can be separately selected from the group consisting of hydrogen, alkyl, alkenyl, alkynyl, cycloalkyl, cycloalkenyl, cycloalkynyl, aryl, heteroaryl, aralkyl, heteroaralkyl, heteroalicyclyl, (heteroalicyclyl)alkyl, halogen, hydroxyl, nitro, sulfenyl, sulfinyl, sulfonyl, haloalkyl, haloalkoxy, —CN, —C(=Z)R₁, —C(=Z)OR₁, —C(=Z)NR₁ₐR_{1b}, —C(=Z)N(R₁)NR₁ₐR_{b}, —C(=Z)N(R₁)N(R₁)C(=Z)R₁, —C(R₁)═NR₁, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁, wherein any member of said group can be substituted or unsubstituted except for hydrogen;

H can be selected from the group consisting of alkyl, alkenyl, alkynyl, cycloalkyl, cycloalkenyl, cycloalkynyl, aryl, heteroaryl, aralkyl, heteroaralkyl, heteroalicyclyl, (heteroalicyclyl)alkyl, halogen, hydroxyl, nitro, sulfenyl, sulfinyl, sulfonyl, haloalkyl, haloalkoxy, —CN, —C(=Z)R₁, —C(=Z)OR₁, —C(=Z)NR₁ₐR_{1b}, —C(=Z)N(R₁)NR₁ₐR_{1b}, —C(=Z)N(R₁)N(R₁)C(=Z)R₁, —C(R₁)═NR₁, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁, wherein any member of said group can be substituted or unsubstituted;

Z can be O (oxygen) or S (sulfur);

R₁, R₁ₐ and R_{1b} can each independently selected from the group consisting of: hydrogen, halogen, alkyl, alkenyl, alkynyl, cycloalkyl, cycloalkenyl, cycloalkynyl, aryl, aralkyl, heteroaryl, heteroaralkyl, heteroalicyclyl, (heteroalicyclyl)alkyl, —(CH₂)₀₋₇—OR₃, —(CH₂)₀₋₇—SR₃, —(CH₂)₀₋₇—NR₃ₐR_{3b}, haloalkyl, —C(=Z)R₃, —C(=Z)OR₃, and —C(=Z)NR₃ₐR_{3b}. wherein any member of said group can be substituted or unsubstituted except for hydrogen; or R₁ₐ and R_{1b} can be taken together to form an unsubstituted or substituted heteroalicyclyl having 2 to 9 carbon atoms or an unsubstituted or substituted carbocyclyl having 3 to 9 carbon atoms;

R₂ can be absent or is selected from the group consisting of: hydrogen, alkyl, alkenyl, alkynyl, cycloalkyl, cycloalkenyl, cycloalkynyl, aryl, heteroaryl, and heteroalicyclyl, wherein any member of said group can be substituted or unsubstituted except for hydrogen;

R₃, R₃ₐ, and R_{3b} can each independently selected from the group consisting of: hydrogen, alkyl, alkenyl, alkynyl, cycloalkyl, cycloalkenyl, aryl, heteroaryl, heteroalicyclyl, aralkyl, heteroaralkyl, and (heteroalicyclyl)alkyl, wherein any member of said group can be substituted or unsubstituted except for hydrogen;

In some embodiments, A cannot be a substituted or unsubstituted piperazine.

In other embodiments, H cannot be selected from the group consisting of —CF₃, phenyl, —OS(O)₂—CF₃, methyl, —CN, halogen, and 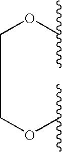
when A is a substituted or unsubstituted heteroalicyclyl containing at least one nitrogen or —NR₁ₐR_{1b}.

In still other embodiments, H cannot be halogen when A is substituted or unsubstituted aryl, substituted or unsubstituted aralkyl, substituted or unsubstituted heteroaryl, halogen, and substituted or unsubstituted sulfenyl; X is —NR₁, wherein R₁ is hydrogen; and Y is —N(R₂) [symbol] , wherein [symbol] is a double bond and R₂ is absent.

In yet still other embodiments, when X is O or —NR₁, wherein R₁ is methyl and Y is —N(R₂) [symbol] , wherein [symbol] is a double bond and R₂ is absent then H cannot be —C(=Z)OR₁, wherein R₁ is hydrogen, methyl, or ethyl.

In one embodiments, when A is halogen, Y is —N(R₂) [symbol] , wherein [symbol] is a double bond and R₂ is absent, and X is S then F cannot be —S(O)₂NR₁ₐR_{1b}, wherein R₁ₐ and R_{1b} are both hydrogen.

In one embodiments, the compound of Formula (I) can bind to a cannabinoid receptor. In certain embodiments, the cannabinoid receptor can be a CB1 receptor.

In some embodiments, R₁ₐ and R_{1b} can form an unsubstituted or substituted heteroalicyclyl having 2 to 9 carbon atoms and substituted with subtituents selected from the group consisting of hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino and protected amino. In other embodiments, R₁ₐ and R_{1b} can form an unsubstituted or substituted heteroalicyclyl having 2 to 9 carbon atoms selected from the group consisting of:

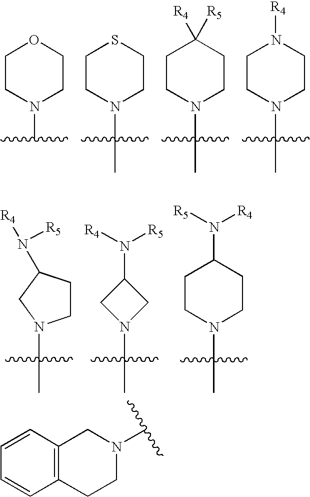

wherein R₄ and R₅ are each independently selected from the group consisting of hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxyl, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino and protected amino. In still other embodiments, R₁ₐ and R_{1b} can form an unsubstituted or substituted heteroalicyclyl having 2 to 9 carbon atoms selected from the group consisting of:

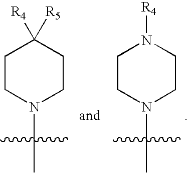

In some embodiments, R_{1b} can be hydrogen. In other embodiments, R_{1b} can be C₁₋₃alkyl.

In other embodiments, X can be S, SO, or SO₂.

In one embodiment, H can be selected from the group consisting of aryl, heteroaryl, aralkyl, heteroaralkyl, heteroalicyclyl, (heteroalicyclyl)alkyl, halogen, —C(=Z)R₁, —C(=Z)OR₁, —C(=Z)NR₁ₐR_{1b}, —C(=Z)N(R₁)NR₁ₐR_{1b}, —C(=Z)N(R₁)N(R₁)C(=Z)R₁, —C(R₁)═NR₁, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, and —OC(=Z)R₁, wherein any member of said group can be substituted or unsubstituted. In another embodiment, H can be selected from the group consisting of cycloalkyl, cycloalkenyl, aryl, heteroaryl, aralkyl, heteroaralkyl, heteroalicyclyl, (heteroalicyclyl)alkyl, hydroxyl, sulfenyl, sulfinyl, sulfonyl, haloalkoxy, —C(=Z)OR₁, —C(=Z)N(R₁)NR₁ₐR_{1b}, —C(=Z)N(R₁)N(R₁)C(=Z)R₁, —C(R₁)═NR₁, —NR₁ₐR_{1b}, —N═CR₁ₐR_{1b}, —S(O)NR₁ₐR_{1b}, —N(R₁)—S(═O)R₁, —N(R₁)—S(═O)₂R₁, and —OC(=Z)R₁, wherein any member of said group can be substituted or unsubstituted. In still another embodiment, H can be selected from the group consisting of cycloalkyl, aryl, heteroaryl, and heteroalicyclyl, wherein any member of said group can be substituted or unsubstituted. In yet still other embodiments, H can be an unsubstituted or substituted heteroaryl is selected from the group consisting of: 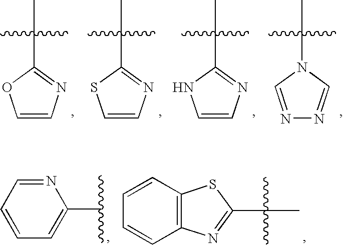
and 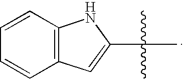
In one embodiment, H can be an optionally substituted phenyl. In certain embodiments, the optionally substituted phenyl can be substituted with a C₁₋₄ alkyl.

In some embodiments, H can be —C(=Z)NR₁ₐR_{1b}. In one embodiment, R₁ₐ can be selected from the group consisting of alkyl, aryl, aralkyl, heteroaryl, heteroaralkyl, heteroalicyclyl, (heteroalicyclyl)alkyl and —(CH₂)₀₋₇—NR₃ₐR_{3b}, wherein any member of said group can be substituted or unsubstituted. In certain embodiments, R₁ₐ can be selected from the group consisting of alkyl, alkoxy, aryl, aralkyl, heteroaryl, and heteroaralkyl, wherein any member of said group can be substituted or unsubstituted. In certain other embodiments, R₁ₐ can an optionally substituted heteroaryl or heteroaralkyl. In some of the embodiments, wherein H can be —C(=Z)NR₁ₐR_{1b} and alkyl, aryl, aralkyl, heteroaryl, heteroaralkyl, heteroalicyclyl, (heteroalicyclyl)alkyl and —(CH₂)₀₋₇—NR₃ₐR_{3b} then R_{1b} can be hydrogen or methyl. In particular embodiments, the optionally substituted heteroaryl or heteroaralkyl can be selected from the group consisting of: 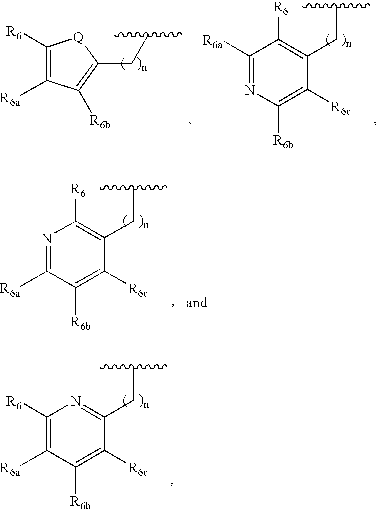
wherein Q is oxygen or sulfur, and in some embodiments, n can be 1 or 2. In more particular embodiments, the optionally substituted heteroaralkyl can be 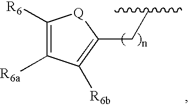
and in some embodiments, n can be 1 or 2.

In other embodiments, H can be —C(=Z)R₁ or —C(=Z)OR₁. In one embodiment, H can be —C(=Z)R₁ and R₁ can be selected from the group consisting of alkyl, cycloalkyl, aralkyl, halogen. In certain embodiments, H can be —C(=Z)OR₁ and R₁ can be alkyl or aralkyl.

In still other embodiments, H can —C(=Z)N(R₁)N(R₁)C(=Z)R₁ or —N(R₁)—C(=Z)NR₁ₐR_{1b}. In certain embodiments, —C(=Z)N(R₁)N(R₁)C(=Z)R₁ can be 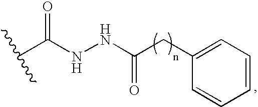
wherein n is 0 or 1. In certain other embodiments, H can be —N(R₁)—C(=Z)NR₁ₐR_{1b} and R₁ is hydrogen and R₁ₐ is alkyl or aralkyl. In any of the embodiments discussed in the present paragraph, R_{1b} can be hydrogen.

In yet still other embodiments, H can be selected from the group consisting of —C(R₁)═NR₁, —N(R₁)—C(=Z)R₁, and —OC(=Z)R₁. In certain embodiments, H can be —C(R₁)═NR₁, —N(R₁)—C(=Z)R₁, and —OC(=Z)R₁ wherein at least on R₁ is hydrogen or alkyl and at least one R₁ is selected from the group consisting of alkyl, aryl, and aralkyl.

In some embodiments, H can be —N(R₁)—S(═O)R₁ or —N(R₁)—S(═O)₂R₁. In certain embodiments, H can be —N(R₁)—S(═O)R₁ or —N(R₁)—S(═O)₂R₁ and R₁ can be hydrogen, aralkyl, or heteroaryl.

In other embodiments, H can be —S(O)NR₁ₐR_{1b} or —S(O)₂NR₁ₐR_{1b}. In certain embodiments, H can be —S(O)NR₁ₐR_{1b} or —S(O)₂NR₁ₐR_{1b} and R₁ₐ can be selected from the group consisting of alkyl, aryl, aralkyl, heteroaryl, and heteroalicyclyl. In any of the embodiments discussed in the present paragraph, R_{1b} can be hydrogen.

In one embodiments, H can be —S(O)NR₁ₐR_{1b}, —S(O)₂NR₁ₐR_{1b}, —C(=Z)NR₁ₐR_{1b} or —C(=Z)N(R₁)NR₁ₐR_{1b} and R₁, R₁ₐ and R_{1b} can each independently selected from the group consisting of:

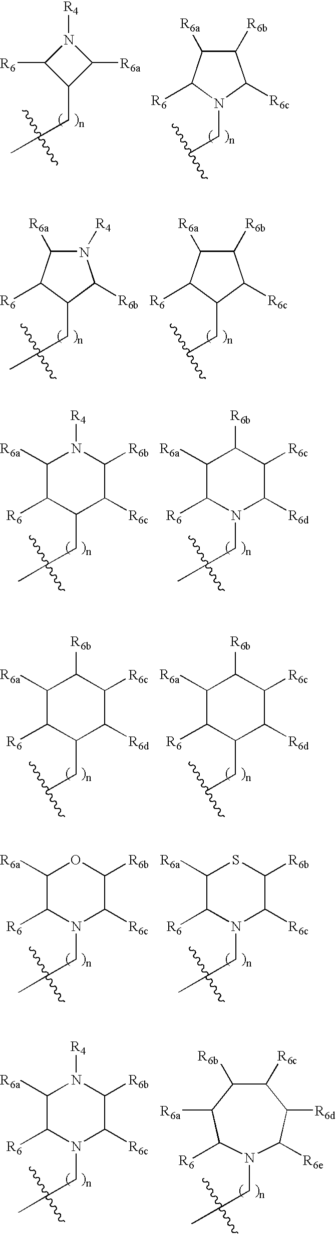

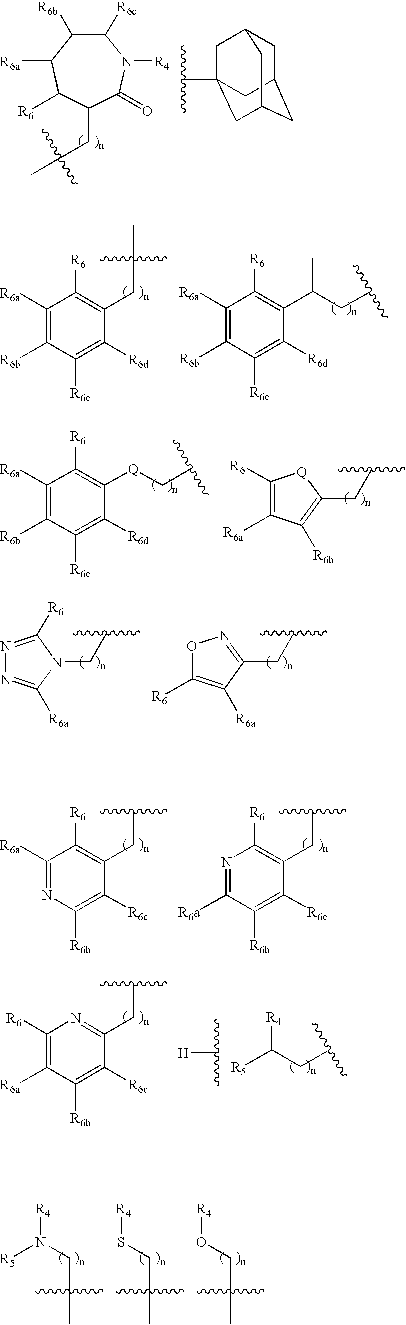

wherein:

n can be an integer selected from the group consisting of 0, 1, 2, 3, 4, 5, 6 or 7 defining the number of optionally substituted carbon atoms;

Q can be selected from the group consisting of —N(R₄)—, O and S;

R₄ and R₅ each each independently selected from the group consisting of hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino and protected amino; and

R₆, R₆ₐ, R_{6b}, R_{6c}, and R_{6d} can each independently selected from the group consisting of hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino and protected amino; or wherein the substituents selected from the group consisting of R₆, R₆ₐ, R_{6b}, R_{6c}, and R_{6d} can be taken together to form a cycloalkyl, cycloalkenyl, cycloalkynyl, or heteroalicyclyl ring with one or more adjacent members of said group consisting of R₆, R₆ₐ, R_{6b}, R_{6c}, and R_{6d}. In certain embodiments discussed in this paragarph, H can be —C(=Z)NR₁ₐR_{1b}. In certain embodiments discussed in this paragarph, H can be —C(=Z)NR₁ₐR_{1b} and n can be 0, 1, or 2. In any of the embodiments discussed in the present paragraph, R_{1b} can be hydrogen. In certain embodiments discussed in this paragarph, H can be —C(=Z)NR₁ₐR_{1b} and R_{1b} can be hydrogen. In certain embodiments discussed in this paragarph, H can be —C(=Z)NR₁ₐR_{1b}, R_{1b} can be hydrogen, and n can be 0, 1, or 2.

In some embodiments, R₁, R₁ₐ, R₂ₐ, R₂, R₃, R₃ₐ, and R_{3b} can be each independently selected from the group consisting of aryl, heteroaryl, heteroalicyclyl, aralkyl, heteraralkyl, or (heteroalicyclyl)alkyl and are substituted with zero to five substituents, wherein each substituent is independently selected from the group consisting of hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino and protected amino.

In one embodiment, A can be an aryl, heteroaryl, or heteroalicyclyl, and is substituted with zero to five substituents, wherein each substituent is independently selected from the group consisting of hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino, and protected amino. In certain embodiments, A can be an aryl, heteroaryl, or heteroalicyclyl and is substituted with zero to five substituents, wherein each substituent can be independently selected from the group consisting of alkyl, alkoxy, ester, cyano, and halogen. In some embodiments, the heteroaryl can be substituted or unsubstituted thiophene or substituted or unsubstituted pyridine. In other embodiments, the aryl can be an unsubstituted or substituted phenyl (e.g., 2-, 3-, 4-, 2-,3-, 2-,4- substituted phenyl). In certain embodiments when A is substituted phenyl, the phenyl can be substituted with a halogen, methoxy, or cyano group.

In some embodiments, X can be selected from the group consisting of S, S═O, and SO₂; Y can be —N(R₂) [symbol] or —C(R₁R₂) [symbol] ; the symbol [symbol] represents a single or double bond, where when [symbol] is a double bond, R₂ is absent; A can be selected from the group consisting of C₃-C₁₂alkyl, C₄-C₁₂alkyl, alkenyl, alkynyl, cycloalkyl, cycloalkenyl, cycloalkynyl, aryl, heteroaryl, aralkyl, and heteroaralkyl, wherein any member of said group can be substituted or unsubstituted; B, C, D, E, F, G and I can be separately selected from the group consisting of hydrogen, alkyl, alkenyl, alkynyl, cycloalkyl, cycloalkenyl, cycloalkynyl, aryl, heteroaryl, aralkyl, heteroaralkyl, heteroalicyclyl, (heteroalicyclyl)alkyl, halogen, hydroxyl, nitro, sulfenyl, sulfinyl, sulfonyl, haloalkyl, haloalkoxy, —CN, —C(=Z)R₁, —C(=Z)OR₁, —C(=Z)NR₁ₐR_{1b}, —C(=Z)N(R₁)NR₁ₐR_{1b}, —C(=Z)N(R₁)N(R₁)C(=Z)R₁, —C(R₁)═NR₁, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁, wherein any member of said group can be substituted or unsubstituted except for hydrogen; H can be selected from the group consisting of —C(=Z)NR₁ₐR_{1b}, —C(=Z)N(R₁)NR₁ₐR_{1b}, —C(=Z)N(R₁)N(R₁)C(=Z)R₁, and —C(R₁)═NR₁, wherein any member of said group can be substituted or unsubstituted; Z can be O or S; R₁, R₁ₐ and R_{1b} can each independently selected from the group consisting of: hydrogen, halogen, alkyl, alkenyl, alkynyl, cycloalkyl, cycloalkenyl, cycloalkynyl, aryl, aralkyl, heteroaryl, heteroaralkyl, heteroalicyclyl, (heteroalicyclyl)alkyl, —(CH₂)₀₋₇—OR₃, —(CH₂)₀₋₇—SR₃, —(CH₂)₀₋₇—NR₃ₐR_{3b}, haloalkyl, —C(=Z)R₃, —C(=Z)OR₃, and —C(=Z)NR₃ₐR_{3b}, wherein any member of said group can be substituted or unsubstituted except for hydrogen; or R₁ₐ and R_{1b} can be taken together to form an unsubstituted or substituted heteroalicyclyl having 2 to 9 carbon atoms or an unsubstituted or substituted carbocyclyl having 3 to 9 carbon atoms; R₂ can be absent or is selected from the group consisting of: hydrogen, alkyl, alkenyl, alkynyl, cycloalkyl, cycloalkenyl, cycloalkynyl, aryl, heteroaryl, and heteroalicyclyl, wherein any member of said group can be substituted or unsubstituted except for hydrogen; and R₃, R₃ₐ, and R_{3b} can each independently selected from the group consisting of: hydrogen, alkyl, alkenyl, alkynyl, cycloalkyl, cycloalkenyl, aryl, heteroaryl, heteroalicyclyl, aralkyl, heteroaralkyl, and (heteroalicyclyl)alkyl, wherein any member of said group can be substituted or unsubstituted except for hydrogen. In one embodiment, Z can be O (oxygen). In another embodiments, A can be selected from the group consisting of C₃-C₁₂alkyl (e.g., n-propyl), C₄-C₁₂alkyl (e.g., n-butyl), cycloalkyl (e.g, cyclohexyl), aryl (e.g., substituted or unsubstituted phenyl), and heteroaryl (e.g., thiophene and pyridine), wherein any member of said group can be substituted or unsubstituted. In yet another embodiment, Z can be O (oxygen) and A can be selected from the group consisting of C₃-C₁₂alkyl (e.g., n-propyl), C₄-C₁₂alkyl (e.g., n-butyl), cycloalkyl (e.g, cyclohexyl), aryl (e.g., substituted or unsubstituted phenyl), and heteroaryl (e.g., thiophene and pyridine), wherein any member of said group can be substituted or unsubstituted.

In some embodiments, A can be selected from the group consisting of C₃-C₁₂alkyl (e.g., n-propyl), C₄-C₁₂alkyl (e.g., n-butyl), cycloalkyl(e.g, cyclohexyl), aryl(e.g., substituted or unsubstituted phenyl), heteroaryl(e.g., thiophene and pyridine), heteroalicyclyl (e.g., piperidine), halogen, —NR₁ₐR_{1b}, and —(CH₂)₀₋₄—C(=Z)-OR₁. In other embodiments, A can be selected from the group consisting of C₃-C₁₂alkyl (e.g., n-propyl), C₄-C₁₂alkyl (e.g., n-butyl), cycloalkyl(e.g, cyclohexyl), aryl(e.g., substituted or unsubstituted phenyl), heteroaryl(e.g., thiophene and pyridine), heteroalicyclyl (e.g., piperidine), halogen, —NR₁ₐR_{1b}, and —(CH₂)₀₋₄—C(=Z)-OR₁; and X can be S (sulfur). In certain embodiments, A can be —NR₁ₐR_{1b} wherein R₁ₐ is an aryl (e.g.,optionally substituted phenyl) and R_{1b} is hydrogen. In certain other embodiments, A can be —NR₁ₐR_{1b} wherein R₁ₐ is a phenyl group substituted with a halogen and R_{1b} is hydrogen. In certain embodiments, A can be C₃-C₁₂alkyl (e.g., n-propyl), C₄-C₁₂alkyl (e.g., n-butyl). In certain other embodiments, A can be cycloalkyl (e.g, cyclohexyl). In other certain embodiments, A can be aryl (e.g., substituted or unsubstituted phenyl). In certain embodiments, the aryl can be an unsubstituted or substituted phenyl (e.g., 2-, 3-, 4-, 2-,3-, 2-,4- substituted phenyl) In certain other embodiments, A can be heteroaryl (e.g., optionally thiophene or optionally substituted pyridine). In some embodiments, A is not C₃-, C₄-, C₅-, C₆-, C₇-, C₈-, C₉-, C₁₀-, C₁₁-, C₁₂ alkyl. In other embodiments, A is not C₆-, C₇-, C₈-, C₉-, C₁₀-, C₁₁-, C₁₂ alkyl. In still other embodiments, A is not cycloalkyl. In some embodiments, A is not aryl. In other embodiments, A is not heteroaryl. In still other embodiments, A is not heteroalicyclyl. In yet still embodiments, A is not halogen, —NR₁ₐR_{1b}. In some embodiments, A is not —(CH₂)₀₋₄—C(=Z)-OR₁.

In some embodiments, A can be selected from the group consisting of C₃-C₁₂alkyl, C₄-C₁₂alkyl, cycloalkyl, aryl, heteroaryl, heteroalicyclyl, halogen, —NR₁ₐR_{1b}, and —(CH₂)₀₋₄—C(=Z)-OR₁; X can be S (sulfur); and Y can be —N(R₂) [symbol] wherein the symbol [symbol] represents a double bond and R₂ does not exist. In some embodiments, A can be selected from the group consisting of C₃-C₁₂alkyl, C₄-C₁₂alkyl, cycloalkyl, aryl, heteroaryl, heteroalicyclyl, halogen, —NR₁ₐR_{1b}, and —(CH₂)₀₋₄—C(=Z)-OR₁; X can be S; Y can be —N(R₂) [symbol] wherein the symbol [symbol] represents a double bond and R₂ does not exist; and H can be —C(=Z)NR₁ₐR_{1b}. In certain embodiments, A can be selected from the group consisting of C₃-C₁₂alkyl, C₄-C₁₂alkyl, halogen, and —(CH₂)₀₋₄—C(=Z)-OR₁; X can be S; Y can be —N(R₂) [symbol] wherein the symbol [symbol] represents a double bond and R₂ does not exist; and H can be —C(=Z)NR₁ₐR_{1b}. In certain other embodiments, A can be an aryl or a heteroaryl group; X can be S; Y can be —N(R₂) [symbol] wherein the symbol [symbol] represents a double bond and R₂ does not exist; and H can be —C(=Z)NR₁ₐR_{1b}. In certain embodiments, A can be a cycloalkyl, a heteroalicyclyl, or —NR₁ₐR_{1b} group; X can be S; Y can be —N(R₂) [symbol] wherein the symbol [symbol] represents a double bond and R₂ does not exist; and H can be —C(=Z)NR₁ₐR_{1b}. In some embodiments X can be S; Y can be —N(R₂) [symbol] wherein the symbol [symbol] represents a double bond and R₂ does not exist; and H can be —C(=Z)NR₁ₐR_{1b}, wherein R₁ₐ can be selected from the group consisiting of alkyl, alkoxy, cycloalkyl, cycloalkenyl, cycloalkynyl, aryl, aralkyl, heteroaryl, heteroaralkyl, heteroalicyclyl, (heteroalicyclyl)alkyl and —(CH₂)₀₋₇—NR₃ₐR_{3b}, wherein any member of said group can be substituted or unsubstituted.

In some embodiments, A can be selected from the group consisting of C₃-C₁₂alkyl, C₄-C₁₂alkyl, cycloalkyl, aryl, heteroaryl, heteroalicyclyl, halogen, —NR₁ₐR_{1b}, and —(CH₂)₀₋₄—C(=Z)-OR₁; X can be S; Y can be —N(R₂) [symbol] wherein the symbol [symbol] represents a double bond and R₂ does not exist; and H can be —C(=Z)NR₁ₐR_{1b}, wherein R₁ₐ can be an optionally substituted alkyl, alkoxy, or —(CH₂)₀₋₇—NR₃ₐR_{3b}. In other embodiments, A can be selected from the group consisting of C₃-C₁₂alkyl, C₄-C₁₂alkyl, halogen, and —(CH₂)₀₋₄—C(=Z)-OR₁; X can be S; Y can be —N(R₂) [symbol] wherein the symbol [symbol] represents a double bond and R₂ does not exist; and H can be —C(=Z)NR₁ₐR_{1b}, wherein R₁ₐ can be an optionally substituted alkyl, alkoxy, or —(CH₂)₀₋₇—NR₃ₐR_{3b}. In still other embodiments, A can be selected from the group consisting of aryl (e.g., unsubstituted or substituted phenyl) or a heteroaryl (e.g., thiophene and pyridine); X can be S; Y can be —N(R₂) [symbol] wherein the symbol [symbol] represents a double bond and R₂ does not exist; and H can be —C(=Z)NR₁ₐR_{1b}, wherein R₁ₐ can be an optionally substituted alkyl, alkoxy, or —(CH₂)₀₋₇—NR₃ₐR_{3b}. In yet still other embodiments, A can be selected from the group consisting of cycloalkyl (e.g., cyclohexyl), a heteroalicyclyl (e.g., piperidine), or —NR₁ₐR_{1b} group; X can be S; Y can be —N(R₂) [symbol] wherein the symbol [symbol] represents a double bond and R₂ does not exist; and H can be —C(=Z)NR₁ₐR_{1b}, wherein R₁ₐ can be an optionally substituted alkyl, alkoxy, or —(CH₂)₀₋₇—NR₃ₐR_{3b}. In certain embodiments, the alkyl can be C₁₋₆ alkyl. In certain other embodiments, the alkoxy is a C₁₋₆ alkoxy.

In some embodiments, A can be selected from the group consisting of C₃-C₁₂alkyl, C₄-C₁₂alkyl, cycloalkyl, aryl, heteroaryl, heteroalicyclyl, halogen, —NR₁ₐR_{1b}, and —(CH₂)₀₋₄—C(=Z)-OR₁; X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted cycloalkyl, cycloalkenyl, or cycloalkynyl. In other embodiments, A can be selected from the group consisting of C₃-C₁₂alkyl, C₄-C₁₂alkyl, halogen, and —(CH₂)₀₋₄—C(=Z)-OR₁; X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted cycloalkyl, cycloalkenyl, or cycloalkynyl. In still other embodiments, A can be selected from the group consisting of aryl (e.g., unsubstituted or substituted phenyl) or a heteroaryl (e.g., thiophene and pyridine); X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted cycloalkyl, cycloalkenyl, or cycloalkynyl. In yet still other embodiments, A can be selected from the group consisting of cycloalkyl (e.g., cyclohexyl), a heteroalicyclyl (e.g., piperidine), or —NR₁ₐR_{1b} group; X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted cycloalkyl, cycloalkenyl, or cycloalkynyl. In certain embodiments, the optionally substituted cycloalkyl, cycloalkenyl, or cycloalkynyl is selected from the group consisting of: 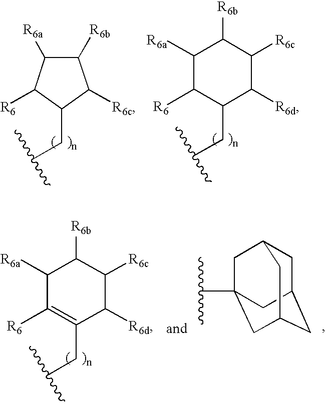
and in some of the embodiments, n can be 1 or 2.

In some embodiments, A can be selected from the group consisting of C₃-C₁₂alkyl, C₄-C₁₂alkyl, cycloalkyl, aryl, heteroaryl, heteroalicyclyl, halogen, —NR₁ₐR_{1b}, and —(CH₂)₀₋₄—C(=Z)-OR₁; X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted aryl or aralkyl. In other embodiments, A can be selected from the group consisting of C₃-C₁₂alkyl, C₄-C₁₂alkyl, halogen, and —(CH₂)₀₋₄—C(=Z)-OR₁; X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted aryl or aralkyl. In still other embodiments, A can be selected from the group consisting of aryl (e.g., unsubstituted or substituted phenyl) or a heteroaryl (e.g., thiophene and pyridine); X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted aryl or aralkyl. In yet still other embodiments, A can be selected from the group consisting of cycloalkyl (e.g., cyclohexyl), a heteroalicyclyl (e.g., piperidine), or —NR₁ₐR_{1b} group; X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted aryl or aralkyl. In certain embodiments, the optionally substituted aryl or aralkyl can be selected from the group consisting of: 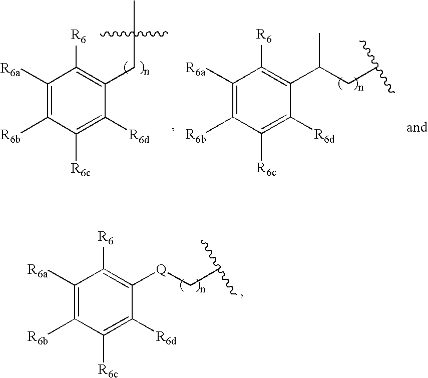
wherein Q can be —N(R₄)—, oxygen or sulfur; and R₄ can be hydrogen or C₁₋₄alkyl, and in some of the embodiments, n can be 1 or 2.

In some embodiments, A can be selected from the group consisting of C₃-C₁₂alkyl, C₄-C₁₂alkyl, cycloalkyl, aryl, heteroaryl, heteroalicyclyl, halogen, —NR₁ₐR_{1b}, and —(CH₂)₀₋₄—C(=Z)-OR₁; X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted heteroalicyclyl or (heteroalicyclyl)alkyl. In other embodiments, A can be selected from the group consisting of C₃-C₁₂alkyl, C₄-C₁₂alkyl, halogen, and —(CH₂)₀₋₄—C(=Z)-OR₁; X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted heteroalicyclyl or (heteroalicyclyl)alkyl. In still other embodiments, A can be selected from the group consisting of aryl (e.g., unsubstituted or substituted phenyl) or a heteroaryl (e.g., thiophene and pyridine); X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted heteroalicyclyl or (heteroalicyclyl)alkyl. In yet still other embodiments, A can be selected from the group consisting of cycloalkyl (e.g., cyclohexyl), a heteroalicyclyl (e.g., piperidine), or —NR₁ₐR_{1b} group; X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted heteroalicyclyl or (heteroalicyclyl)alkyl. In certain embodiments, the optionally substituted heteroalicyclyl or (heteroalicyclyl)alkyl can be selected from the group consisting of: 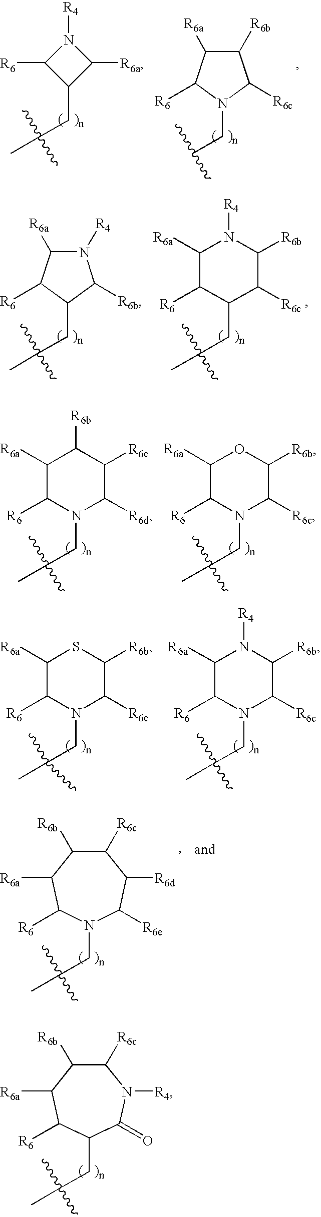
and in some of the embodiments, n can be 1 or 2.

In some embodiments, A can be selected from the group consisting of C₃-C₁₂alkyl, C₄-C₁₂alkyl, cycloalkyl, aryl, heteroaryl, heteroalicyclyl, halogen, —NR₁ₐR_{1b}, and —(CH₂)₀₋₄—C(=Z)-OR₁; X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted heteroaryl or heteroaralkyl. In other embodiments, A can be selected from the group consisting of C₃-C₁₂alkyl, C₄-C₁₂alkyl, halogen, and —(CH₂)₀₋₄—C(=Z)-OR₁; X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted heteroaryl or heteroaralkyl. In still other embodiments, A can be selected from the group consisting of aryl (e.g., unsubstituted or substituted phenyl) or a heteroaryl (e.g., thiophene and pyridine); X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted heteroaryl or heteroaralkyl. In yet still other embodiments, A can be selected from the group consisting of cycloalkyl (e.g., cyclohexyl), a heteroalicyclyl (e.g., piperidine), or —NR₁ₐR_{1b} group; X can be S; Y can be —N(R₂) [symbol] wherein the symbol [symbol] represents a double bond and R₂ does not exist; and H can be —C(=Z)NR₁ₐR_{1b}, wherein R₁ₐ is an optionally substituted heteroaryl or heteroaralkyl. In certain embodiments, the optionally substituted heteroaralkyl is from the group consisting of: 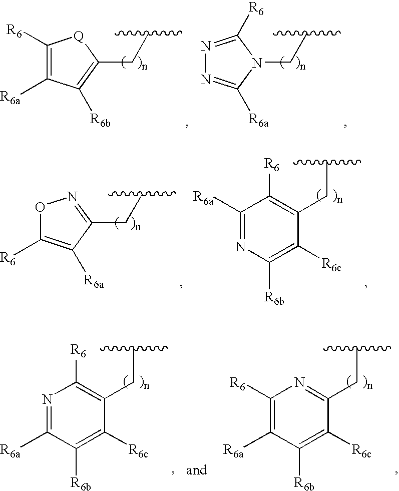
wherein Q can be oxygen or sulfur, and in some of the embodiments, n can be 1 or 2. In certain other embodiments, the optionally substituted heteroaralkyl can be 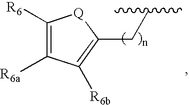
wherein Q can be oxygen or sulfur, and in some of the embodiments, n can be 1 or 2.

Another embodiment described herein relates to each of the compounds and formulae shown in the claims. In one embodiment, the compound of Formula (I) can be selected from the group consisting of:

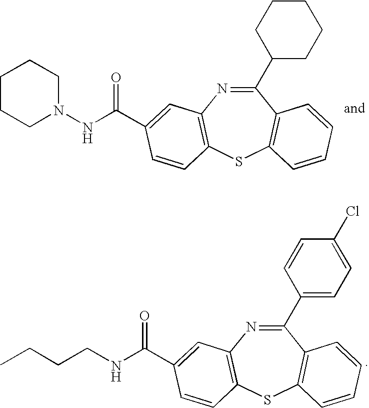

Certain of the compounds of the present invention may exist as stereoisomers including optical isomers. The invention includes all stereoisomers and both the racemic mixtures of such stereoisomers as well as the individual enantiomers that may be separated according to methods that are well known to those of ordinary skill in the art.

In some embodiments, the compound of Formula (I) can bind to a cannabinoid receptor. Preferably, in some embodiments, the cannabinoid receptor can be a CB1 receptor.

Still another embodiment described herein relates to a pharmaceutical composition, comprising a therapeutically effective amount of a compound of Formula (I) and a pharmaceutically acceptable carrier, diluent, or excipient.

### DEFINITIONS

Unless defined otherwise, all technical and scientific terms used herein have the same meaning as is commonly understood by one of ordinary skill in the art to which this invention belongs. All patents, applications, published applications and other publications referenced herein are incorporated by reference in their entirety. In the event that there are plurality of definitions for a term herein, those in this section prevail unless stated otherwise

As used herein, any “R” group(s) such as, without limitation, R₁, R₁ₐ and R_{1b}, represent substituents that can be attached to the indicated atom. A non-limiting list of R groups include but are not limited to hydrogen, alkyl, alkenyl, alkynyl, cycloalkyl, cycloalkenyl, cycloalkynyl, aryl, heteroaryl, and heteroalicyclyl. An R group may be substituted or unsubstituted. If two “R” groups are covalently bonded to the same atom or to adjacent atoms, then they may be “taken together” as defined herein to form a cycloalkyl, aryl, heteroaryl or heteroalicyclyl group. For example, without limitation, if Rₐ and R_{b} of an NRₐR_{b} group are indicated to be “taken together”, it means that they are covalently bonded to one another at their terminal atoms to form a ring that includes the nitrogen:

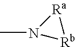

As used herein, “IC₅₀” refers to an amount, concentration, or dosage of a particular test compound that achieves a 50% inhibition of a maximal response, such as modulation of GPCR, including cannabinoid receptor, activity an assay that measures such response. The assay may be an R-SAT® assay as described herein but is not limited to an RSAT assay.

As used herein, “EC₅₀” refers to an amount, concentration, or dosage of a particular test compound that elicits a dose-dependent response at 50% of maximal expression of a particular response that is induced, provoked or potentiated by the particular test compound, in an assay that measures such response such as but not limited to R-SAT® assay described herein.

Whenever a group of this invention is described as being “optionally substituted” that group may be unsubstituted or substituted with one or more of the indicated substituents. Likewise, when a group is described as being “unsubstituted or substituted” if substituted, the substituent may be selected from one or mmore of the indicated substituents.

Unless otherwise indicated, when a substituent is deemed to be “optionally subsituted,” or “substituted” it is meant that the subsitutent is a group that may be substituted with one or more group(s) individually and independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. The protecting groups that may form the protective derivatives of the above substituents are known to those of skill in the art and may be found in references Greene and Wuts, Protective Groups in Organic Synthesis, 3^{rd} Ed., John Wiley & Sons, New York, N.Y., 1999, which is hereby incorporated by reference in its entirety.

As used herein, “Cₘ to Cₙ ” in which “m” and “n” are integers refers to the number of carbon atoms in an alkyl, alkenyl or alkynyl group or the number of carbon atoms in the ring of a cycloalkyl or cycloalkenyl group. That is, the alkyl, alkenyl, alkynyl, ring of the cycloalkyl or ring of the cycloalkenyl can contain from “m” to “n”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “m” and “n” are designated with regard to an alkyl, alkenyl, alkynyl, cycloalkyl or cycloalkenyl group, the broadest range described in these definitions is to be assumed.

As used herein, “alkyl” refers to a straight or branched hydrocarbon chain fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 5 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, ethenyl, propenyl, butenyl, and the like.

The alkyl group may be substituted or unsubstituted. When substituted, the substituent group(s) is(are) one or more group(s) individually and independently selected from alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof. Wherever a substituent is described as being “optionally substituted” that substitutent may be substituted with one of the above substituents.

As used herein, “alkenyl” refers to an alkyl group that contains in the straight or branched hydrocarbon chain one or more double bonds. An alkenyl group of this invention may be unsubstituted or substituted. When substituted, the substituent(s) may be selected from the same groups disclosed above with regard to alkyl group substitution.

As used herein, “alkynyl” refers to an alkyl group that contains in the straight or branched hydrocarbon chain one or more triple bonds. An alkynyl group of this invention may be unsubstituted or substituted. When substituted, the substituent(s) may be selected from the same groups disclosed above with regard to alkyl group substitution

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, naphthalene and azulene. An aryl group of this invention may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

As used herein, “heteroaryl” refers to a monocyclic or multicyclic aromatic ring system (a ring system with fully delocalized pi-electron system), one or two or more fused rings that contain(s) one or more heteroatoms, that is, an element other than carbon, including but not limited to, nitrogen, oxygen and sulfur. Examples of heteroaryl rings include, but are not limited to, furan, thiophene, phthalazine, pyrrole, oxazole, thiazole, imidazole, pyrazole, isoxazole, isothiazole, triazole, thiadiazole, pyridine, pyridazine, pyrimidine, pyrazine and triazine. A heteroaryl group of this invention may be substituted or unsubstituted. When substituted, hydrogen atoms are replaced by substituent group(s) that is(are) one or more group(s) independently selected from alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, and amino, including mono- and di-substituted amino groups, and the protected derivatives thereof.

An “aralkyl” is an aryl group connected, as a substituent, via a lower alkylene group. The lower alkylene and aryl group of an aralkyl may be substituted or unsubstituted. Examples include but are not limited to benzyl, substituted benzyl, 2-phenylethyl, 3-phenylpropyl, and naphtylalkyl.

A “heteroaralkyl” is heteroaryl group connected, as a substituent, via a lower alkylene group. The lower alkylene and heteroaryl group of heteroaralkyl may be substituted or unsubstituted. Examples include but are not limited to 2-thienylmethyl, 3-thienylmethyl, furylmethyl, thienylethyl, pyrrolylalkyl, pyridylalkyl, isoxazollylalkyl, and imidazolylalkyl, and their substituted as well as benzo-fused analogs.

“Lower alkylene groups” are straight-chained tethering groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—(CH₂)₄—) groups. A lower alkylene group may be substituted or unsubstituted.

As used herein, “alkylidene” refers to a divalent group, such as ═CR′R″, which is attached to one carbon of another group, forming a double bond, Alkylidene groups include, but are not limited to, methylidene (═CH₂) and ethylidene (═CHCH₃). As used herein, “arylalkylidene” refers to an alkylidene group in which either R′ and R″ is an aryl group. An alkylidene group may be substituted or unsubstituted.

As used herein, “alkoxy” refers to the formula —OR wherein R is an alkyl is defined as above, e.g. methoxy, ethoxy, n-propoxy, 1-methylethoxy(isopropoxy), n-butoxy, iso-butoxy, sec-butoxy, tert-butoxy, amoxy, tert-amoxy and the like. An alkoxy may be substituted or unsubstituted.

As used herein, “alkylthio” refers to the formula —SR wherein R is an alkyl is defined as above, e.g. methylmercapto, ethylmercapto, n-propylmercapto, 1-methylethylmercapto (isopropylmercapto), n-butylmercapto, iso-butylmercapto, sec-butylmercapto, tert-butylmercapto, and the like. An alkylthio may be substituted or unsubstituted.

As used herein, “aryloxy” and “arylthio” refers to RO—and RS—, in which R is an aryl, such as but not limited to phenyl. Both an aryloxyl and arylthio may be substituted or unsubstituted.

As used herein, “acyl” refers to a hydrogen, alkyl, alkenyl, alkynyl, or aryl connected, as substituents, via a carbonyl group. Examples include formyl, acetyl, propanoyl, benzoyl, and acryl. An acyl may be substituted or unsubstituted. An acyl may be substituted or unsubstituted.

As used herein, “cycloalkyl” refers to a completely saturated (no double bonds) mono- or multi-cyclic hydrocarbon ring system. When composed of two or more rings, the rings may be joined together in a fused, bridged or spiro-connected fashion. Cycloalkyl groups of this invention may range from C₃ to C₁₀, in other embodiments it may range from C₃ to C₆. A cycloalkyl group may be unsubstituted or substituted. Typical cycloalkyl groups include, but are in no way limited to, cyclopropyl, cyclobutyl, cyclopentyl, cyclohexyl, and the like. If substituted, the substituent(s) may be an alkyl or selected from those indicated above with regard to substitution of an alkyl group unless otherwise indicated.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). When composed of two or more rings, the rings may be connetected together in a fused, bridged or spiro-connected fashion. A cycloalkenyl group of this invention may be unsubstituted or substituted. When substituted, the substituent(s) may be an alkyl or selected from the groups disclosed above with regard to alkyl group substitution unless otherwise indicated.

As used herein, “cycloalkynyl” refers to a cycloalkyl group that contains one or more triple bonds in the ring. When composed of two or more rings, the rings may be joined together in a fused, bridged or spiro-connected fashion. A cycloalkynyl group of this invention may be unsubstituted or substituted. When substituted, the substituent(s) may be an alkyl or selected from the groups disclosed above with regard to alkyl group substitution unless otherwise indicated.

As used herein, “heteroalicyclic” or “heteroalicyclyl” refers to a stable 3- to 18 membered ring which consists of carbon atoms and from one to five heteroatoms selected from the group consisting of nitrogen, oxygen and sulfur. For the purpose of this invention, the “heteroalicyclic” or “heteroalicyclyl” may be monocyclic, bicyclic, tricyclic, or tetracyclic ring system, which may be joined together in a fused, bridged or spiro-connected fashion; and the nitrogen, carbon and sulfur atoms in the “heteroalicyclic” or “heteroalicyclyl” may be optionally oxidized; the nitrogen may be optionally quaternized; and the rings may also contain one or more double bonds provided that they do not form a fully delocalized pi-electron system throughout all the rings. Heteroalicyclyl groups of this invention may be unsubstituted or substituted. When substituted, the substituent(s) may be one or more groups independently selected from the group consisting of alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, haloalkyl, haloalkoxy, trihalomethanesulfonyl, trihalomethanesulfonamido, and amino, including mono- and di-substituted amino groups, and the protected derivatives thereof. Examples of such “heteroalicyclic” or “heteroalicyclyl” include but are not limited to, azepinyl, acridinyl, carbazolyl, cinnolinyl, dioxolanyl, imidazolinyl, morpholinyl, oxiranyl, piperidinyl N-Oxide, piperidinyl, piperazinyl, pyrrolidinyl, 4-piperidonyl, pyrazolidinyl, 2-oxopyrrolidinyl, thiamorpholinyl, thiamorpholinyl sulfoxide, and thiamorpholinyl sulfone.

A “(cycloalkyl)alkyl” is a cycloalkyl group connected, as a substituent, via a lower alkylene group. The lower alkylene and cycloalkyl of a (cycloalkyl)alkyl may be substituted or unsubstituted. Examples include but are not limited cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like.

A “(cycloalkenyl)alkyl” is a cycloalkenyl group connected, as a substituent, via a lower alkylene group. The lower alkylene and cycloalkenyl of a (cycloalkenyl)alkyl may be substituted or unsubstituted.

A “(cycloalkynyl)alkyl” is a cycloalkynyl group connected, as a substituent, via a lower alkylene group. The lower alkylene and cycloalkynyl of a (cycloalkynyl)alkyl may be substituted or unsubstituted.

As used herein, “halo” or “halogen” refers to F (fluoro), Cl (chloro), Br (bromo) or I (iodo).

As used herein, “haloalkyl” refers to an alkyl group in which one or more of the hydrogen atoms are replaced by halogen. Such groups include but are not limited to, chloromethyl, fluoromethyl, difluoromethyl, trifluoromethyl and 1-chloro-2-fluoromethyl, 2-fluoroisobutyl. A haloalkyl may be substituted or unsubstituted.

As used herein, “haloalkoxy” refers to RO-group in which R is a haloalkyl group. Such groups include but are not limited to, chloromethoxy, fluoromethoxy, difluoromethoxy, trifluoromethoxy and 1-chloro-2-fluoromethoxy, 2-fluoroisobutyoxy. A haloalkoxy may be substituted or unsubstituted.

An “O-carboxy” group refers to a “RC(═O)O—” group in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

A “C-carboxy” group refers to a “—C(═O)R” group in which R can be the same as defined with respect to O-carboxy. A C-carboxy may be substituted or unsubstituted.

A “trihalomethanesulfonyl” group refers to an “X₃CSO₂—” group wherein X is a halogen.

A “cyano” group refers to a “—CN” group.

An “isocyanato” group refers to a “—NCO” group.

A “thiocyanato” group refers to a “—CNS” group.

An “isothiocyanato” group refers to an “—NCS” group.

A “sulfinyl” group refers to an “—S(═O)—R” group in which R can be the same as defined with respect to O-carboxy. A sulfinyl may be substituted or unsubstituted.

A “sulfonyl” group refers to an “SO₂R” group in which R can be the same as defined with respect to O-carboxy. A sulfonyl may be substituted or unsubstituted.

An “S-sulfonamido” group refers to a “—SO₂NR_{A}R_{B}” group in which R_{A} and R_{B} can be the same as defined with respect to O-carboxy. An S-sulfonamido may be substituted or unsubstituted.

An “N-sulfonamido” group refers to a “RSO₂N(R_{A})—” group in which R and R_{A} can be the same as defined with respect to O-carboxy. A sulfonyl may be substituted or unsubstituted.

A “trihalomethanesulfonamido” group refers to an “X₃CSO₂N(R)—” group with X as halogen and R can be the same as defined with respect to O-carboxy. A trihalomethanesulfonamido may be substituted or unsubstituted.

An “O-carbamyl” group refers to a “—OC(═O)NR_{A}R_{B}” group in which R_{A} and R_{B} can be the same as defined with respect to O-carboxy. An O-carbamyl may be substituted or unsubstituted.

An “N-carbamyl” group refers to an “ROC(═O)NR_{A}—” group in which R and R_{A} can be the same as defined with respect to O-carboxy. An N-carbamyl may be substituted or unsubstituted.

An “O-thiocarbamyl” group refers to a “—OC(═S)—NR_{A}R_{B}” group in which R_{A} and R_{B} can be the same as defined with respect to O-carboxy. An O-thiocarbamyl may be substituted or unsubstituted.

An “N-thiocarbamyl” group refers to an “ROC(═S)NR_{A}—” group in which R and R_{A} can be the same as defined with respect to O-carboxy. An N-thiocarbamyl may be substituted or unsubstituted.

A “C-amido” group refers to a “—C(═O)NR_{A}R_{B}” group in which R_{A} and R_{B} can be the same as defined with respect to O-carboxy. A C-amido may be substituted or unsubstituted.

An “N-amido” group refers to a “RC(═O)NR_{A}—” group in which R and R_{A} can be the same as defined with respect to O-carboxy. An N-amido may be substituted or unsubstituted.

An “ester” refers to a “—C(═O)OR” group in which R can be the same as defined with respect to O-carboxy. An ester may be substituted or unsubstituted.

A lower aminoalkyl refers to an amino group connected via a lower alkylene group. A lower aminoalkyl may be substituted or unsubstituted.

A lower alkoxyalkyl refers to an alkoxy group connected via a lower alkylene group. A lower alkoxyalkyl may be substituted or unsubstituted.

Any unsubstituted or monosubstituted amine group on a compound herein can be converted to an amide, any hydroxyl group can be converted to an ester and any carboxyl group can be converted to either an amide or ester using techniques well-known to those skilled in the art (see, for example, Greene and Wuts, Protective Groups in Organic Synthesis, 3^{rd} Ed., John Wiley & Sons, New York, N.Y., 1999).

Where the numbers of substituents are not specified (e.g. haloalkyl), there may be one or more substituents present. For example “haloalkyl” may include one or more of the same or different halogens. As another example, “C₁-C₃ alkoxyphenyl” may include one or more of the same or different alkoxygroups containing one, two or three atoms.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

As employed herein, the following terms have their accepted meaning in the chemical literature.

| | |
|---|---|
| AcOH | acetic acid |
| anhyd | anhydrous |
| CDI | 1,1′-carbonyldiimidazole |
| DCM | dichloromethane |
| DMF | N,N-dimethylformamide |
| DMSO | dimethyl sulfoxide |
| Et₂O | diethyl ether |
| EtOAc | ethyl acetate |
| EtOH | Ethanol |
| MeOH | Methanol |
| NH₄OAc | ammonium acetate |
| Pd/C | palladium on activated carbon |

It is understood that, in any compound of this invention having one or more chiral centers, if an absolute stereochemistry is not expressly indicated, then each center may independently be of R-configuration or S-configuration or a mixture thereof. Thus, the compounds provided herein may be enatiomerically pure or be stereoisomeric mixtures. In addition it is understood that, in any compound of this invention having one or more double bond(s) generating geometrical isomers that can be defined as E or Z each double bond may independently be E or Z a mixture thereof. Likewise, all tautomeric forms are also intended to be included.

As used herein, “pharmaceutically acceptable salt” refers to a salt of a compound that does not abrogate the biological activity and properties of the compound. Pharmaceutical salts can be obtained by reaction of a compound disclosed herein with an acid or base. Base-formed salts include, without limitation, ammonium salt (NH₄⁺); alkali metal, such as, without limitation, sodium or potassium, salts; alkaline earth, such as, without limitation, calcium or magnesium, salts; salts of organic bases such as, without limitation, dicyclohexylamine, N-methyl-D-glucamine, tris(hydroxymethyl)methylamine; and salts with the amino group of amino acids such as, without limitation, arginine and lysine. Useful acid-based salts include, without limitation, hydrochlorides, hydrobromides, sulfates, nitrates, phosphates, methanesulfonates, ethanesulfonates, p-toluenesulfonates and salicylates.

Pharmaceutically acceptable solvates and hydrates are complexes of a compound with one or more solvent of water molecules, or 1 to about 100, or 1 to about 10, or one to about 2, 3 or 4, solvent or water molecules.

As used herein, a “prodrug” refers to a compound that may not be pharmaceutically active but that is converted into an active drug upon in vivo administration. The prodrug may be designed to alter the metabolic stability or the transport characteristics of a drug, to mask side effects or toxicity, to improve the flavor of a drug or to alter other characteristics or properties of a drug. Prodrugs are often useful because they may be easier to administer than the parent drug. They may, for example, be bioavailable by oral administration whereas the parent drug is not. The prodrug may also have better solubility than the active parent drug in pharmaceutical compositions. An example, without limitation, of a prodrug would be a compound disclosed herein, which is administered as an ester (the “prodrug”) to facilitate absorption through a cell membrane where water solubility is detrimental to mobility but which then is metabolically hydrolyzed to a carboxylic acid (the active entity) once inside the cell where water-solubility is beneficial. A further example of a prodrug might be a short peptide (polyaminoacid) bonded to an acid group where the peptide is metabolized in vivo to release the active parent compound. By virtue of knowledge of pharmacodynamic processes and drug metabolism in vivo, those skilled in the art, once a pharmaceutically active compound is known, can design prodrugs of the compound (see, e.g. Nogrady (1985) Medicinal Chemistry A Biochemical Approach, Oxford University Press, New York, pages 388-392)

As used herein, the term “complement” refers to a oligonucleotide or polynucleotide that hybridizes by base-pairing, adenine to tyrosine and guanine to cytosine, to another oligonucleotide.

As used herein, to “modulate” the activity of CB1 means either to activate it, i.e., to increase its cellular function over the base level measured in the particular environment in which it is found, or deactivate it, i.e., decrease its cellular function to less than the measured base level in the environment in which it is found and/or render it unable to perform its cellular function at all, even in the presence of a natural binding partner. A natural binding partner is an endogenous molecule that is an agonist for the receptor.

As used herein, to “detect” changes in the activity of CB1 or of a CB1 sub-type refers to the process of analyzing the result of an experiment using whatever analytical techniques are best suited to the particular situation. In some cases simple visual observation may suffice, in other cases the use of a microscope, visual or UV light analyzer or specific protein assays may be required. The proper selection of analytical tools and techniques to detect changes in the activity of CB1 or a CB1 sub-type are well-known to those skilled in the art.

An “agonist” is defined as a compound that increases the basal activity of a receptor (i.e. signal transduction mediated by the receptor).

As used herein, “partial agonist” refers to a compound that has an affinity for a receptor but, unlike an agonist, when bound to the receptor it elicits only a fractional degree of the pharmacological response normally associated with the receptor even if a large number of receptors are occupied by the compound.

An “inverse agonist” is defined as a compound, which reduces, or suppresses the basal activity of a receptor, such that the compound is not technically an antagonist but, rather, is an agonist with negative intrinsic activity.

As used herein, “antagonist” refers to a compound that binds to a receptor to form a complex that does not give rise to any response, as if the receptor was unoccupied. An antagonist attenuates the action of an agonist on a receptor. An antagonist may bind reversibly or irreversibly, effectively eliminating the activity of the receptor permanently or at least until the antagonist is metabolized or dissociates or is otherwise removed by a physical or biological process.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice; rats; rabbits; guinea pigs; dogs; cats; sheep; goats; cows; horses; primates, such as monkeys, chimpanzees, and apes, and, in particular, humans.

As used herein, a “patient” refers to a subject that is being treated by a medical professional such as an M.D. or a D.V.M. to attempt to cure, or at least ameliorate the effects of, a particular disease or disorder or to prevent the disease or disorder from occurring in the first place.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

Synthesis

General synthetic routes to the compounds of this invention are shown in Schemes 1-7. The routes shown are illustrative only and are not intended, nor are they to be construed, to limit the scope of this invention in any manner whatsoever. Those skilled in the art will be able to recognize modifications of the disclosed synthesis and to devise alternate routes based on the disclosures herein; all such modifications and alternate routes are within the scope of this invention.

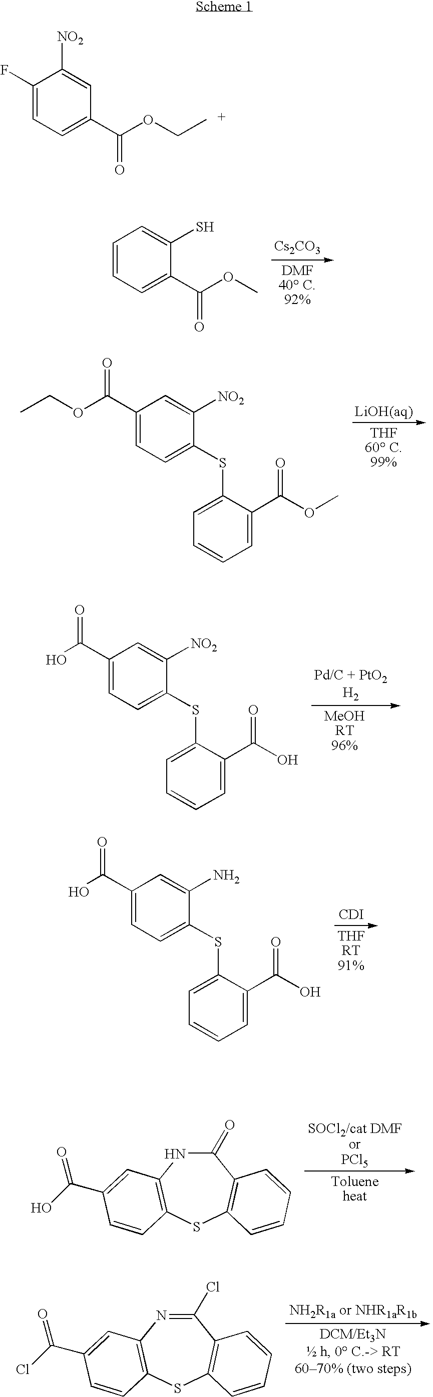

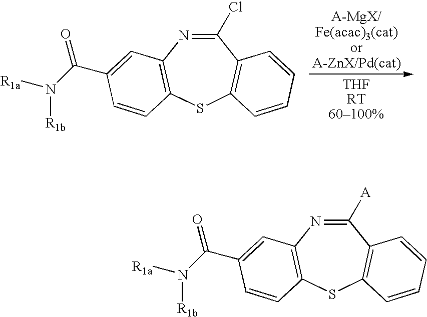

In Scheme 1, R₁ₐ, R_{1b}, and A are as defined above for Formula I.

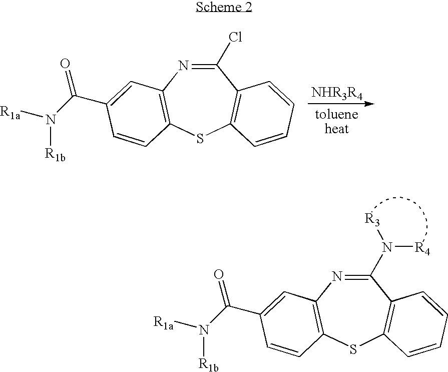

In Scheme 2, R₁ₐ and R_{1b} are as defined above for Formula I. R₃ and R₄ can be selected from the same group of substituents as R₁ₐ and R_{1b} as defined above for Formula I.

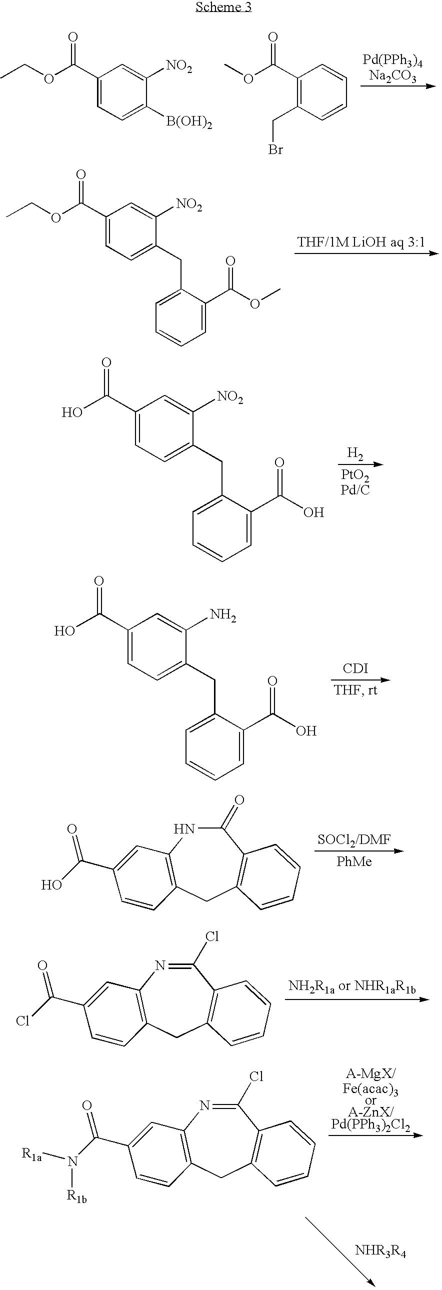

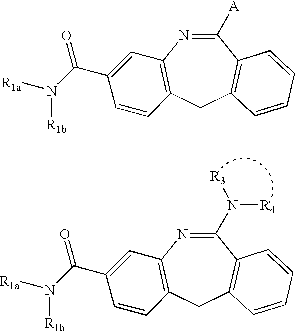

In Scheme 3, R₁ₐ, R_{1b}, and A are as defined above for Formula I. R₃ and R₄ can be selected from the same group of substituents as R₁ₐ and R_{1b} as defined above for Formula I.

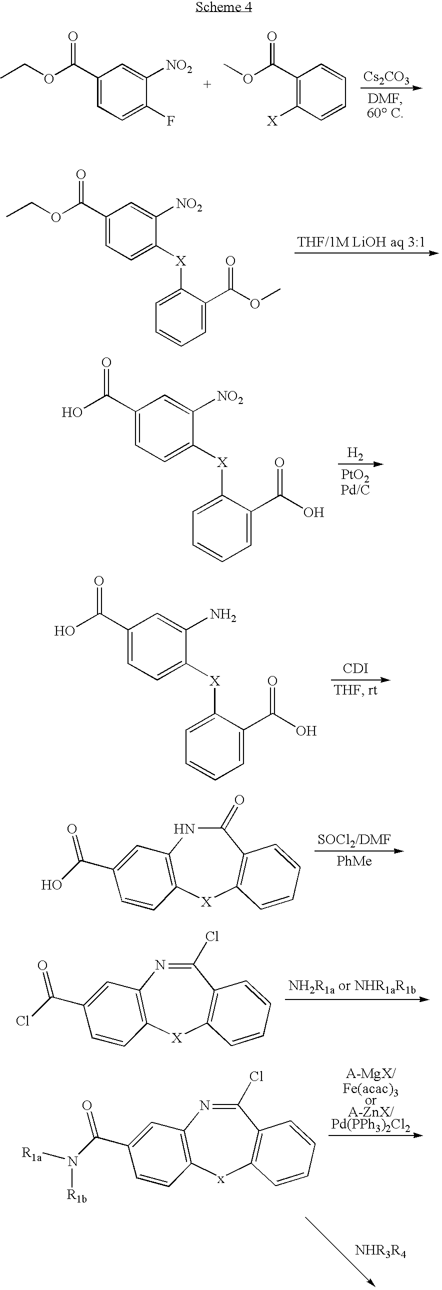

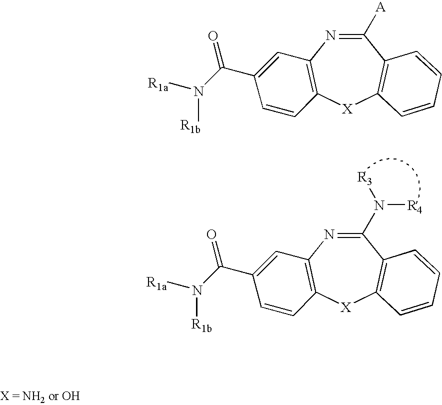

In Scheme 4, R₁ₐ, R_{1b}, and A are as defined above for Formula I. R₃ and R₄ can be selected from the same group of substituents as R₁ₐ and R_{1b} as defined above for Formula I.

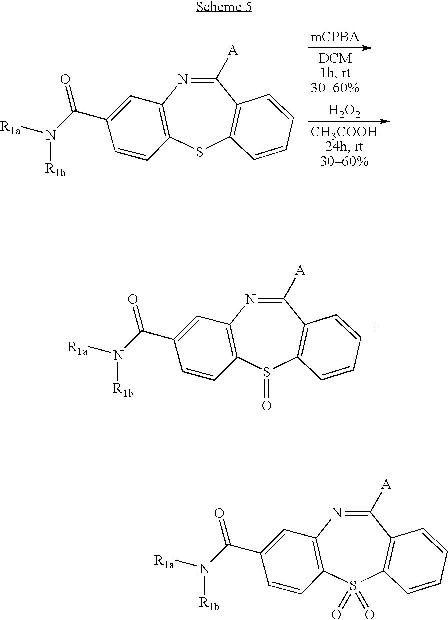

In Scheme 5, R₁ₐ, R_{1b}, and A are as defined above for Formula I.

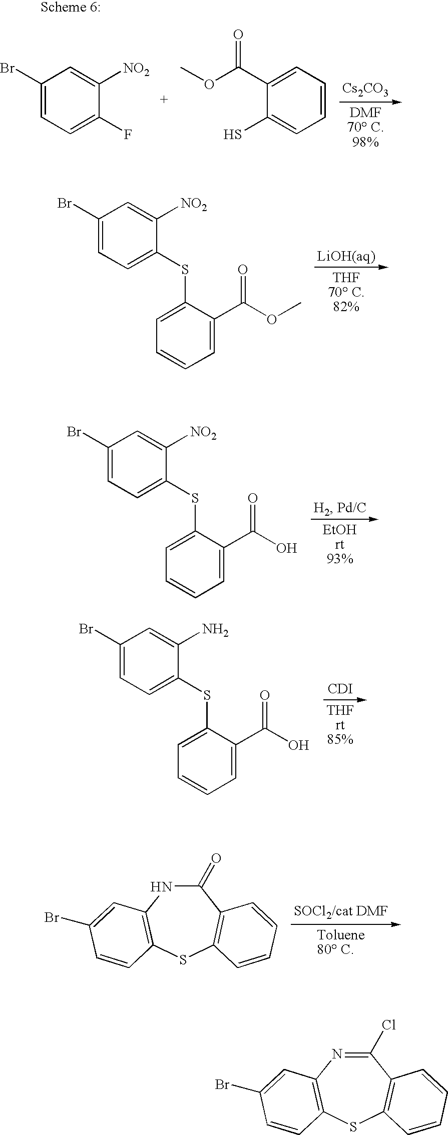

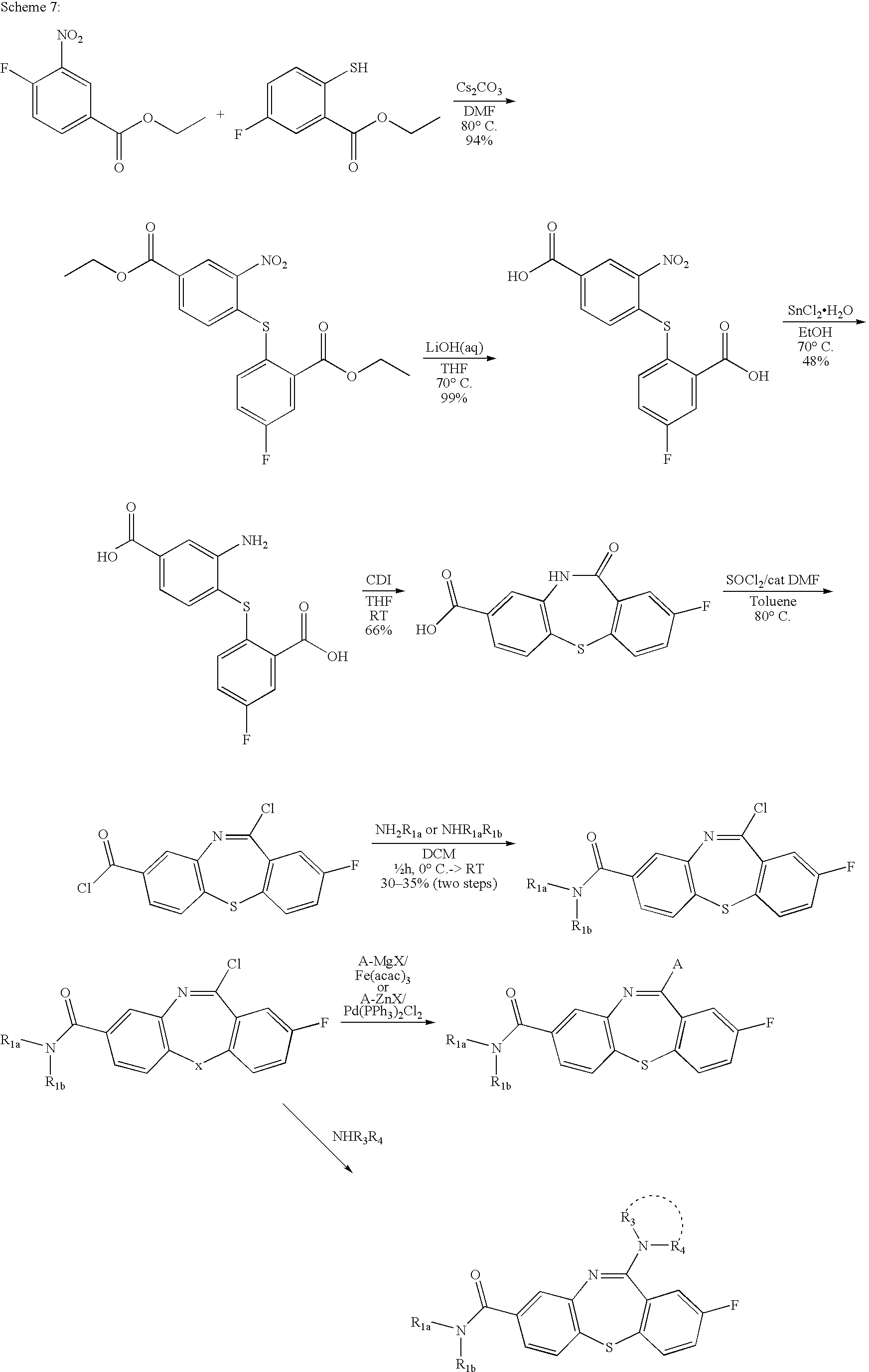

In Scheme 7, R₁ₐ, R_{1b}, and A are as defined above for Formula I. R₃ and R₄ can be selected from the same group of substituents as R₁ₐ and R_{1b} as defined above for Formula I.

Methods of Use

The term “therapeutically effective amount” is used to indicate an amount of an active compound, or pharmaceutical agent, that elicits the biological or medicinal response indicated. This response may occur in a tissue, system, animal or human that is being sought by a researcher, veterinarian, medical doctor or other clinician, and includes alleviation of the symptoms of the disease being treated.

One embodiment disclosed herein relates to a method of ameliorating or preventing a disease or condition by administering to a subject a therapeutically effective amount of one or more compounds of Formula I. The disease or condition can be selected from the group consisting of: a method of treating or preventing obesity, metabolic syndrome, a metabolic disorder, hypertension, polycystic ovary disease, osteoarthritis, a dermatological disorder, hypertension, insulin resistance, hypercholesterolemia, hypertriglyceridemia, cholelithiasis, a sleep disorder, hyperlipidemic conditions, bulimia nervosa, a compulsive eating disorder, an appetite disorder, atherosclerosis, diabetes, high cholesterol, hyperlipidemia, cachexia, an inflammatory disease, rheumatoid arthritis, a neurological disorder, a psychiatric disorder, substance abuse (e.g., alcohol, amphetamines, amphetamine-like substances, caffeine, cannabis, cocaine, hallucinogens, inhalents, nicotine, opioids, phencyclidine, phencyclidine-like compounds, sedative-hypnotics or benzodiazepines, and/or other unknown substances), depression, anxiety, mania, schizophrenia, dementia, dystonia, muscle spasticity, tremor, psychosis, an attention deficit disorder, a memory disorder, a cognitive disorder, short term memory loss, memory impairment (e.g., associated with dementia, Alzheimer's disease, schizophrenia, Parkinson's disease, Huntington's disease, Pick's disease, Creutzfeld-Jakob disease, HIV, cardiovascular disease, head trauma and/or age-related cognitive decline), drug addiction, alcohol addiction, nicotine addiction, infertility, hemorrhagic shock, septic shock, cirrhosis, a cardiovascular disorder, cardiac dysfunction, valvular disease, myocardial infarction, cardiac hypertrophy, congestive heart failure, transplant rejection, an intestinal disorder, a neurodegenerative disease, multiple sclerosis, Alzheimer's disease, Parkinson's disease, epilepsy, Huntington's disease, Tourette's syndrome, cerebral ischaemia, cerebral apoplexy, craniocerebral trauma, stroke, spinal cord injury, catabolism, hypotension, hemorrhagic hypotension, endotoxin-induced hypotension, an eye disorder, glaucoma, uveitis, retinopathy, dry eye, macular degeneration, emesis, nausea, a gastric ulcer, diarrhea, pain, a neuropathic pain disorder, viral encephalitis, plaque sclerosis, cancer, a bone disorder, bone density loss, a lung disorder, asthma, pleurisy, polycystic ovary disease, premature abortion; inflammatory bowel disease, lupus, graft vs. host disease, T-cell mediated hypersensitivity disease, Hashimoto's thyroiditis, Guillain-Barre syndrome, contact dermatitis, allergic rhinitis, ischemic injury, and reperfusion injury. In one embodiment, the therapeutically effective amount of a compound of Formula (I) is in a sufficient amount to ameliorate or prevent said disease or condition by binding to a cannabinoid receptor (e.g., CB-1 receptor). In another embodiment, the method can further include identifying a subject in need of ameliorating or preventing said disease or condition.

Also disclosed herein are methods of treating clinical manifestations in which a subject would benefit from modulation of the cannabinoid receptor (e.g., CB-1 receptor), for example, antagonism of or inverse agonism of the cannabinoid receptor (e.g., CB-1 receptor) wherein such modulation would treat clinical manifestations such as obesity, metabolic syndrome, a metabolic disorder, hypertension, polycystic ovary disease, osteoarthritis, a dermatological disorder, hypertension, insulin resistance, hypercholesterolemia, hypertriglyceridemia, cholelithiasis, a sleep disorder, hyperlipidemic conditions, bulimia nervosa, a compulsive eating disorder, an appetite disorder, atherosclerosis, diabetes, high cholesterol, hyperlipidemia, cachexia, an inflammatory disease, rheumatoid arthritis, a neurological disorder, a psychiatric disorder, substance abuse (e.g., alcohol, amphetamines, amphetamine-like substances, caffeine, cannabis, cocaine, hallucinogens, inhalents, nicotine, opioids, phencyclidine, phencyclidine-like compounds, sedative-hypnotics or benzodiazepines, and/or other unknown substances), depression, anxiety, mania, schizophrenia, dementia, dystonia, muscle spasticity, tremor, psychosis, an attention deficit disorder, a memory disorder, a cognitive disorder, short term memory loss, memory impairment (e.g., associated with dementia, Alzheimer's disease, schizophrenia, Parkinson's disease, Huntington's disease, Pick's disease, Creutzfeld-Jakob disease, HIV, cardiovascular disease, head trauma and/or age-related cognitive decline), drug addiction, alcohol addiction, nicotine addiction, infertility, hemorrhagic shock, septic shock, cirrhosis, a cardiovascular disorder, cardiac dysfunction, valvular disease, myocardial infarction, cardiac hypertrophy, congestive heart failure, transplant rejection, an intestinal disorder, a neurodegenerative disease, multiple sclerosis, Alzheimer's disease, Parkinson's disease, epilepsy, Huntington's disease, Tourette's syndrome, cerebral ischaemia, cerebral apoplexy, craniocerebral trauma, stroke, spinal cord injury, catabolism, hypotension, hemorrhagic hypotension, endotoxin-induced hypotension, an eye disorder, glaucoma, uveitis, retinopathy, dry eye, macular degeneration, emesis, nausea, a gastric ulcer, diarrhea, pain, a neuropathic pain disorder, viral encephalitis, plaque sclerosis, cancer, a bone disorder, bone density loss, a lung disorder, asthma, pleurisy, polycystic ovary disease, premature abortion; inflammatory bowel disease, lupus, graft vs. host disease, T-cell mediated hypersensitivity disease, Hashimoto's thyroiditis, Guillain-Barre syndrome, contact dermatitis, allergic rhinitis, ischemic injury, and reperfusion injury, comprising administering to a subject a pharmaceutically effective amount of a compound of Formula I. These methods include, but are not limited to methods such as a method of treating clinical manifestations in which cannabinoid receptor function is altered.

Some embodiments disclosed herein relate to a method for treating or preventing a disease or condition in which it would be beneficial to modulate the activity of a cannabinoid receptor, such as a CB1 receptor, that can include administering a therapeutically effective amount of a compound of Formula I.

In certain embodiments, the neurological disorder can be schizophrenia, dementia, dystonia, muscle spasticity, tremor, psychosis, anxiety, depression, an attention deficit disorder, a memory disorder, a cognitive disorder, drug addiction, alcohol addiction, nicotine addiction, a neurodegenerative disease, multiple sclerosis, Alzheimer's disease, Parkinson's disease, epilepsy, Huntington's disease, Tourette's syndrome, cerebral ischaemia, cerebral apoplexy, craniocerebral trauma, stroke, spinal cord injury, pain, neuropathic pain disorder, viral encephalitis, and/or plaque sclerosis.

In some embodiments, the disease or condition can be obesity, metabolic syndrome, appetite disorders, cachexia, high cholesterol, hyperlipidemia and/or diabetes.

In certain embodiments, the disease or condition can be of the gastrointestinal system such as emesis, nausea, gastric ulcers, diarrhea or intestinal disorders.

In some embodiments, the disease or disorder can be an inflammation disease (e.g., rheumatoid arthritis, asthma, psoriasis).

In certain embodiments, the disease or condition can be of the cardiovascular system such as hemorrhagic sock, septic shock, cirrhosis, atherosclerosis, and/or cardiovascular disorders.

In other embodiments, the disease or condition can be of the reproductive system such as infertility and/or premature abortion.

In some embodiments, the disease or condition can be of the visual system such as glaucoma, uveitis, retinopathy, dry eye and/or macular degeneration.

In certain embodiments, the disease or condition can be osteoporosis and/or ostepenia.

In other embodiments, the disease or condition can be asthma and/or pleurisy.

In certain embodiments, the disease or condition can be cancer.

Another embodiment described herein relates to a method of ameliorating and/or preventing drug and/or alcohol addiction comprising administering to a subject a pharmaceutically effective amount of a compound of Formula (I).

Still another embodiment described herein relates to a method of ameliorating and/or preventing obesity, comprising administering to a subject a pharmaceutically effective amount of a compound of Formula (I).

Yet still another embodiment described herein relates to a method of ameliorating and/or preventing impaired cognition and/or a memory disorder comprising administering to a subject a pharmaceutically effective amount of a compound of Formula (I).

One embodiment described herein relates to a method of improving cognition or memory in a subject comprising administering to a subject a pharmaceutically effective amount of a compound of Formula (I)

Another embodiment described herein relates to a method of ameliorating and/or preventing inflammation due to an inflammatory disease comprising administering to a subject a pharmaceutically effective amount of a compound of Formula (I). A non-limiting list of inflammatory diseases include rheumatoid arthritis, asthma, and psoriasis.

Some embodiment disclosed herein relate to a method of modulating or specifically inverse agonizing or antagonizing a cannabinoid receptor in a subject that includes administering to the subject an effective amount of a compound of Formula I. In one embodiment, the cannabinoid receptor can be a CB1 receptor.

Other embodiments disclosed herein relate to a method of modulating or specifically inverse agonizing or antagonizing a cannabinoid receptor comprising contacting a cannabinoid receptor with a compound of Formula I. In one embodiment, the cannabinoid receptor can be a CB1 receptor.

Still other embodiments disclosed herein relate to a method of modulating or specifically inverse agonizing or antagonizing one or more cannabinoid receptors comprising identifying a subject in need of treatment or prevention and administering to the subject a pharmaceutically effective amount of a compound of Formula I.

Yet still other embodiments disclosed herein relate to a method of identifying a compound which is an agonist, inverse agonist, or antagonist of a cannabinoid receptor that includes contacting a cannabinoid receptor with at least one test compound of Formula I; and determining any increase or decrease in activity level of the cannabinoid receptor so as to identify said test compound as an agonist, inverse agonist or antagonist of the cannabinoid receptor. In one embodiment, the cannabinoid receptor can be a CB1 receptor. In another embodiment, the cannabinoid receptor can consists essentially of SEQ ID NO: 2. In yet still another embodiment, the cannabinoid receptor can have at least 90% amino acid identity to SEQ ID NO: 2. In one embodiment, the cannabinoid receptor can have at least 85% amino acid identity to SEQ ID NO: 2. In another embodiment, the cannabinoid receptor can have at least 70% amino acid identity to SEQ ID NO: 2.

One embodiment disclosed herein relates to a method of identifying a compound which is an agonist, inverse agonist, or antagonist of a cannabinoid receptor that includes culturing cells that express a cannabinoid receptor; incubating the cells or a component extracted from the cells with at least one test compound of Formula I; and determining any increase or decrease in activity of the cannabinoid receptor so as to identify said test compound as an agonist, inverse agonist, or antagonist of the cannabinoid receptor. In one embodiment, the cannabinoid receptor can be a CB1 receptor. In another embodiment, the cannabinoid receptor can consists essentially of SEQ ID NO: 2. In yet still another embodiment, the cannabinoid receptor can have at least 90% amino acid identity to SEQ ID NO: 2. In one embodiment, the cannabinoid receptor can have at least 85% amino acid identity to SEQ ID NO: 2. In another embodiment, the cannabinoid receptor can have at least 70% amino acid identity to SEQ ID NO: 2.

Another embodiment disclosed herein relates to a method for identifying a compound which binds to a cannabinoid receptor that includes labeling a compound of Formula I with a detectable label; and determining the number of occupied cannabinoid receptors. In one embodiment, the detectable label can be a radiolabel (e.g, [³H]).

Any of the embodiments listed herein may further include identifying a subject in need of treatment or ameliorating of any disease or condition identified herein.

Other embodiments disclosed herein relate to a method of identifying a compound that treats or amerliorates any disease or condition identified herein in a subject, comprising identifying a subject suffering the disease or condition; providing the subject with at least one compound of Formula I, as defined herein; and determining if the at least one compound treats the disease or condition in the subject.

Pharmaceutical Compositions

In another aspect, the present invention relates to a pharmaceutical composition comprising a compound of Formula I as described above, and a physiologically acceptable carrier, diluent, or excipient, or a combination thereof.

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, intramuscular, intraocular, intranasal, intravenous, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical compositions will generally be tailored to the specific intended route of administration.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990, which is hereby incorporated by reference in its entirety.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, intraocular injections or as an aerosol inhalant.

Alternately, one may administer the compound in a local rather than systemic manner, for example, via injection of the compound directly into the area of pain or inflammation, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes.

Pharmaceutical compositions for use in accordance with the present disclosure thus may be formulated in conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., as disclosed in Remington's Pharmaceutical Sciences, cited above.

For injection, the agents disclosed herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethy-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An exemplary pharmaceutical carrier for the hydrophobic compounds disclosed herein is a co-solvent system comprising benzyl alcohol, a nonpolar surfactant, a water-miscible organic polymer, and an aqueous phase. A common co-solvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80™; the fraction size of polyethylene glycol may be varied; and other biocompatible polymers may replace polyethylene glycol, e.g., polyvinyl pyrrolidone. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acids or base forms.

Pharmaceutical compositions suitable for use in the methods disclosed herein include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the patient's condition. (See e.g., Fingl et al. 1975, in “The Pharmacological Basis of Therapeutics”, Chapter 1, which is hereby incorporated by reference in its entirety). Typically, the dose range of the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Where no human dosage is established, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions disclosed herein or a pharmaceutically acceptable salt thereof calculated as the free base, the composition being administered 1 to 4 times per day. Alternatively the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. In some embodiments, the compounds will be administered for a period of continuous therapy, for example for a week or more, or for months or years.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### EXAMPLES

Embodiments of the present invention are disclosed in further detail in the following examples, which are not in any way intended to limit the scope of the invention.

### Example 1

### General Analytical LC-MS Procedure

Procedure 1 (AP1): The analysis was performed on a combined prep/analytical Waters/Micromass system consisting of a ZMD single quadropole mass spectrometer equipped with electro-spray ionization interface. The HPLC system consisted of a Waters 600 gradient pump with on-line degassing, a 2700 sample manager and a 996 PDA detector.

Separation was performed on an X-Terra MS C18, 5 μm 4.6×50 mm column. Buffer A: 10 mM ammonium acetate in water, buffer B: 10 mM ammonium acetate in acetonitrile/water 95/5. A gradient was run from 30% B to 100% B in 10 min, dwelling at 100% B for 1 min, and re-equilibrating for 6 min. The system was operated at 1 ml/min.

Procedure 2 (AP2): The analysis was performed on a combined prep/analytical Waters/Micromass system consisting of a ZMD single quadropole mass spectrometer equipped with electro-spray ionization interface. The HPLC system consisted of a Waters 600 gradient pump with on-line degassing, a 2700 sample manager and a 996 PDA detector.

Separation was performed on an X-Terra MS C18, 5 μm 4.6×50 mm column. Buffer A: 10 mM ammonium acetate in water, buffer B: 10 mM ammonium acetate in acetonitrile/water 95/5. A gradient was run from 30% B to 100% B in 7 min, dwelling at 100% B for 1 min, and re-equilibrating for 5.5 min. The system was operated at 1 ml/min.

### Example 2

### General Gas Chromatography (GC) Procedure

GC method 50 was used. Method 50 starts at 50° C. and has a gradient of 20° C./min until 250° C. then holds the temperature for 5 minutes. The analysis was performed on an Aglient 6850 series GC system with capillary S/SL inlet and FID with EPC installation. The column was a 30 m×0.32 mm×0.25 μm HP5 column.

### Example 3

### 4-(2-methoxycarbonyl-phenylsulfanyl)-3-nitro-benzoic acid ethyl ester

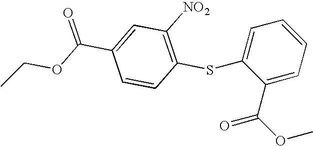

Methyl 2-mercaptobenzoate (4.67 ml, 34 mmol) was added during 30 min to a mixture of ethyl 4-flouro-3-nitrobenzoate (6.60 g, 30.9 mmol) and Cs₂CO₃ (10.06 g, 30.9 mol) in DMF (60 mL) at 40° C. The reaction mixture was diluted with EtOAc, water after additional 15 min (full conversion according to TLC). The aqueous phase was extracted once with EtOAc and the combined organic phases were washed twice with water followed by brine and then dried (Na₂SO₄). Filtration and concentration of the organic phase at reduce pressure gave a yellow crystalline residue. Recrystallization from EtOAc/heptane gave 10.3 g (92%) of the titled compound as yellow crystals. ¹H NMR (400 MHz, CDCl₃) δ 8.82 (d, 1H, J=1.9 Hz), 7.94 (m, 2H), 7.62-7.57 (m, 3H), 6.92 (d, 1H, J=8.6 Hz), 4.38 (q, 2H, J=7.2 Hz), 3.78 (s, 3H), 1.38 (t, 3H, J=7.0 Hz); ¹³C NMR (100 MHz, CDCl₃); δ 166.8, 164, 145.5, 144.1, 137.6, 136.3, 133.4, 133.0, 131.5, 131.3, 130.5, 129.8, 128.1, 126.9, 61.9, 52.7, 14.5.

### Example 4

### 4-(2-carboxy-phenylsulfanyl)-3-nitro-benzoic acid

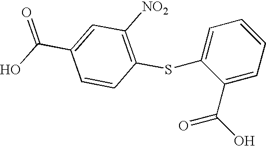

4-(2-methoxycarbonyl-phenylsulfanyl)-3-nitro-benzoic acid ethyl ester (9.56 g, 26.5 mmol) dissolved in THF (570 mL) and aqueous LiOH (264 ml, 1M) was stirred at 60° C. for 2 h, then allowed to cool to room temperature. THF was removed at reduced pressure and the remaining aqueous mixture was extracted once with EtOAc. HCl (2M) was then added to the resulting aqueous solution until pH 2. The precipitation was filtred off, washed with water and finally dried, which afforded 8.7 g (99%) of the titled compound as yellow crystals. The crude product was sufficiently pure to be used in the next step without further purifications. ¹H NMR (400 MHz, CD₃OD) δ 8.71 (d, 1H, J=1.8 Hz), 7.95 (m, 2H), 7.64-7.59 (m, 3H), 7.00 (d, 1H, J=8.6 Hz); ¹³C NMR (100 MHz, CD₃OD) δ 168.3, 166.1, 145.9, 143.3, 137.0, 136.5, 133.2, 132.6, 131.2, 131.1, 130.1, 130.0, 128.6, 126.3.

### Example 5

### 3-Amino-4-(2-carboxy-phenylsulfanyl)-benzoic acid

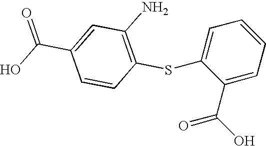

Pd/C (10%, 200 mg) and PtO₂ were added to 4-(2-carboxy-phenylsulfanyl)-3-nitro-benzoic acid (2.9 g, 9.1 mmol) dissolved in 100 ml of MeOH. The reaction flask were repeatedly evacuated and filled with H₂. A balloon containing H₂ was connected to the flask. After 16 h the reaction mixture was filtered through a pad of celite, which was then washed carefully with MeOH. Concentration of the filtrate at reduced pressure gave 2.5 g (96% yield, approximately 95% purity) of the titled compound as a white solid. The purity could be increased to 97% by recrystallization from EtOAc/MeOH (2.3 g, 88% yield). ¹H NMR (400 MHz, CD₃OD) δ 8.01 (d, 1H, J=7.6 Hz), 7.51 (s, 1H), 7.44 (d, 1H, J=8.0 Hz), 7.31 (d, 1H, J=8.0 Hz), 7.28 (t, 1H, J=8.0 Hz), 7.16 (t, 1H), J=7.2 Hz), 6.74 (d, 1H, J=8.0 Hz); MS (ES⁺, M+1)=290.

### Example 6

### 11-Oxo-10,11-dihydro-dibenzo[b,f] [1,4]thiazepine-8-carboxylic acid

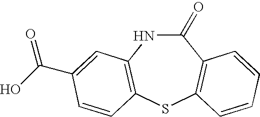

CDI (4.53 g, 29 mmol, 4 eq) was added to 3-Amino-4-(2-carboxy-phenylsulfanyl)-benzoic acid (2.1 g, 7.3 mmol) dissolved in THF (30 ml). The reaction was stirred for 16 h at room temperature. Water (200 ml) was then added to the mixture resulting in, after filtration and drying, 1.78 g (91%) of the titled compound as a off-white solid. ¹H NMR (400 MHz, DMSO-d₆) δ 10.78 (br s, 1H), 7.77 (s, 1H), 7.67 (m, 3H), 7.55-7.42 (m, 3H); ¹³C NMR (100 MHz, DMSO-d₆); δ 168.9, 166.9, 140.3, 138.3, 136.0, 134.5, 133.5, 133.0, 132.9, 132.2, 132.1, 129.9, 126.5, 124.3.

### Example 7

### 11-Chloro-dibenzo[b,f] [1,4]thiazepine-8-carbonyl chloride

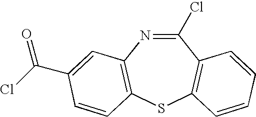

A solution of 11-Oxo-10,11-dihydro-dibenzo[b,f] [1,4]thiazepine-8-carboxylic acid_ (200 mg, 0.74 mmol) and phosphorus pentachloride (756 mg, 3.68 mmol) in 4 mL toluene was heated to 110° C. for 2 h. Toluene and excess of phosphorus pentachloride was removed at reduced pressure to give the title compound (193 mg, 85%) as an yellow solid. ¹H NMR (400 MHz, CDCl₃) δ 8.01 (d, 1H, J=2.0 Hz), 7.87 (dd, 1H, J=8.4, 2.2 Hz), 7.77 (m, 1H), 7.58 (d, 1H, J=8.2 Hz), 7.47-7.44 (m, 2H), 7.44-7.39 (m, 1H); ¹³C NMR (100 MHz, CDCl₃); δ 167.5, 157.1, 146.7, 137.8, 137.4, 136.3, 134.5, 133.4, 133.3, 132.6, 130.3, 129.5, 129.1, 128.8;

### Example 7b

### Alternative synthesis of 11-Chloro-dibenzo[b,f] [1,4]thiazepine-8-carbonyl chloride

A solution of SOCl₂ (25 ml), 11-Oxo-10,11-dihydro-dibenzo[b,f] [1,4]thiazepine-8-carboxylic acid (1.24 g, 4.6 mmol) and DMF (0.05 ml) in toluene (25 ml) was heated at 80° C. for 17 h. Toluene and excess SOCl₂ were removed at reduced pressure to give 1.18 g (84%) of the title compound5 as a yellow solid, which was used in the next step without further purifications. ¹H NMR (400 MHz CDCl₃) δ 8.01 (d, 1H, j=2.0 Hz), 7.87 (dd, 1H, J=8.4, 2.2 Hz), 7.77 (m, 1H), 7.58 (d, 1H, J=8.2 Hz), 7.47-7.44 (m, 2H), 7.44-7.39 (m, 1H); ¹³C NMR (100 MHz, CDCl₃); δ 167.5, 157.1, 146.7, 137.8, 137.4, 136.3, 134.5, 133.4, 133.3, 130.3, 129.5, 129.1, 128.8.

### Example 8

### N-(butyl)-11-(chloro)-dibenzo[b,f,] [1,4thiazepine-8-carboxamide

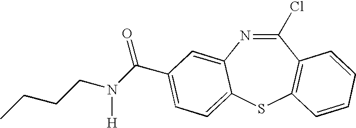

11-Chloro-dibenzo[b,f] [1,4]thiazepine-8-carbonyl chloride (616 mg; 2 mmol) dissolved in dry DCM (5 mL) was added to a solution of butylamine (366 mg; 5 mmol) in dry DCM (10 mL) was added at 0° C. The reaction was stirred for 30 min and then diluted with EtOAc. The organic phase was washed with NH₄Cl (aq), brine and dried (Na₂SO₄). Filtration and evaporation at reduced pressure followed by purification by column chromatography (ethyl acetate/heptane 1:1) gave the title compound (557 mg, 81%) as a yellow solid. MS (ES⁺, M+1)=345.

### Example 9

### N-(butyl)-1-(4-chlorophenyl)-dibenzo[b,f,] [1,4]thiazepine-8-carboxamide

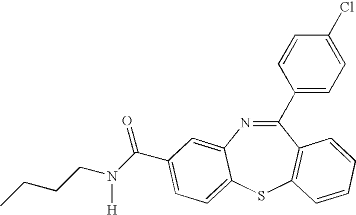

4-Chlorophenylzinc iodide (0.5M in THF, 35 mL) was added to N-(butyl)-11-(chloro)-dibenzo[b,f,] [1,4]thiazepine-8-carboxamide (2.8 g; 8.1 mmol) and PdCl₂(PPh₃)₂ (5 mol %, 275 mg) in dry THF (90 mL) at room temperature. After 3 h saturated aqueous NH₄Cl and EtOAc was added and the aqueous phase was extracted twice with EtOAc. The combined organic phases were washed with brine and then dried (Na₂SO₄). Filtration, concentration at reduced pressure of the organic phase followed by purification by column chromatography (heptane/EtOAc 3:1 to 1:1) and recrystallization from toluene gave 2.86 g (84%) of the title compound as pale yellow crystals. m.p. 217-219° C. ¹H NMR (400 MHz, CDCl₃) δ 7.75 (m, 2H), 7.64 (d, 1H, J=1.2 Hz), 7.55 (dd, 1H, J=7.8, 1.2 Hz), 7.50 (m, 2H), 7.42 (m, 3H), 7.31 (dt, 1H, J=7.6, 1.2 Hz), 7.16 (dd, 1H, J=7.6, 1.4 Hz). 6.06 (br s, 1H), 3.44 (q, 2H, J=7.2 Hz). 1.58 (m, 2H), 1.40 (m, 2H, J=7.4 Hz), 0.95 (t, 3H, J=7.2 Hz); MS (ES⁺, M+1)=421.

### Example 10

### 11-Chloro-dibenzo[b,f] [1,4]thiazepine-8-carboxylic acid isobutylamide

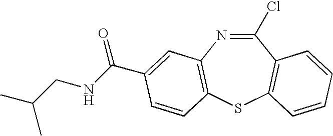

A solution of 11-chloro-dibenzo[b,f] [1,4]thiazepine-8-carbonyl chloride (0.59 g; 1.92 mmol) in DCM (10 mL) was added to a solution of isobutylamine (0.38 mL; 3.84 mmol) in DCM (10 mL) at 0° C. under argon. The mixture was stirred at room temperature for 1/2 hour. The reaction mixture was diluted with DCM and NH₄Cl (sat). The aqueous phase was extracted twice with DCM and the combined organic phases dried over Na₂SO₄. After filtration and concentration by evaporation, the residue was purified by silica gel column chromatography eluting with 10-20% EtOAc in n-heptane. 0.51 g (77%) of the title compound was obtained as a white powder.

¹H NMR (400 MHz, CDCl₃) δ 7.77-7.73 (m, 1H, ArH), 7.63 (dd, 1H, J=2.0, 8.0, ArH), 7.56 (d, 1H, J=2.0, ArH), 7.51 (d, 1H, J=8.0, ArH), 7.47-7.37 (m, 3H, ArH), 6.07 (br s, 1H, NH), 3.26 (dd, 2H, J=6.1, 6.8, CH_{2iBu}), 1.86 (sept, 1H, J=6.6, CH_{iBU}), 0.96 (d, 6H, J=6.6, 2×CH₃).

### Example 11

### 11-(5-Chlorothiophen-2-yl)dibenzo[b,f] [1,4]thiazepine-8-carboxylic acid isobutylamide

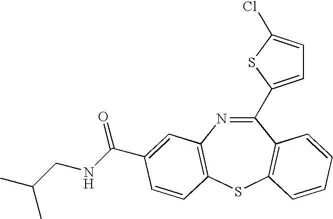

5-Chloro-2-thienyl zinc bromide (0.5 M in THF, 3.5 mL; 1.72 mmol) was added to a solution of 11-chloro-dibenzo[b,f] [1,4]thiazepine-8-carboxylic acid isobutylamide (0.15 g; 0.43 mmol) and bis(triphenylphosphine)palladium(II)chloride (30 mg; 0.043 mmol) in 4 mL dry THF at room temperature. The mixture was stirred overnight at room temperature. The reaction mixture was partitioned between EtOAc and NH4Cl (sat). The organic layer was dried over Na2SO4, filtered and evaporated to dryness. The mixture was purified by silica gel column chromatography (10-30% EtOAc in n-heptane) and repurified by prep HPLC to afford the title compound as a yellow solid (27 mg; 15%).

¹H NMR (400 MHz, CDCl₃) δ 7.60-7.34 (m, 7H, ArH), 6.94 (d, 1H, J=4.0, thiophenH), 6.89 (d, 1H, J=4.0, thiopheneH), 6.15 (br m, 1H, NH), 3.26 (dd, 2H, J=6.4, 7.2, CH_{2iBu}), 1.87 (m, 1H, CH_{iBu}), 0.96 (d, 6H, J=6.8, 2×CH₃). ¹³C NMR (100 MHz, CDCl₃) δ 166.8, 162.7, 148.5, 145.1, 140.5, 137.1, 136.2, 135.3, 133.0, 132.8, 132.1, 132.0, 131.9, 130.3, 128.4, 127.3, 124.7, 123.9, 47.6, 28.8, 20.4. MS (ES⁺, M+1)=427.

General Procedure A—Amide Formation:

A flame-dried flask was charged under argon with 11-Chloro-dibenzo[b,f] [1,4]thiazepine-8-carbonyl chloride (180 mg; 0.58 mmol) in 4 mL dry DCM and cooled to 0° C. The amine (1.45 mmol) was then slowly added and the reaction was allowed to reach room temperature and stirred for 30 min. The reaction was diluted with DCM and the organic phase was washed with NH₄Cl (aq), brine and dried (Na₂SO₄). Filtration and evaporation at reduced pressure follwed by purification by column chromatography (ethyl acetate/heptane 1:1) gave the compounds listed as Examples 12-14 (72-88%) as off-white solids.

### Example 12

### (11-chloro-dibenzo[b,f] [1,4]thiazepin-8-yl)-[2,4-dimethyl-phenyl)-piperazin-1-yl]-methanone

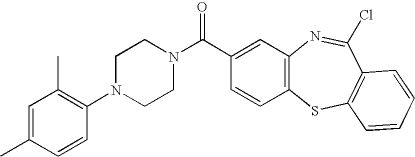

The reaction was performed according to the general procedure A, which gave 220 mg (82%) of the titled compound. ¹H NMR (400 MHz, CDCl₃) δ 7.75 (m, 1H), 7.51 (d, 1H, J=8.0 Hz), 7.47-7.44 (m, 2H), 7.44-7.39 (m, 1H), 7.31, (d, 1H, J=1.8 Hz), 7.24 (dd, 1H, J=7.8, 1.8 Hz), 7.02 (br s, 1H), 6.98 (br d, 1H, J=8.0 Hz), 6.89 (d, 1H, J=8.0 Hz), 3.88 (br s, 2H), 3.54 (br s, 2H), 2.85 (br s, 4H), 2.28 (s, 6H); ¹³C NMR (100 MHz, CDCl₃) δ 169.0, 156.2, 148.5, 146.4, 138.4, 137.9, 137.6, 133.6, 133.3, 133.1, 132.9, 132.3, 132.1, 130.2, 129.5, 129.1, 127.4, 126.1, 124.3, 119.4, 31.1, 20.9, 17.8; MS (ES⁺, M)=462.

### Example 13

### 11-chloro-dibenzo[b,f] [1,4]thiazepin-8-carboxylic acid piperidin-1-ylamide

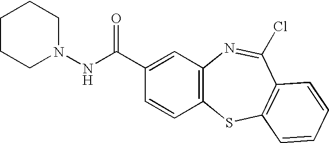

The reaction was performed according to the general procedure A, which gave 157 mg (72%) of the titled compound. ¹H NMR (400 MHz, CDCl₃) δ 7.74 (m, 1H), 7.59 (dd, 1H, J=8.0, 1.8 Hz), 7.54 (s, 1H), 7.50 (d, 1H, J=8.2 Hz), 7.47-7.43 (m, 2H), 7.43-7.39 (m, 1H), 2.80 (br s, 4H), 1.74 (br s, 4H), 1.44 (br s, 2H); ¹³C NMR (100 MHz, CDCl₃) δ 164.3, 156.6, 146.5, 138.5, 138.1, 135.8, 133.5, 133.4, 132.7, 131.8, 130.4, 129.4, 126.7, 124.2, 57.7, 32.4, 25.8; MS (ES⁺, M+1)=372.

### Example 14

### 4-[(11-chloro-dibenzo[b,f] [1,4]thiazepine-8-carbonyl)-amino]-piperidine-1-carboxylic acid ethyl ester

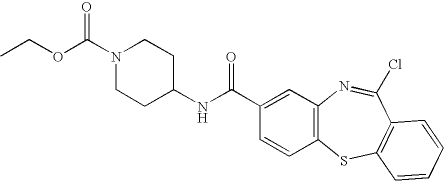

The reaction was performed according to the general procedure A, which gave 189 mg (88%) of the titled compound. ¹H NMR (400 MHz, CDCl₃) δ 7.74 (m, 1H), 7.61 (dd, 1H, J=8.2, 1.9 Hz), 7.56 (d, 1H, J=1.6Hz), 7.51 (d, 1H, J=8.2 Hz), 7.47-7.44 (m, 2H), 7.44-7.39 (m, 1H), 6.00 (d, 1H, J=7.6Hz), 4.12 (m, 5H), 2.94 (t, 2H, J=11.9 Hz), 2.00 (m, 2H), 1.38 (m, 2H), 1.26 (dt, 3H, J=7.2, 1.6 Hz); ¹³C NMR (100 MHz, CDCl₃) δ 165.6, 156.3, 155.7, 146.3, 138.3, 137.8, 136.0, 133.2, 133.2, 132.4, 131.6, 130.2, 129.1, 126.2, 123.9, 61.7, 47.5, 43.0, 32.2, 14.9; MS (ES⁺, M+1)=444.

General Procedure B—Iron-Catalyzed Alkyl-Imidoyl Chloride Cross-Coupling

A flame-dried flask was charged under argon with the imidoyl chloride (0.05 mmol), Fe(acac)₃ (0.9 mg, 0.0025 mmol), THF (1 mL) and NMP (0.1 mL). A solution of alkylmagnesium halogen (2M in Et₂O, 100 μL, 0.20 mmol) was slowly added to the resulting red solution, causing an immediate colour change to dark brown. The resulting mixture was stirred for 10 min, and the reaction was then carefully quenched with NH₄Cl (aq) and diluted with Et₂O. The organic phase was washed with brine, dried (Na₂SO₄), filtered and evaporated to give the crude product. Purification by column chromatography (ethyl acetate/heptane/MeOH 1:1:0.05) gave the product (60-90%).

### Example 15

### (11-Butyl-dibenzo[b,f] [1,4]thiazepin-8-yl)-[4-(2,4-Dimethyl-phenyl)-piperazin-1-yl]methanone.

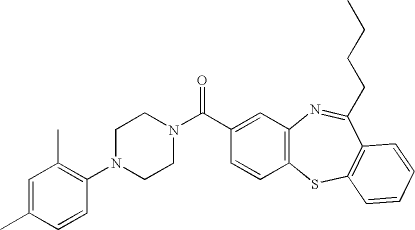

The reaction was performed according to the general procedure B, which gave 18.7 mg (77%) of the titled compound. ¹H NMR (400 MHz, CDCl₃) δ 7.45 (m, 2H), 7.40-7.32 (m, 3H), 7.23 (d, 1H, J=1.8 Hz), 7.08 (dd, 1H, J=8.0, 1.8 Hz), 7.02 (br s, 1H), 6.98 (br d, 1H, J=8.0 Hz), 6.89 (d, 1H, J=8.0 Hz), 3.88 (br s, 2H), 3.58 (br s, 2H), 3.05-2.75 (m, 6H), 2.29 (s, 6H), 1.7 (m, 2H), 1.5 (m, 2H), 0.95 (t, 3H, J=7.4 Hz); ¹³C NMR (100 MHz, CDCl₃) δ 174.6, 169.7, 149.1, 148.6, 140.0, 139.0, 137.0, 133.5, 132.9, 132.8, 132.1, 130.8, 130.6, 128.8, 127.9, 127.4, 123.8, 123.8, 119.4, 42.3, 29.6, 22.7, 20.9, 17.8, 14.2; MS (ES⁺, M+1)=484.

### Example 16

### [4-(2,4-Dimethyl-phenyl)-piperazin-1-yl]-(11-pentyl-dibenzo[b,f] [1,4]thiazepin-8-yl)methanone

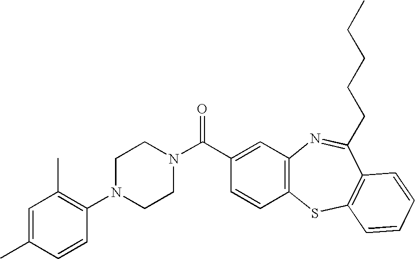

The reaction was performed according to the general procedure B, which gave 20.1 mg (81%) of the titled compound. ¹H NMR (400 MHz, CDCl₃) δ 7.46 (m, 2H), 7.40-7.32 (m, 3H), 7.23 (d, 1H, J=1.6 Hz), 7.08 (dd, 1H, J=8.0, 1.8 Hz), 7.02 (br s, 1H), 6.98 (br d, 1H, J=8.0 Hz), 6.89 (d, 1H, J=8.0 Hz), 3.88 (br s, 2H), 3.58 (br s, 2H), 3.05-2.75 (m, 6H), 2.29 (s, 6H), 1.7 (m, 2H), 1.5-1.2 (m, 4H), 0.95 (t, 3H, J=7.0 Hz); ¹³C NMR (100 MHz, CDCl₃) δ 174.9, 170.0, 149.3, 148.9, 140.3, 139.3, 137.3, 133.8, 133.1, 133.1, 130.9, 129.0, 128.2, 127.6, 124.1, 119.7, 42.7, 32.0, 27.3, 22.9, 21.2, 18.1, 14.5; MS (ES⁺, M+1)=498.

### Example 17

### [4-(2,4-Dimethyl-phenyl)-piperazin-1-yl]-(11-isobutyl-dibenzo[b,f] [1,4]thiazepin-8-yl) methanone

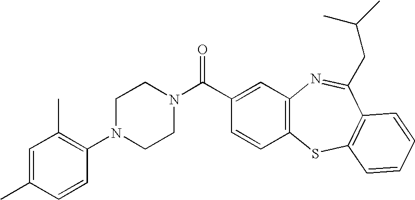

The reaction was performed according to the general procedure B, which gave 17.3 mg (72%) of the titled compound. MS (ES⁺, M+1)=484.

### Example 18

### (11-Cyclohexyl-dibenzo[b,f] [1,4]thiazepin-8-yl)-[4-(2,4-dimethyl-phenyl)-piperazin-1-yl]methanone

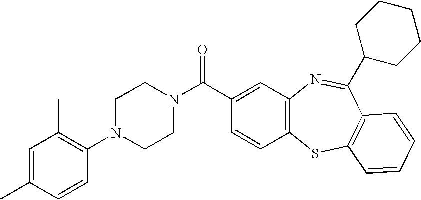

The reaction was performed according to the general procedure B, which gave 16.8 mg (66%) of the titled compound. MS (ES⁺, M+1)=510

### Example 19

### [11-(4-chloro-phenyl)-dibenzo[b,f] [1,4]thiazepin-8-yl)]-[4-(2,4-dimethyl-phenyl)-piperzin-1-yl]-methanone

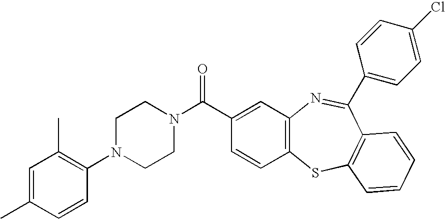

The reaction was performed according to the general procedure B, which gave 16.2 mg (60%) of the titled compound. MS (ES⁺, M)=538.

### Example 20

### 11-Propyl-dibenzo[b,f] [1,4]thiazepine-8-carboxylic acid piperidin-1-ylamide

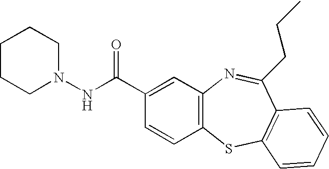

The reaction was performed according to the general procedure B, which gave 15.3 mg (81%) of the titled compound. MS (ES⁺, M+1)=380.

### Example 21

### 11-Butyl-dibenzo[b,f] [1,4]thiazepine-8-carboxylic acid piperidin-1-ylamide

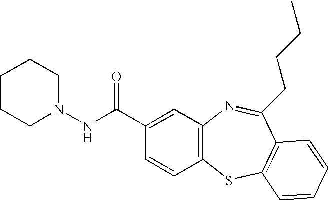

The reaction was performed according to the general procedure B, which gave 15.8 mg (80%) of the titled compound. MS (ES⁺, M+1)=394.

### Example 22

### 11-Pentyl-dibenzo[b,f] [1,4]thiazepine-8-carboxylic acid piperidin-1-ylamide

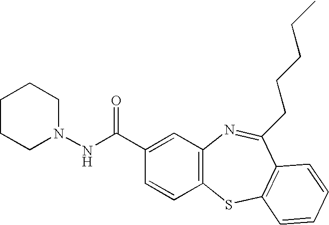

The reaction was performed according to the general procedure B, which gave 16.1 mg (79%) of the titled compound. MS (ES⁺, M+1)=408.

### Example 23

### 11-Isobutyl-dibenzo[b,f] [1,4]thiazepine-8-carboxylic acid piperidin-1-ylamide

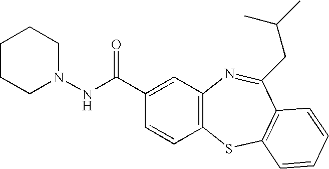

The reaction was performed according to the general procedure B, which gave 16.2 mg (82%) of the titled compound. MS (ES⁺, M+1)=394.

### Example 24

### 11-Cyclohexyl-dibenzo[b,f] [1,4]thiazepine-8-carboxylic acid piperidin-1-ylamide

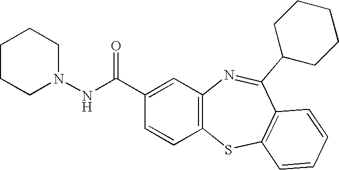

The reaction was performed according to the general procedure B, which gave 15.9 mg (76%) of the titled compound. MS (ES⁺, M+1)=420.

### Example 25

### 4-[(11-Propyl-dibenzo[b,f] [1,4]thiazepine-8-carbonyl)-amino]-piperidine-1-carboxylic acid ethyl ester

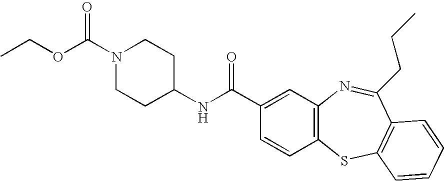

The reaction was performed according to the general procedure B, which gave 19.7 mg (87%) of the titled compound. MS (ES⁺, M+1)=452.

### Example 26

### 4-[(11-Butyl-dibenzo[b,f] [1,4]thiazepine-8-carbonyl)-amino]-piperidine-1-carboxylic acid ethyl ester

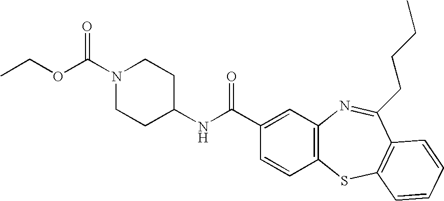

The reaction was performed according to the general procedure B, which gave 19.2 mg (83%) of the titled compound. ¹H NMR (400 MHz, CD3OD) δ 7.45 (dd, 1H, J=1.4, 0.8 Hz), 7.44-7.37 (m, 3H), 7.34-7.28 (m, 3H), 4.03 (q, 2H, J=7.1 Hz), 4.03 (m, 2H), 3.92 (m, 1H), 3.00 (m, 1H), 2.84 (br t, 2H, J=11.9), 2.78 (m, 1H), 1.80 (d, 2H, J=12.5 Hz), 1.52 (m, 2H), 1.37 (m, 4H), 1.15 (t, 3H, J=7.0 Hz), 0.83 (t, 3H, J=7.4 Hz); MS (ES⁺, M+1)=466.

### Example 27

### 4-[(11-Pentyl-dibenzo[b,f] [1,4]thiazepine-8-carbonyl)-amino]-piperidine-1-carboxylic acid ethyl ester.

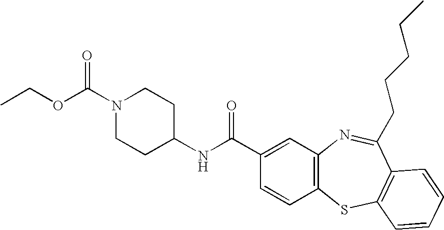

The reaction was performed according to the general procedure B, which gave 20.1 mg (84%) of the titled compound. ¹H NMR (400 MHz, CDCl₃) δ 7.46 (m, 4H), 7.39-7.32 (m, 3H), 5.89 (d, 1H, J=7.6 Hz), 4.12 (q, 2H, J=7.0 Hz), 4.10 (m, 3H), 2.92 (m, 4H), 1.98 (d, 2H, J=11.9 Hz), 1.68 (m, 2H), 1.39 (m, 6H), 1.25 (t, 3H, J=7.1 Hz), 0.90 (t, 3H, J=7.2 Hz); ¹³C NMR (100 MHz, CDCl₃) δ 174.6, 165.9, 155.4, 148.6, 139.6, 138.7, 135.3, 132.6, 132.0, 130.7, 128.6, 127.6, 123.9, 123.0, 61.4, 47.1, 42.7, 42.2, 32.0, 31.4, 26.8, 22.4, 14.6, 13.9; MS (ES⁺, M+1)=480.

### Example 28

### 4-[(11-Isobutyl-dibenzo[b,f] [1,4]thiazepine-8-carbonyl)-amino]-piperidine-1-carboxylic acid ethyl ester

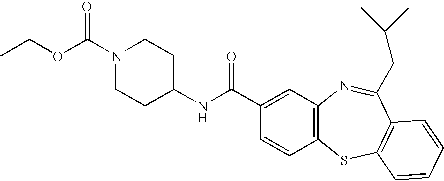

The reaction was performed according to the general procedure B, which gave 17.3 mg (74%) of the titled compound. ¹H NMR (400 MHz, CDCl₃) δ 7.46 (m, 4H), 7.39-7.31 (m, 3H), 5.98 (d, 1H, J=7.8 Hz), 4.12 (q, 2H, J=7.0 Hz), 4.10 (m, 3H), 3.03 (dd, 1H, J=14.1, 5.5 Hz), 2.85 (t, 2H, J=13.7 Hz), 2.63 (dd, 1H, J=14.1, 9.0 Hz), 1.98 (m, 3H), 1.35 (m, 2H), 1.25 (t, 3H, J=7.1 Hz), 1.08 (d, 3H, J=6.5 Hz), 1.03 (d, 3H, J=6.5 Hz); ¹³C NMR (100 MHz, CDCl₃) δ 174.2, 166.2, 155.7, 148.8, 139.8, 139.0, 135.5, 132.9, 132.8, 132.4, 131.0, 128.9, 128.1, 124.3, 123.4, 61.6, 51.7, 47.4, 43.0, 42.2, 32.3, 27.3, 23.4, 22.4, 14.9; MS (ES⁺, M+1)=466.

### Example 29

### 4-[(11-Cyclohexyl-dibenzo[b,f] [1,4]thiazepine-8-carbonyl)-amino]-piperidine-1-carboxylic acid ethyl ester

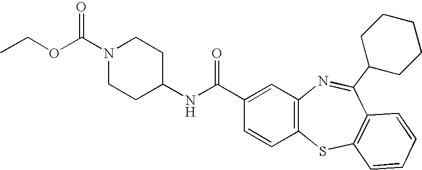

The reaction was performed according to the general procedure B, which gave 21.3 mg (87%) of the titled compound. MS (ES⁺, M+1)=492.

### Example 30

### 4-[(11-(4-chloro-phenyl)-dibenzo[b,f] [1,4]thiazepine-8-carbonyl)-amino]-piperidine-1-carboxylic acid ethyl ester

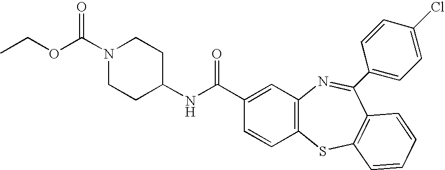

The reaction was performed according to the general procedure B, which gave 18.2 mg (70%) of the titled compound. MS (ES⁺, M)=520.

### Example 30b

### Alternative synthesis of 4-[(11-(4-chloro-phenyl)-dibenzo[b,f] [1,4]thiazepine-8-carbonyl)-amino]-piperidine-1-carboxylic acid ethyl ester

4-chlorophenylzinc iodide (0.5M in THF, 11.5 ml, 5.76 mmol) was added dropwise to 4-[(11-chloro-dibenzo[b,f] [1,4]thiazepine-8-carbonyl)-amino]-piperidine-1-carboxylic acid ethyl ester (640 mg, 1.44 mmol), and PdCl₂(PPh₃)₂ (59 mg, 0.14 mmol, 0.1 eq) in dry THF (15 ml) at room temperature. After 30 min saturated aqueous NH₄Cl and EtOAc was added and the aqueous phase was extracted once with EtOAc. The combined organic phases were washed with water, brine and then dried (Na₂SO₄). Filtration, concentration at reduced pressure of the organic phase followed by purification of the crude product by column chromatography (Heptane-EtOAc-MeOH 1:1:0.01) gave 730 mg (97%) of the titled compound as yellow crystals. ¹H NMR (400 MHz, acetone-d₆) δ 7.82 (d, 2H, J=8.8 Hz), 7.78 (d, 1H, J=2.0 Hz), 7.62 (m, 3H), 7.58-7.52 (m, 4H), 7.45 (dt, 1H, J=8.8, 1.4 Hz), 7.29 (dd, 1H, J=5.8, 1.6 Hz), 4.08 (m, 5H), 2.96 (m, 2H), 1.93 (m, 2H), 1.52 (m, 2H), 1.22 (t, 3H, 7.0 Hz); ¹³C NMR (100 MHz, acetone-d₆) δ 167.8, 165.1, 155.1, 148.7, 140.4, 139.0, 136.9, 136.8, 136.6, 132.4, 132.0, 131.5, 131.3, 130.5, 128.9, 128.7, 124.9, 124.1, 60.8, 47.4,42.9, 31.9, 14.3.

General Procedure C: Palladium Catalyzed Negishi Cross-Coupling of Imidoyl Chlorides and Arylzinc Halides.

The arylzinc halide (3-5 eq) was added to the imidoyl chloride (10 mg) and PdCl₂(PPh₃)₂ (10 mol %) in dry THF (1 ml) at room temperature. After 30 min saturated aqueous NH₄Cl and EtOAc was added and the aqueous phase was extracted once with EtOAc. The combined organic phases were washed with water, brine and then dried (Na₂SO₄). Filtration, concentration at reduced pressure of the organic phase followed by purification of the crude product by column chromatography (Heptane-EtOAc 1:1) gave the product.

### Example 31

### 11-phenyl-dibenzo[b,f] [1,4]thiazepin-8-carboxylic acid piperidin-1-ylamide

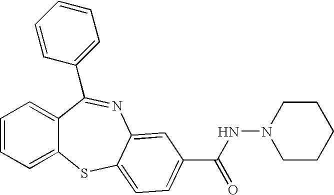

The reaction was performed according to the general procedure C using 11-chloro-dibenzo[b,f] [1,4]thiazepin-8-carboxylic acid piperidin-1-ylamide and phenylzinc iodide, which gave 4.9 mg of the titled compound. MS (ES⁺, M+1 )=414.

### Example 32

### 11-(2-cyanophenyl)-dibenzo[b,f] [1,4]thiazepin-8-carboxylic acid piperidin-1-ylamide

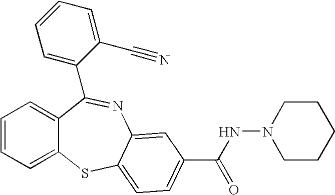

The reaction was performed according to the general procedure C using 11-chloro-dibenzo[b,f] [1,4]thiazepin-8-carboxylic acid piperidin-1-ylamide and 2-cyanophenylzinc iodide, which gave 5.4 mg of the titled compound. MS (ES⁺, M+1)=439.

### Example 33

### 11-(3-bromophenyl)-dibenzo[b,f] [1,4]thiazepin-8-carboxylic acid piperidin-1-ylamide

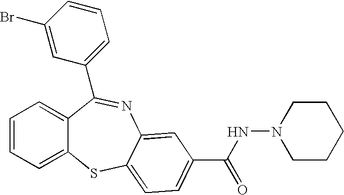

The reaction was performed according to the general procedure C using 11-chloro-dibenzo[b,f] [1,4]thiazepin-8-carboxylic acid piperidin-1-ylamide and 3-bromophenylzinc iodide, which gave 6.4 mg of the titled compound. MS (ES⁺, M+1)=492.

### Example 34

### 11-(4-chlorophenyl)-dibenzo[b,f] [1,4]thiazepin-8-carboxylic acid piperidin-1-ylamide

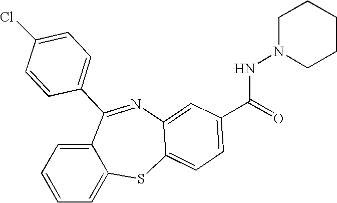

The reaction was performed according to the general procedure C using 11-chloro-dibenzo[b,f] [1,4]thiazepin-8-carboxylic acid piperidin-1-ylamide and 4-chlorophenylzinc iodide, which gave 5.4 mg of the titled compound. MS (ES⁺, M+1)=439.

General Procedure D: Synthesis of Amidines

Imidoyl chloride 11-chloro-dibenzo[b,f] [1,4]thiazepin-8-carboxylic acid piperidin-1-ylamide (5 mg, 0.013 mmol) was mixed with an excess of the appropriate amine in dry toluene. The reaction was shaken for 18 h at 80 degrees C. Concentration of the reaction mixture at reduced pressure gave a crude product, which was purified by column chromatography (ethyl acetate/heptane 1:1 to 3:1).

### Example 35

### 11-piperidinyl-dibenzo[b,f] [1,4]thiazepin-8-carboxylic acid piperidin-1-ylamide

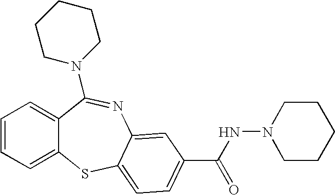

The reaction was performed according to the general procedure D using piperidine, which gave 2.8 mg of the titled compound. MS (ES⁺, M+1)=421.

### Example 36

### 11-(4-morpholinyl)-dibenzo[b,f] [1,4]thiazepin-8-carboxylic acid piperidin-1-ylamide

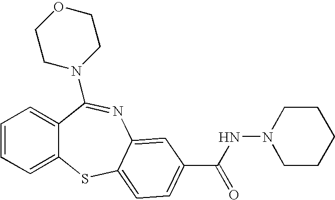

The reaction was performed according to the general procedure D using 7 mg (0.019 mmol) of the imidoyl chloride and morpholine, which gave 5.9 mg of the titled compound. MS (ES⁺, M+1)=423.

### Example 37

### 11-(propylaminyl)-dibenzo[b,f] [1,4]thiazepin-8-carboxylic acid piperidin-1-ylamide

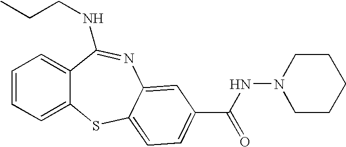

The reaction was performed according to the general procedure D using propyl amine except for applying lower reaction temperature (50 degrees), which gave 2.6 mg of the titled compound. MS (ES⁺, M+1)=395.

### Example 38

### 11-(4-methylpiperazinyl)-dibenzo[b,f] [1,4]thiazepin-8-carboxylic acid piperidin-1-ylamide

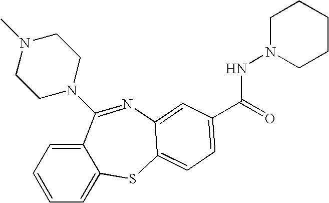

The reaction was performed according to the general procedure D using 10 mg of the imidoyl chloride and methylpiperazine, which gave 7.6 mg of the titled compound. MS (ES⁺, M+1)=436.

### Example 39

### 11-phenylaminyl-dibenzo[b,f] [1,4]thiazepin-8-carboxylic acid piperidin-1-ylamide

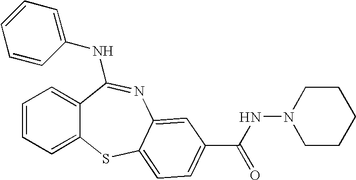

The reaction was performed according to the general procedure D using piperidine, which gave 2.6 mg of the titled compound. MS (ES⁺, M+1)=429.

Synthesis of Carbon Analogs

### Example 40

### 4-(2-Methoxycarbonyl-benzyl)-3-nitro-benzoic acid ethyl ester

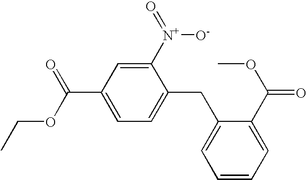

A solution of methyl 2-(bromomethyl)benzoate (261 mg, 1.14 mmol) and tetrakis(triphenylphosphine)palladium(0) (52 mg, 0.045 mmol) in DME (2 mL) under argon was stirred at room temperature for 10 min. 4-Ethoxycarbonyl-2-nitrophenylboronic acid (308 mg, 1.29 mmol) dissolved in DME/EtOH 2:1 (3 mL) was added followed by 2M aq. Na₂CO₃ (2 mL) and stirring was continued for 2 h. The reaction mixture was concentrated in vacuo and purified by column chromatography using EtOAc (0-10%) in heptane as the eluent furnishing 338 mg of 4-(2-Methoxycarbonyl-benzyl)-3-nitro-benzoic acid ethyl ester as a colorless solid (1.13 mmol, 65%).

¹H NMR (400MHz, CDCl₃): 8.58 (d, 2H), 8.06 (dd, 1H), 8.02 (dd, 2H), 7.50 (dt, 1H), 7.38 (dt, 1H), 7.18 (d, 1H), 7.06 (d, 1H), 4.69 (s, 2H), 4.39 (q, 2H), 3.76 (s, 3H), 1.40 (t, 3H).

### Example 41

### 4-(2-Carboxy-benzyl)-3-nitro-benzoic acid

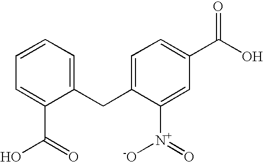

A solution of 4-(2-Methoxycarbonyl-benzyl)-3-nitro-benzoic acid ethyl ester (159 mg, 0.46 mmol) in THF (14 mL) and 1M aq. LiOH (4.6 mL, 4.6 mmol) was stirred at 60° C. for 2 h, then allowed to cool to room temperature. THF was removed at reduced pressure and the resulting aqueous mixture was treated with 2M HCl until the pH was about 1. Filtration provided 93 mg (0.3 mmol, 67%) of 4-(2-Carboxy-benzyl)-3-nitro-benzoic acid as a yellow solid.

¹H NMR (400 MHz, CD₃OD): 8.49 (d, 1H), 8.06 (dd, 1H), 8.02 (dd, 1H), 7.53 (dt, 1H), 7.40 (dt, 1H), 7.26 (d, 1H), 7.12 (d, 1H), 4.69 (s, 2H).

### Example 42

### 3-Amino-4-(2-carboxy-benzyl)-benzoic acid

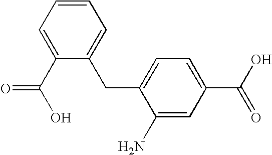

A solution of 4-(2-Carboxy-benzyl)-3-nitro-benzoic acid (79 mg, 0.26 mmol) in MeOH (3 mL) containing PtO₂ (6 mg) and Pd/C (7 mg) was stirred under a hydrogen atmosphere for 2 h at room temperature. Filtration and concentration in vacuo provided 71 mg (0.267 mmol, 100%) of 3-Amino-4-(2-carboxy-benzyl)-benzoic acid as yellow oil.

¹H NMR (400 MHz, CD₃OD): 7.26 (dd, 1H), 7.44-7.38 (m, 2H), 7.32-7.26 (m, 2H), 7.16 (d, 1H), 6.87 (d, 1H), 4.29 (s, 2H).

### Example 43

### 6-Oxo-6,11-dihydro-5H-dibenzo[b,e]azepine-3-carboxylic acid

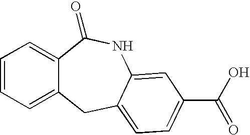

To a stirred solution of 3-Amino-4-(2-carboxy-benzyl)-benzoic acid (70 mg, 0.26 mmol) in THF (3 mL) at room temperature was added carbonyldiimidazole (167 mg, 1.03 mmol) in small portions and stirring was continued. After 4 h, 4M HCl (3 mL) was added followed by water. Filtration and drying provided 51 mg (0.2 mmol, 78%) of 6-Oxo-6,11-dihydro-5H-dibenzo[b,e]azepine-3-carboxylic acid as a colourless solid. The product was further purified by crystallation from 2-propanol.

¹H NMR (400 MHz, DMSO-d₆): 10.58 (s, 1H), 7.70-7.61 (m, 3H), 7.48-7.30 (m, 4H), 3.95 (s, 2H).

### Example 44

### 6-chloro-11H-dibenzo[b,e]azepine-3-carboxylic acid piperidin-1-ylamide

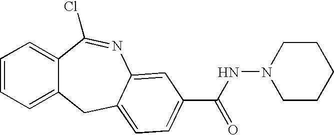

A solution of 6-oxy-5,6-dihydro-1H-dibenzo[b,e]azepine-3-carboxylic acid (45 mg, 0.18 mmol) and phosphorus pentachloride (187 mg, 0.9 mmol) in 2 mL toluene was heated to 90° C. for 6 h. Toluene and excess of phosphorus pentachloride were removed at reduced pressure to give 60 mg of 6-chloro-11H-dibenzo[b,e]azepine-3-carbonyl chloride. 1-Aminopiperidine (0.078 ml, 0.7 mmol) dissolved in CH₂Cl₂ was added to the crude acid chloride dissolved in CH₂Cl₂ at room temperature. EtOAc and H₂O were added to the reaction mixture after 1 h. The H₂O phase was extracted once with EtOAc and the combined organic phases were washed with saturated aqueous NaHCO₃ and brine and dried (Na₂SO₄). Filtration and concentration at reduced pressure of the organic phase followed by purification of the crude product by column chromatography (heptane-EtOAc 1:1) gave 25 mg (40%) of 6-chloro-11H-dibenzo[b,e]azepine-3-carboxylic acid piperidin-1-ylamide. ¹H NMR (400 MHz, CDCl₃) δ 7.81 (d, 2H, J=7.4 Hz), 7.68 (dd, 1H, J=8.0, 1.8 Hz), 7.59 (s, 1H), 7.47 (dt, 1H, J=7.4, 1.2 Hz), 7.33 (t, 1H, J=7.6 Hz), 7.27 (t, 1H, J=7.4 Hz), 3.74 (s, 2H), 2.83 (m, 4H), 1.72 (m, 4H), 1.42 (m, 2H); MS (ES⁺, M+1)=354.

### Example 45

### 6-cyclohexyl-11H-dibenzo[b,e]azepine-3-carboxylic acid piperidin-1-ylamide

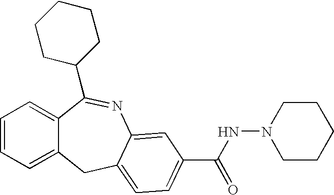

The reaction was performed according to the general procedure for iron-catalyzed alkyl-imidoyl chloride cross coupling using 25 mg of 6-chloro-11H-dibenzo[b,e]azepine-3-carboxylic acid piperidin-1-ylamide and an excess (0.35 ml) of cyclohexylmagnesium chloride (2M). This gave 13.7 mg (49%) of 6-cyclohexyl-11H-dibenzo[b,e]azepine-3-carboxylic acid piperidin-1-ylamide. MS (ES⁺, M+1)=402; UV/MS purity 100/100.

Synthesis of Oxygen Analogs

### Example 46

### 4-(2-Methoxycarbonyl-phenoxy)-3-nitro-benzoic acid ethyl ester

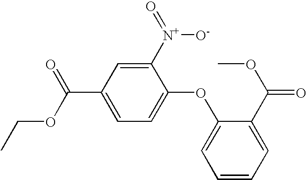

To a stirred solution of ethyl 4-fluoro-3-nitrobenzoate (2.53 g, 11.87 mmol) in DMF (40 mL) containing Cs₂CO₃ (4.26 g, 13.06 mmol) at 100° C. was added drop wise methyl salicylate (1.69 mL, 13.06 mol) dissolved in DMF (40 mL) over 2 h. After 15 min the reaction mixture was allowed to reach room temperature and then diluted with EtOAc (100 mL) and washed with water (2×100 mL). The aqueous layer was extracted with DCM (100 mL). Drying (MgSO₄) of the combined organic layers followed by filtration, concentration in vacuo and purification by CC using EtOAc (0-40%) in heptane provided 3.75 g (10.85 mmol, 91%) of 4-(2-Methoxycarbonyl-phenoxy)-3-nitro-benzoic acid ethyl ester as a yellow solid.

¹H NMR (400 MHz, CDCl₃): 8.60 (d, 1H), 8.04 (dt, 2H), 7.62 (dt, 1H), 7.38 (dt, 1H), 7.19 (dd, 1H), 6.73 (d, 1H), 4.37 (q, 2H), 3.71 (s, 3H), 1.38 (t, 3H).

### Example 47

### 4-(2-Carboxy-phenoxy)-3-nitro-benzoic acid

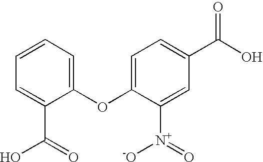

A solution of 4-(2-Methoxycarbonyl-phenoxy)-3-nitro-benzoic acid ethyl ester (3.68 mg, 10.65 mmol) in THF (200 mL) and 1 M aq. LiOH (100 mL, 100 mmol) was stirred at 60° C. for 2 h, then allowed to cool to room temperature. THF was removed at reduced pressure and the resulting aqueous mixture was treated with 2 M HCl until the pH was about 1. Filtration provided 2.75 g (9.08 mmol, 85%) of 4-(2-Carboxy-phenoxy)-3-nitro-benzoic acid as a pale yellow solid.

¹H NMR (400 MHz, CD₃OD): 8.53 (d, 1H), 8.10 (dd, 1H), 8.04 (dd, 1H), 7.69 (dt, 1H), 7.42 (dt, 1H), 7.26 (dd, 1H), 6.82 (d, 1H).

### Example 48

### 3-Amino-4-(2-carboxy-phenoxy)-benzoic acid

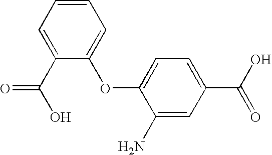

A solution of 4-(2-Carboxy-phenoxy)-3-nitro-benzoic acid (2.75 g, 9.08 mmol) in MeOH (80 mL) containing PtO₂ (59 mg) and Pd/C (211 mg) was stirred for 2 h under a hydrogen atmosphere at room temperature. Filtration and concentration in vacuo provided 2.47 g (9.05 mmol, 100%) of 3-Amino-4-(2-carboxy-phenoxy)-benzoic acid as a pale yellow solid.

¹H NMR (400 MHz, CD₃OD): 7.89 (dd, 1H), 7.54-7.47 (m, 2H), 7.31 (dt, 1H), 7.21 (dt, 1H), 6.97 (d, 1H), 6.68 (d, 1H).

### Example 49

### 11-Oxo-10,11-dihydro-dibenzo[b,f] [1,4]oxazepine-8-carboxylic acid

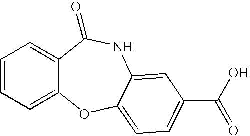

To a stirred solution of 3-Amino-4-(2-carboxy-phenoxy)-benzoic acid (2.44 g, 0.26 mmol) in THF (100 mL) at room temperature was added carbonyldiimidazole (3.7 g, 22.8 mmol) in small portions and stirring was continued. After 4 h, 4 M HCl (100 mL) was added followed by cupious amounts of water. Filtration and drying followed by crystallization (2-propanol) provided 1.017 g (3.99 mmol, 45%) of 11-Oxo-10,11-dihydro-dibenzo[b,f] [1,4]oxazepine-8-carboxylic acid as white crystals.

¹H NMR (400 MHz, DMSO-d₆): 10.61(s, 1H), 7.77-7.74 (m, 2H), 7.67 (dd, 1H), 7.60 (dt, 1H), 7.39 (d, 1H), 7.34 (d, 1H) 7.31 (dt, 1H).

### Example 50

### 11-Chloro-dibenzo[b,f] [1,4]oxazepine-8-carboxylic acid piperidin-1-ylamide

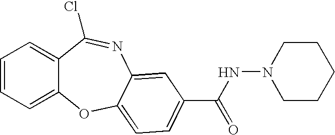

To a stirred solution of 11-Oxo-10,11-dihydro-dibenzo[b,f] [1,4]oxazepine-8-carboxylic acid (476 mg, 1.86 mmol) in toluene (20 mL) and thionyl chloride (20 mL) was added DMF (0.5 mL) and stirring was continued at 80° C. for 19 h. The reaction mixture was concentrated in vacuo and re-dissolved in anhydruos DCM (20 mL) and added to a solution of 1-aminopiperidine (604 μL, 5.59 mmol) dissolved in DCM (20 mL) at 0° C. and stirring was continued for 2 h. The resulting reaction mixture was concentrated in vacuo and purified by CC using EtOAc (0-70%) in heptane affording 353 mg (0.99 mmol, 53%) of 11-Chloro-dibenzo[b,f] [1,4]oxazepine-8-carboxylic acid piperidin-1-ylamide as a pale yellow solid.

¹H NMR (400 MHz, CDCl₃): 7.77-7.72 (m, 2H), 7.63 (s, 1H), 7.53 (dt, 1H), 7.22 (dt, 1H), 7.18 (dd, 1H), 2.92 (br s), 1.76 (br s), 1.43 (br s).

### Example 51

### 11-Cyclohexyl-dibenzo[b,f] [1,4]oxazepine-8-carboxylic acid piperidin-1-ylamide

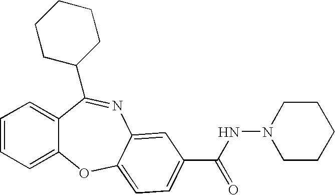

To a flame dried flask loaded with Fe(acac)₃ under argon was added sequentially 11-Chloro-dibenzo[b,f] [1,4]oxazepine-8-carboxylic acid piperidin-1-ylamide (79 mg, 0.22 mmol) dissolved in dry THF, NMP (0.5 mL) and a 2M etheral solution of cyclohexylmagnesium chloride (440 μL, 0.88 mmol) at −78° C. and the reaction micture was allowed to slowly reach ambient temperature. After additionally 2 h sat aq NH₄Cl (5 mL) was added followed by EtOAc (10 mL). After separation of the layers, the aq layer was extracted with EtOAc (2×10 mL). The combined organic layers were dried (MgSO₄), filtered, concentrated in vacuo and purified by CC using EtOAc (0-50%) in heptane as the eluent affording 89 mg (0.22mmol, 100%) of the 11-Cyclohexyl-dibenzo[b,f] [1,4]oxazepine-8-carboxylic acid piperidin-1-ylamide as a grey solid.

¹H NMR (400 MHz, CDCl₃): 7.65 (br s, 1H), 7.63 (br s, 1H), 7.45-7.39 (m, 2H), 7.21 (dt, 1H), 7.15 (dd, 2H), 3.10 (br s), 2.91 (tt), 1.97 (d), 1.85 (br s), 1.74 (d), 1.61 (dd), 1.50 (br s), 1.42-1.29 (m), 1.25 (br s), 0.89-0.85 (m).

### Example 52

### 11-Phenyl-dibenzo[b,f] [1,4]oxazepine-8-carboxylic acid piperidin-1-ylamide

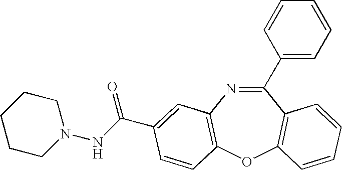

The title compound was synthesised by the same procedure as for preparation of 11-cyclohexyl-dibenzo[b,f] [1,4]oxazepine-8-carboxylic acid piperidin-1-ylamide using 11-chloro-dibenzo[b,f] [1,4]oxazepine-8-carboxylic acid piperidin-1-ylamide (19 mg; 0.05 mmol), phenylmagnesium bromide (3M in diethyl ether; 100 μL; 0.3 mmol), Fe(acac)₃ (3 mg) and NMP (50 μL) in 1 mL dry THF. The titled copound was purified by preparative HPLC. Yield: 5.3 mg. LCMS m/z 398 [M+H]⁺. HPLC t_{R}=7.76 min.

### Example 53

### 11-(4-Fluorophenyl)-dibenzo[b,f] [1,4]oxazepine-8-carboxylic acid piperidin-1-ylamide

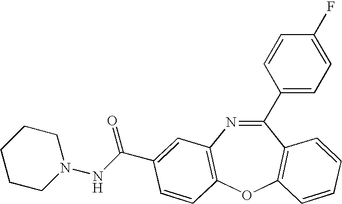

The title compound was synthesised by the same procedure as for preparation of 11-cyclohexyl-dibenzo[b,f] [1,4]oxazepine-8-carboxylic acid piperidin-1-ylamide using 11-chloro-dibenzo[b,f] [1,4]oxazepine-8-carboxylic acid piperidin-1-ylamide (19 mg; 0.05 mmol), 4-fluorophenylmagnesium bromide (2M in diethyl ether; 150 μL; 0.3 mmol), Fe(acac)₃ (3 mg) and NMP (50 μL) in 1 mL dry THF. The titled copound was purified by preparative HPLC. Yield: 3.9 mg. LCMS m/z 416 [M+H]⁺. HPLC t_{R}=7.97 min.

### Example 54

### 11-(4-Chlorophenyl)-dibenzo[b,f] [1,4]oxazepine-8-carboxylic acid piperidin-1-ylamide

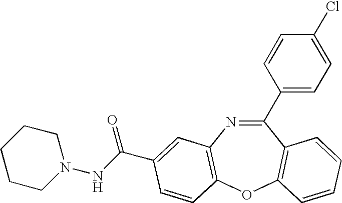

The title compound was synthesised by the same procedure as for preparation of 11-cyclohexyl-dibenzo[b,f] [1,4]oxazepine-8-carboxylic acid piperidin-1-ylamide using 11-chloro-dibenzo[b,f] [1,4]oxazepine-8-carboxylic acid piperidin-1-ylamide (19 mg; 0.05 mmol), 4-chlorophenylmagnesium bromide (1M in diethyl ether; 300 μL; 0.3 mmol), Fe(acac)₃ (3 mg) and NMP (50 μL) in 1 mL dry THF. The titled copound was purified by preparative HPLC. Yield: 2.1 mg. LCMS m/z 432 [M+H]⁺, 434 [M+2+H]⁺. HPLC t_{R}=8.63 min.

### Example 55

### 11-(3-Chlorophenyl)-dibenzo[b,f] [1,4]oxazepine-8-carboxylic acid piperidin-1-ylamide

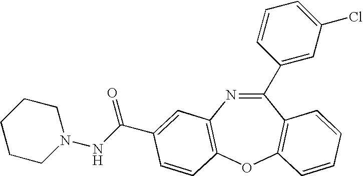

The title compound was synthesised by the same procedure as for preparation of 11-cyclohexyl-dibenzo[b,f] [1,4]oxazepine-8-carboxylic acid piperidin-1-ylamide using 11-chloro-dibenzo[b,f] [1,4]oxazepine-8-carboxylic acid piperidin-1-ylamide (19 mg; 0.05 mmol), 3-chlorophenylzinc iodide (0.5M in THF; 600 μL; 0.3 mmol) and PdCl₂(Ph₃P)₂ (3 mg) in 1 mL dry THF. The titled copound was purified by preparative HPLC. Yield: 8.7 mg. LCMS m/z 432 [M+H]⁺, 434 [M+2+H]⁺. HPLC t_{R}=8.63 min.

Synthesis of 8-bromo analogs:

### Example 56

### 2-(4-Bromo-2-nitrophenylsulfanyl)benzoic acid methyl ester

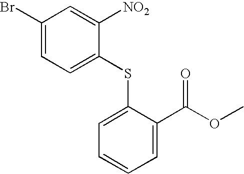

5-Bromo-2-fluoronitrobenzene (1.23 mL; 10.0 mmol) and Cs₂CO₃ (3.58 g; 11.0 mmol) was mixed in 30 mL DMF and heated to 70° C. A solution of methyl 2-mercaptobenzoate (1.5 mL mg; 10.9 mmol) in 30 mL DMF was added dropwise over 15 min. The heating was turned of and the mixture left stirring overnight at room temperature. Water and ethyl acetate was added and the aqueous layer extracted twice with ethyl acetate/heptane. After separation of the phases, the organic phase was washed twice with water, before drying over sodium sulphate, filtration and concentration in vacuo. Purification was done by silica gel column chromatography (0-30% ethyl acetate in heptane) to afford the title compound as a yellow solid (3.61 g; 98%).

¹H NMR (400 MHz, CDCl₃) δ 8.30 (d, 1H, J=2.4), 7.95-7.92 (m, 1H), 7.54-7.45 (m, 4H), 6.86 (d, 1H, J=8.4), 4.82 (s, 3H). HPLC t_{R}=4.97 min.

### Example 57

### 2-(4-Bromo-2-nitrophenylsulfanyl)benzoic acid

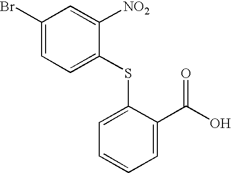

Ester 2-(4-bromo-2-nitrophenylsulfanyl)benzoic acid methyl ester (3.57 g; 9.7 mmol) was dissolved in 120 mL THF and 1M LiOH (aq, 60 mL) added. The solution was heated to 70° C. and stirred at that temperature for 2 hours. The temperature was allowed to cool to room temperature over 3 hours and THF was removed at reduced pressure. The remaining aqueous mixture was extracted once with EtOAc/heptane (1:1, 75 mL). HCl (2M) was then added to the resulting aqueous solution until pH 2. The precipitates were collected by filtration, washed with water and finally dried, which afforded the title compound as a yellow solid (2.82 g; 82%) that was used without further purification.

¹H NMR (400 MHz, CD₃OD) δ 8.59 (d, 1H, J=2.0), 8.02 (dd, 1H, J=2.0, 8.8), 7.94-7.90 (m, 1H), 7.65-7.57 (m, 3H), 7.08 (d, 1H, J=8.8).

### Example 58

### 2-(2-Amino-4-bromophenylsulfanyl)benzoic acid

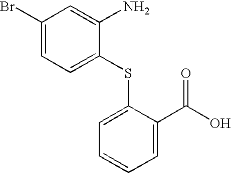

2-(4-Bromo-2-nitrophenylsulfanyl)benzoic acid (1.1 g; 3.1 mmol) was dissolved in 100 mL absolute ethanol and a catalytic amount of palladium on activated carbon was added. The reaction flask was evacuated and equipped with a balloon containing hydrogen. This procedure was repeated twice before the mixture was left stirring overnight at room temperature. The reaction mixture was filtered through a pad of celite and the solvent removed by evaporation to give the crude product (930 mg; 93%) that was used without further purification. LCMS m/z 324 [M+H]⁺, 326 [M+2+H], HPLC t_{R}=10.28 min.

### Example 59

### 8-Bromo-10H-dibenzo[b,f] [1,4]thiazepin-11-one

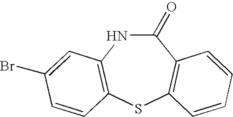

2-(2-Amino-4-bromophenylsulfanyl)benzoic acid (930 mg; 2.87 mmol) was dissolved in 25 mL dry THF and CDI was added (1.4 g mg; 8.61 mmol). The mixture was stirred at room temperature for 2½ days before addition of 4 M aqueous HCl and water. The title compound precipitates and was collected by filtration to afford the desired lactam as colourless crystals (4.45 g; 85%). LCMS m/z 306 [M+H]⁺, 308 [M+2+H], HPLC t_{R}=3.87 min.

### Example 60

### 8-Bromo-11-chloro-dibenzo[b,f] [1,4]thiazepine

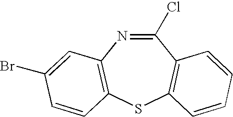

Lactam 8-bromo-10H-dibenzo[b,f] [1,4]thiazepin-11-one (748 mg; 2.44 mmol) was mixed with thionyl chloride (18 mL) in toluene (18 mL). DMF was added (200 μL) and the mixture stirred for 3 hours. After cooling the solvents were removed by evaporation under reduced pressure. Purification was done by silica gel column chromatography (0-20% ethyl acetate in heptane) to afford the imidoyl chloride as a white powder. LCMS m/z 324 [M+H]⁺, 326 [M+2+H]⁺, 328 [M+4+H]⁺, HPLC t_{R}=6.00 min.

The following compounds (Examples 61-66) are examples of compounds synthesised from 8-bromo-11-chloro-dibenzo[b,f] [1,4]thiazepine according to the general procedure for palladium catalysed Negishi couplings and the procedures described by Pandya et al. J. Org. Chem. (2003), 68, 8274-8276 and Sezen and Sames et al., Org Lett. (2003), 5, 3607-3610, which are both incorporated by reference in their entireties.

### Example 61

### 8-Bromo-11-(4-chlorophenyl)-dibenzo[b,f] [1,4]thiazepine

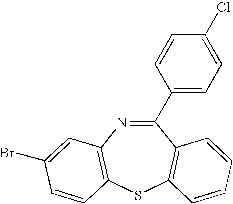

### Example 62

### 11-(4-Chlorophenyl)-dibenzo[b,f] [1,4]thiazepine-8-sulfonic acid butylamide

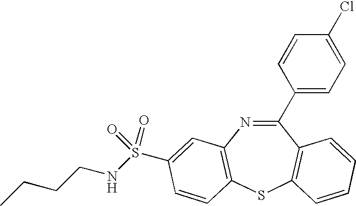

### Example 63

### 11-(4-Chlorophenyl)-dibenzo[b,f] [1,4]thiazepine-8-sulfonic acid piperidin-1-ylamide

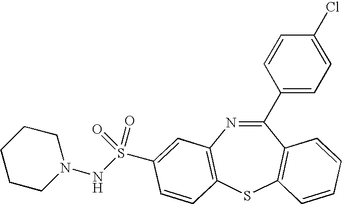

### Example 64

### 11-(4-Chlorophenyl)-8-oxazol-2-yl-dibenzo[b,f] [1,4]thiazepine

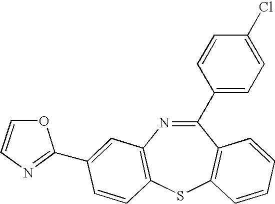

### Example 65

### 11-(4-Chlorophenyl)-8-thiazol-2-yl-dibenzo[b,f] [1,4]thiazepine

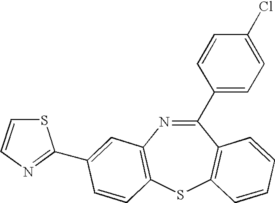

### Example 66

### 11-(4-Chlorophenyl)-8-imidazol-2-yl-dibenzo[b,f] [1,4]thiazepine

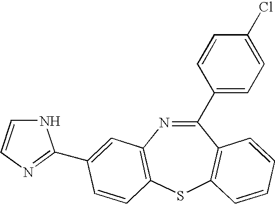 Synthesis of 2-fluoro analogs:

### Example 67

### 4-(4-Fluoro-2-ethoxycarbonylphenylsulfanyl)-3-nitrobenzoic acid ethyl ester

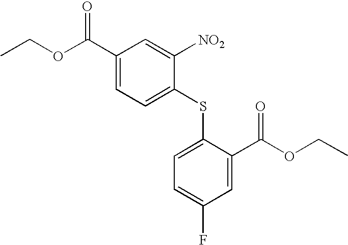

Ethyl 4-fluoro-3-nitrobenzoate (953 mg; 4.47 mmol) and Cs₂CO₃ (1.54 g; 4.72 mmol) were mixed in 20 mL DMF and heated to 80° C. A solution of ethyl 5-fluoro-2-mercaptobenzoate (808 mg; 4.34 mmol) in 20 mL DMF was added dropwise over 15 min. The heating was turned of and the mixture left stirring overnight at room temperature. Water and ethyl acetate was added and the aqueous layer extracted twice with ethyl acetate/heptane. The combined organic phases were washed with water before drying over sodium sulphate, filtration and evaporation of the solvents. Purification was done by silica gel column chromatography (0-10% THF in heptane) to afford the title compound as a yellow oil (1.55 g; 94%).

¹H NMR (400 MHz, CDCl₃) δ 8.84 (d, 1H, J=2.0), 7.96 (dd, 1H, J=1.6, 8.4), 7.67-7.62 (m, 2H), 7.32-7.27 (m, 1H), 6.87 (d, 1H, J=8.4), 4.38 (q, 2H, J=7.2), 4.23 (q, 2H, J=7.2), 1.38 (t, 3H, J=7.2), 1.17 (t, 3H, J=7.2). LCMS m/z 394 [M+H]⁺, HPLC t_{R}=5.43 min.

### Example 68

### 4-(4-Fluoro-2-carboxyphenylsulfanyl)-3-nitrobenzoic acid

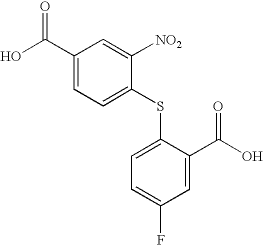

Diester 4-(4-fluoro-2-ethoxycarbonylphenylsulfanyl)-3-nitrobenzoic acid ethyl ester (1.45 g; 3.8 mmol) was dissolved in 100 mL THF and 1M LiOH (aq, 30 mL) added. The solution was heated to 70° C. and stirred at that temperature for 4 hours. The temperature was allowed to cool to room temperature and THF was removed at reduced pressure. The remaining aqueous mixture was extracted once with EtOAc. HCl (2M) was then added to the resulting aqueous solution until pH 2. The precipitates were filtered off, washed with water and finally dried, which afforded the title compound (1.22 g; 99%).

¹H NMR (400 MHz, CD₃OD) δ 8.77 (d, 1H, J=1.6), 8.00 (dd, 1H, J=2.0, 8.4), 7.78-7.73 (m, 2H), 7.45 (dt, 1H, J=3.2, 8.4), 7.01 (d, 1H, J=8.8).

### Example 69

### 3-Amino-4-(4-fluoro-2-carboxyphenylsulfanyl)benzoic acid

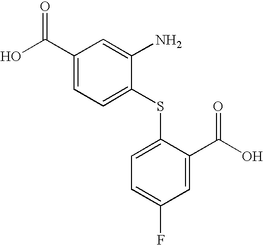

Diacid,4-(4-fluoro-2-carboxyphenylsulfanyl)-3-nitrobenzoic acid (728 mg; 2.16 mmol), was dissolved in 50 mL absolute ethanol and stannous chloride, dihydrate (2.43 g; 10.8 mmol) was added. The temperature was raised to 70° C. and the temperature attained for 15 min. The heating was turned of and the flask slowly allowed to reach room temperature. Water was added and the aqueous phase extracted with ethyl acetate (3 times). The combined organic phases were washed extensively with brine, before drying over sodium sulphate, filtration and removal of the solvent by evaporation. The crude product was obtained as a pale yellow powder (320 mg; 48%) that was used without further purification.

¹H NMR (400 MHz, CD₃OD) δ 7.70 (dd, 1H, J=2.8, 9.2), 7.50 (d, 1H, J=2.0), 7.44 (d, 1H, J=8.0), 7.33-7.29 (m, 1H), 7.12-7.05 (m, 1H), 6.74 (dd, 1H, J=4.8, 9.2).

### Example 70

### 2-Fluoro-11-oxo-10,11-dihydro-dibenzo[b,f] [1,4]thiazepine-8-carboxylic acid

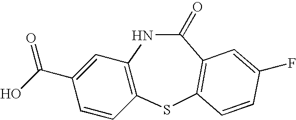

3-Amino-4-(4-fluoro-2-carboxyphenylsulfanyl)benzoic acid (320 mg; 1.04 mmol) was dissolved in 10 mL dry THF and CDI was added (675 mg; 4.17 mmol). The mixture was stirred at room temperature for 2½ days before addition of 4 M aqueous HCl and water. The title compound precipitates and was collected by filtration to afford the desired lactam as colourless crystals (199 mg; 66%).

¹H NMR (400 MHz, DMSO-d₆) δ 7.82-7.75 (m, 1H), 7.54-7.52 (m, 3H), 7.51-7.42 (m, 1H), 7.40-7.29 (m, 1H).

### Example 71

### 11-Chloro-4-fluoro-dibenzo[b,f] [1,4thiazepine-8-carbonyl chloride

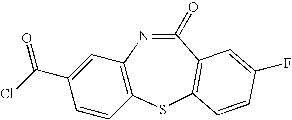

The lactam 2-fluoro-11-oxo-10,11-dihydro-dibenzo[b,f] [1,4]thiazepine-8-carboxylic acid (199 mg; 0.69 mmol) was mixed with thionyl chloride (8 mL) in toluene (8 mL). DMP (100 μL) was added and the mixture stirred at 80° C. overnight. After cooling the solvents were removed by evaporation under reduced pressure to afford the crude, yellow dichloride as a powder that was used immediately without further purification.

### Example 72

### 11-Chloro-2-fluoro-dibenzo[b,f] [1,4]thiazepine-8-carboxylic acid (2-phenylpropyl)-amide

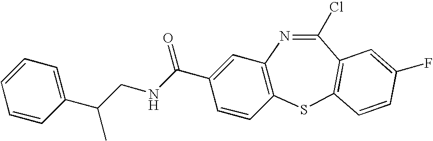

The title compound was synthesized by the general procedure for amide formation using 11-chloro-4-fluoro-dibenzo[b,f] [1,4]thiazepine-8-carbonyl chloride (˜0.35 mmol), 8 mL dry dichloromethane and 2-phenylpropylamine (300 μL; 2.0 mmol). Yield: 87 mg (˜30%).

LCMS m/z 425 [M+H]⁺, 427 [M+2+H]⁺, HPLC t_{R}=5.40 min

### Example 73

### 11-Chloro-2-fluoro-dibenzo[b,f] [1,4]thiazepine-8-carboxylic acid (3-chlorobenzyl)-amide

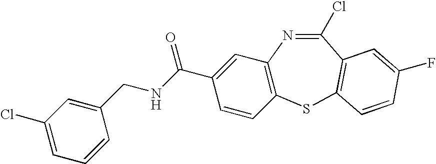

The title compound was synthesized by the general procedure for amide formation using 11-chloro-4-fluoro-dibenzo[b,f] [1,4]thiazepine-8-carbonyl chloride (˜0.35 mmol), 8 mL dry dichloromethane and 3-chlorobenzylamine (252 μL; 2.0 mmol). Yield: 117 mg (˜34%). LCMS m/z 431 [M+H]⁺, 433 [M+2+H]⁺, 436 [M+4+H]⁺. HPLC t_{R}=5.40 min.

### Example 74

### 11-(4-Chlorophenyl)-2-fluoro-dibenzo[b,f] [1,4]thiazepine-8-carboxylic acid (2-phenylpropyl)-amide

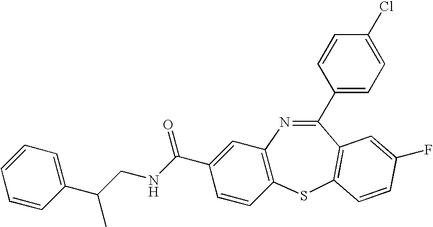

The title compound was synthesized by the general procedure for palladium catalyzed Negishi cross-coupling of amidoimidoyl chlorides and arylzinc halides using 11-chloro-2-fluoro-dibenzo[b,f] [1,4]thiazepine-8-carboxylic acid (2-phenylpropyl)-amide (29 mg; 0.067 mmol) and 4-chlorophenylzinc iodide (0.5 M in THF). The compound was purified by preparative HPLC. Yield: 4.3 mg. LCMS m/z 501 [M+H]⁺, 503 [M+2+H]⁺. HPLC t_{R}=6.68 min.

### Example 75

### 11-(3-Chlorophenyl)-2-fluoro-dibenzo[b,f] [1,4]thiazepine-8-carboxylic acid (2-phenylpropyl)amide

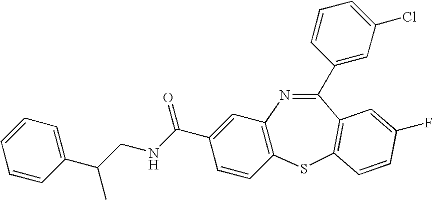

The title compound was synthesised by the general procedure for palladium catalyzed Negishi cross-coupling of amidoimidoyl chlorides and arylzinc halides using 11-chloro-2-fluoro-dibenzo[b,f] [1,4]thiazepine-8-carboxylic acid (2-phenylpropyl)-amide (29 mg; 0.067 mmol) and 3-chlorophenylzinc bromide (0.5 M in THF). The compound was purified by preparative HPLC. Yield: 5.3 mg. LCMS m/z 501 [M+H]⁺, 503 [M+2+H]⁺. HPLC t_{R}=6.65 min.

### Example 76

### 2-Fluoro-11-piperidin-1-yl-dibenzo[b,f] [1,4]thiazepine-8-carboxylic acid (2-phenylpropyl)amide

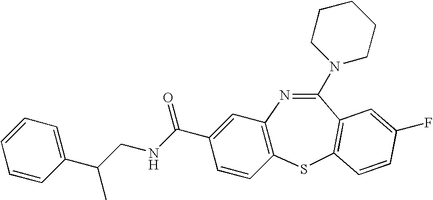

The title compound was synthesised by the general procedure for synthesis of amidines using 11-chloro-2-fluoro-dibenzo[b,f] [1,4]thiazepine-8-carboxylic acid (2-phenylpropyl)amide (29 mg; 0.067 mmol) and piperidine. The title compound was purified by preparative HPLC. Yield: 8.3 mg. LCMS m/z 474 [M+H]⁺. HPLC t_{R}=5.65 min.

### Example 77

### 11-(3-Chlorophenyl)-2-fluoro-dibenzo[b,f] [1,4]thiazepine-8-carboxylic acid (3-chlorobenzyl)-amide

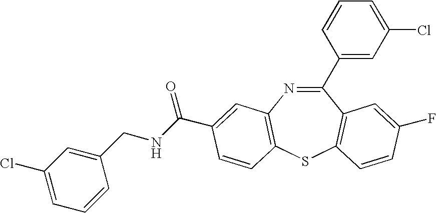

The title compound was synthesised by the general procedure for palladium catalyzed Negishi cross-coupling of amidoimidoyl chlorides and arylzinc halides using 11-chloro-2-fluoro-dibenzo[b,f] [1,4]thiazepine-8-carboxylic acid (3-chlorobenzyl)-amide (22 mg; 0.052 mmol) and 3-chlorophenylzinc bromide (0.5 M in THF). The compound was purified by preparative HPLC. Yield: 2.3 mg. LCMS m/z 507 [M+H]⁺, 509 [M+2+H]⁺. HPLC t_{R}=6.73 min.

### Example 78

### 11-(4-Chlorophenyl)-2-fluoro-dibenzo[b,f] [1,4thiazepine-8-carboxylic acid 3-chlorobenzyl)-amide

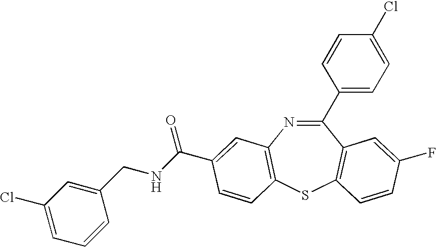

The title compound was synthesised by the general procedure for palladium catalyzed Negishi cross-coupling of amidoimidoyl chlorides and arylzinc halides using 11-chloro-2-fluoro-dibenzo[b,f] [1,4]thiazepine-8-carboxylic acid (3-chlorobenzyl)-amide (22 mg; 0.052 mmol) and 4-chlorophenylzinc iodide (0.5 M in THF). The compound was purified by preparative HPLC. Yield: 5.6 mg. LCMS m/z 507 [M+H]⁺, 509 [M+2+H]⁺. HPLC t_{R}=6.78 min.

### Example 79

### 11-Cyclohexyl-2-fluoro-dibenzo[b,f] [1,4]thiazepine-8-carboxylic acid (3-chlorobenzyl)-amide

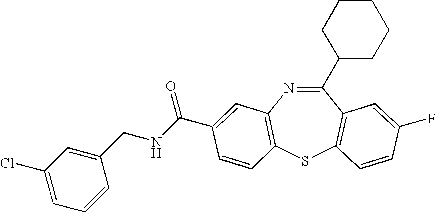

The title compound was synthesised by the general procedure for iron catalyzed cross-couplings using 11-chloro-2-fluoro-dibenzo[b,f] [1,4]thiazepine-8-carboxylic acid (3-chlorobenzyl)-amide (22 mg; 0.052 mmol) and cyclohexylmagnesium chloride (2 M in diethyl ether). The compound was purified by preparative HPLC. Yield: 5.7 mg. LCMS m/z 479 [M+H]⁺, 481 [M+2+H]⁺. HPLC t_{R}=7.37 min.

### Example 80

### 2-Fluoro-11-piperidin-1-yl-dibenzo[b,f] [1,4]thiazepine-8-carboxylic acid (3-chlorobenzyl)-amide

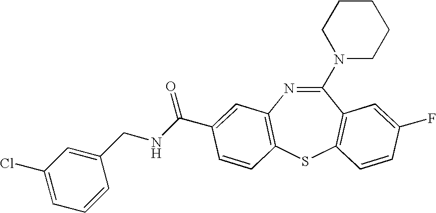

The title compound was synthesised by the general procedure for synthesis of amidines using 11-chloro-2-fluoro-dibenzo[b,f] [1,4]thiazepine-8-carboxylic acid (3-chlorobenzyl)-amide (22 mg; 0.052 mmol) and piperidine. The title compound was purified by preparative HPLC. Yield: 6.5 mg. LCMS m/z 480 [M+H]⁺. HPLC t_{R}=5.77 min.

Synthesis of 3-fluoro and 3-chloro analogs

### Examples 81-104

The synthesis of 3-fluoro and 3-chloro analogs are synthesized using 4-fluoro-2-mercaptobenzoic acid and 3-chloro-2-mercaptobenzoic acid according to the procedures in Marciano et al., Bioorg. Med Chem. Lett. (1997), 7, 1709-1714, which is incorporated by reference in its entirety.

The following compounds are examples several of 3-fluoro and 3-chloro analogs: 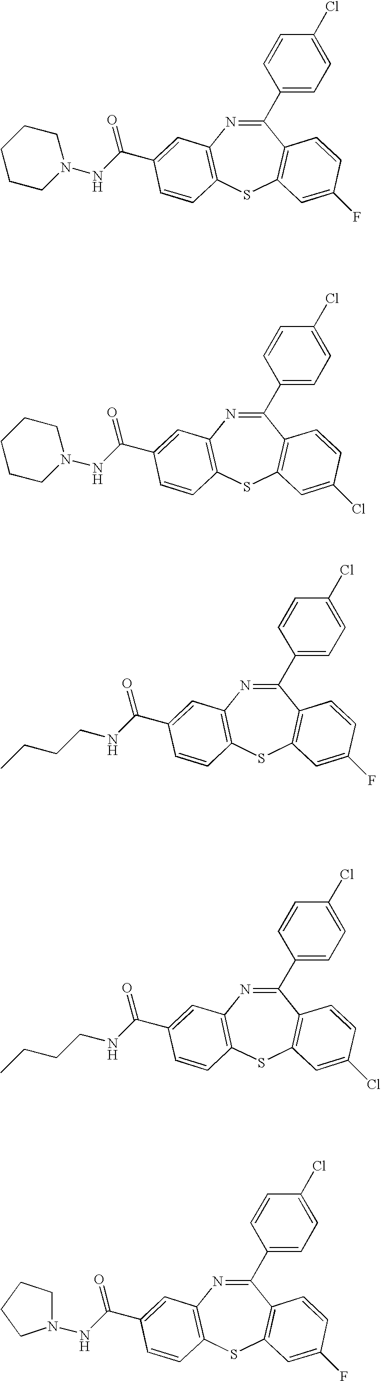 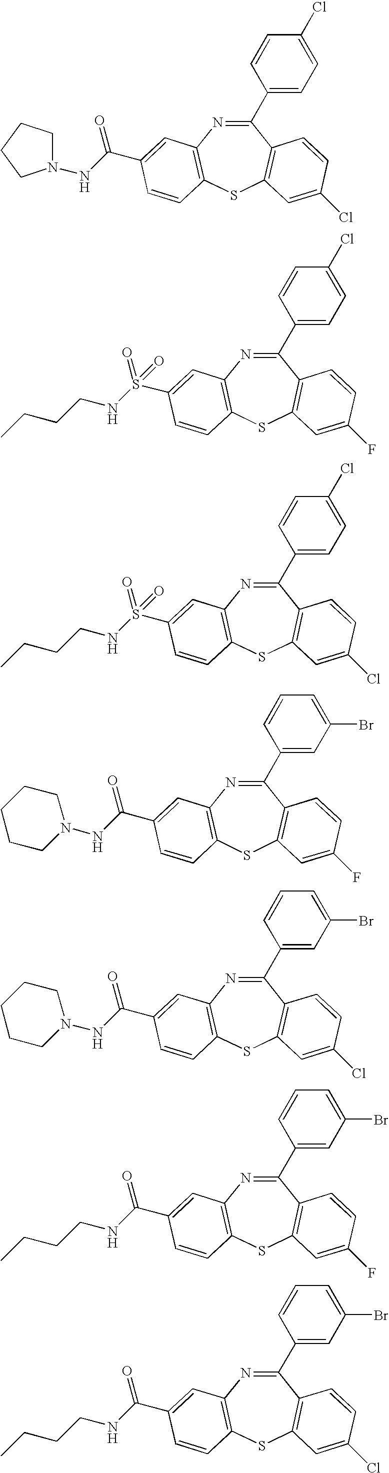 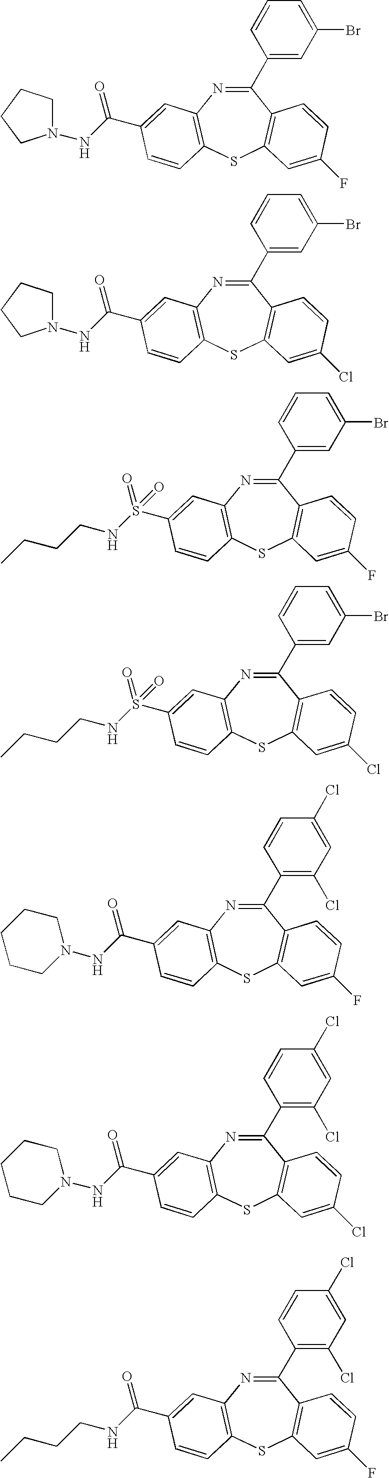 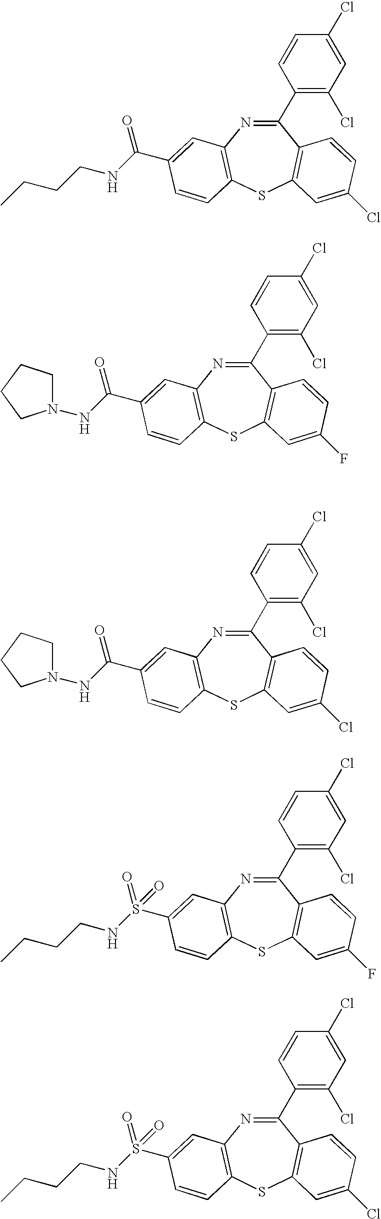
Alternative synthesis of 3-chloro analogs:

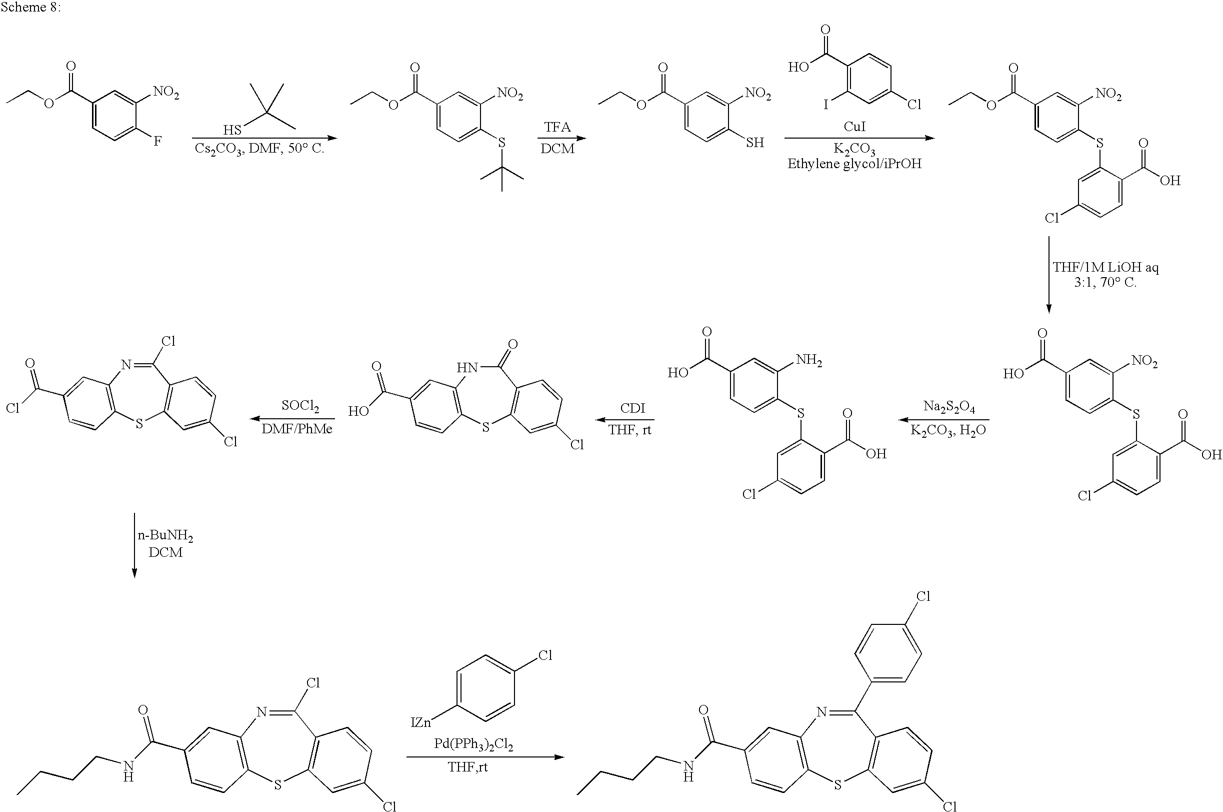

### Example 105

### 4-tert-Butylsulfanyl-3-nitrobenzoic acid ethyl ester

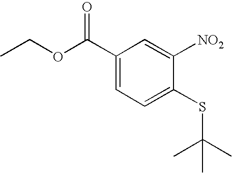

As shown in Scheme 8, ethyl 4-fluoro-3-nitrobenzoate (3.86 g; 18.1 mmol) was dissolved in 90 mL dry DMF together with cesium carbonate (11.8 g; 36.2 mmol). tert-Butylmercaptane (8.15 mL; 72.4 mmol) was added and the mixture was stirred at room temperature for 45 min. Water (50 mL) and ethyl acetate (50 mL) was added and the phases separated. The organic layer was washed with water (2×50 mL) followed by drying over magnesium sulfate. After filtration and evaporation 4.95 g (97%) of a crude yellow oil was isolated that was used without further purification.

R_{f}=0.25 (EtOAc/heptane 30:70). ¹H NMR (CDCl₃, 400 MHz) δ 8.32 (d, 1H, J=2.0, ArH₆), 8.10 (dd, 1H, J=2.0, 8.0, ArH₂), 7.75 (d, 1H, J=8.0, ArH₅), 4.37 (q, 2H, J=7.2, OCH₂), 1.38 (t, 3H, J=7.2, CH₃), 1.35 (s, 9H, tBu).

### Example 106

### 4-Mercapto-3-nitrobenzoic acid ethyl ester

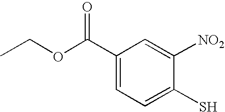

Trifluoroacetic acid (90 mL) was added to a solution of 4-tert-butylsulfanyl-3-nitrobenzoic acid ethyl ester (4.65 g; 16.4 mmol) in 20 mL dichloromethane. The mixture was stirred for 3 days at room temperature before evaporation of the solvent. The residue was partitioned between dichloromethane and 1M aqueous sodium carbonate. After acidification of the aqueous phase using 4M HCl the desired compound was extracted from the aqueous layer with ethyl acetate. The organic layer was dried over sodium sulfate, filtered and evaporated to dryness. The crude compound was used in the next step without purification (1.86 g, 50%).

### Example 107

### 2-(4-(Ethoxycarbonyl)-2-nitrophenlylsulfanyl)-4-chlorobenzoic acid

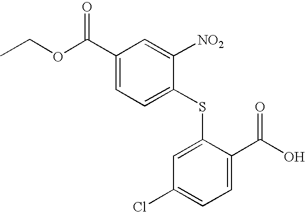

To a mixture of 4-chloro-2-iodobenzoic acid (1.02 g; 3.62 mmol), copper(I)iodide (72.2 mg; 0.17 mmol) and potassium carbonate (947 mg; 6.82 mmol) under argon was added 4-mercapto-3-nitrobenzoic acid ethyl ester (776 mg; 3.41 mmol), ethylene glycol (380 μL; 6.82 mmol) and 10 mL 2-propanol. The mixture was stirred at 80° C. for 1½ h before cooling to room temperature where stirring was attained overnight. Water, 4M HCl and ethyl acetate were added. After separation of the phases the organic phase was washed several times with water, before drying over magnesium sulfate and concentration in vacuo. Purification was done by silica gel column chromatography (0-8% methanol in dichloromethane) to afford the desired compound as yellow crystals (921 mg; 71%).

¹H NMR (CDCl₃, 400 MHz) δ 8.80 (d, 1H, J=2.0, ArH), 8.10-8.02 (m, 2H, ArH), 7.54-7.51 (m, 2H, ArH), 7.07 (d, 1H, J=8.8, ArH), 4.41 (q, 2H, J=7.2, OCH₂), 1.41 (t, 3H, J=7.2, CH₃). LCMS m/z 399 [M+NH₄]⁺, purity (UV/MS) 94/84, t_{R}=7.86 min.

### Example 108

### 4-(3-Chloro-6-carboxyphenylsulfanyl)-3-nitrobenzoic acid

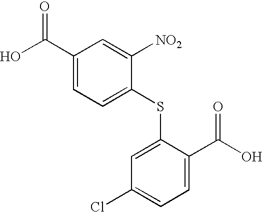

2-(4-(Ethoxycarbonyl)-2-nitrophenylsulfanyl)-4-chlorobenzoic acid (892 mg; 2.34 mmol) was dissolved in a mixture of 1M LiOH (aq, 11 mL) and THF (35 mL). The reaction mixture was stirred at 70° C. for 4 hours. Upon addition of 4M HCl a yellow oil precipitated from the aqueous layer which was extracted with ethyl acetate. The organic layer was dried over magnesium sulfate, filtered and evaporated to dryness affording 1.25 g of which only the majority could be dissolved in ethyl acetate leaving a white solid. After filtration precipitates were accomplished with copious amounts of heptane to afford the title compound as a yellow solid (682 mg; 82%).

LCMS m/z 371 [M+NH₄]⁺, t_{R}=0.67 min.

### Example 109

### 3-Amino-4-(3-chloro-6-carboxyphenylsulfanyl)benzoic acid

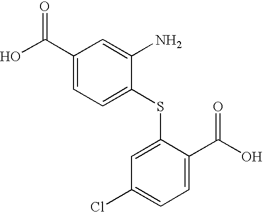

A solution of 4-(3-chloro-6-carboxyphenylsulfanyl)-3-nitrobenzoic acid (680 mg; 1.92 mmol) and potassium carbonate (1.32 g; 9.61 mmol) in 40 mL water was cooled to 0° C. Sodium dithionite (1.67 g; 9.61 mmol) was added portionwise over 5 min. When the shiny yellow colour had disappeared the reaction mixture was allowed to reach room temperature. Drops of 4M HCl were added until precipitates appeared. Ethyl acetate was added (10 mL) and after separation of the layers the organic phase was concentrated in vacuo to afford the title compound as a white crystalline solid. Used immediately without purification.

### Example 110

### 3-Chloro-11-oxo-10,11-dihydro-dibenzo[b,f] [1,4]thiazepine-8-carboxylic acid

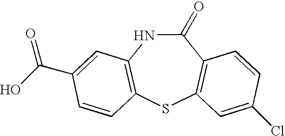

3-Amino-4-(3-chloro-6-carboxyphenylsulfanyl)benzoic acid (˜1.92 mmol) was dissolved in 20 mL dry THF at room temperature. 1,1-Carbonyldiimidazole (1.51 g; 1.52 mmol) was added portionwise and the mixture stirred at room temperature for 2 hours. 4 mL 4M HCl was added ensued by 10 mL of water. The colourless precipitate was collected by filtration to afford the desired compound as a white solid (159 mg; 27% over two steps).

LCMS m/z 306 [M+H]⁺, purity (UV/MS) 98/-, t_{R}=3.47 min.

### Example 111

### 11-Chloro-3-chloro-dibenzo[b,f] [1,4]thiazepine-8-carboxylic acid butyl amide

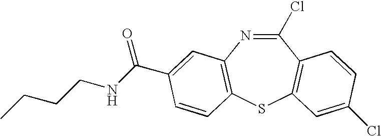

3-Chloro-11-oxo-10,11-dihydro-dibenzo[b,f] [1,4]thiazepine-8-carboxylic acid (38.5 mg; 0.13 mmol), thionyl chloride (2 mL), N,N-dimethylformamide (100 μL) and toluene (2 mL) was heated to 100° C. for 4 hours. The crude mixture was concentrated to dryness to leave the crude acid and imidoyl chloride. The trichloride was redissolved in 5 mL dry dichloromethane and cooled to 0° C. A solution of n-butyl amine (37 μL; 0.38 mmol) in 2 mL dry dichloromethane was added and the mixture stirred for 1 hour. After evaporation of the solvent the residue was purified by silica gel column chromatography (0-30% ethyl acetate in heptane) to afford 27.5 mg of a white solid (58%).

LCMS m/z 379 [M+H]⁺, purity (UV/MS) 100/100,t_{R}=4.70 min.

### Example 112

### 11-(4-Chlorophenyl)-3-chloro-dibenzo[b,f] [1,4]thiazepine-8-carboxylic acid butyl amide

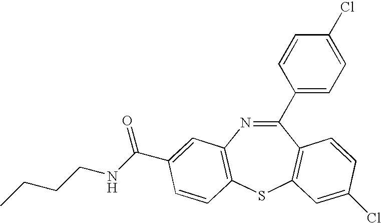

A reaction flask was charged with 11-Chloro-3-chloro-dibenzo[b,f] [1,4]thiazepine-8-carboxylic acid butyl amide (27.5 mg; 0.073 mmol) and bis(triphenylphosphine)palladium(II) chloride (3.3 mg; 0.047 mmol) under argon. 4 mL dry tetrahydrofuran was added and followed by addition of 4-chlorophenylzinc iodide (0.5 M in tetrahydrofuran, 290 μL; 0.145 mmol) at room temperature. The mixture was stirred for ½ hour before evaporation of the solvent. The crude residue was purified by silica gel column chromatography (0-10% ethyl acetate in heptane) to afford the title compound as a yellow oil (25.7 mg; 78%).

¹H NMR (CDCl₃, 400 MHz) δ 7.75-7.71 (m, 2H, ArH), 7.66 (t, 1H, J=1.2, ArH), 7.57 (d, 1H, J=2.0, ArH), 7.51 (d, 2H, J=1.2, ArH), 7.44-7.40 (m, 2H, ArH), 7.30 (dd, 1H, J=2.0, 8.4, ArH), 7.10 (d, 1H, J=8.0, ArH), 6.09 (br m, 1H, NH), 3.47-3.41 (m, 2H, NCH₂), 1.63-1.54 (m, 2H, CH₂), 1.46-1.35 (m, 2H, CH₂), 0.95 (t, 3H, J=7.2, CH₃). ¹³C NMR (CDCl₃, 100 MHz) δ 167.3, 166.6, 148.8, 141.9, 138.3, 137.7, 136.4, 135.3, 133.0, 132.3, 131.5, 131.4, 131.1*, 128.9*, 128.8, 124.6, 124.0, 40.1, 31.9, 20.3, 14.0. *Denotes double intensity. LCMS m/z 454 [M+H]⁺, purity (UV/MS) 100/77, t_{R}=6.88 min.

Synthesis of Sulfoxide and Sulfone Analogs:

The sulfoxides and sulfones described below (Examples 113-121) were synthesized from compounds that have been described previously.

### Example 113

### N-(4-Fluorobenzyl)-11-(4-chlorophenyl)-5-oxo-5H-5λ⁴-dibenzo[b,f] [1,4thiazepine-8-carboxamide

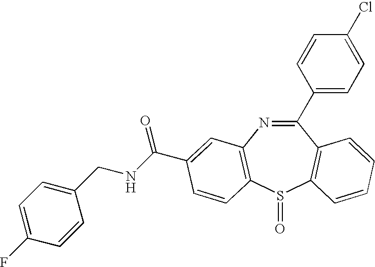

N-(4-Fluorobenzyl)-11-(4-chlorophenyl)-dibenzo[b,f] [1,4]thiazepine-8-carboxamide (182 mg, 0.385 mmol) was suspended in acetic acid (25 mL). Hydrogen peroxide (35% aqueous solution: 1.65 mL) was added dropwise to the suspension at room temperature. After ˜5 hours stirring at room temperature the reaction mixture became clear yellow solution. The stirring was continued overnight at room temperature. The reaction mixture was slowly poured into saturated aqueous sodium bicarbonate (150 mL)—vigorous gas liberation. The neutralized mixture (pH˜7) was extracted with DCM. The organic layer was washed with saturated aqueous sodium bicarbonate, dried over sodium sulphate, filtered and evaporated to dryness. The residue was a mixture of the desired product and the corresponding 5,5-dioxo compound. Purification of the crude mixture by silica gel column chromatography, eluting with a stepwise gradient of 10-30% ethyl acetate in toluene, afforded the desired compound (54 mg, 29%). Rf=0.20 (EtOAc/Toluene 20:80).

¹H NMR (CDCl₃, 300 MHz) δ 7.91-7.70 (m, 7H, Ar—H), 7.50-7.44 (m, 3H, Ar—H), 7.35-7.27 (m, 3H, Ar—H), 7.03 (m, 2H, Ar—H), 6.56 (m, 1H, NH), 4.61 (m, 2H, CH₂PhF). LCMS m/z 489 [M+H]⁺. HPLC t_{R}=5.1 min.

### Example 114

### N-(4-Fluorobenzyl)-11-(4-chlorophenyl)-5,5-dioxo-5H-5λ⁶-dibenzo[b,f] [1,4]thiazepine-8-carboxamide

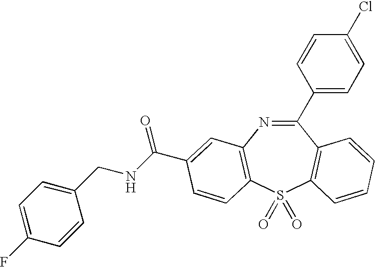

The desired compound was isolated from the crude mixture, which was obtained during the preparation of N-(4-fluorobenzyl)-11-(4-chlorophenyl)-5-oxo-5H-5λ⁴-dibenzo[b,f] [1,4]thiazepine-8-carboxamide. Purification by silica gel column chromatography eluting with a stepwise gradient of 10-30% ethyl acetate in toluene, afforded the desired compound (46 mg, 23%). R_{f}=0.41 (EtOAc/Toluene 20:80).

¹H NMR (CDCl₃, 300 MHz) δ 8.19-8.15 (m, 2H, Ar—H), 7.89-7.66 (m, 6H, Ar—H), 7.47 (m, 3H, Ar—H), 7.33 (m, 2H, Ar—H), 7.06 (m, 2H, Ar—H), 6.50 (m, 1H, NH), 4.63 (m, 2H, CH₂PhF). LCMS m/z 505 [M+H]⁺. HPLC t_{R}=5.1 min.

### Example 115

### N-(3-Chlorobenzyl)-11-(4-fluorophenyl)-5-oxo-5H-5λ⁴-dibenzo[b,f] [1,4]thiazepine-8-carboxamide

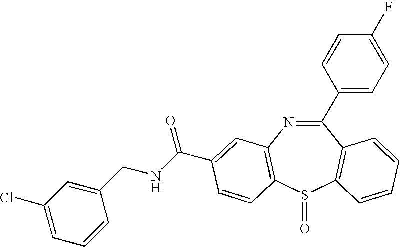

N-(3-Chlorobenzyl)-11-(4-fluorophenyl)-dibenzo[b,f] [1,4]thiazepine-8-carboxamide (25 mg; 0.05 mmol) was dissolved in DCM (3 mL) and 3-chloroperbenzoic acid (26 mg; 0.15 mmol) was added. The mixture was stirred at room temperature for 1 hour. At this point TLC showed full conversion of the starting material and formation of 2 products. The reaction mixture was diluted with DCM and washed three times with saturated aqueous sodium bicarbonate to extract excess 3-chloroperbenzoic acid. The organic phase was dried over sodium sulphate, filtered and evaporated to dryness. Purification was done by silica gel column chromatography eluting with 20-50% ethyl acetate in heptane to give the title compound (9.9 mg).

¹H NMR (acetone-d₆, 400 MHz) δ 8.42 (br s, 1H), 8.01-7.95 (m, 3H), 7.90-7.83 (m, 3H), 7.75 (d, 1H, J=8.0), 7.61 (m, 1H), 7.44-7.40 (m, 2H), 7.34-7.25 (m, 4H), 4.61 (d, 2H, J=6.0). LCMS m/z 489 [M+H]⁺, 491 [M+2+H]⁺. HPLC t_{R}=4.97 min.

### Example 116

### 3-Chlorobenzyl)-11-(4-fluorophenyl)-5,5-dioxo-5H-5λ⁶-dibenzo[b,f] [1,4]thiazepine-8-carboxamide

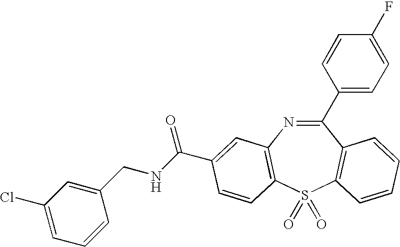

The desired compound was isolated from the crude mixture, which was obtained during the preparation of N-(3-chlorobenzyl)-11-(4-fluorophenyl)-5-oxo-5H-5λ⁴-dibenzo[b,f] [1,4]thiazepine-8-carboxamide. Purification by silica gel column chromatography eluting with a stepwise gradient of 20-50% ethyl acetate in heptane, afforded the desired compound (2.3 mg).

¹H NMR (acetone-d₆, 400 MHz) δ 8.18-8.06 (m, 3H), 7.98-7.85 (m, 5H), 7.65-7.62 (m, 1H), 7.44-7.42 (m, 1H), 7.36-7.26 (m, 5H), 4.66-4.61 (m, 2H), 3.44 (q, 2H, J=7.2), 1.58 (m, 2H, J=7.2), 1.39 (m, 2H, J=7.2), 0.94 (t, 3H, J=7.2). LCMS m/z 505 [M+H]⁺, 507 [M+2+H]⁺. HPLC t_{R}=5.08 min.

### Example 117

### N-butyl-11-(4-chlorophenyl)-5-oxo-5H-5λ⁴-dibenzo[b,f] [1,4]thiazepine-8-carboxamide

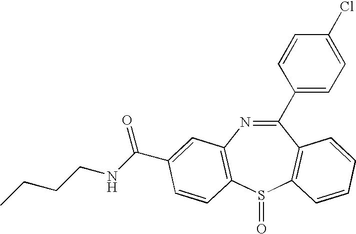

N-Butyl-11-(4-chlorophenyl)-dibenzo[b,f] [1,4]thiazepine-8-carboxamide (86 mg; 0.2 mmol) was dissolved in acetic acid (20 mL) and methanol (15 mL). Hydrogen peroxide (˜35% in water; 1 mL) was added. The reaction mixture was stirred at room temperature for 5 hours before it was neutralized by addition of saturated aqueous sodium bicarbonate. The aqueous solution was extracted with DCM (3×10 mL) and the combined organic phases were washed with water before drying over sodium sulphate, filtration and evaporation of the solvent in vacuo. The resulting residue was purified by silica gel column. chromatography (20-50% ethyl acetate in heptane) followed by preparative TLC on silica eluting 4 times with 5% ethyl acetate in heptane to give the desired compound (20.1 mg; 23%).

¹H NMR (CDCl₃, 400 MHz) δ 7.92-7.89 (m, 1H), 7.80-7.76 (m, 3H), 7.74-7.68 (m, 3H), 7.49-7.42 (m, 3H), 7.26 (dd, 1H, J=0.8, 7.6), 6.21 (m, 1H), 3.44 (q, 2H, J=7.2), 1.58 (m, 2H, J=7.2), 1.39 (m, 2H, J=7.2), 0.94 (t, 3H, J=7.2). LCMS m/z 437 [M+H]⁺, 439 [M+2+H]⁺. HPLC t_{R}=4.83 min.

### Example 118

### N-butyl-11-(4-chlorophenyl)-5,5-dioxo-5H-5λ⁶-dibenzo[b,f] [1,4]thiazepine-8-carboxamide

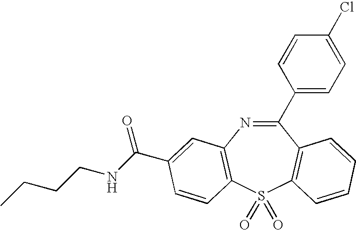

N-Butyl-11-(4-chlorophenyl)-dibenzo[b,f] [1,4]thiazepine-8-carboxamide (70 mg; 0.17 mmol) was dissolved in DCM (10 mL) and 3-chloroperbenzoic acid (225 mg; 1.0 mmol) was added. After 4 hours stirring at room temperature the mixture was diluted with DCM (20 mL) and washed with saturated aqueous sodium hydrogen carbonate (3×15 mL). The organic phase was dried over sodium sulphate, filtered and evaporated to dryness. Purification by preparative TLC eluting twice with 50% ethyl acetate in heptane afforded the title compound (7.9 mg; 10%).

¹H NMR (acetone-d₆, 400 MHz) δ 8.18-8.13 (m, 1H), 8.08 (d, 1H, J=8.0), 8.01 (d, 1H, J=1.6), 7.94-7.86 (m, 5H), 7.67m-7.58 (m, 3H), 3.42 (q, 2H, J=7.4), 1.60 (qn, 2H, J=7.4), 1.40 (m, 2H, J=7.4), 0.93 (t, 3H, J=7.4). LCMS m/z 453 [M+H]⁺, 455 [M+2+H]⁺. HPLC t_{R}=7.93 min.

### Example 119

### 11-(1-Oxy-piperidin-1-yl)-dibenzo[b,f] [1,4]thiazepine-8-carboxylic acid 3-chlorobenzylamide (A) and 5-oxo-11-piperidin-1-yl-5H-5λ⁴-dibenzo[b,f]-[1,4]thiazepine-8-carboxylic acid 3-chlorobenzylamide (B)

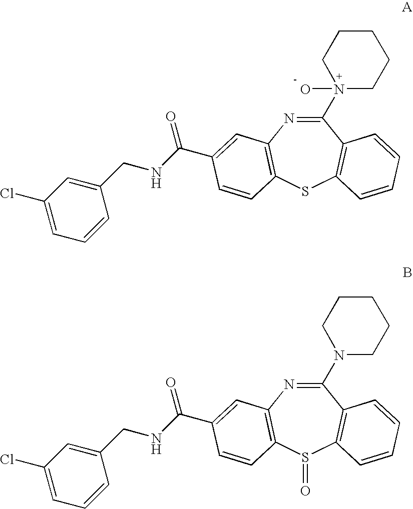

11-Piperidinyl-dibenzo[b,f] [1,4]thiazepine-8-carboxylic acid 3-chlorobenzyl-amide (280 mg; 0.61 mmol) was dissolved in acetic acid (20 mL) and hydrogen peroxide (˜35% in water; 2 mL) added. The mixture was stirred at room temperature for 5 hours. The reaction mixture was neutralized by addition of aqueous saturated NaHCO₃. The aqueous solution was extracted with DCM (3×10 mL) and the combined organic phases were washed with water before drying over sodium sulphate, filtration and evaporation of the solvent in vacuo. Formation of two products was observed by TLC (A: R_{f} 0.06; B: R_{f} 0.25; 1:1 EtOAc/heptane). Both products were isolated by preparative TLC on aluminium oxide eluting twice with 50% ethyl acetate in heptane. Yield: A: 3.0 mg; B: 33 mg as a fine white powder.

A: LCMS m/z 478 [M+H]⁺, 480 [M+2+H]⁺. HPLC t_{R}=4.13 min.

B: ¹H NMR (400 MHz, CD₃Cl) δ 7.83 (dd, 1H, J=1.2, 7.6), 7.63-7.57 (m, 2H), 7.53 (dd, 1H, J=2.0, 8.4), 7.44 (dt, 1H, J=1.2, 7.6), 7.39 (d, 1H, J=1.6), 7.31 (dd, 1H, J=1.2, 7.6), 7.29-7.15 (m, 4H), 6.64 (m, 1H), 4.55 (d, 2H, J=6.0), 3.85-3.30 (br s, 2H), 1.72-1.45 (m, 8H). LCMS m/z 478 [M+H]⁺, 480 [M+2+H]⁺. HPLC t_{R}=4.65 min.

### Example 120

### 5,5-Dioxo-11-piperidin-1-yl-5H-5λ⁴-dibenzo[b,f] [1,4]thiazepine-8-carboxylic acid 3-chlorobenzylamide

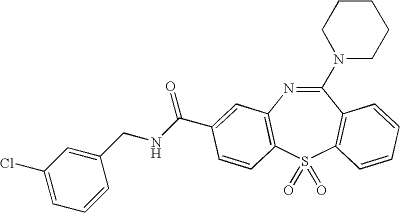

11-Piperidinyl-dibenzo[b,f] [1,4]thiazepine-8-carboxylic acid 3-chlorobenzyl-amide (259 mg; 0.56 mmol) was dissolved in DCM (15 mL) and 3-chloroperbenzoic acid (275 mg; 1.23 mmol) was added. The mixture was stirred at room temperature for 3 hours. The mixture was diluted with 20 mL DCM and washed with saturated aqueous NaHCO₃ (3×15 mL) before drying over sodium sulphate, filtration and removal of the solvent by evaporation under reduced pressure. The crude product was purified by preparative TLC on silica eluting twice with 10% ethyl acetate in heptane to give the title compound (33 mg; 12%).

¹H NMR (400 MHz, CD₃Cl) δ 8.00 (d, 1H, J=8.0), 7.92 (d, 1H, J=8.4), 7.64 (m, 2H), 7.52-7.45 (m, 2H), 7.41 (m, 1H), 7.30-7.17 (m, 4H), 6.46 (m, 1H), 4.60-4.55 (m, 2H), 3.49 (br s, 2H), 1.92-1.44 (m, 8H). LCMS m/z 494 [M+H]⁺, 496 [M+2+H]⁺. HPLC t_{R}=4.93 min.

### Example 121

### 11-Cyclohexyl-5,5-dioxo-5H-5λ⁴-dibenzo[b,f] [1,4]thiazepine-8-carboxylic acid 4-fluorobenzylamide

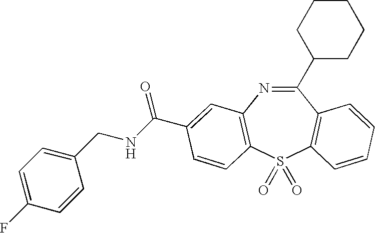

11-Cyclohexyl-dibenzo[b,f]-[1,4]thiazepine-8-carboxylic acid (4-fluorobenzyl)amide (110 mg; 0.25 mmol) was dissolved in DCM (10 mL) and 3-chloroperbenzoic acid (84 mg; 0.37 mmol) was added. The mixture was stirred at room temperature for 2 hours. The mixture was diluted with 10 mL DCM and washed with saturated aqueous NaHCO₃ (3×10 mL) before drying over sodium sulphate, filtration and removal of the solvent by evaporation under reduced pressure. The crude product was purified by preparative TLC on silica eluting 4 times with 5% EtOAc in heptane to give the title compound (2.2 mg). LCMS m/z 477 [M+H]⁺. HPLC t_{R}=5.25 min.

Synthesis of Nitrogen Analogs:

### Example 122

### 8-Chloro-11-(4-fluorophenyl)-5H-dibenzo[b,e] [1,4]diazepine

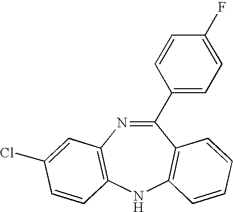

Bis(triphenylphosphine)palladium(II) chloride was added to a solution of 8,11-dichloro-5H-dibenzo[b,e] [1,4]diazepine (100 mg, 0.38 mmol) in anhydrous THF (10 mL) at room temperature under argon atmosphere, followed by addition of 4-fluorophenylzinc bromide (2.28 ml, 1.14 mmol). After 3 hours stirring at room temperature the reaction mixture was partitioned between saturated aqueous ammonium chloride and ethyl acetate. The organic layer was dried over sodium sulphate, filtered and evaporated to dryness. Purification of the residue by silica gel column chromatography, eluting with 30% ethyl acetate in n-heptane, afforded the desired product (88 mg, 72%). R_{f}=0.38 (EtOAc/n-Heptane 30:70). LCMS m/z 323 [M+H]⁺. HPLC t_{R}=5.6 min.

### Example 123

### N-(4-Fluorobenzyl)-11-(4-fluorophenyl)-5H-dibenzo[b,e] [1,4]diazepine-8-carboxamide

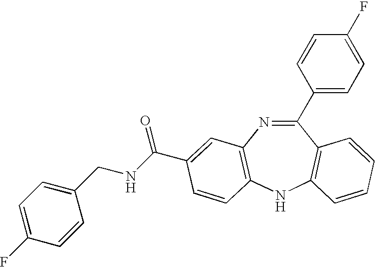

The desired compound was synthesized using a literature procedure in Lagerlund et al., J. Comb. Chem. (2006), 8, 4-6, which is hereby incorporated by reference in its entirety. 8-Chloro-11-(4-fluorophenyl)-5H-dibenzo[b,e] [1,4]diazepine (40 mg, 0.12 mmol) was reacted with 4-fluorobenzylamine (46 mg, 0.37 mmol), molybdenum hexacarbonyl (32 mg, 0.12 mmol), trans-di-(μ-acetato)-bis[o-(di-o-tolylphosphino) benzyl]dipalladium(II) (2.3 mg, 0.025 mmol), tri-tert-butylphosphine tetrafluoroborate (1.7 mg, 0.05 mmol), and 1,8-diazabicyclo[5.4.0]undec-7-ene (56 mg, 0.37 mmol) in anhydrous THF (0.5 mL). The reaction mixture was heated in a sealed flask for 20 minutes at 170° C. under microwave irradiation. The reaction mixture was partitioned between DCM and weak acidic aqueous layer (10 mL water was acidified with 2-3 drops of concentrated HCl). The organic layer was dried over sodium sulphate, filtered and evaporated to dryness. Purification of the residue using a silica gel column chromatography, eluting with a stepwise gradient of 20 to 50% ethyl acetate in n-heptane, afforded the title compound (16 mg, 30%). R_{f}=0.19 (EtOAc/n-Heptane 50:50).

¹H NMR (CDCl₃, 300 MHz) δ 7.75-7.53 (m, 4H, Ar—H), 7.40-7.26 (m, 3H, Ar—H), 7.18-6.92 (m, 6H, Ar—H), 6.87-6.76 (m, 2H, Ar—H), 6.69-6.54 (m, 1H, NH), 5.79-5.56 (m, 1H, NH), 4.59 (m, 2H, CH₂PhF). LCMS m/z 440 [M+H]⁺. HPLC t_{R}=4.6 min.

### Example 124

### N-Butyl-11-(4-fluorophenyl)-5H-dibenzo[b,e][1,4]diazepine-8-carboxamide

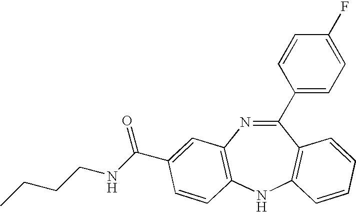

The title compound was synthesized from 8-chloro-11-(4-fluorophenyl)-5H-dibenzo[b,e][1,4]diazepine (25 mg, 0.077 mmol) and n-butylamine (17 mg, 0.23 mmol) using the same procedure as for synthesis of N-(4-fluorobenzyl)-11-(4-fluorophenyl)-5H-dibenzo[b,e][1,4]diazepine-8-carboxamide. R_{f}=0.32 (EtOAc/n-Heptane 50:50). LCMS m/z 388 [M+H]⁺. HPLC t_{R}=4.4 min.

### Example 125

### 11-(4-Fluorophenyl)-N-(1-phenylethyl)-5H-dibenzo[b,e][1,4]diazepine-8-carboxamide

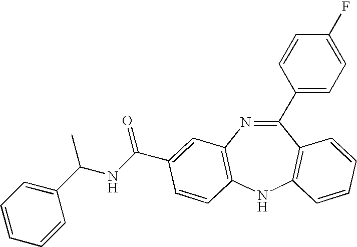

The title compound was synthesized from 8-chloro-11-(4-fluorophenyl)-5H-dibenzo[b,e][1,4]diazepine (25 mg, 0.077 mmol) and DL-1-phenylethyl amine (28 mg, 0.23 mmol) using the same procedure as for synthesis of N-(4-fluorobenzyl)-11-(4-fluorophenyl)-5H-dibenzo[b,e][1,4]diazepine-8-carboxamide. R_{f}=0.33 (EtOAc/n-Heptane 50:50). LCMS m/z 436 [M+H]⁺. HPLC t_{R}=4.7 min.

### Example 126

### 8-Chloro-11-(4-fluorophenyl)-5-methyl-5H-dibenzo[b,e][1,4]diazepine

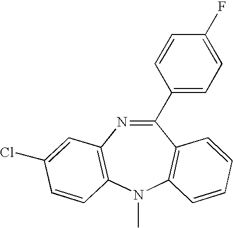

Sodium hydride (60% suspension in an mineral oil: 18 mg, 0.38 mmol) was added to a solution of 8-chloro-11-(4-fluorophenyl)-5H-dibenzo[b,e][1,4]diazepine (60 mg, 0.19 mmol) in dry DMF (2 mL) at room temperature. After 10 minutes shaking at room temperature, the reaction mixture became green and iodomethane (25 μL, 0.38 mmol) was added. The reaction mixture was shaken for 2 hours at 50° C. and then at room temperature overnight. The reaction mixture was partitioned between ethyl acetate and water. The organic layer was washed with 4% aqueous magnesium sulphate, dried over sodium sulphate, filtered and evaporated to dryness. Purification of the residue by silica gel column chromatography, eluting with 10% ethyl acetate in n-heptane, afforded the title compound (40 mg, 60%). R_{f}=0.47 (EtOAc/n-Heptane 30:70). LCMS m/z 337 [M+H]⁺. HPLC t_{R}=6.4 min.

### Example 127

### N-(4-Fluorobenzyl)-11-(4-fluorophenyl)-5-methyl-5H-dibenzo[b,e][1,4]diazepine-8-carboxamide

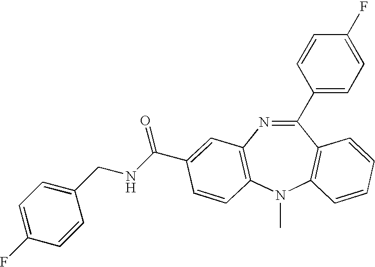

The title compound was synthesized from 8-chloro-11-(4-fluorophenyl)-5-methyl-5H-dibenzo[b,e][1,4]diazepine (20 mg, 0.060 mmol) and 4-fluorobenzyl amine (22 mg, 0.18 mmol) using the same procedure as for synthesis of N-(4-fluorobenzyl)-11-(4-fluorophenyl)-5H-dibenzo[b,e][1,4]diazepine-8-carboxamide. R_{f}=0.32 (EtOAc/n-Heptane 50:50). LCMS m/z 454 [M+H]⁺. HPLC t_{R}=5.0 min.

### Example 128

### 1[8-Chloro-11-(4-fluorophenyl)-dibenzo[b,e][1,4]diazepin-5-yl]ethanone

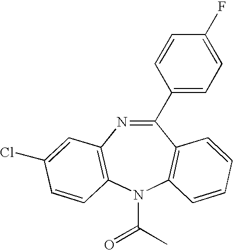

N,N-Dimethyl amine (40 mg, 0.33 mmol) was added to a solution of 8-chloro-11-(4-fluorophenyl)-5H-dibenzo[b,e][1,4]diazepine (108 mg, 0.33 mmol) in dry THF (2 mL) at room temperature, followed by addition of acetyl chloride (70 μL, 0.99 mmol). The reaction mixture was shaken overnight at 60° C., allowed to cool to room temperature and partitioned between ethyl acetate and water. The organic layer was dried over sodium sulphate, filtered and evaporated to dryness. The crude mixture was passed over a short silica gel column using a mixture of ethyl acetate and n-heptane (30:70) as the eluant. The isolated fractions were a mixture of the desired compound and a side product. The fractions were left on standing over the weekend. The desired compound was crystallized in the fractions and it was isolated by filtration (69 mg, 60%). R_{f}=0.20 (EtOAc/n-Heptane 50:50). LCMS m/z 365 [M+H]⁺. HPLC t_{R}=5.0 min.

### Examples 129-146

The following compounds are examples of nitrogen analogs synthesized from 8,11-dichloro-5H-dibenzo[b,e][1,4]diazepine according to the general procedure for palladium catalysed Negishi couplings followed by reductive amination and/or alkylation reactions: 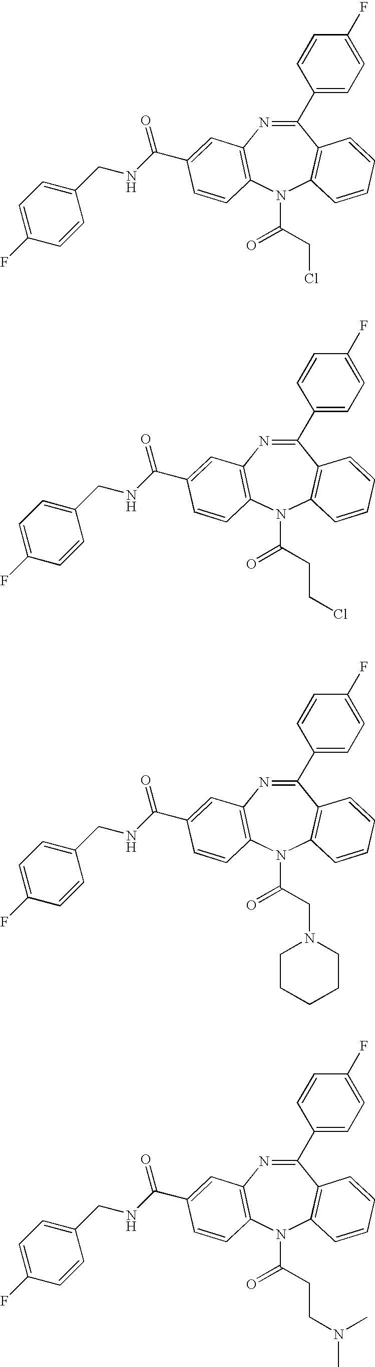 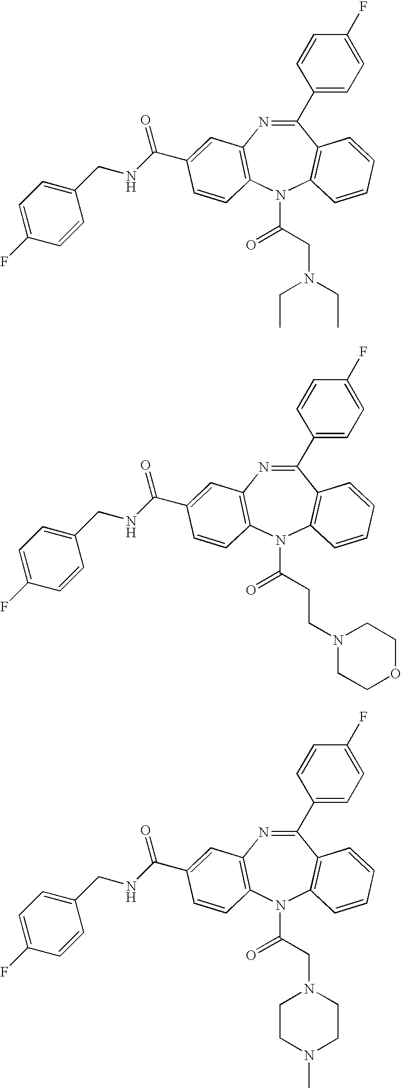 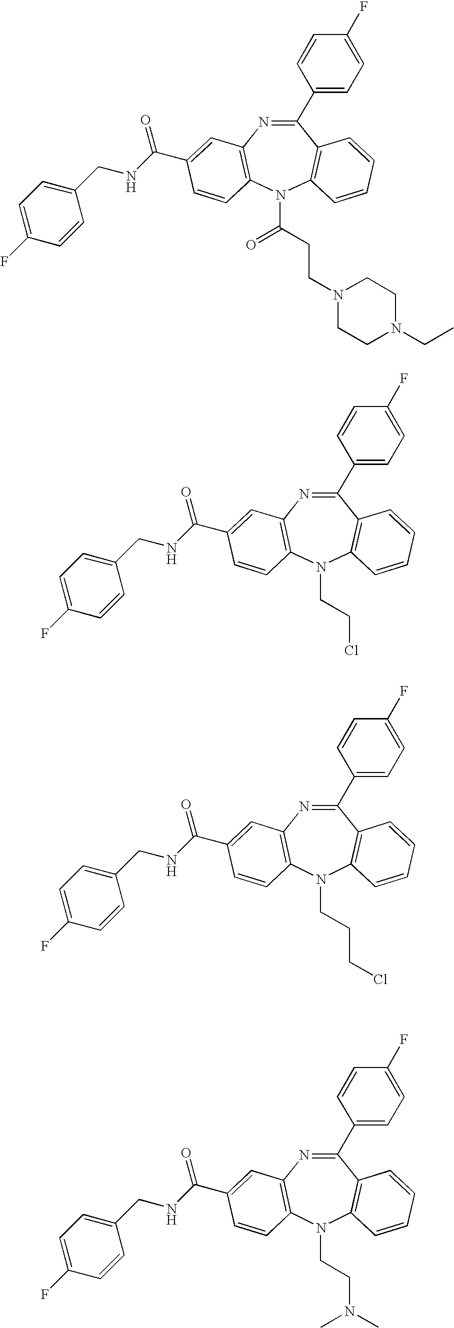 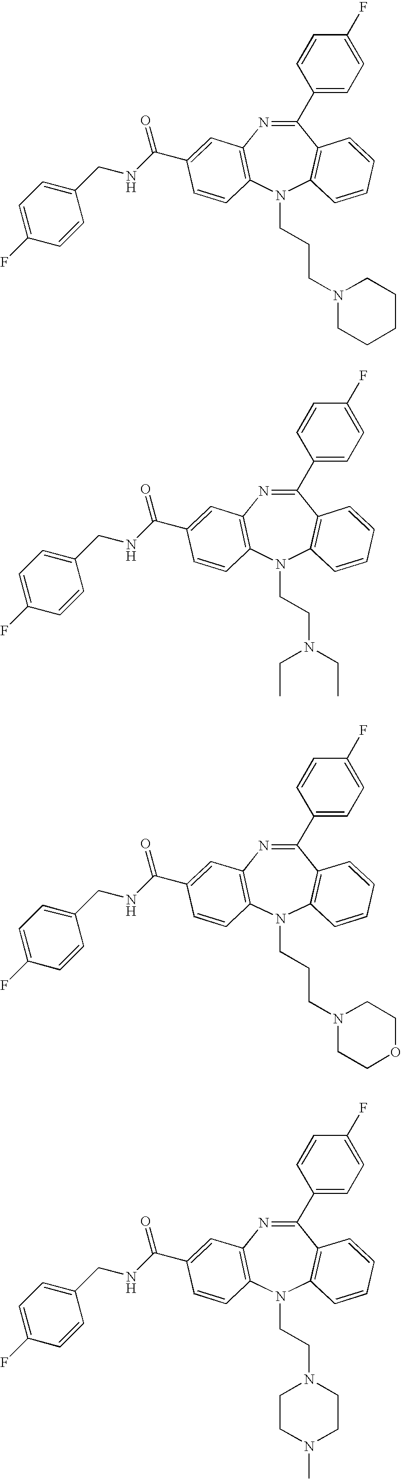 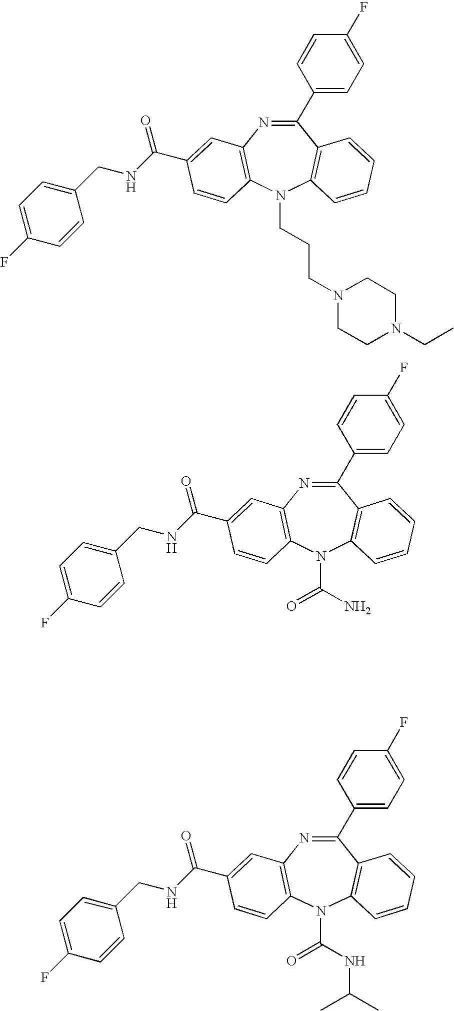
Series A
Library Synthesis; Formation of Amidoimidoyl Chlorides

The amidoimidoyl chlorides (Examples 147-162) were synthesized according to the general procedure for amide formation at 0.5 mmol scale except that the reaction mixture was passed through a pad of acidic alumina oxide and eluted with a mixture of CH₂Cl₂ and EtOAc. The eluents were concentrated at reduced pressure and the obtained crude products were directly used in the next reactions without further purifications or characterization.

### Example 147

### 11-(chloro)-dibenzo[b,f][1,4]thiazepin-8-yl-(piperidin-1-yl)-methanone

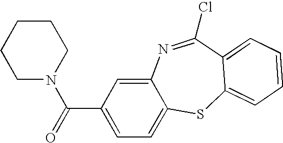

173 mg

### Example 148

### N-benzyl-11-(chloro)-dibenzo[b,f][1,4]thiazepine-8-carboxamide

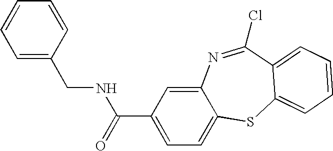

148 mg

### Example 149

### N-(1-phenylethyl)-11-(chloro)-dibenzo[b,f][1,4]thiazepine-8-carboxamide

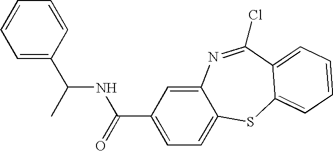

168 mg

### Example 150

### N-(butyl)-11-(chloro)-dibenzo[b,f][1,4]thiazepine-8-carboxamide

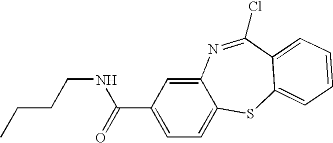

138 mg

### Example 151

### N-(3-phenylpropyl)-11-(chloro)-dibenzo[b,f][1,4]thiazepine-8-carboxamide

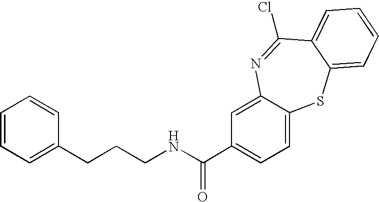

167 mg

### Example 152

### N-(2-phenylethyl)-11-(chloro)-dibenzo[b,f][1,4]thiazepine-8-carboxamide

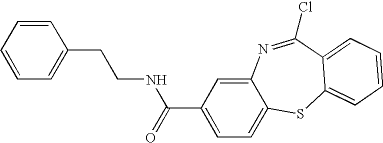

160 mg

### Example 153

### N-(2-chlorobenzyl)-11-(chloro)-dibenzo[b,f][1,4]thiazepine-8-carboxamide

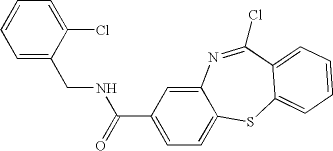

161 mg

### Example 154

### N-(2,4-dichlorobenzyl)-11-(chloro)-dibenzo[b,f][1,4]thiazepine-8-carboxamide

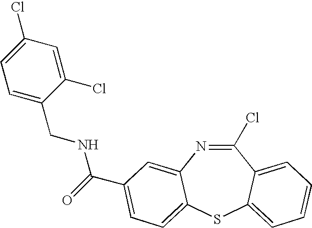

120 mg

### Example 155

### N-(2-(4-chlorophenyl)ethyl)-11-(chloro)-dibenzo[b,f][1,4]thiazepine-8-carboxamide

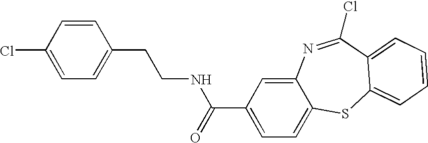

167 mg

### Example 156

### N-(2-(3-chlorophenyl)ethyl)-11-(chloro)-dibenzo[b,f][1,4]thiazepine-8-carboxamide

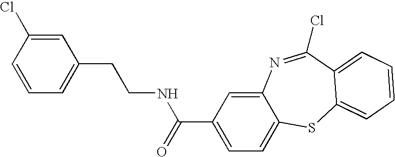

171 mg

### Example 157

### N-(3-chlorobenzyl)-11-(chloro)-dibenzo[b,f][1,4]thiazepine-8-carboxamide

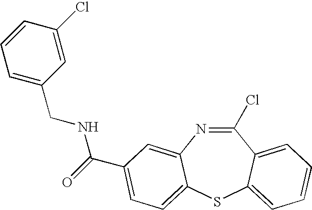

176 mg

### Example 158

### N-(2-bromobenzyl)-11-(chloro)-dibenzo[b,f][1,4]thiazepine-8-carboxamide

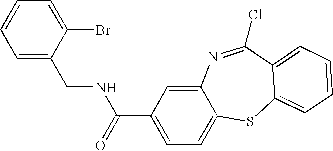

180 mg

### Example 159

### N-(2-phenyl-propyl)-11-(chloro)-dibenzo[b,f][1,4]thiazepine-8-carboxamide

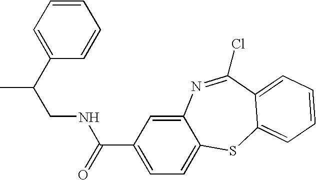

172 mg

### Example 160

### N-((N-ethyl-N-phenyl)aminoethyl)-11-(chloro)-dibenzo[b,f][1,4]thiazepine-8-carboxamide

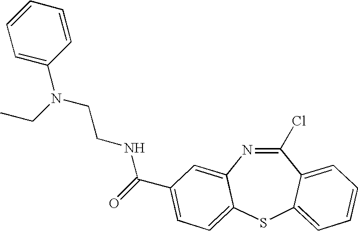

168 mg

### Example 161

### 11-(chloro)-dibenzo[b,f][1,4]thiazepin-8-carboxylic acid morpholin-4-yl amide

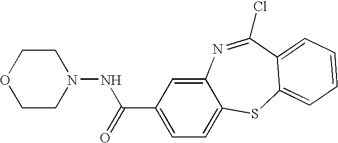

160 mg

### Example 162

### N-(4-fluorobenzyl)-11-(chloro)-dibenzo[b,f][1,4]thiazepine-8-carboxamide

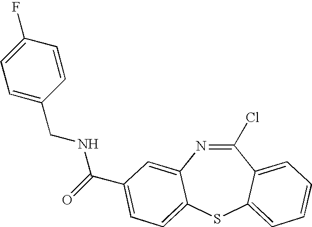

120 mg

Series B

The following compounds were prepared according to the general procedure for the synthesis of amidines starting from the appropriate imidoylchloride (15 mg) and piperidine excess).

### Example 163

### 11-(piperidinyl)-dibenzo[b,f][1,4]thiazepin-8-yl-(piperidin-1-yl)-methanone

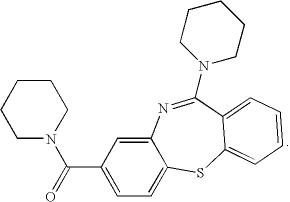

2.8 mg, UV/MS purity 100/97

### Example 164

### N-benzyl-11-(piperidinyl)-dibenzo[b,f][1,4]thiazepine-8-carboxamide

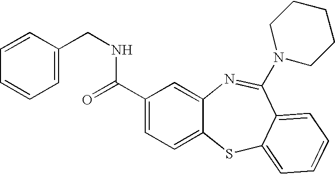

15.9 mg, UV/MS purity 100/91

### Example 165

### N-(1-phenylethyl)-11-(piperidinyl)-dibenzo[b,f][1,4]thiazepine-8-carboxamide

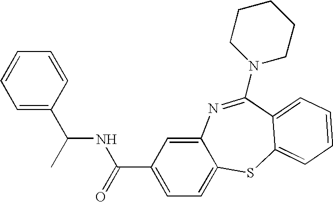

6.2 mg, UV/MS purity 88/54

### Example 166

### N-(butyl)-11-(piperidinyl)-dibenzo[b,f][1,4]thiazepine-8-carboxamide

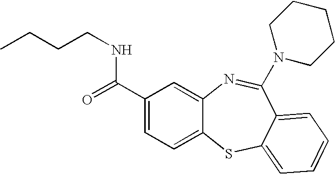

15.1 mg, UV/MS purity 98/80

### Example 167

### N-(3-phenylpropyl)-11-(piperidinyl)-dibenzo[b,f][1,4]thiazepine-8-carboxamide

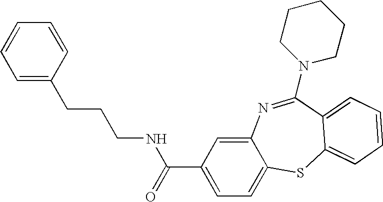

16.7 mg mg, UV/MS purity 100/77

### Example 168

### N-(2-phenylethyl)-11-(piperidinyl)-dibenzo[b,f][1,4]thiazepine-8-carboxamide

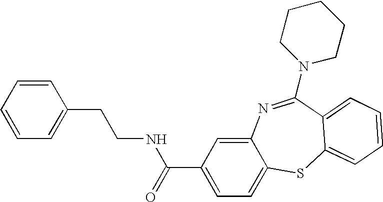

14.5 mg, UV/MS purity 99/76

### Example 169

### N-(2-chlorobenzyl)-11-(piperidinyl)-dibenzo[b,f][1,4]thiazepine-8-carboxamide

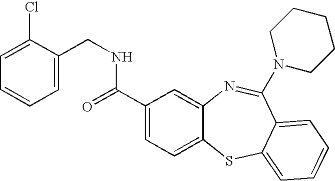

15.2 mg, UV/MS purity 99/73

### Example 170

### N-(2,4-dichlorobenzyl)-11-(piperidinyl)-dibenzo[b,f][1,4]thiazepine-8-carboxamide

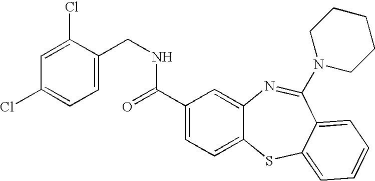

13.2 mg, UV/MS purity 100/73

### Example 171

### N-(2-(4-chlorophenyl)ethyl)-11-(piperidinyl)-dibenzo[b,f][1,4]thiazepine-8-carboxamide

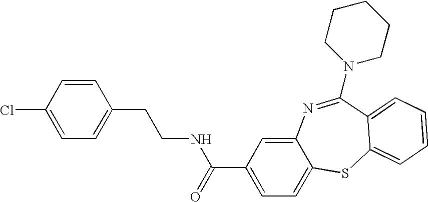

10.7 mg, UV/MS purity 100/79

### Example 172

### N-(2-(3-chlorophenyl)ethyl)-11-(piperidinyl)-dibenzo[b,f][1,4]thiazepine-8-carboxamide

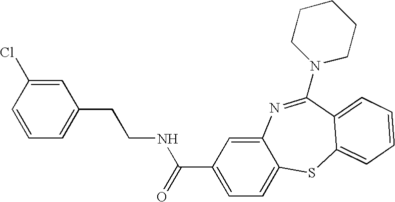

8.4 mg, UV/MS purity 99/67

### Example 173

### N-(3-chlorobenzyl)-11-(piperidinyl)-dibenzo[b,f][1,4]thiazepine-8-carboxamide

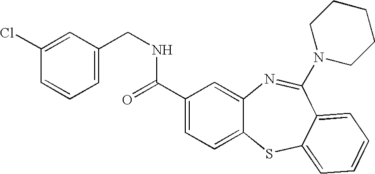

12.9 mg, UV/MS purity 98/72

### Example 174

### N-(2-bromobenzyl)-11-(piperidinyl)-dibenzo[b,f][1,4]thiazepine-8-carboxamide

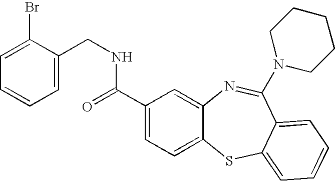

16.2 mg, UV/MS purity 100/76

### Example 175

### N-(2-phenyl-propyl)-11-(piperidinyl)-dibenzo[b,f][1,4]thiazepine-8-carboxamide

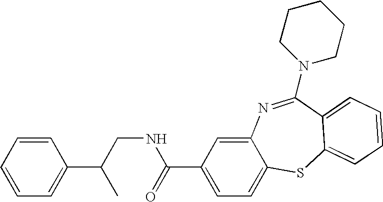

14.2 mg, UV/MS purity 100/72

### Example 176

### N-((N-ethyl-N-phenyl)aminoethyl)-11-(piperidinyl)-dibenzo[b,f][1,4]thiazepine-8-carboxamide

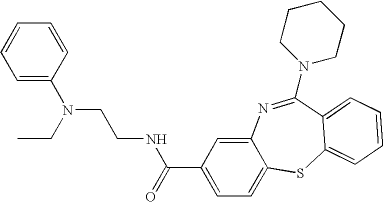

6.0 mg, UV/MS purity 82/60

### Example 177

### 11-(piperidinyl)-dibenzo[b,f][1,4]thiazepin-8-carboxylic acid morpholin-4-yl amide

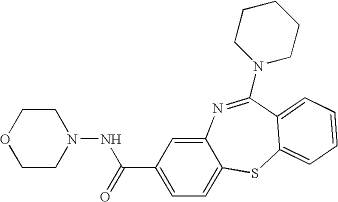

5.9 mg, UV/MS purity 100/78

### Example 178

### N-(4-fluorobenzyl)-11-(piperidinyl)-dibenzo[b,f][1,4]thiazepine-8-carboxamide

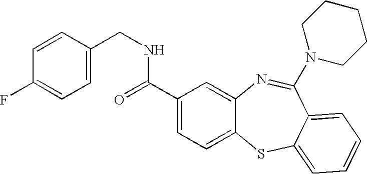

14.7 mg, UV/MS purity 95/53

Series C

The following compounds were prepared according to the general procedure for an iron-catalyzed alkyl-imidoyl chloride cross-coupling starting from the appropriate imidoylchloride (15 mg) and cyclohexylmagnesium chloride (6 eq). When the reactions were completed saturated ammonium chloride (1 ml) and EtOAc (2 ml) were added to the reaction mixtures. The organic phases were passed through a short silica column (eluted with EtOAc). After concentration at reduced pressure, the obtained crude products were purified by preparative HPLC.

### Example 179

### 11-(cyclohexyl)-dibenzo[b,f][1,4]thiazepin-8-yl-(piperidin-1-yl)-methanone

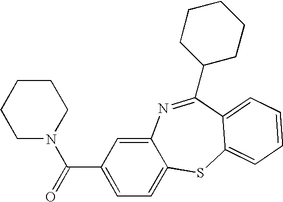

0.6 mg, UV/MS purity 90/90

### Example 180

### N-benzyl-11-(cyclohexyl)-dibenzo[b,f][1,4]thiazepine-8-carboxamide

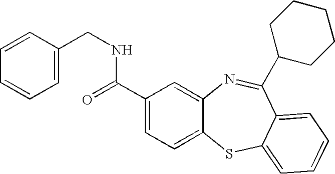

5.1 mg, UV/MS purity 98/83

### Example 181

### N-(1-phenylethyl)-11-(cyclohexyl)-dibenzo[b,f][1,4]thiazepine-8-carboxamide

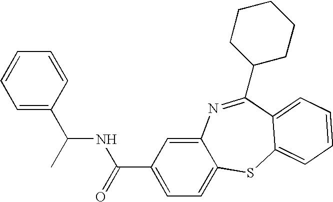

2.2 mg, UV/MS purity 98/87

### Example 182

### N-(butyl)-11-(cyclohexyl)-dibenzo[b,f][1,4]thiazepine-8-carboxamide

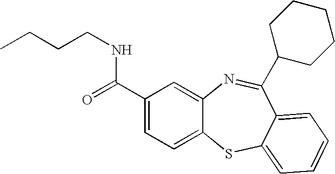

4.6 mg, UV/MS purity 98/91

### Example 183

### N-(2-chlorobenzyl)-11-(cyclohexyl)-dibenzo[b,f][1,4]thiazepine-8-carboxamide

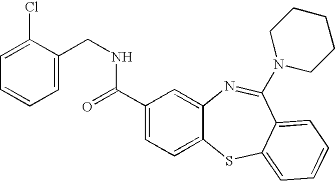

4.5 mg, UV/MS purity 99/85

### Example 184

### N-(2,4-dichlorobenzyl)-11-(cyclohexyl)-dibenzo[b,f][1,4]thiazepine-8-carboxamide

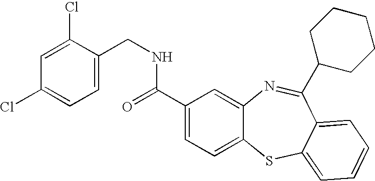

1.8 mg, UV/MS purity 100/82

### Example 185

### N-(2-(4-chlorophenyl)ethyl)-11-(cyclohexyl)-dibenzo[b,f][1,4]thiazepine-8-carboxamide

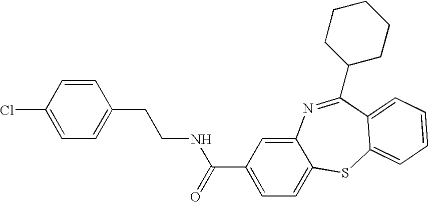

5.9 mg, UV/MS purity 100/87

### Example 186

### N-(2-(3-chlorophenyl)ethyl)-11-(cyclohexyl)-dibenzo[b,f][1,4]thiazepine-8-carboxamide

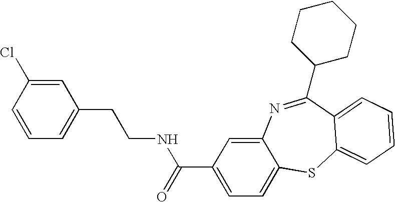

6.6 mg, UV/MS purity 99/90

### Example 187

### N-(3-chlorobenzyl)-11-(cyclohexyl)-dibenzo[b,f][1,4]thiazepine-8-carboxamide

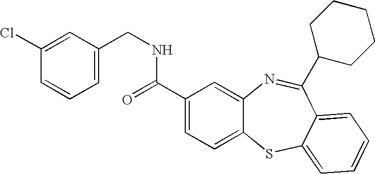

4.8 mg, UV/MS purity 99/87

### Example 188

### N-(2-bromobenzyl)-11-(cyclohexyl)-dibenzo[b,f][1,4]thiazepine-8-carboxamide

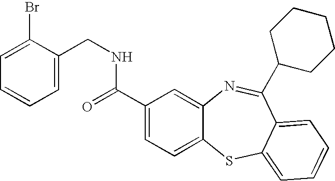

0.8 mg, UV/MS purity 100/83

### Example 189

### N-(2-phenyl-propyl)-11-(cyclohexyl)-dibenzo[b,f][1,4]thiazepine-8-carboxamide

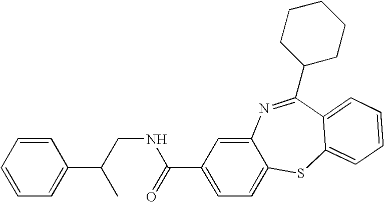

5.3 mg, UV/MS purity 93/83

### Example 190

### N-((N-ethyl-N-phenyl)aminoethyl)-11-(cyclohexyl)-dibenzo[b,f][1,4]thiazepine-8-carboxamide

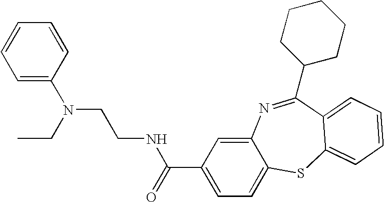

3.2 mg, UV/MS purity 98/79

### Example 191

### 11-(cyclohexyl)-dibenzo[b,f][1,4]thiazepin-8-carboxylic acid morpholin-4-yl amide

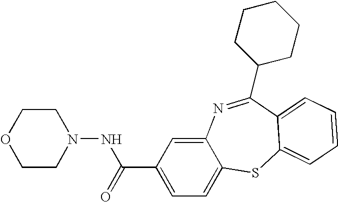

3.8 mg, UV/MS purity 96/75

### Example 192

### N-(4-fluorobenzyl)-11-(cyclohexyl)-dibenzo[b,f][1,4]thiazepine-8-carboxamide

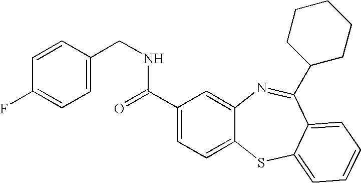

3.6 mg, UV/MS purity 98/74

Series D-H

The following compounds were prepared according to the general procedure for Negishi cross coupling starting from the appropriate imidoylchloride (15 mg) and arylzinc halide (8 eq). Ammonium chloride (0.02 ml) was added to the reaction mixtures, which were then passed through a short column (Na₂SO₄/silica) using EtOAc as eluent. The eluents were concentrated at reduced pressure and the crude products were purified by preparative HPLC or by column chromatography (Heptane-EtOAc 4:1-1:1).

Series D

The arylzinc halide used for Examples 193-205 was 3-chlorophenylzinc iodide.

### Example 193

### 11-(3-chlorophenyl)-dibenzo[b,f][1,4]thiazepin-8-yl-(piperidin-1-yl)-methanone

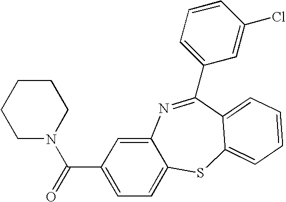

9.9 mg, UV/MS purity 100/80

### Example 194

### N-benzyl-11-(3-chlorophenyl)-dibenzo[b,f][1,4]thiazepine-8-carboxamide

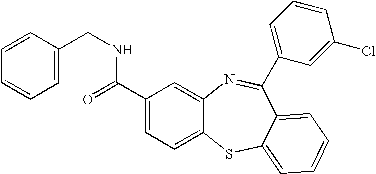

19.2 mg, UV/MS purity 100/60

### Example 195

### N-(1-phenylethyl)-11-(3-chlorophenyl)-dibenzo[b,f][1,4]thiazepine-8-carboxamide

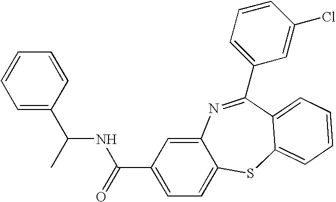

16.7 mg, UV/MS purity 100/85

### Example 196

### N-(butyl)-11-(3-chlorophenyl)-dibenzo[b,f][1,4]thiazepine-8-carboxamide

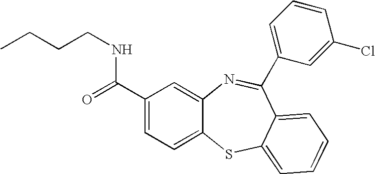

17.3 mg, UV/MS purity 100/79

### Example 197

### N-(3-phenylpropyl)-11-(3-chlorophenyl)-dibenzo[b,f][1,4]thiazepine-8-carboxamide

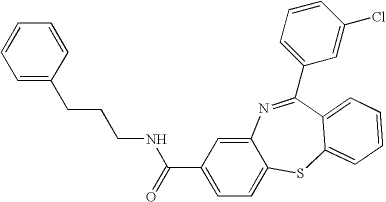

9.0 mg mg, UV/MS purity 95/80

### Example 198

### N-(2-phenylethyl)-11-(3-chlorophenyl)-dibenzo[b,f][1,4]thiazepine-8-carboxamide

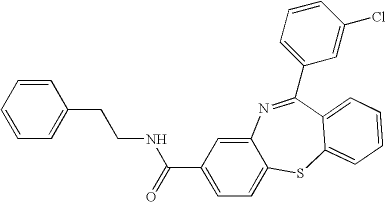

10.9 mg, UV/MS purity 100/80

### Example 199

### N-(2-chlorobenzyl)-11-(3-chlorophenyl)-dibenzo[b,f][1,4]thiazepine-8-carboxamide

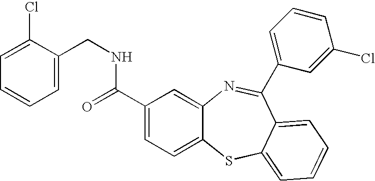

9.7 mg, UV/MS purity 98/80

### Example 200

### N-(2-(4-chlorophenyl)ethyl)-11-(3-chlorophenyl)-dibenzo[b,f][1,4]thiazepine-8-carboxamide

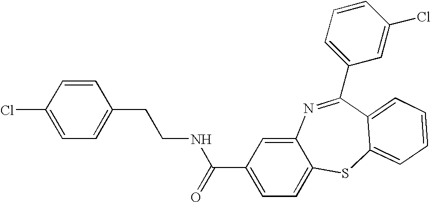

11.2 mg, UV/MS purity 99/76

### Example 201

### N-(3-chlorobenzyl)-11-(3-chlorophenyl)-dibenzo[b,f][1,4]thiazepine-8-carboxamide

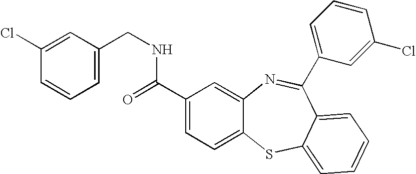

12.2 mg, UV/MS purity 95/72

### Example 202

### N-(2-phenyl-propyl)-11-(3-chlorophenyl)-dibenzo[b,f][1,4]thiazepine-8-carboxamide

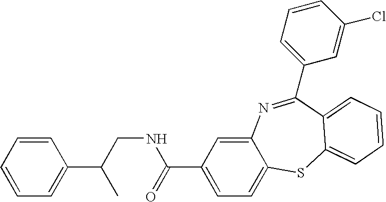

8.8 mg, UV/MS purity 99/59

### Example 203

### N-((N-ethyl-N-phenyl)aminoethyl)-11-(3-chlorophenyl)-dibenzo[b,f][1,4]thiazepine-8-carboxamide

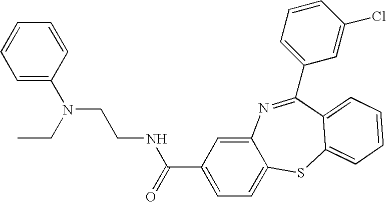

6.3 mg, UV/MS purity 97/80

### Example 204

### 11-(3-chlorophenyl)-dibenzo[b,f][1,4]thiazepin-8-carboxylic acid morpholin-4yl amide

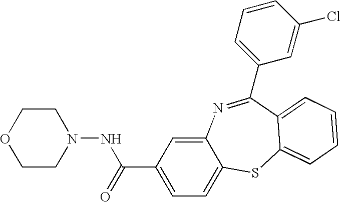

11.8 mg, UV/MS purity 97/56

### Example 205

### N-(4-fluorobenzyl)-11-(3-chlorophenyl)-dibenzo[b,f][1,4]thiazepine-8-carboxamide

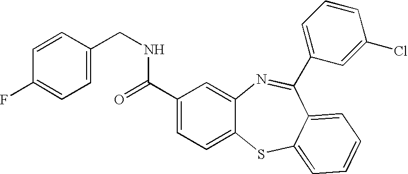

8.1 mg, UV/MS purity 100/55

Series E

The arylzinc halide used for Examples 206-217 was 4-fluorophenylzinc iodide.

### Example 206

### 11-(4-fluorophenyl)-dibenzo[b,f][1,4]thiazepin-8-yl-(piperidin-1-yl-methanone

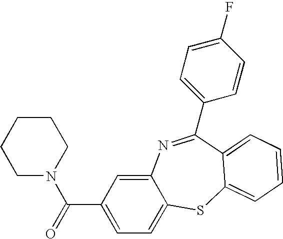

9.9 mg, UV/MS purity 99/62

### Example 207

### N-benzyl-11-(4-fluorophenyl)-dibenzo[b,f][1,4]thiazepine-8-carboxamide

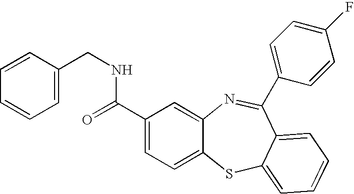

12.2 mg, UV/MS purity 96/41

### Example 208

### N-(1-phenylethyl)-11-(4-fluorophenyl)-dibenzo[b,f][1,4]thiazepine-8-carboxamide

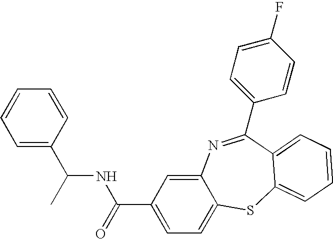

11.4 mg, UV/MS purity 100/91

### Example 209

### N-(butyl)-11-(4-fluorophenyl)-dibenzo[b,f][1,4]thiazepine-8-carboxamide

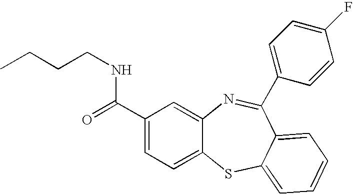

7.5 mg, UV/MS purity 98/93

### Example 210

### N-(2-phenylethyl)-11-(4-fluorophenyl)-dibenzo[b,f][1,4]thiazepine-8-carboxamide

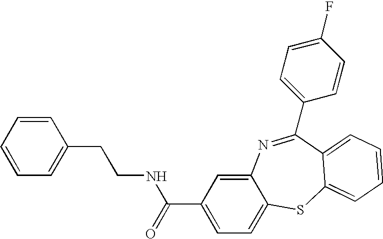

4.6 mg, UV/MS purity 98/62

### Example 211

### N-(2-chlorobenzyl)-11-(4-fluorophenyl)-dibenzo[b,f][1,4]thiazepine-8-carboxamide

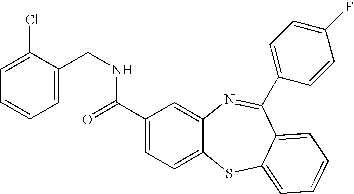

8.4 mg, UV/MS purity 100/52

### Example 212

### N-(2,4-dichlorobenzyl)-11-(4-fluorophenyl)-dibenzo[b,f][1,4]thiazepine-8-carboxamide

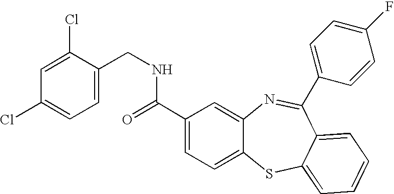

4.0 mg, UV/MS purity 96/36

### Example 213

### N-(2-(4-chlorophenyl)ethyl)-11-(4-fluorophenyl)-dibenzo[b,f][1,4]thiazepine -8-carboxamide

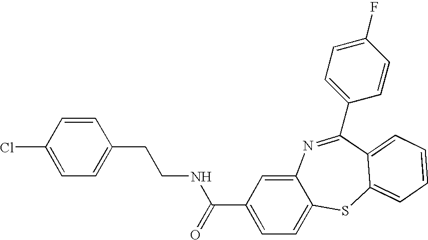

5.6 mg, UV/MS purity 100/65

### Example 214

### N-(2-(3-chlorophenyl)ethyl)-11-(4-fluorophenyl)-dibenzo[b,f][1,4thiazepine-8-carboxamide

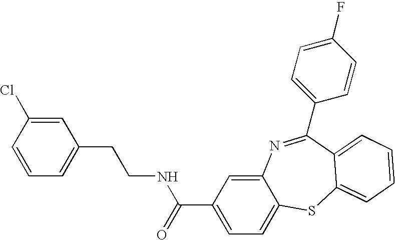

1.4 mg, UV/MS purity 99/56

### Example 215

### N-(3-chlorobenzyl)-11-(4-fluorophenyl)-dibenzo[b,f][1,4]thiazepine-8-carboxamide

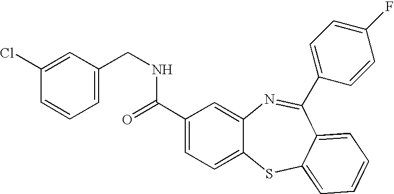

5.4 mg, UV/MS purity 99/50

### Example 216

### N-(2-phenyl-propyl)-11-(4-fluorophenyl)-dibenzo[b,f][1,4]thiazepine-8-carboxamide

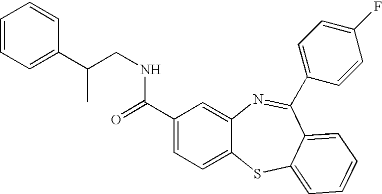

1.9 mg, UV/MS purity 85/44

### Example 217

### N-((N-ethyl-N-phenyl)aminoethyl)-11-(4-fluorophenyl)-dibenzo[b,f][1,4]thiazepine-8-carboxamide

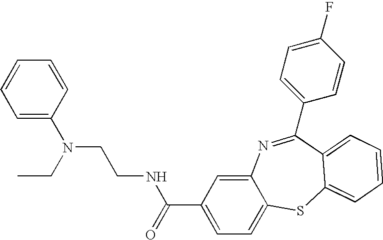

1.3 mg, UV/MS purity 78/45

Series F

The arylzinc halide used for Examples 218-232 was 2-fluorophenylzinc iodide.

### Example 218

### 11-(2-fluorophenyl)-dibenzo[b,f][1,4]thiazepin-8-yl-(piperidin-1-yl)-methanone

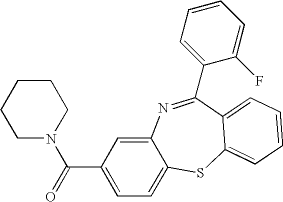

12.5 mg, UV/MS purity 99/67

### Example 219

### N-benzyl-11-(2-fluorophenyl)-dibenzo[b,f][1,4]thiazepine-8-carboxamide

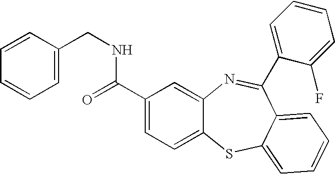

13.7 mg, UV/MS purity 100/100

### Example 220

### N-(1-phenylethyl)-11-(2-fluorophenyl)-dibenzo[b,f][1,4]thiazepine-8-carboxamide

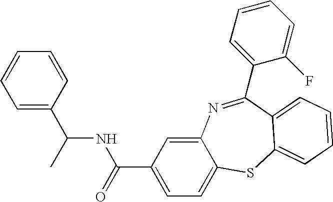

10.1 mg, UV/MS purity 100/96

### Example 221

### N-(butyl)-11-(2-fluorophenyl)-dibenzo[b,f][1,4]thiazepine-8-carboxamide

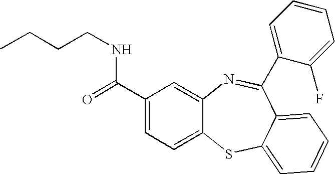

12.3 mg, UV/MS purity 100/94

### Example 222

### N-(3-phenylpropyl)-11-(2-fluorophenyl)-dibenzo[b,f][1,4]thiazepine-8-carboxamide

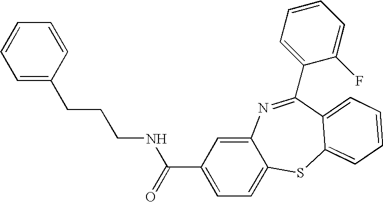

12.3 mg mg, UV/MS purity 100/100

### Example 223

### N-(2-phenylethyl)-11-(2-fluorophenyl)-dibenzo[b,f][1,4]thiazepine-8-carboxamide

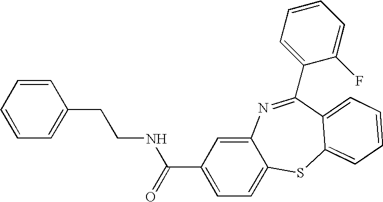

9.3 mg, UV/MS purity 100/100

### Example 224

### N-(2-chlorobenzyl)-11-(2-fluorophenyl)-dibenzo[b,f][1,4]thiazepine-8-carboxamide

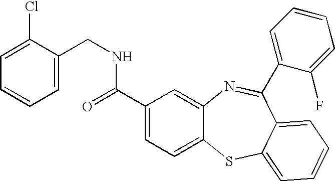

12.7 mg, UV/MS purity 100/89

### Example 225

### N-(2,4-dichlorobenzyl)-11-(2-fluorophenyl)-dibenzo[b,f][1,4]thiazepine-8-carboxamide

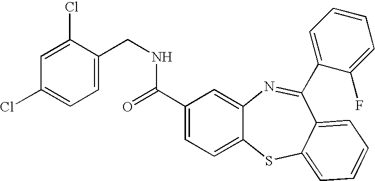

10.6 mg, UV/MS purity 100/84

### Example 226

### N-(2-(4-chlorophenyl)ethyl)-11-(2-fluorophenyl)-dibenzo[b,f][1,4]thiazepine-8-carboxamide

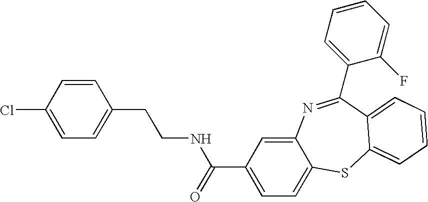

8.4 mg, UV/MS purity 100/92

### Example 227

### N-(2-(3-chlorophenyl)ethyl)-11-(2-fluorophenyl)-dibenzo[b,f][1,4]thiazepine-8-carboxamide

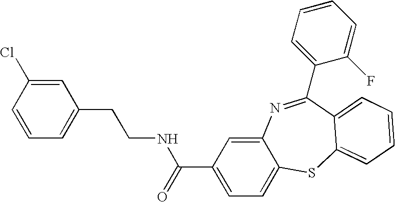

10.4 mg, UV/MS purity 100/91

### Example 228

### N-(3-chlorobenzyl)-11-(2-fluorophenyl)-dibenzo[b,f][1,4]thiazepine-8-carboxamide

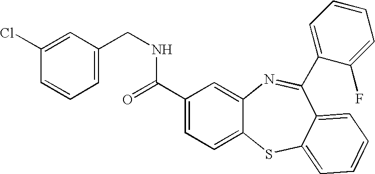

12.5 mg, UV/MS purity 100/95

### Example 229

### N-(2-bromobenzyl)-11-(2-fluorophenyl)-dibenzo[b,f][1,4]thiazepine-8-carboxamide

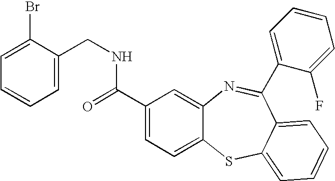

8.3 mg, UV/MS purity 100/96

### Example 230

### N-(2-phenyl-propyl)-11-(2-fluorophenyl)-dibenzo[b,f][1,4]thiazepine-8-carboxamide

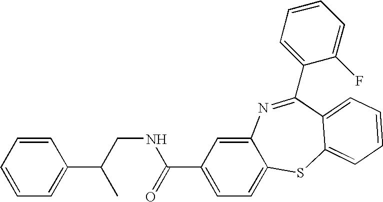

11.2 mg, UV/MS purity 100/90

### Example 231

### N-((N-ethyl-N-phenyl)aminoethyl)-11-(2-fluorophenyl)-dibenzo[b,f][1,4]thiazepine-8-carboxamide

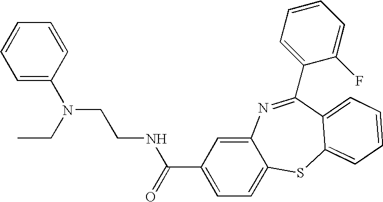

5.7 mg, UV/MS purity 100/91

### Example 232

### N-(4-fluorobenzyl)-11-(2-fluorophenyl)-dibenzo[b,f][1,4]thiazepine-8-carboxamide

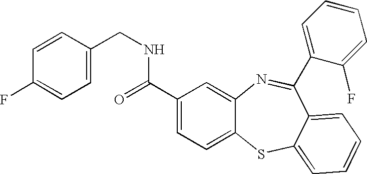

12.4 mg, UV/MS purity 100/91

Series G

The arylzinc halide used for Examples 233-246 was phenylzinc iodide.

### Example 233

### N-benzyl-11-(phenyl)-dibenzo[b,f][1,4]thiazepine-8-carboxamide

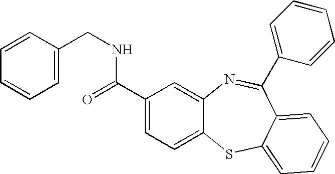

10.2 mg, UV/MS purity 100/57

### Example 234

### N-(1-phenylethyl)-11-(phenyl)-dibenzo[b,f][1,4]thiazepine-8-carboxamide

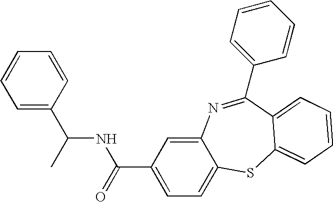

8.2 mg, UV/MS purity 91/61

### Example 235

### N-(butyl)-11-(phenyl)-dibenzo[b,f][1,4]thiazepine-8-carboxamide

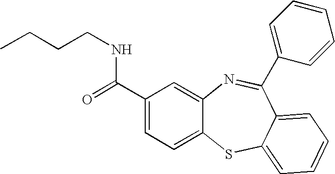

9.4 mg, UV/MS purity 94/62

### Example 236

### N-(3-phenylpropyl)-11-(phenyl)-dibenzo[b,f][1,4]thiazepine-8-carboxamide

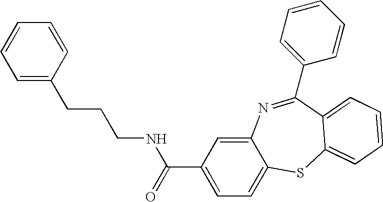

11.4 mg mg, UV/MS purity 100/100

### Example 237

### N-(2-phenylethyl)-11-(phenyl)-dibenzo[b,f][1,4]thiazepine-8-carboxamide

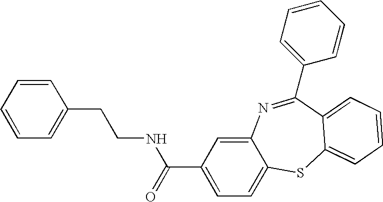

9.0 mg, UV/MS purity 97/85

### Example 238

### N-(2-chlorobenzyl)-11-(phenyl)-dibenzo[b,f][1,4]thiazepine-8-carboxamide

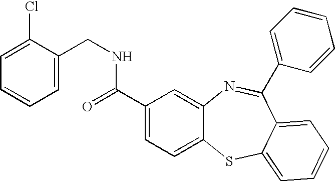

8.8 mg, UV/MS purity 100/100

### Example 239

### N-(2,4-dichlorobenzyl)-11-(phenyl)-dibenzo[b,f][1,4]thiazepine-8-carboxamide

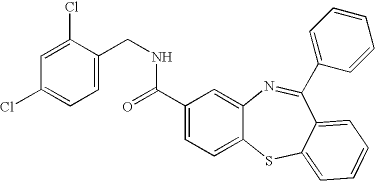

6.1 mg, UV/MS purity 100/87

### Example 240

### N-(2-(4-chlorophenyl)ethyl)-11-(phenyl)-dibenzo[b,f][1,4]thiazepine-8-carboxamide

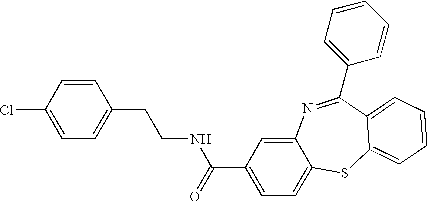

9.3 mg, UV/MS purity 100/90

### Example 241

### N-(2-(3-chlorophenyl)ethyl)-11-(phenyl)-dibenzo[b,f]1,4]thiazepine-8-carboxamide

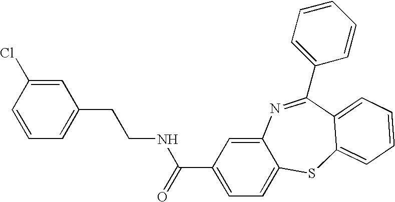

8.9 mg, UV/MS purity 100/80

### Example 242

### N-(3-chlorobenzyl)-11-(phenyl)-dibenzo[b,f][1,4]thiazepine-8-carboxamide

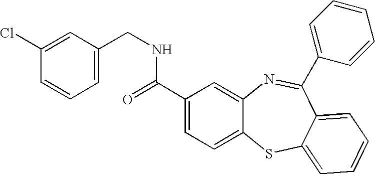

10.1 mg, UV/MS purity 100/100

### Example 243

### N-(2-bromobenzyl)-11-(phenyl)-dibenzo[b,f][1,4]thiazepine-8-carboxamide

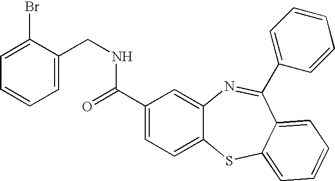

10.2 mg, UV/MS purity 100/89

### Example 244

### N-(2-phenyl-propyl)-11-(phenyl)-dibenzo[b,f][1,4]thiazepine-8-carboxamide

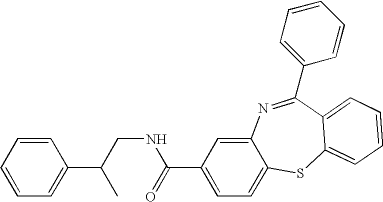

9.5 mg, UV/MS purity 100/87

### Example 245

### N-((N-ethyl-N-phenyl)aminoethyl)-11-(phenyl)-dibenzo[b,f][1,4]thiazepine-8-carboxamide

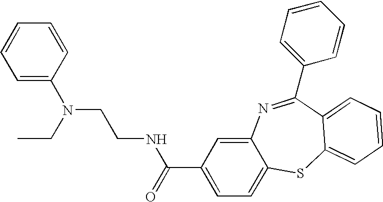

10.0 mg, UV/MS purity 100/91

### Example 246

### N-(4-fluorobenzyl)-11-(phenyl)-dibenzo[b,f][1,4thiazepine-8-carboxamide

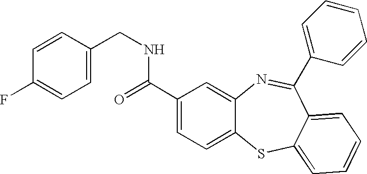

12.8 mg, UV/MS purity 100/93

Series H

The arylzinc halide used for Examples 247-260 was 4-chlorophenylzinc iodide.

### Example 247

### 11-(4-chlorophenyl)-dibenzo[b,f][1,4]thiazepin-8-yl-(piperidin-1-yl)-methanone

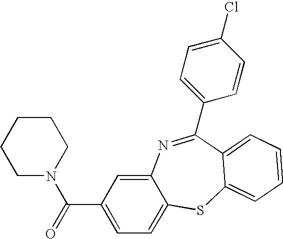

2.2 mg, UV/MS purity 100/100

### Example 248

### N-benzyl-11-(4-chlorophenyl)-dibenzo[b,f][1,4]thiazepine-8-carboxamide

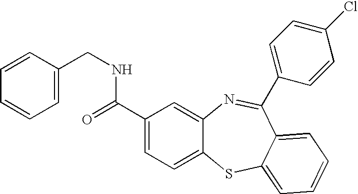

6.3 mg, UV/MS purity 100/100

### Example b 249

### N-(1-phenylethyl)-11-(4-chlorophenyl)-dibenzo[b,f][1,4]thiazepine-8-carboxamide

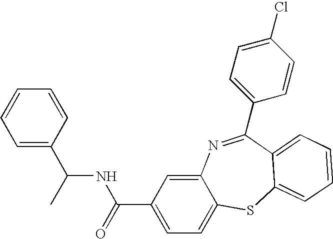

5.7 mg, UV/MS purity 100/83

### Example 250

### N-(butyl)-11-(4-chlorophenyl)-dibenzo[b,f][1,4]thiazepine-8-carboxamide

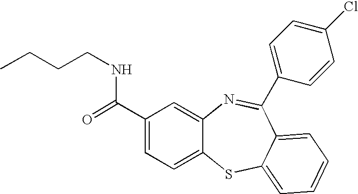

13.7 mg, UV/MS purity 100/100

### Example 251

### N-(3-phenylpropyl)-11-(4-chlorophenyl)-dibenzo[b,f][1,4]thiazepine-8-carboxamide

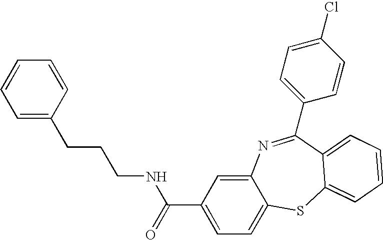

12.5 mg, UV/MS purity 100/100

### Example 252

### N-(2-phenylethyl)-11-(4-chlorophenyl)-dibenzo[b,f][1,4]thiazepine-8-carboxamide

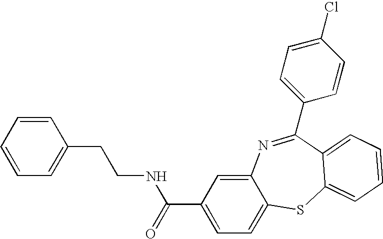

8.7 mg, UV/MS purity 100/100

### Example 253

### N-(2-chlorobenzyl)-11-(4-chlorophenyl)-dibenzo[b,f][1,4]thiazepine-8-carboxamide

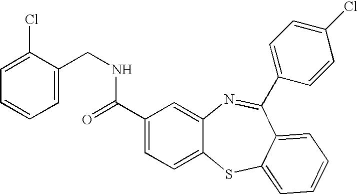

8.4 mg, UV/MS purity 100/100

### Example 254

### N-(2,4-dichlorobenzyl)-11-(4-chlorophenyl)-dibenzo[b,f][1,4]thiazepine-8-carboxamide

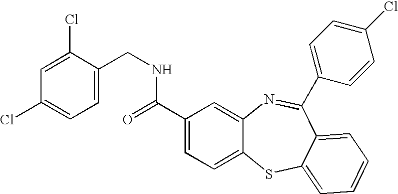

5.4 mg, UV/MS purity 100/73

### Example 255

### N-(2-(4-chlorophenyl)ethyl)-11-(4-chlorophenyl)-dibenzo[b,f][1,4]thiazepine-8-carboxamide

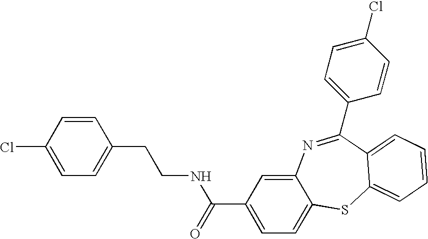

10.2 mg, UV/MS purity 100/80

### Example 256

### N-(2-(3-chlorophenyl)ethyl)-11-(4-chlorophenyl)-dibenzo[b,f][1,4]thiazepine-8-carboxamide

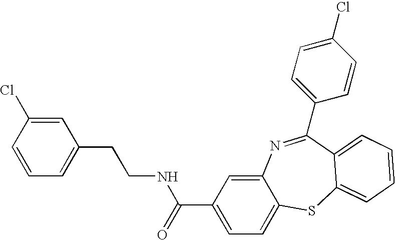

10.0 mg, UV/MS purity 100/100

### Example 257

### N-(3-chlorobenzyl)-11-(4-chlorophenyl)-dibenzo[b,f][1,4]thiazepine-8-carboxamide

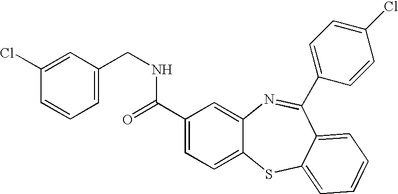

10.0 mg, UV/MS purity 100/100

### Example 258

### N-(2-bromobenzyl)-11-(4-chlorophenyl)-dibenzo[b,f][1,4]thiazepine-8-carboxamide

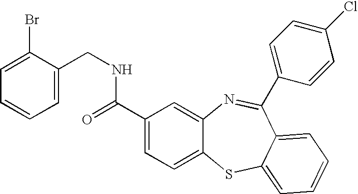

10.2 mg, UV/MS purity 100/67

### Example 259

### N-(2-phenyl-propyl)-11-(4-chlorophenyl)-dibenzo[b,f][1,4]thiazepine-8-carboxamide

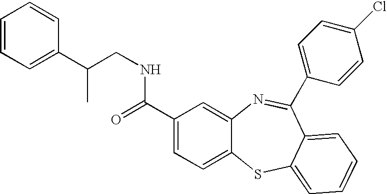

11.9 mg, UV/MS purity 100/100

### Example 260

### N-((N-ethyl-N-phenyl)aminoethyl)-11-(4-chlorophenyl-dibenzo[b,f][1,4]thiazepine-8-carboxamide

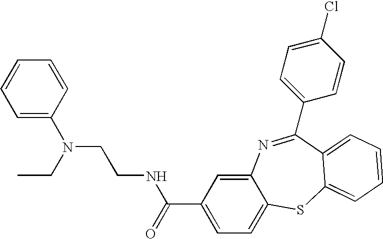

12.4 mg, UV/MS purity 100/88

### Example 261

### N-(4-fluorobenzyl)-11-(4-chlorophenyl)-dibenzo[b,f][1,4]thiazepine-8-carboxamide

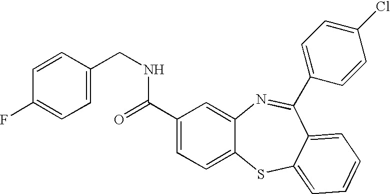

12.8 mg, UV/MS purity 100/100

Series I

The amidoimidoyl chlorides (Examples 262-271) were synthesized according to the general procedure for amide formation using 11-Chloro-dibenzo[b,f][1,4]thiazepine-8-carbonyl chloride (300 mg, 1 mmol) and the proper amine (3 mmol) except that the reaction mixture was passed through a pad of acidic alumina oxide and eluted with a mixture of CH₂Cl₂ and EtOAc. The eluents were concentrated at reduced pressure and the obtained crude products were directly used in the next reactions without further purifications or characterization.

### Examples 262-271

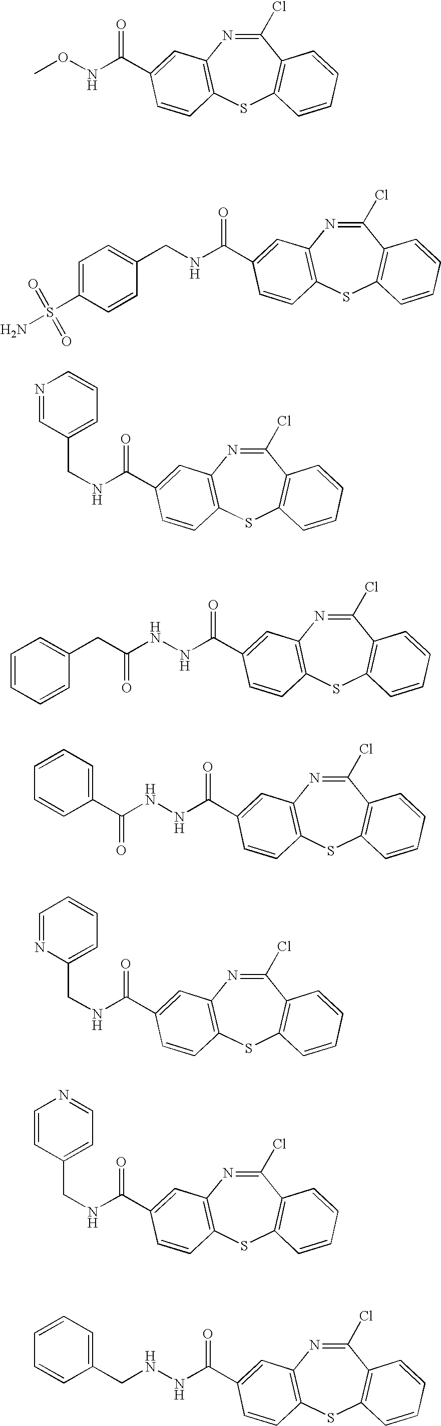 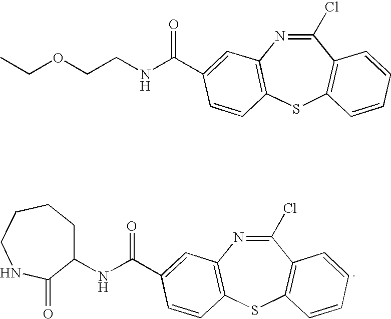 Series J

### Examples 272-301

Examples 272-301 are prepared according to the general procedure for the synthesis of amidines starting from 15 mg of the appropriate amidoimidoyl chloride (represented by titled compounds in Examples 262-271) and the appropriate amine (excess), except that purification is performed by eluting (EtOAc) the products through a pad of silica. The eluents are concentrated at reduced pressure to give the crude products, which are purified by preparative HPLC/MS. Yield is determined by weighing and purity by analytical LC/MS). 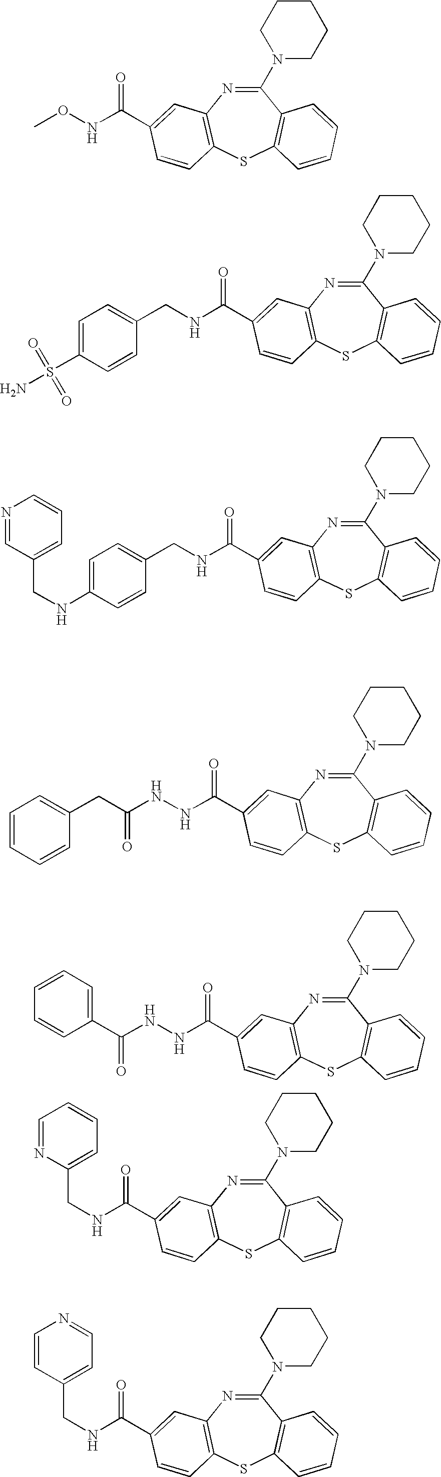 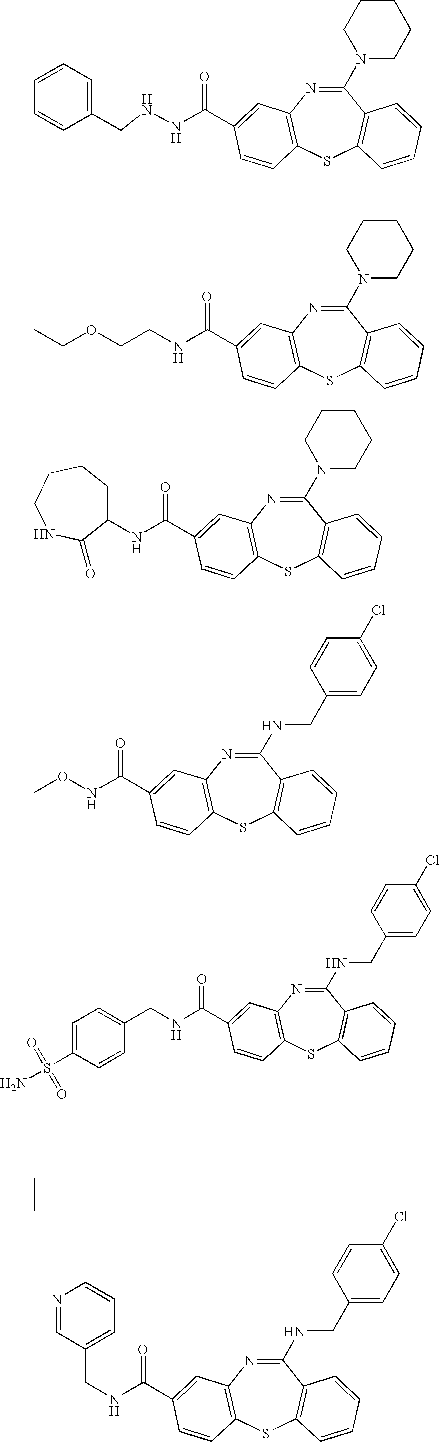 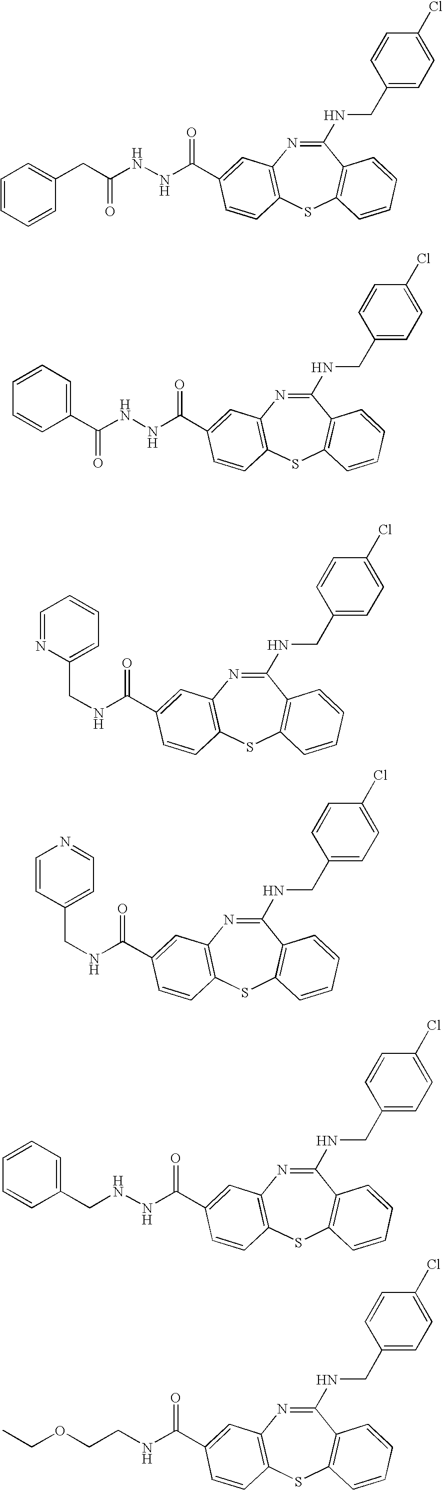 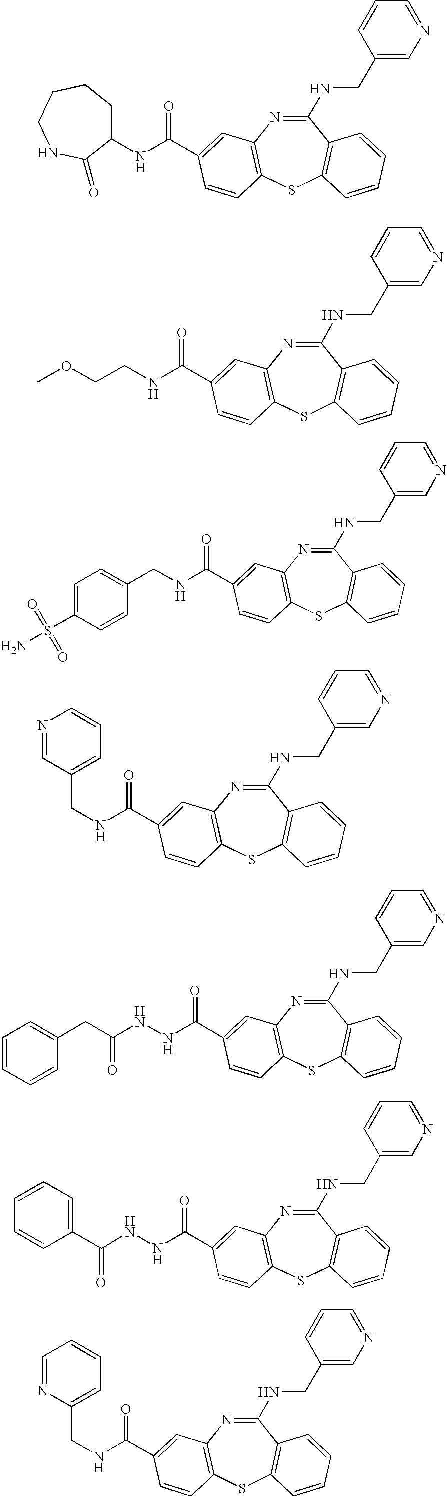 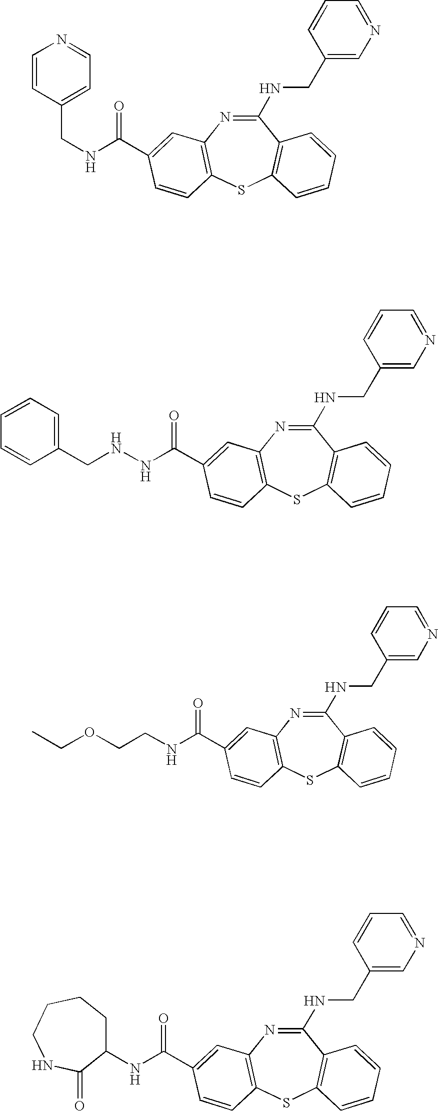
Series K

### Examples 302-391

Examples 302-391 are prepared according to the general procedure for Negishi cross-coupling starting from 10-15 mg of the appropriate amidoimidoyl chloride (represented by titled compounds in Examples 262-271) and the proper arylzinc halide (8 eq) in THF. Ammonium chloride (0.02 ml) is added to the reaction mixtures, which are then passed through a short column (Na₂SO₄/silica) using EtOAc as eluent. The eluents are concentrated at reduced pressure and the crude products are purified by preparative LC/MS. Yields are determined by weighing and purities by analytical LC/MS. 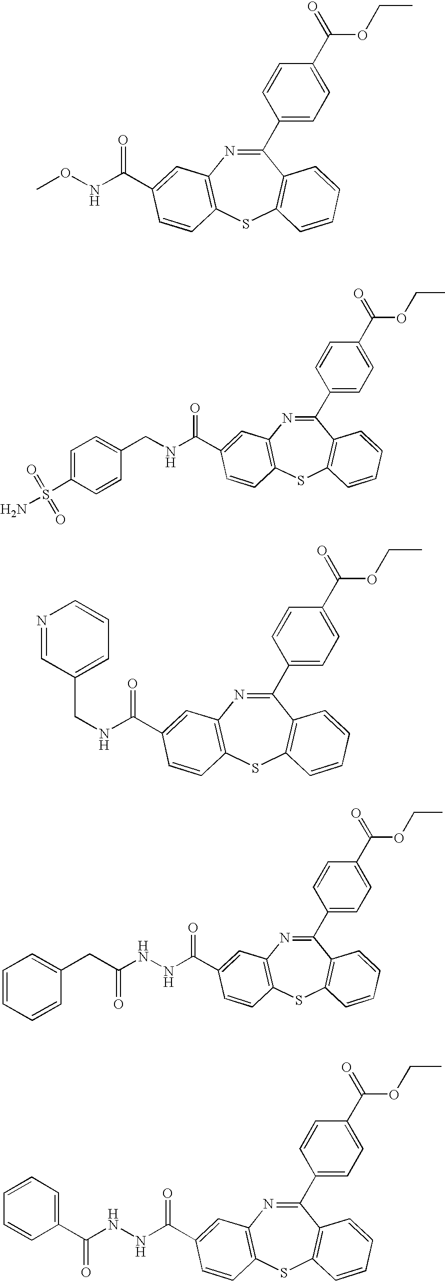 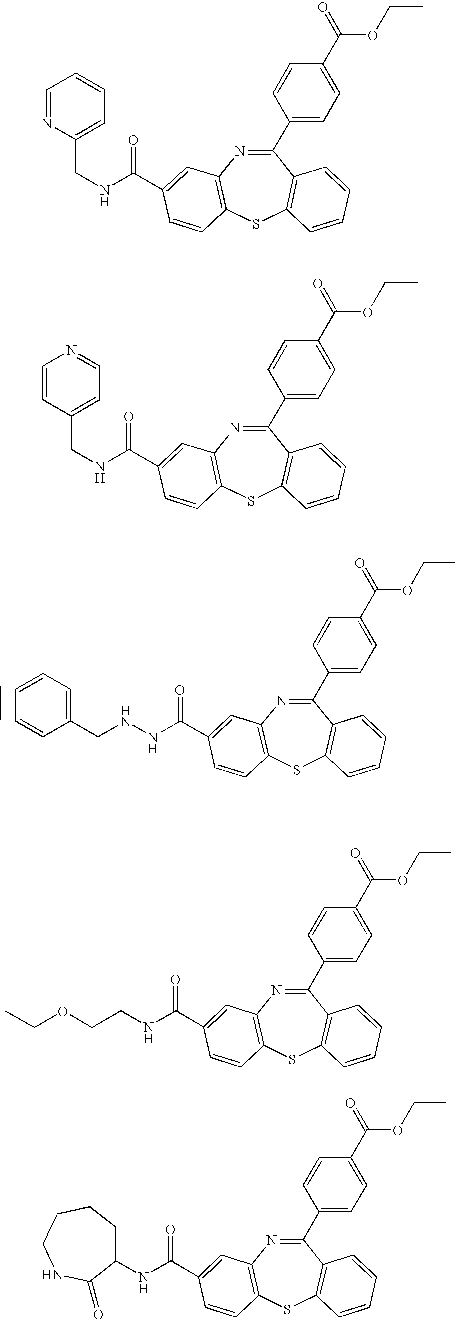 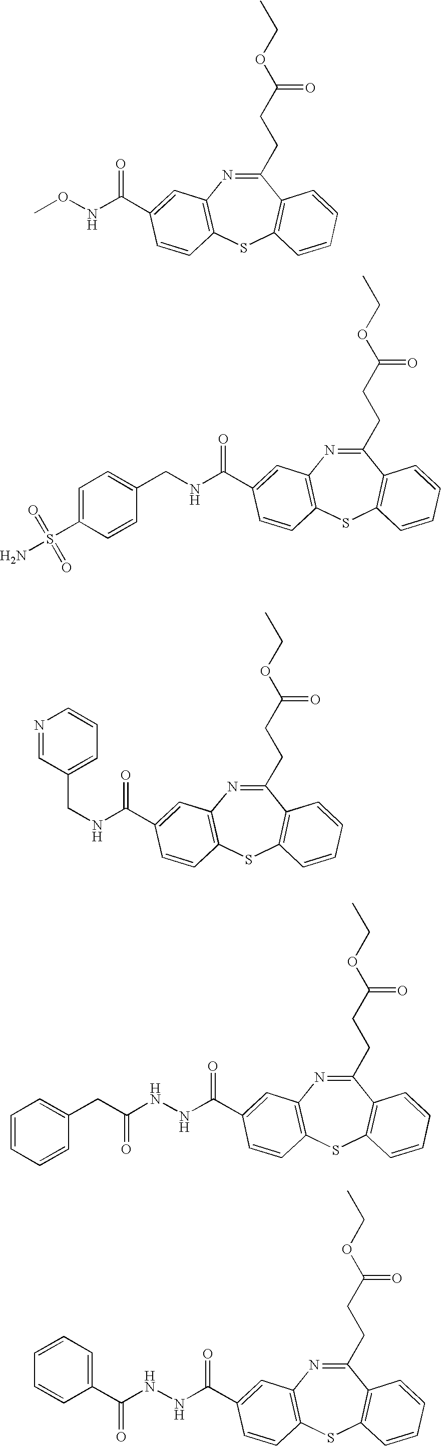 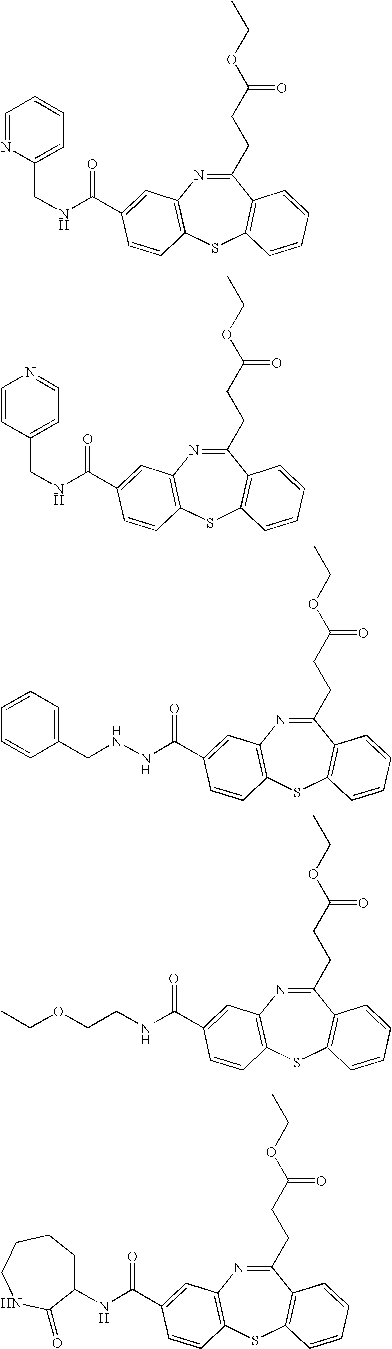 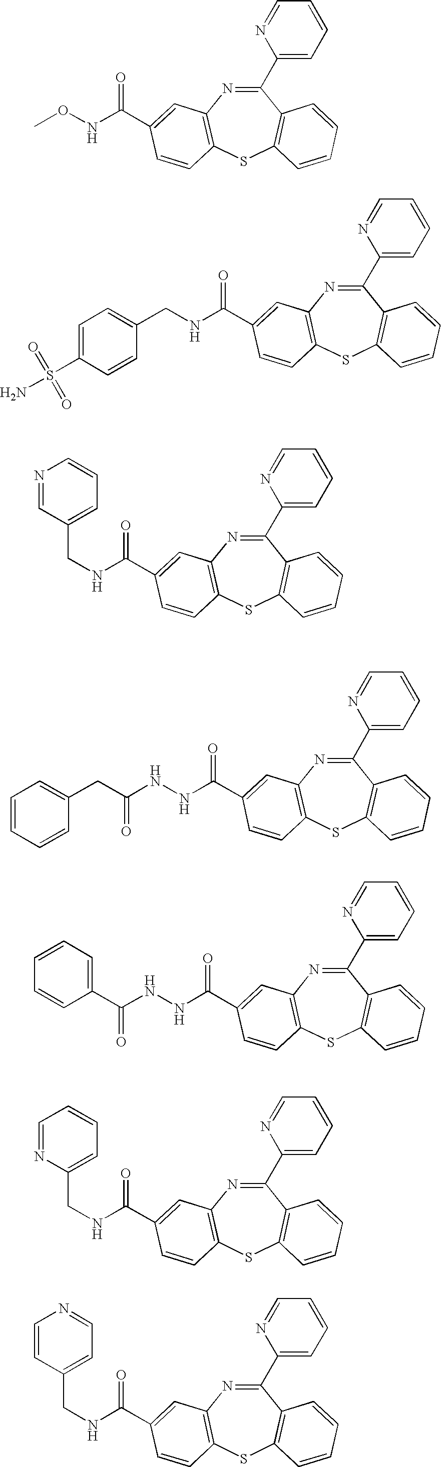 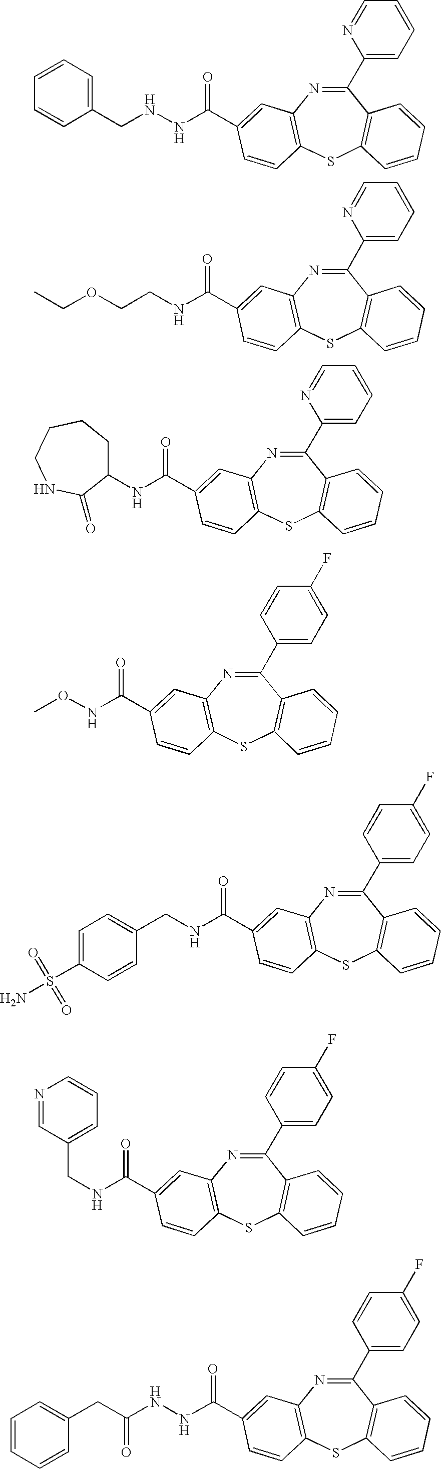 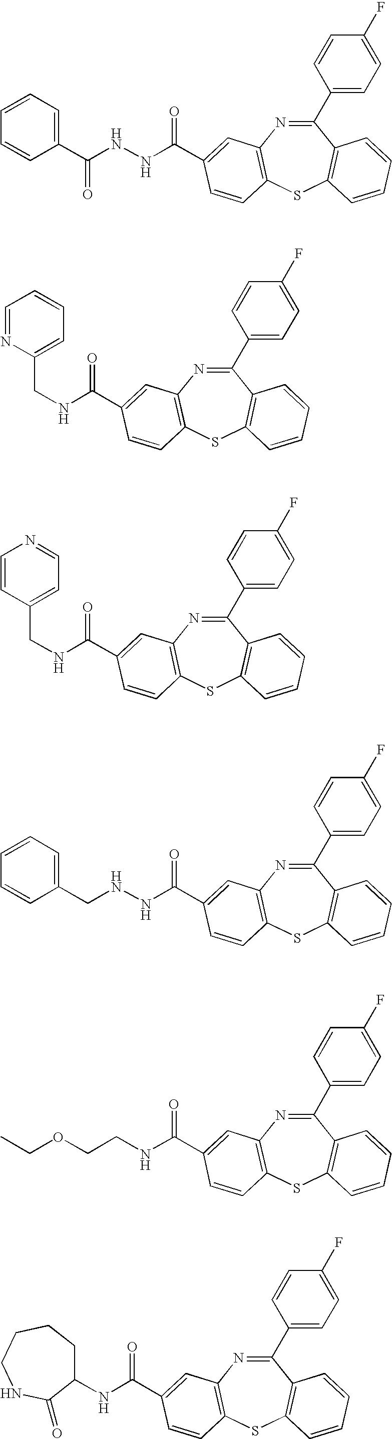 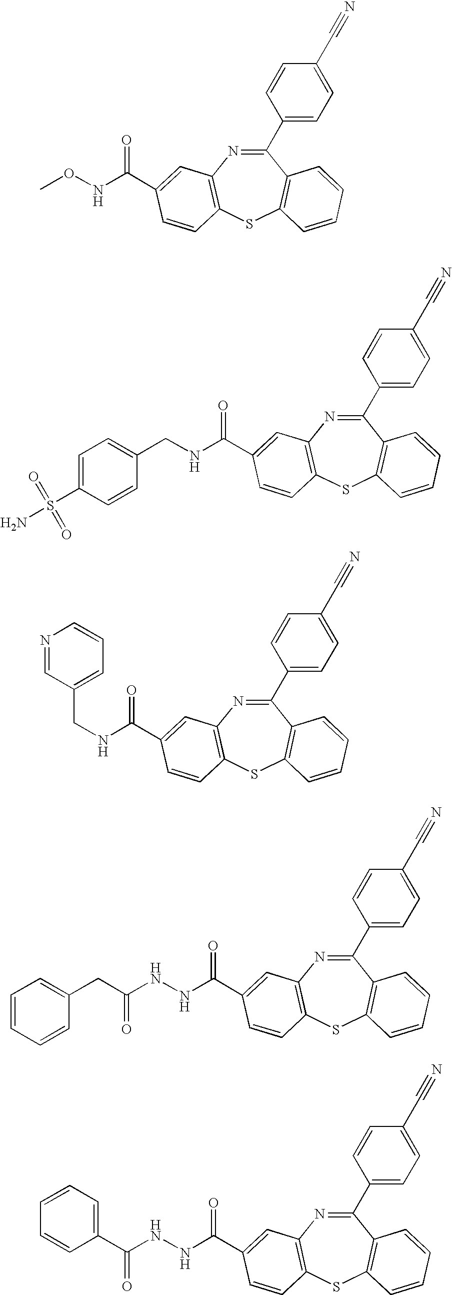 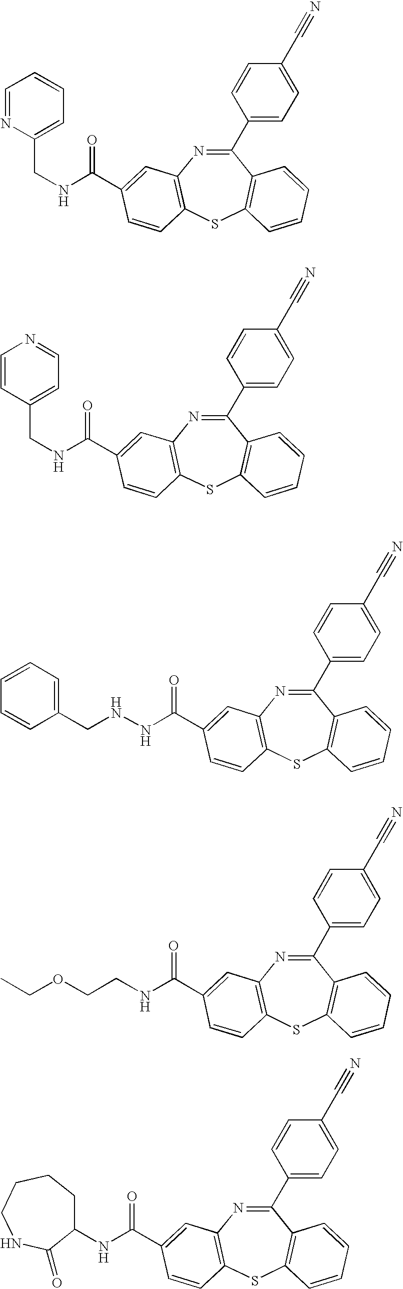 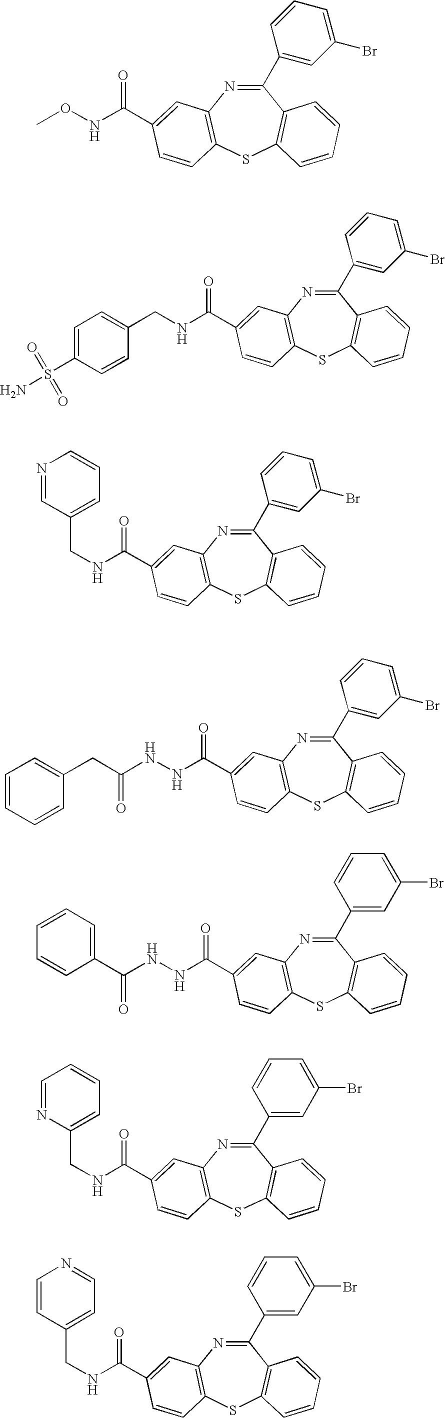 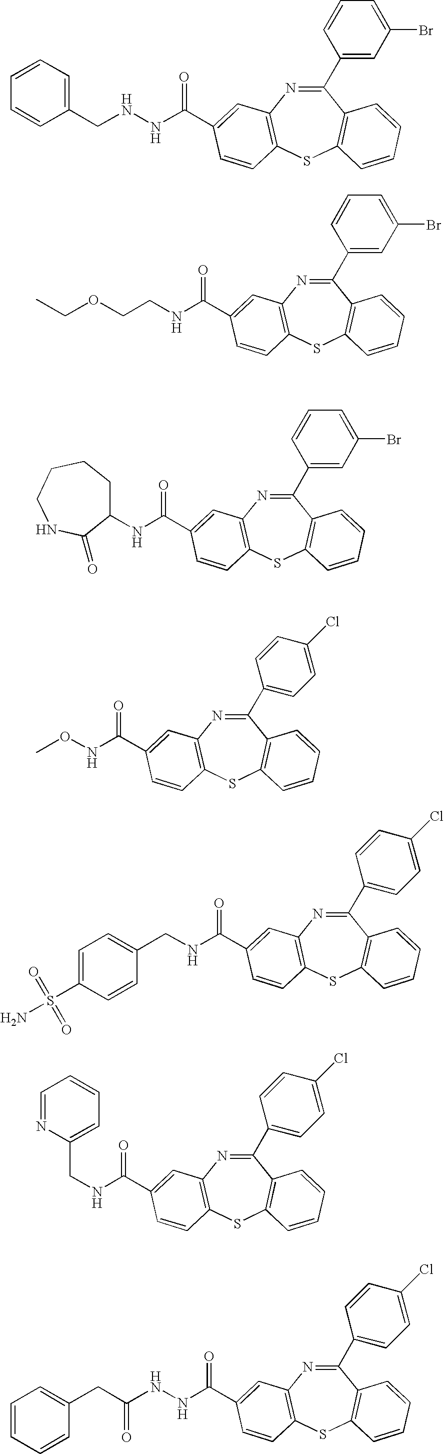 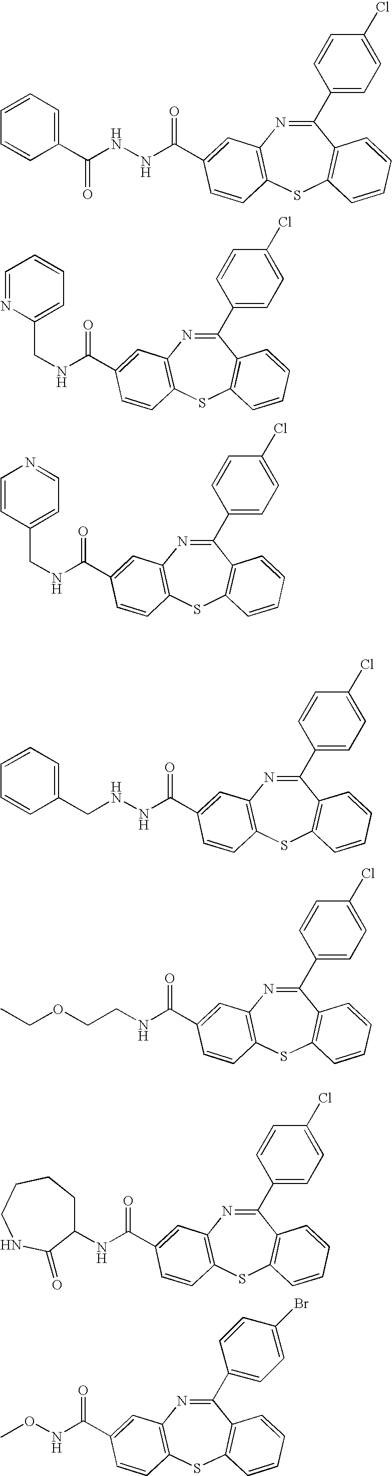 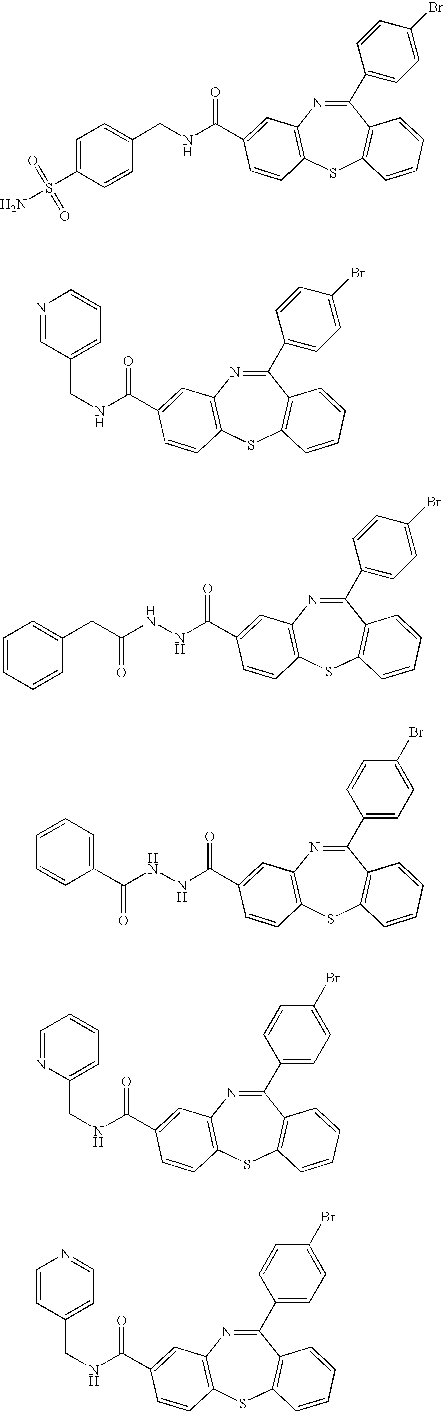 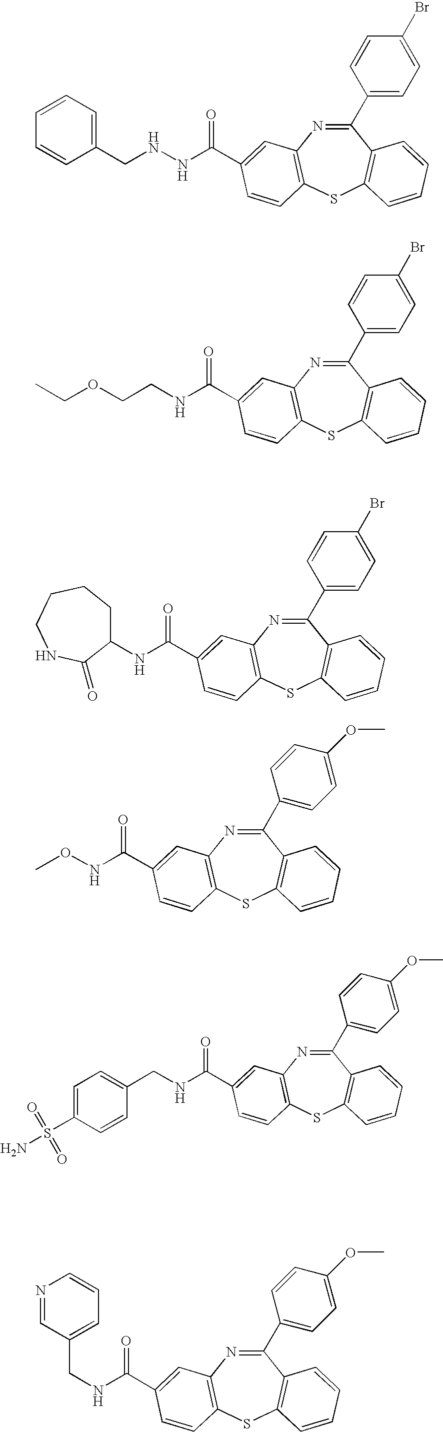 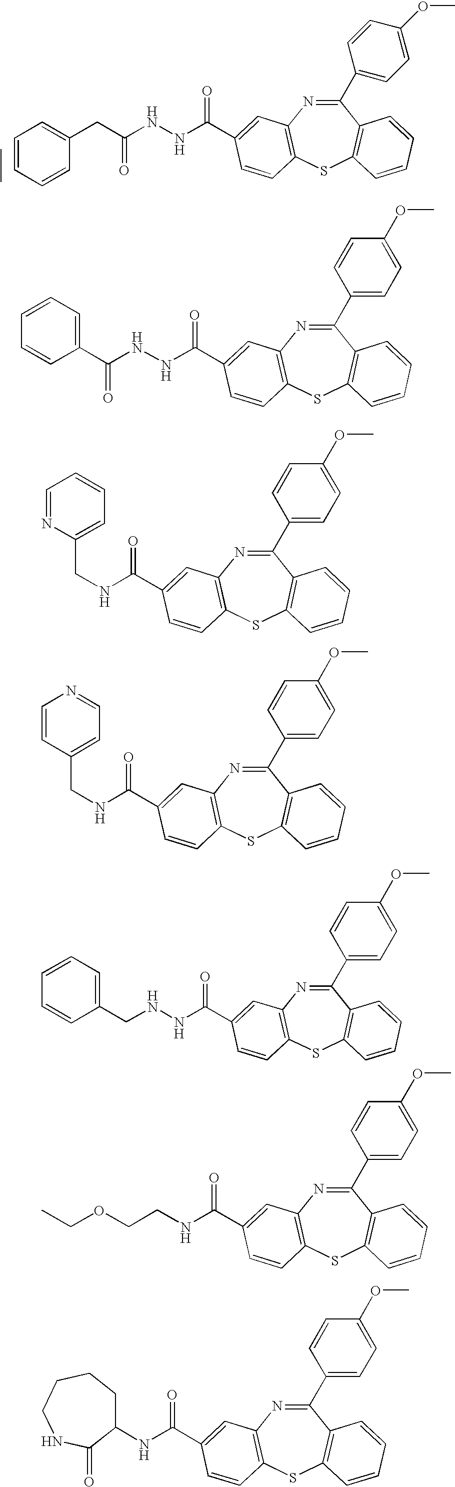
Series L

The amidoimidoyl chlorides (Examples 392-403) were synthesized according to the general procedure for amide formation using 11-chloro-dibenzo[b,f][1,4]thiazepine-8-carbonyl chloride (300 mg, 1 mmol) and the proper amine (2.5 mmol) except that the reaction mixture was passed through a pad of silica and eluted with a mixture of THF and EtOAc. The eluents were concentrated at reduced pressure and the obtained crude products were directly used in the next reactions without further purifications or characterization.

### Examples 392-403

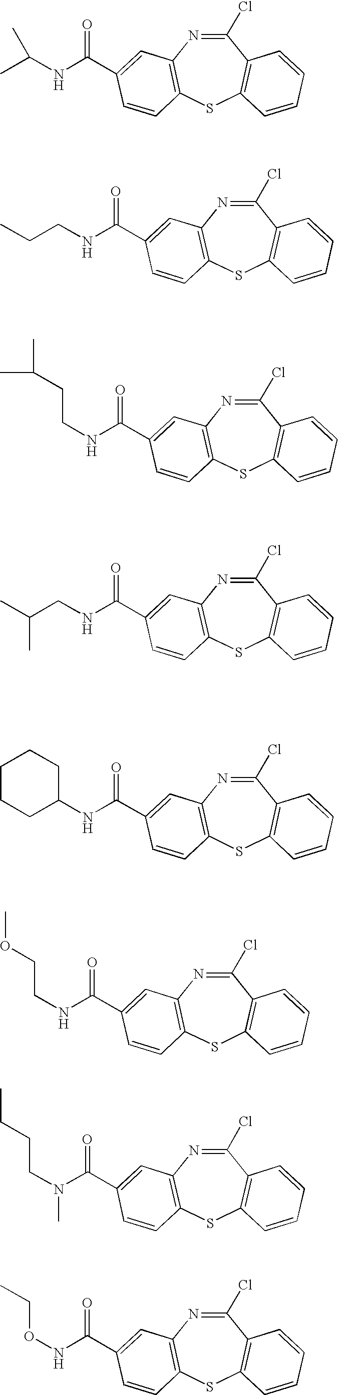 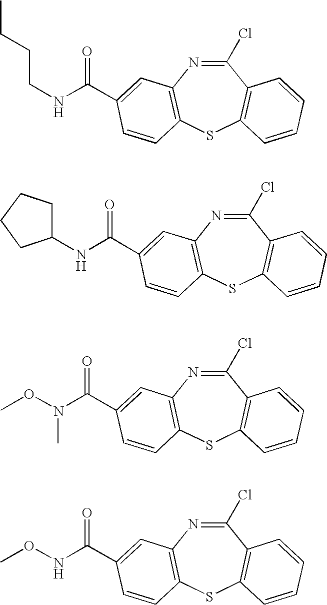 Series M

### Examples 404-499

Examples 404-499 are prepared according to the general procedure for Negishi cross-coupling starting from 10-15 mg of the appropriate amidoimidoyl chloride (represented by title compounds in Examples 392-403) and the proper arylzinc halide (8 eq) in THF.

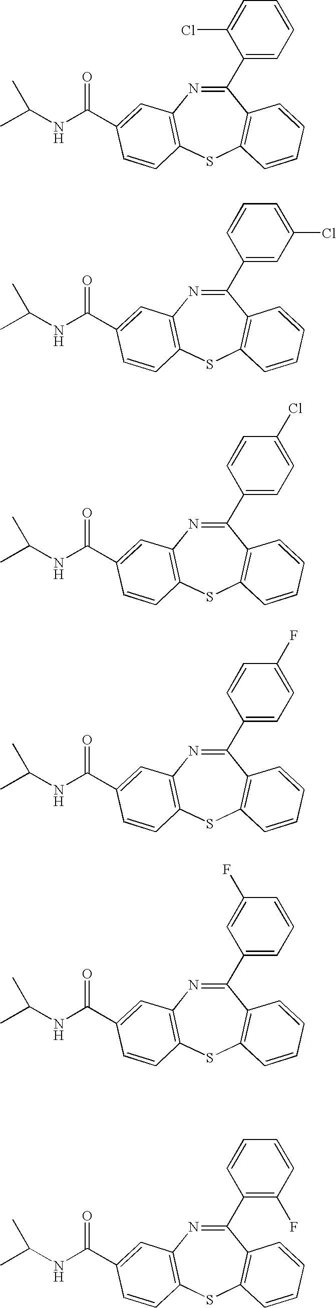

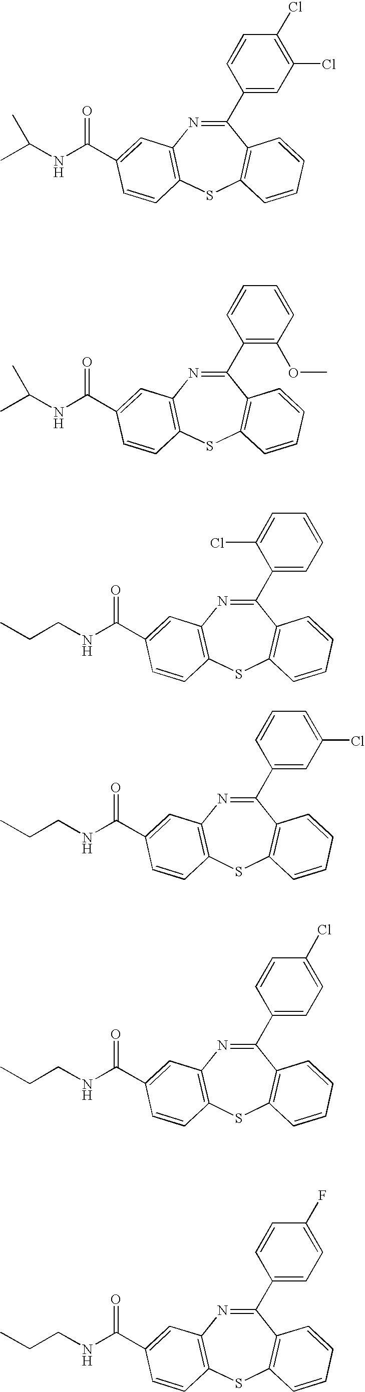

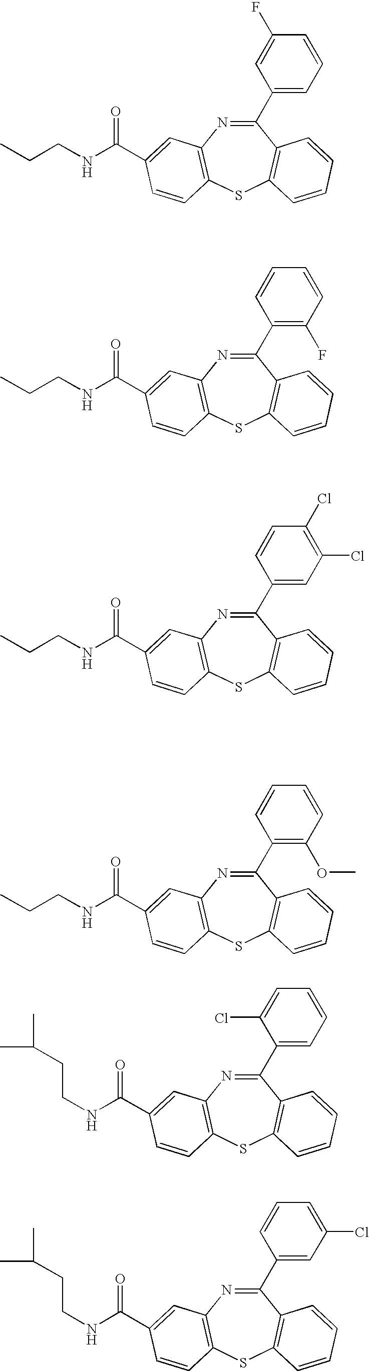

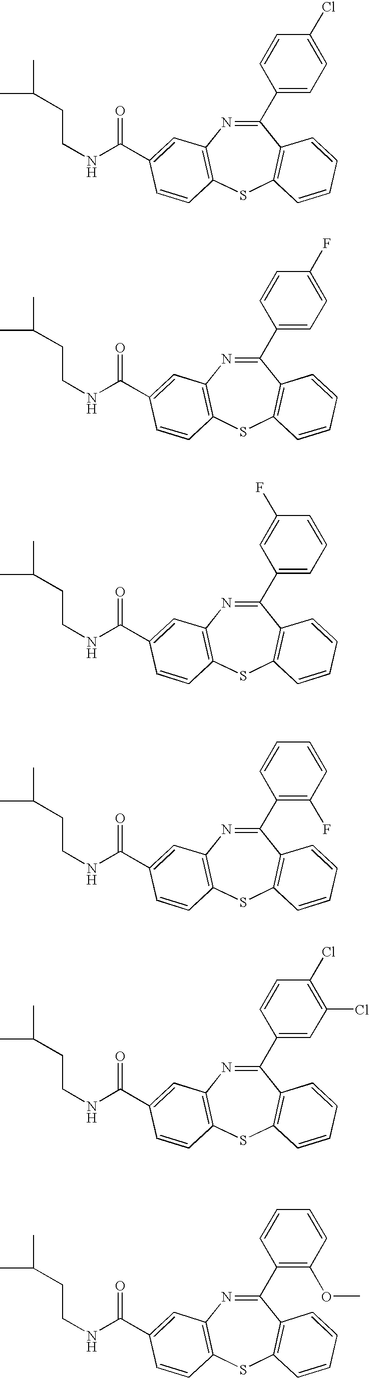

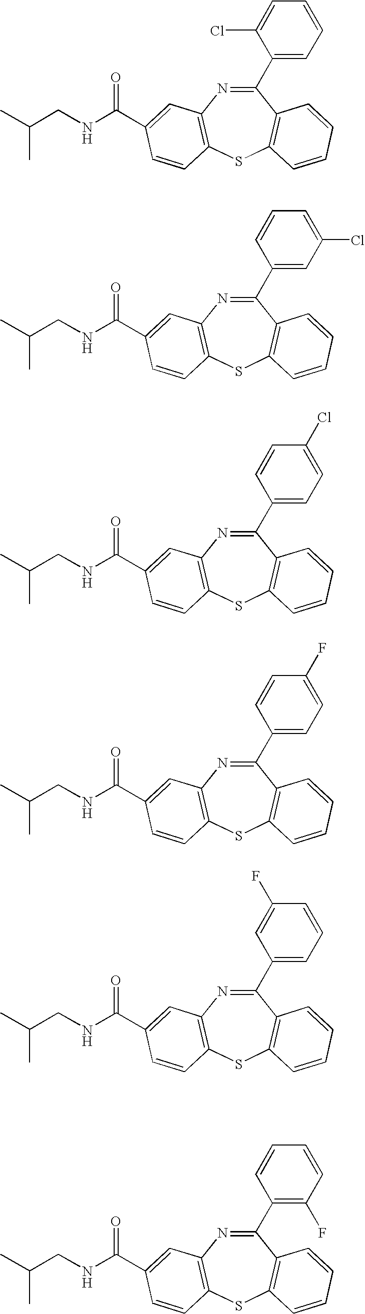

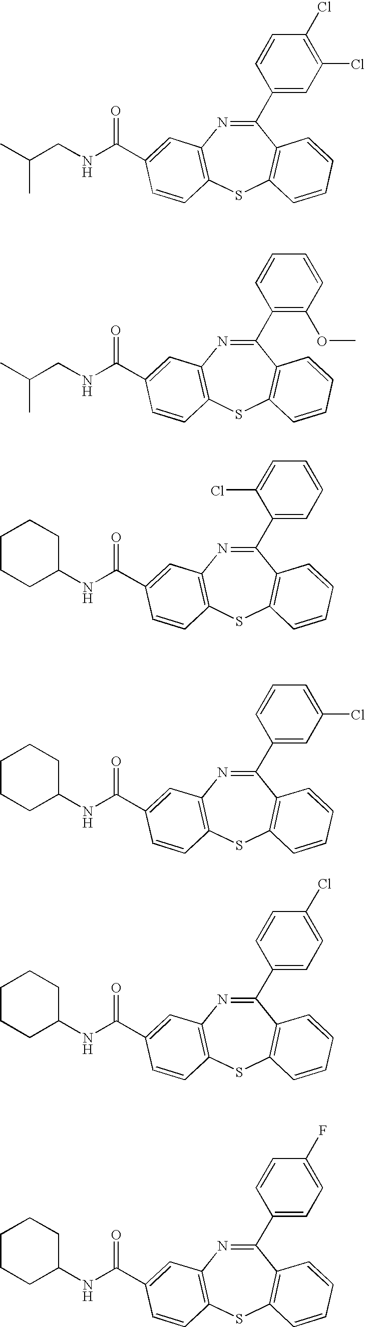

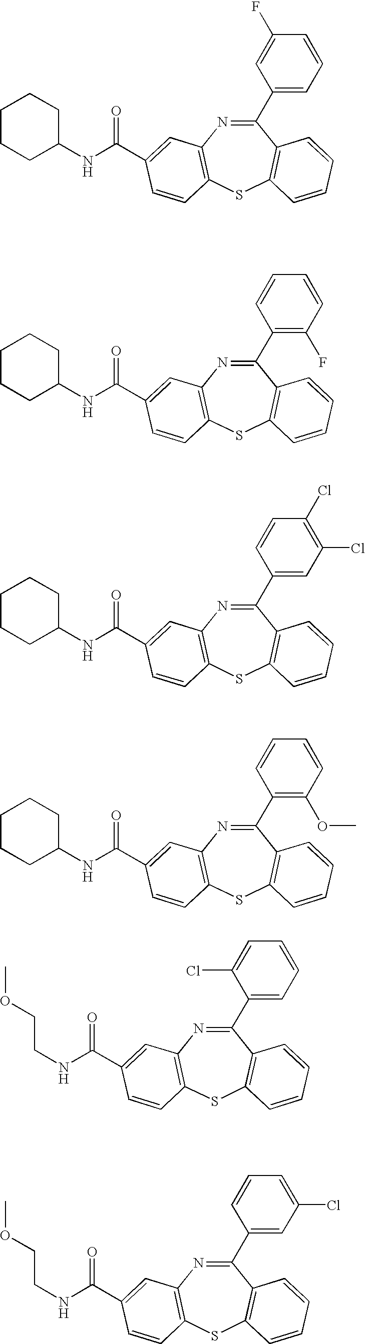

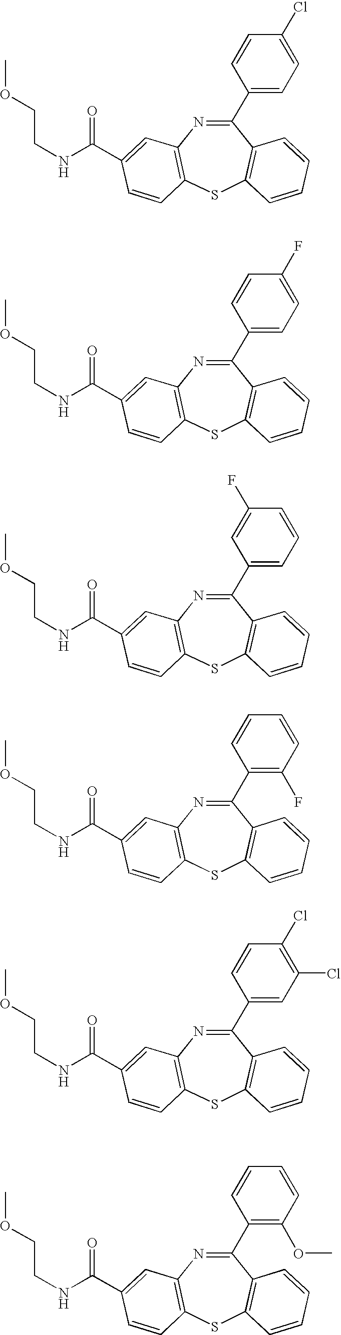

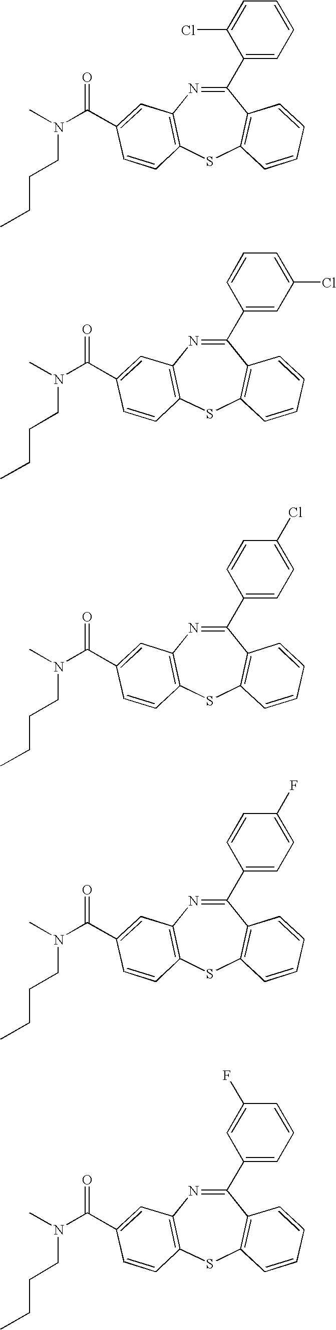

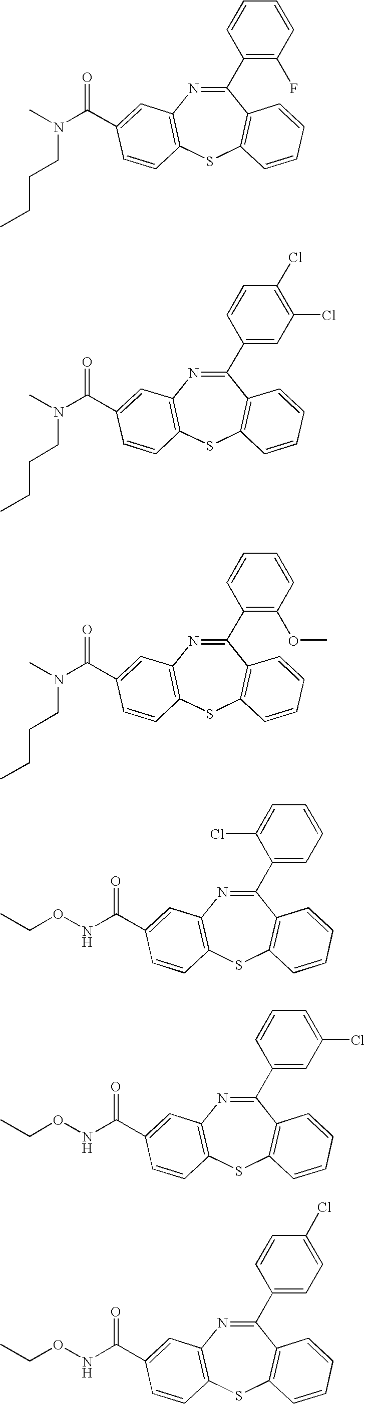

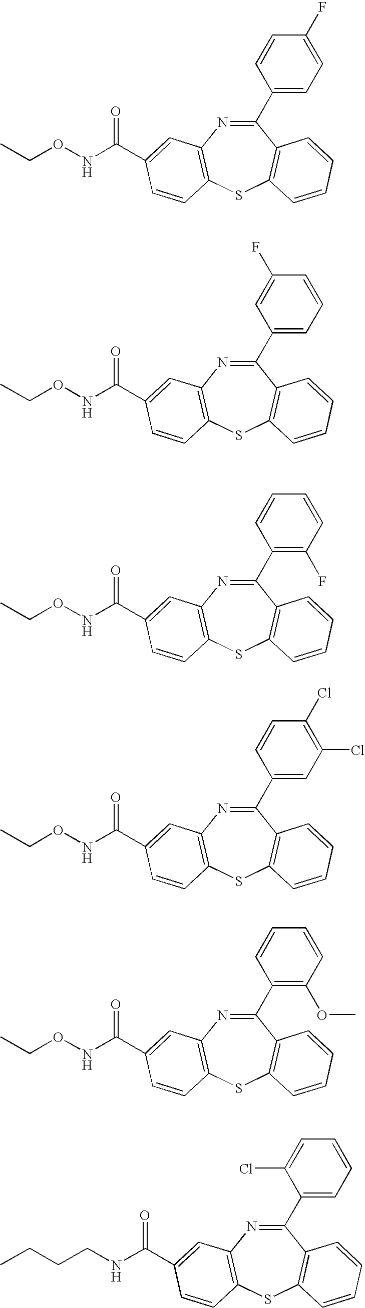

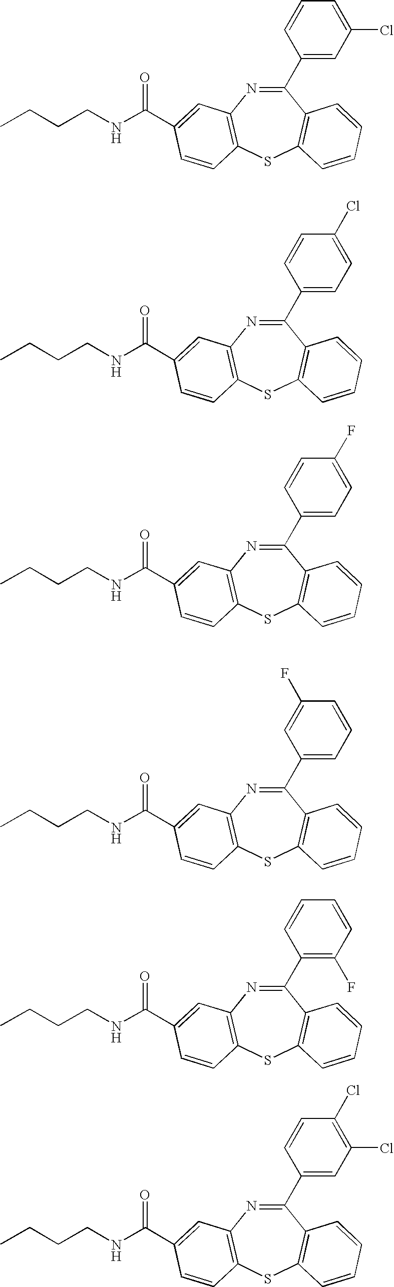

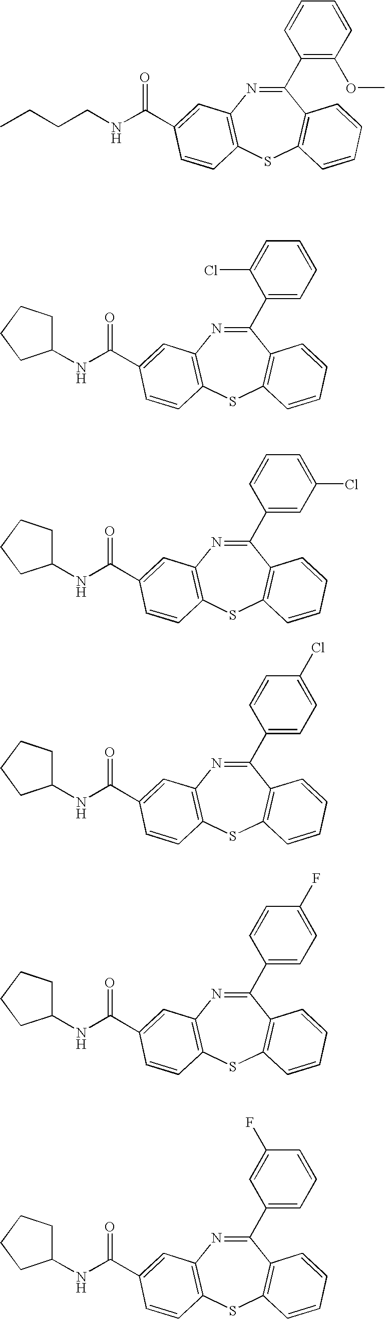

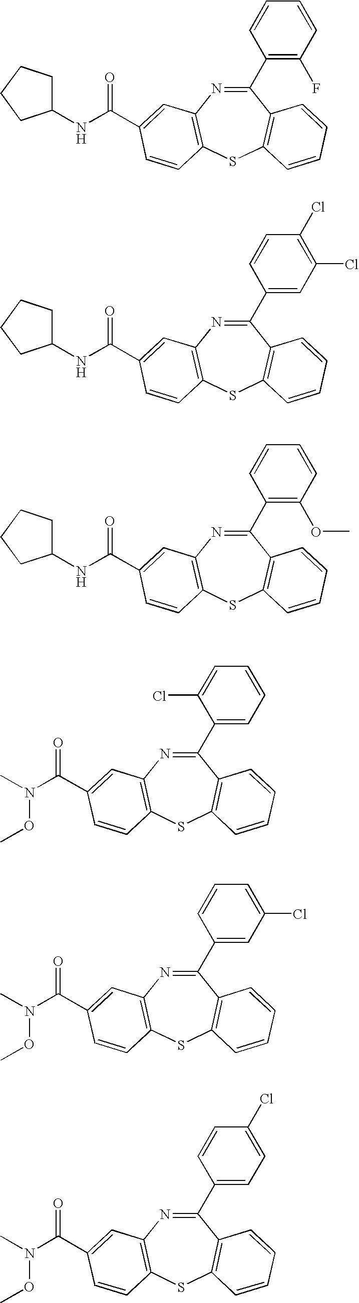

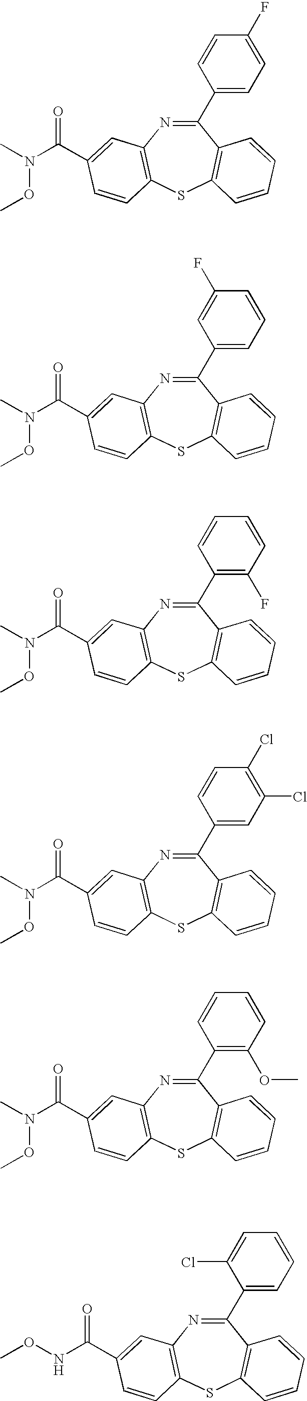

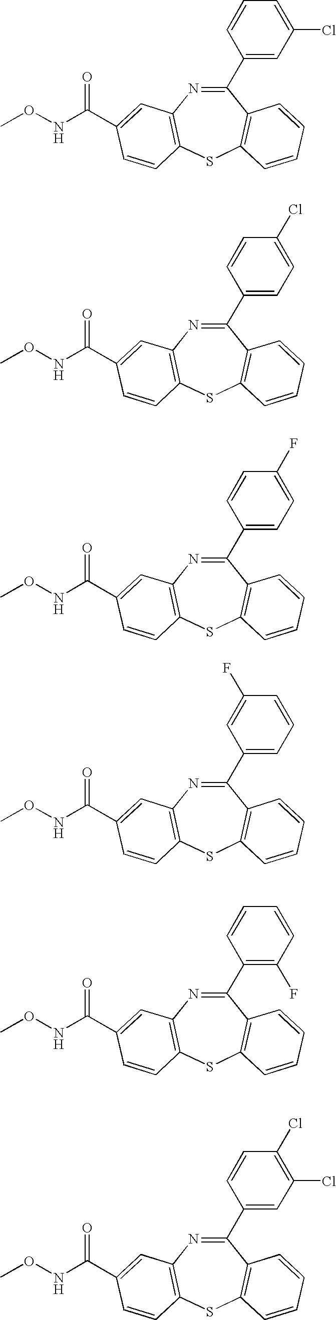

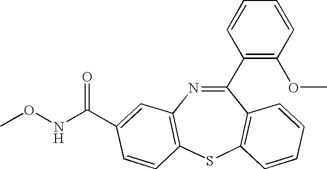

The following compounds (Examples 500-533) were synthesised from 11-chloro-dibenzo[b,f][1,4]thiazepine-8-carbonyl chloride according to the general procedure for amide formation using the proper amide followed by the general procedure for palladium catalyzed Negishi cross-coupling of amidoimidoyl chlorides and arylzinc halides or the general procedure for synthesis of amidines.

### Example 500

### (E)-11-(5-chlorothiophen-2-yl)-N-propyldibenzo[b,f][1,4]thiazepine-8-carboxamide

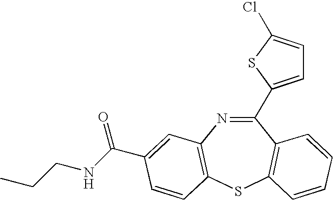

Amount isolated: 2.5 mg. LCMS m/z [M+H]⁺: 413, purity (UV/MS): 100/98, t_{R}=5.60 min.

### Example 501

### (Z)-11-(4-chloro-2-fluorophenyl)-N-isobutyldibenzo[b,f][1,4]thiazepine-8-carboxamide

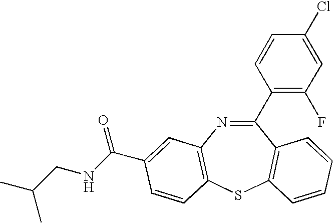

Amount isolated: 88 mg (28%).

¹H NMR (400 MHz, CDCl₃) δ 7.90 (t, 1H, J=8.4, ArH), 7.67 (t, 1H, J=0.9, ArH), 7.55-7.51 (m, 2H, ArH), 7.41 (dt, 1H, J=1.6, 7.6, ArH), 7.32-7.24 (m, 3H, ArH), 7.14-7.08 (m, 2H, ArH), 6.12 (br s, 1H, NH), 3.28 (t, 2H, J=6.8, NCH₂), 1.87 (sept, 1H, J=6.8, CH_{iBu}), 0.97 (d, 6H, J=6.8, 2×CH₃). LCMS m/z [M+1]⁺: 439, purity (UV/MS): 100/95, t_{R}=5.63 min.

### Example 502

### (E)-11-(5-chlorothiophen-2-yl)-N-isobutyldibenzo[b,f][1,4]thiazepine-8-carboxamide

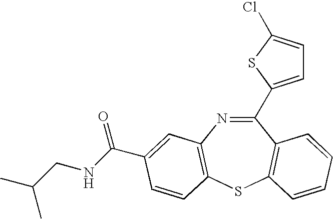

Amount isolated: 27 mg (15%).

1H NMR (400 MHz, CDCl₃) δ 7.60-7.34 (m, 7H, ArH), 6.94 (d, 1H, J=4.0thiophenH), 6.89 (d, 1H, J=4.0, thiopheneH), 6.15 (br m, 1H, NH), 3.26 (dd, 2H, J=6.4, 7.2, CH_{2iBu}), 1.87 (m, 1H, CH_{iBu}), 0.96 (d, 6H, J=6.8, 2×CH₃). ¹³C NMR (100 MHz, CDCl₃), δ 166.8, 162.7, 148.5, 145.1, 140.5, 137.1, 136.2, 135.3, 133.0, 132.8, 132.1, 132.0, 131.9, 130.3, 128.4, 127.3, 124.7, 123.9, 47.6, 28.8, 20.4. LCMS m/z [M+1]⁺: 427, purity UV/MS): 66/98, t_{R}=5.83 min.

### Example 503

### (E)-N-butyl-11-(5-chlorothiophen-2-yl)dibenzo[b,f][1,4]thiazepine-8-carboxamide

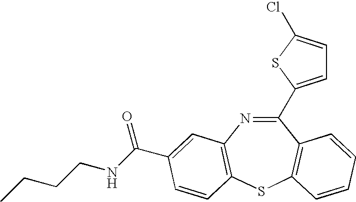

Amount isolated: 1.2 mg. LCMS m/z [M+H]⁺: 427, purity (UV/MS): 100/86, t_{R}=5.96 min.

### Example 504

### (E)-N-(3-chlorobenzyl)-11-(4-fluoropiperidin-1-yl)dibenzo[b,f][1,4]thiazepine-8-carboxamide

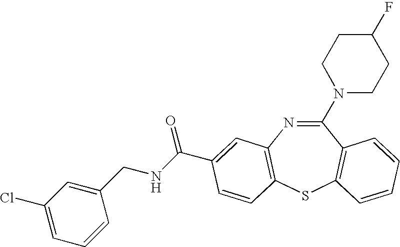

Amount isolated: 8 mg. LCMS m/z [M+H]⁺: 480, t_{R}=5.23 min.

### Example 505

### (Z)-N-(azepan-1-yl)-11-(3-chlorophenyl)dibenzo[b,f][1,4]thiazepine-8-carboxamide

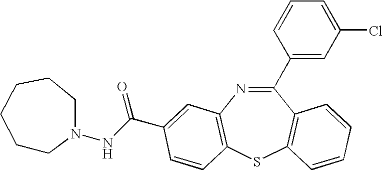

Amount isolated: 2.5 mg. LCMS m/z [M+H]⁺: 462, purity (UV/MS): 100/94, t_{R}=5.23 min.

### Example 506

### (Z)-N-((2S,6R)-2,6-dimethylpiperidin-1-yl)-11-(3-flourophenyl)dibenzo[b,f][1,4]thiazepine-8-carboxamide

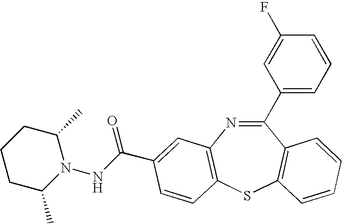

Amount isolated: 4.1 mg. LCMS m/z [M+H]⁺: 460, purity (UV/MS):100/61, t_{R}=4.89 min.

### Example 507

### (Z)-11-(3,4-dichlorophenyl)-N-((2S,6R)-2,6-dimethylpiperidin-1-yl)dibenzo[b,f][1,4]thiazepine-8-carboxamide

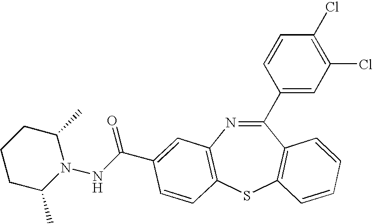

Amount isolated: 0.7 mg. LCMS m/z [M+H]⁺: 510, purity (UV/MS):100/100, t_{R}=5.68 min.

### Example 508

### (E)-N-isobutyl-11-(3-methylthiophen-2-yl)dibenzo[b,f][1,4]thiazepine-8-carboxamide

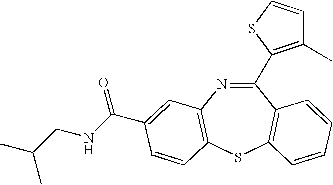

Amount isolated: 9.1 mg. LCMS m/z [M+H]⁺: 407, purity (UV/MS): 100/98, t_{R}=9.55 min.

### Example 509

### (Z)-N-(3-chlorophenethyl)-11-(3-chlorophenyl)dibenzo[b,f][1,4]thiazepine-8-carboxamide

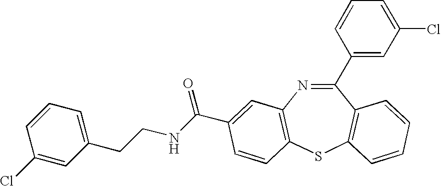

Amount isolated: 10 mg. LCMS m/z [M+H]⁺587, t_{R}=6.28 min.

### Example 510

### (Z)-11-(4-bromophenyl)-N-isobutyldibenzo[b,f][1,4]thiazepine-8-carboxamide

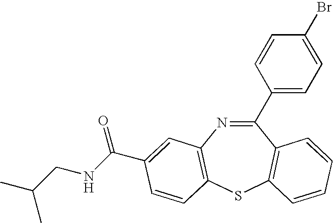

Amount isolated: 1.1 mg. LCMS m/z [M+H]⁺: 465, purity (UV/MS): 100/65, t_{R}=5.97 min.

### Example 511

### (Z)-N-isobutyl-11-(4-methoxyphenyl)dibenzo[b,f][1,4]thiazepine-8-carboxamide

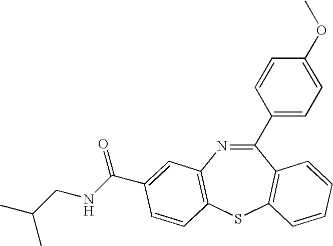

Amount isolated: 3.5 mg. LCMS m/z [M+H]⁺: 417, purity (UV/MS): 100/98, t_{R}=5.35 min.

### Example 512

### (Z)-11-(4-fluorophenyl)-N-(piperidin-1-yl)dibenzo[b,f][1,4]thiazepine-8-carboxamide

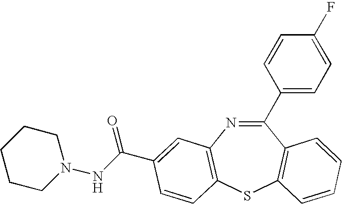

Amount isolated: 0.6 mg. LCMS m/z [M+H]⁺: 432, purity (UV/MS):98/93, t_{R}=4.41 min.

### Example 513

### (Z)-11-(3-chlorophenyl)-N-((2S,6R)-2,6-dimethylpiperidin-1-yl)dibenzo[b,f][1,4]thiazepine-8-carboxamide

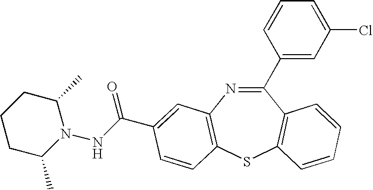

Amount isolated: 10.7 mg. LCMS m/z [M+H]⁺: 476, purity (UV/MS): 100/54, t_{R}=5.24 min.

### Example 514

### (Z)-11-(4-chloro-2-fluorophenyl)-N-propyldibenzo[b,f][1,4]thiazepine-8-carboxamide

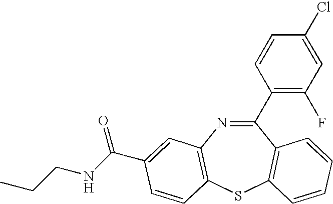

Amount isolated: 4.4 mg. LCMS m/z [M+H]⁺: 425, purity (UV/MS): 100/94, t_{R}=9.56 min.

### Example 515

### (E)-N-butyl-11-(3-methylthiophen-2-yl)dibenzo[b,f][1,4thiazepine-8-carboxamide

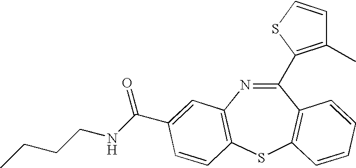

Amount isolated: 6.3 mg. LCMS m/z [M+H]⁺: 407, purity (UV/MS): 100/100, t_{R}=9.63 min.

### Example 516

### (Z)-N-(azepan-1-yl)-11-(3-fluorophenyl)dibenzo[b,f][1,4]thiazepine-8-carboxamide

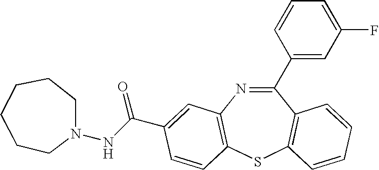

Amount isolated: 2.3 mg. LCMS m/z [M+H]⁺: 446, purity (UV/MS): 97/64, t_{R}=4.85 min.

### Example 517

### (Z)-N-butyl-11-(4-methoxyphenyl)dibenzo[b,f][1,4]thiazepine-8-carboxamide

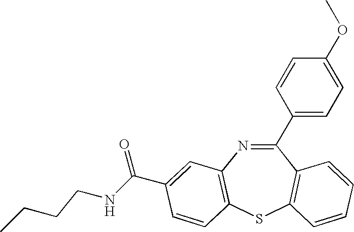

Amount isolated: 2.8 mg. LCMS m/z [M+H]⁺: 417, purity (UV/MS): 90/94, t_{R}=5.25 min.

### Example 518

### (Z)-11-(3-chlorophenyl)-N-(2-(pyridin-2-yl)ethyl)dibenzo[b,f][1,4]thiazepine-8-carboxamide

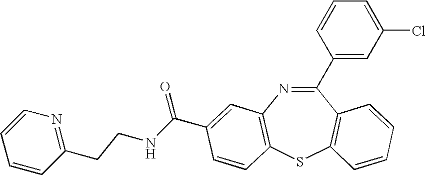

Amount isolated: 2.6 mg. LCMS m/z [M+H]⁺: 470, purity (UV/MS): 100/97, t_{R}=4.67 min.

### Example 519

### (E)-N-butyl-11-(pyridin-2-yl)dibenzo[b,f][1,4]thiazepine-8-carboxamide

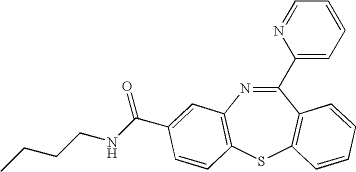

Amount isolated: 4.1 mg. LCMS m/z. [M+H⁺: 388, purity (UV/MS): 100/92, t_{R}=4.08 min.

### Example 520

### (Z)-11-(4-methoxyphenyl)-N-propyldibenzo[b,f][1,4]thiazepine-8-carboxamide

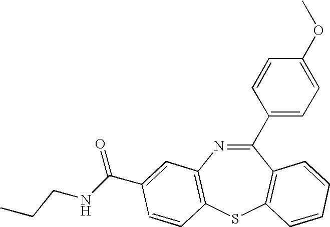

Amount isolated: 0.9 mg. LCMS m/z [M+H⁺: 403, purity (UV/MS): 93/100, t_{R}=4.⁹⁵ min.

### Example 521

### (E)-11-(3-methylthiophen-2-yl)-N-propyldibenzo[b,f][1,4thiazepine-8-carboxamide

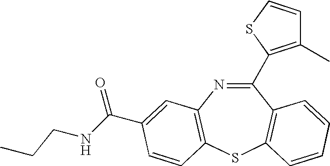

Amount isolated: 5.7 mg. LCMS m/z [M+H⁺: 393, purity (UV/MS): 100/93, t_{R}=9.01 min.

### Example 522

### (Z)-11-(3-fluorophenyl)-N-(piperidin-1-yl)dibenzo[b,f][1,4]thiazepine-8-carboxamide

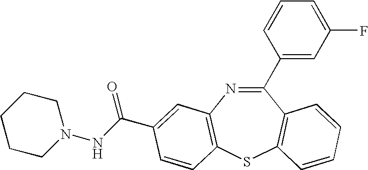

Amount isolated: 2.8 mg. LCMS m/z [M+H⁺: 432, purity (UV/MS): 100/78, t_{R}=4.47 min.

### Example 523

### (Z)-N-((2S,6R)-2,6-dimethylpiperidin-1-yl)-l 1-(4-fluorophenyl)dibenzo[b,f]1,4]thiazepine-8-carboxamide

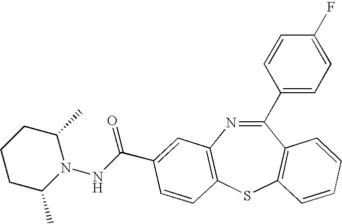

Amount isolated: 0.6 mg. LCMS m/z [M+H⁺: 460, purity (UV/MS): 98/91, t_{R}=4.79 min.

### Example 524

### (E)-N-isopentyl-11-(3-methylthiophen-2-yl)dibenzo[b,f][1,4]thiazepine-8-carboxamide

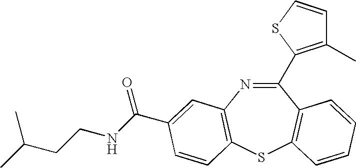

Amount isolated: 6.7 mg. LCMS m/z [M+H⁺: 421, purity (UV/MS): 100/96, t_{R}=10.05 min.

### Example 525

### (E)-11-(5-chlorothiophen-2-yl)-N-(2-methoxyethyl)dibenzo[b,f][1,4]thiazepine-8-carboxamide

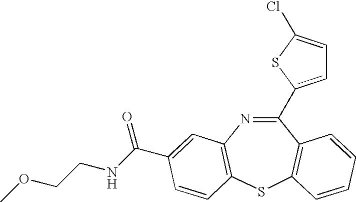

Amount isolated: 5.8 mg. LCMS m/z [M+H⁺: 429, purity (UV/MS): 100/93, t_{R}=5.01 min.

### Example 526

### (Z)-N-isopentyl-11-(4-methoxyphenyl)dibenzo[b,f][1,4]thiazepine-8-carboxamide

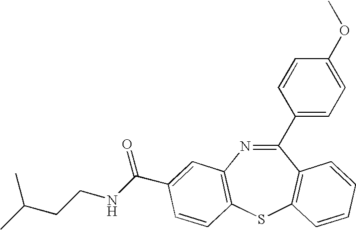

Amount isolated: 3.9 mg. LCMS m/z [M+H⁺: 431, purity (UV/MS): 100/100, t_{R}=5.67 min.

### Example 527

### (E)-N-isopently-11-(pyridin-2-yl)dibenzo[b,f][1,4]thiazepine-8-carboxamide

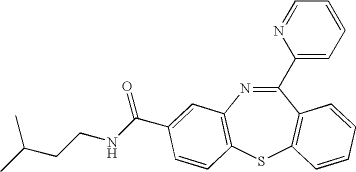

Amount isolated: 127 mg (52%).

¹H NMR (400 MHz, CDCl₃) δ 8.69-8.59 (m, 2H, ArH), 8.30-8.25 (m, 1H, ArH), 7.87-7.81 (m, 1H, ArH), 7.71 (m, 1H, ArH), 7.52 (m, 2H, ArH), 7.43-7.19 (m 4H, ArH), 6.16 (br s, 1H, NH), 3.48-3.41 (m, 2H, NCH₂), 1.66 (sept, 1H, J=6.6, CH_{iPen}), 1.48 (q, 2H, CH₂, J=6.6), 0.93 (d, 6H, J =6.6, 2×CH₃). LCMS m/z [M+H]⁺402, purity (UV/MS): 100/94. t_{R}=4.47 min.

### Example 528

### (Z)-11-(4-chlorophenyl)-N-(2-(pyridin-2-yl)ethyl)dibenzo[b,f][1,4]thiazepine-8-carboxamide

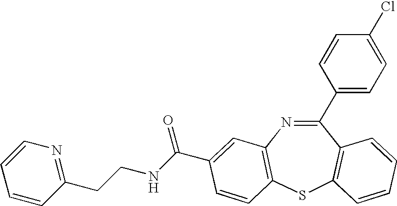

Amount isolated: 2.3 mg. LCMS m/z [M+H⁺: 470, purity (UV/MS): 100/88, t_{R}=4.68 min.

### Example 529

### (Z)-N-(azepan-1-yl)-11-(4-fluorophenyl)dibenzo[b,f][1,4]thiazepine-8-carboxamide

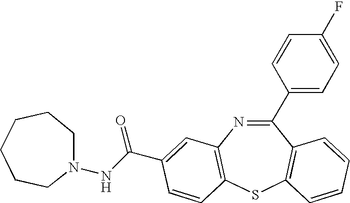

Amount isolated: 0.7 mg. LCMS m/z [M+H⁺: 446, purity (UV/MS): 98/92, t_{R}=4.80 min.

### Example 530

### (E)-N-isobutyl-11-(pyridin-2-yl)dibenzo[b,f][1,4]thiazepine-8-carboxamide

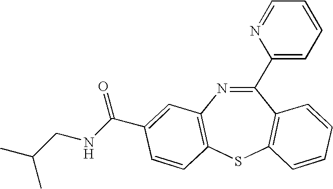

Amount isolated: 77 mg (46%).

¹H NMR (400 MHz, CDCl₃) δ 8.72-8.70 (m, 1H, ArH), 8.29-8.24 (m, 1H, ArH), 7.87 (dt, 1H, J=1.6, 7.6, ArH), 7.75 (m, 1H, ArH), 7.57-7.52 (m, 3H, ArH), 7.44-7.38 (m, 2H, ArH), 7.32 (dt, 1H, J=1.2, 7.6, ArH), 7.23-7.20 (m, 1H, ArH), 6.19 (br s, 1H, NH), 3.27 (t, 2H, J=6.4, NHCH₂), 1.88 (sept, 1H, J=6.4, CH_{iBU}), 0.97 (d, 6H, J=6.4, 2×CH₃). LCMS m/z [M+H]⁺388, purity (UV/MS): 94/60. t_{R}=4.00 min.

### Example 531

### (Z)-11-(4-bromophenyl)-N-propyldibenzo[b,f][1,4]thiazepine-8-carboxamide

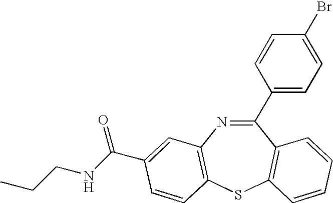

Amount isolated: 0.6 mg. LCMS m/z [M+H⁺: 451, purity (UV/MS): 100/61, t_{R}=5.65 min.

### Example 532

### (Z)-11-(3,4-dichlorophenyl)-N-(2-(pyridin-2-yl)ethyl)dibenzo[b,f][1,4]thiazepine-8-carboxamide

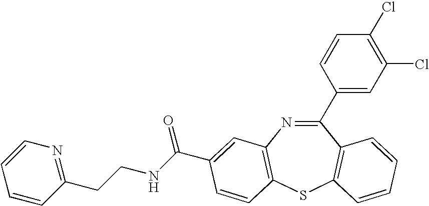

Amount isolated: 2.1 mg. LCMS m/z [M+H⁺: 504, purity (UV/MS): 100/96, t_{R}=5.15 min.

### Example 533

### (Z)-11-(4-bromophenyl)-N-(2-methoxyethyl)dibenzo[b,f][1,4]thiazepine-8-carboxamide

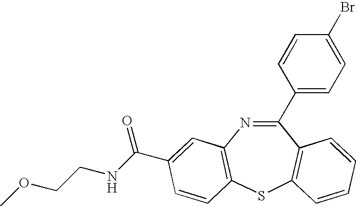

Amount isolated: 3.7 mg. LCMS m/z [M+H⁺: 467, purity (UV/MS): 100/78, t_{R}=5.09 min.

### Example 534

### 11-chloro-dibenzo[b,f][1,4]thiazepine-8-carboxylic acid methyl ester

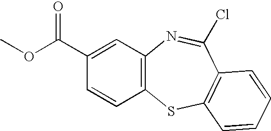

A mixture of the lactam (1 eq.) and PCd₅ (5 eq.) in toluene was heated at 110° C. for 2 hours. The reaction mixture was then cooled to room temperature and excess of PCl₅ and toluene was removed at reduced pressure using an oilpump to give crude product, which was used without further purification. The following reagents were employed: 11-oxo-10,11-dibenzo[b,f][1,4]thiazepine-8-carboxylic acid methyl ester (540 mg, 1.89 Mmol), PCl₅ (1.97 g, 9.47 mmol), toluene (15 mL). Purification by flash chromatography (ethyl acetate/heptane 1:4) afforded 410 mg (71%) of the titled compound as an yellow solid.

¹H NMR (400 MHz, CDCl₃): δ 7.86 (1H, dd, J=2.0, 0.4Hz), 7.75 (1H, dd, J=8.0, 1.6 Hz), 7.69-7.67 (1H, m), 7.45 (1H, dd, J=8.4, 0.4Hz), 7.40-7.32 (3H, m), 3.82 (3H, s). ¹³C NMR (100 MHz, CDCl₃): δ 166.2, 156.1, 146.3, 138.1, 137.9, 133.2, 133.1, 132.9, 132.4, 131.7, 130.2, 129.2, 128.1, 127.1, 52.6.

### Example 535

### 11-butyl-dibenzo[b,f][1,4]thiazepine-8-carboxylic acid methyl ester

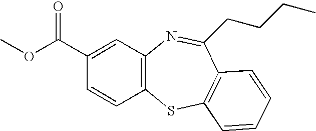

A flame dried 10 mL flask was charged under argon with the imidoyl chloride (1 eq.), Fe(acac)₃ (5 mol %) in dry THF and cooled to −40° C. Functionalized arylmagnesium halide (2 eq., 1 M in THF; prepared at −40° C.) was slowly added to the solution, keeping the temperature below −40° C. The reaction was stirred for 5 min. at −40° C., then quenched with NH₄Cl (sat., aq.) and allowed to warm to room temperature. The resulting mixture was diluted with Et₂O and the organic phase was washed with water, brine, dried (Na₂SO₃), filtered, and evaporated to give crude product. Purification by flash chromatography. The following reagents were employed: 11-chloro-dibenzo[b,f][1,4]thiazepine-8-carboxylic acid methyl ester (151.5 mg, 0.50 mmol), Fe(acac)₃ (8.85 mg, 0.05 mmol), THF (4 mL) and N-methylpyrrolidone (0.4 mL), nButyl magnesium chloride (2 M in Et₂O, 0.50 mL, 1.0 mmol). Purification by flash chromatography (ethyl acetate/heptane 1:5) afforded 144 mg (89%) of the titled compound as a yellow solid.

¹H NMR (400 MHz, CDCl₃): δ 7.84 (1H, d, J=1.6Hz), 7.68 (1H, dd, J=8.0, 1.6 Hz), 7.74-7.43 (2H, m), 7.40-7.31 (3H, m), 3.87 (3H, s), 3.02-2.85 (2H, m), 1.74-1.58 (2H, m), 1.55-1.41 (2H, m), 0.93 (3H, t, J=7.2Hz). ¹³C NMR (100 MHz, CDCl₃): δ 174.5, 166.7, 148.8, 139.7, 139.0, 134.4, 132.5, 132.3, 131.1, 130.9, 128.9, 127.9, 126.6, 126.1, 52.4, 42.2, 29.5, 22.7, 14.2.

### Example 536

### 11-butyl-dibenzo[b,f]f[1,4]thiazepine-8-carboxylic acid methoxy-methyl-amide

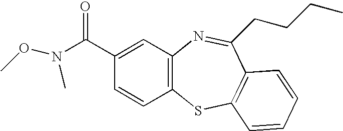

A flame dried 10 mL flask was charged under argon with the imidoyl chloride (1 eq.), Fe(acac)₃ (5 mol %) in dry THF and cooled to −40° C. Functionalized arylmagnesium halide (2 eq., 1 M in THF; prepared at −40° C.) was slowly added to the solution, keeping the temperature below −40° C. The reaction was stirred for 5 min. at −40° C., then quenched with NH₄Cl (sat., aq.) and allowed to warm to room temperature. The resulting mixture was diluted with Et₂O and the organic phase was washed with water, brine, dried (Na₂SO₃), filtered, and evaporated to give crude product. Purification by flash chromatography. The following reagents were employed: 11-chloro-dibenzo[b,f][1,4]thiazepine-8-carboxylic acid methoxy-methyl-amide (61.5 mg, 0.19 mmol), Fe(acac)₃ (3.53 mg, 0.001 mmol), THF (2 mL) and N-methylpyrrolidone (0.20 mL), n-Butyl magnesium chloride (2 M in Et₂O, 0.11 mL, 0.23 mmol). Purification by flash chromatography (ethyl acetate/heptane 1:1) afforded 47 mg (70%) of the titled compound as a yellow oil.

NMR (400 MHz, CDCl₃): δ 7.45-7.42 (3H, m), 7.39-7.29 (4H, m), 3.54 (3H,s), 3.32 (3H,s), 3.01-2.82 (2H, m), 1.69-1.59 (2H, m), 1.51-1.41 (2H, m), 0.92 (3H, t, J=7.2Hz). ¹³C NMR (100 MHz, CDCl₃): δ 174.4, 169.2, 148.7, 140.0, 139.0, 135.2, 132.2, 132.1, 131.6, 130.8, 128.7, 127.9, 125.1, 124.9, 61.4, 42.2, 34.1, 29.6, 22.7, 14.1.

### Example 537

### (11-butyl-dibenzo[b,f][1,4]thiazepine-8-yl)-cyclohexyl-methanone

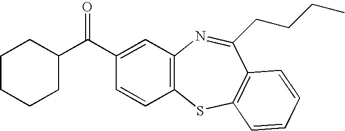

A flame dried 10 mL flask was charged under argon with 11-butyl-dibenzo[b,f][1,4]thiazepine-8-carboxylic acid methoxy-methyl-amide (29 mg, 0.08 mmol) in dry THF (2 mL) and cyclohexyl magnesium chloride (2 M in Et₂O, 0.12 mL, 0.24 mmol) was then added. The resulting reaction mixture was stirred at room temperature for 1 hour and was then diluted with ether. The organic phase was washed with water, brine, dried (Na2SO₃), filtered and evaporated to give crude product. Purification by prepatory TLC (ethyl acetate/heptane 1:10) afforded 5 mg (17%) of the titled compound as a colorless oil.

¹H NMR (400 MHz, CDCl₃): δ 7.70 (1H, d, J=2 Hz), 7.60 (1H, dd, J=8.0, 2.0 Hz), 7.49-7.44 (2H, m), 7.41-7.33 (3H,m), 3.19 (1H, tt, J=11.2, 3.2 Hz), 3.04-2.97 (1H, m), 2.92-2.84 (1H, m), 1.83-1.79 (3H, m), 1.72-1.62 (3H, m), 1.51-1.21 (8H, m), 0.93 (3H, t, J=7.6 Hz). ¹³C NMR (100 MHz, CDCl₃): δ 203.4, 174.7, 148.9, 139.8, 138.9, 137.3, 134.2, 132.8, 132.3, 130.9, 128.9, 127.9, 125.3, 124.9, 45.8, 42.3, 29.6, 29.5, 26.1, 26.0, 22.7, 14.2.

### Example 538

### 1-(11-chloro-dibenzo[b,f][1,4]thiazepine-8-yl)-pentan-1-one

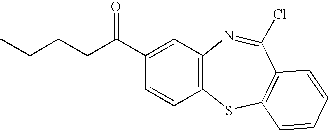

A flame dried 10 mL flask was charged under argon with 11-chloro-dibenzo[b,f][1,4]thiazepine-8-carboxylic acid methoxy-methyl-amide (34 mg, 0.10 mmol) in dry THF (2 mL) and nButyl magnesium chloride (2 M in Et₂O, 0.10 mL, 0.2 mmol) was then added. The resulting reaction mixture was stirred at room temperature for 1 hour and was then diluted with ether. The organic phase was washed with water, brine, dried (Na₂SO₃), filtered and evaporated to give crude product. Purification by flash chromatography (ethyl acetate/heptane 1:5) afforded 26.0 mg (81%) of the titled compound as a yellow oil.

¹H NMR (400 MHz, CDCl₃): δ 7.82 (1H, d, J=1.6Hz), 7.77-7.74 (2H, m), 7.53 (1H, d, J=8.4 Hz), 7.47-7.39 (3H, m), 2.90 (2H, t, J=7.2 Hz), 1.68 (2H, quintet, J=7.2 Hz), 1.37 (2H, sextet, J=7.2 Hz), 0.93 (3H, t, J=7.2 Hz). ¹³C NMR (100 MHz, CDCl₃): δ 199.5, 156.2, 146.4, 138.3, 138.1, 137.8, 133.2, 133.1(2), 132,5, 1302, 129.2, 126.6, 125.8, 38.7, 26.5,22.6, 14.1.

### Example 539

### 1-(11-cyclohexyl-dibenzo[b,f][1,4]thiazepine-8-yl)-pentan-1-one

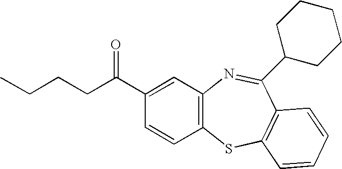

A flame dried 10 mL flask was charged under argon with the imidoyl chloride (1 eq.), Fe(acac)₃ (5 mol %) in dry THF and cooled to −40° C. Functionalized arylmagnesium halide (2 eq., 1 M in THF; prepared at −40° C.) was slowly added to the solution, keeping the temperature below −40° C. The reaction was stirred for 5 min. at −40° C., then quenched with NH₄Cl (sat., aq.) and allowed to warm to room temperature. The resulting mixture was diluted with Et₂O and the organic phase was washed with water, brine, dried (Na₂SO₃), filtered, and evaporated to give crude product. Purification by flash chromatography. The following reagents were employed: 1-(11-chloro-dibenzo[b,f][1,4]thiazepine-8-yl)-pentan-1-one (26.0 mg, 0.08 mmol), Fe(acac)₃ (1.41 mg, 0.004 mmol), THF (2 mL) and N-methylpyrrolidone (0.20 mL), cyclohexyl magnesium chloride (2 M in Et₂O, 0.08 mL, 0.16 mmol). Purification by prep. TLC (ethyl acetate/heptane 1:10) afforded 17.2 mg (57%) of the titled compound as an colorless oil.

¹H NMR (400 MHz, CDCl₃): δ 7.71 (1H, d, J=1.6 Hz), 7.59 (1H, dd, J=8.0, 2.0 Hz), 7.48-7.43 (2H, m), 7.40-7.29 (3H, m), 2.92-2.85 (3H, m), 2.21-2.17 (1H, m), 1.98-1.93 (1H, m), 1.82-1.63 (6H, m), 1.43-1.26 (6H, m), 0.92 (3H, t, J=7.2 Hz). ¹³C NMR (100 MHz, CDCl₃): δ 200.1, 177.8, 149.0, 140.1, 139.2, 137.9, 134.3, 132.6, 132.0, 130.6, 128.9, 127.4, 125.2, 124.3, 49.1, 38.6, 32.6, 30.2, 30.0, 26.6, 26.4, 26.1, 22.6, 14.1.

### Example 540

### 11-(4-fluorophenyl)-N-(thiophen-2-ylmethyl)dibenzo[b,f][1,4]thiazepine-8-carboxamide

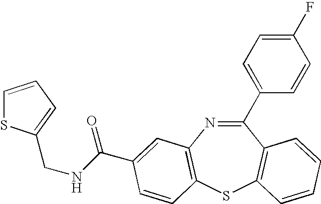

Amount isolated: 0.8 mg. LCMS m/z [M+H⁺: 444, purity (UV/MS): 100/62, t_{R}=4.97 min.

### Example 541

### 11-(5-chlorothiophen-2-yl)-N-(thiophen-2-ylmethyl)dibenzo[b,f][1,4]thiazepine-8-carboxamide

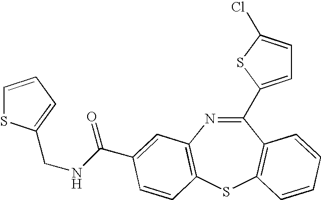

Amount isolated: 1.1 mg. LCMS m/z [M+H⁺: 466, purity (UV/MS): 99/31, t_{R}=3.00 min.

### Example 542

### 11-(3-chlorophenyl)-N-(thiophen-2-ylmethyl)dibenzo[b,f][1,4]thiazepine-8-carboxamide

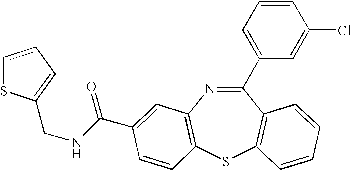

Amount isolated: 4.0 mg. LCMS m/z [M+H⁺: 460, purity (UV/MS): 99/34, t_{R}=5.35 min.

### Example 543

### 11-(4-chlorophenyl)-N-(thiophen-2-ylmethyl)dibenzo[b,f][1,4]thiazepine-8-carboxamide

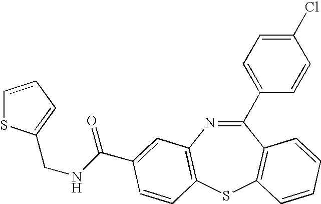

Amount isolated: 0.9 mg. LCMS m/z [M+H⁺: 460, purity (UV/MS): 100/43, t_{R}=5.35 min.

### Example 544

### 11-(3-methylthiophen-2-yl)-N-(thiophen-2-ylmethyl)dibenzo[b,f][1,4]thiazepine-8-carboxamide

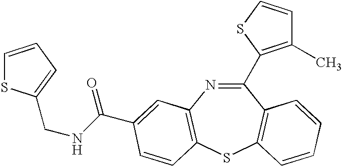

Amount isolated: 3.6 mg. LCMS m/z [M+H⁺: 446, purity (UV/MS): 100/49, t_{R}=4.93 min.

### Example 545

### 11-(3,4-dichlorophenyl)-N-(pyridin-3-ylmethyl)dibenzo[b,f][1,4thiazepine-8-carboxamide

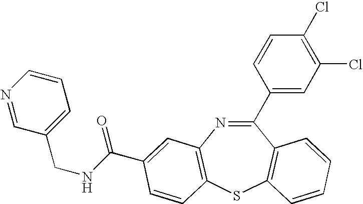

Amount isolated: 1.5 mg. LCMS m/z [M+H⁺: 489, purity (UV/MS): 96/25, t_{R}=4.93 min.

### Example 546

### 11-(4-chlorophenyl)-N-(furan-2-ylmethyl)dibenzo[b,f][1,4]thiazepine-8-carboxamide

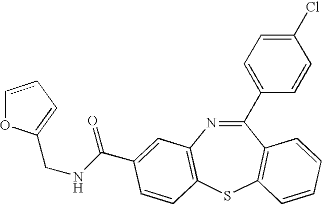

Amount isolated: 4.7 mg. LCMS m/z [M+H⁺: 444, purity (UV/MS): 100/58, t_{R}=5.19 min.

### Example 547

### 11-(4-fluorophenyl)-N-(furan-2-ylmethyl)dibenzo[b,f][1,4]thiazepine-8-carboxamide

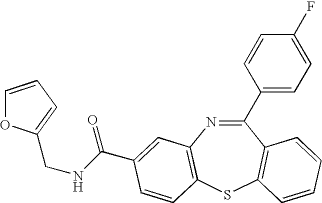

Amount isolated: 1.7 mg. LCMS m/z [M+H⁺: 428, purity (UV/MS): 99/48, t_{R}=4.73 min.

### Example 548

### 11-(5-chlorothiophen-2-yl)-N-(furan-2-ylmethyl)dibenzo[b,f][1,4]thiazepine-8-carboxamide

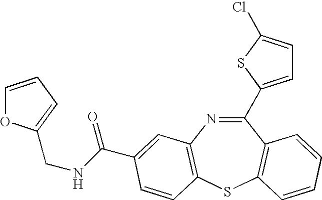

Amount isolated: 6.3 mg. LCMS m/z [M+H⁺: 450, purity (UV/MS): 100/37, t_{R}=5.21 min.

### Example 549

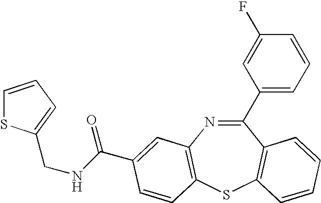

### 11-(3-fluorophenyl)-N-(thiophen-2-ylmethyl)dibenzo[b,f][1,4]thiazepine-8-carboxamide [05311 Amount isolated: 3.7 mg. LCMS m/z [M+H⁺: 444, purity (UV/MS): 100/47, t_{R}=5.07 min.

### Example 550

### 11-(3,4-dichlorophenyl)-N-(furan-2-ylmethyl)dibenzo[b,f][1,4]thiazepine-8-carboxamide

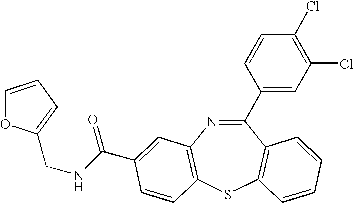

Amount isolated: 9.4 mg. LCMS m/z [M+H⁺: 478, purity (UV/MS): 100/62, t_{R}=5.55 min.

### Example 551

### 11-(3,4-dichlorophenyl)-N-(2-(pyridin-3-yl)ethyl)dibenzo[b,f][1,4]thiazepine-8-carboxami

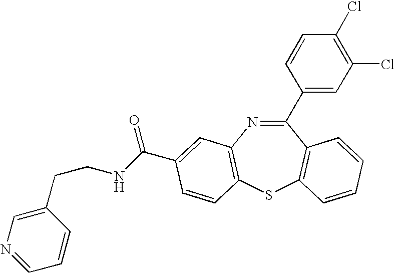

Amount isolated: 4.8 mg. LCMS m/z [M+H⁺: 503, purity (UV/MS): 100/34, t_{R}=4.93 min.

### Example 552

### 11-(3-chlorophenyl)-N-(furan-2-ylmethyl)dibenzo[b,f][1,4]thiazepine-8-carboxamid

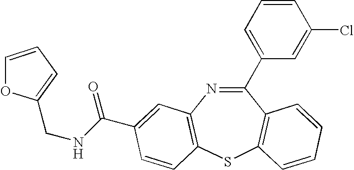

Amount isolated: 7.2 mg. LCMS m/z [M+H⁺: 444, purity (UV/MS): 100/70, t_{R}=5.13 min.

### Example 553

### 11-(5-chlorothiophen-2-yl)-N-(2-(pyridin-3-yl)ethyl)dibenzo[b,f][1,4]thiazepine-8-carboxamide

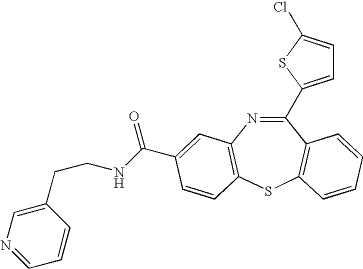

Amount isolated: 5.9 mg. LCMS m/z [M+H⁺: 475, purity (UV/MS): 97/17, t_{R}=4.52 min.

### Example 554

### 11-(3,4-dichlorophenyl)-N-(2-(pyridin-4-yl)ethyl)dibenzo[b,f][1,4]thiazepine-8-carboxamide

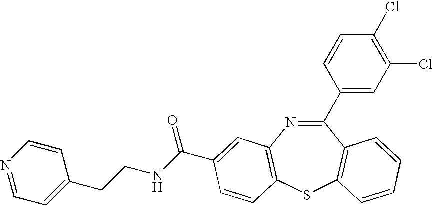

Amount isolated: 0.8 mg. LCMS m/z [M+H⁺: 504, purity (UV/MS): 97/44, t_{R}=4.90 min.

### Example 555

### 11-(5-chlorothiophen-2-yl)-N-(2-(pyridin-4-yl)ethyl)dibenzo[b,f][1,4]thiazepine-8-carboxamide

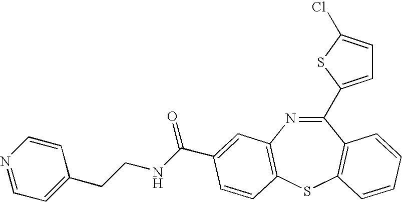

Amount isolated: 1.8 mg. LCMS m/z [M+H⁺: 475, purity (UV/MS): 100/70, t_{R}=4.52 min.

### Example 556

### 11-(4-fluorophenyl)-N-(pyridin-3-ylmethyl)dibenzo[b,f][1,4]thiazepine-8-carboxamide

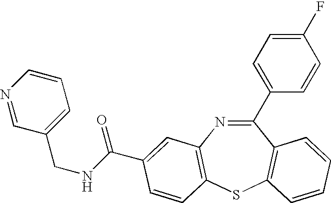

Amount isolated: 1.3 mg. LCMS m/z [M+H⁺: 439, purity (UV/MS): 99/47, t_{R}=4.05 min.

### Example 557

### 11-(4-fluorophenyl)-N-(2-(pyridin-3-yl)ethyl)dibenzo[b,f][1,4]thiazepine-8-carboxamide

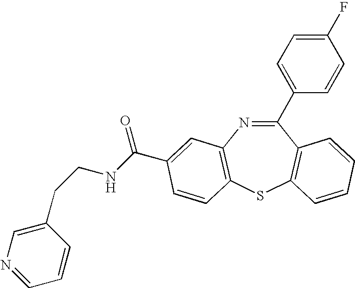

Amount isolated: 1.3 mg. LCMS m/z [M+H⁺: 453, purity (UV/MS): 98/38, t_{R}=4.07 min.

### Example 558

### 11-(3-fluorophenyl)-N-(pyridin-3-ylmethyl)dibenzo[b,f][1,4 thiazepine-8-carboxamide

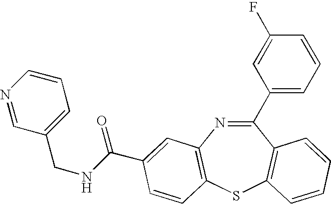

Amount isolated: 3.0 mg. LCMS m/z [M+H⁺: 439, purity (UV/MS): 99/40, t_{R}=4.10 min.

### Example 559

### 11-(4-fluorophenyl)-N-(2-(pyridin-4-yl)ethyl)dibenzo[b,f][1,4]thiazepine-8-carboxamide

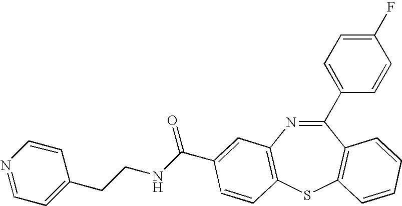

Amount isolated: 0.9 mg. LCMS m/z [M+H⁺: 453, purity (UV/MS): 89/34, t_{R}=4.07 min.

### Example 560

### 11-(2-fluorophenyl)-N-(thiophen-2-ylmethyl)dibenzo[b,f][1,4]thiazepine-8-carboxamide

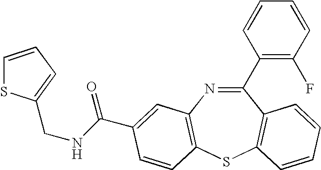

Amount isolated: 3.9 mg. LCMS m/z [M+H⁺: 444, purity (UV/MS): 100/41, t_{R}=4.67 min.

### Example 561

### 11-(3-fluorophenyl)-N-(furan-2-ylmethyl)dibenzo[b,f[]1,4]thiazepine-8-carboxamide

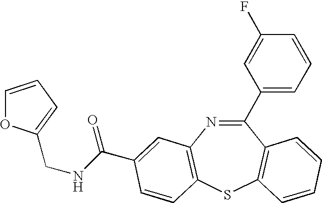

Amount isolated: 5.5 mg. LCMS m/z [M+H⁺: 428, purity (UV/MS): 100/47, t_{R}=4.75 min.

### Example 562

### N-(furan-2-ylmethyl-11-(3-methylthiophen-2-yl)dibenzo[b,f][1,4]thiazepine-8-carboxamide

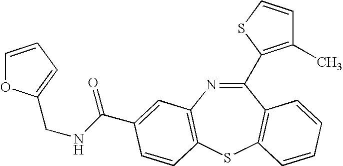

Amount isolated: 7.9 mg. LCMS m/z [M+H⁺: 430, purity (UV/MS): 95/55, t_{R}=4.70 min.

### Example 563

### 11-(3-chlorophenyl)-N-(2-(pyridin-3-yl)ethyl)dibenzo[b,f][1,4]thiazepine-8-carboxamide

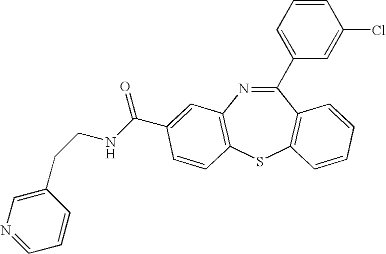

Amount isolated: 6.2 mg. LCMS m/z [M+H⁺: 469, purity (UV/MS): 100/64, t_{R}=4.50 min.

### Example 564

### 11-(2-fluorophenyl)-N-(pyridin-3-ylmethyl)dibenzo[b,f][1,4]thiazepine-8-carboxamide

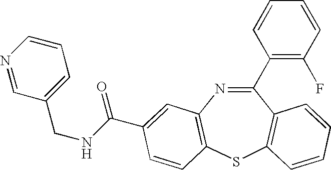

Amount isolated: 4.0 mg. LCMS m/z [M+H⁺: 439, purity (UV/MS): 99/39, t_{R}=3.75 min.

### Example 565

### 11-(4-chlorophenyl)-N-(2-(pyridin-3-yl)ethyl)dibenzo[b,f][1,4thiazepine-8-carboxamide

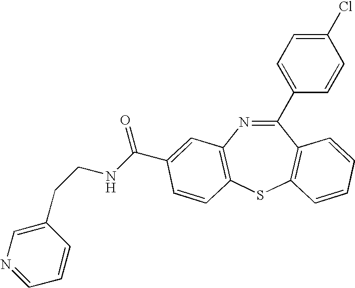

Amount isolated: 5.6 mg. LCMS m/z [M+H⁺: 469, purity (UV/MS): 88/19, t_{R}=4.50 min. Example 566

### 11-(3-fluorophenyl)-N-(2-(pyridin-3-yl)ethyl)dibenzo[b,f][1,4]thiazepine-8-carboxamide

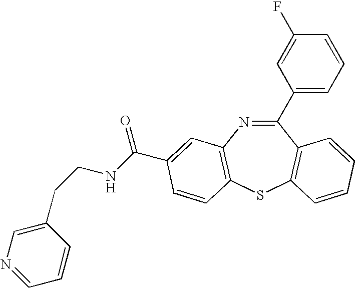

Amount isolated: 4.4 mg. LCMS m/z [M+H⁺: 453, purity (UV/MS): 100/39, t_{R}=4.12 min.

### Example 567

### 11-(4-Chlorophenyl)-N-(2-(pyridine-4-yl)ethyl)dibenzo[b,f][1,4]thiazepine-8-carboxamide

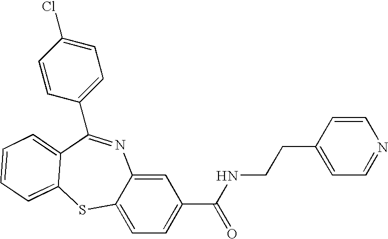

Amount isolated: 2.5 mg. LCMS m/z [M+H⁺: 469, purity (UV/MS): 97/37, t_{R}=4.47 min.

### Example 568

### 11-(pyridin-2-yl)-N-(thiophen-2-ylmethyl)dibenzo[b,f][1,4]thiazepine-8-carboxamide

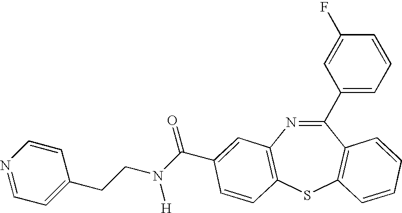

Amount isolated: 2.1 mg. LCMS m/z [M+H⁺: 427, purity (UV/MS): 100/68, t_{R}=3.88 min.

### Example 569

### 11-(3-Fluorophenyl)-N-(2-(pyridin-4-yl)ethyl)dibenzo[b,f][1,4]thiazepine-8-carboxamide

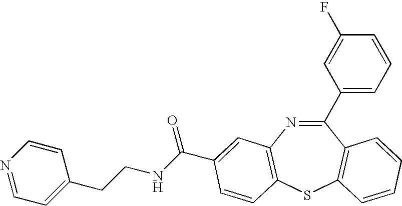

Amount isolated: 3.7 mg. LCMS m/z [M+H⁺: 453, purity (UV/MS): 94/35, t_{R}=4.08 min.

### Example 570

### 11-(2-Fluorophenyl)-N-(furan-2-ylmethyl)dibenzo[b,f][1,4]thiazepine-8-carboxamide

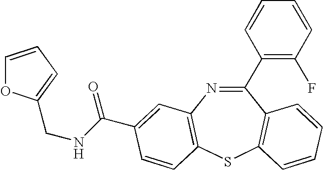

Amount isolated: 5.7 mg. LCMS m/z [M+H⁺: 428, purity (UV/MS): 94/34, t_{R}=4.45 min.

### Example 571

### 11-(3-methylthiophen-2-yl)-N-(2-(pyridin-3-yl)ethyl)dibenzo[b,f][1,4]thiazepine-8-carboxamide

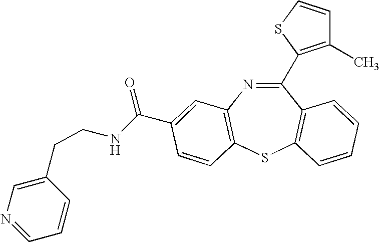

Amount isolated: 3.3 mg. LCMS m/z [M+H⁺: 455, purity (UV/MS): 100/42, t_{R}=4.02 min.

### Example 572

### 11-(2-fluorophenyl)-N-(2-(pyridin-3-yl)ethyl)dibenzo[b,f][1,4]thiazepine-8-carboxamide

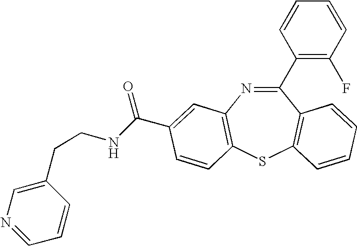

Amount isolated: 6.3 mg. LCMS m/z [M+H⁺: 453, purity (UV/MS): 95/34, t_{R}=3.78 min.

### Example 573

### N-(furan-2-ylmethyl)-11-(pyridin-2-yl)dibenzo[b,f][1,4]thiazepine-8-carboxamide

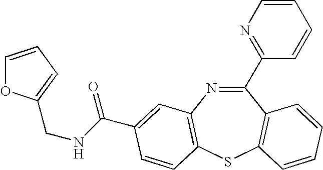

Amount isolated: 3.1 mg. LCMS m/z [M+H⁺: 411, purity (UV/MS): 100/58, t_{R}=3.62 min.

### Example 574

### 11-(3-methylthiophen-2-yl)-N-(pyridin-3-ylmethyl)dibenzo[b,f][1,4]thiazepine-8-carboxamide

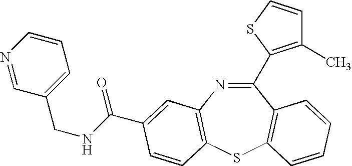

Amount isolated: 2.2 mg. LCMS m/z [M+H⁺: 441, purity (UV/MS):100/35, t_{R}=3.98 min.

### Example 575

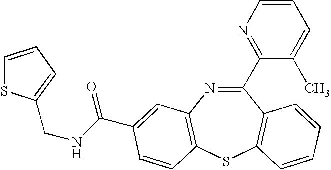

### 11-(3-methylpyridin-2-yl)-N-(thiophen-2-ylmethyl)dibenzo[b,f][1,4]thiazepine-8-carboxamide

Amount isolated: 1.6 mg. LCMS m/z [M+H⁺: 441, purity (UV/MS): 100/54, t_{R}=3.85 min.

### Example 576

### 11-(3-methylthiophen-2-yl)-N-(2-(pyridin-4-yl)ethyl)dibenzo[b,f]1,4]thiazepine-8-carboxamide

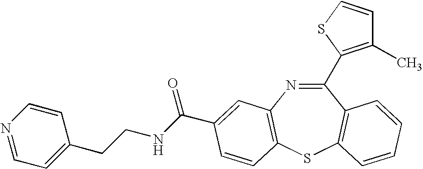

Amount isolated: 1.8 mg. LCMS m/z [M+H⁺: 455, purity (UV/MS): 98/22, t_{R}=4.00 min.

### Example 577

### 11-(3-fluorophenyl)-N-(pyridin-4-ylmethyl)dibenzo[b,f][1,4]thiazepine-8-carboxamide

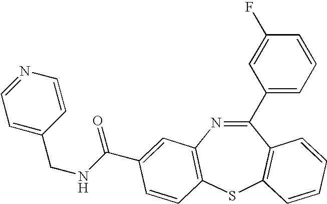

Amount isolated: 2.4 mg. LCMS m/z [M+H⁺: 439, purity (UV/MS): 98/35, t_{R}=4.03 min.

### Example 578

### 11-(2,4-dichlorophenyl)-N-(2-(pyridin-3-yl)ethyl)dibenzo[b,f][1,4]thiazepine-8-carboxamide

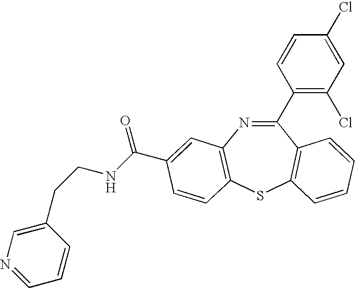

Amount isolated: 3.9 mg. LCMS m/z [M+H⁺: 503, purity (UV/MS): 96/24, t_{R}=4.58 min.

### Example 579

### 11-(2-fluorophenyl)-N-(pyridin-4-ylmethyl)dibenzo[b,f][1,4]thiazepine-8-carboxamide

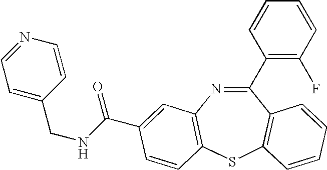

Amount isolated: 2.5 mg. LCMS m/z [M+H⁺: 439, purity (UV/MS): 100/50, t_{R}=3.72 min.

### Example 580

### 11-(2-chlorophenyl)-N-(thiophen-2-ylmethyl)dibenzo[b,f][1,4]thiazepine-8-carboxamide

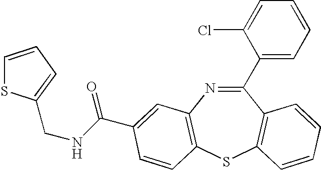

Amount isolated: 1.4 mg. LCMS m/z [M+H⁺: 460, purity (UV/MS): 99/51, t_{R}=4.88 min.

### Example 581

### 11-(2-chlorophenyl)-N-(furan-2-ylmethyl)dibenzo[b,f][1,4]thiazepine-8-carboxamide

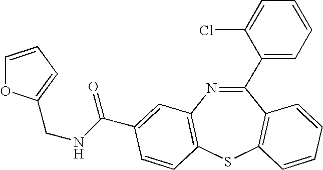

Amount isolated: 6.2 mg. LCMS m/z [M+H⁺: 444, purity (UV/MS): 100/34, t_{R}=4.65 min.

### Example 582

### 11-(3-methylthiophen-2-yl)-N-(pyridin-4-ylmethyl)dibenzo[b,f][1,4]thiazepine-8-carboxamide

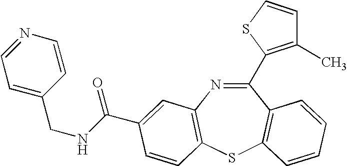

Amount isolated: 1.6 mg. LCMS m/z [M+H⁺: 441, purity (UV/MS): 99/41, t_{R}=3.97 min.

### Example 583

### 11-(2-chlorophenyl)-N-(2-(pyridin-3-yl)ethyl)dibenzo[b,f][1,4]thiazepine-8-carboxamide

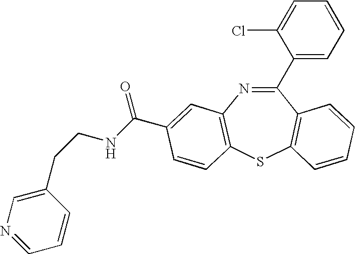

Amount isolated: 4.5 mg. LCMS m/z [M+H⁺: 470, purity (UV/MS): 100/40, t_{R}=3.97 min.

### Example 584

### 11-(pyridin-2-yl)-N-(2-(pyridin-4-yl)ethyl)dibenzo[b,f][1,4]thiazepine-8-carboxamide

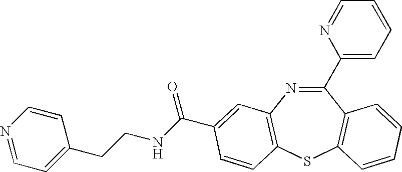

Amount isolated: 3.0 mg. LCMS m/z [M+H⁺: 436, purity (UV/MS): 98/60, t_{R}=2.90 min.

### Example 585

### 11-(2,4-dichlorophenyl)-N-(2-(pyridin-4-yl)ethyl)dibenzo[b,f][1,4thiazepine-8-carboxamide

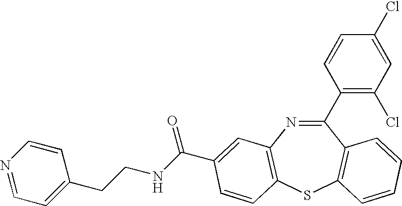

Amount isolated: 1.2 mg. LCMS m/z [M+H⁺: 503, purity (UV/MS): 96/30, t_{R}=4.58 min.

### Example 586

### 11-(2-chlorophenyl)-N-(2-(pyridin-4-yl)ethyl)dibenzo[b,f][1,4]thiazepine-8-carboxamide

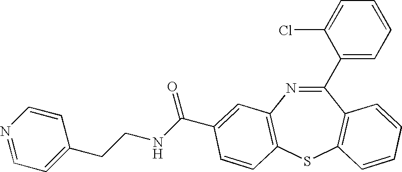

Amount isolated: 2.4 mg. LCMS m/z [M+H⁺: 469, purity (UV/MS): 93/36, t_{R}=3.95 min.

### Example 586

### 11-(3-methylpyridin-2-yl)-N-(pyridin-3-ylmethyl)dibenzo[b,f][1,4]thiazepine-8-carboxamide

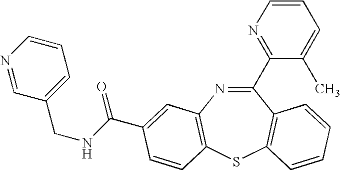

Amount isolated: 3.5 mg. LCMS m/z [M+H⁺: 436, purity (UV/MS): 92/71, t_{R}=0.93 min.

### Example 588

### 11-(3-fluorophenyl)-N-(5-methylisoxazol-3-yl)dibenzo[b,f][1,4]thiazepine-8-carboxamide

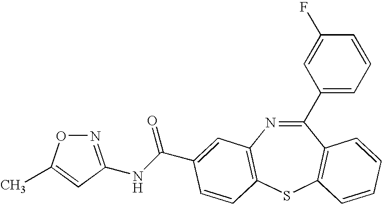

Amount isolated: 0.6 mg. LCMS m/z [M+H⁺: 430, purity (UV/MS): 100/36, t_{R}=4.92 min.

### Example 589

### 11-(3-methylpyridin-2-yl)-N-(2-(pyridin-3-yl)ethyl)dibenzo[b,f][1,4]thiazepine-8-carboxamide

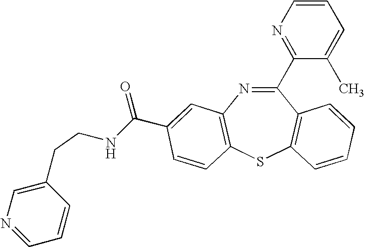

Amount isolated: 2.2 mg. LCMS m/z [M+H⁺: 450, purity (UV/MS): 93/80, t_{R}=2.93 min.

### Example 590

### 11-(4-chlorobenzylamino)-N′-(2-phenylacetyl)dibenzo[b,f][1,4thiazepine-8-carbohydrazide

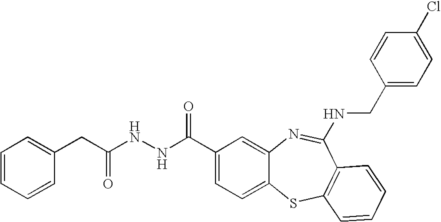

Amount isolated: 5.1 mg. LCMS m/z [M]: 527, purity (UV/MS): 97/67, t_{R}=11.92 min.

### Example 591

### N-(5-methylisoxazol-3-yl)-11-(pyridin-2-yl)dibenzo[b,f][1,4]thiazepine-8-carboxamide

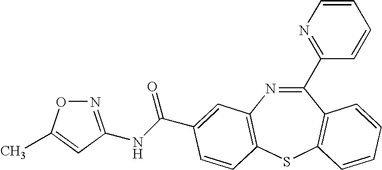

Amount isolated: 0.6 mg. LCMS m/z [M+H⁺: 412, purity (UV/MS): 97/72, t_{R}=3.68 min.

### Example 592

### 11-(2-fluorophenyl)-N-(5-methylisoxazol-3-yl)dibenzo[b,f][1,4]thiazepine-8-carboxamide

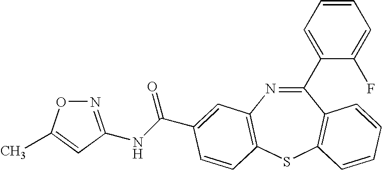

Amount isolated: 0.7 mg. LCMS m/z [M+H⁺: 429, purity (UV/MS): 100/54, t_{R}=4.59 min.

### Example 593

### 11-(4-chlorobenzylamino)-N-(pyridin-2-ylmethyl)dibenzo[b,f][1,4]thiazepine-8-carboxamide

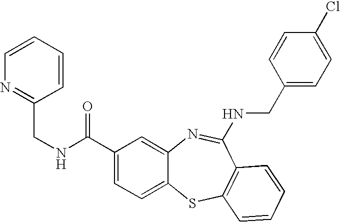

Amount isolated: 8.6 mg. LCMS m/z [M+H⁺: 485, purity (UV/MS): 95/77, t_{R}=10.02 min.

### Example 594

### N-(5-methylisoxazol-3-yl)-11-(3-methylpyridin-2-yl)dibenzo[b,f][1,4]thiazepine-8-carboxamide

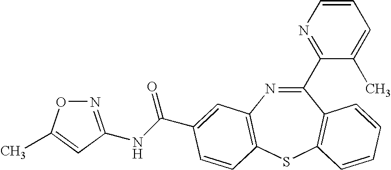

Amount isolated: 0.8 mg. LCMS m/z [M+H⁺: 426, purity (UV/MS): 99/60, t_{R}=3.72 min.

### Example 595

### 11-(4-chlorobenzylamino)-N-(2-oxoazepan-3-yl)dibenzo[b,f][1,4]thiazepine-8-carboxamide

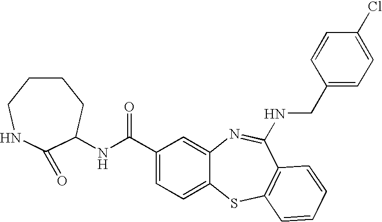

Amount isolated: 4.7 mg. LCMS m/z [M+H⁺: 505, purity (UV/MS): 88/40, t_{R}=11.6 min.

### Example 596

### N′-benzoyl-11-(4-chlorobenzylamino)dibenzo[b,f][1,4]thiazeipine-8-carbohydrazide

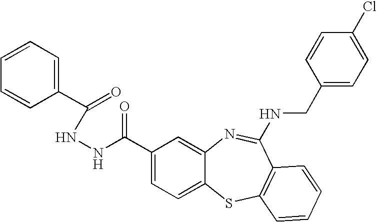

Amount isolated: 7.1 mg. LCMS m/z [M+H⁺: 513, purity (UV/MS): 97/73, t_{R}=11.66 min.

### Example 597

### 11-(3-methylpyridin-2-yl)-N-(2-(pyridin-4-yl)ethyl)dibenzo[b,f][1,4]thiazepine-8-carboxamide

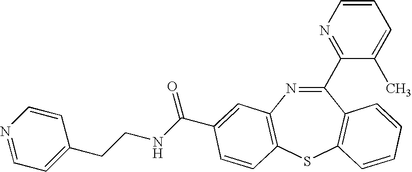

Amount isolated: 1.0 mg. LCMS m/z [M+H⁺: 450, purity (UV/MS): 88/68, t_{R}=2.92 min.

### Example 598

### 11-(4-chlorobenzylamino)-N-methoxydibenzo[b,f][1,4]thiazepine-8-carboxamide

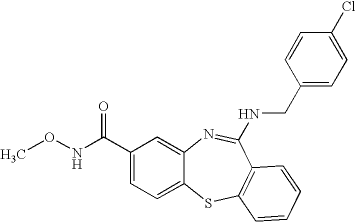

Amount isolated: 5.0 mg. LCMS m/z [M+H⁺: 424, purity (UV/MS): 92/57, t_{R}=10.82 min.

### Example 599

### 11-(2-chlorophenyl)-N-(pyridin-3-ylmethyl)dibenzo[b,f][1,4]thiazepine-8-carboxamide

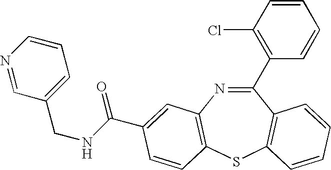

Amount isolated: 1.7 mg. LCMS m/z [M+H⁺: 455, purity (UV/MS): 99/35, t_{R}=3.95 min.

### Example 600

### N-(furan-2-ylmethyl 11-(3-methylpyridin-2-yl)dibenzo[b,f][1,4]thiazepine-8-carboxamide

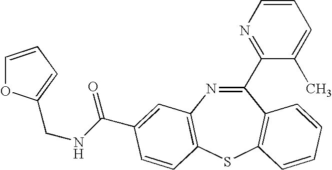

Amount isolated: 2.1 mg. LCMS m/z [M+H⁺: 425, purity (UV/MS): 86/40, t_{R}=3.65 min.

### Example 601

### 11-(pyridin-2-yl)-N-(2-(pyridin-3-yl)ethyl)dibenzo[b,f][1,4thiazelpine-8-carboxamide

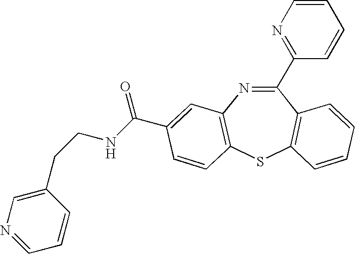

Amount isolated: 3.6 mg. LCMS m/z [M+H⁺: 436, purity (UV/MS): 96/61, t_{R}=2.90 min.

### Example 602

### 11-(4-chlorobenzylamino)-N-(pyridin-3-ylmethyl)dibenzo[b,f][1,4]thiazepine-8-carboxamide

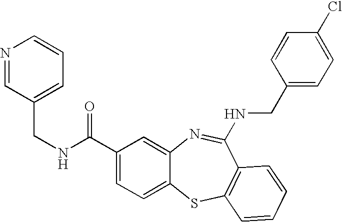

Amount isolated: 8.9 mg. LCMS m/z [M+H⁺: 485, purity (UV/MS): 99/100, t_{R}=9.877 min.

### Example 603

### 11-(2-chlorophenyl)-N-(pyridin-4-ylmethyl)dibenzo[b,f][1,4]thiazepine-8-carboxamide

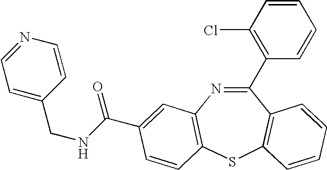

Amount isolated: 1.8 mg. LCMS m/z [M+H⁺: 455, purity (UV/MS): 97/39, t_{R}=3.92 min.

### Example 604

### 11-(4-chlorobenzylamino)-N-(pyridin-4-ylmethyl)dibenzo[b,f][1,4]thiazepine-8-carboxamide

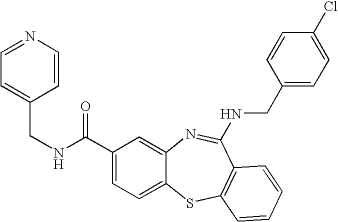

Amount isolated: 4.7 mg. LCMS m/z [M+H⁺: 485, purity (UV/MS): 93/95, t_{R}=9.88 min.

### Example 605

### 11-(4-chlorobenzalamino)-N-(4-sulfamoylbenzyl)dibenzo[b,f][1,4]thiazepine-8-carboxamide

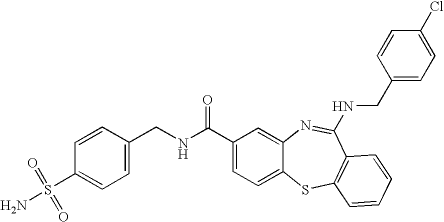

Amount isolated: 6.1 mg. LCMS m/z [M+H⁺: 563, purity (UV/MS): 77/43, t_{R}=11.56 min.

### Example 606

### 11-(5-Bromopyridin-2-yl)-dibenzo[b,f][1,4]thiazepine-8-carboxylic acid butylamide,

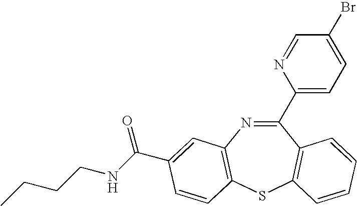 Preparation of the Zinc Reagent:

A dry flask equipped with a magnetic bar was charged with zinc dust. The reaction flask was flushed with argon and a solution of 1,2-dibromoethane (100 mg, 0.53 mmol) in N,N-dimethylacetamide (1.5 mL) was added. The zinc suspension was shortly heated with a heat gun until evolution of ethylene occurred (repeated twice).

The reaction mixture was allowed to cool to room temperature. Trimethylsilyl chloride (0.30 mL, 2.3 mmol) was added in two portions. After 15 minutes stirring at room temperature a solution of 5-bromo-2-iodopyridine (1.42 g, 5.0 mmol) in N,N-dimethylacetamide (3.0 mL) was added to the zinc suspension at 50° C. The reaction mixture was stirred at 70° C. for 3 hours. Conversion of the starting material was followed by GC using decane as the internal standard. After 3 hours at 70° C., 60% of the starting material was converted to the desired zinc reagent. Stirring was continued overnight at 70° C., which gave full conversion. The reaction mixture was allowed to cool to room temperature and diluted with dry THF (3.0 mL). The remaining zinc was allowed to settle. The obtained solution of 5-bromo-2-pyridylzinc iodide was used immediately in the next step.

Bis(dibenzylideneacetone)palladium (18 mg, 0.031 mmol) and tri-2-furylphosphine (15 mg, 0.065 mmol) were dissolved in dry THF (1.0 mL) in a dry flask, under argon atmosphere. A solution of 11-chloro-dibenzo[b,f][1,4]thiazepine-carboxylic acid butylamide (prepared as previously described, 200 mg, 0.63 mmol) in dry THF (2.0 mL) was added to the flask. A solution of the freshly prepared 5-bromo-2-pyridylzinc iodide (3 mL, 2.0 mmol) was added dropwise to the reaction mixture at room temperature. After 20 hours stirring at room temperature the reaction mixture was partitioned between aqueous NH₄Cl (sat) and EtOAc. The organic layer was dried over Na₂SO₄, filtered end evaporated to dryness. The residue was purified by silica gel column chromatography, eluting with a stepwise gradient of 15-30% EtOAc in toluene. The isolated product was repurified using an acidic-exchange cartridge eluting with NH₃ in MeOH. Yield: 5.8 mg, 2%.

LCMS m/z 467 [M+H]⁺ HPLC t_{R}=4.3 min. ¹H NMR (CDCl₃, 400 MHz) δ 8.72 (m, 1H, Ar—H), 8.24 (m, 1H, Ar—H), 8.01-7.98 (m, 1H, Ar—H), 7.71 (m, 1H, Ar—H), 7.66-7.52 (m, 3H, Ar—H), 7.43 (m, 1H, Ar—H), 7.32 (m, 1H, Ar—H), 7.21 (m, 1H, Ar—H), 6.12-6.03 (broad s, 1H, NH), 3.45 (q, 2H, J=7.2 Hz, CH_{2Bu}), 1.58 (pentet, 2H, J=7.2 Hz, CH_{2Bu}), 2.80 (m, 2H, J=7.2 Hz, CH_{2Bu}), 0.95 (t, 3H, J=7.2 Hz, CH_{3Bu}).

### Example 607

### General Procedure for the Synthesis of the Zinc Reagents from bromopyridines

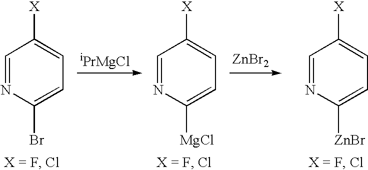

2-Bromo-5-halopyridine (3 mmol) was dissolved in THF (5.5 mL) and isopropylmagnesium chloride (2 M in THF; 1.5 mL; 3.0 mmol) was added at room temperature. After 2hours, zinc bromide (1 M in THF; 3.0 mL; 3.0 mmol) was added and the mixture was stirred at room temperature under argon over night. The crude mixture was used immediately in the next step.

### Example 608

### 11-(5-Fluoropyridin-2-yl)-dibenzo[b,f][1,4]thiazepine-carboxylic acid butylamide

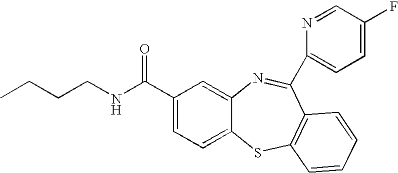

A reaction flask was charged with 11-chloro-dibenzo[b,f][1,4]thiazepine-carboxylic acid butylamide (0.17 g; 0.50 mmol) and bis(triphenylphosphine)palladium(II) chloride (36.0 mg; 0.050 mmol) under argon. THF (5 mL) was added followed by the addition of 5-fluoro-2-pyridylzinc bromide (0.15 M in THF; 12.5 mL; 1.8 mmol) at room temperature. After 5 hours, aqueous NH₄Cl (sat) was added to the mixture and extracted with EtOAc. The combined organic layers were washed with brine, dried (Na₂SO₄), filtered and concentrated in vacuo. The crude mixture was purified by silica gel column chromatography (0-20% EtOAc in toluene) followed by ion exchange column chromatography (eluting with 2% NH₃ in MeOH) and recrystallization from MeOH to yield the title compound as a yellow solid (54.4 mg; 27%).

LCMS m/z 406 [M+H]⁺, purity (UV/MS) 99/95, t_{R}=8.38 min. ¹H NMR (CDCl₃, 400 MHz) δ 8.50 (d, 1H, J=2.8 Hz, ArH), 8.38-8.42 (m, 1H, ArH), 7.68 (d, 1H, J=0.4 Hz, ArH), 7.50-7.58 (m, 4H, ArH), 7.40-7.44 (m, 1H, ArH), 7.30-7.34 (m, 1H, ArH), 7.20-7.25 (m, 1H, ArH), 6.03 (br m, 1H, NH), 3.44 (q, 2H, J=6.8 Hz, CH₂), 1.54-1.62 (m, 2H, CH₂), 1.36-1.45 (m, 2H, CH₂), 0.95 (t, 3H, J=7.2 Hz, CH₃).

### Example 609

### 11-(5-Chloropyridin-2-yl)-dibenzo[b,f][1,4]thiazepine-carboxylic acid butylamide

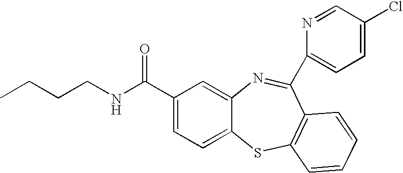

A reaction flask was charged with 11-chloro-dibenzo[b,f][1,4]thiazepine-carboxylic acid butylamide (0.17 g; 0.50 mmol) and bis(triphenylphosphine)palladium(II) chloride (36.0 mg; 0.050 mmol) under argon. THF (5 mL) was added followed by the addition of 5-chloro-2-pyridylzinc bromide (0.15 M in THF; 12.5 mL; 1.8 mmol) at room temperature. After 5 hours, aqueous NH₄Cl (sat) was added to the mixture and extracted with EtOAc. The combined organic layers were washed with brine, dried (Na₂SO₄), filtered and concentrated in vacuo. The crude mixture was purified by silica gel column chromatography (0-20% EtOAc in toluene) followed by ion exchange column chromatography (eluting with 2% NH₃ in MeOH) and recrystallization from MeOH to yield the title compound as a yellow solid (69.5 mg; 33%).

LCMS m/z 422 [M+H]⁺, purity (UV/MS) 98/88, t_{R}=6.43 min. ¹H NMR (CDCl₃, 400 MHz) δ 8.60 (d, 1H, J=1.6 Hz, ArH), 8.31 (d, 1H, J=8.8 Hz, ArH), 7.82-7.85 (m, 1H, ArH), 7.69 (d, 1H, J=0.4 Hz, ArH), 7.51-7.55 (m, 3H, ArH), 7.40-7.44 (m, 1H, ArH), 7.32-7.34 (m, 1H, ArH), 7.20-7.25 (m, 1H, ArH), 6.03 (br m, 1H, NH), 3.44 (q, 2H, J=7.2 Hz, CH₂), 1.54-1.62 (m, 2H, CH₂), 1.37-1.47 (m, 2H, CH₂), 0.95 (t, 3H, J=7.6 Hz, CH₃).

### Example 610

### 11-(5-Fluoropyridin-2-yl)-dibenzo[b,f][1,4]thiazepine-carboxylic acid piperidin-1-ylamide

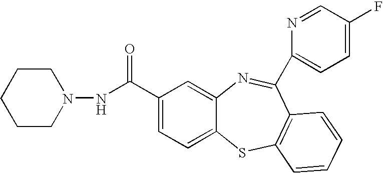

A reaction flask was charged with 11-chloro-dibenzo[b,f][1,4]thiazepine-carboxylic acid piperidin-1-ylamide (80.0 mg; 0.22 mmol) and bis(triphenylphosphine) palladium(II)chloride (15.1 mg; 0.022 mmol) under argon. THF (3 mL) was added followed by the addition of 5-fluoro-2-pyridylzinc bromide (0.15 M in THF; 5.0 mL; 0.75 mmol) at room temperature. After 3 hours, aqueous NH₄Cl (sat) was added to the mixture and extracted with EtOAc. The combined organic layers were washed with brine, dried (Na₂SO₄), filtered and concentrated in vacuo. The crude mixture was purified by silica gel column chromatography (0-30% EtOAc in toluene), ion exchange column chromatography (eluting with 2% NH₃ in MeOH) and recrystallization from EtOAc to yield the title compound as a yellow solid (9.8 mg; 10%).

LCMS m/z 433 [M+H]⁺, purity (UV/MS) 97/92, t_{R}=3.80 min. ¹H NMR (CDCl₃, 400 MHz) δ 8.50 (d, 1H, J=2.8 Hz, ArH), 8.38-8.40 (m, 1H, ArH), 7.67 (d, 1H, J=0.4 Hz, ArH), 7.51-7.56 (m, 4H, ArH), 7.40-7.44 (m, 1H, ArH), 7.30-7.34 (m, 1H, ArH), 7.20-7.24 (m, 1H, ArH), 6.69 (br m, 1H, NH), 2.82-2.86 (m, 4H, CH₂), 1.73-1.79 (m, 4H, CH₂), 1.44-1.48 (m, 2H, CH₂).

### Example 611

### 11-(5-Chloropyridin-2-yl)-dibenzo[b,f][1,4]thiazepine-carboxylic acid piperidin-1-ylamide

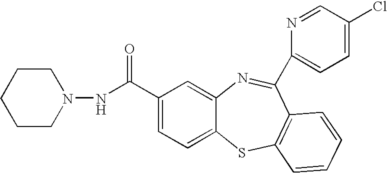

A reaction flask was charged with 11-chloro-dibenzo[b,f][1,4]thiazepine-carboxylic acid piperidin-1-ylamide (80.0 mg; 0.22 mmol) and bis(triphenylphosphine) palladium(II)chloride (15.1 mg; 0.022 mmol) under argon. THF (3 mL) was added followed by the addition of 5-chloro-2-pyridylzinc bromide (0.15 M in THF; 5.0 mL; 0.75 mmol) at room temperature. After 3 hours, aqueous NH₄Cl (sat) was added to the mixture and extracted with EtOAc. The combined organic layers were washed with brine, dried (Na₂SO₄), filtered and concentrated in vacuo. The crude mixture was purified by silica gel column chromatography (0-30% EtOAc in toluene) and recrystallization from EtOAc to yield the title compound as a yellow solid (13.8 mg; 14%).

LCMS m/z 449 [M+H]⁺, purity (UV/MS) 99/87, t_{R}=7.94 min. ¹H NMR (CDCl₃, 400 MHz) δ 8.60 (d, 1H, J=1.6 Hz, ArH), 8.301 (d, 1H, J=8.0 Hz, ArH), 7.82-7.84 (m, 1H, ArH), 7.67 (d, 1H, J=0.4 Hz, ArH), 7.51-7.55 (m, 3H, ArH), 7.40-7.44 (m, 1H, ArH), 7.30-7.34 (m, 1H, ArH), 7.20-7.22 (m, 1H, ArH), 6.68 (br m, 1H, NH), 2.81-2.83 (m, 4H, CH₂), 1.74-1.78 (m, 4H, CH₂), 1.42-1.48 (m, 2H, CH₂).

### Example 612

### Receptor Selection and Amplification Technology Assay

The functional receptor assay, Receptor Selection and Amplification Technology (R-SAT®), was used to investigate the pharmacological properties of known and novel CBI compounds. R-SAT is disclosed in U.S. Pat. Nos. 5,707,798, 5,912,132, and 5,955,281, all of which are hereby incorporated herein by reference in their entirety, including any drawings.

Briefly, NIH3T3 cells were grown in 96 well tissue culture plates to 70-80% confluence. Cells were transfected for 16-20 h with plasmid DNAs using Polyfect (Qiagen Inc.) using the manufacturer's protocols. R-SATs were generally performed with 10 ng/well of receptor, 10 ng/well of Gqi5 (Conklin et al, Nature 1993 363:274-6) and 20 ng/well of β-galactosidase plasmid DNA. All receptor constructs used were in the pSI-derived mammalian expression vector (Promega Inc). The CB1 receptor gene was amplified by PCR from genomic DNA using oligodeoxynucleotide primers based on the published sequence (GenBank Accession # X54937) SEQ ID NO: 1 encodes a CB1 receptor truncated after amino acid 417 (SEQ ID NO: 2). The CB2 gene was cloned by performing a PCR reaction on mRNA from spleen. The PCR product containing the entire coding sequence of the CB2 gene was cloned into an expression vector such that the CB2 gene was operably linked to an SV40 promoter. The sequence of the CB2 gene (GenBank Accession #NM_{—}001841) is provided as SEQ ID NO: 3 and the sequence of the encoded CB2 polypeptide is provided as SEQ ID NO: 4. For large-scale transfections, cells were transfected for 16-20 h, then trypsinized and frozen in DMSO. Frozen cells were later thawed, plated at ˜10,000 cells per well of a 96 half-area well plate that contained drug. With both methods, cells were then grown in a humidified atmosphere with 5% ambient CO₂ for five days. Media was then removed from the plates and marker gene activity was measured by the addition of the β-galactosidase substrate o-nitrophenyl β-D-galactopyranoside (ONPG) in PBS with 0.5% NP-40. The resulting colorimetric reaction was measured using a spectrophotometric plate reader (Titertek Inc.) at 420 nm. All data was analyzed using the XLFit (IDBSm) computer program. pIC₅₀ represents the negative logarithm of the concentration of ligand that caused 50% inhibition of the constitutive receptor response. Percent inhibition was calculated as the difference between the absorbance measurements in the absence of added ligand compared with that in the presence of saturating concentrations of ligand normalized to the absorbance difference for the reference ligand (SR141716), which was assigned a value of 100%.

These experiments provide a molecular profile, or fingerprint, for each of these agents at the human CB1 receptor. As can be seen in Table 1, the compounds are inverse agonists at the CB1 receptor. Additional pIC₅₀ data shown in Appendix A.

TABLE 1

| | |
|---|---|
| CB1 (mutant) | CB1 (wild-type) |

| Compound | pIC₅₀ | % Inhibition | pIC₅₀ | % Inhibition |
|---|---|---|---|---|
| 1 | 6.8 | 80 | 7.4 | 67 |
| 9 | 7.4 | 105 | 7.9 | 99 |
| 11 | 6.9 | 95 | 7.8 | 84 |
| 18 | 6.7 | 99 | 7.4 | 99 |
| 14 | 6.5 | 94 | 6.9 | 88 |
| 100 | 7.6 | 127 | | |
| 101 | 7.2 | 87 | | |
| 102 | 5.7 | 92 | | |
| 103 | 8.6 | 98 | | |
| 104 | 8.0 | 107 | | |
| 105 | 7.0 | 83 | | |

% Inhibition is relative to the ligand SR141716.

It will be appreciated that the foregoing assay may be used to identify compounds which are agonists, inverse agonists or antogonists of a cannabinoid receptor. In some embodiments, the cannabinoid receptor used in the assay may be a CB1 receptor. In other embodiments, the cannabinoid receptor used in the assay may consist essentially of SEQ ID NO: 2. In further embodiments, the cannabinoid receptor used in the assay may have at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99% or greater than at least 99% amino acid identity with a full-length CB1 receptor or a truncated CB1 receptor of SEQ ID NO: 2.

Using the following methods, the compounds disclosed herein were evaluated for their ability to bind to a CB1 receptor. The compounds were tested using a receptor binding assay and then determining of any change in GTPgamma S binding of transfected cells.

### Example 613

### CB1 Receptor Binding Assays

To show that CB1 antagonists can block binding of selective CB1 ligands to native CB1 receptors the ability of compounds of Formula I to block binding of the highly CB1-selective ligand SR1411716 was examined in rat brain membrane preparations as follows.

Membrane preparations—Whole brains were harvested from Harlan Sprague Dawley rats and placed in 50 ml Falcon Tubes on ice. The volume was made up to 30 ml with ice-cold membrane buffer (20 mM HEPES, 6 mM MgCl₂, 1 mM EDTA, pH 7.2). The Brains were homogenized with a Brinkmann Polytron PT3000 at 20,000 rpm for 40 s. The homogenate was spun at 1,000×g for 10 min at 4° C. to remove nuclei and cellular debris. The supernatant was collected and re-centrifuged as previously before membranes were precipitated at 45,000×g for 20 min at 4° C., resuspended in membrane buffer to a final concentration of 1 mg/ml, snap frozen as aliquots in liquid nitrogen and stored at −80° C.

Membrane Binding—10 μg of membranes were incubated in binding buffer (1×DMEM with 0.1%BSA) in the presence of 3 nM radioligand ([³H]SR141716A, Amersham Biosciences, Piscataway, N.J.) and varying concentrations of ligands (total volume 100 μl in a 96 well plate). Cells were filtered onto a 96 well GF/B filterplate (Packard Bioscience, Shelton Conn.) and washed with 300 ml wash buffer (25mM HEPES, 1. mM CaCl₂, 5 mM MgCl₂0.25M NaCL) using a Filtermate 196 Harvester (Packard Instruments, Downers Grove, Ill.). The filter plates were dried under a heat lamp before addition of 50 μl of scintillation fluid to each well (Microsoft 20, Packard, Shelton, Conn.). Plates were counted on a Topcount NXT (Packard, Shelton, Conn.).

Data analysis—Graphs were plotted and K_{D} values were determined by nonlinear regresson analysis using Prism software (GraphPad version 4.0, San Diego, Calif., USA).

TABLE 2
Binding of CB1 antagonists to native CB1 receptors

| Compound ID | Rat Brain pKi |
|---|---|
| SR 141716 | 9.1 |
| 2 | 8.2 |
| 5 | 6.7 |
| 9 | 8.3 |
| 11 | 8.0 |
| 12 | 6.6 |
| 19 | 7.3 |
| 100 | 7.1 |

It will be appreciated that the CB1 receptor binding assay of the foregoing example may be used to identify compounds which are agonists, inverse agonists or antogonists of a cannabinoid receptor. In some embodiments, the cannabinoid receptor used in the assay may be a CB1 receptor. In other embodiments, the cannabinoid receptor used in the assay may consist essentially of SEQ ID NO: 2. In further embodiments, the cannabinoid receptor used in the assay may have at least 30%, at least 35%, at least 40%, at least 45%, at least 50% , at least 55%, at least 60% , at least 65%, at least 70%, at least 75%, at least 80%, at least 85%, at least 90%, at least 95%, at least 96%, at least 97%, at least 98%, at least 99% or greater than at least 99% amino acid identity with a full-length CB1 receptor or a truncated CB1 receptor of SEQ ID NO: 2.

### Example 614

### Sequences for Truncated CB1 Receptors

Below are sequences encoding a truncated CB1 receptor.

SEQ ID NO: 1:
ATGAAGTCGATCCTAGATGGCCTTGCAGATACCACCTTCCGCACCATCACCACTG
ACCTCCTGTACGTGGGCTCAAATGACATTCAGTACGAAGACATCAAAGGTGACA
TGGCATCCAAATTAGGGTACTTCCCACAGAAATTCCCTTTAACTTCCTTTAGGGG
AAGTCCCTTCCAAGAGAAGATGACTGCGGGAGACAACCCCCAGCTAGTCCCAGC
AGACCAGGTGAACATTACAGAATTTTACAACAAGTCTCTCTCGTCCTTCAAGGAG
AATGAGGAGAACATCCAGTGTGGGGAGAACTTCATGGACATAGAGTGTTTCATG
GTCCTGAACCCCAGCCAGCAGCTGGCCATTGCAGTCCTGTCCCTCACGCTGGGCA
CCTTCACGGTCCTGGAGAACCTCCTGGTGCTGTGCGTCATCCTCCACTCCCGCAG
CCTCCGCTGCAGGCCTTCCTACCACTTCATCGGCAGCCTGGCGGTGGCAGACCTC
CTGGGGAGTGTCATTTTTGTCTACAGCTTCATTGACTTCCACGTGTTCCACCGCAA
AGATAGCCGCAACGTGTTTCTGTTCAAACTGGGTGGGGTCACGGCCTCCTTCACT
GCCTCCGTGGGCAGCCTGTTCCTCACAGCCATCGACAGGTACATATCCATTCACA
GGCCCCTGGCCTATAAGAGGATTGTCACCAGGCCCAAGGCCGTGGTGGCGTTTT
GCCTGATGTGGACCATAGCCATTGTGATCGCCGTGCTGCCTCTCCTGGGCTGGAA
CTGCGAGAAACTGCAATCTGTTTGCTCAGACATTTTCCCACACATTGATGAAACC
TACCTGATGTTCTGGATCGGGGTCACCAGCGTACTGCTTCTGTTCATCGTGTATG
CGTACATGTATATTCTCTGGAAGGCTCACAGCCACGCCGTCCGCATGATTCAGCG
TGGCACCCAGAAGAGCATCATCATCCACACGTCTGAGGATGGGAAGGTACAGGT
GACCCGGCCAGACCAAGCCCGCATGGACATTAGGTTAGCCAAGACCCTGGTCCT
GATCCTGGTGGTGTTGATCATCTGCTGGGGCCCTCTGCTTGCAATCATGGTGTAT
GATGTCTTTGGGAAGATGAACAAGCTCATTAAGACGGTGTTTGCATTCTGCAGTA
TGCTCTGCCTGCTGAACTCCACCGTGAACCCCATCATCTATGCTCTGAGGAGTAA
GGACCTGCGACACGCTTTCCGGAGCATGTTTCCCTCTTGTGAAGGCTAG
SEQ ID NO: 2
MKSILDGLADTTFRTITTDLLYVGSNDIQYEDIKGDMASKLGYFPQKFPLTSFRGSPFQ
EKMTAGDNPQLVPADQVNITEFYNKSLSSFKENEENIQCGENFMDIECFMVLNPSQQ
LAIAVLSLTLGTFTVLENLLVLCVILHSRSLRCRPSYHFIGSLAVADLLGSVIFVYSFIDF
HVFHRKDSRNVFLFKLGGVTASFTASVGSLFLTAIDRYISIHRPLAYKRIVTRPKAVVA
FCLMWTIAIVIAVLPLLGWNCEKLQSVCSDIFPHIDETYLMFWIGVTSVLLLFIVYAYM
YILWKAHSHAVRMIQRGTQKSIIIHTSEDGKVQVTRPDQARMDIRLAKTLVLILVVLII
CWGPLLAIMVYDVFGKMNKLIKTVFAFCSMLCLLNSTVNPIIYALRSKDLRHAFRSM
FPSCEG*

### Example 615

### Acute Feeding Study

Male, Sprague-Dawley rats (90-120 g) served as subjects for these studies. Rats were fasted for a period of 16 hrs (water was always available). After the fasting period, test compounds were administered either intraperitoneally (ip) or orally (po). Immediately following compound administration, the rats were returned to their home cage. Following 30 min after compound administration, the rats were removed from their home cages and placed individually into clean cages with a pre-measured amount of food. Food weights were obtained (to the nearest 0.1 g) at various time points. Food consumption was monitored for a period of up to 2 hrs (i.e., 2.5 hr after test compound administration).

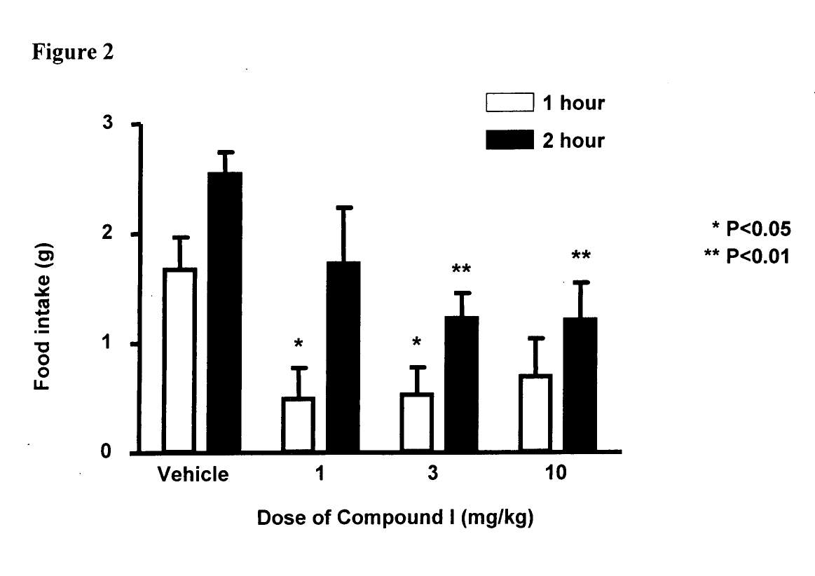

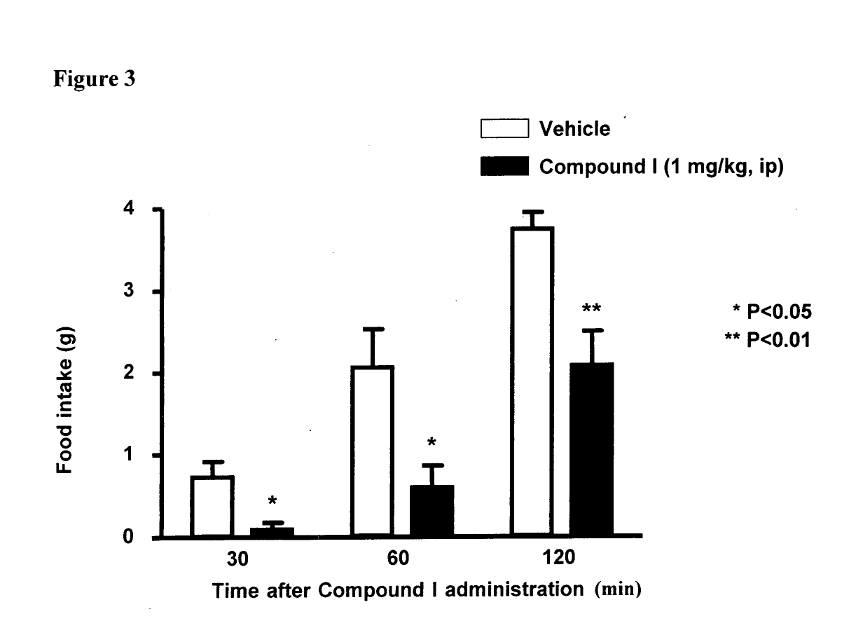

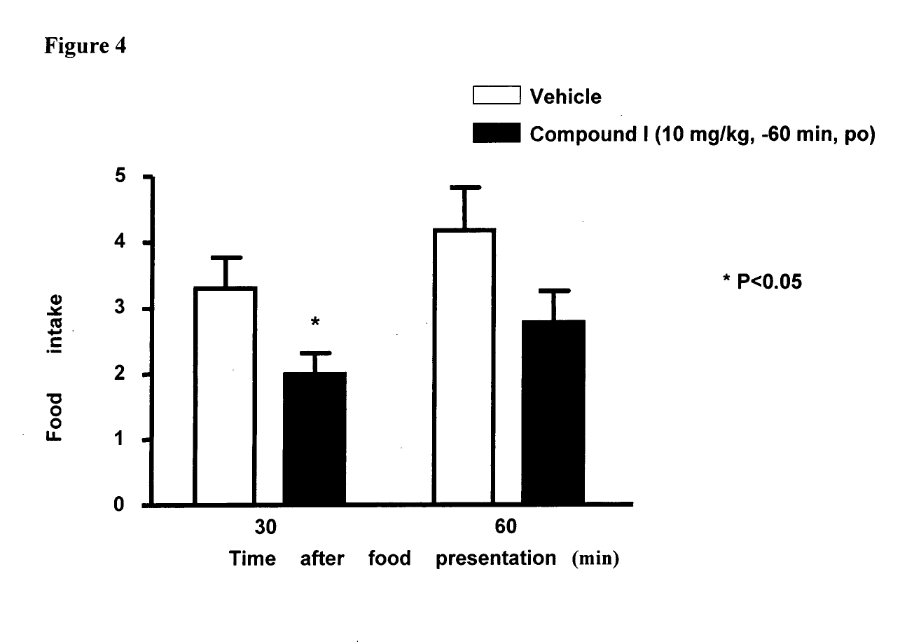

FIG. 2 is a bar graph showing the food intake in fasted rats 1 and 2 hours after being administered either 1, 3, or 10 mg/kg doses of Compound I. * Indicates p<0.05 as compared to the vehicle-treated controls. ** Indicates p<0.01 as compared to the vehicle-treated controls. FIG. 3 is bar graph showing the time course food intake in fasted rats after being administered 1 mg/kg of Compound I. * Indicates p<0.05 as compared to the vehicle-treated controls. ** Indicates p<0.01 as compared to the vehicle-treated controls. FIG. 4 is a bar graph showing cumulative food consumption at several points in time after the rats had been dosed with 10 mg/kg of Compound I. * Indicates p<0.05 as compared to the vehicle-treated controls. As shown by FIGS. 2-4, Compound I suppresses the cumulative food intake in fasted rats. FIG. 2 also shows that suppression of food take is dose-dependent.

### Example 616

### Tail Flick Study

Male, NSA mice (15-20 g) served as subjects for these studies. Baseline nociceptive thresholds were assessed using the warm water tail flick test. Briefly, the distal ⅓ to ½ of the tail was immersed in a 52° C. water bath and the time (to the nearest 0.1 sec) until the mouse removed its tail (i.e., “flicks”) from the water was recorded (i.e., tail flick latency). Mice were then injected ip with either vehicle or with various doses of the CB1 agonist CP 55,940 and tail flick latencies were recorded for a period of up to 3 hr. A maximum latency of 10 sec was employed in order to prevent tissue damage. In order to determine if a CB1 inverse agonists could block the antinociceptive actions of CP 55,940, mice were pretreated with either vehicle or with a test compound 30 min prior to CP55,940. CP55,940 (1 mg/kg) was administered subcutaneously, and Compound I was administered intraperitoneally. Tail flick latencies were then obtained at various time points for a period of up to 2 hr. The vehicle for both compounds was 1:1:18 cremphor:ethanol:saline.

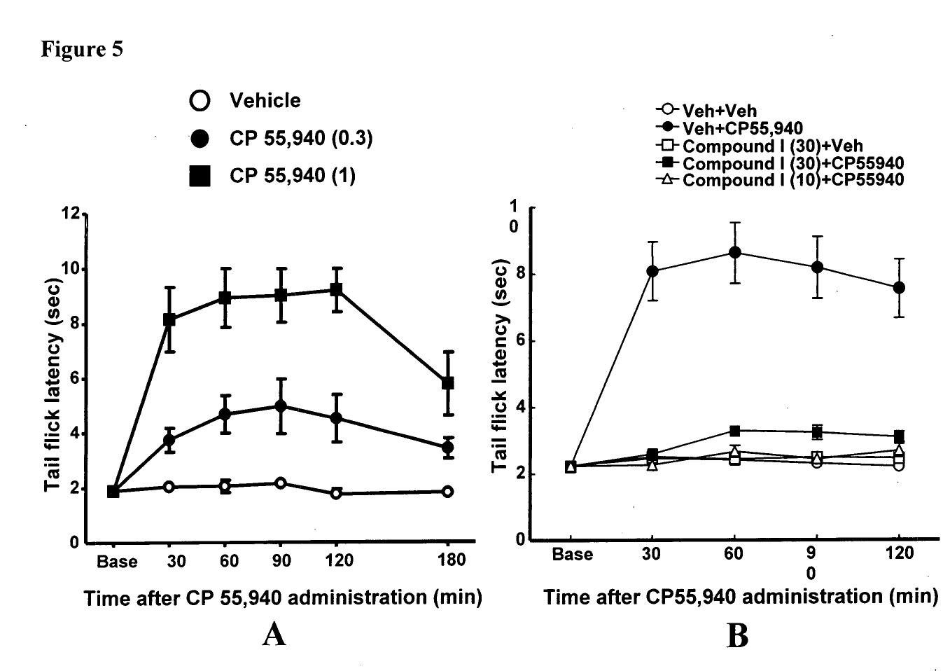

FIG. 5A is a line graph showing the attenuation of CB1 agonist-mediated effects after administration of CP 55,940 (0.3 and 1.0 mg/kg). FIG. 5B is a line graph showing the attenuation of CB1 agonist-mediated effects after administration of Compound I alone or in combination with CP55,940. As indicated by FIGS. 5A and 5B, Compound I attenuates the antinociceptive actions of CP55,940.

### Example 617

### Hypothermia Study

Male, NSA mice (15-20 g) served as subjects for these studies. In order to determine if the test compound could block hypothermia elicited by CP 55,940 (1 mg/kg, ip), mice were pretreated with either vehicle or with test compound 30 min prior to CP55,940. Core body temperatures were then obtained at various time points following CP 55,940 administration. Core body temperature (to the nearest 0.1° C.) was obtained by rectal probe.

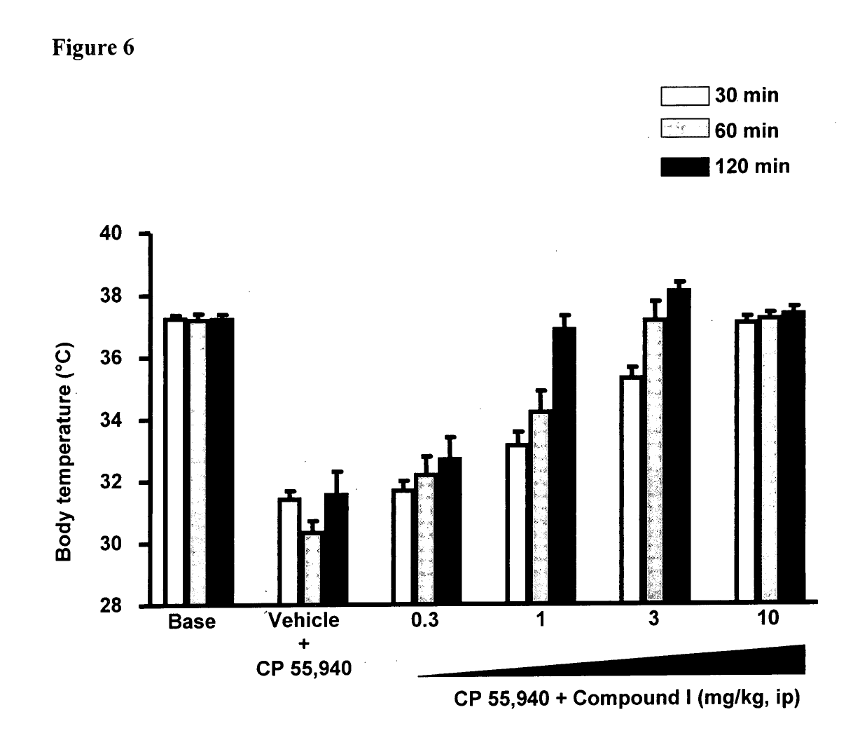

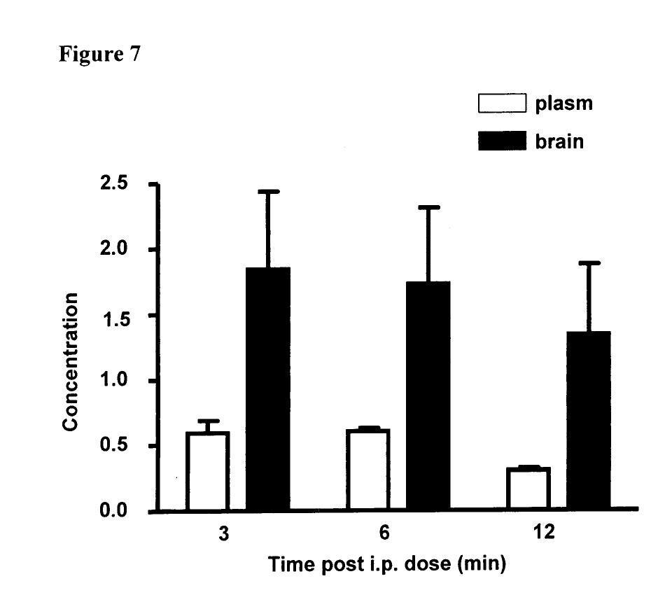

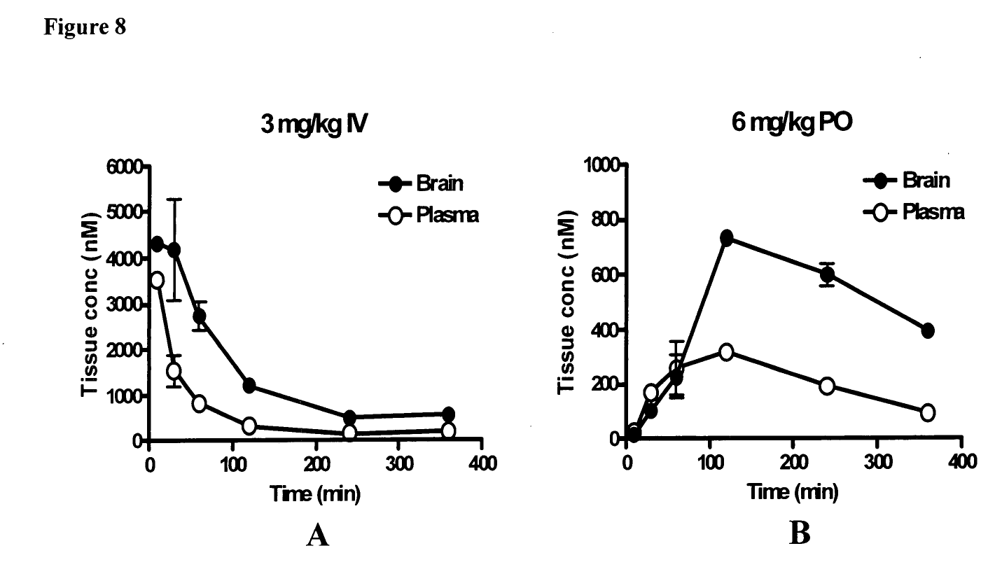

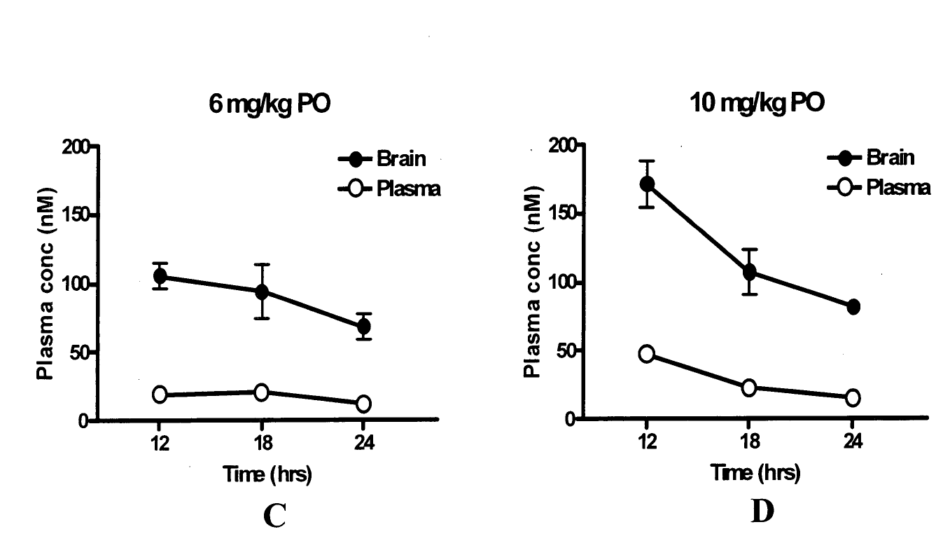

FIG. 6 is a bar graph showing the body temperature of the rats at several points in time after the rats had been dosed with various doses of CP 55,950 or CP55,950 and Compound I. As shown by FIG. 6, Compound I attenuates CP 55,940-induced hypothermia. In addition, the attenuation of the CP55,940-induced hypothermia was dose-dependent.

### Example 618

### Chronic Feeding Study

Male, obese Zucker rats (400-500 g) served as subjects for these studies. Rats were housed individually and had access to food and water ad libitum. Rats were allowed to acclimate to the vivarium for a period of 3 days, during which body weight and consumption of food and water was monitored. Rats were weighed daily at 1500 hr and then injected with either vehicle or with various doses of the test compound. Daily food and water intakes were also monitored. Food and water bottles were weighed at the time body weights were recorded (i.e., 1350 hr). Vehicle or compound was administered daily for a period of up to 15 days.

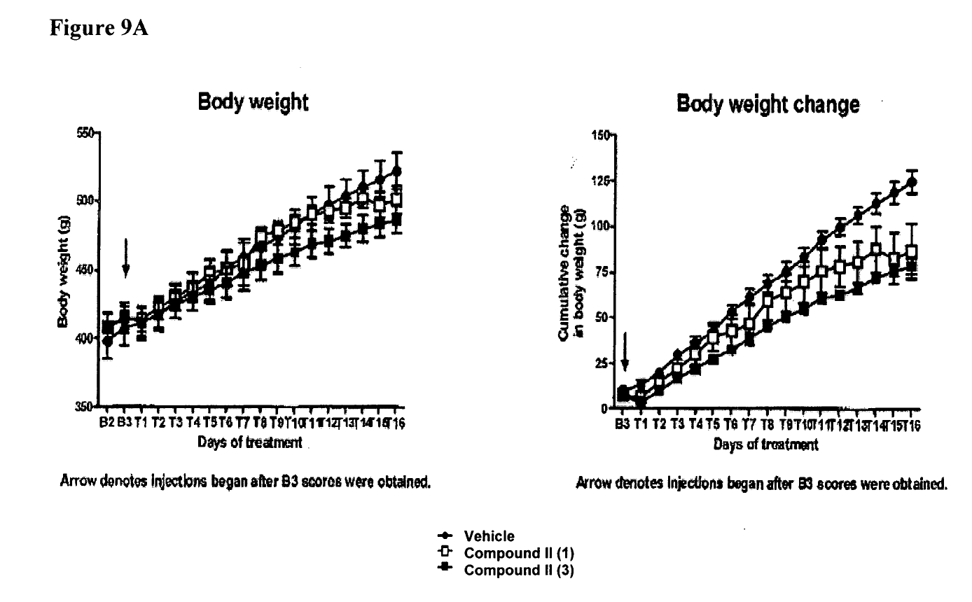

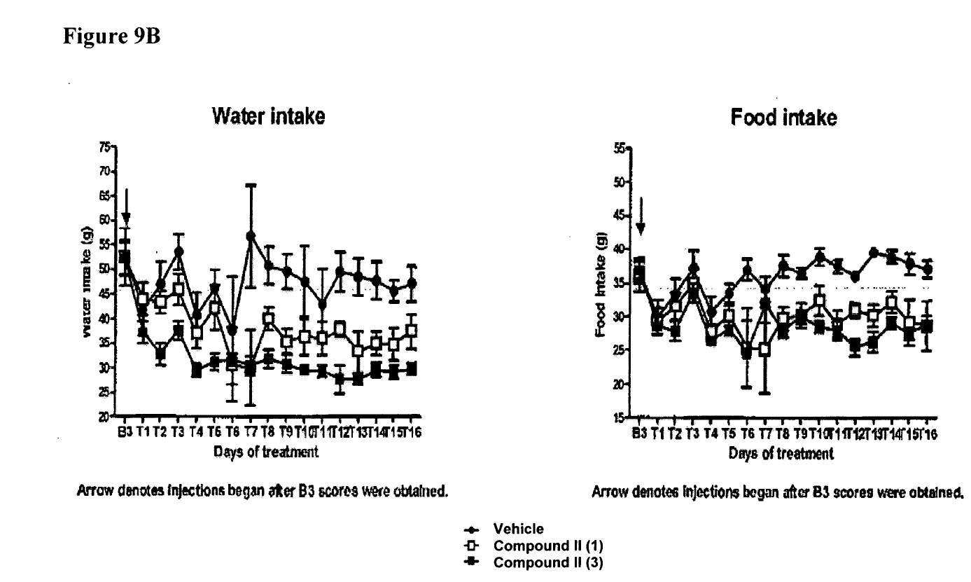

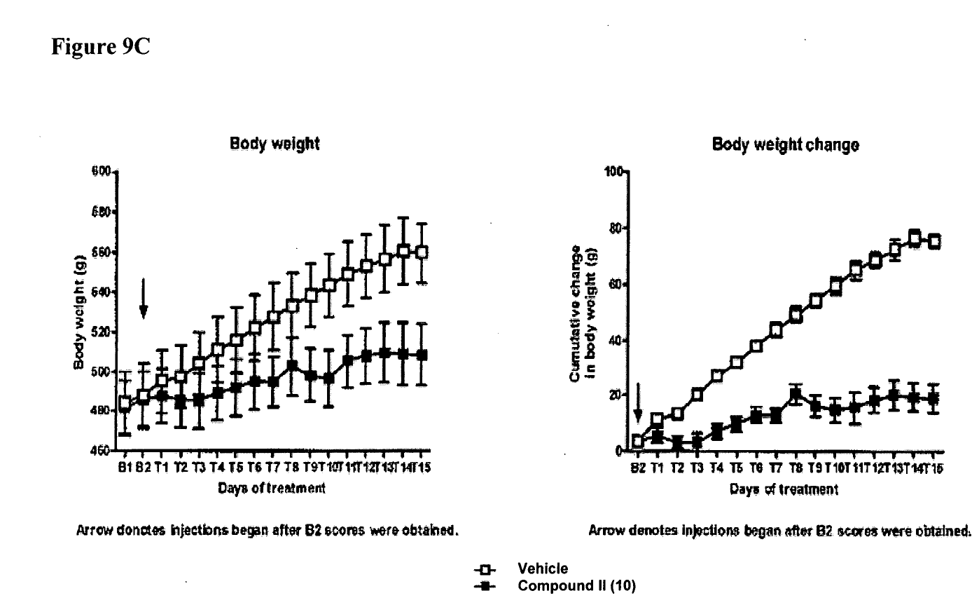

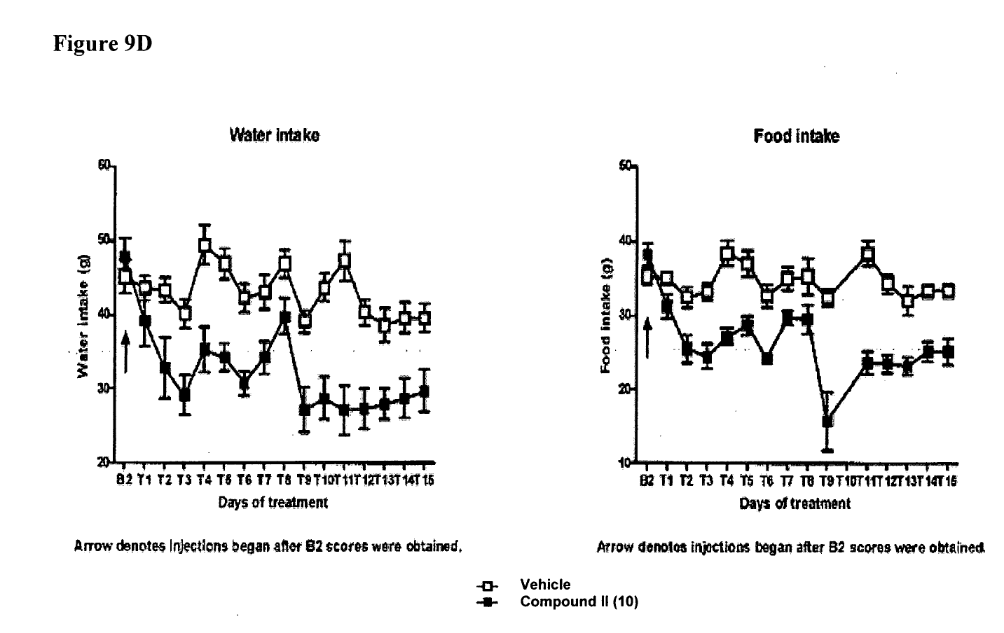

FIG. 9A in a line graph showing the effects of Compound II (1 and 3 mg/kg/day) on body weight FIG. 9B is a line graph showing the effects of Compound II (1 and 3 mg/kg/day) on food intake and water intake. FIG. 9C line graph showing the effects of Compound II (10 mg/kg/day) on body weight. FIG. 9D is a line graph showing the effects of Compound II (10 mg/kg/day) on food intake and water intake. As shown by FIGS. 9A-9D, Compound II attenuated the food and water intake of the rats. Moreover, the attentuation of the food and water intake was dose-dependent.

### Example 619

### Novel Object Recognition Study

Subjects. Subjects were male, C57 BK/6 mice purchased from Harlan Laboratories, weighing 15-20g upon arrival. Animals were housed 8 per cage with food and water available ad libidum. Animals were housed on a 12 hr light cycle (lights on 6 am) for 4-7 days prior to behavioral testing.

Equipment. Novel object recognition (NOR) was conducted in a novel environment consisting of a white plastic tub measuring 45.7×33.7×19 cm. Prior to each trial the bottom of the tub was covered with a piece of plastic lined bench top paper. There were two sets of identical objects chosen so that when given a opportunity to explore, mice would evenly divide exploration time between the objects. “A” objects were yellow, ceramic, 12-sided ramekins measuring 4 cm high×7 cm diameter. “B” objects were 8×8×4 cm stainless steel, 4-sided ramekins.

Procedure. At the beginning of each test day, animals were placed in groups of 6 into clean cages. Testing was conducted in three phases: acclimation, sample and test. For acclimation, each group of six mice was placed collectively into the NOR chamber and allowed to explore freely for 30 min. After acclimation animals were injected (dose and pretreatment time varied by test drug) and placed back into the cages to wait the pre-treatment interval. After the pre-treatment time elapsed, each mouse was placed, one at a time into the NOR chamber, into which two identical objects had been placed (“A” or “B” objects described above). Objects were placed on diagonal corners of the long axis of the arena approximately 5 cm from the walls, while subjects were placed into one of the neutral corners (alternating across subjects). Each mouse was allowed to explore the chamber and the objects for 3 min., and the time spent exploring at each position was recorded. Directly sniffing or touching the object was recorded as exploration. After 3 min., each mouse was removed from the arena and placed back into its cage. The test phase was conducted 1 or 2 hours after the sample phase. During test, one familiar object (seen during sample) and one novel object were placed into the chamber in the same positions used during the sample phase, and each mouse was allowed 3 min to explore. The test sessions were recorded on video and scored by an observer blind to each subject's treatment condition. Any time spent directly sniffing or touching an object was counted as exploration. The object serving as the novel object and the position where the novel object was placed were counterbalanced across subjects. Prior to each trial (acclimation, sample and test), all equipment was wiped with a Clorox wipe and bench paper (cut to fit) was placed in the bottom of the chamber. The procedure is shown below in Scheme 9.

Measures. In addition to time spent exploring each object (T_{N}=time spent exploring novel object, T_{F}=time spent exploring familiar object), two measures were determined for each subject: exploration ratio (% of time spent exploring at novel object) ER=T_{N}*100/(T_{N}+T_{F}) and discrimination index (preference for novel) DI=(T_{N}−T_{F})/(T_{N}+T_{F}).

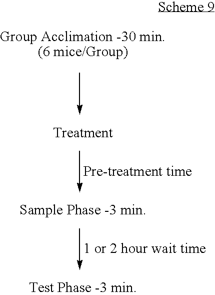

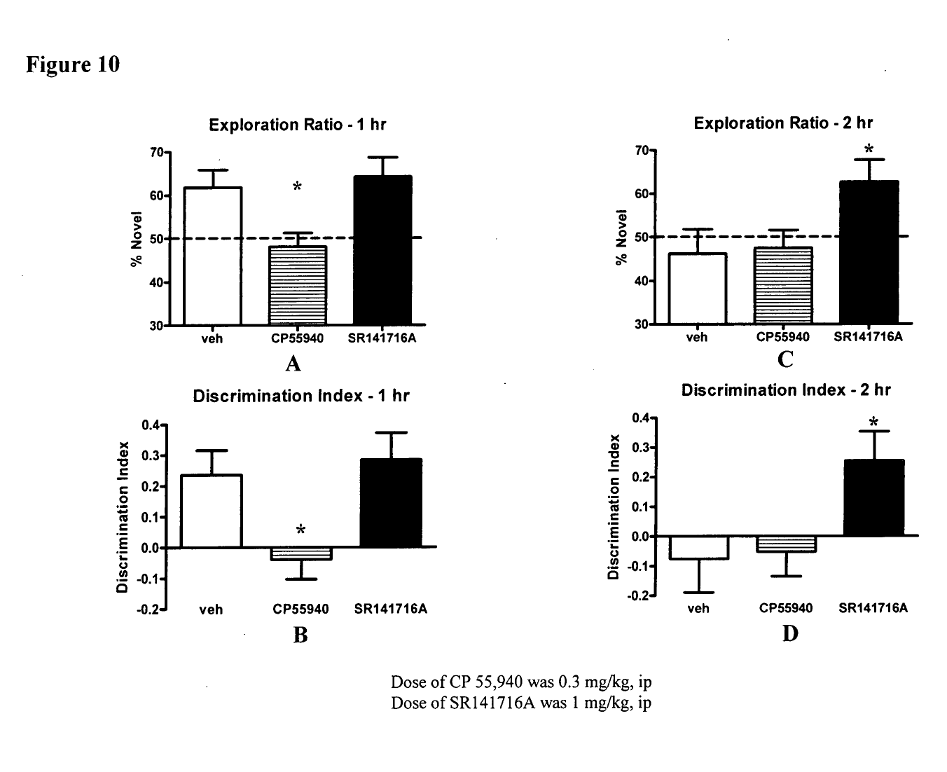

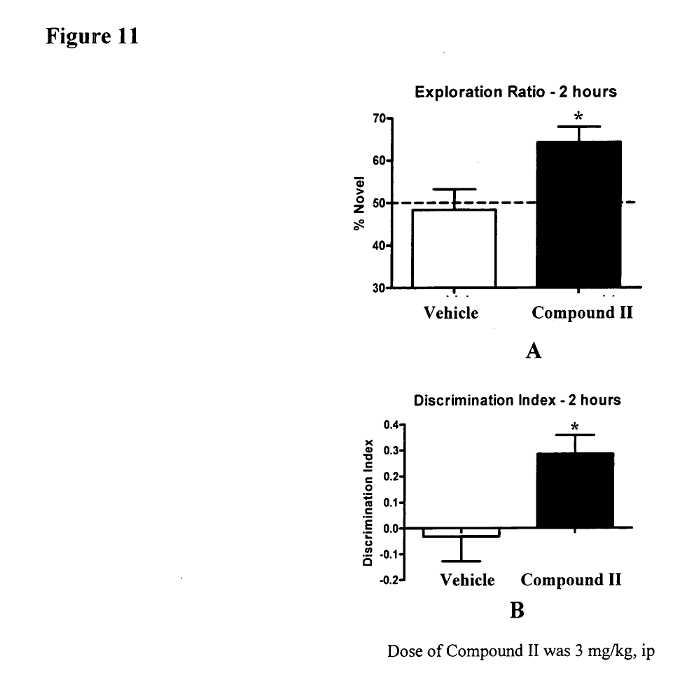

FIGS. 10A and 10C are bar graphs showing the exploration ratio at 1 and 2 hours after the mice had been dosed with the vehicle, CP 55,940 (0.3 mg/kg, ip), or SR141716A (1 mg/kg, ip). FIGS. 10B and 10D are bar graphs showing the discrimination index at 1 and 2 hours after the mice had been dosed with the vehicle, CP 55,940 (0.3 mg/kg, ip), or SR141716A (1 mg/kg, ip). FIG. 11A is a bar graph showing the exploration ratio 2 hours after the mice had been dosed with Compound II (3 mg/kg, ip). FIG. 11B is a bar graph showing the discrimination index 2 hours after the mice had been dosed with Compound II (3 mg/kg, ip).

As shown by FIGS. 10A-D and FIGS. 11A-B, mice treated with SR141716A and Compound II showed a preference for the novel object (indicating the mice recognized the familiar object) up to two hours after being dosed with the test compound. Mice treated with the vehicle or CP 55,940 showed a preference for the novel object after 1 hour of being dosed with the test compound but then returned back to baseline exploration rates after 2 hours.

### Example 620

### Radial Arm Maze Study

Subjects: Subjects for the radial arm maze experiments were male, Sprague-Dawley rats purchased Charles Rivers Laboratories, weighing 225-250 g upon arrival, housed two per cage. All subjects had free access to food and water available for the duration of the study. Animals were housed on a 12 hr light cycle (lights on 7 am), and were acclimated to vivarium conditions for a minimum of two days prior to behavioral training. All experiments were conducted in accordance with NIH Guidelines for the Care and Use of Laboratory Animals and were approved by the Institutional Animal Care and Use Committee at ACADIA Pharmaceuticals, Inc.

Radial Arm Maze Procedure: Radial arm maze (RAM) testing was conducted in a watertight maze (61.0 cm high) made of black ABS plastic, consisting of a central, round chamber (57.1 cm in diameter) with 8 (38.1 cm×16.6 cm) equally spaced arms radiating from the center. The testing room had salient environmental cues that remained constant throughout testing, including a door, a table, a shelving unit, a solid black panel one wall, a black and white striped panel on the opposite wall, and the experimenter seated behind the start arm. Prior to each session, escape platforms were placed in the ends of 6 arms. Escape platforms were made of black ABS plastic (10.1 cm×15.2 cm) covered with Velcro fitted 16 cm from the top of the maze. Each day the maze was filled with water (25° C.) until the platforms were hidden with 1 cm of water covering the platforms. Additionally, non-toxic black paint was dissolved in the water to help visually obscure the platforms and ensure animals could not depend on visual cues to solve the task. For each subject, reference arms (arms without platforms) remained constant across training and testing. During a trial, a subject was released from the start arm, facing the center, and allowed 3 min to locate a platform. If the maximum time elapsed, the animal was guided to the nearest platform. Once a platform was found, animals remained on it for 15 sec before being removed from the maze and placed in a warmed holding tub for 30 sec. During the interval, the chosen platform was removed from the maze. The animal was then returned to the maze for another trial. This continued until all platforms were located. Training was conducted 5 days per week for 10 days. After training, animals began the test phase. During testing, animals received multiple test sessions. In order to ensure adequate time for drug clearance between treatments, subjects received only one test compound and one vehicle treatment per week. In all other respects, test sessions were conducted using the same method described for training.

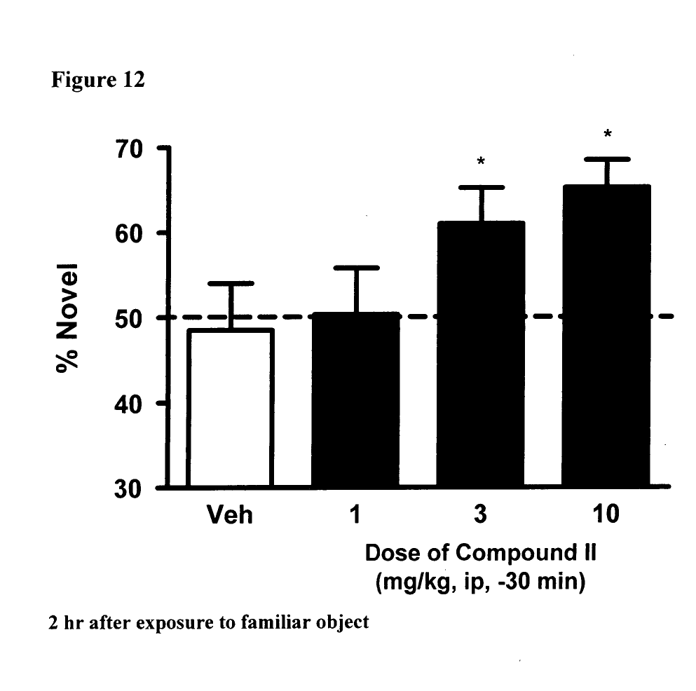

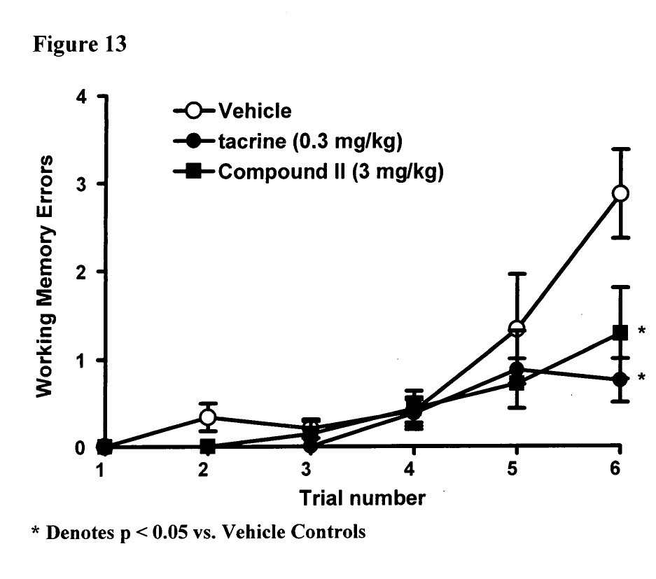

FIG. 12 is a bar graph showing percentage of novel recognition of a familiar object 2 hours after the mice had been dosed with 1, 3, or 10 mg/kg of Compound II. FIG. 13 is a line graph showing the working memory errors of the mice after being dosed with the vehicle, tacrine (0.3 mg/kg), or Compound II (3 mg/kg).

As shown by FIGS. 12 and 13, mice treated with Compound II showed a preference for the novel object (indicating the mice recognized the familiar object) up to two hours after being dosed with the test compound.

### Example 621

### Rotation Study

Subjects: Subjects were male, Sprague-Dawley rats purchased from Harlan Laboratories, weighing 250-275 g upon arrival. Prior to surgery animals were housed two per cage. All subjects had free access to food and water available for the duration of the study. Animals were housed on a 12 hr light cycle (lights on 6 am), and were acclimated to vivarium conditions for a minimum of one week prior to surgery. All experiments were conducted in accordance with NIH Guidelines for the Care and Use of Laboratory Animals and were approved by the Institutional Animal Care and Use Committee at ACADIA Pharmaceuticals, Inc.

Surgery. One week after arrival, subjects underwent stereotaxic surgery to unilaterally lesion dopamine terminals within the substantia nigra, a common model of Parkinson's disease. In order to protect noradrenergixc terminals, subjects were administered desipramine (20 mg/kg ip) approximately 20 min prior to surgery. Surgery was conducted under ketamine (80 mg/kg ip) and xylazine (12 mg/kg ip) anesthesia. Animals were placed in the stereotaxic instrument with the incisor bar at −3.2 mm and a hole was drilled in the skull over the substantia nigra according to the atlas of Paxinos and Watson (1997): A/P −5.2 mm, MIL −2.1 mm. A computer-controlled microsyringe was lowered to -8.2 mm from bregma. 8 μg of 6-hydroxy-dopamine in 4 μl of saline with 0.2% ascorbic acid was infused over 5 min, and 1 min was allowed for diffusion before the syringe was removed and the incision closed. Animals were given a minimum of 15 days after surgery before any behavioral assessment.

Rotational Behavior. All animals were assessed for rotational behavior in rotometers purchased from San Diego Instruments, Inc. For each behavioral session, subjects were placed in the rotometers and allowed thirty minutes for acclimation. After 30 min., subjects were injected with either the dopamine agonist apomorphine (0.05, 0.16 or 0.5 mg/kg ip in saline with 0.2% ascorbic acid) or the cannabinoid 1 receptor inverse agonist Compound II, N-(butyl)-11-(4-chlorophenyl)-dibenzo[b,f,][1,4]thiazepine-8-carboxamide, (3 mg/kg in sesame oil). When subjects received combinations of the two treatments, Compound II was injected 30 minutes prior to apomorphine. After treatment, rotations were measured for 60 min. Subjects were then removed from the rotometers and returned to their home cages. All animals received all three doses of apomorphine, and the combination of Compound II with both 0.05 mg/kg and 0.16 mg/kg apomorphine. A minimum of 2 days separated test days.

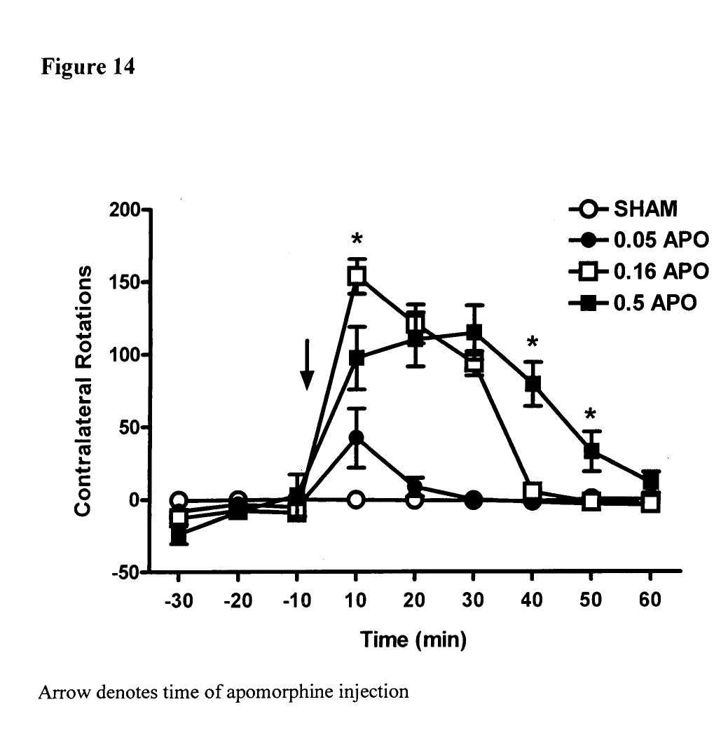

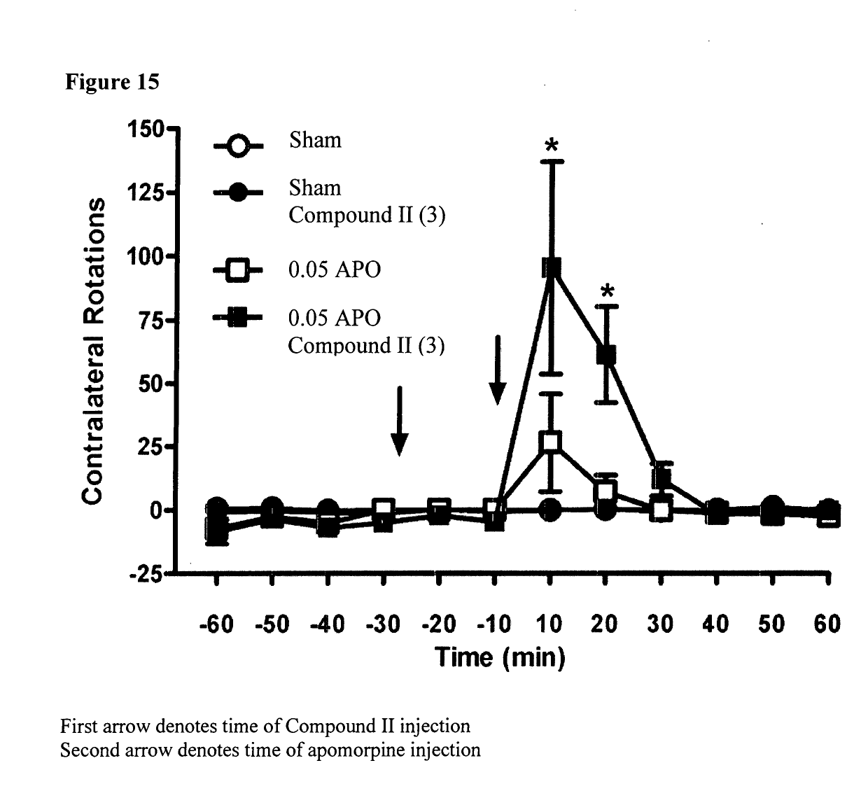

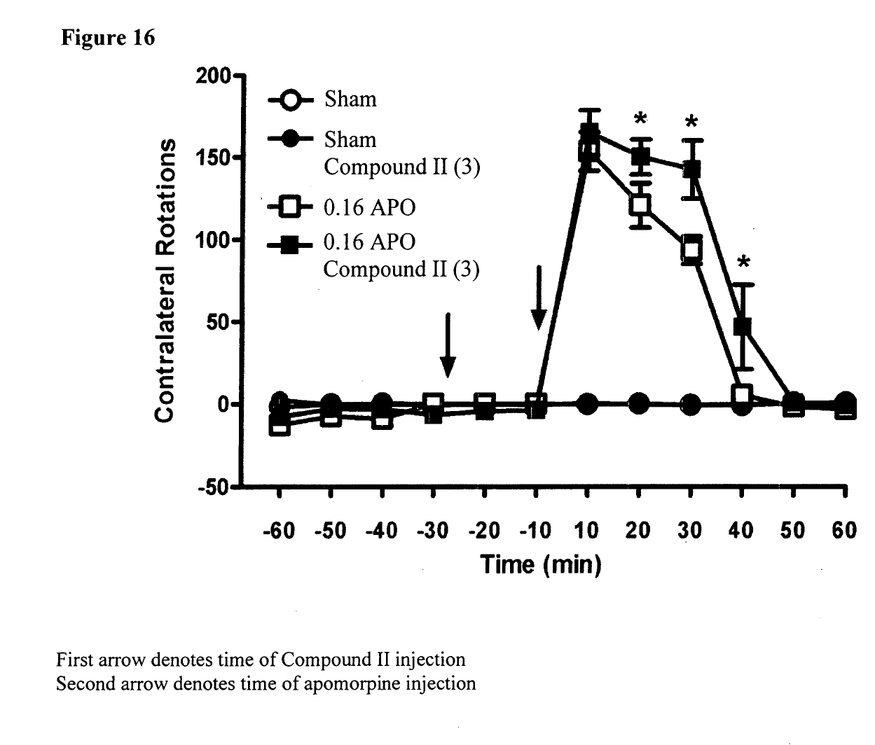

FIG. 14 is a line showing the contralateral rotations over time of the mice after being dosed with apomorphine (0.05, 0.16, and 0.5 mg/kg). FIG. 15 is a line showing the contralateral rotations over time of the mice after being dosed with apomorphine (0.05 mg/kg), Compound II (3.0 mg/kg), or apomorphine (0.05 mg/kg) and Compound II (3.0 mg/kg). FIG. 16 is a line showing the contralateral rotations over time of the mice after being dosed with apomorphine (0.16 mg/kg), Compound II (3.0 mg/kg), or apomorphine (0.16 mg/kg) and Compound II (3.0 mg/kg).

As shown by FIG. 14, apomorphine dose-dependently elicits contralateral rotations in rats with unilateral 6-OH dopamine lesions. FIGS. 15 and 16 show that Compound II augments dopaminergic functions.

Although the invention has been described with reference to the above examples, it will be understood that modifications and variations are encompassed within the spirit and scope of the invention. Accordingly, the invention is limited only by the following claims.

### REFERENCES

The following references are incorporated by reference herein in their entirety:

1. Le Foll B, Goldberg S R. Cannabinoid CBI receptor antagonists as promising new medications for drug dependence. J Pharmacol Exp Ther. March 2005; 312(3):875-83.

2. Boyd S T, Fremming B A. Rimonabant—a selective CB1 antagonist. Ann Pharmacother. April 2005; 39(4):684-90.

3. Howlett A C, Breivogel C S, Childers S R, Deadwyler S A, Hampson R E, Porrino L. J. Cannabinoid physiology and pharmacology: 30 years of progress. Neuropharmacology. 2004; 47 Suppl 1:345-58.

APPENDIX A

| Compound | pIC₅₀ | % Inhibition |
|---|---|---|
| 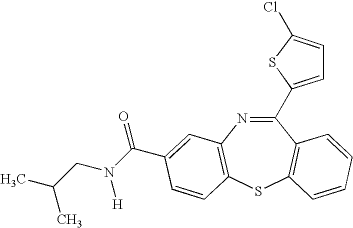 | 8.7 | 7.4 |
| 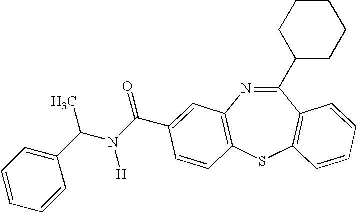 | 7.2 | 87 |
| 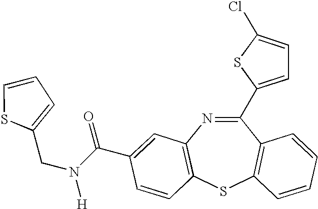 | 8.7 | 114 |
| 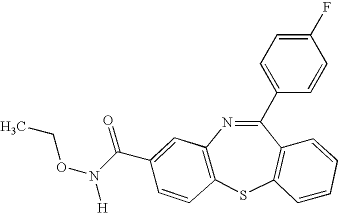 | 7.2 | 98 |
| 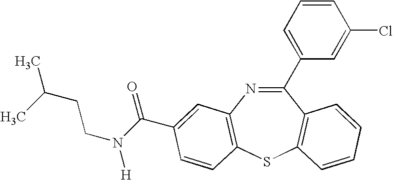 | 8.6 | 115 |
| 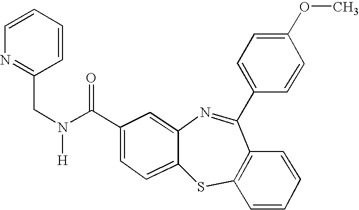 | 7.2 | 93 |
| 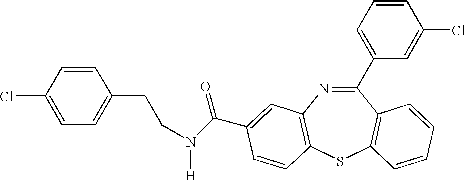 | 8.6 | 106 |
| 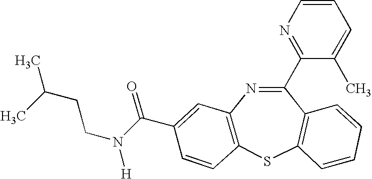 | 7.2 | 94 |
| 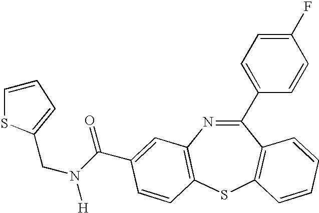 | 8.5 | 97 |
| 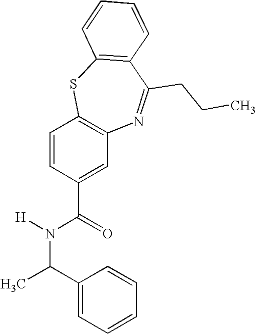 | 7.2 | 97 |
| 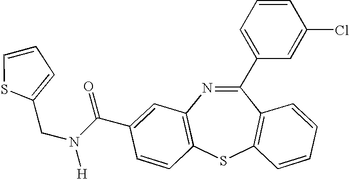 | 8.6 | 108 |
| 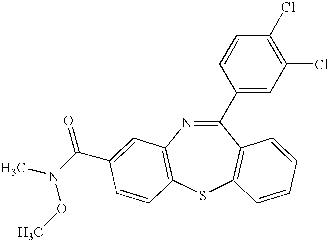 | 7.2 | 0 |
| 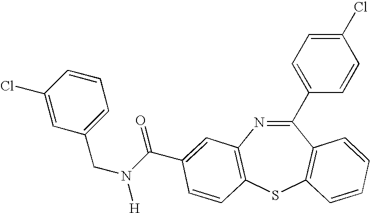 | 8.5 | 112 |
| 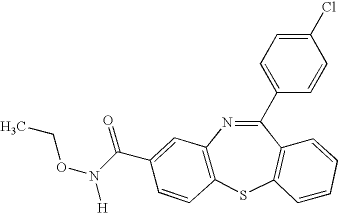 | 7.2 | 91 |
| 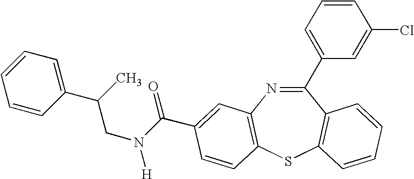 | 8.5 | 112 |
| 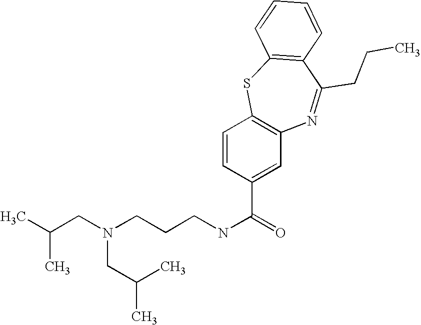 | 7.2 | 103 |
| 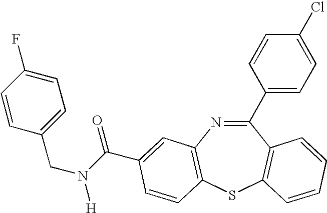 | 8.5 | 106 |
| 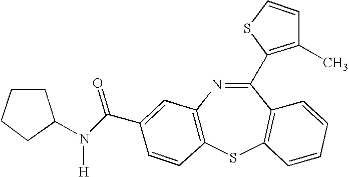 | 7.2 | 81 |
| 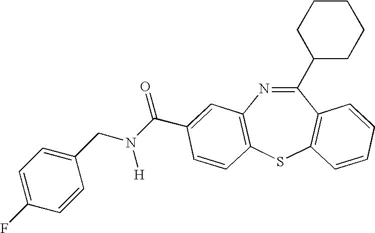 | 8.5 | 102 |
| 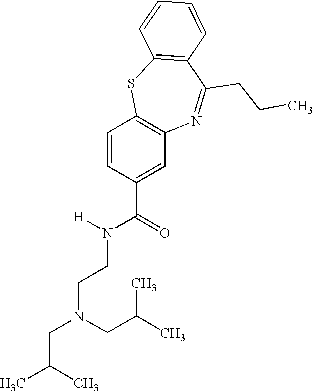 | 7.2 | 105 |
| 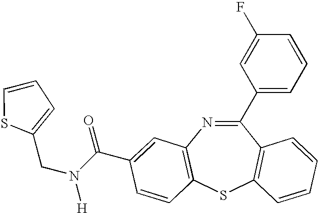 | 8.5 | 93 |
| 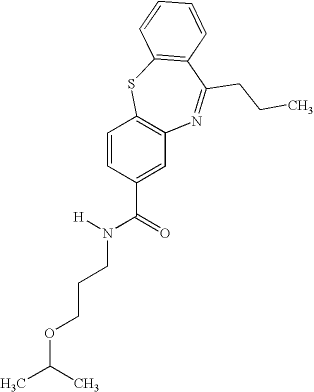 | 7.2 | 117 |
| 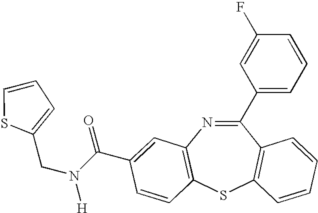 | 8.5 | 100 |
| 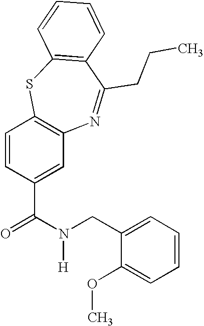 | 7.2 | 91 |
| 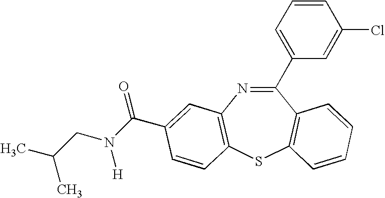 | 8.4 | 116 |
| 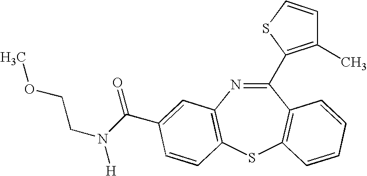 | 7.2 | 88 |
| 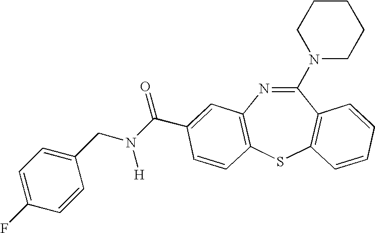 | 8.4 | 80 |
| 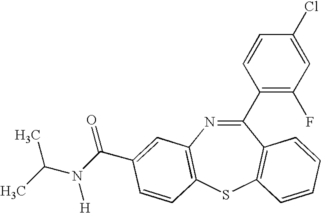 | 7.2 | 101 |
| 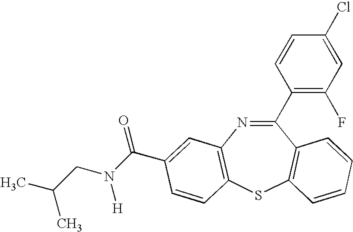 | 8.4 | 110 |
| 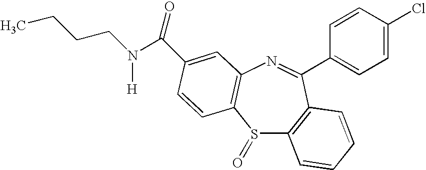 | 7.2 | 97 |
| 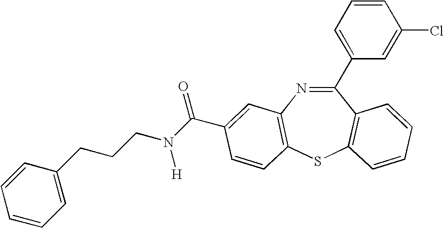 | 8.4 | 104 |
| 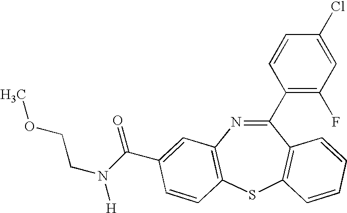 | 7.2 | 104 |
| 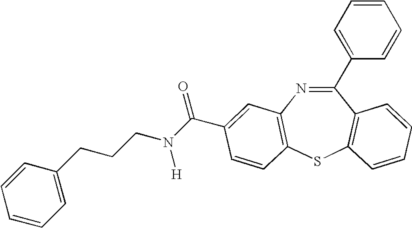 | 8.4 | 111 |
| 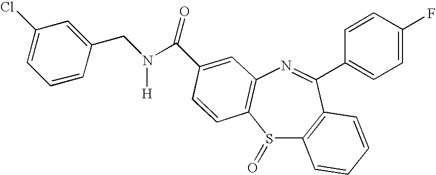 | 7.2 | 89 |
| 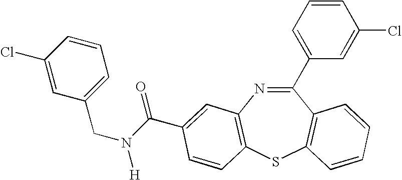 | 8.4 | 112 |
| 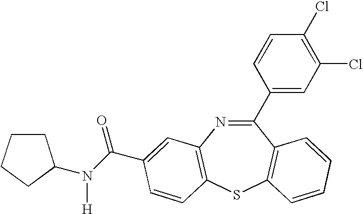 | 7.2 | 95 |
| 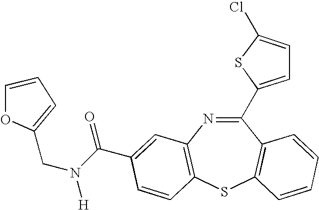 | 8.4 | 118 |
| 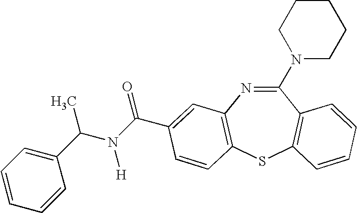 | 7.2 | 113 |
| 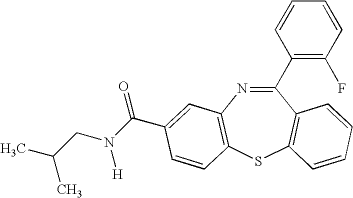 | 8.3 | 118 |
| 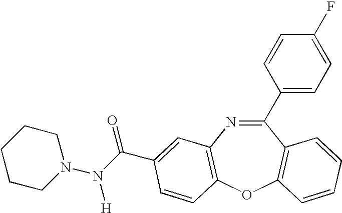 | 7.2 | 84 |
| 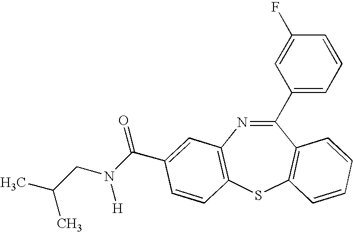 | 8.3 | 112 |
| 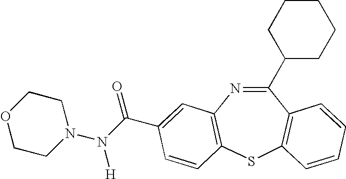 | 7.2 | 56 |
| 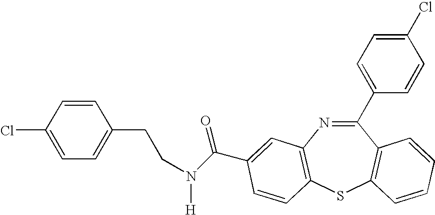 | 8.3 | 104 |
| 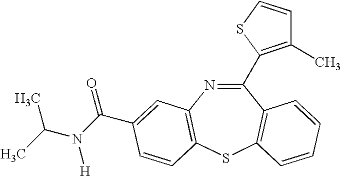 | 7.2 | 95 |
| 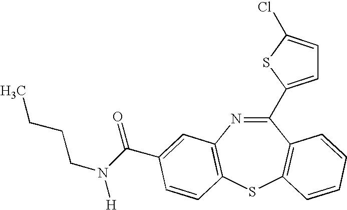 | 8.3 | 92 |
| 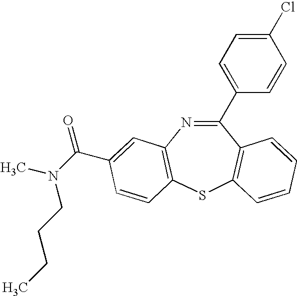 | 7.2 | 92 |
| 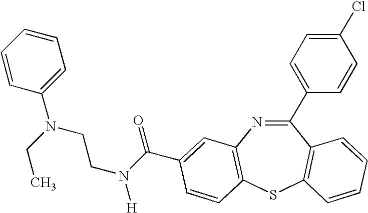 | 8.3 | 102 |
| 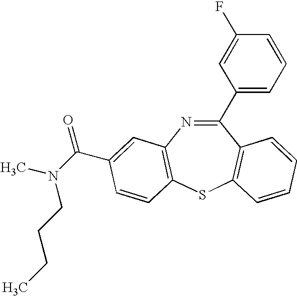 | 7.2 | 90 |
| 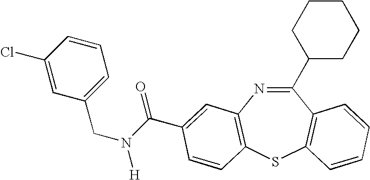 | 8.3 | 112 |
| 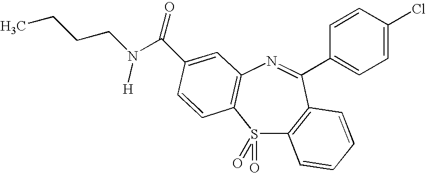 | 7.2 | 106 |
| 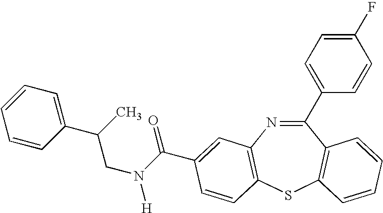 | 8.3 | 98 |
| 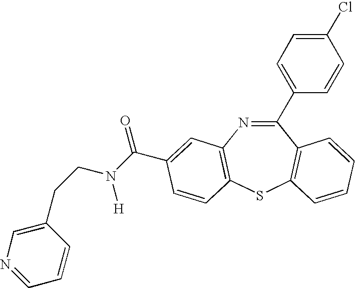 | 7.2 | 116 |
| 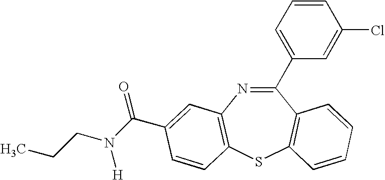 | 8.3 | 107 |
| 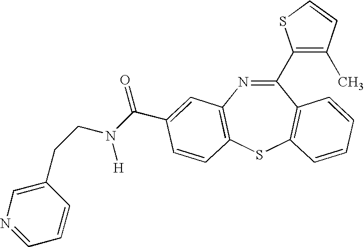 | 7.2 | 121 |
| 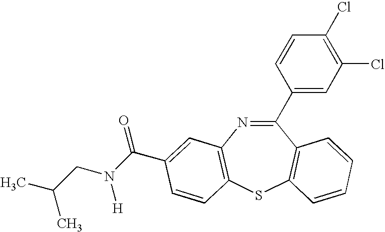 | 8.3 | 108 |
| 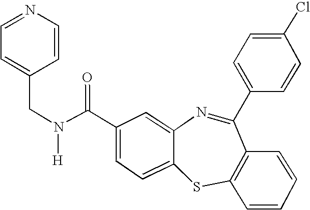 | 7.1 | 105 |
| 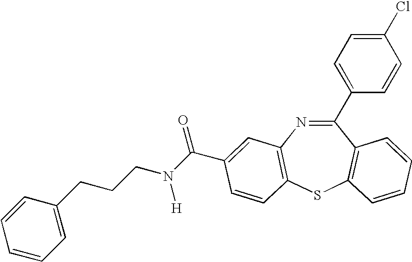 | 8.3 | 106 |
| 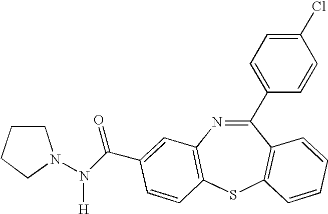 | 7.1 | 100 |
| 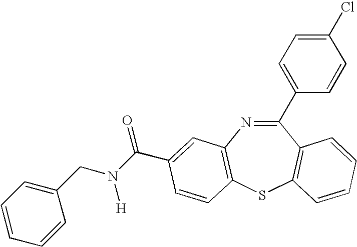 | 8.3 | 105 |
| 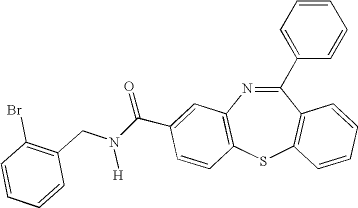 | 7.1 | 83 |
| 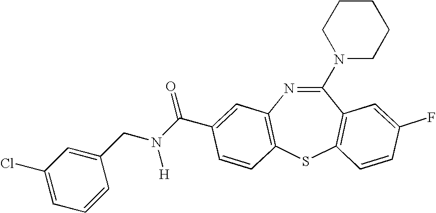 | 8.3 | 107 |
| 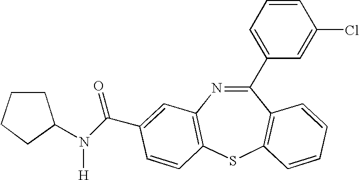 | 7.1 | 96 |
| 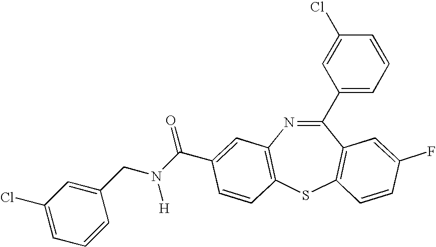 | 8.3 | 107 |
| 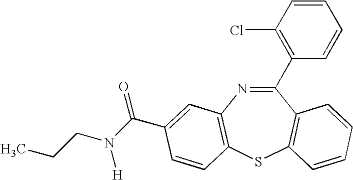 | 7.1 | 78 |
| 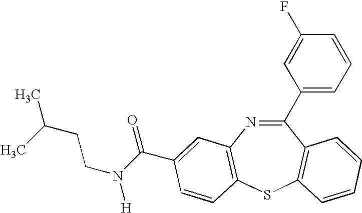 | 8.2 | 100 |
| 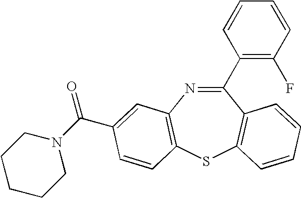 | 7.1 | 73 |
| 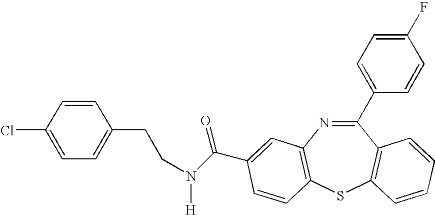 | 8.2 | 107 |
| 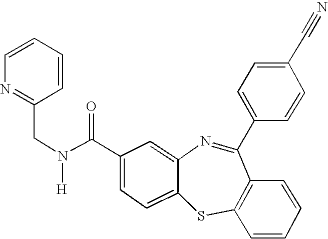 | 7.1 | 99 |
| 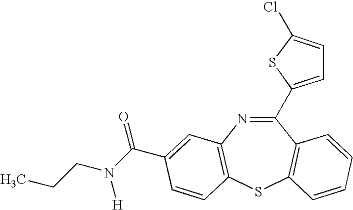 | 8.2 | 88 |
| 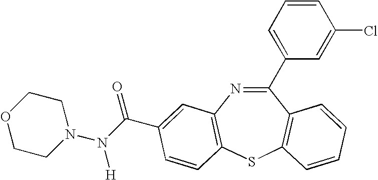 | 7.1 | 80 |
| 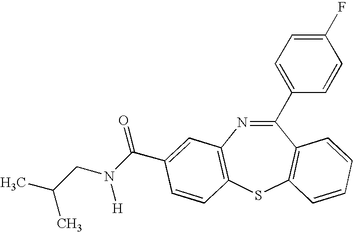 | 8.2 | 107 |
| 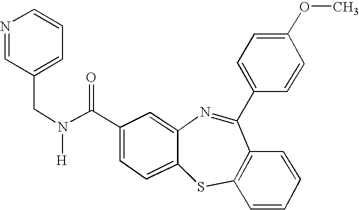 | 7.1 | 106 |
| 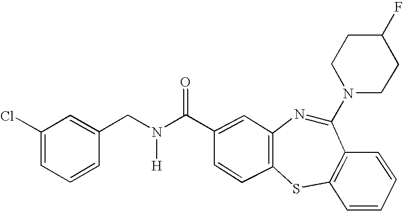 | 8.2 | 108 |
| 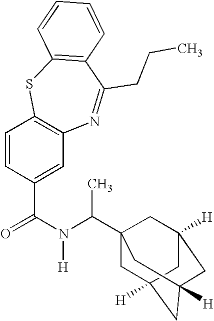 | 7.1 | 112 |
| 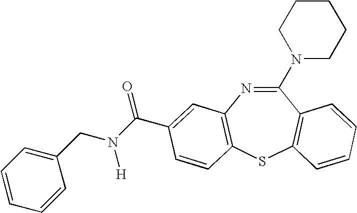 | 8.2 | 110 |
| 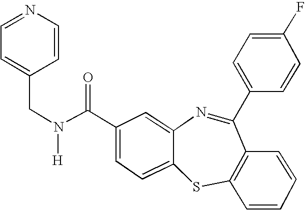 | 7.1 | 101 |
| 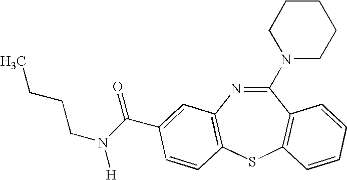 | 8.2 | 99 |
| 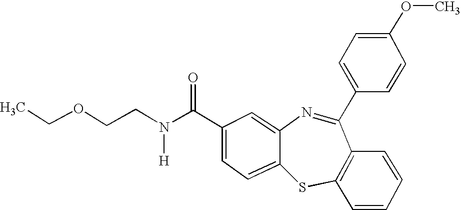 | 7.1 | 90 |
| 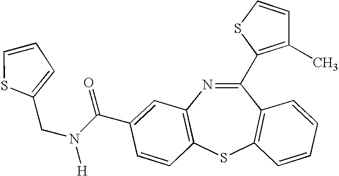 | 8.2 | 114 |
| 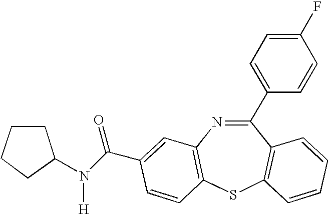 | 7.1 | 117 |
| 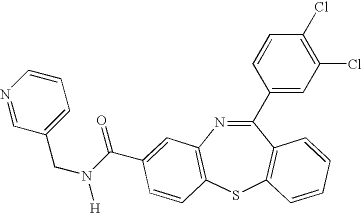 | 8.2 | 114 |
| 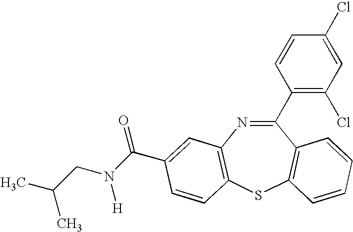 | 7.1 | 87 |
| 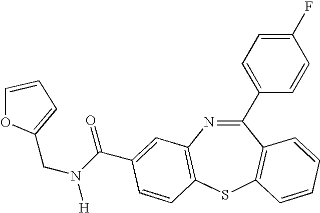 | 8.2 | 104 |
| 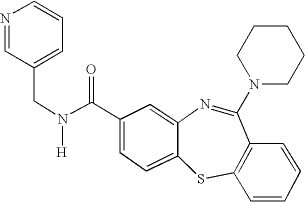 | 7.1 | 82 |
| 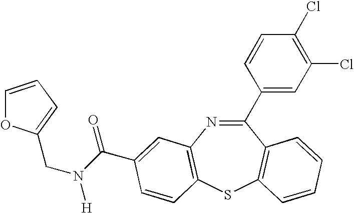 | 8.2 | 97 |
| 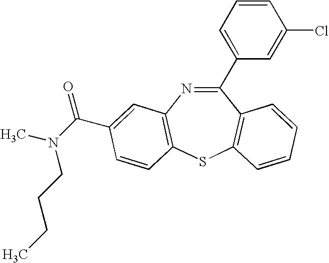 | 7.1 | 82 |
| 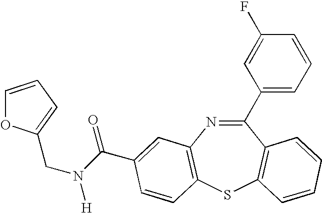 | 8.2 | 104 |
| 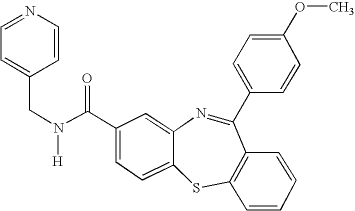 | 7.1 | 105 |
| 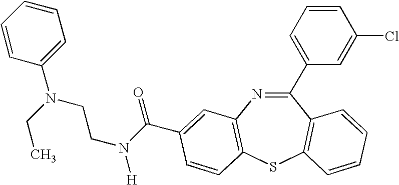 | 8.1 | 97 |
| 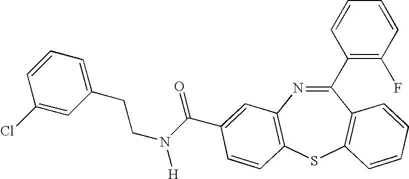 | 7.1 | 91 |
| 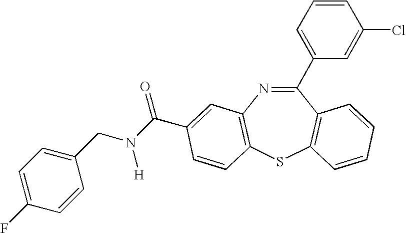 | 8.1 | 97 |
| 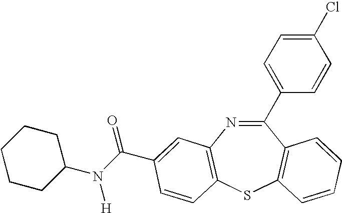 | 7.1 | 96 |
| 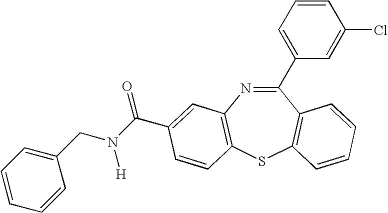 | 8.1 | 106 |
| 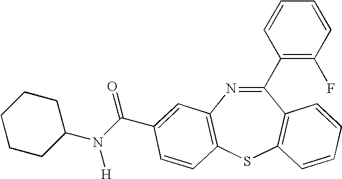 | 7.1 | 109 |
| 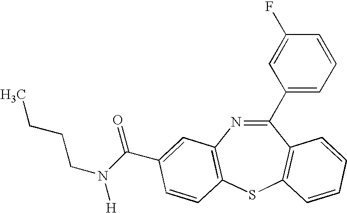 | 8.1 | 113 |
| 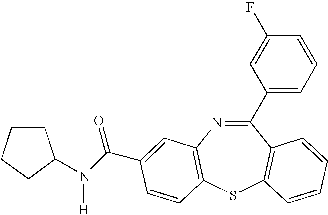 | 7.1 | 100 |
| 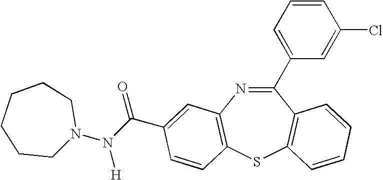 | 8.1 | 105 |
| 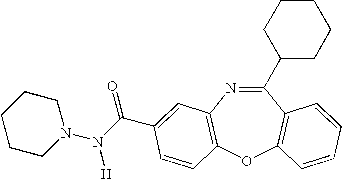 | 7.1 | 86 |
| 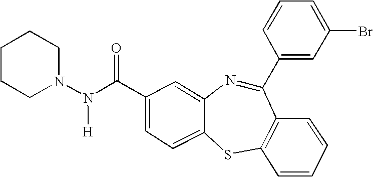 | 8.1 | 92 |
| 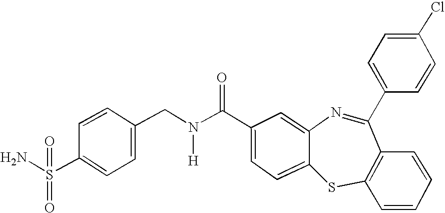 | 7.1 | 76 |
| 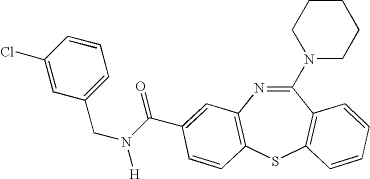 | 8.1 | 85 |
| 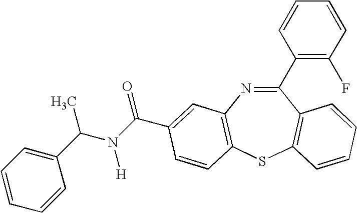 | 7.1 | 69 |
| 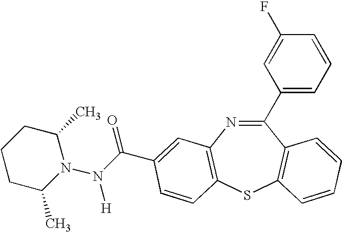 | 8.1 | 120 |
| 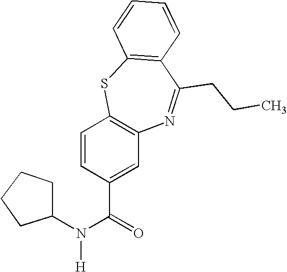 | 7.1 | 109 |
| 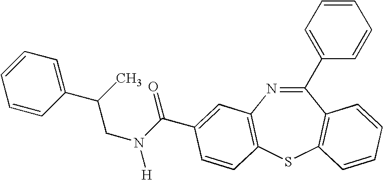 | 8.1 | 101 |
| 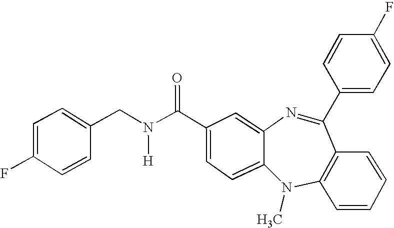 | 7.1 | 91 |
| 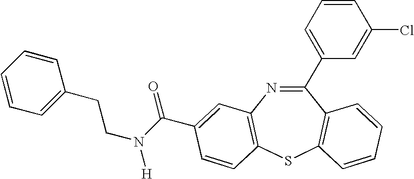 | 8.1 | 104 |
| 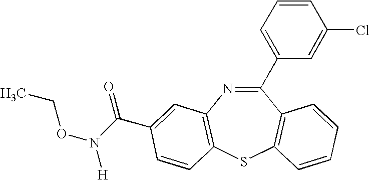 | 7.1 | 90 |
| 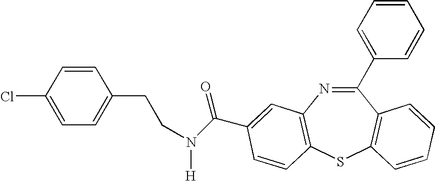 | 8.1 | 108 |
| 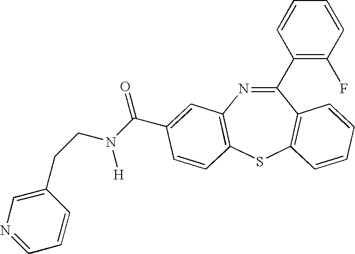 | 7.1 | 110 |
| 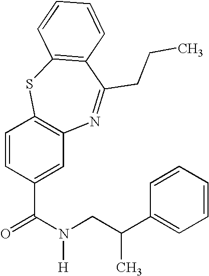 | 8.1 | 104 |
| 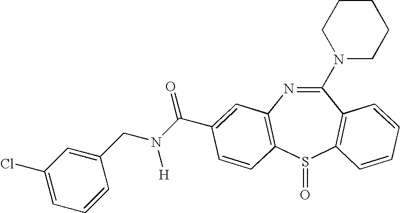 | 7.0 | 71 |
| 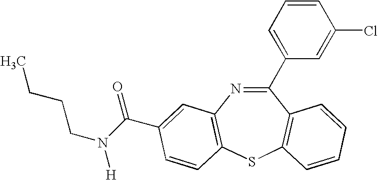 | 8.1 | 98 |
| 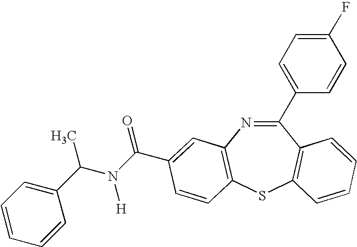 | 7.0 | 81 |
| 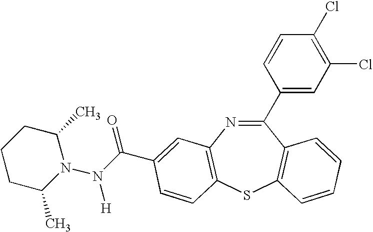 | 8.1 | 114 |
| 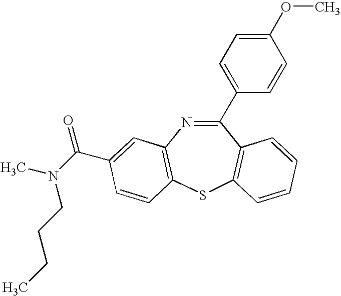 | 7.0 | 97 |
| 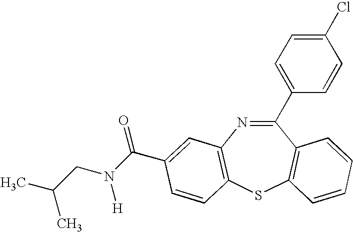 | 8.1 | 123 |
| 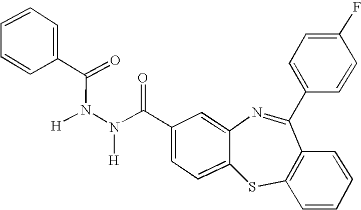 | 7.0 | 83 |
| 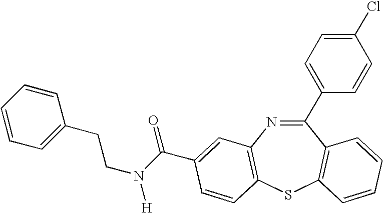 | 8.1 | 110 |
| 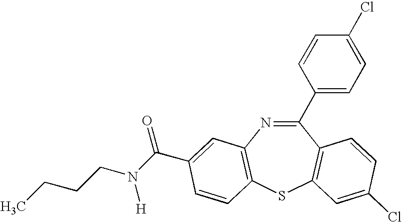 | 7.0 | 90 |
| 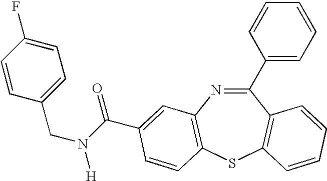 | 8.1 | 108 |
| 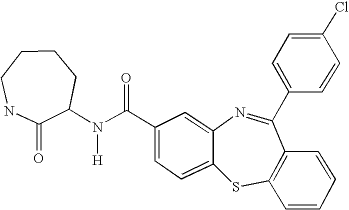 | 7.0 | 81 |
| 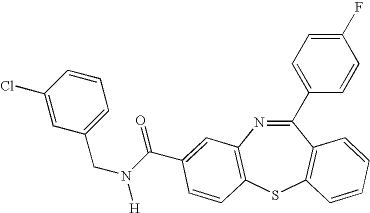 | 8.1 | 106 |
| 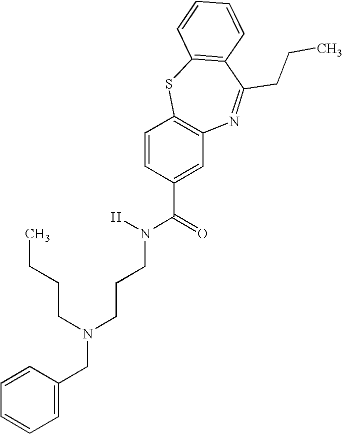 | 7.0 | 99 |
| 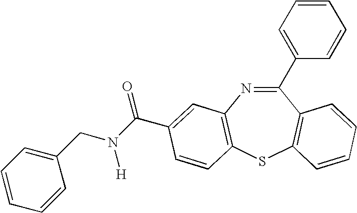 | 8.0 | 111 |
| 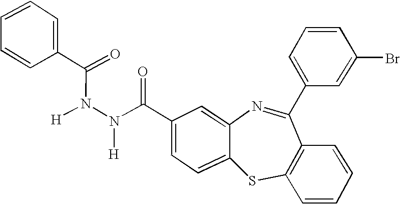 | 7.0 | 76 |
| 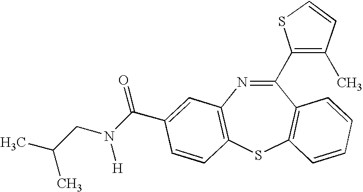 | 8.0 | 88 |
| 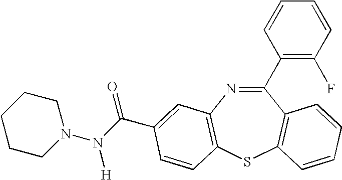 | 7.0 | 104 |
| 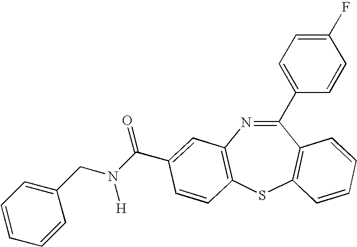 | 8.0 | 96 |
| 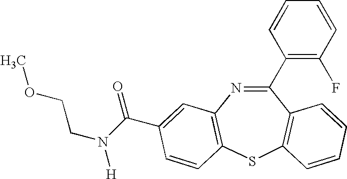 | 7.0 | 99 |
| 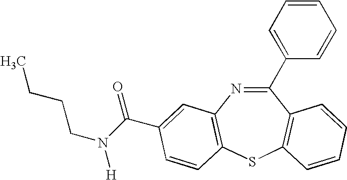 | 8.0 | 104 |
| 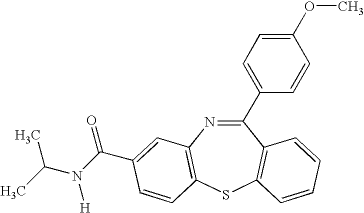 | 7.0 | 84 |
| 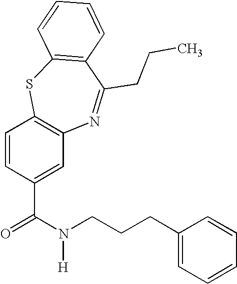 | 8.0 | 104 |
| 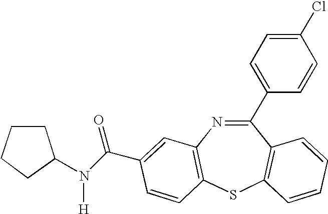 | 7.0 | 105 |
| 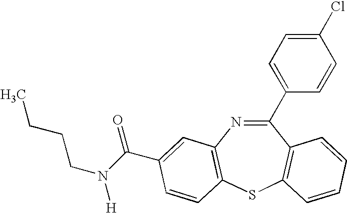 | 8.1 | 110 |
| 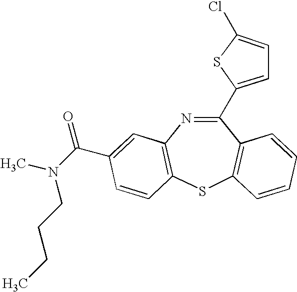 | 7.0 | 87 |
| 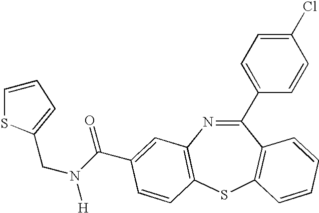 | 8.1 | 107 |
| 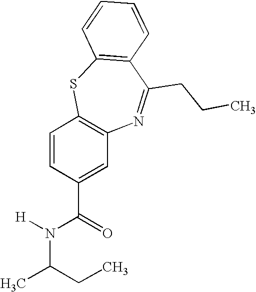 | 7.0 | 105 |
| 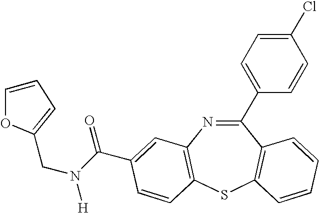 | 8.1 | 94 |
| 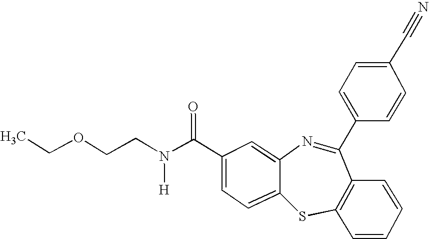 | 7.0 | 74 |
| 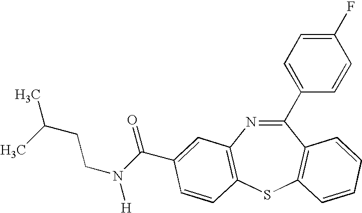 | 8.0 | 107 |
| 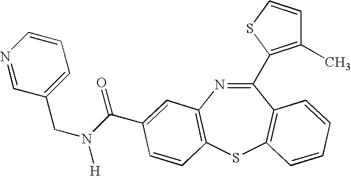 | 7.0 | 102 |
| 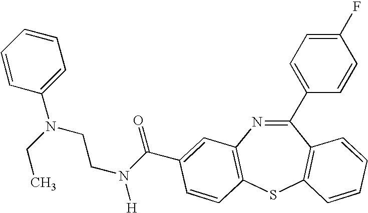 | 8.0 | 99 |
| 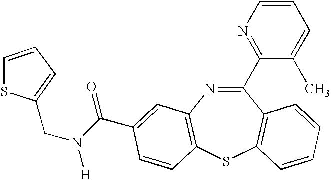 | 7.0 | 97 |
| 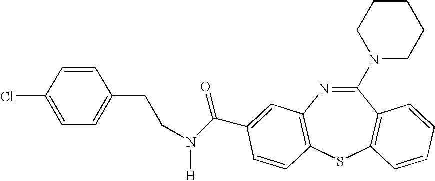 | 8.0 | 113 |
| 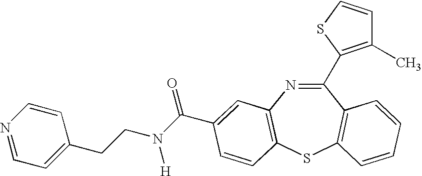 | 7.0 | 101 |
| 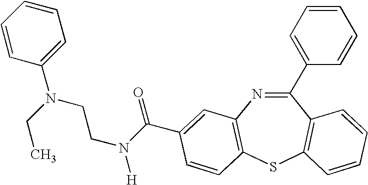 | 8.0 | 106 |
| 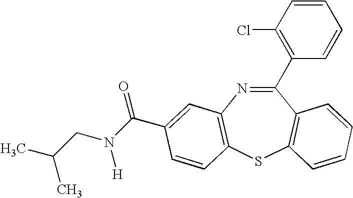 | 6.9 | 86 |
| 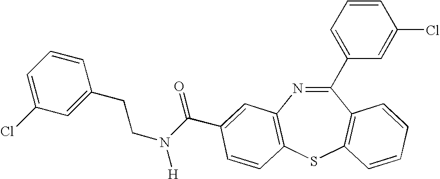 | 8.0 | 107 |
| 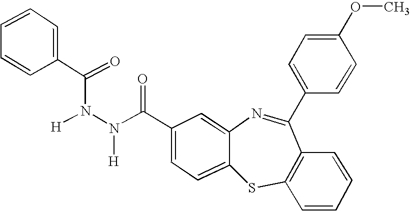 | 6.9 | 83 |
| 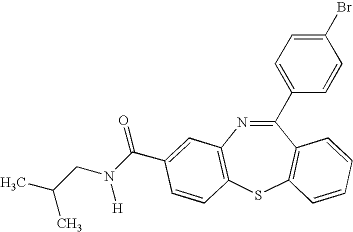 | 8.0 | 80 |
| 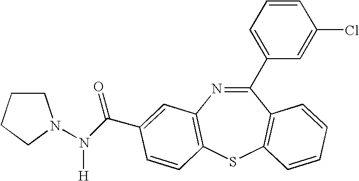 | 6.9 | 85 |
| 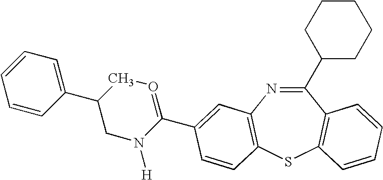 | 8.0 | 100 |
| 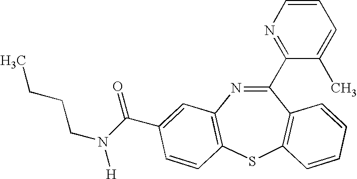 | 6.9 | 85 |
| 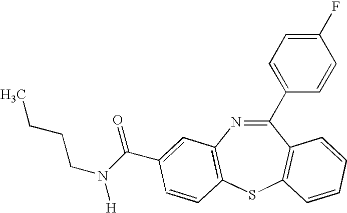 | 8.0 | 96 |
| 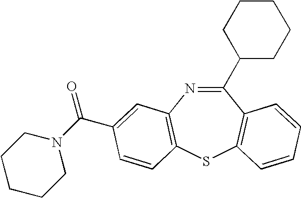 | 6.9 | 26 |
| 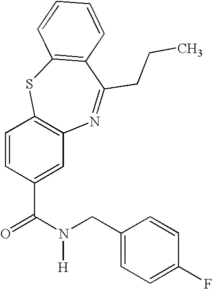 | 8.0 | 107 |
| 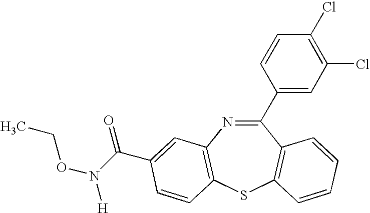 | 6.9 | 87 |
| 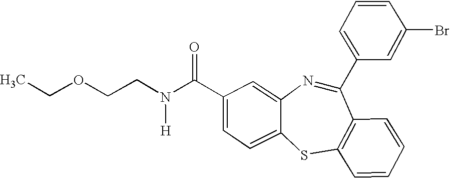 | 8.0 | 119 |
| 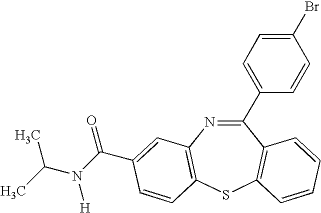 | 6.9 | 83 |
| 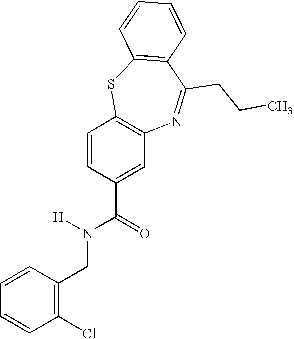 | 8.0 | 105 |
| 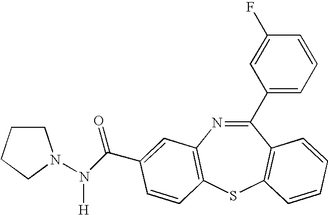 | 6.9 | 67 |
| 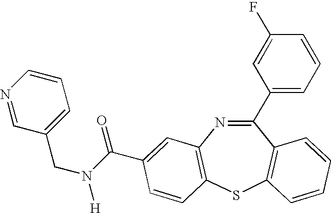 | 8.0 | 130 |
| 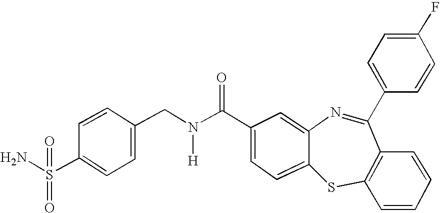 | 6.9 | 83 |
| 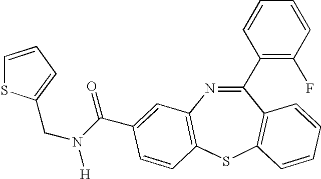 | 8.0 | 114 |
| 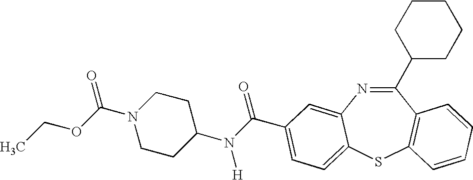 | 6.9 | 95 |
| 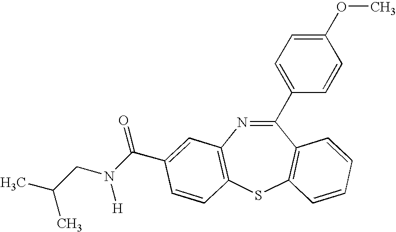 | 7.9 | 118 |
| 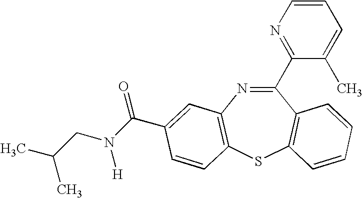 | 6.9 | 87 |
| 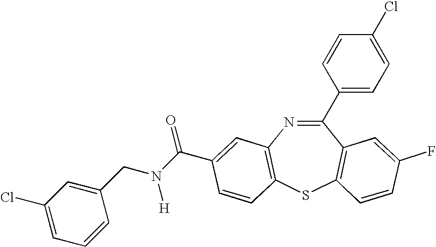 | 7.9 | 110 |
| 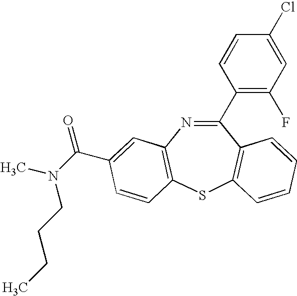 | 6.9 | 84 |
| 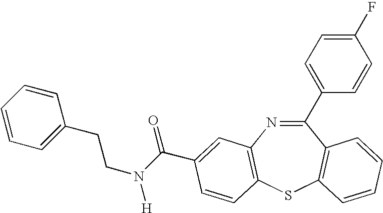 | 7.9 | 103 |
| 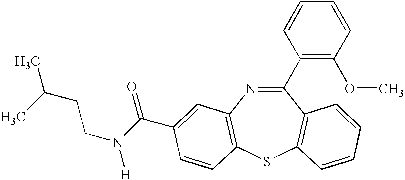 | 6.9 | 91 |
| 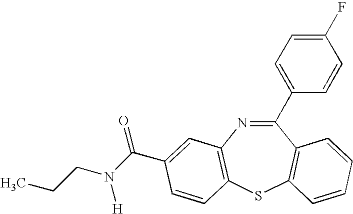 | 7.9 | 95 |
| 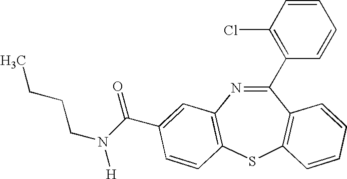 | 6.9 | 89 |
| 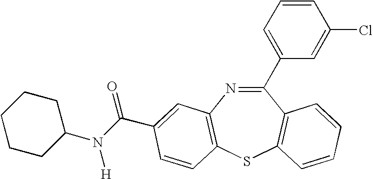 | 7.9 | 99 |
| 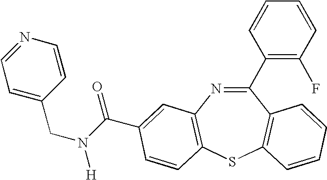 | 6.9 | 94 |
| 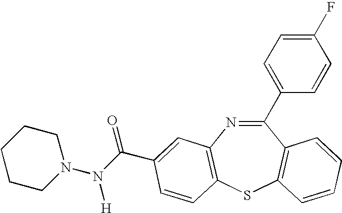 | 7.9 | 97 |
| 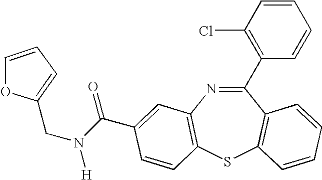 | 6.9 | 95 |
| 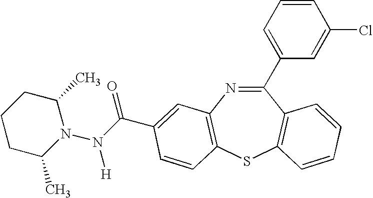 | 7.9 | 100 |
| 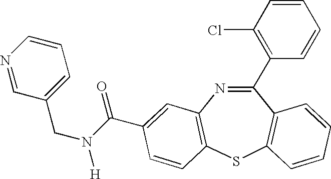 | 6.9 | 72 |
| 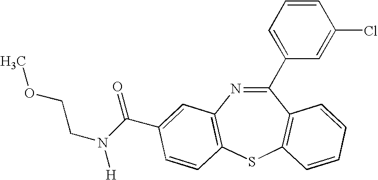 | 7.9 | 110 |
| 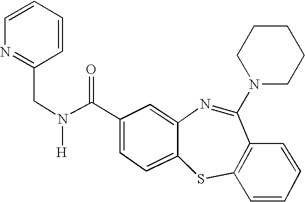 | 6.8 | 78 |
| 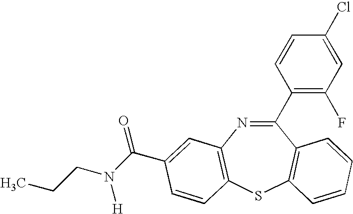 | 7.9 | 101 |
| 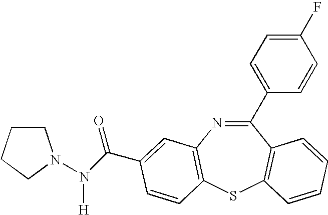 | 6.8 | 92 |
| 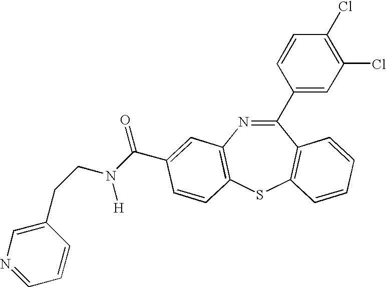 | 7.9 | 104 |
| 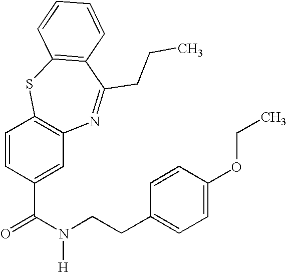 | 6.8 | 102 |
| 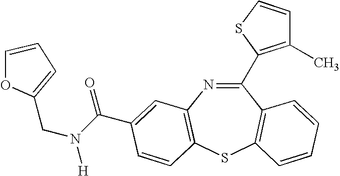 | 7.9 | 100 |
| 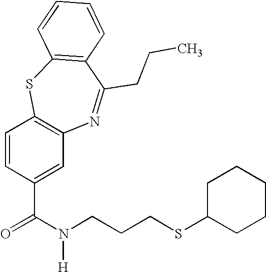 | 6.8 | 95 |
| 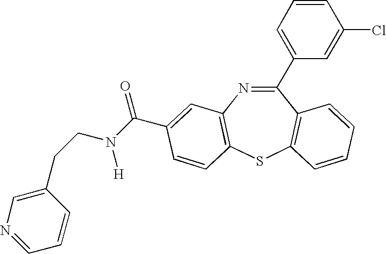 | 7.9 | 100 |
| 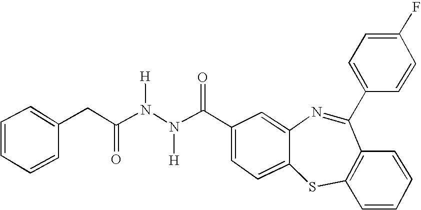 | 6.8 | 98 |
| 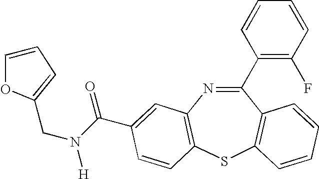 | 7.9 | 100 |
| 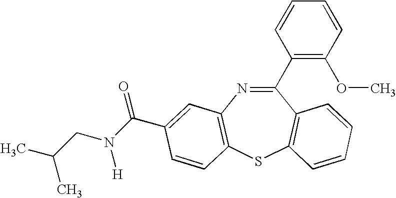 | 6.8 | 91 |
| 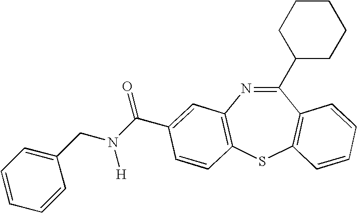 | 7.8 | 97 |
| 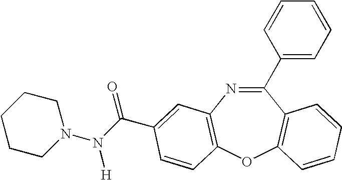 | 6.8 | 57 |
| 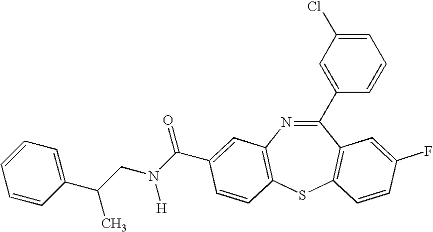 | 7.8 | 113 |
| 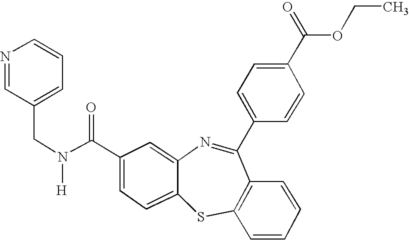 | 6.8 | 73 |
| 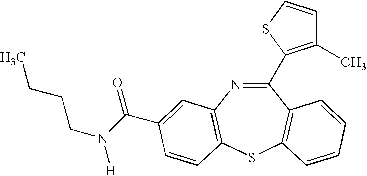 | 7.8 | 91 |
| 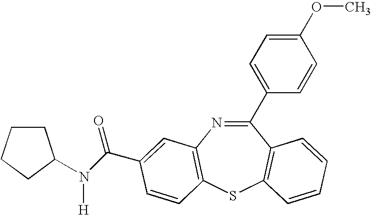 | 6.8 | 103 |
| 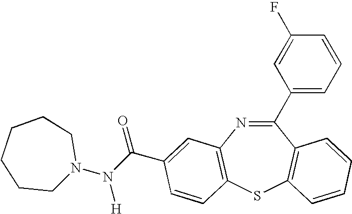 | 7.8 | 96 |
| 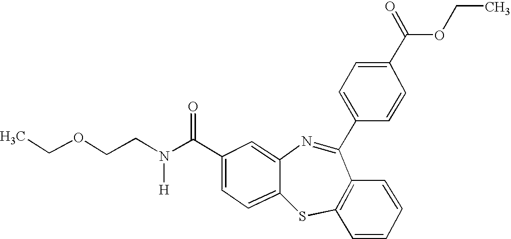 | 6.8 | 77 |
| 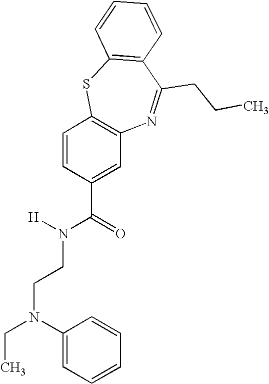 | 7.8 | 106 |
| 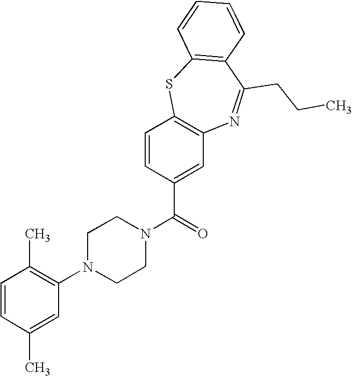 | 6.8 | 90 |
| 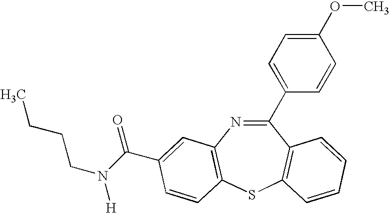 | 7.8 | 112 |
| 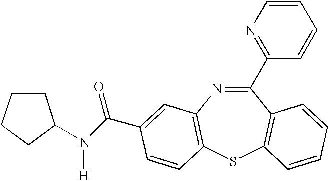 | 6.8 | 81 |
| 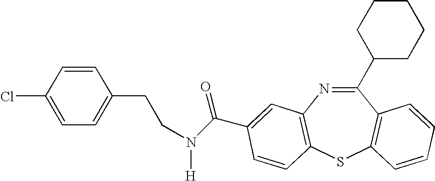 | 7.8 | 104 |
| 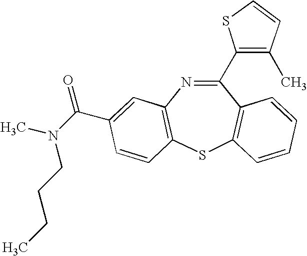 | 6.8 | 72 |
| 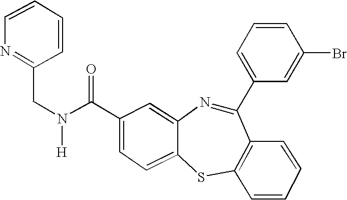 | 7.8 | 85 |
| 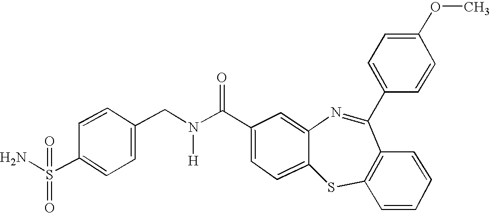 | 6.8 | 84 |
| 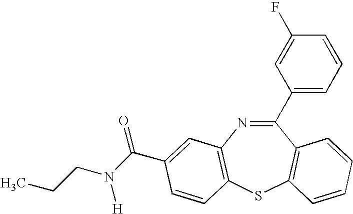 | 7.8 | 109 |
| 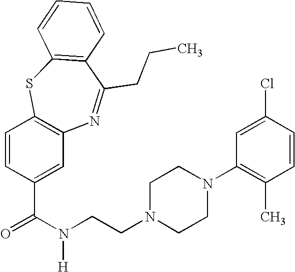 | 6.8 | 113 |
| 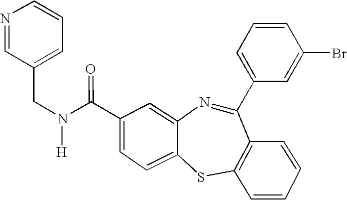 | 7.8 | 105 |
| 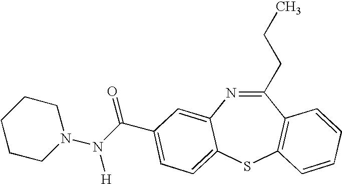 | 6.8 | 80 |
| 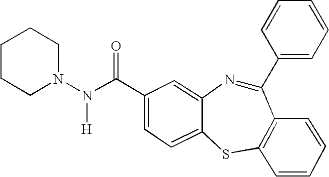 | 7.8 | 115 |
| 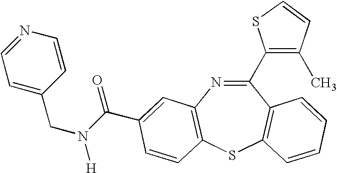 | 6.8 | 92 |
| 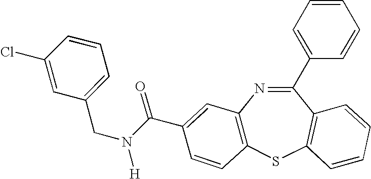 | 7.8 | 107 |
| 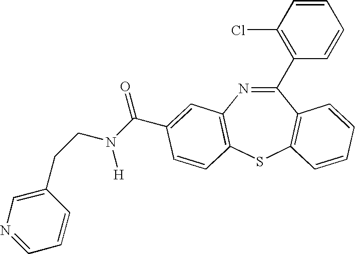 | 6.8 | 76 |
| 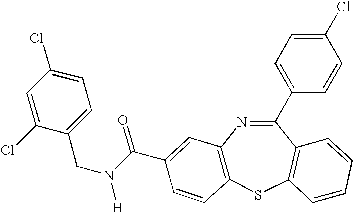 | 7.8 | 101 |
| 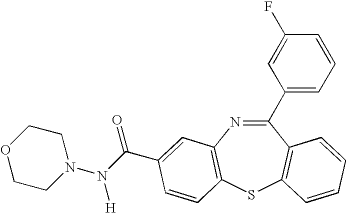 | 6.7 | 63 |
| 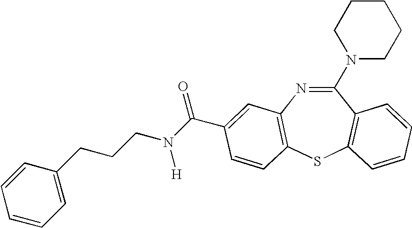 | 7.8 | 104 |
| 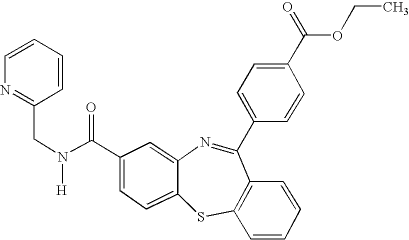 | 6.7 | 100 |
| 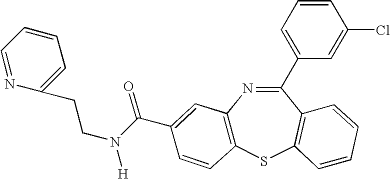 | 7.8 | 103 |
| 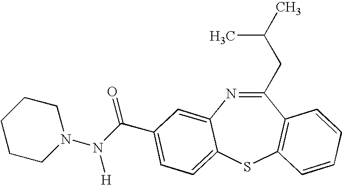 | 6.7 | 90 |
| 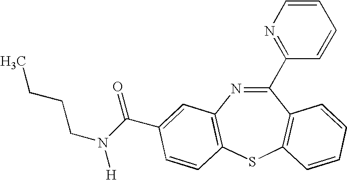 | 7.8 | 100 |
| 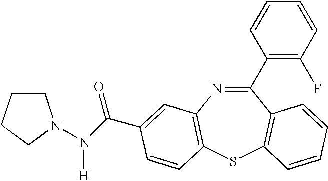 | 6.7 | 69 |
| 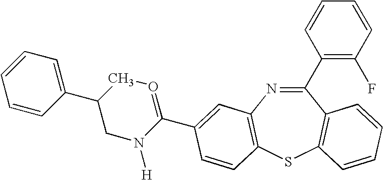 | 7.8 | 110 |
| 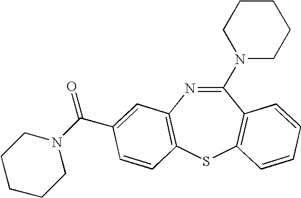 | 6.7 | 89 |
| 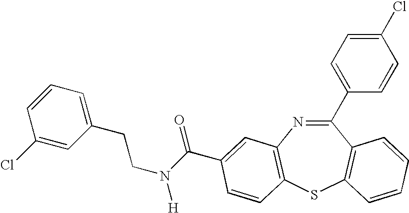 | 7.8 | 99 |
| 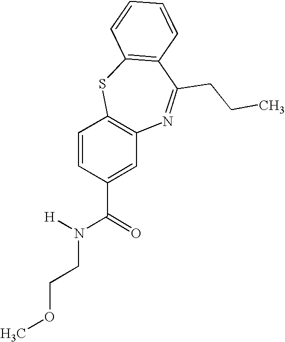 | 6.7 | 107 |
| 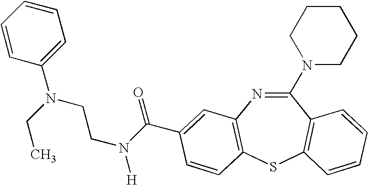 | 7.8 | 105 |
| 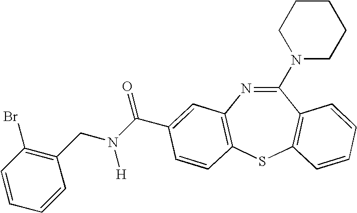 | 6.7 | 62 |
| 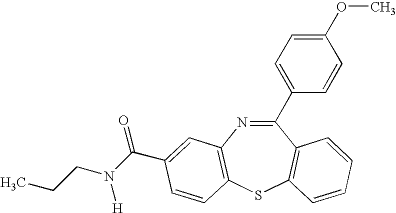 | 7.8 | 112 |
| 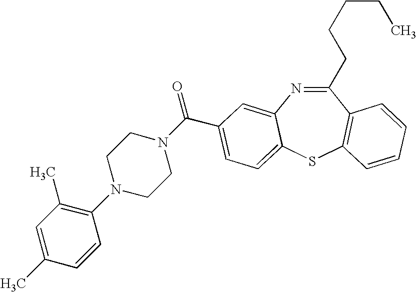 | 6.7 | 107 |
| 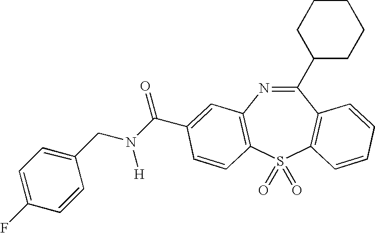 | 7.8 | 120 |
| 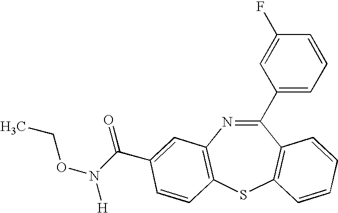 | 6.7 | 81 |
| 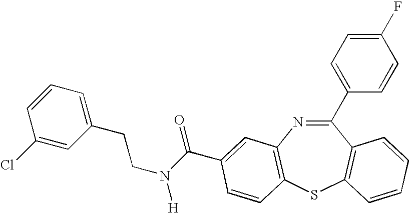 | 7.8 | 98 |
| 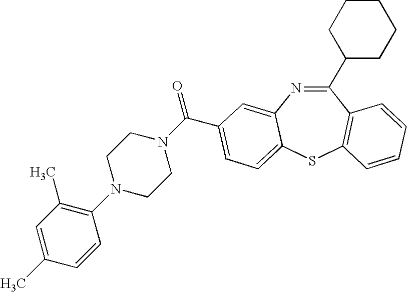 | 6.7 | 103 |
| 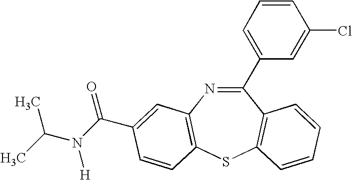 | 7.8 | 118 |
| 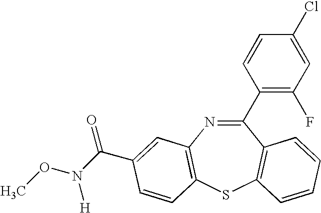 | 6.7 | 69 |
| 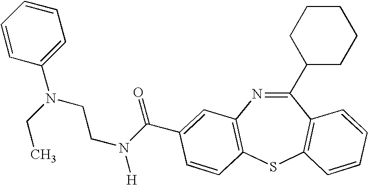 | 7.8 | 105 |
| 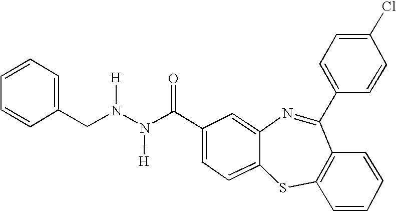 | 6.7 | 66 |
| 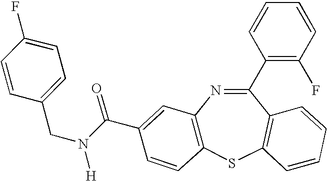 | 7.8 | 102 |
| 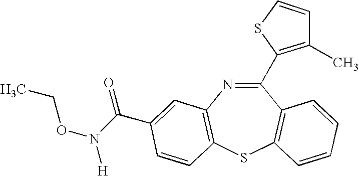 | 6.7 | 77 |
| 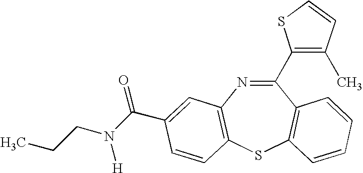 | 7.8 | 99 |
| 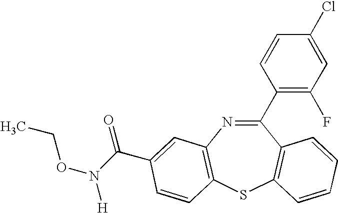 | 6.7 | 87 |
| 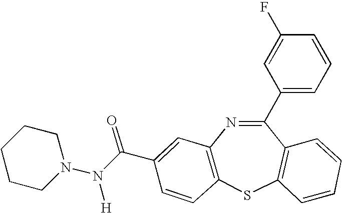 | 7.8 | 111 |
| 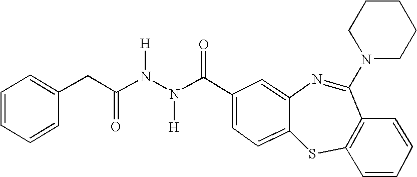 | 6.7 | 69 |
| 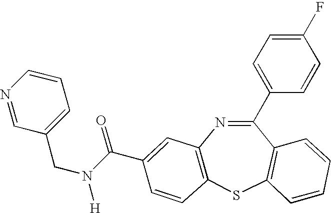 | 7.8 | 123 |
| 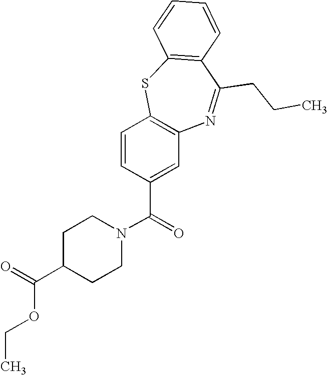 | 6.7 | 108 |
| 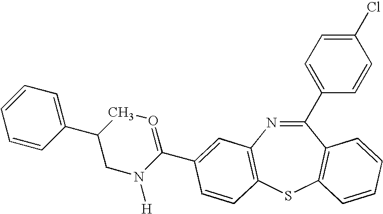 | 7.7 | 106 |
| 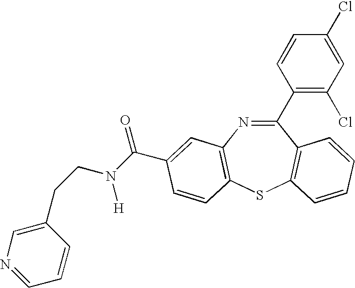 | 6.7 | 74 |
| 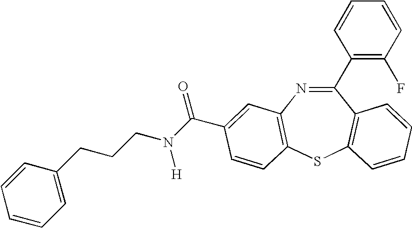 | 7.7 | 106 |
| 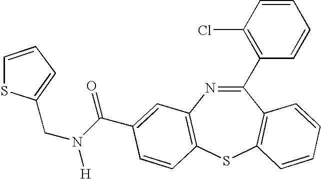 | 6.7 | 87 |
| 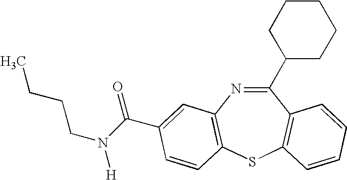 | 7.7 | 106 |
| 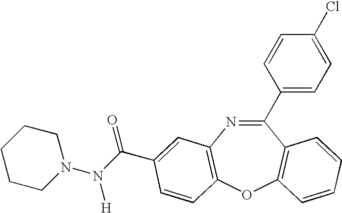 | 6.6 | 51 |
| 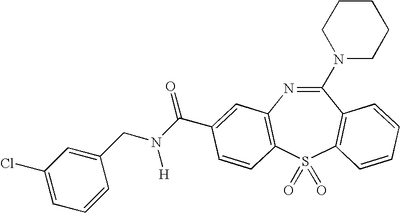 | 7.7 | 91 |
| 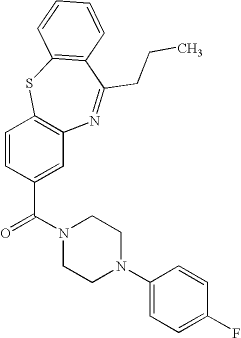 | 6.6 | 108 |
| 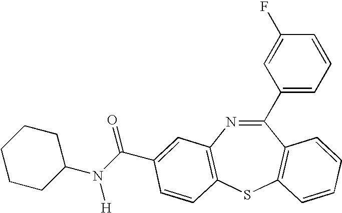 | 7.7 | 127 |
| 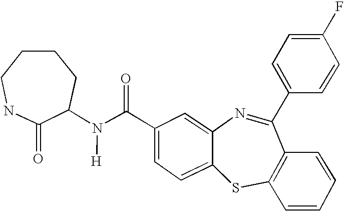 | 6.6 | 83 |
| 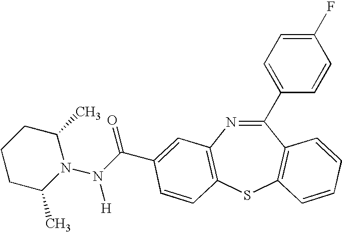 | 7.7 | 115 |
| 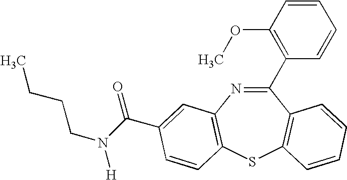 | 6.6 | 80 |
| 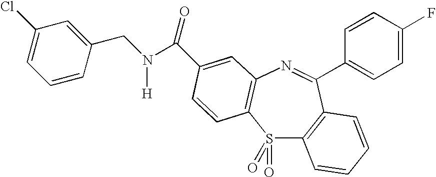 | 7.9 | 108 |
| 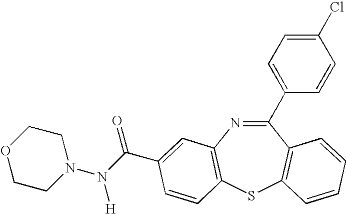 | 6.6 | 75 |
| 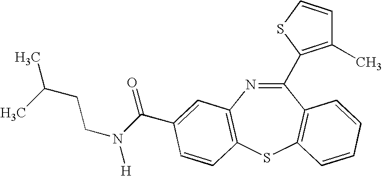 | 7.7 | 106 |
| 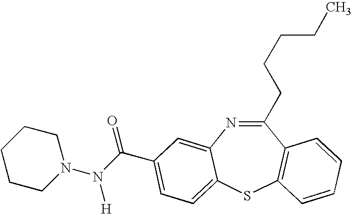 | 6.6 | 100 |
| 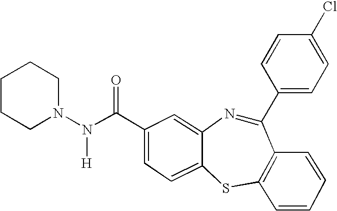 | 7.8 | 103 |
| 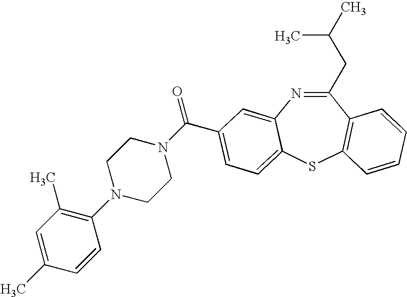 | 6.6 | 105 |
| 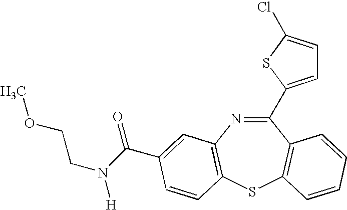 | 7.7 | 95 |
| 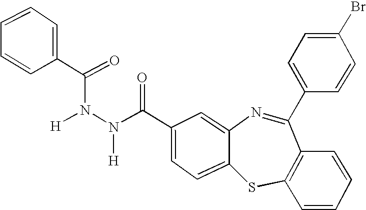 | 6.6 | 88 |
| 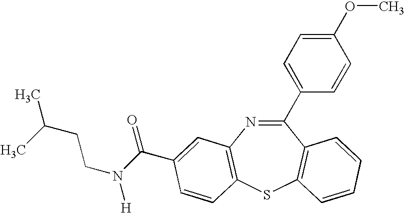 | 7.7 | 104 |
| 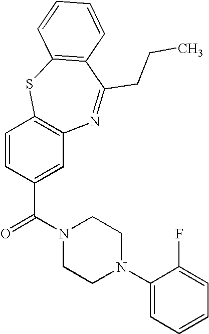 | 6.6 | 97 |
| 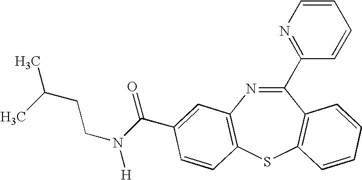 | 7.7 | 114 |
| 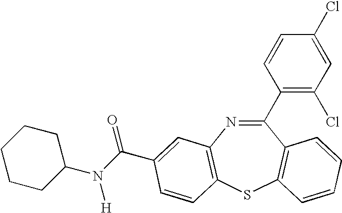 | 6.6 | 83 |
| 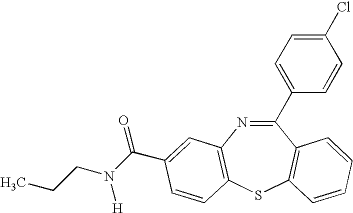 | 7.7 | 105 |
| 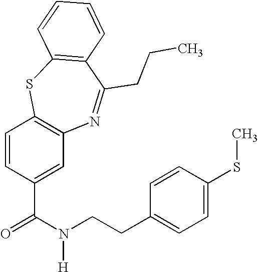 | 6.6 | 103 |
| 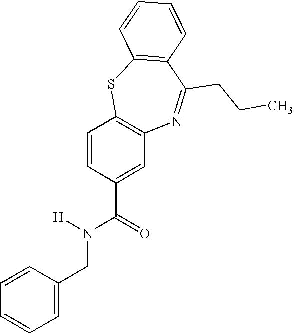 | 7.7 | 111 |
| 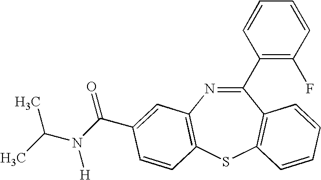 | 6.6 | 82 |
| 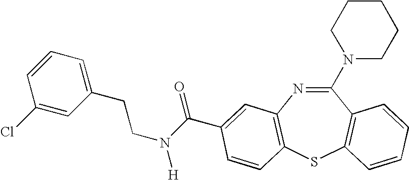 | 7.7 | 108 |
| 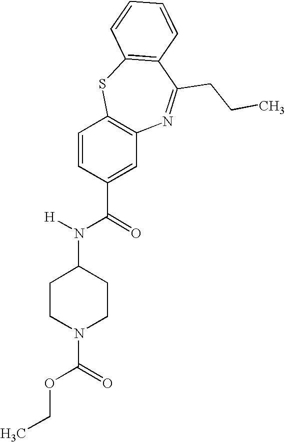 | 6.6 | 77 |
| 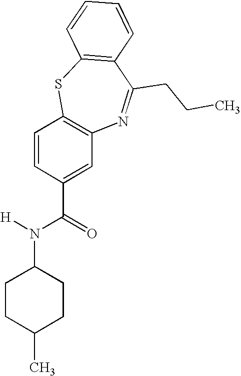 | 7.7 | 114 |
| 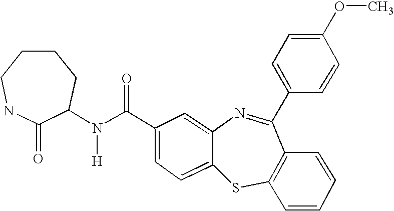 | 6.6 | 62 |
| 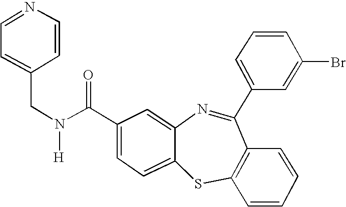 | 7.7 | 95 |
| 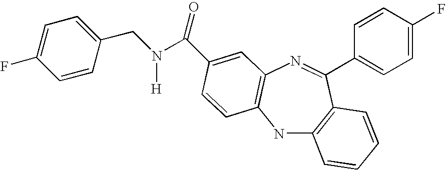 | 6.6 | 61 |
| 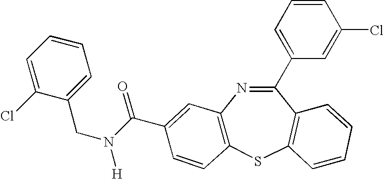 | 7.7 | 102 |
| 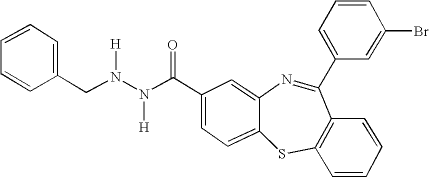 | 6.6 | 86 |
| 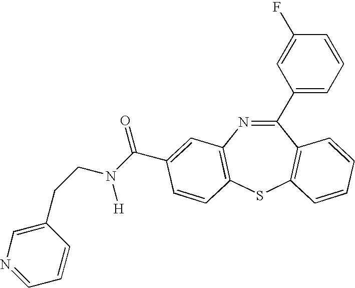 | 7.7 | 94 |
| 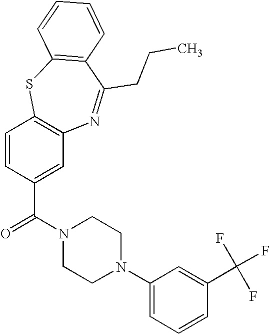 | 6.6 | 91 |
| 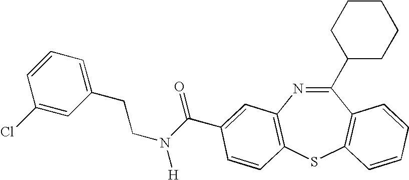 | 7.6 | 102 |
| 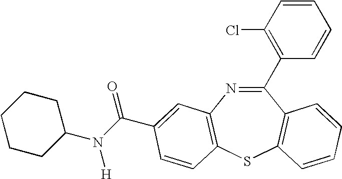 | 6.6 | 67 |
| 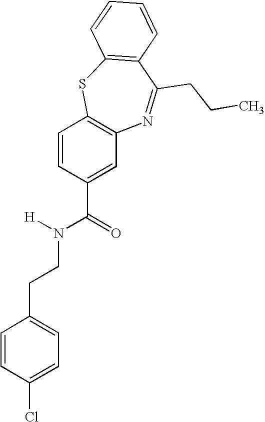 | 7.6 | 110 |
| 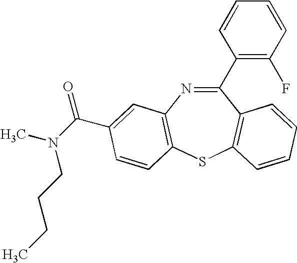 | 6.6 | 77 |
| 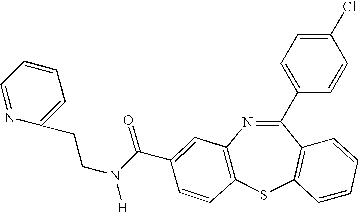 | 7.6 | 92 |
| 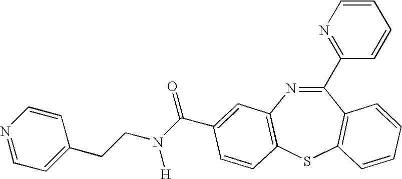 | 6.6 | 51 |
| 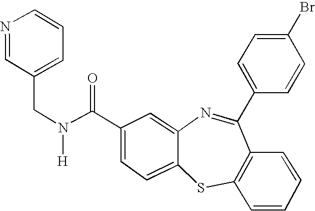 | 7.6 | 106 |
| 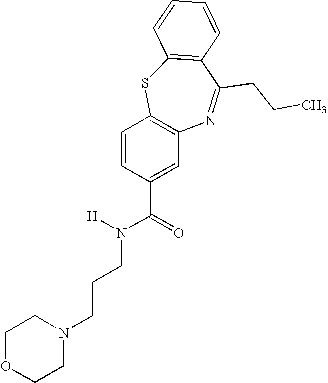 | 6.5 | 98 |
| 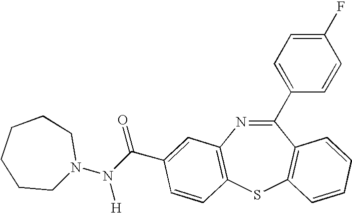 | 7.6 | 92 |
| 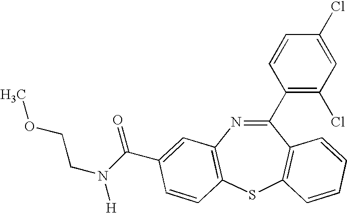 | 6.5 | 69 |
| 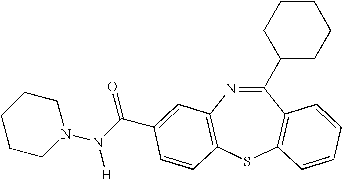 | 7.9 | 100 |
| 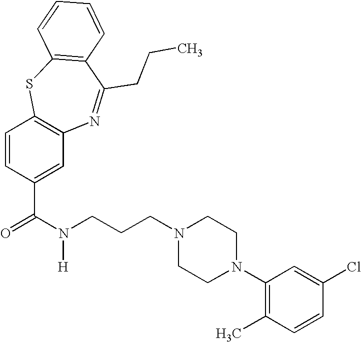 | 6.5 | 117 |
| 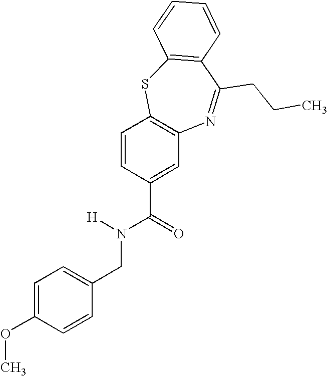 | 7.6 | 96 |
| 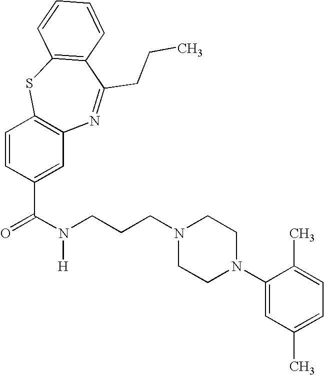 | 6.5 | 126 |
| 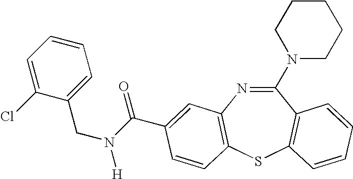 | 7.6 | 117 |
| 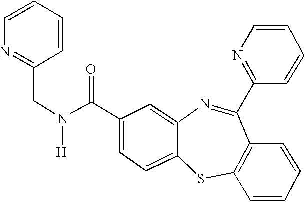 | 6.5 | 81 |
| 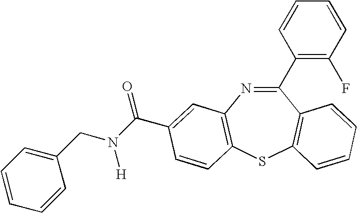 | 7.6 | 96 |
| 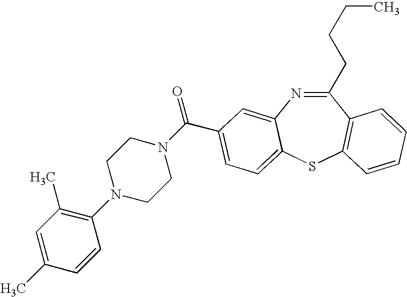 | 6.5 | 104 |
| 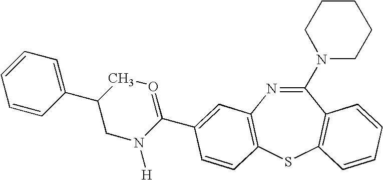 | 7.6 | 105 |
| 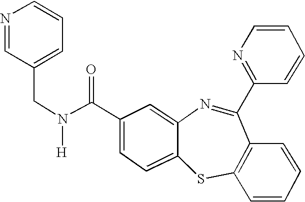 | 6.5 | 67 |
| 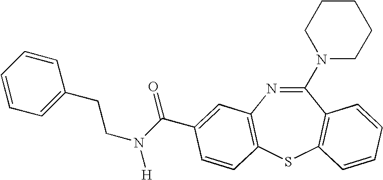 | 7.6 | 106 |
| 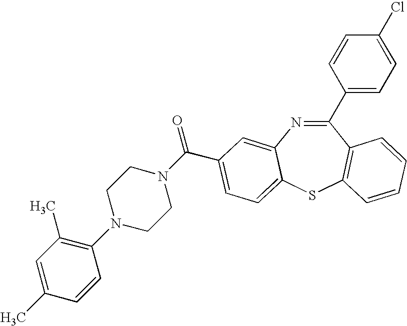 | 6.5 | 94 |
| 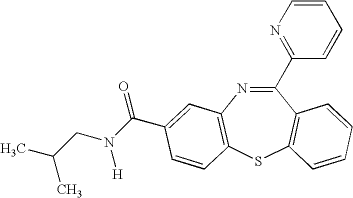 | 7.6 | 96 |
| 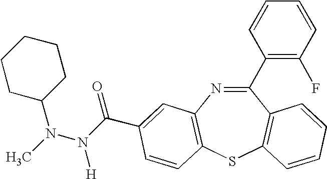 | 6.5 | 74 |
| 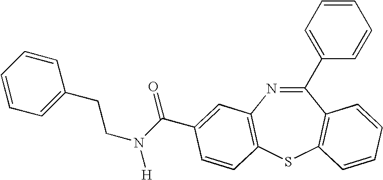 | 7.6 | 106 |
| 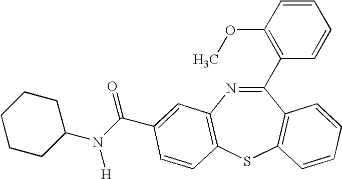 | 6.5 | 72 |
| 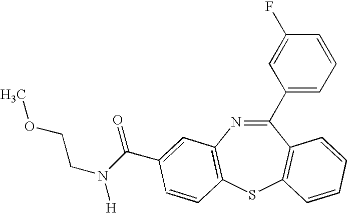 | 7.6 | 115 |
| 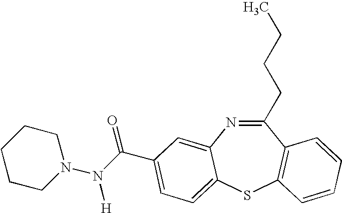 | 6.5 | 82 |
| 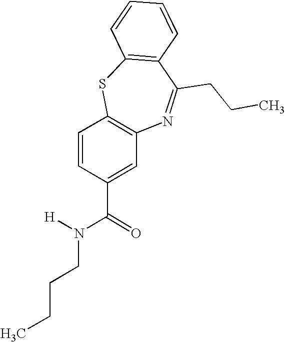 | 7.6 | 111 |
| 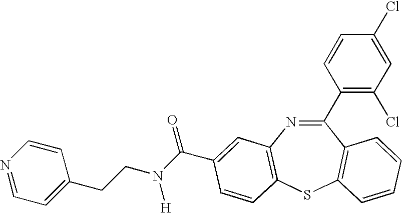 | 6.5 | 72 |
| 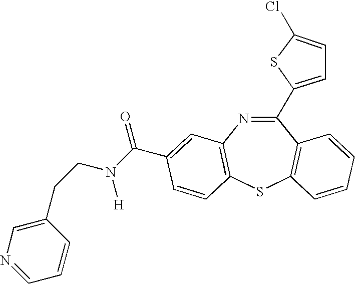 | 7.6 | 110 |
| 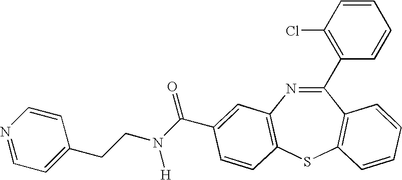 | 6.5 | 82 |
| 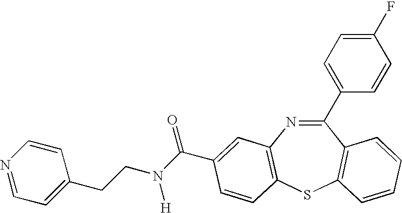 | 7.6 | 94 |
| 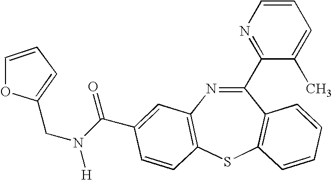 | 6.5 | 83 |
| 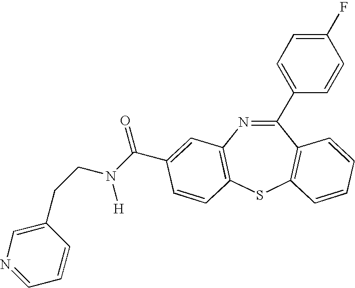 | 7.6 | 119 |
| 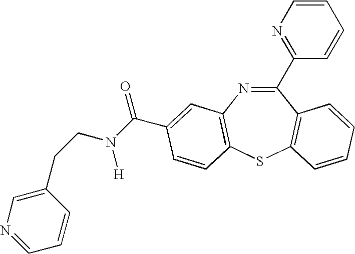 | 6.5 | 77 |
| 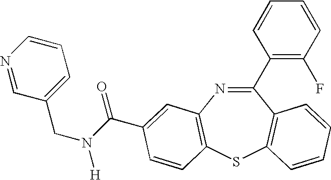 | 7.6 | 114 |
| 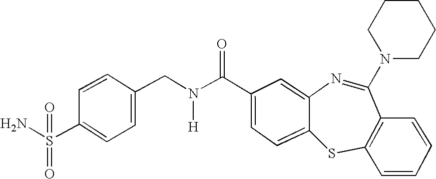 | 6.4 | 78 |
| 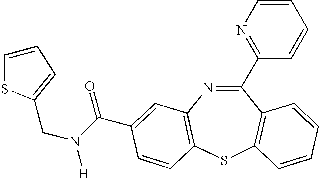 | 7.6 | 118 |
| 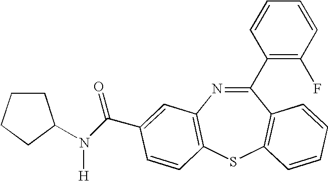 | 6.4 | 79 |
| 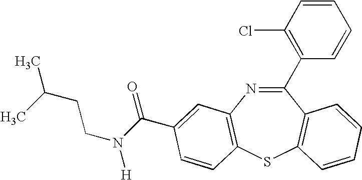 | 7.5 | 105 |
| 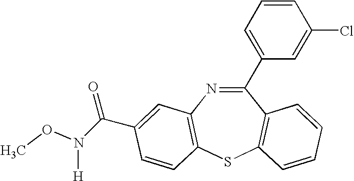 | 6.4 | 89 |
| 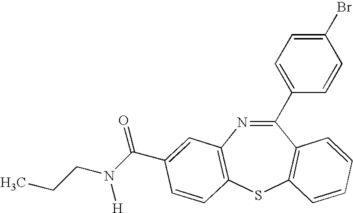 | 7.5 | 92 |
| 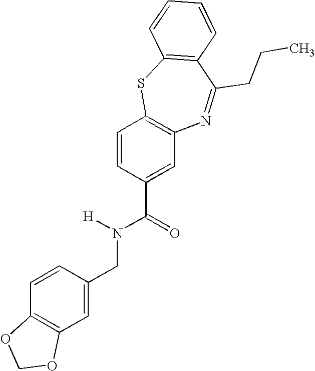 | 6.4 | 91 |
| 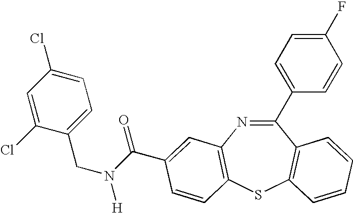 | 7.5 | 96 |
| 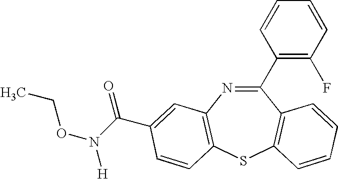 | 6.4 | 73 |
| 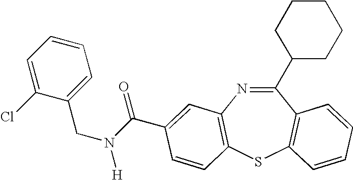 | 7.5 | 91 |
| 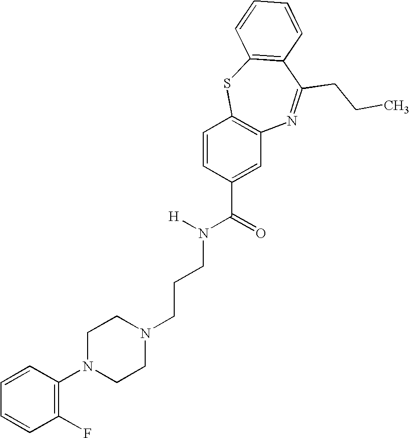 | 6.4 | 120 |
| 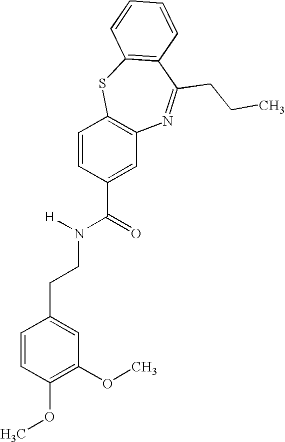 | 7.5 | 105 |
| 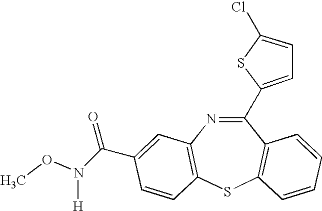 | 6.4 | 66 |
| 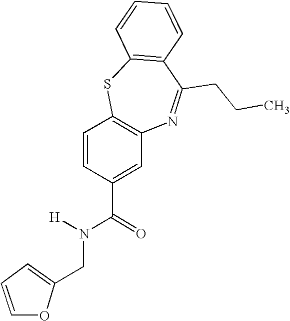 | 7.5 | 103 |
| 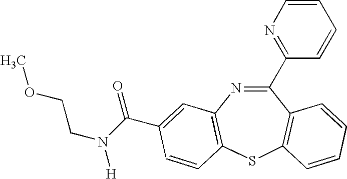 | 6.4 | 71 |
| 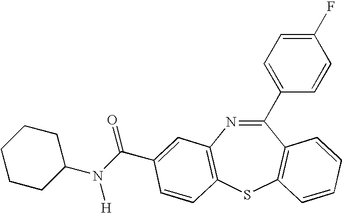 | 7.5 | 111 |
| 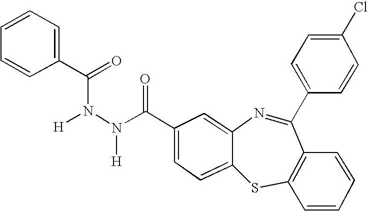 | 6.7 | 71 |
| 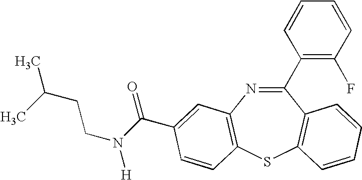 | 7.5 | 117 |
| 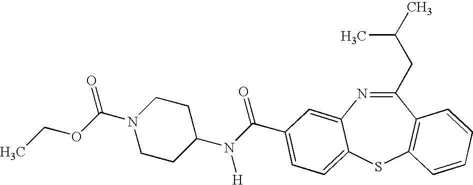 | 6.4 | 89 |
| 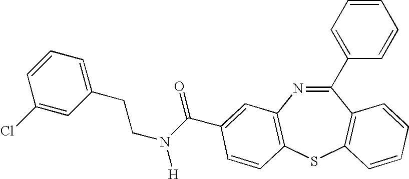 | 7.5 | 102 |
| 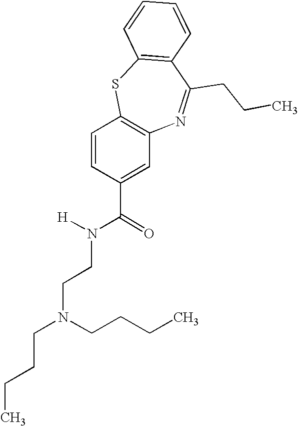 | 6.4 | 109 |
| 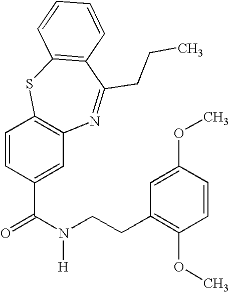 | 7.5 | 98 |
| 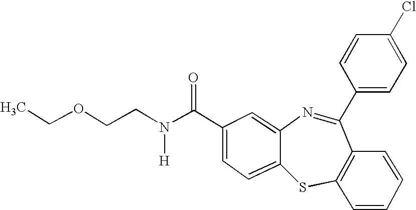 | 6.4 | 1 |
| 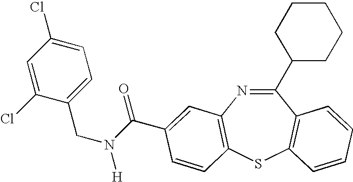 | 7.5 | 90 |
| 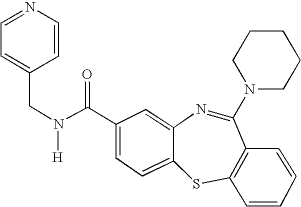 | 6.3 | 81 |
| 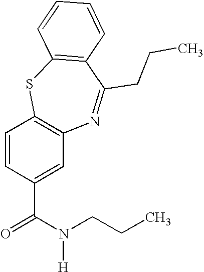 | 7.5 | 95 |
| 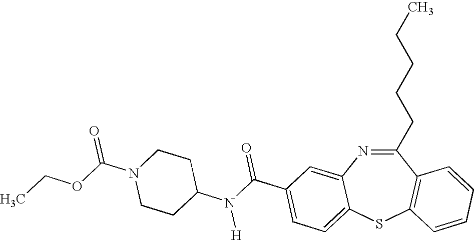 | 6.3 | 87 |
| 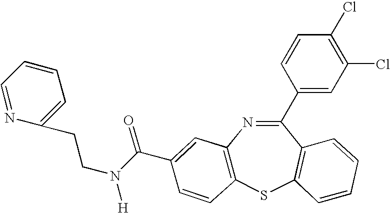 | 7.5 | 93 |
| 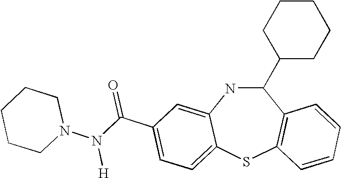 | 6.3 | 43 |
| 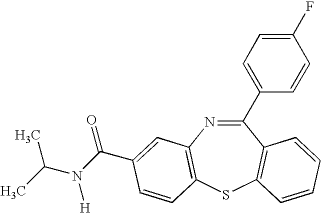 | 7.5 | 97 |
| 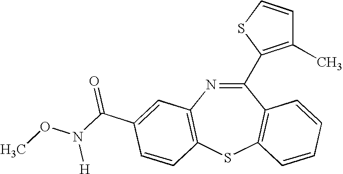 | 6.3 | 45 |
| 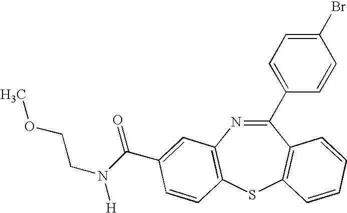 | 7.5 | 69 |
| 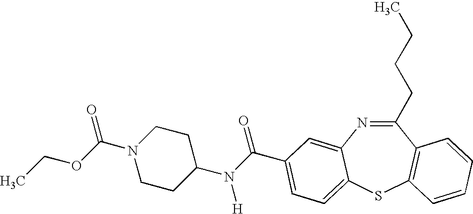 | 6.3 | 96 |
| 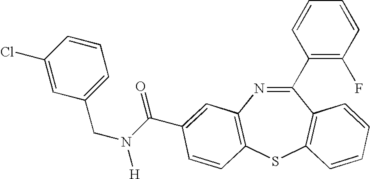 | 7.5 | 99 |
| 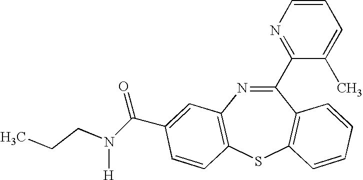 | 6.3 | 63 |
| 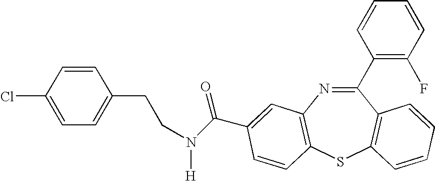 | 7.5 | 101 |
| 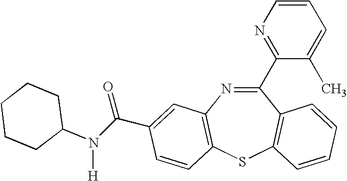 | 6.3 | 61 |
| 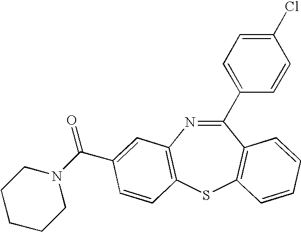 | 7.5 | 95 |
| 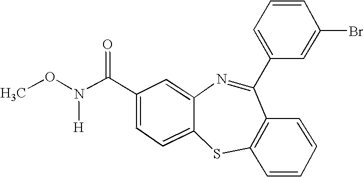 | 6.3 | 73 |
| 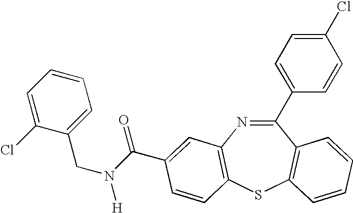 | 7.5 | 95 |
| 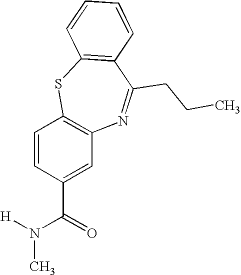 | 6.3 | 119 |
| 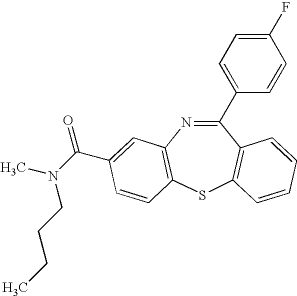 | 7.5 | 107 |
| 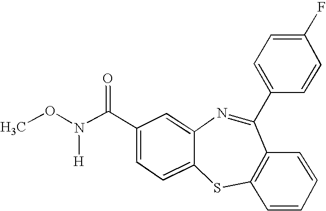 | 6.3 | 65 |
| 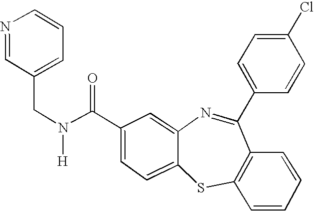 | 7.5 | 88 |
| 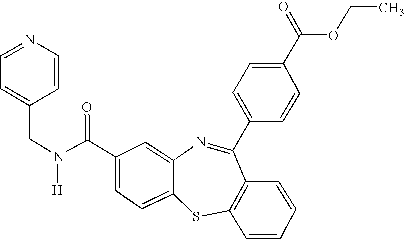 | 6.3 | 69 |
| 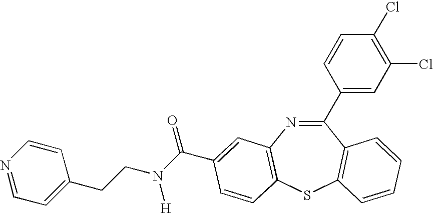 | 7.5 | 119 |
| 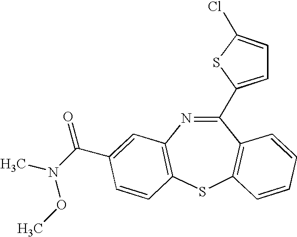 | 6.3 | 55 |
| 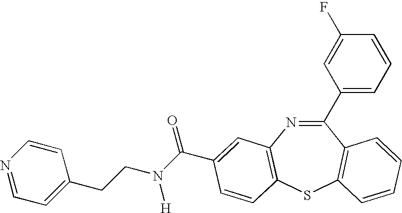 | 7.5 | 106 |
| 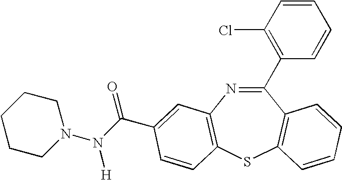 | 6.3 | 45 |
| 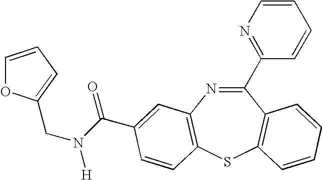 | 7.5 | 102 |
| 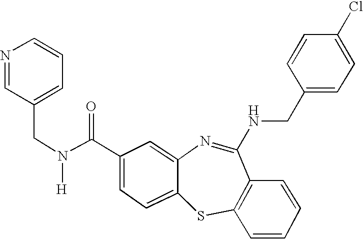 | 6.3 | 76 |
| 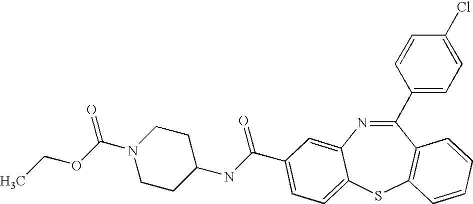 | 7.5 | 109 |
| 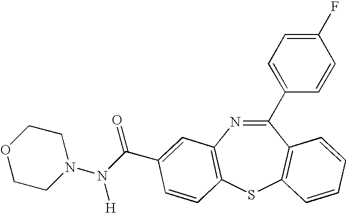 | 6.2 | 77 |
| 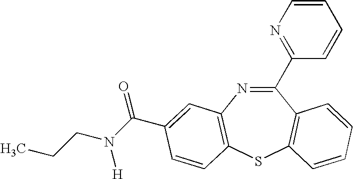 | 7.4 | 116 |
| 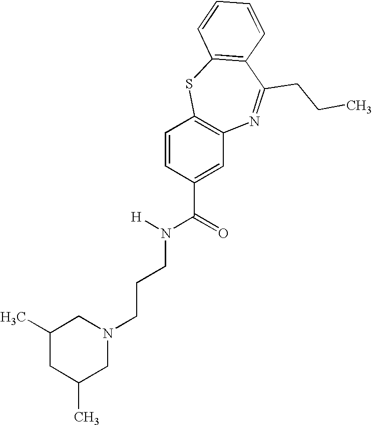 | 6.2 | 136 |
| 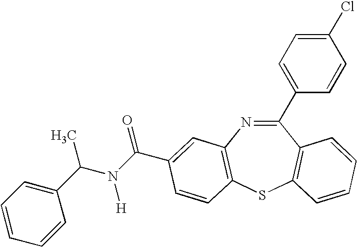 | 7.4 | 88 |
| 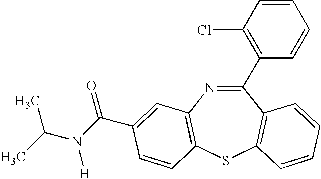 | 6.2 | 46 |
| 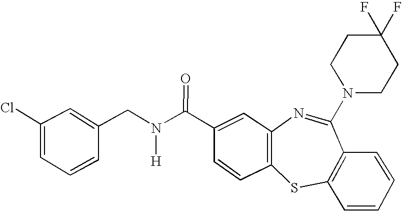 | 7.5 | 100 |
| 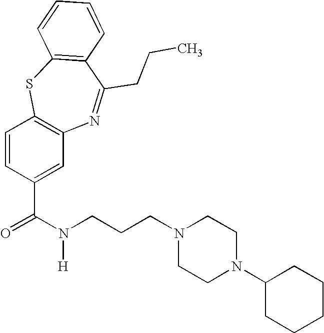 | 6.2 | 118 |
| 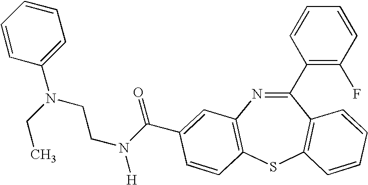 | 7.4 | 100 |
| 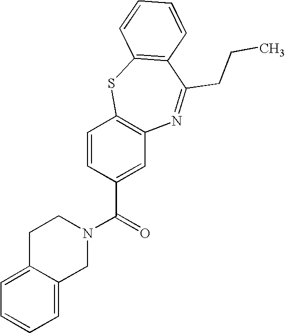 | 6.2 | 95 |
| 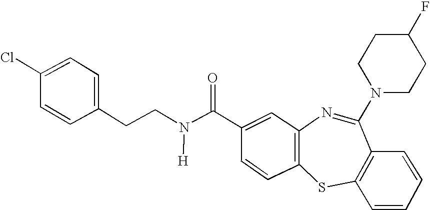 | 7.4 | 103 |
| 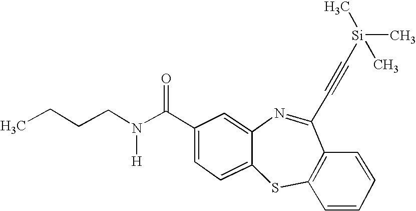 | 6.2 | 64 |
| 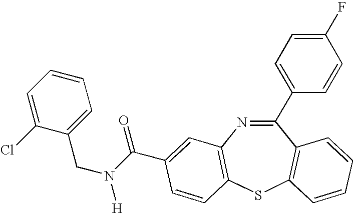 | 7.4 | 89 |
| 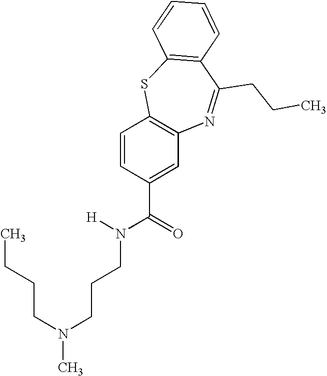 | 6.1 | 124 |
| 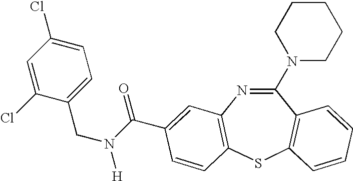 | 7.4 | 101 |
| 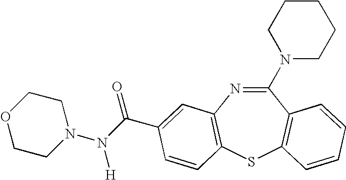 | 6.1 | 99 |
| 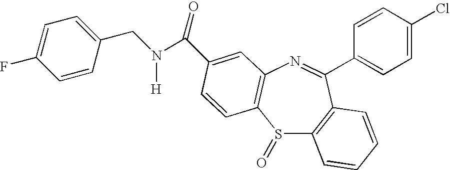 | 7.4 | 98 |
| 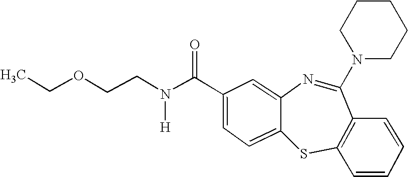 | 6.1 | 103 |
| 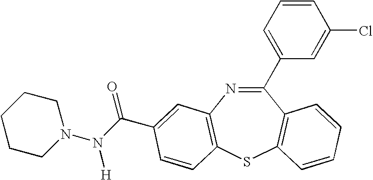 | 7.4 | 106 |
| 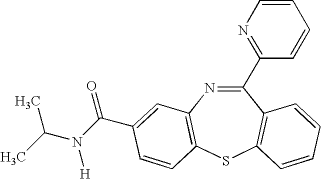 | 6.1 | 60 |
| 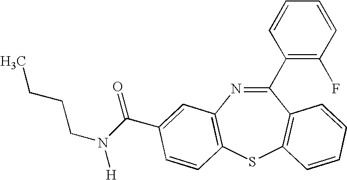 | 7.4 | 96 |
| 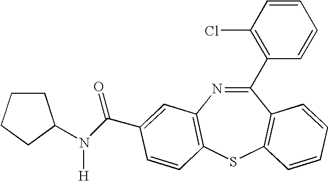 | 6.1 | 59 |
| 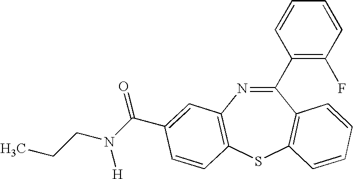 | 7.4 | 97 |
| 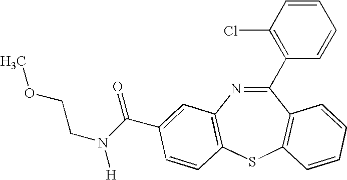 | 6.1 | 62 |
| 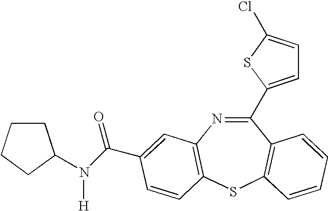 | 7.4 | 86 |
| 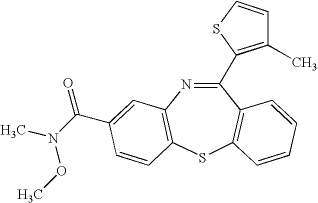 | 6.1 | 42 |
| 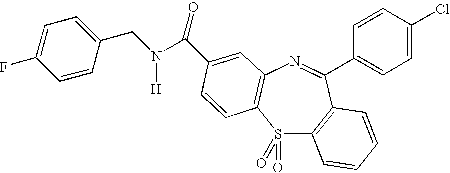 | 7.7 | 108 |
| 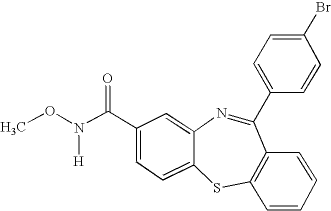 | 6.1 | 68 |
| 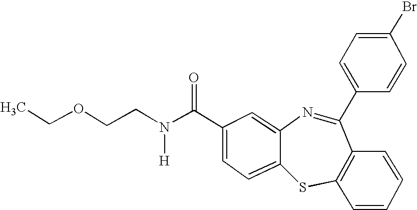 | 7.4 | 85 |
| 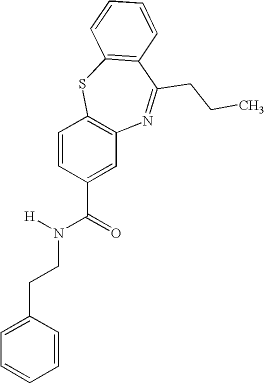 | 6.1 | 98 |
| 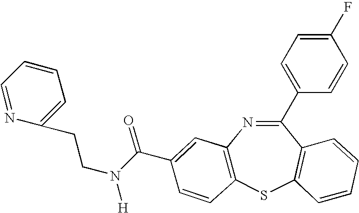 | 7.4 | 101 |
| 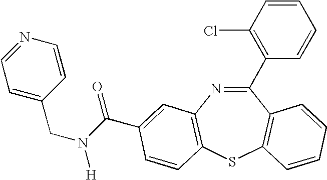 | 6.1 | 67 |
| 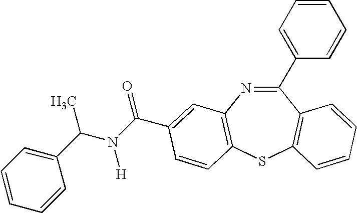 | 7.4 | 85 |
| 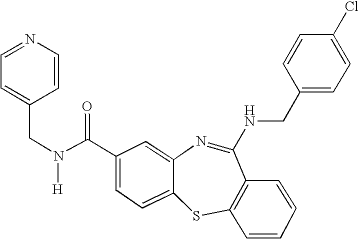 | 6.1 | 56 |
| 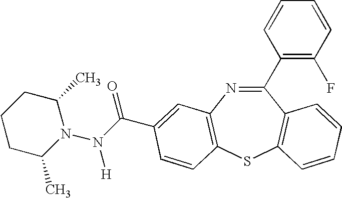 | 7.4 | 103 |
| 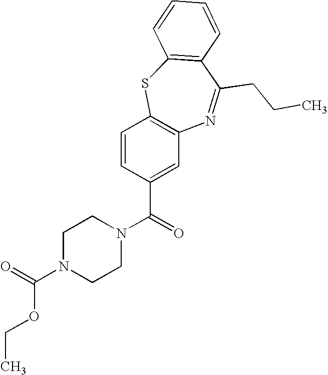 | 6.0 | 77 |
| 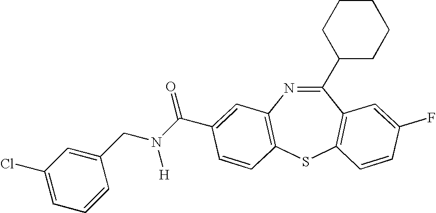 | 7.4 | 100 |
| 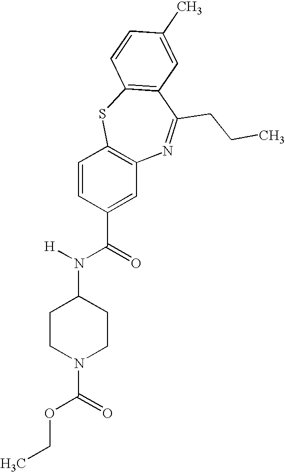 | 6.0 | 79 |
| 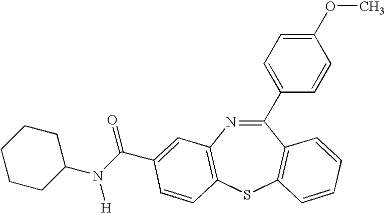 | 7.4 | 115 |
| 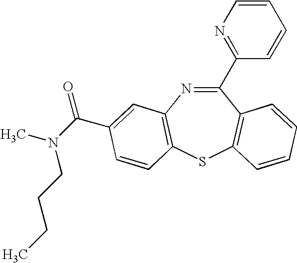 | 6.0 | 57 |
| 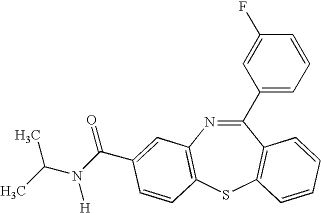 | 7.4 | 104 |
| 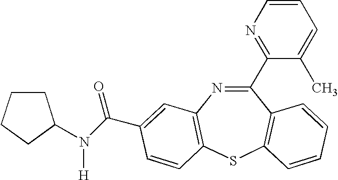 | 6.0 | 57 |
| 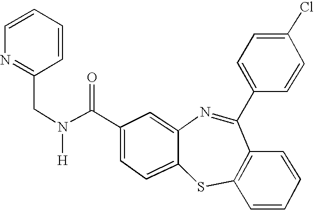 | 7.4 | 125 |
| 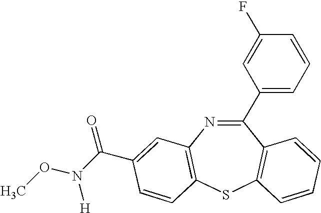 | 6.0 | 67 |
| 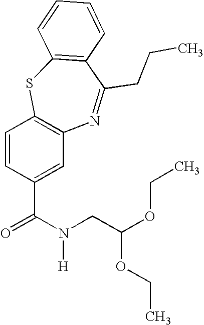 | 7.4 | 107 |
| 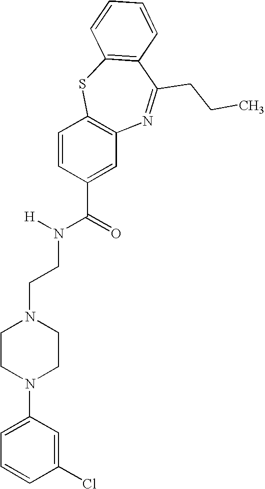 | 5.9 | 76 |
| 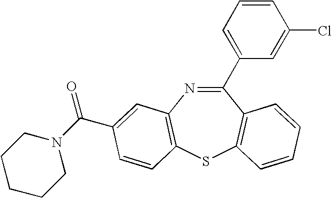 | 7.4 | 80 |
| 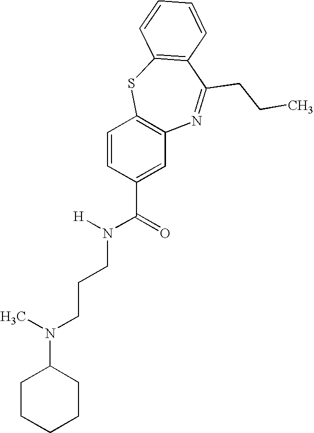 | 5.9 | 120 |
| 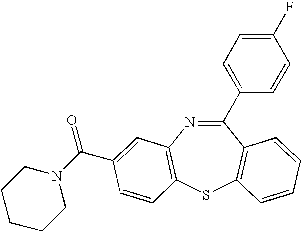 | 7.4 | 90 |
| 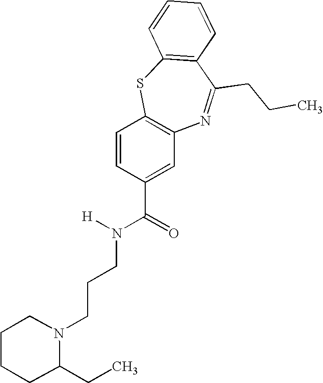 | 5.9 | 128 |
| 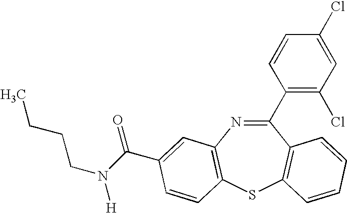 | 7.4 | 98 |
| 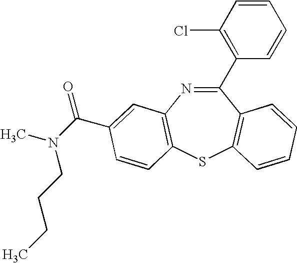 | 5.9 | 40 |
| 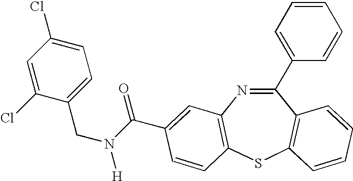 | 7.4 | 91 |
| 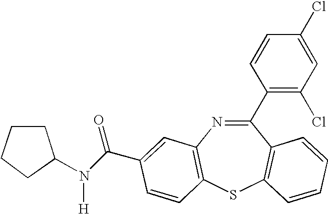 | 5.9 | 52 |
| 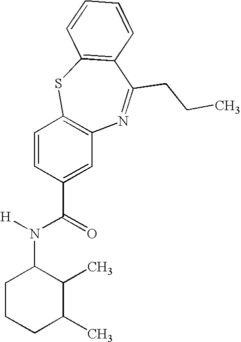 | 7.4 | 101 |
| 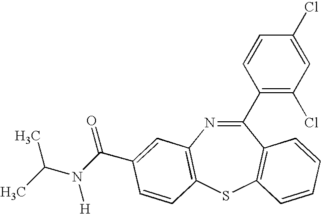 | 5.9 | 45 |
| 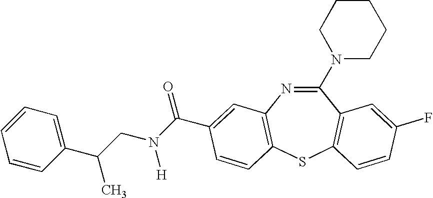 | 7.4 | 102 |
| 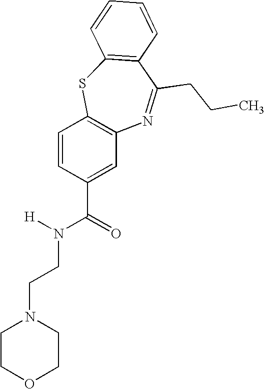 | 5.9 | 98 |
| 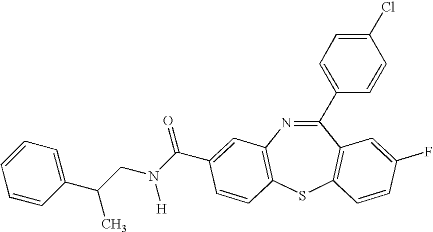 | 7.4 | 110 |
| 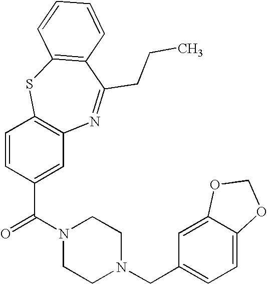 | 5.9 | 96 |
| 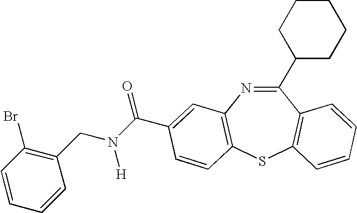 | 7.4 | 85 |
| 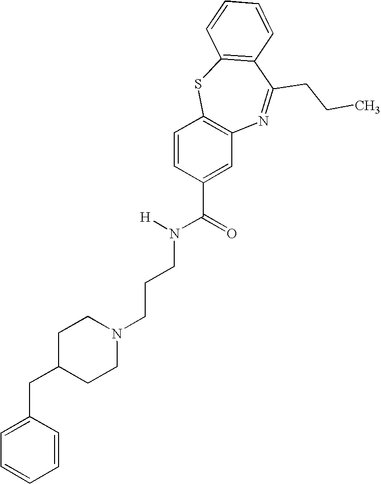 | 5.9 | 115 |
| 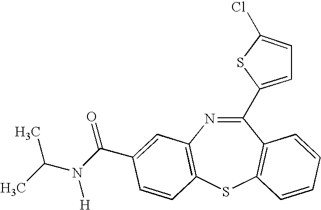 | 7.4 | 78 |
| 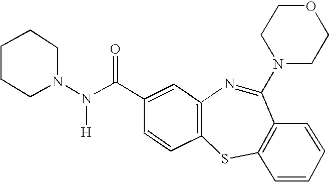 | 5.9 | 76 |
| 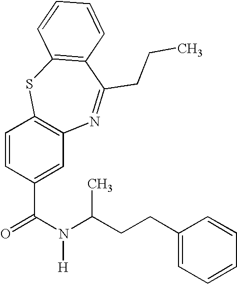 | 7.4 | 95 |
| 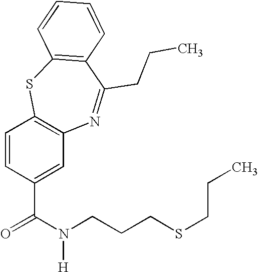 | 5.8 | 87 |
| 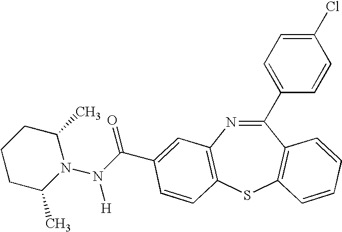 | 7.4 | 96 |
| 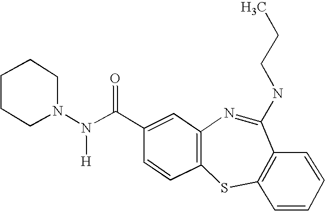 | 5.8 | 89 |
| 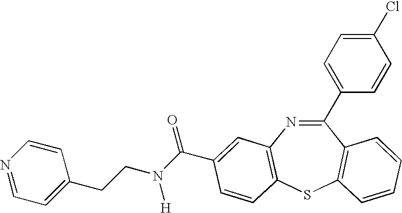 | 7.4 | 116 |
| 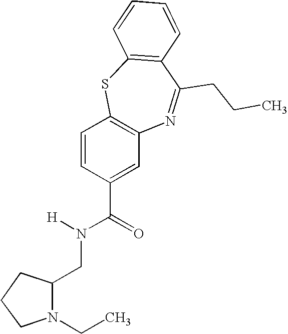 | 5.8 | 133 |
| 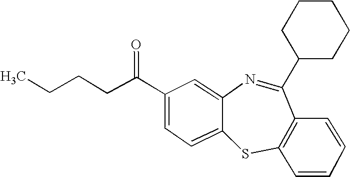 | 7.3 | 37 |
| 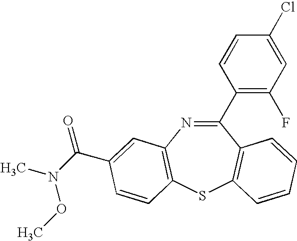 | 5.8 | 46 |
| 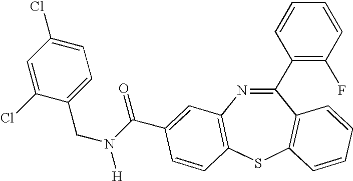 | 7.3 | 85 |
| 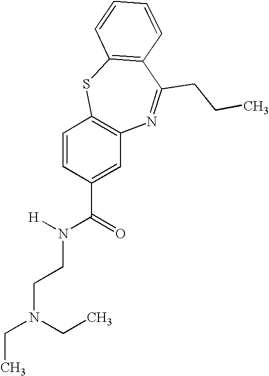 | 5.8 | 131 |
| 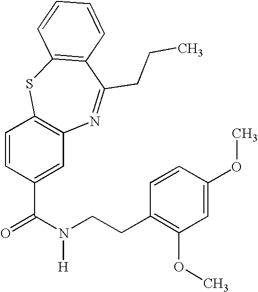 | 7.3 | 101 |
| 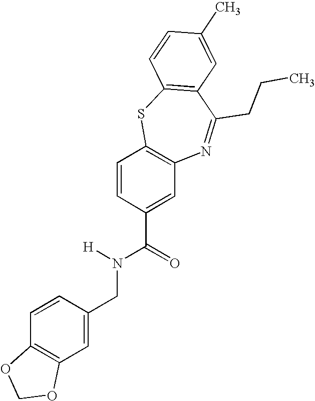 | 5.8 | 77 |
| 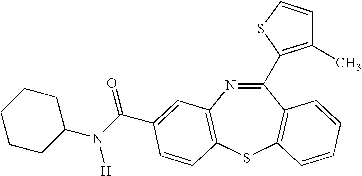 | 7.3 | 88 |
| 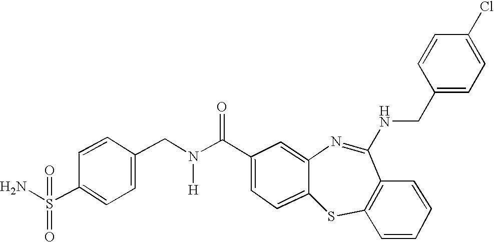 | 5.8 | 41 |
| 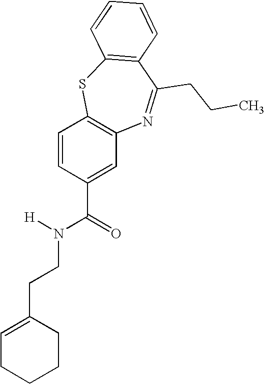 | 7.3 | 118 |
| 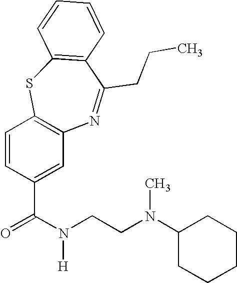 | 5.7 | 123 |
| 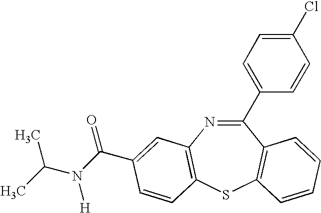 | 7.3 | 98 |
| 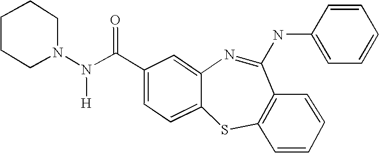 | 5.7 | 92 |
| 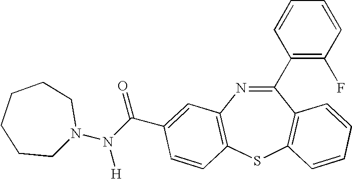 | 7.3 | 87 |
| 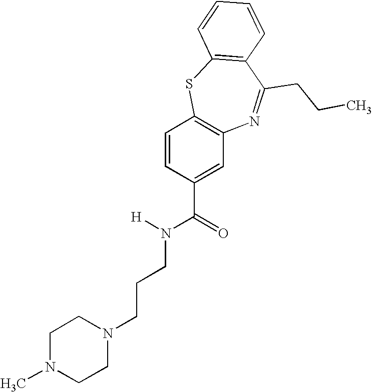 | 5.6 | 120 |
| 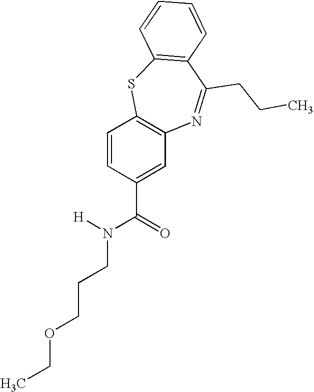 | 7.3 | 92 |
| 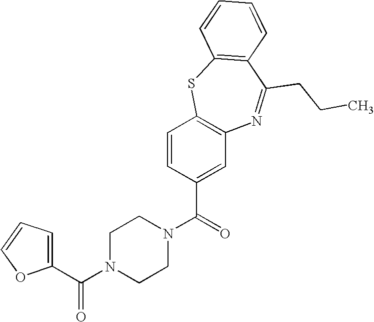 | 5.6 | 88 |
| 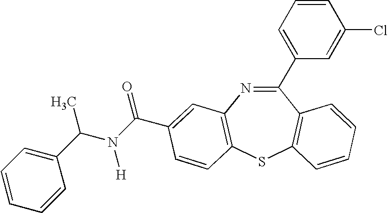 | 7.3 | 87 |
| 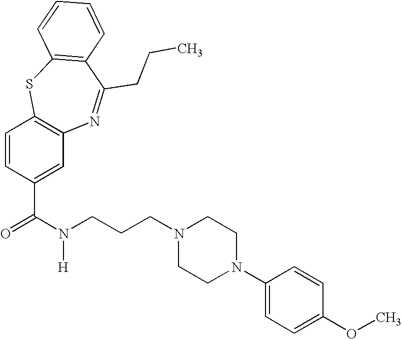 | 5.6 | 79 |
| 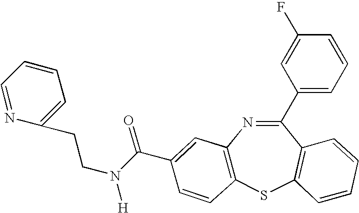 | 7.3 | 91 |
| 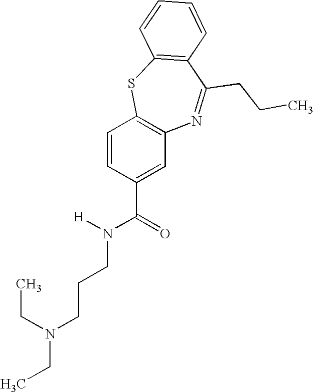 | 5.6 | 125 |
| 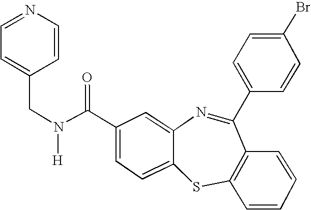 | 7.3 | 84 |
| 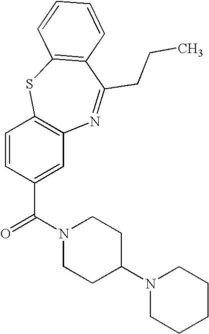 | 5.6 | 128 |
| 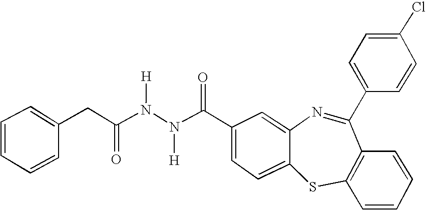 | 7.3 | 85 |
| 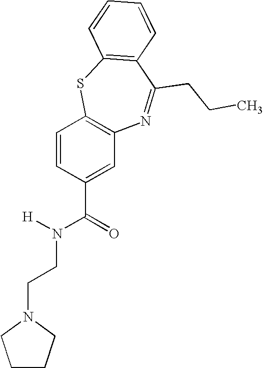 | 5.5 | 138 |
| 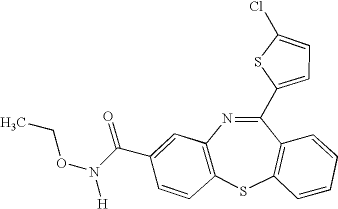 | 7.3 | 97 |
| 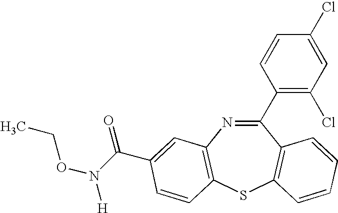 | 5.5 | 37 |
| 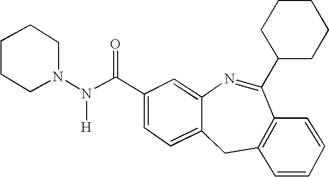 | 7.3 | 87 |
| 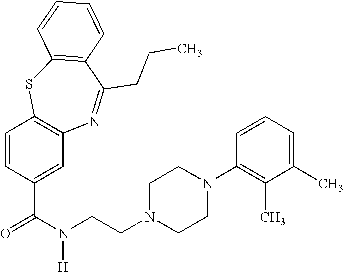 | 5.5 | 87 |
| 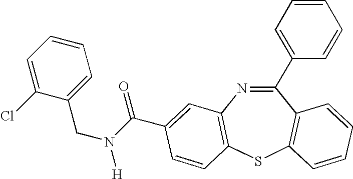 | 7.3 | 86 |
| 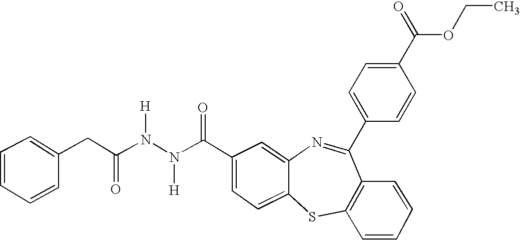 | 5.5 | 61 |
| 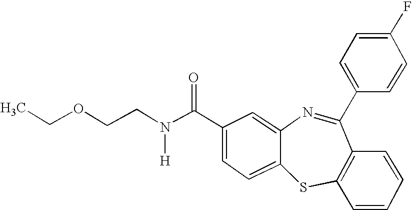 | 7.3 | 94 |
| 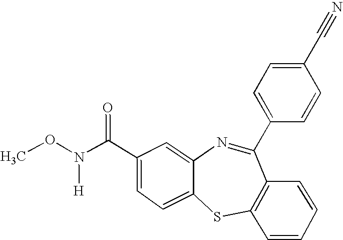 | 1.0 | 36 |
| 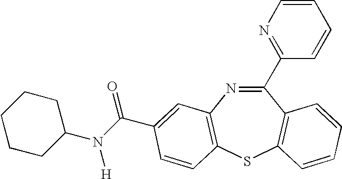 | 7.3 | 112 |
| 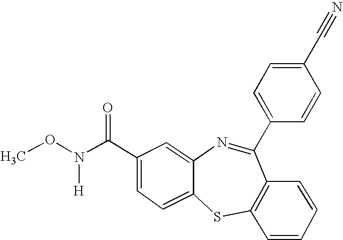 | 1.0 | 3 |
| 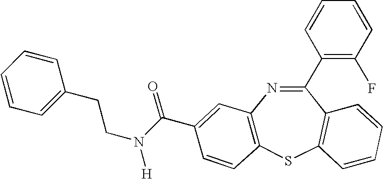 | 7.3 | 98 |
| 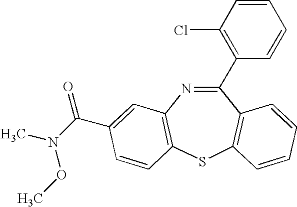 | 1.0 | 10 |
| 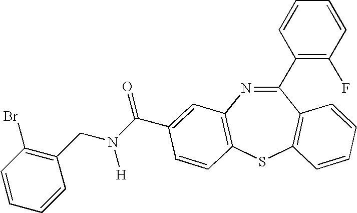 | 7.3 | 65 |
| 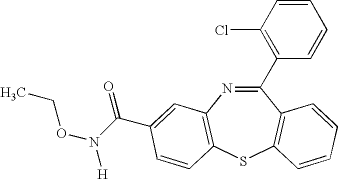 | 1.0 | 19 |
| 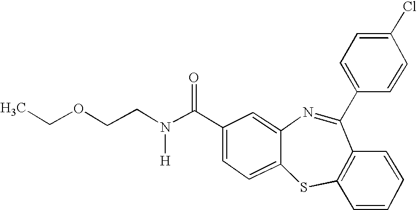 | 7.3 | 92 |
| 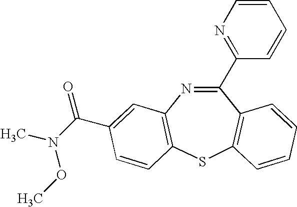 | 1.0 | 8 |
| 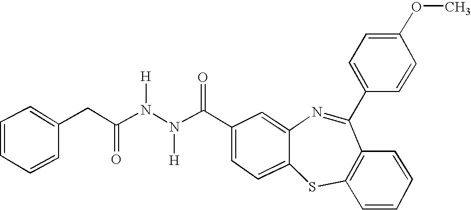 | 7.3 | 77 |
| 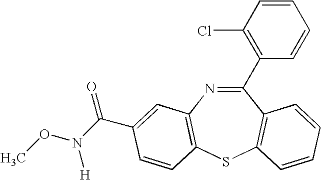 | 1.0 | −15 |
| 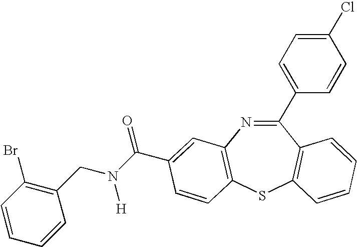 | 7.3 | 94 |
| 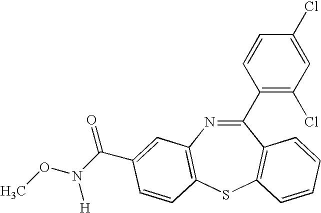 | 1.0 | −27 |
| 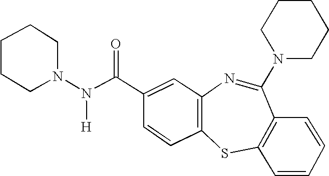 | 7.3 | 104 |
| 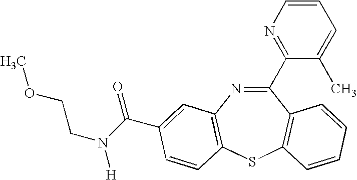 | 1.0 | −6 |
| 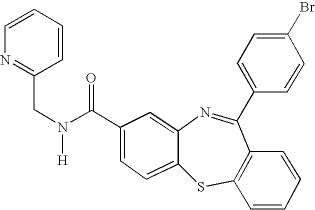 | 7.3 | 90 |
| 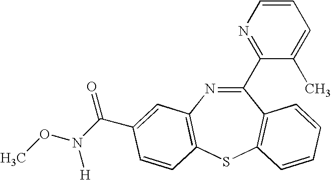 | 1.0 | 8 |
| 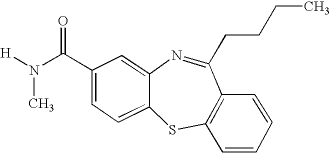 | 7.3 | 55 |
| 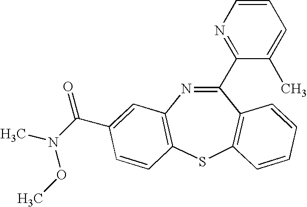 | 1.0 | 0 |
| 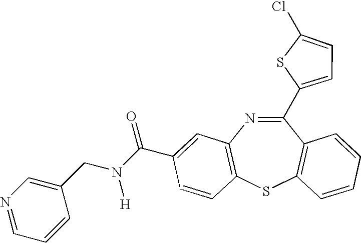 | 7.3 | 99 |
| 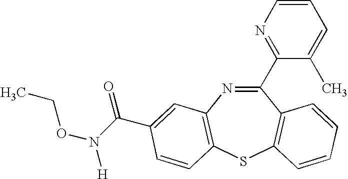 | 1.0 | 21 |
| 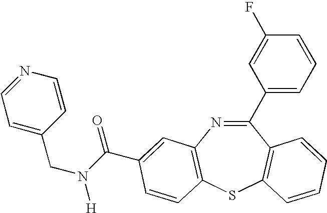 | 7.3 | 95 |
| 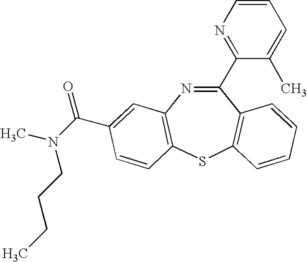 | 1.0 | 14 |
| 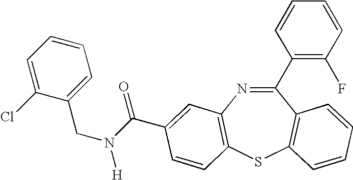 | 7.2 | 72 |
| 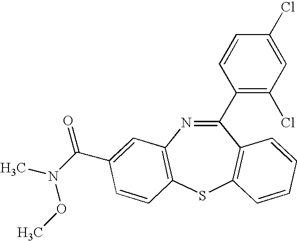 | 1.0 | 18 |
| 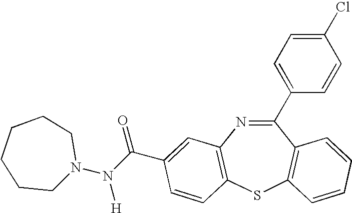 | 7.2 | 103 |
| 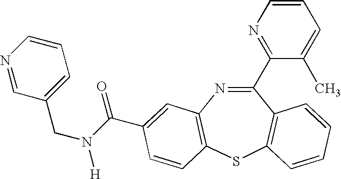 | <6.9 | 39 |
| 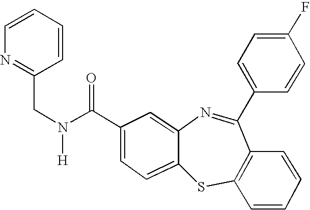 | 7.2 | 90 |
| 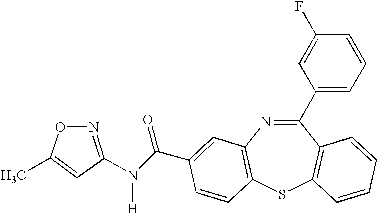 | <5.9 | 29 |
| 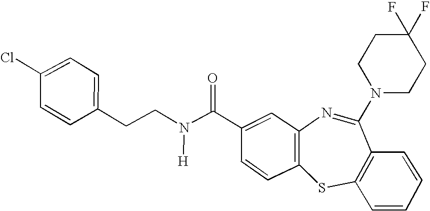 | 7.2 | 99 |
| 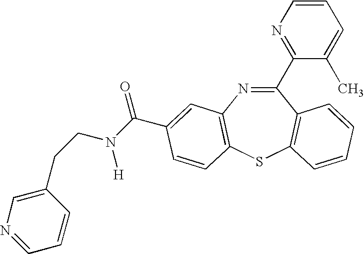 | <5.3 | 28 |

1. A compound of formula (I): 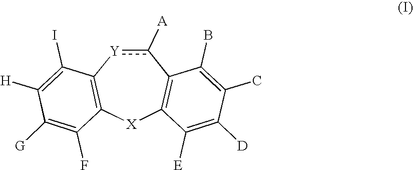
as a single isomer, a mixture of isomers, a racemic mixture of isomers, pharmaceutically acceptable salt, a solvate, metabolite or polymorph thereof, wherein:
X is selected from the group consisting of O, S, S═O, SO₂, NR₁, NC≡N, NC(=Z)R₁, NC(=Z)NR₁ₐR_{1b}, CR₁ₐR_{1b}, C═O, C═CR₁ₐR_{1b}, and SiR₁ₐR_{1b};
Y is —N(R₂) [symbol] or —C(R₁R₂) [symbol] ;
the symbol [symbol] represents a single or double bond, where when [symbol] is a double bond, R₂ is absent;
A is selected from the group consisting of C₃-C₁₂alkyl, C₄-C₁₂alkyl, alkenyl, alkynyl, cycloalkyl, cycloalkenyl, cycloalkynyl, (cycloalkyl)alkyl, (cycloalkenyl)alkyl, (cycloalkynyl)alkyl, aryl, heteroaryl, heteroalicyclyl, aralkyl, heteroaralkyl, (heteroalicyclyl)alkyl, halogen, —NR₁ₐR_{1b}, —N═CR₁ₐR_{1b}, sulfenyl, sulfinyl, sulfonyl, and —(CH₂)₀₋₄—C(=Z)—OR₁, wherein any member of said group can be substituted or unsubstituted;
provided that A cannot be a substituted or unsubstituted piperazine;
B, C, D, E, F, G and I are separately selected from the group consisting of hydrogen, alkyl, alkenyl, alkynyl, cycloalkyl, cycloalkenyl, cycloalkynyl, aryl, heteroaryl, aralkyl, heteroaralkyl, heteroalicyclyl, (heteroalicyclyl)alkyl, halogen, hydroxyl, nitro, sulfenyl, sulfinyl, sulfonyl, haloalkyl, haloalkoxy, —CN, —C(=Z)R₁, —C(=Z)OR₁, —C(=Z)NR₁ₐR_{1b}, —C(=Z)N(R₁)NR₁ₐR_{1b}, —C(=Z)N(R₁)N(R₁)C(=Z)R₁, —C(R₁)═NR₁, —NR₁ₐR_{1b}, —N═CR₁ₐR_{1b}, —N(R₁)—C(═Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(=O)₂R₁, —OR₁, —SR₁, and —OC(=Z)R₁, wherein any member of said group can be substituted or unsubstituted except for hydrogen;
H is selected from the group consisting of alkyl, alkenyl, alkynyl, cycloalkyl, cycloalkenyl, cycloalkynyl, aryl, heteroaryl, aralkyl, heteroaralkyl, heteroalicyclyl, (heteroalicyclyl)alkyl, halogen, hydroxyl, nitro, sulfenyl, sulfinyl, sulfonyl, haloalkyl, haloalkoxy, —CN, —C(=Z)R₁, —C(═Z)OR₁, —C(=Z)NR₁ₐR_{1b}, —C(=Z)N(R₁)NR₁ₐR_{1b}, —C(═Z)N(R₁)N(R₁)C(=Z)R₁, —C(R₁)=NR₁, —NR₁ₐR_{1b}, —N=CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(═O)R₁, —N(R₁)—S(═O)₂R₁, —OR₁, —SR₁, and —OC(=Z)R₁, wherein any member of said group can be substituted or unsubstituted;
with the proviso that H cannot be selected from the group consisting of —CF₃, phenyl, —OS(O)₂—CF₃, methyl, —CN, halogen, and 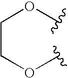
when A is a substituted or unsubstituted heteroalicyclyl containing at least one nitrogen or —NR₁ₐR_{1b};
with the proviso that H cannot be halogen when A is substituted or unsubstituted aryl, substituted or unsubstituted aralkyl, substituted or unsubstituted heteroaryl, halogen, and substituted or unsubstituted sulfenyl; X is —NR₁, wherein R₁ is hydrogen; and Y is —N(R₂) [symbol] , wherein [symbol] is a double bond and R₂ is absent;
Z is O or S;
R₁, R₁ₐ and R_{1b} are each independently selected from the group consisting of: hydrogen, halogen, alkyl, alkenyl, alkynyl, cycloalkyl, cycloalkenyl, cycloalkynyl, aryl, aralkyl, heteroaryl, heteroaralkyl, heteroalicyclyl, (heteroalicyclyl)alkyl, —(CH₂)₀₋₇—OR₃, —(CH₂)₀₋₇—SR₃, —(CH₂)₀₋₇—NR₃ₐR_{3b}, haloalkyl, —C(=Z)R₃, —C(=Z)OR₃and —C(=Z)NR₃ₐR_{3b}, wherein any member of said group can be substituted or unsubstituted except for hydrogen;
or R₁ₐ and R_{1b} can be taken together to form an unsubstituted or substituted heteroalicyclyl having 2 to 9 carbon atoms or an unsubstituted or substituted carbocyclyl having 3 to 9 carbon atoms;
R₂ is absent or is selected from the group consisting of: hydrogen, alkyl, alkenylt, alkynyl, cycloalkyl, cycloalkenyl, cycloalkynyl, aryl, heteroaryl, and heteroalicyclyl, wherein any member of said group can be substituted or unsubstituted except for hydrogen;
R₃, R₃ₐ, and R_{3b} are each independently selected from the group consisting of: hydrogen, alkyl, alkenyl, alkynyl, cycloalkyl, cycloalkenyl, aryl, heteroaryl, heteroalicyclyl, aralkyl, heteroaralkyl, and (heteroalicyclyl)alkyl, wherein any member of said group can be substituted or unsubstituted except for hydrogen;
with the proviso when X is O or —NR₁, wherein R₁ is methyl and Y is —N(R₂) [symbol] , wherein [symbol] is a double bond and R₂ is absent then H cannot be —C(=Z)OR₁, wherein R₁ is hydrogen, methyl, or ethyl; and
with the proviso that when A is halogen, Y is —N(R₂) [symbol] , wherein [symbol] is a double bond and R₂ is absent, and X is S then F cannot be —S(O)₂NR₁ₐR_{1b}, wherein R₁ₐ and R₁ b are both hydrogen.

2. The compound of claim 1, wherein the compound of Formula (I) binds to a cannabinoid receptor.

3. The compound of claim 2, wherein the cannabinoid receptor is a CB1 receptor.

4. (canceled)

5. The compound of claim 1, wherein R₁ₐ, and R_{1b} form an unsubstituted or substituted heteroalicyclyl having 2 to 9 carbon atoms selected from the group consisting of: 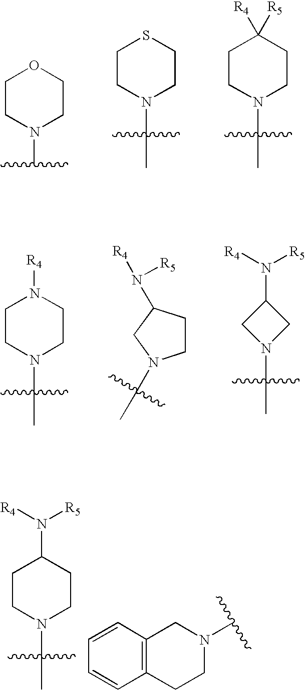
wherein R₄ and R₅ are each independently selected from the group consisting of hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxyl, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino and protected amino.

6. (canceled)

7. The compound of claim 1, wherein X is S, SO, or SO₂.

8. The compound of claim 1, wherein:
H is selected from the group consisting of aryl, heteroaryl, aralkyl, heteroaralkyl, heteroalicyclyl, (heteroalicyclyl)alkyl, halogen, —C(=Z)R₁, —C(=Z)OR₁, —C(Z)NR₁ₐR_{1b}, —C(=Z)N(R₁)NR₁ₐR_{1b}, —C(=Z)N(R₁) N(R₁)N(R₁)C(=Z)R₁, —C(R₁)=NR₁, —NR₁ₐR_{1b}, —N═CR₁ₐR_{1b}, —N(R₁)—C(=Z)R₁, —N(R₁)—C(=Z)NR₁ₐR_{1b}, —S(O)NR₁ₐR_{1b}, —S(O)₂NR₁ₐR_{1b}, —N(R₁)—S(=O)R₁, —N(R₁)—S(═O)₂R₁, and —OC(=Z)R₁, wherein any member of said group can be substituted or unsubstituted.

9. The compound of claim 1, wherein:
H is selected from the group consisting of cycloalkyl, cycloalkenyl, aryl, heteroaryl, aralkyl, heteroaralkyl, heteroalicyclyl, (heteroalicyclyl)alkyl, hydroxyl, sulfenyl, sulfinyl, sulfonyl, haloalkoxy, —C(=Z)OR₁, —C(=Z)N(R₁)NR₁ₐR_{1b}, —C(=Z)N(R₁)N(R₁)C(=Z)R₁, —C(R₁)=NR₁, —NR₁ₐR_{1b}, —N=CR₁ₐR_{1b}, —S(O)NR₁ₐR_{1b}, —N(R₁)—S(═O)R₁, —N(R₁)—S(═O)₂R₁, and —OC(=Z)R₁, wherein any member of said group can be substituted or unsubstituted.

10. (canceled)

11. The compound of claim 9, wherein the unsubstituted or substituted heteroaryl is selected from the group consisting of:

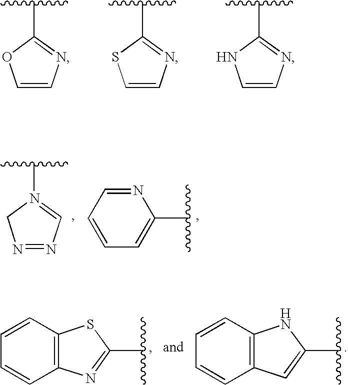

12. (canceled)

13. (canceled)

14. The compound of claim 1, wherein H is —C(=Z)NR₁ₐR_{1b}.

15. The compound of claim 14, wherein R₁ₐ is selected from the group consisting of alkyl, aryl, aralkyl, heteroaryl, heteroaralkyl, heteroalicyclyl, (heteroalicyclyl)alkyl and —(CH₂)₀₋₇—NR₃ₐR_{3b}, wherein any member of said group can be substituted or unsubstituted.

16. The compound of claim 14, wherein R₁ₐ is selected from the group consisting of alkyl, alkoxy, aryl, aralkyl, heteroaryl, and heteroaralkyl, wherein any member of said group can be substituted or unsubstituted.

17. (canceled)

18. The compound of claim 14, wherein R_{1b} is hydrogen or methyl.

19. The compound of claim 15, wherein R₁ is selected from the group consisting of; 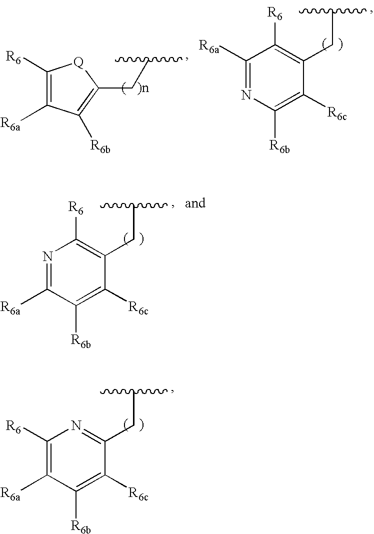
wherein Q is oxygen or sulfur, and n is 1 or 2.

20. (canceled)

21. (canceled)

22. The compound of claim 1, wherein H is —C(=Z)R₁ or —C(=Z)OR₁.

23. The compound of claim 22, wherein H is —C(=Z)R₁ and R₁ is selected from the group consisting of alkyl, cycloalkyl, aralkyl, halogen.

24. The compound of claim 22, wherein H is —C(=Z)OR₁ and R₁ is alkyl or aralkyl.

25. The compound of claim 1, wherein H is —C(=Z)N(R₁)N(R₁)C(=Z)R₁ or —N(R₁)—C(=Z)NR₁ₐR_{1b}.

26. The compound of claim 25, wherein —C(=Z)N(R₁)N(R₁)C(=Z)R₁ is 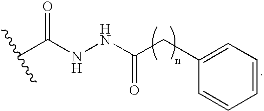
wherein n is 0 or 1.

27. The compound of claim 25, wherein H is —N(R₁)—C(=Z)NR₁ₐR_{1b} and R₁ is hydrogen and R₁ₐ is alkyl or aralkyl.

28. The compound of claim 27, wherein R_{1b} is hydrogen.

29. The compound of claim 1, wherein H is selected from the group consisting of —C(R₁)=NR₁, —N(R₁)—C(=Z)R₁, and —OC(=Z)R₁.

30. The compound of claim 29, wherein H is —C(R₁)—NR₁, —N(R₁)—C(=Z)R₁, and —OC(=Z)R₁ wherein at least on R₁ is hydrogen or alkyl and at least one R₁ is selected from the group consisting of alkyl, aryl, and aralkyl.

31. The compound of claim 1, wherein H is —N(R₁)—S(═O)R₁ or —N(R₁)—S(═O)₂R₁.

32. The compound of claim 31, wherein is —N(R₁)—S(═O)R₁ or —N(R₁)—S(=O)₂R₁ and R₁ is hydrogen, aralkyl, or heteroaryl.

33. The compound of claim 1, wherein H is —S(O)NR₁ₐR_{1b} or —S(O)₂NR₁ₐR_{1b}.

34. The compound of claim 33, wherein H is —S(O)NR₁ₐR_{1b} or —S(O)₂NR₁ₐR_{1b} and R₁ₐ is selected from the group consisting of alkyl, aryl, aralkyl, heteroaryl, and heteroalicyclyl.

35. The compound of claim 33, wherein R_{1b} is hydrogen.

36. The compound of claim 1, wherein if H is —S(O)NR₁ₐR_{1b}, —S(O)₂NR₁ₐR_{1b}, —C(=Z)NR₁ₐR_{1b} or —C(=Z)N(R₁)NR₁ₐR_{1b} then R₁, R₁ₐ and R_{1b} are each independently selected from the group consisting of: 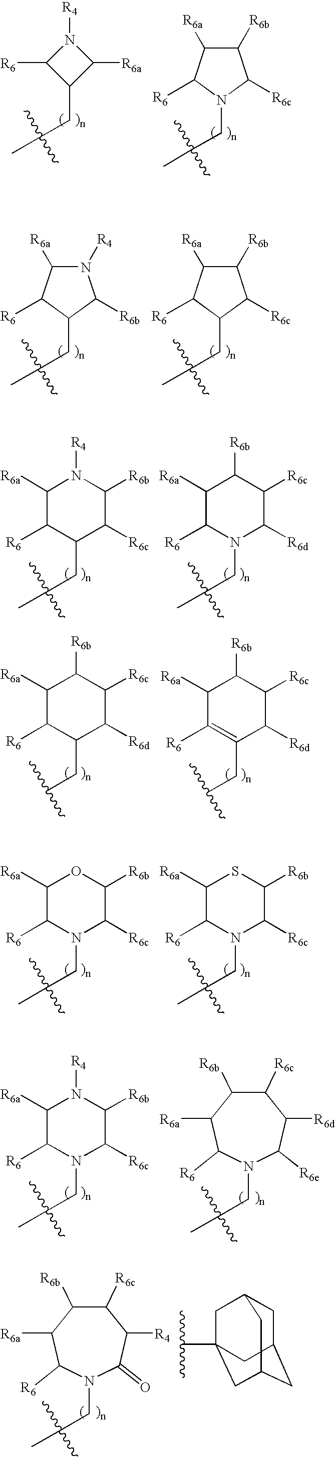 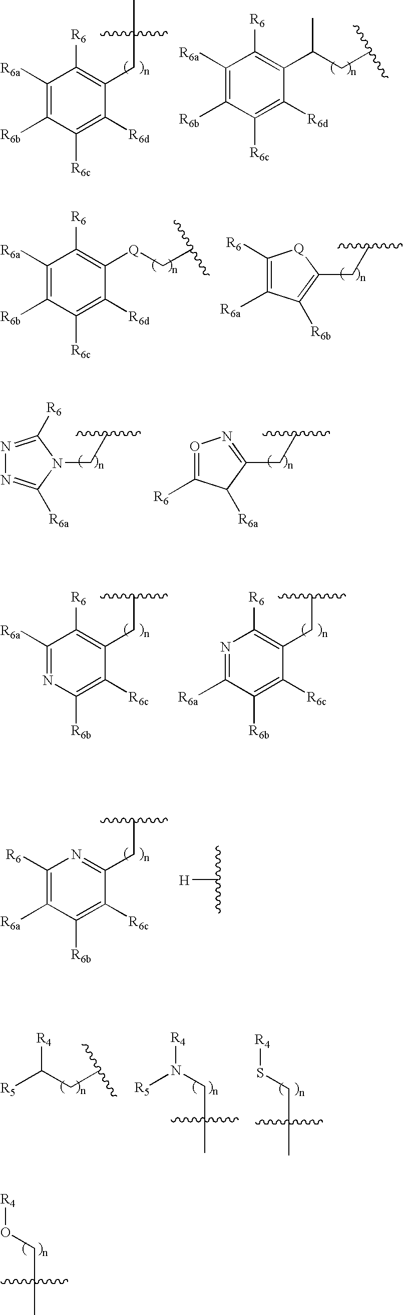
wherein:
n is an integer selected from the group consisting of 0, 1, 2, 3, 4, 5, 6 or 7 defining the number of optionally substituted carbon atoms;
Q is selected from the group consisting of —N(R₄)—, and S;
R₄and R₅ are each independently selected from the group consisting of hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino and protected amino; and
R₆, R₆ₐ, R_{6b}, R_{6c}, and R_{6d} are each independently selected from the group consisting of hydrogen, alkyl, alkenyl, alkynyl, cycloalkyl, cycloalkenyl, cycloalkynyl, aryl, heteroaryl, heteroalicyclyl, aralkyl, heteroaralkyl, (heteroalicyclyl)alkyl, hydroxy, protected hydroxyl, alkoxy, aryloxy, acyl, ester, mercapto, alkylthio, arylthio, cyano, halogen, carbonyl, thiocarbonyl, O-carbamyl, N-carbamyl, O-thiocarbamyt, N-thiocarbamyl, C-amido, N-amido, S-sulfonamido, N-sulfonamido, C-carboxy, protected C-carboxy, O-carboxy, isocyanato, thiocyanato, isothiocyanato, nitro, silyl, sulfenyl, sulfinyl, sulfonyl, haloalkyl, haloalkoxy, trihalomethanesulfonyl, trihalomethanesulfonamido, amino and protected amino; or wherein the substituents selected from the group consisting of R₆, R₆ₐ, R_{6b}, R_{6c}, and R_{6d} can be taken together to form a cycloalkyl, cycloalkenyl, cycloalkynyl, or heteroalicyclyl ring with one or more adjacent members of said group consisting of R₆, R₆ₐ, R_{6b}, R_{6c}, and R_{6d}.

37. (canceled)

38. (canceled)

39. (canceled)

40. (canceled)

41. (canceled)

42. The compound of claim 1, wherein:
X is selected from the group consisting of S, S═O, and SO₂;
Y is —N(R₂) [symbol] or —C(R₁R₂) [symbol] ;
the symbol [symbol] represents a single or double bond, where when [symbol] is a double bond, R₂ is absent;
A is selected from the group consisting of C₃-C₁₂alkyl, C₄-C₁₂alkyl, alkenyl, alkynyl, cycloalkyl, cycloalkenyl, cycloalkynyl, aryl, heteroaryl, aralkyl, and heteroaralkyl, wherein any member of said group can be substituted or unsubstituted;
B, C, D, E, F, C and I are separately selected from the group consisting of hydrogen, alkyl, alkenyl, alkynyl, cycloalkyl, cycloalkenyl, cycloalkynyl, aryl, heteroaryl, aralkyl, heteroaralkyl, heteroalicyclyl, (heteroalicyclyl)alkyl, halogen, hydroxyl, nitro, sulfenyl, sulfinyl, sulfonyl, haloalkyl, haloalkoxy, —CN, —C(=Z)R₁, —C(=Z)OR₁, —C(=Z)NR₁ₐR_{1b}, —C(=Z)N(R₁)NR₁ₐR_{1b}, —C(=Z)N(R₁)N(R₁)C(Z)R₁, —C(R₁)=NR₁, —NR₁ₐ,R_{1b}, —N=CR₁ₐR_{1b}, —N(R₁)—C(=Z)R₁, —N(R₁)—C(=Z)NR₁ₐR_{1b},—S(O)NR₁ₐR_{1b}, —S(O)₂NR₁ₐR_{1b}, —N(R₁)—S(=O)R₁, —N(R₁)—S(═O)₂R₁, —OR₁, —SR₁, and —OC(=Z)R₁, wherein any member of said group can be substituted or unsubstituted except for hydrogen;
H is selected from the group consisting of —C(=Z)NR₁ₐR_{1b}, —C(=Z)N(R₁)NR₁ₐR_{1b}, —C(=Z)N(R₁)N(R₁)C(=Z)R₁, and —C(R₁)=NR₁, wherein any member of said group can be substituted or unsubstituted;
Z is O or S;
R₁, R₁ₐ and R_{1b} are each independently selected from the group consisting of: hydrogen, halogen, alkyl, alkenyl, alkynyl, cycloalkyl, cycloalkenyl, cycloalkynyl, aryl, aralkyl, heteroaryl, heteroaralkyl, heteroalicyclyl, (heteroalicyclyl)alkyl, —(CH₂)₀₋₇—OR₃, —(CH₂)₀₋₇—SR₃, —(CH₂)₀₋₇—NR₃ₐR_{3b}, haloalkyl, —C(=Z)R₃, —C(=Z)OR₃ₐR_{3b}, wherein any member of said group can be substituted or unsubstituted except for hydrogen;
or R₁ₐ and R_{1b} can be taken together to form an unsubstituted or substituted heteroalicyclyl having 2 to 9 carbon atoms or an unsubstituted or substituted carbocyclyl having 3 to 9 carbon atoms;
R₂ is absent or is selected from the group consisting of: hydrogen, alkyl, alkenyl, alkynyl, cycloalkyl, cycloalkenyl, cycloalkynyl, aryl, heteroaryl, and heteroalicyclyl, wherein any member of said group can be substituted or unsubstituted except for hydrogen; and
R₃, R₃ₐ, and R_{3b} are each independently selected from the group consisting of: hydrogen, alkyl, alkenyl, alkynyl, cycloalkyl, cycloalkenyl, aryl, heteroaryl, heteroalicyclyl, aralkyl, heteroaralkyl, and (heteroalicyclyl)alkyl, wherein any member of said group can be substituted or unsubstituted except for hydrogen.

43. The compound of claim 42, wherein Z is O.

44. (canceled)

45. (canceled)

46. (canceled)

47. The compound of claim 42, wherein:
A is selected from the group consisting of C₃-C₁₂alkyl, C₄-C₁₂alkyl, cycloalkyl, aryl, heteroaryl, heteroalicyclyl, halogen, —NR₁ₐR_{1b}, and —(CH₂)₀₋₄—C(=Z)—OR₁;
X is S;
Y is —N(R₂) [symbol] wherein the symbol represents a double bond and R₂ does not exist; and
H is —C(=Z)NR₁ₐR_{1b}.

48. (canceled)

49. (canceled)

50. (canceled)

51. (canceled)

52. (canceled)

53. (canceled)

54. (canceled)

55. (canceled)

56. The compound of claim 55, wherein R₁ₐ is selected from the group consisting of: 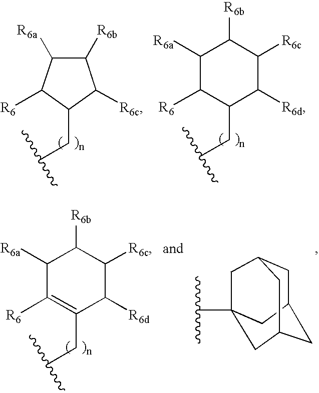
wherein n is 1 or 2.

57. (canceled)

58. (canceled)

59. The compound of claim 47, wherein R₁ₐ is selected from the group consisting of: 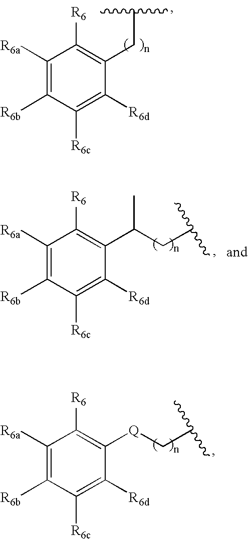
wherein Q is —N(R₄)—, oxygen or sulfur; and R₄ is hydrogen or C₁₋₄alkyl, wherein n is 1 or 2.

60. (canceled)

61. (canceled)

62. The compound of claim 47, wherein the R₁ₐ is selected from the group consisting of: 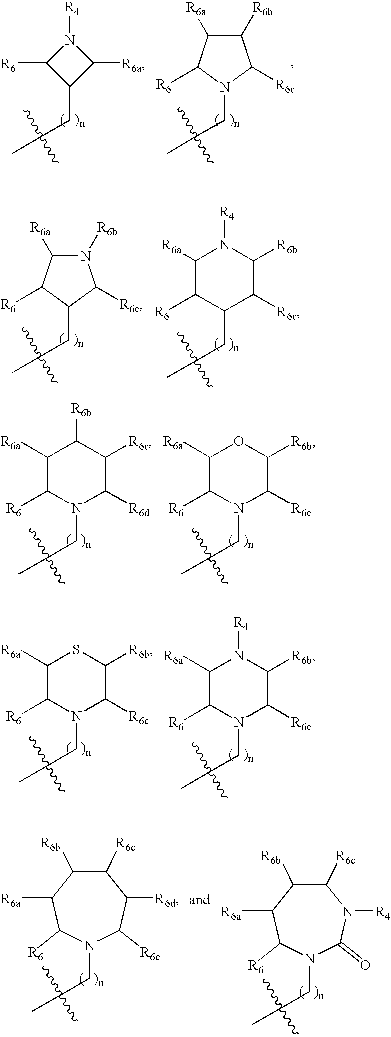
wherein n is 1 or 2.

63. (canceled)

64. (canceled)

65. The compound of claim 47, wherein e R₁ₐ is from the group consisting of: 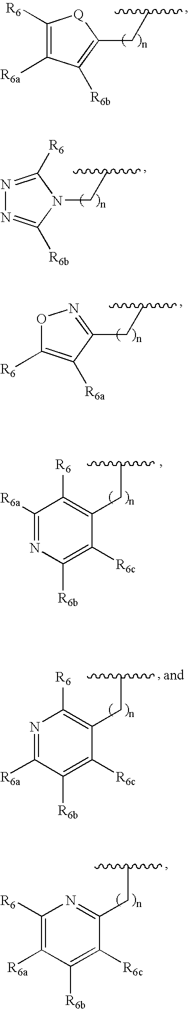
wherein Q is oxygen or sulfur wherein n is 1 or 2.

66. (canceled)

67. (canceled)

68. The compound of claim 1, wherein the compound is selected from the group consisting of:

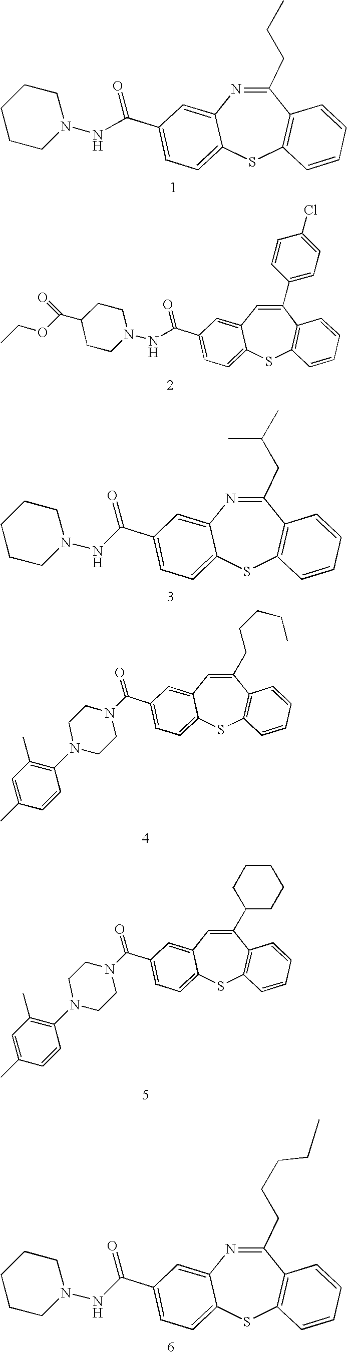

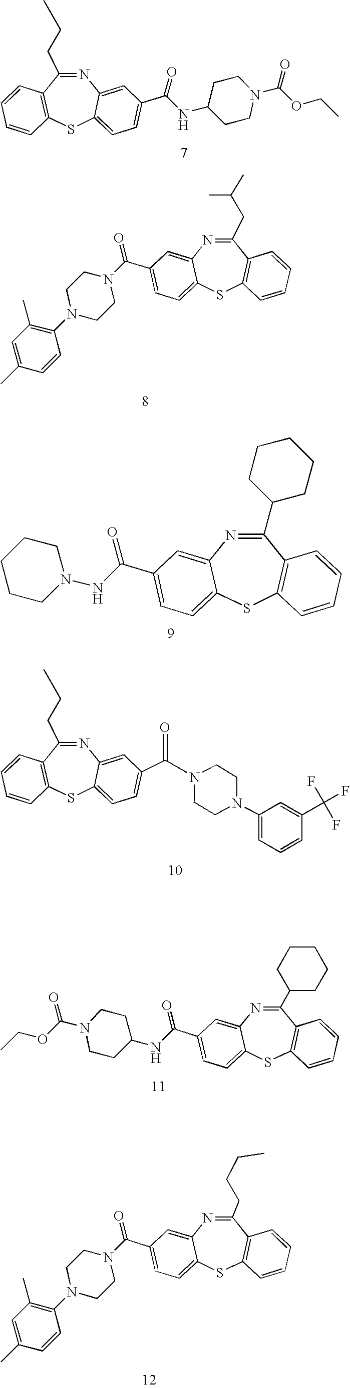

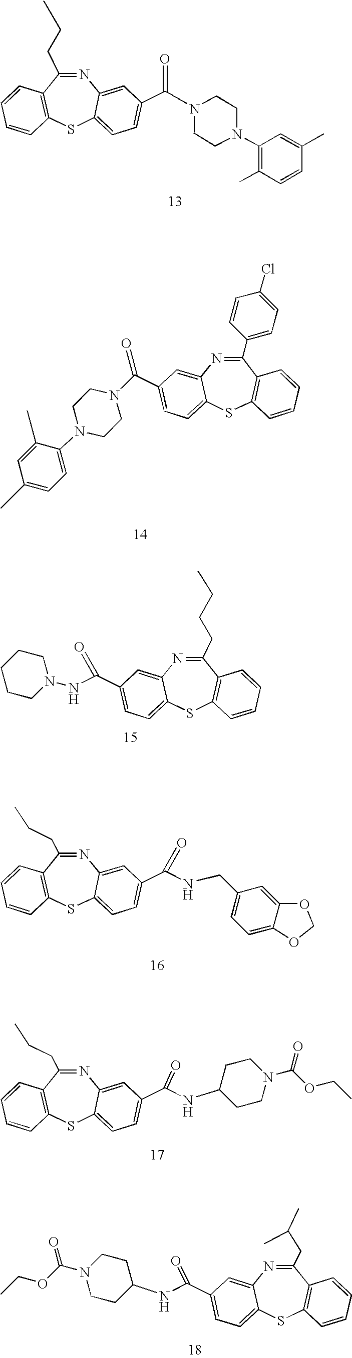

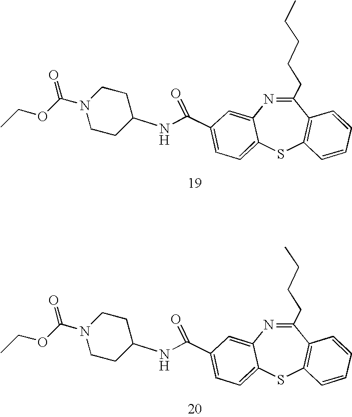

69. The compound of claim 1, wherein the compound is selected from the group consisting of:

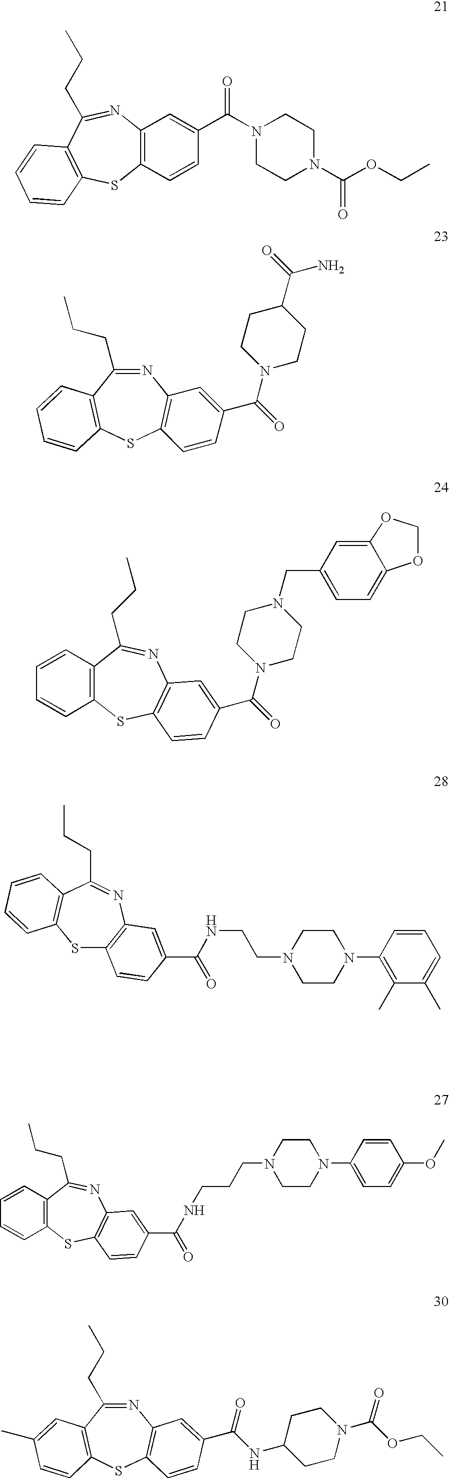

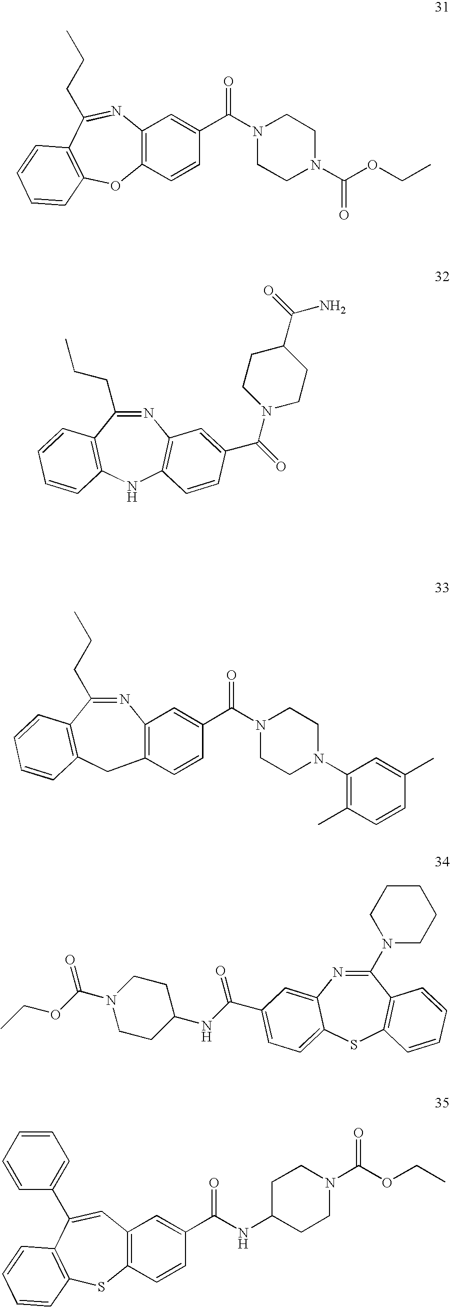

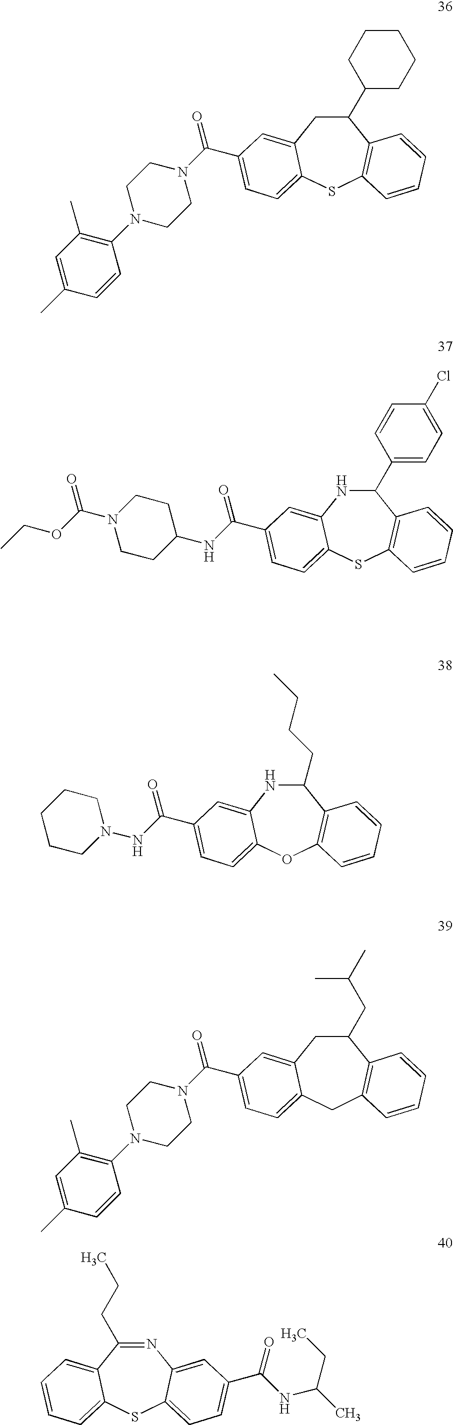

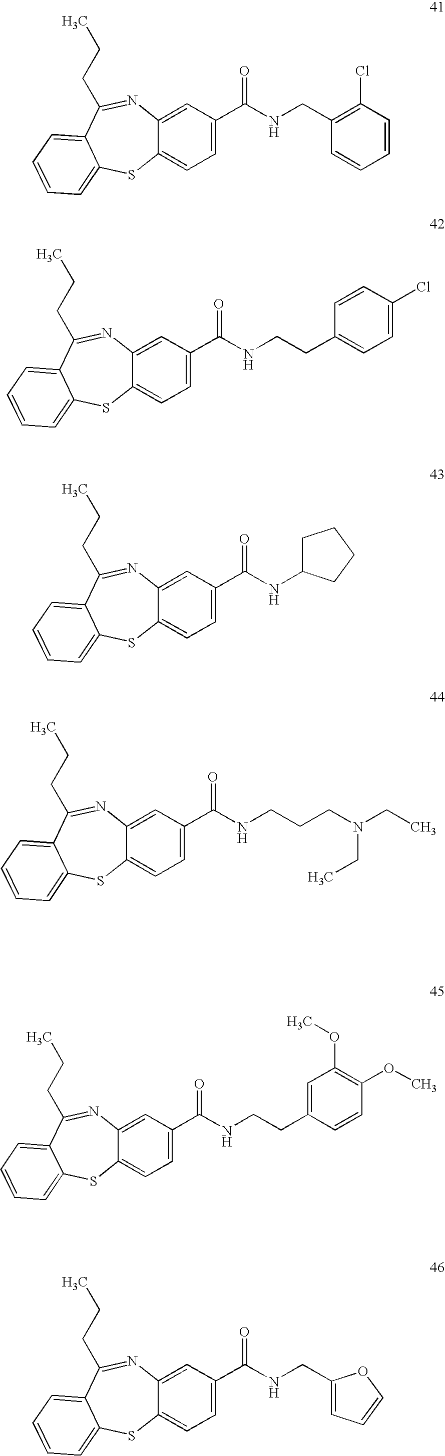

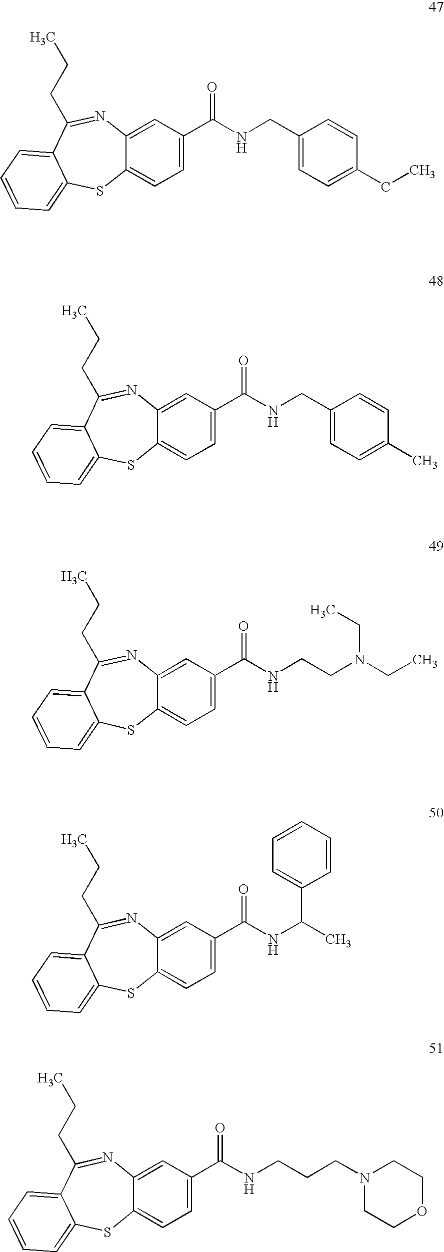

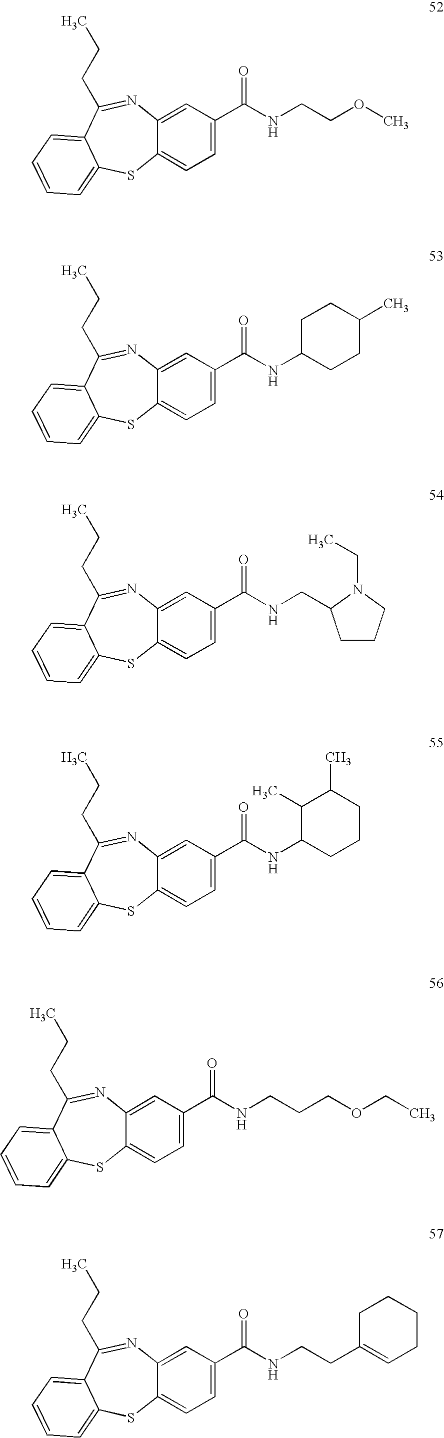

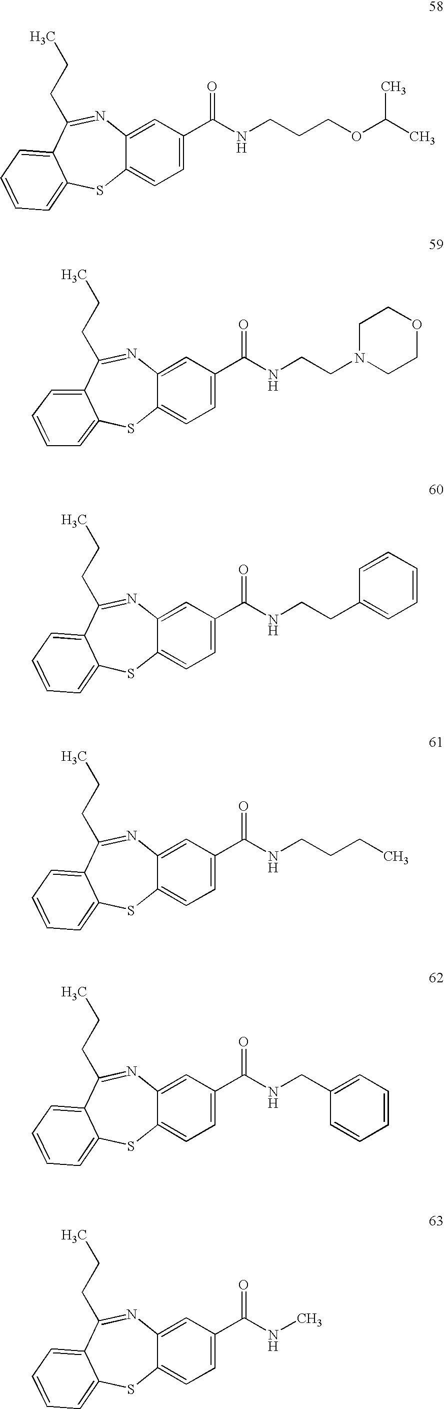

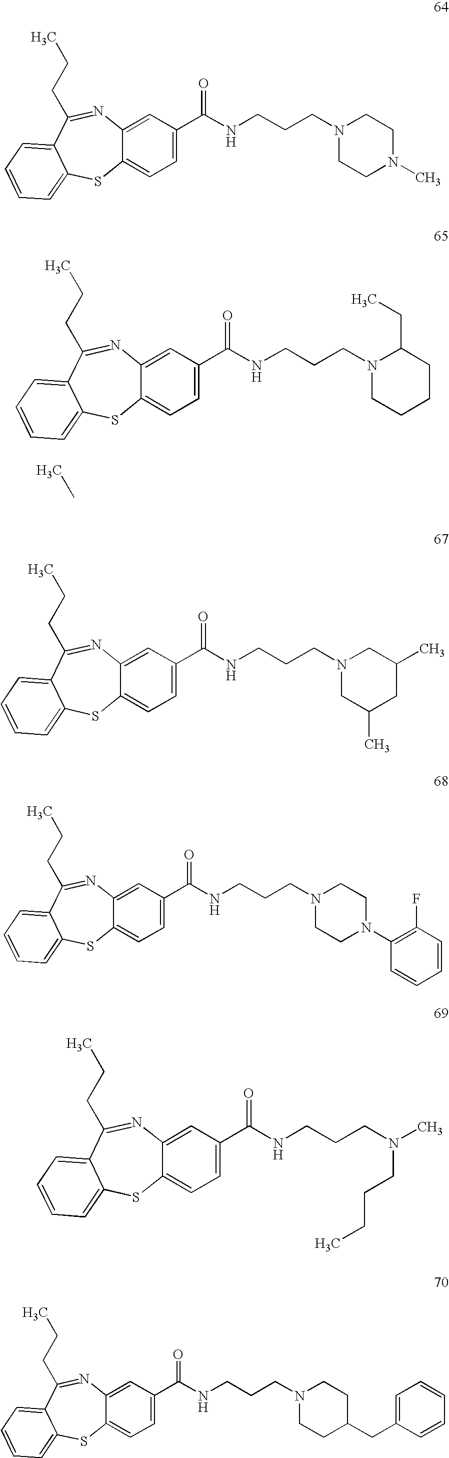

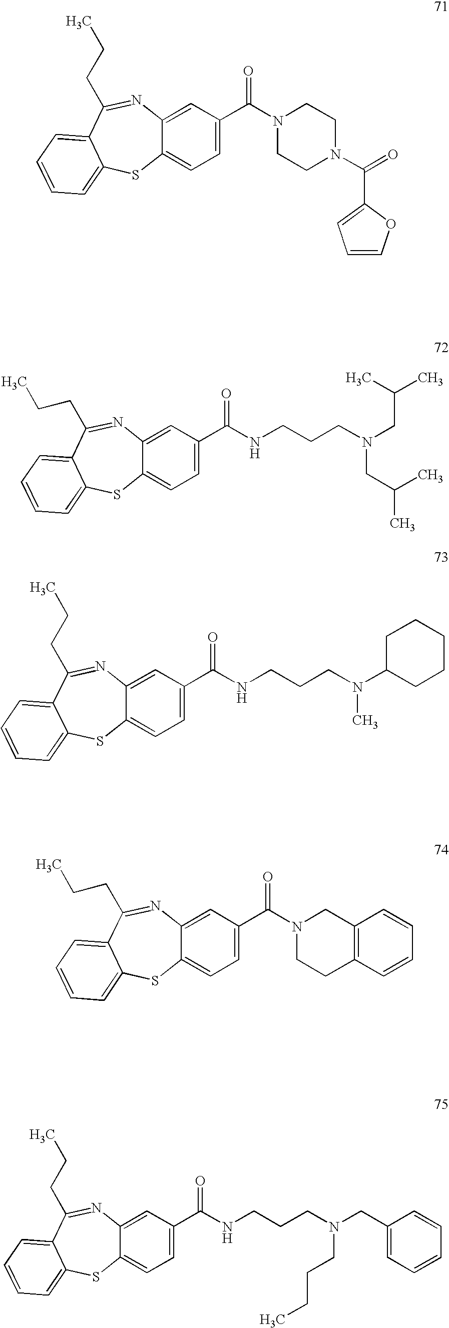

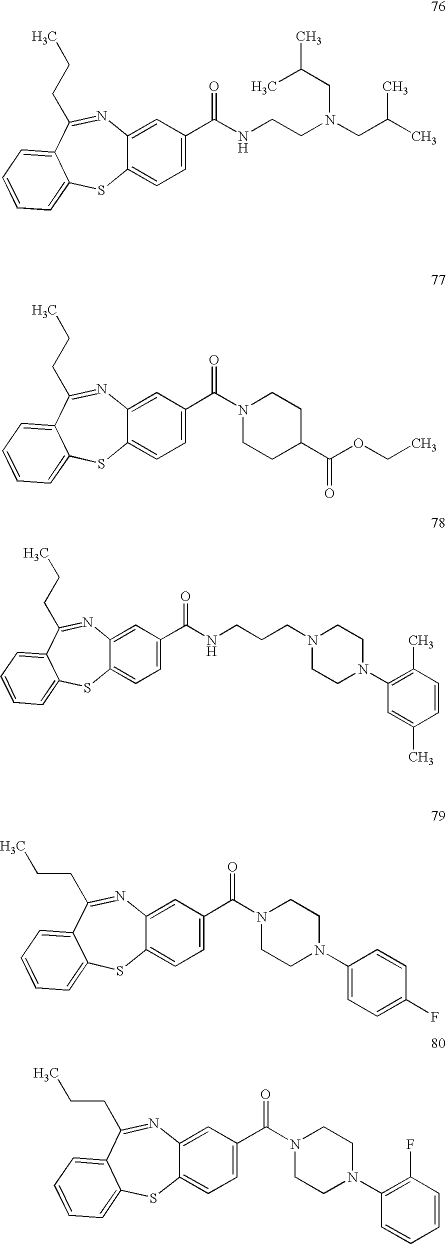

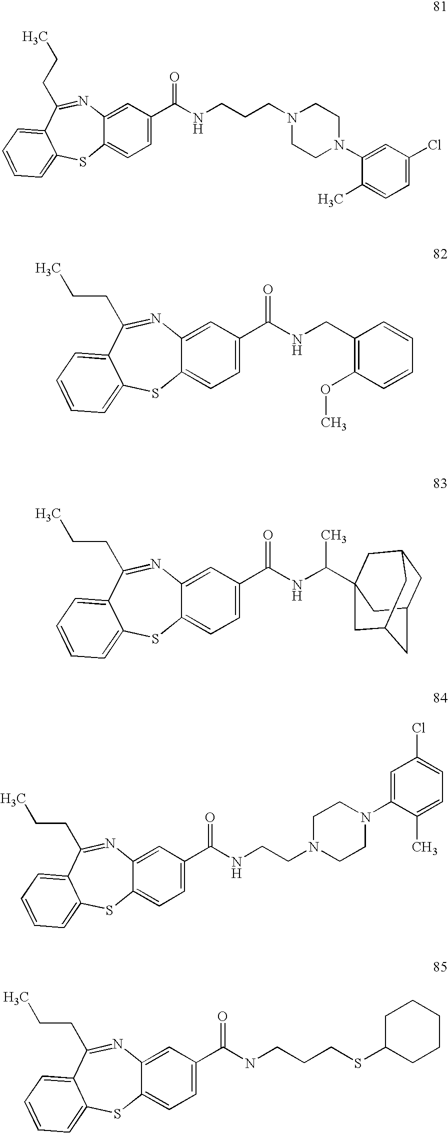

70. The compound of claim 1, wherein the compound is selected from the group consisting of:

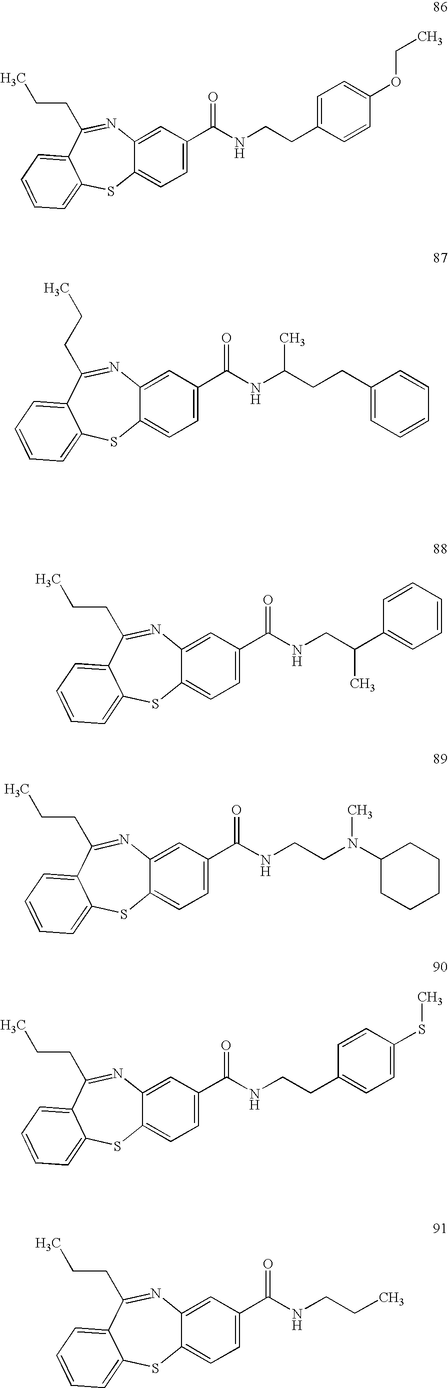

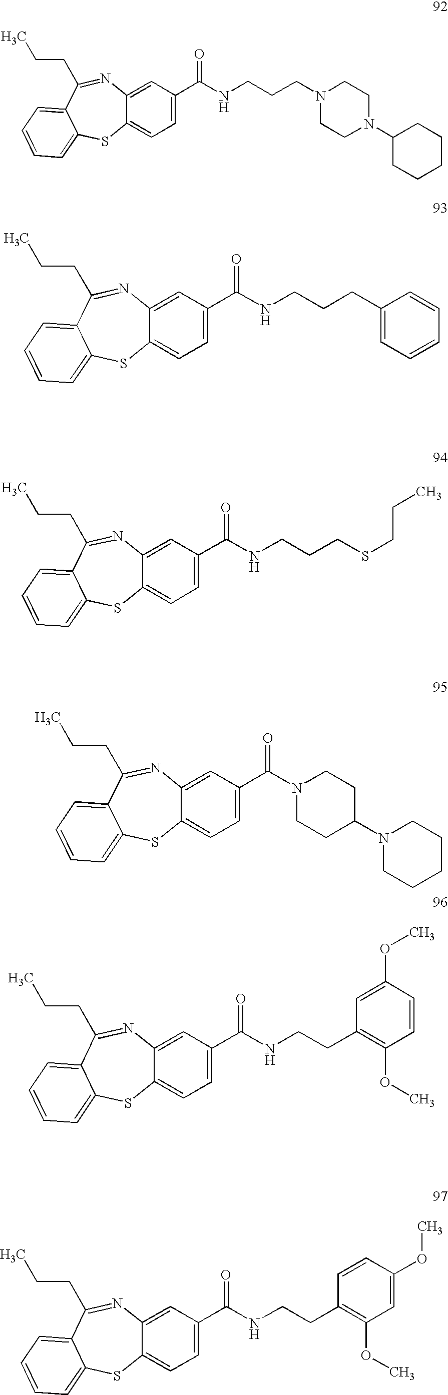

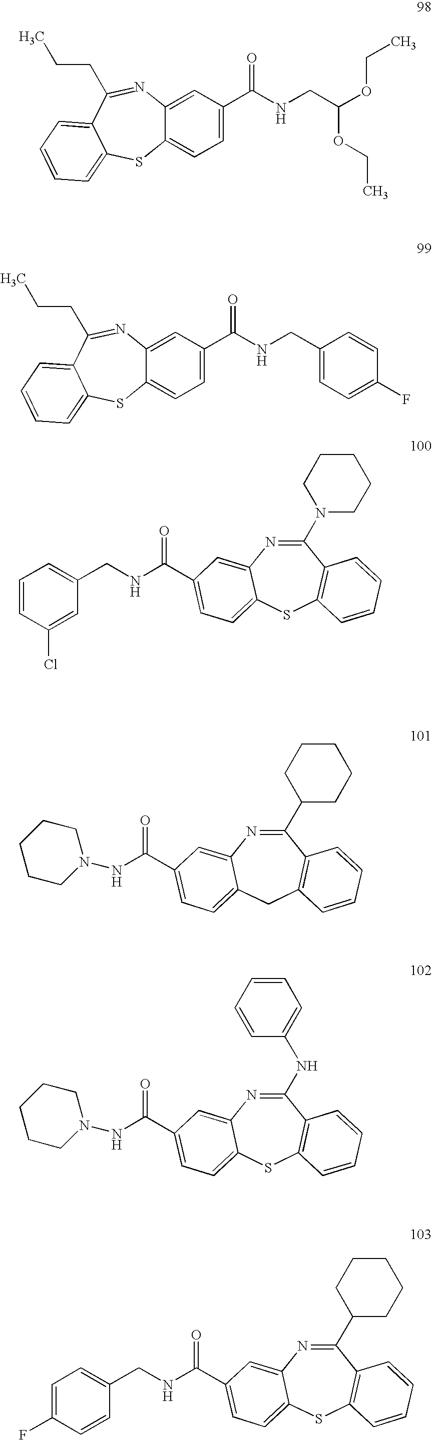

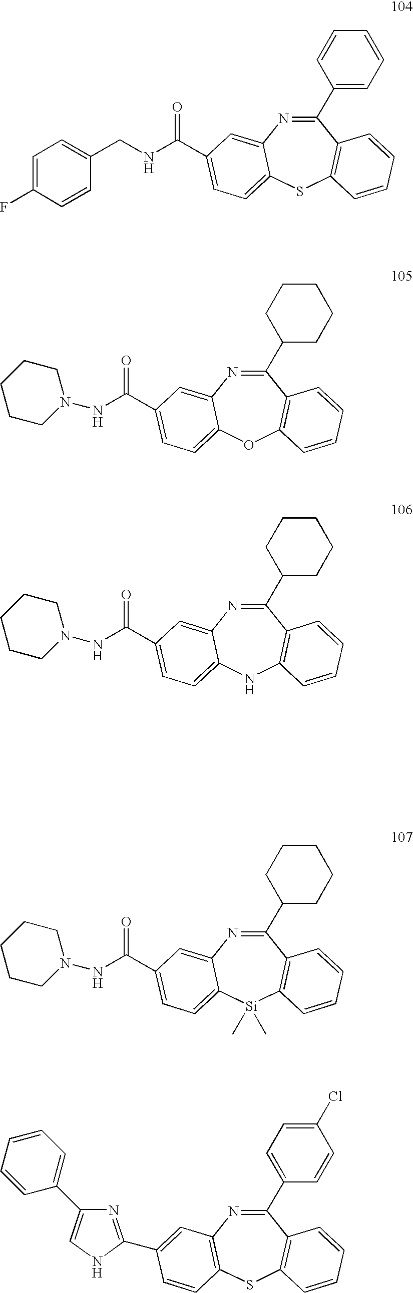

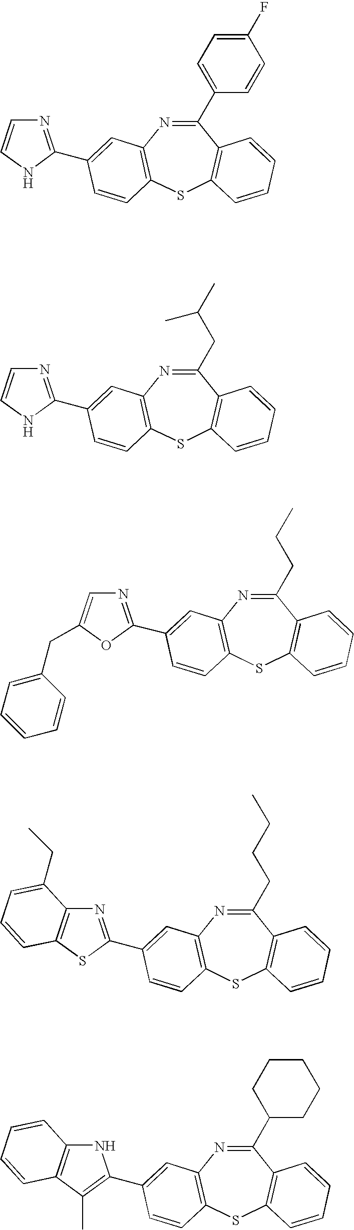

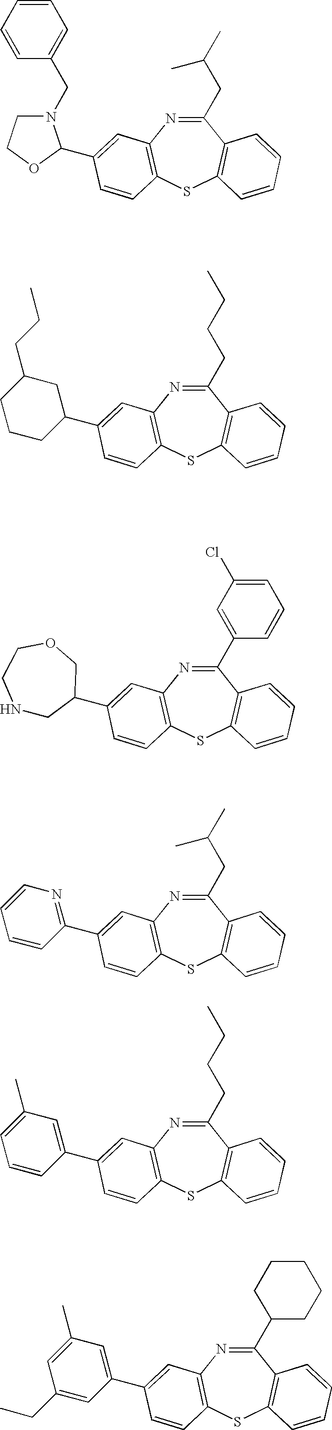

71. The compound of claim 1, wherein the compound is selected from the group consisting of:

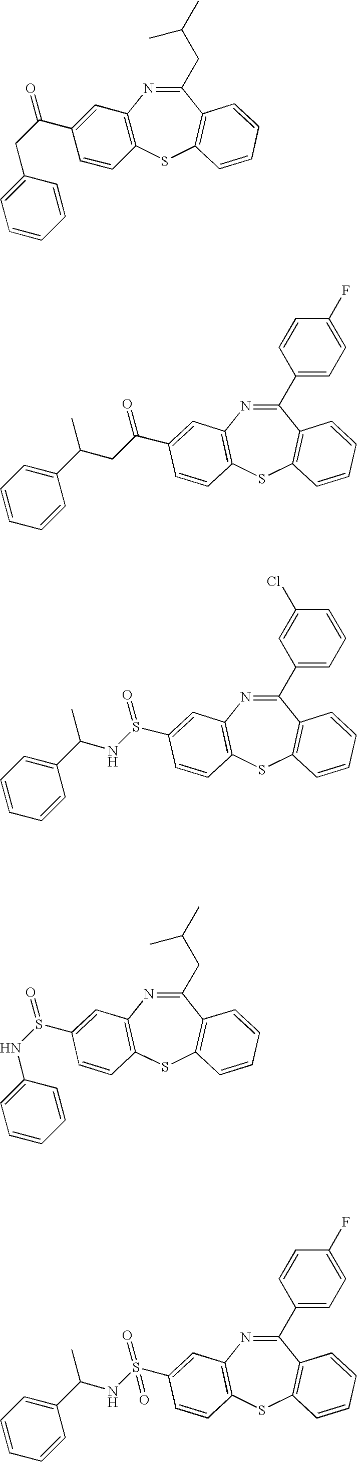

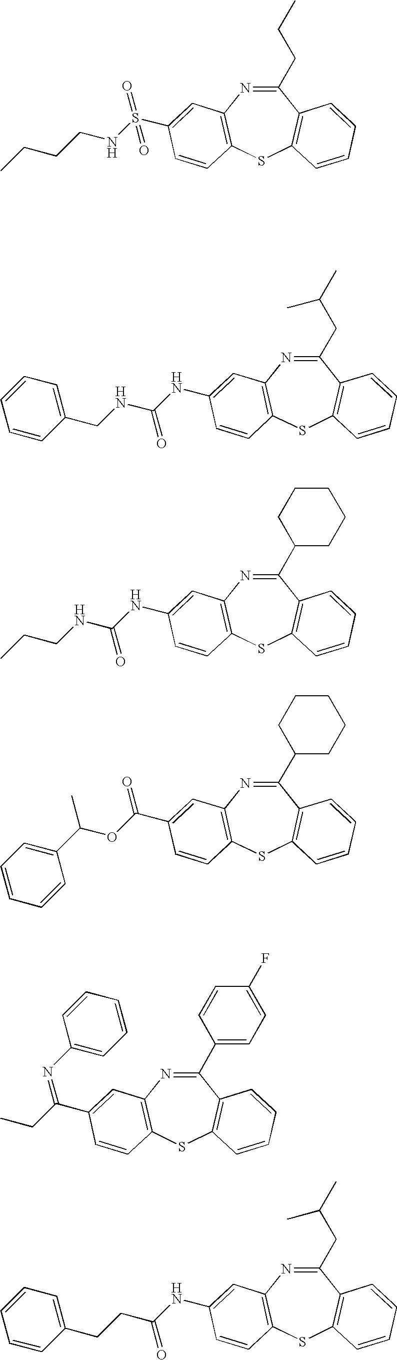

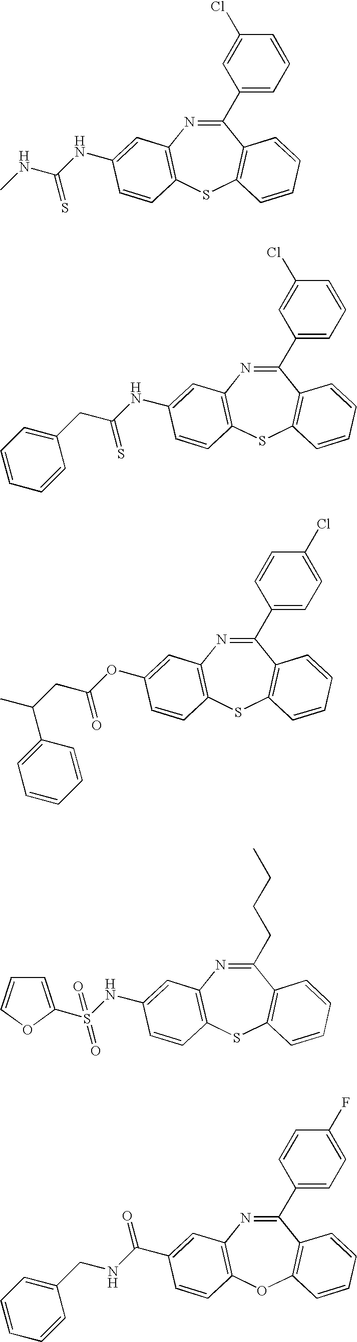

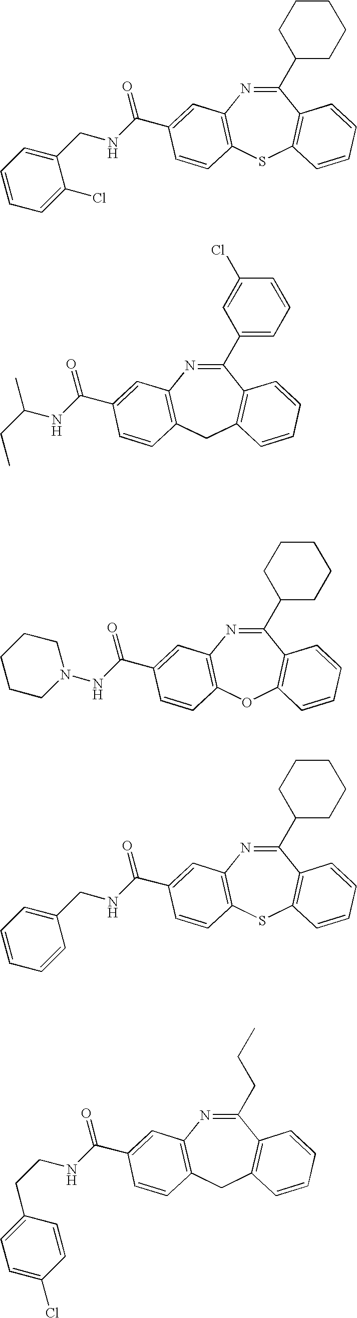

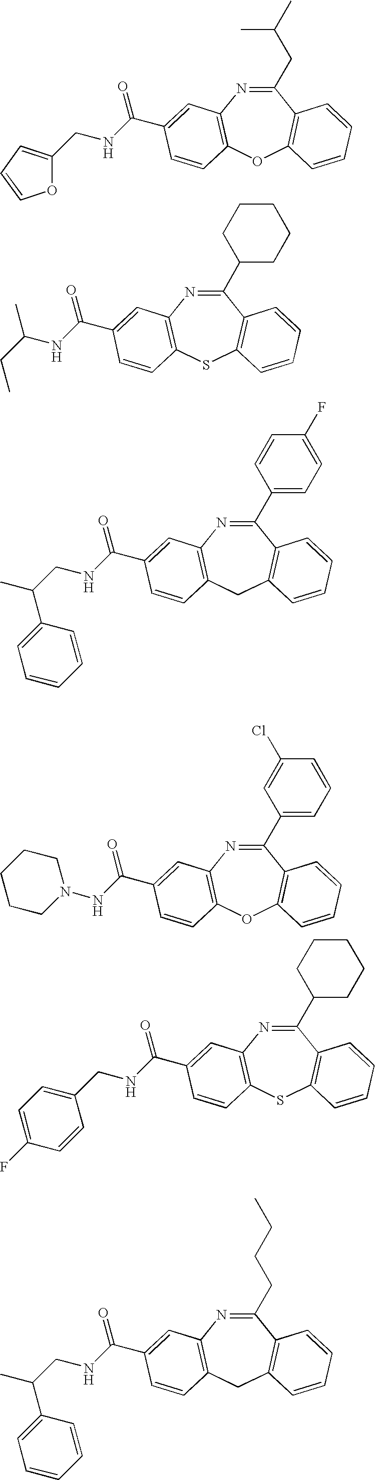

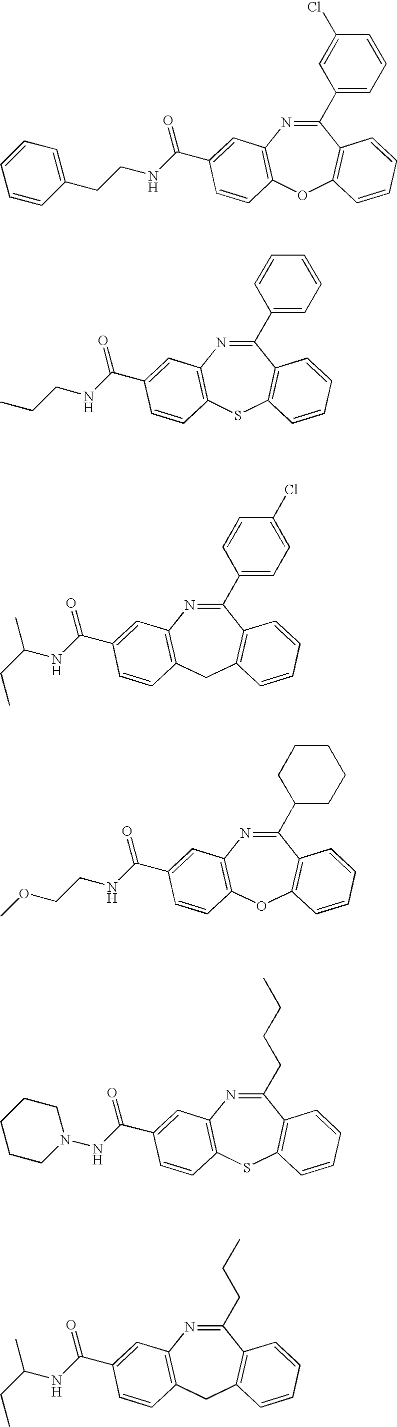

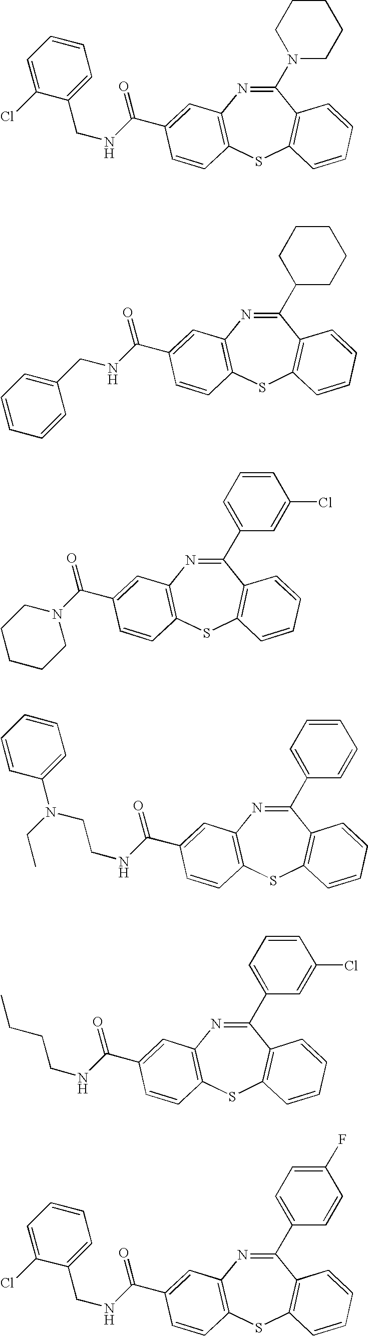

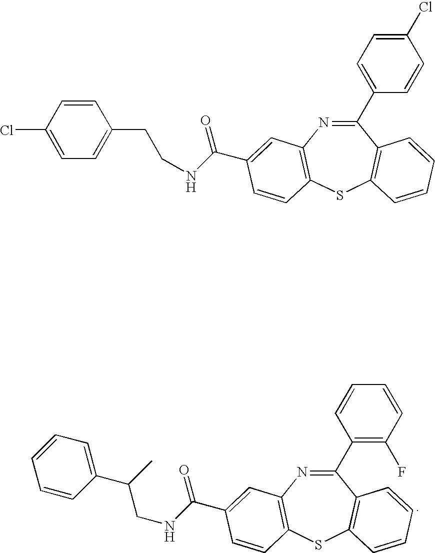

72. The compound of claim 1, wherein the compound is selected from the group consisting of:

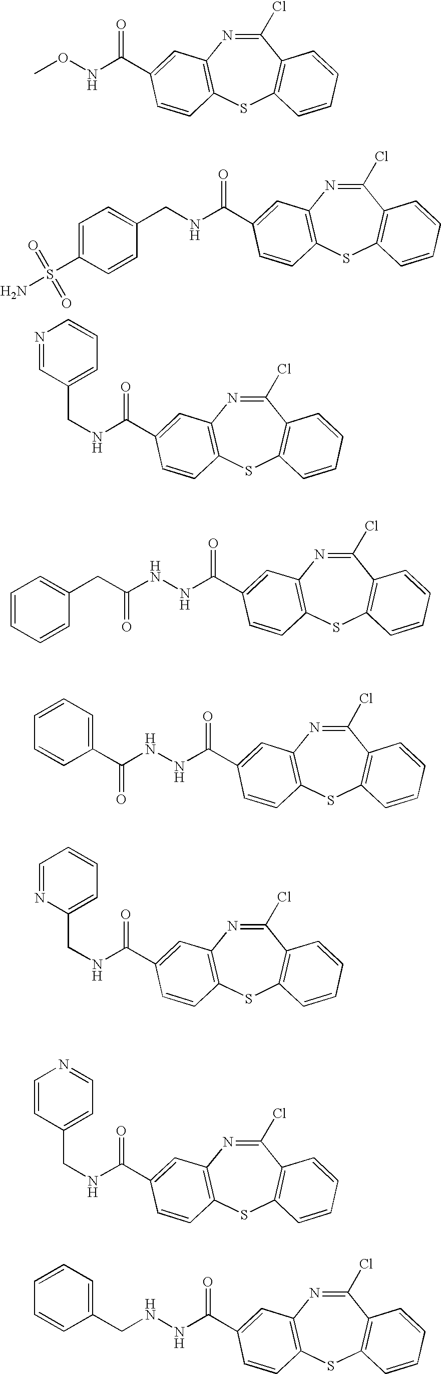

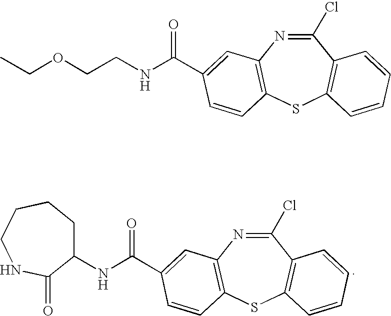

73. The compound of claim 1, wherein the compound is selected from the group consisting of:

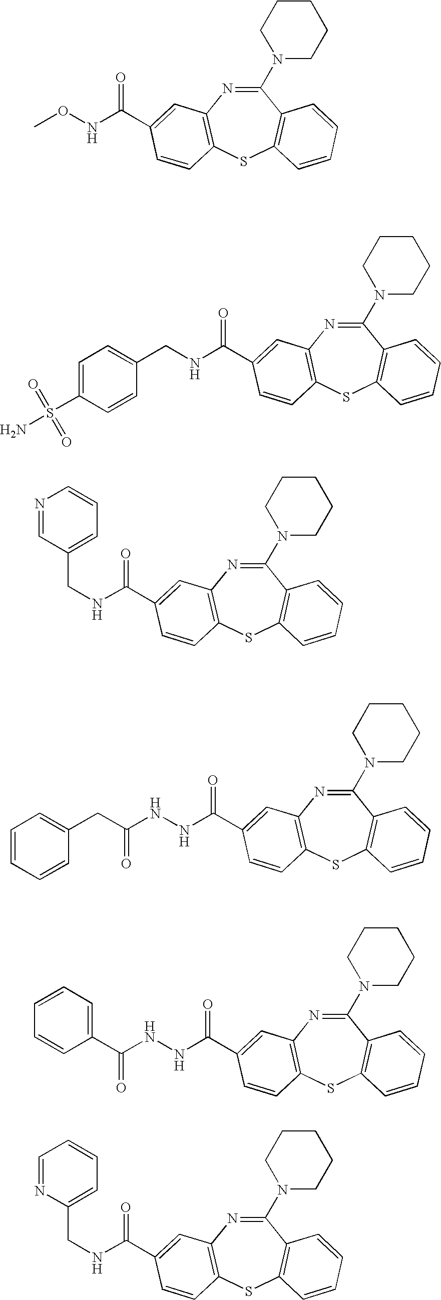

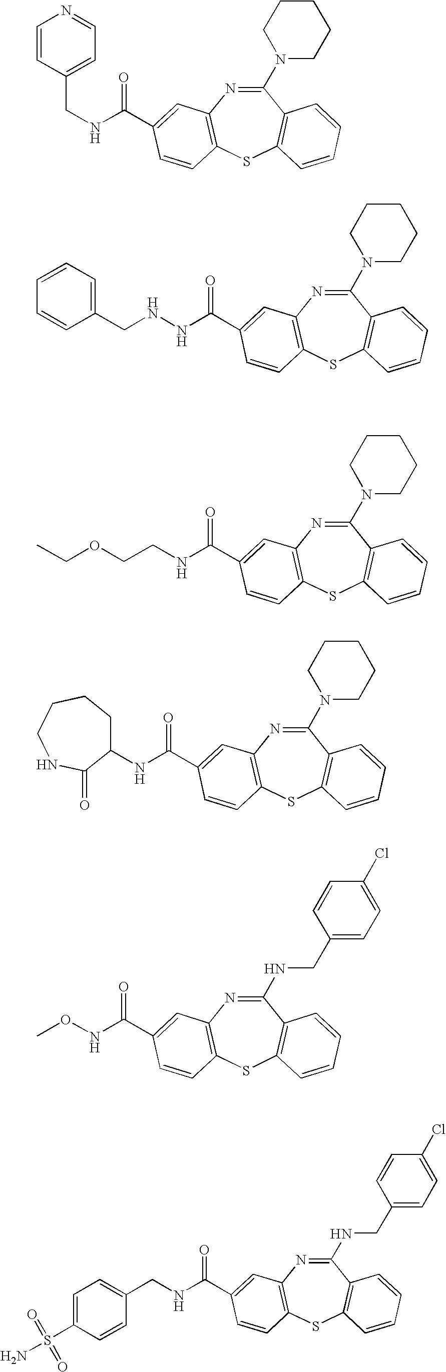

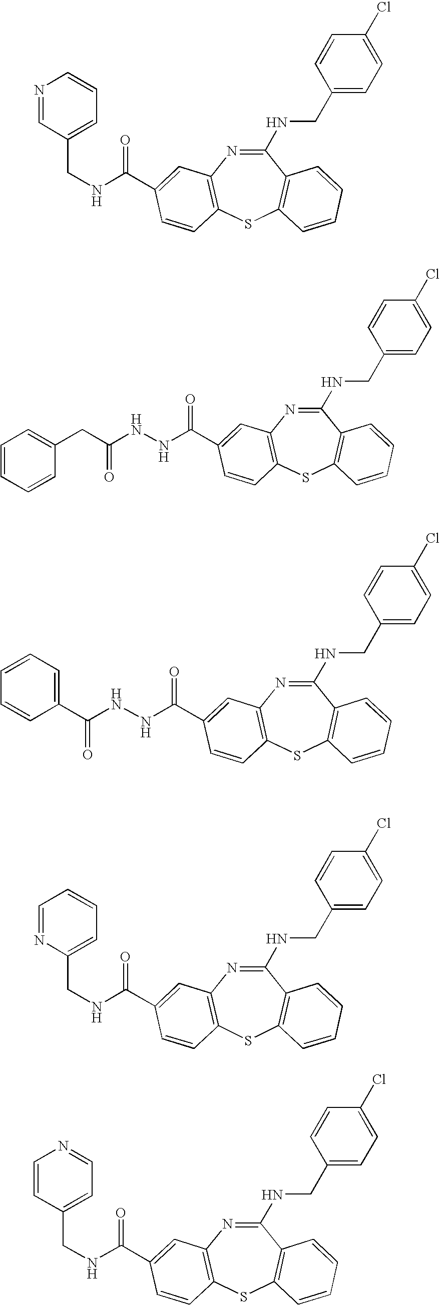

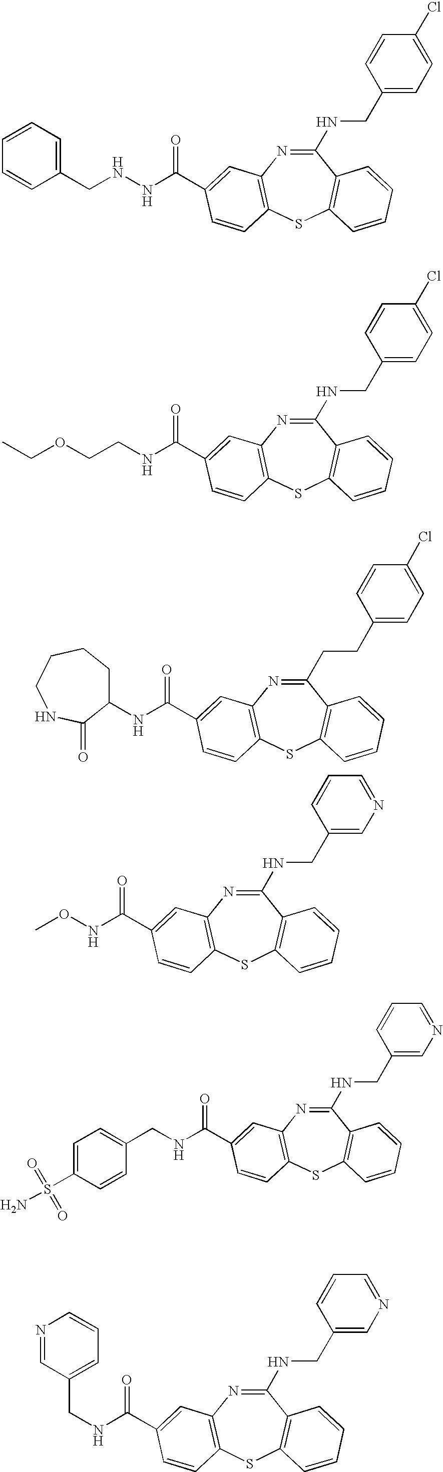

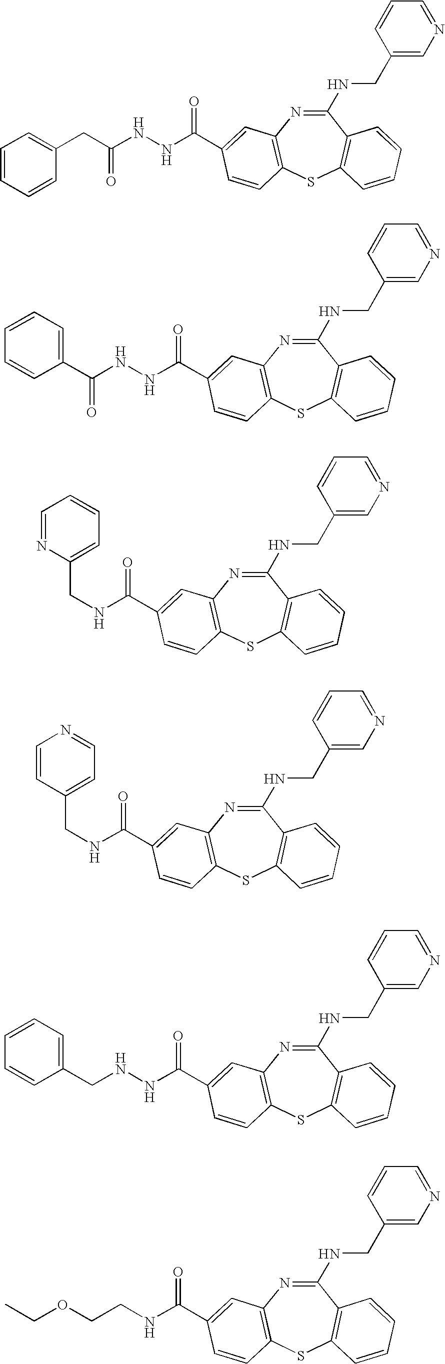

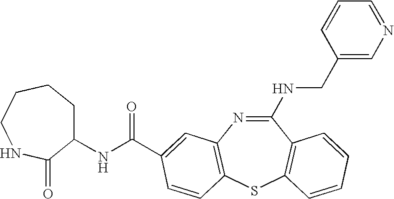

74. The compound of claim 1, wherein the compound is selected from the group consisting of:

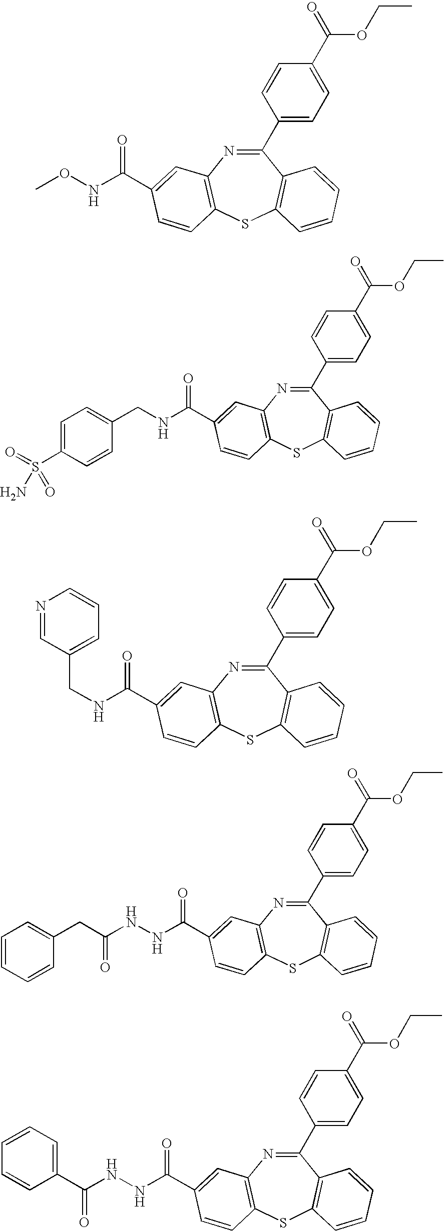

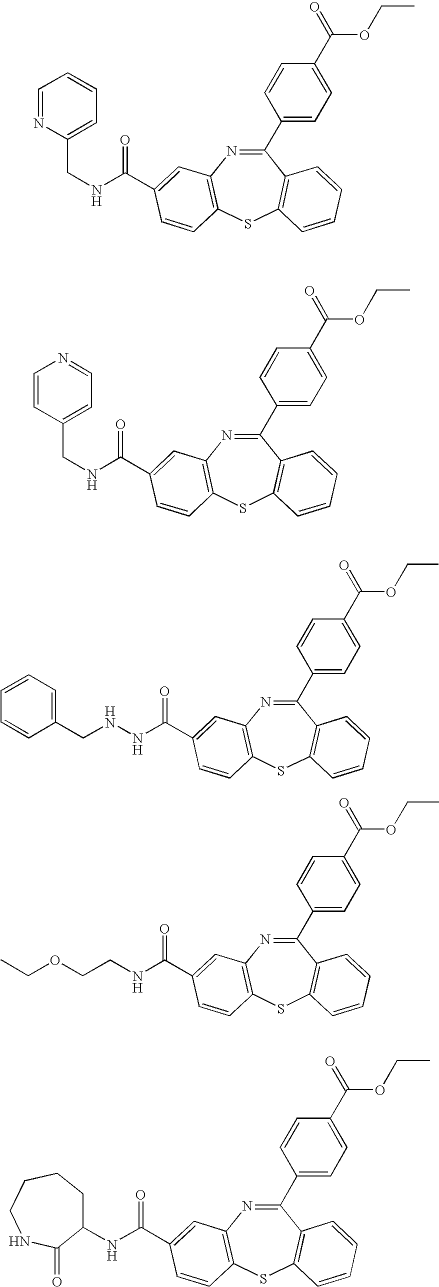

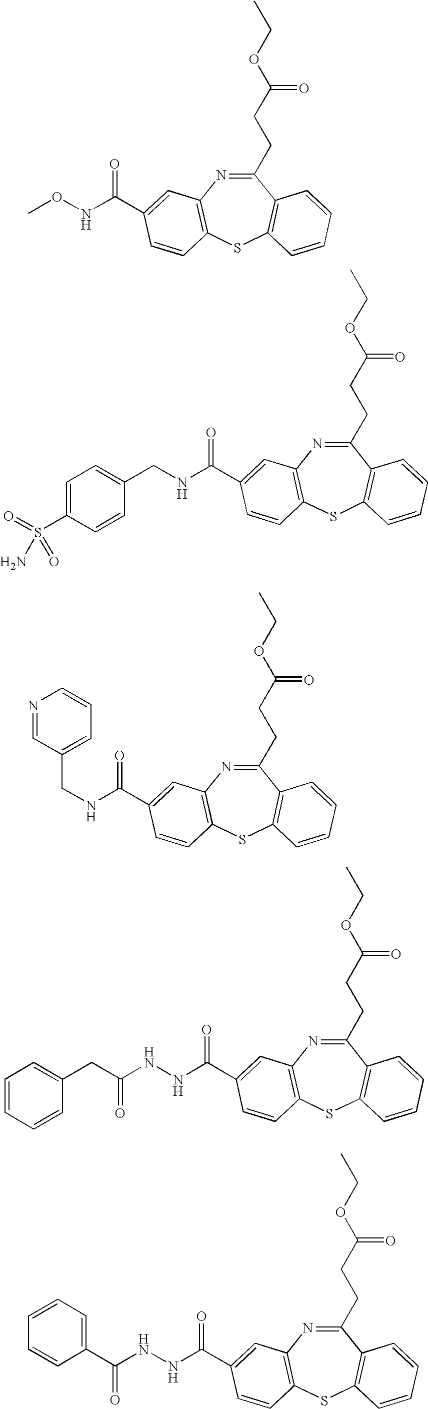

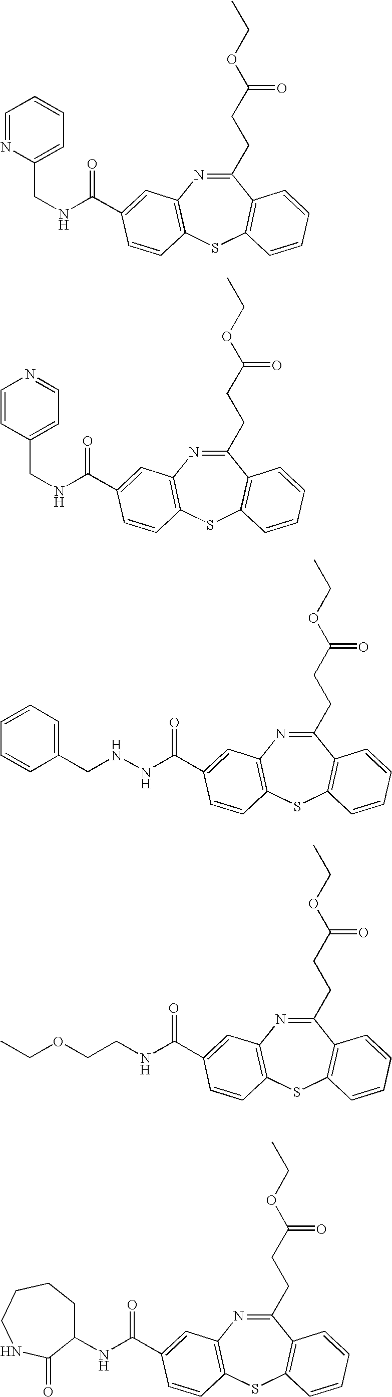

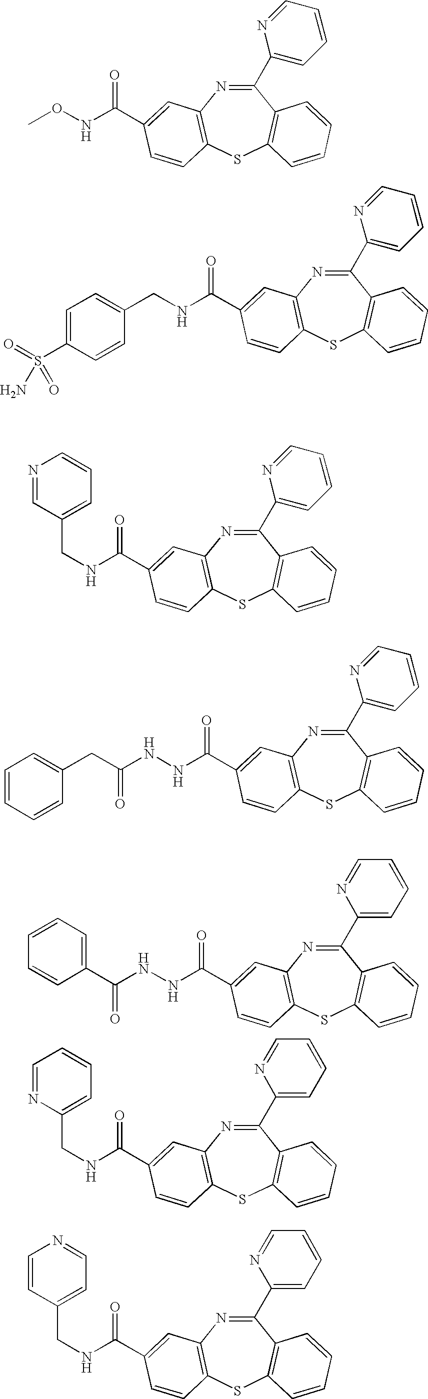

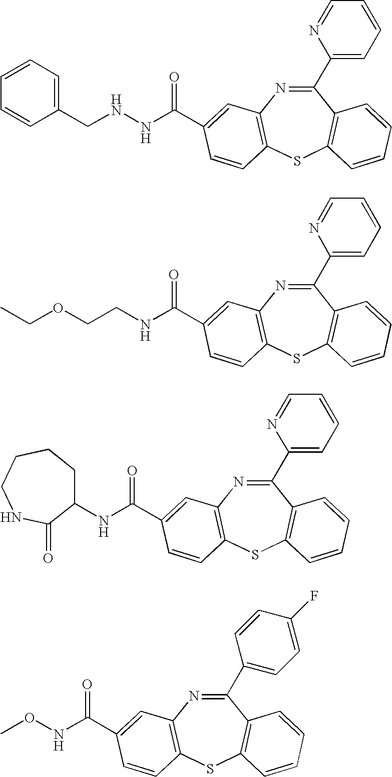

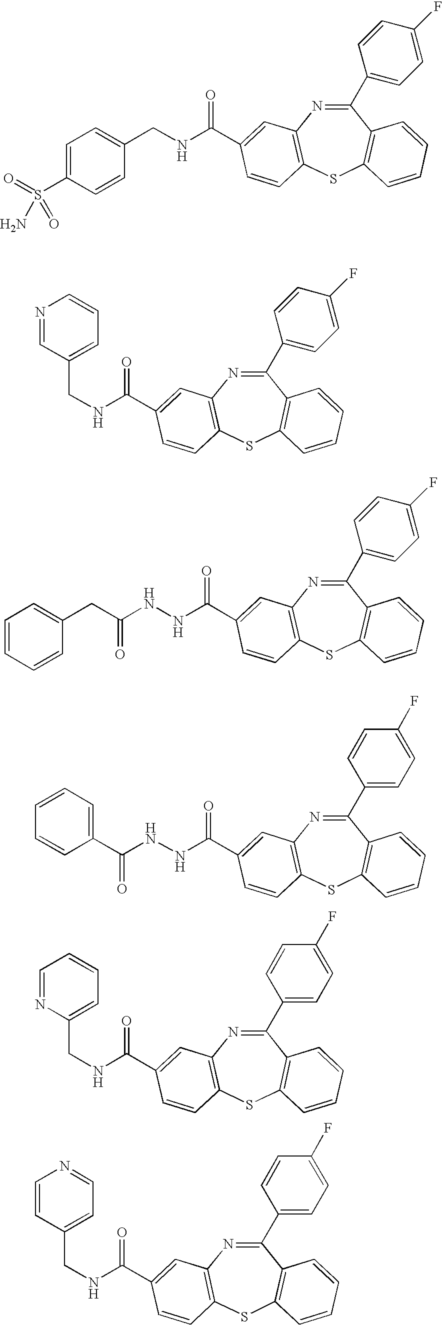

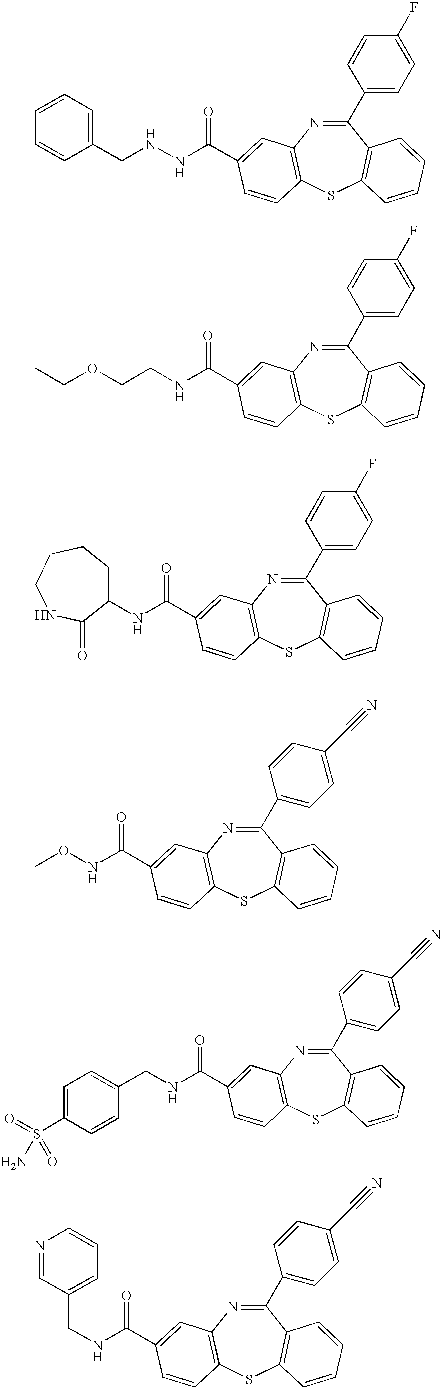

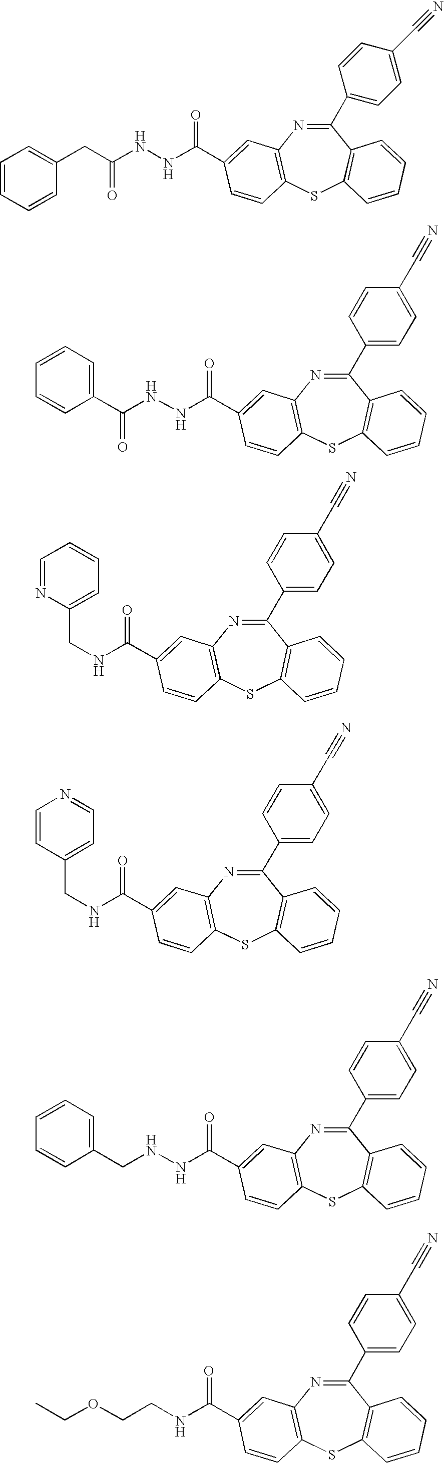

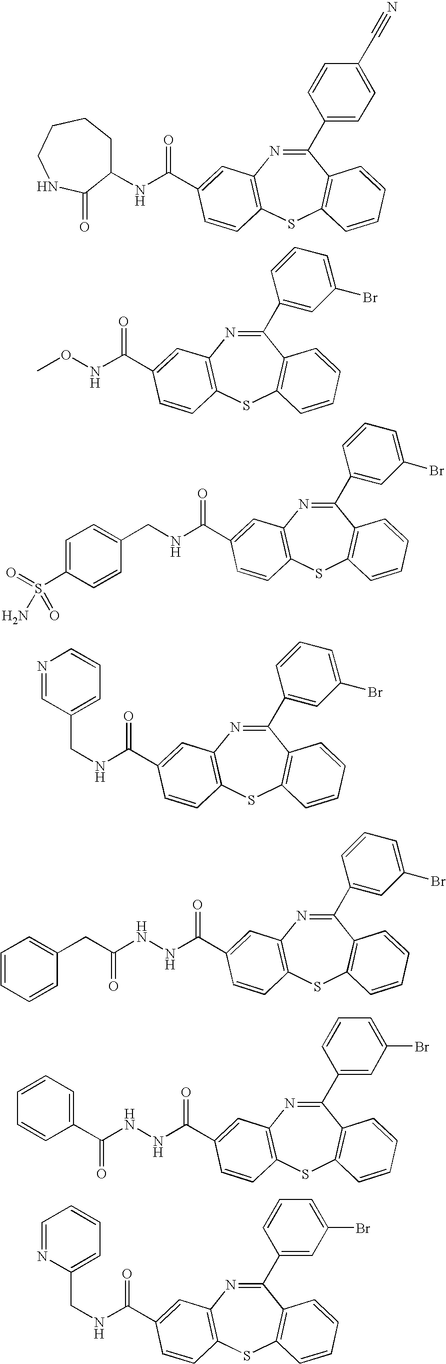

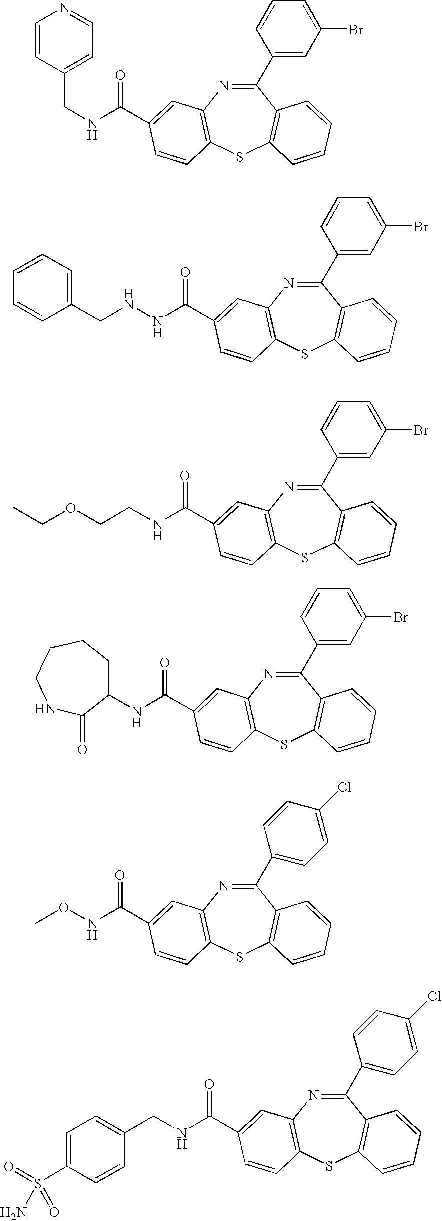

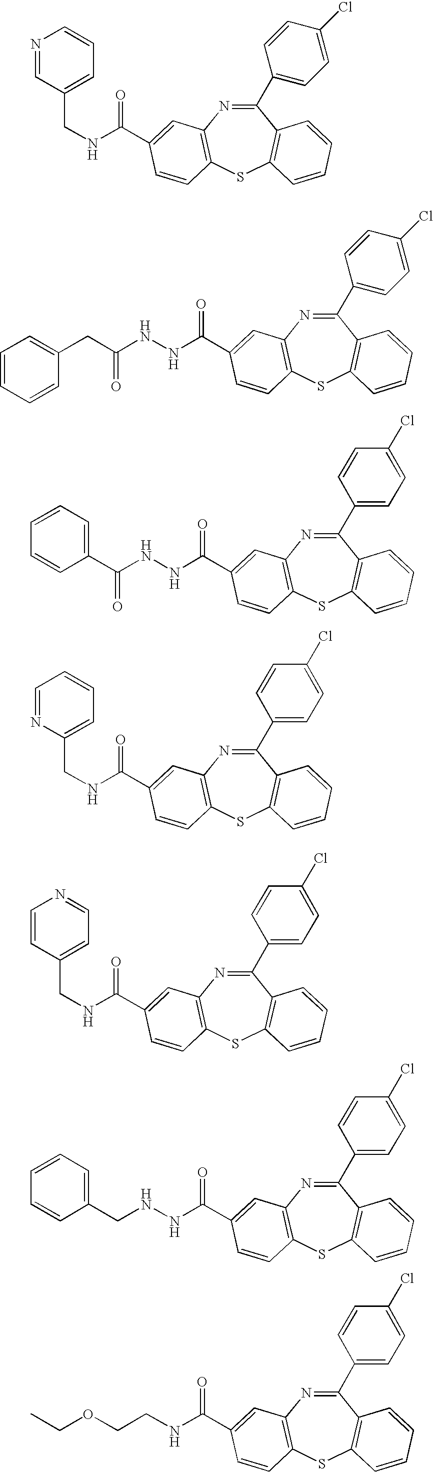

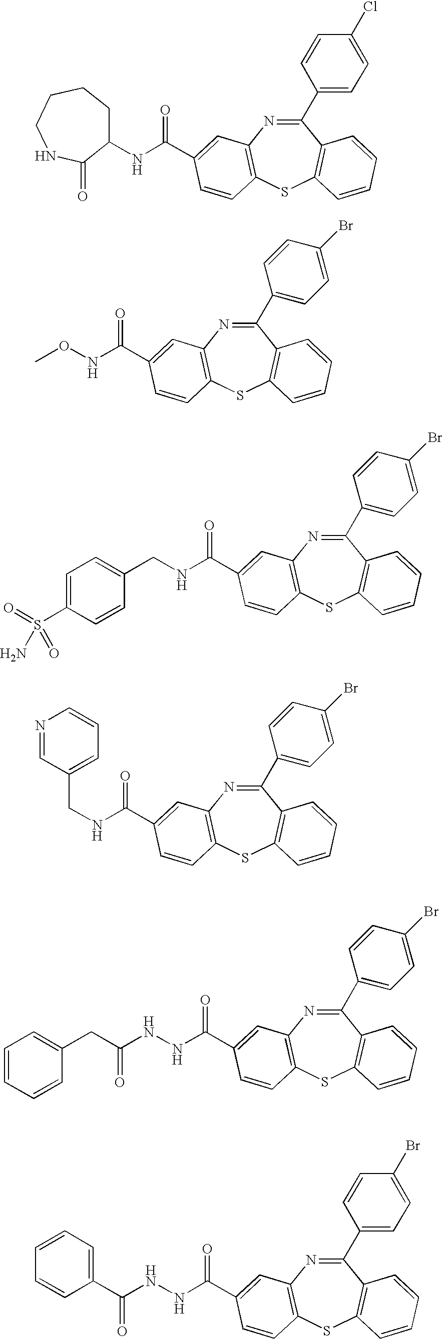

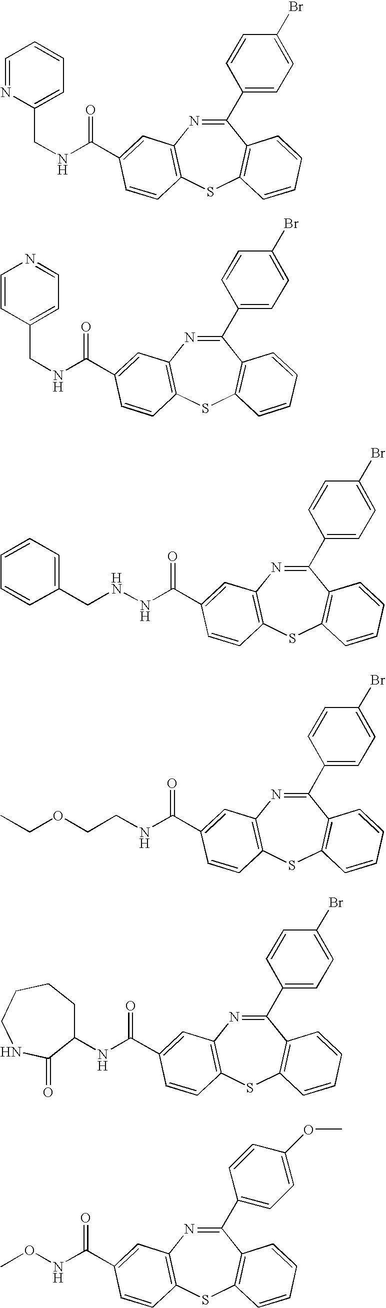

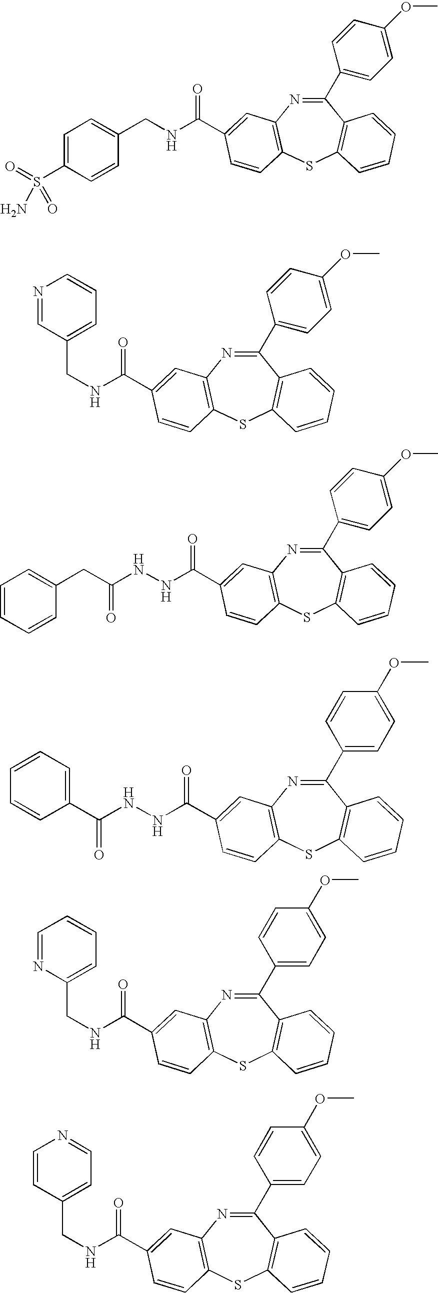

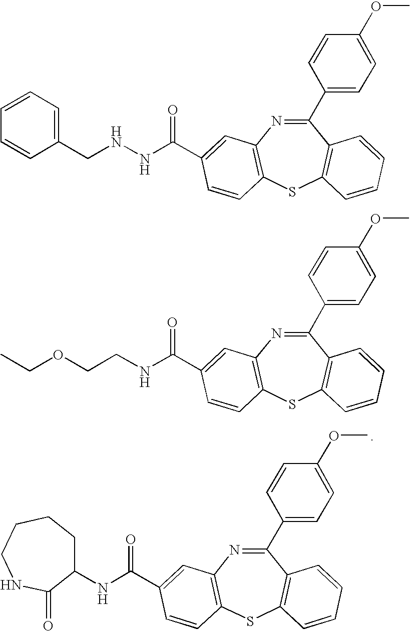

75. The compound of claim 1, wherein the compound is selected from the group consisting of:

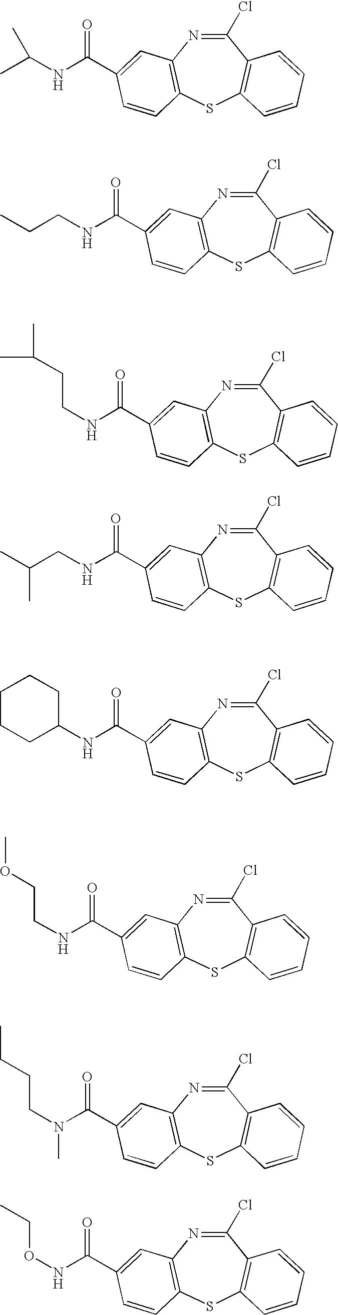

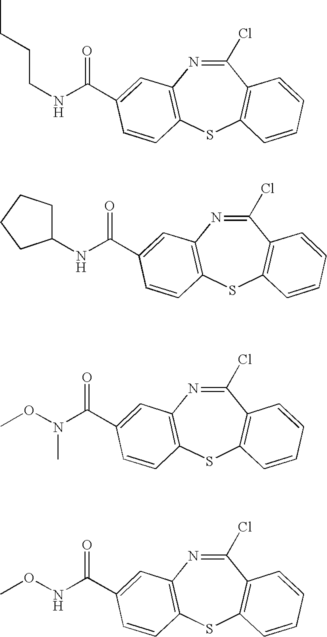

76. The compound of claim 1, wherein the compound is selected from the group consisting of:

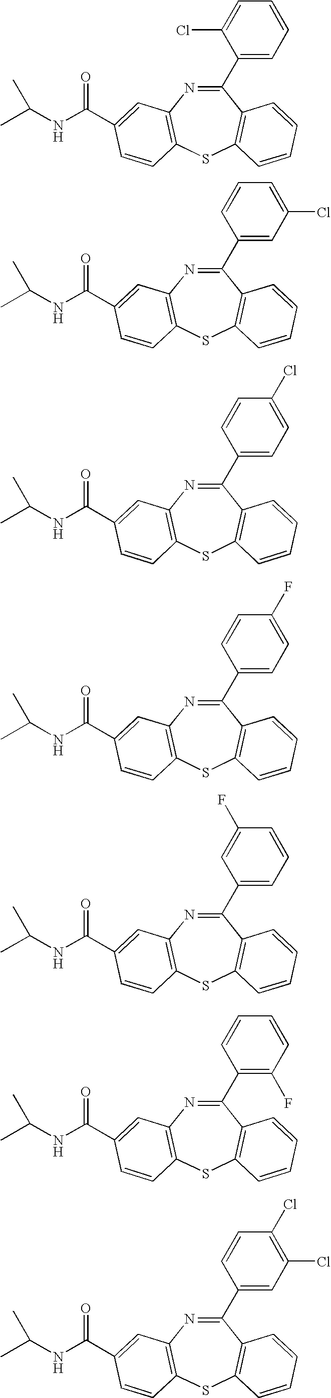

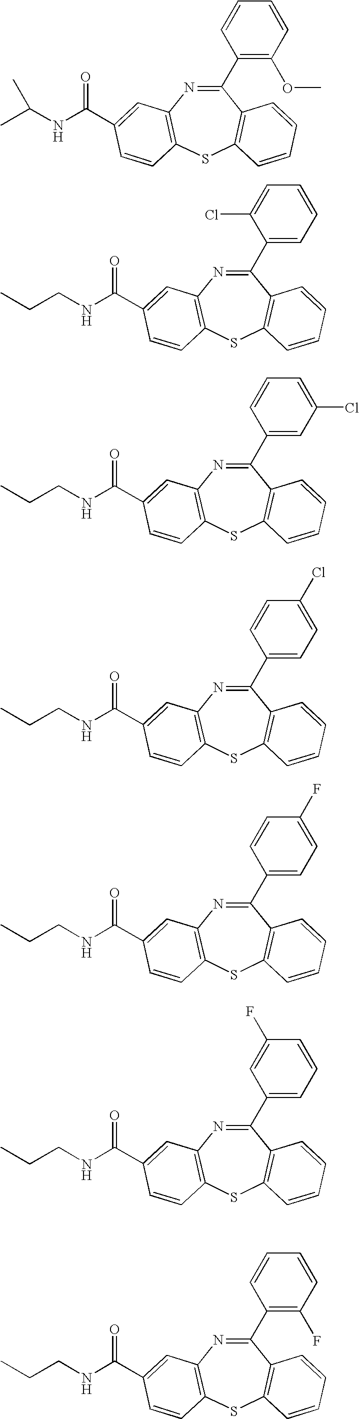

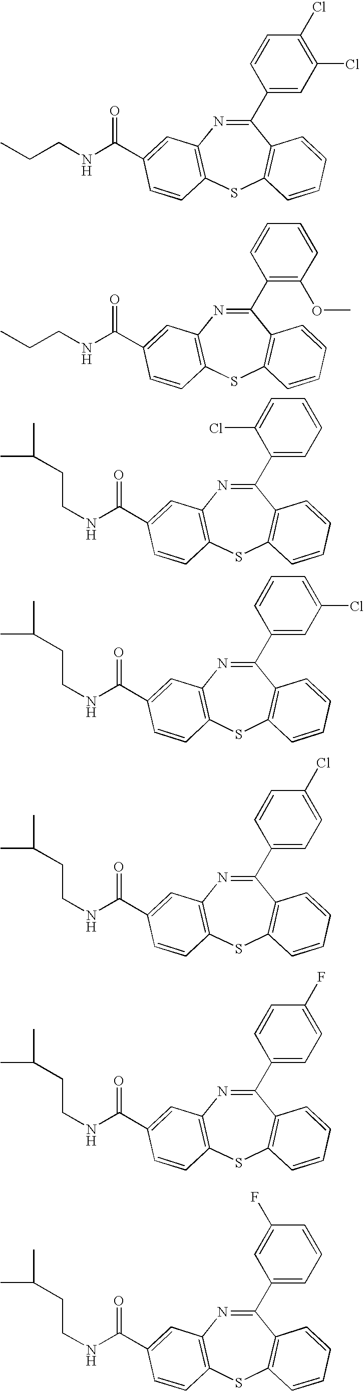

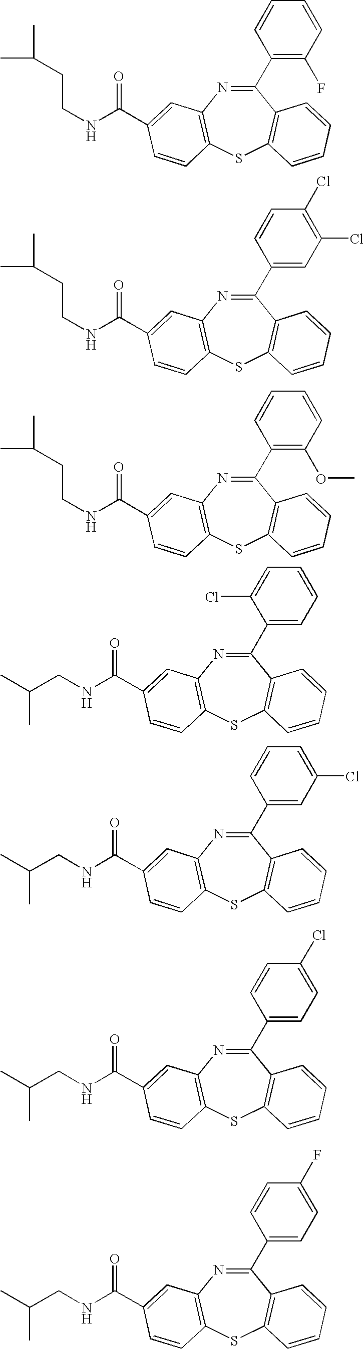

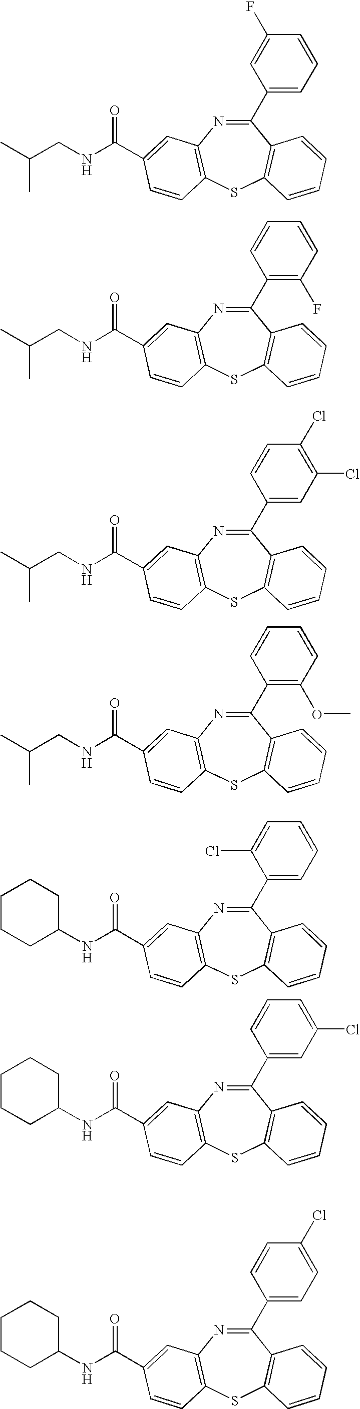

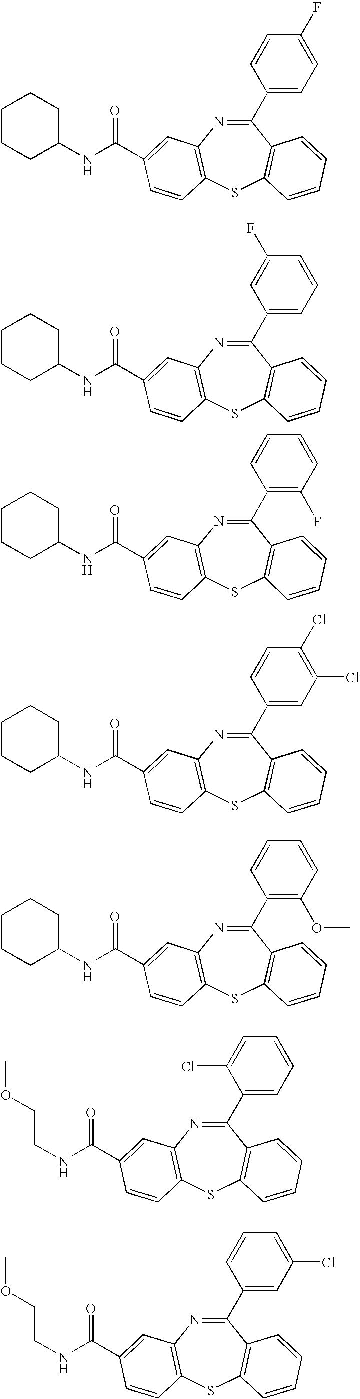

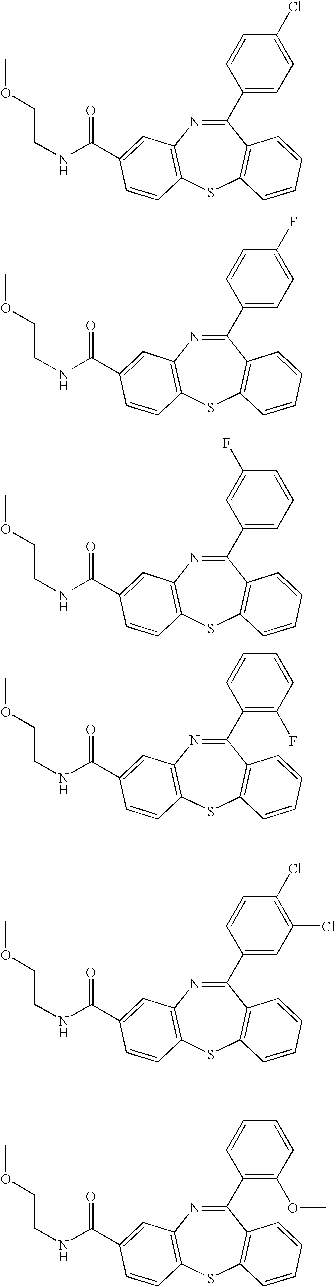

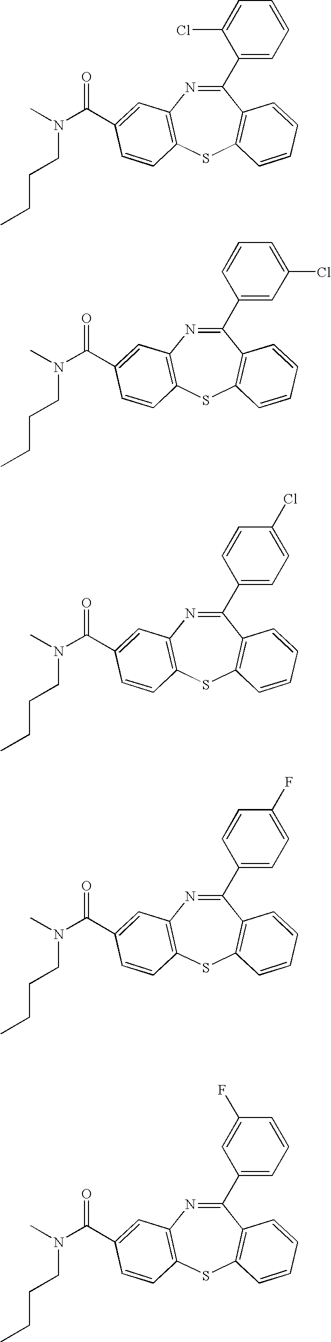

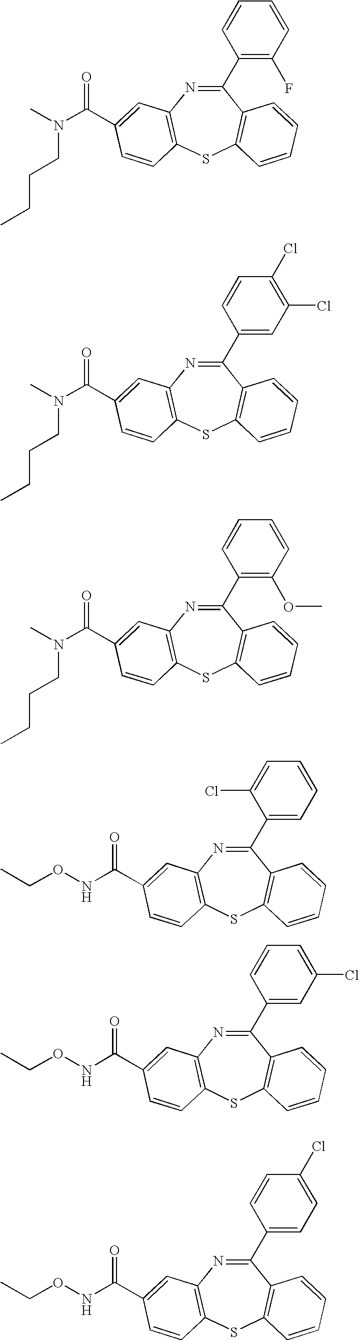

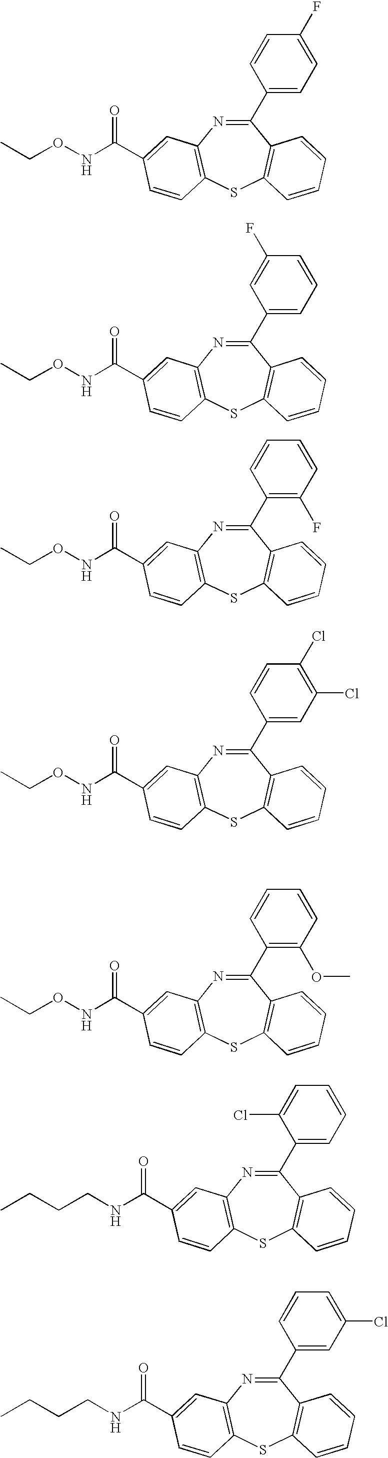

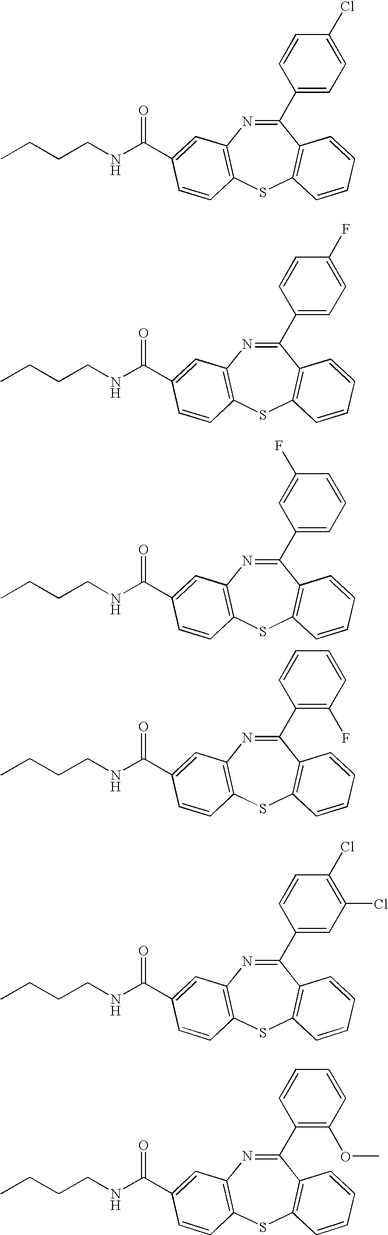

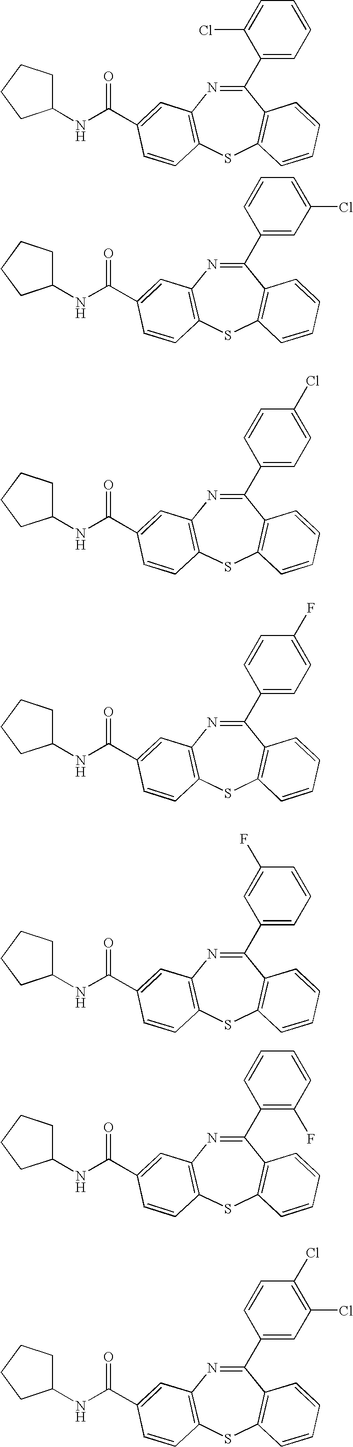

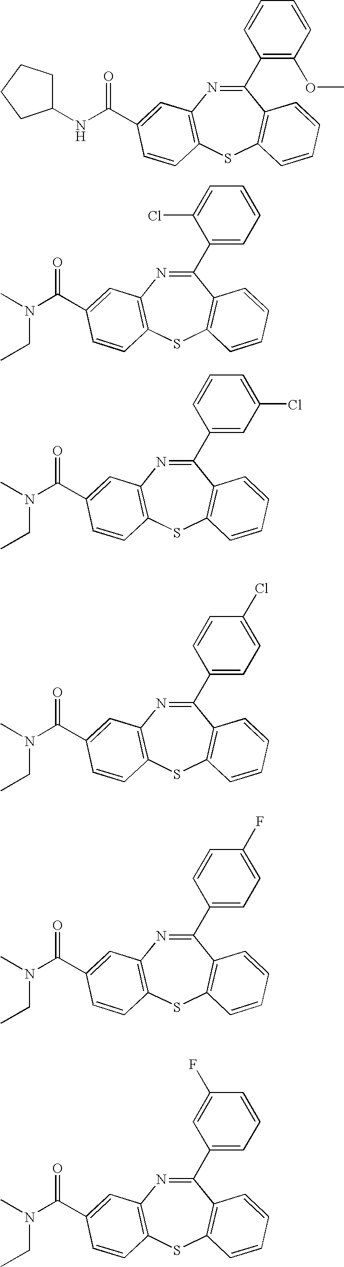

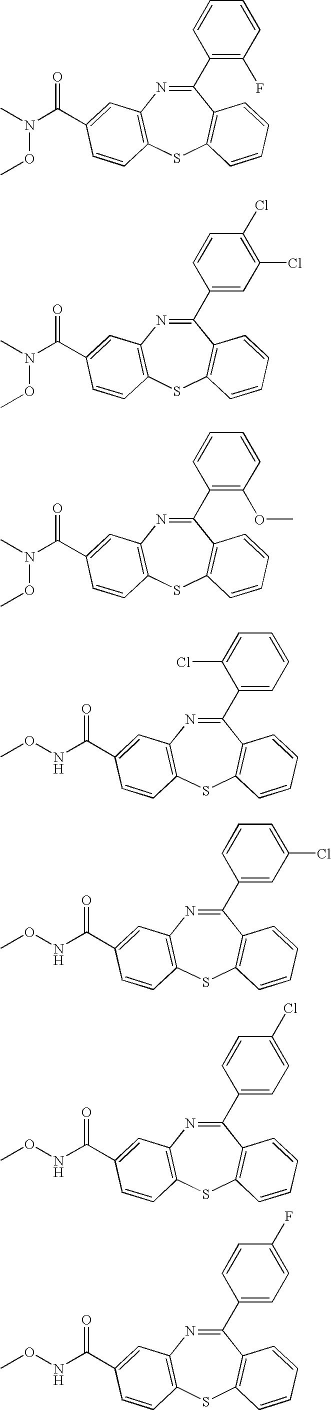

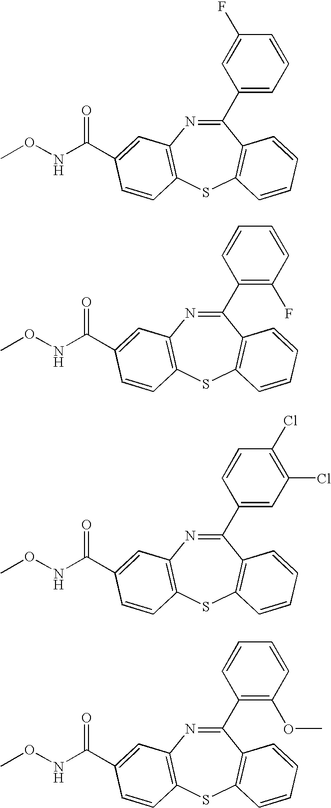

77. The compound of claim 1, wherein the compound is selected from the group consisting of:

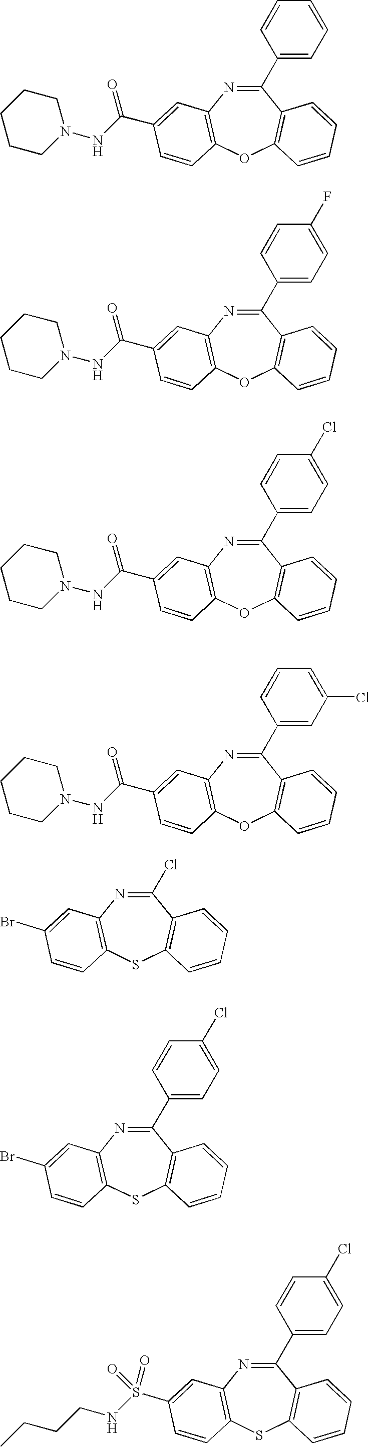

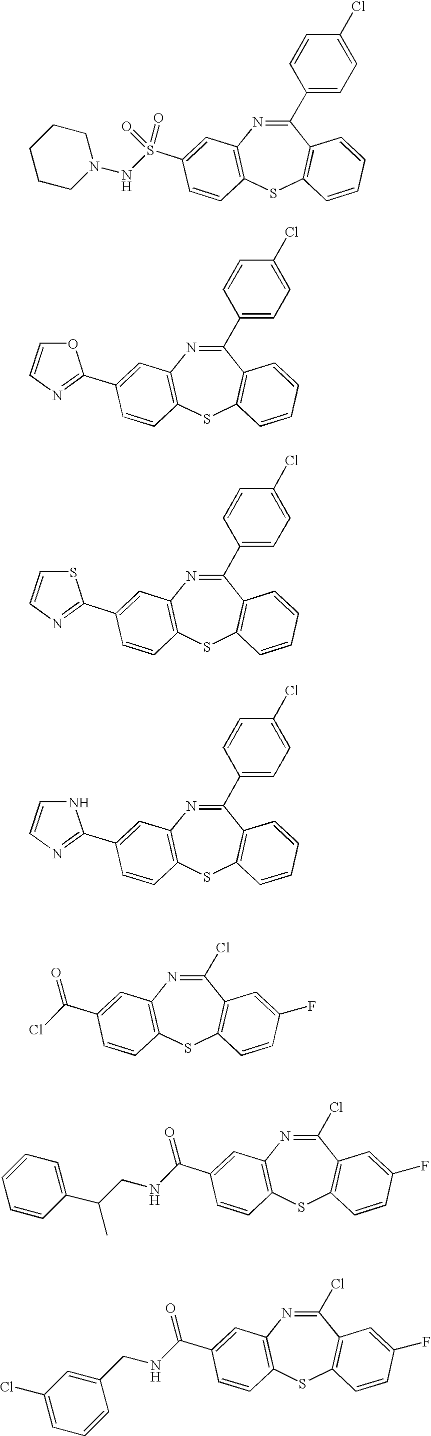

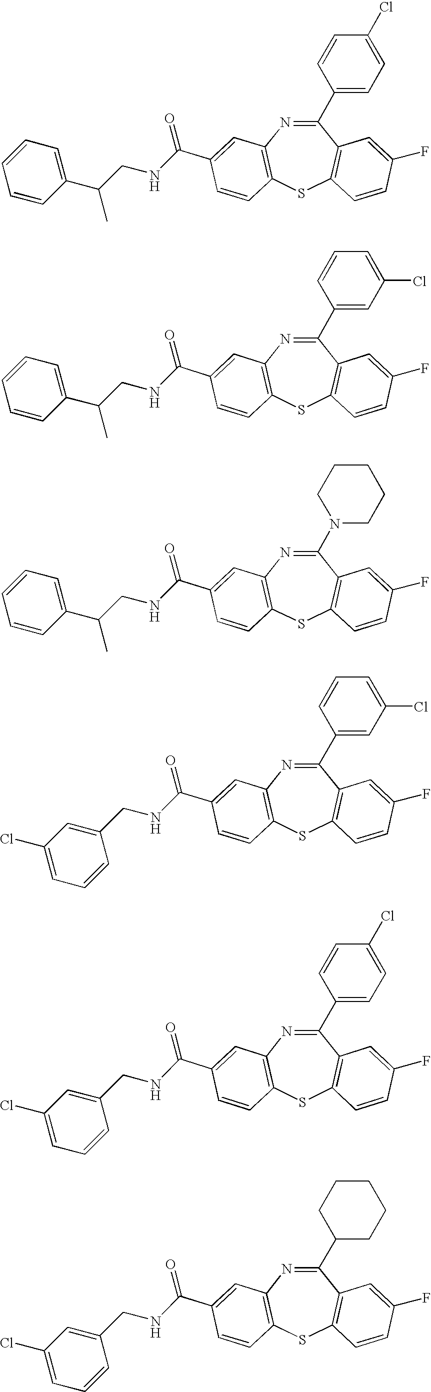

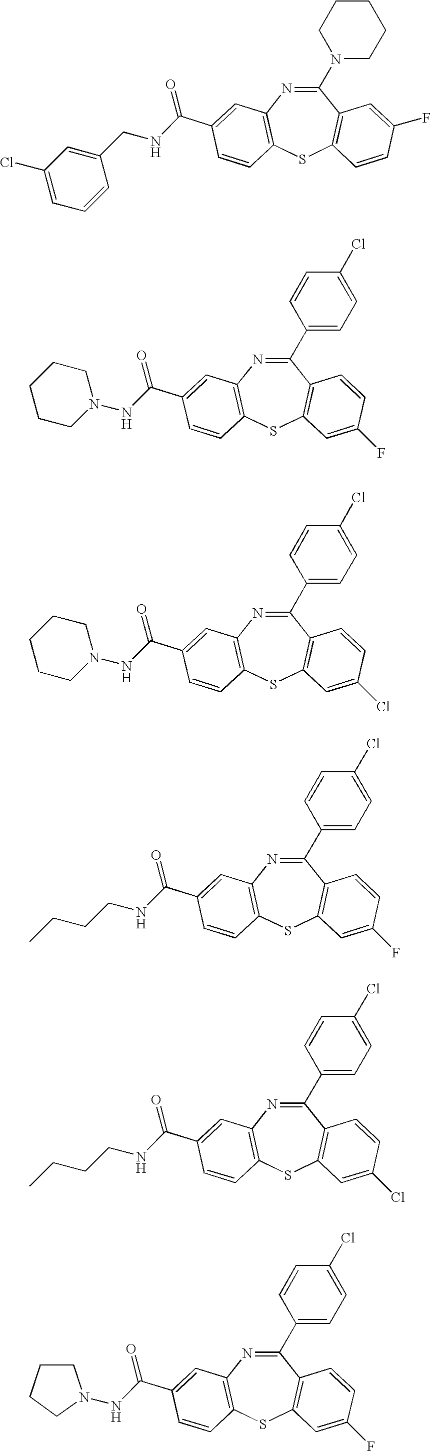

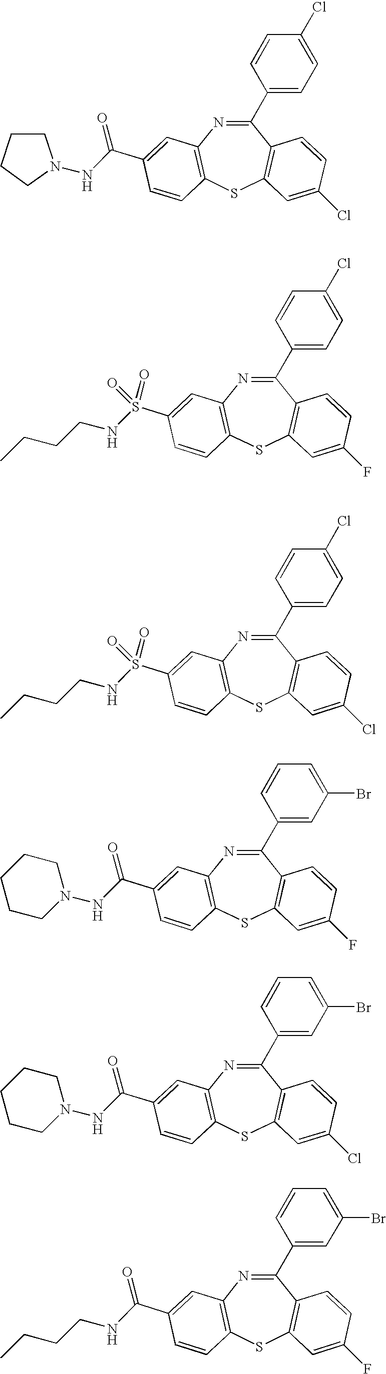

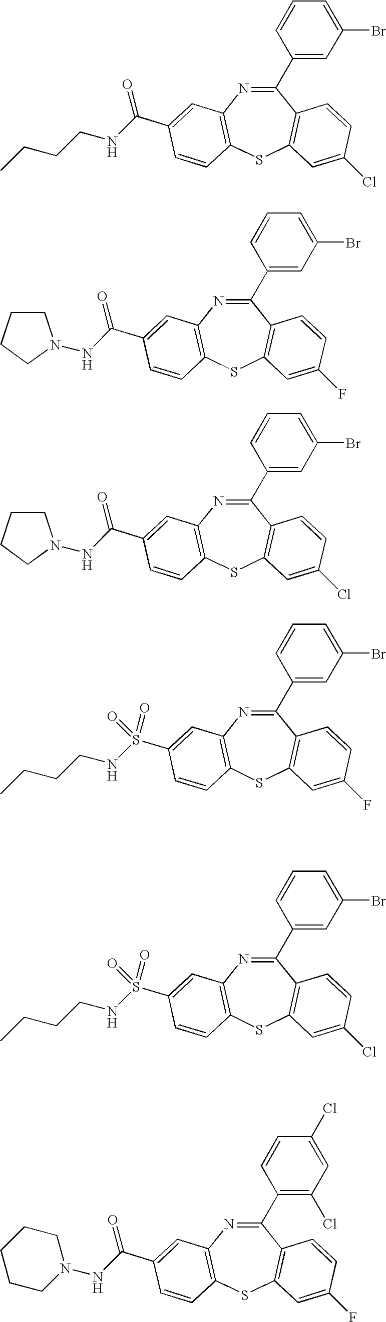

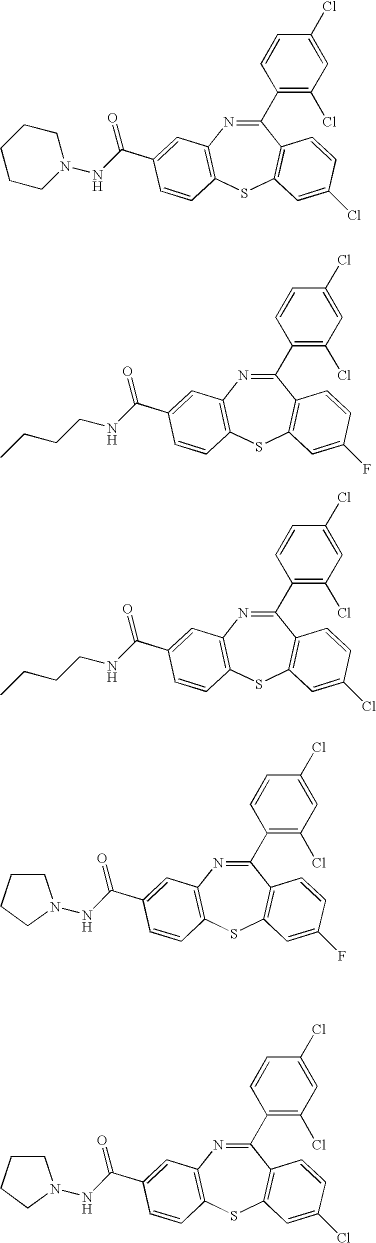

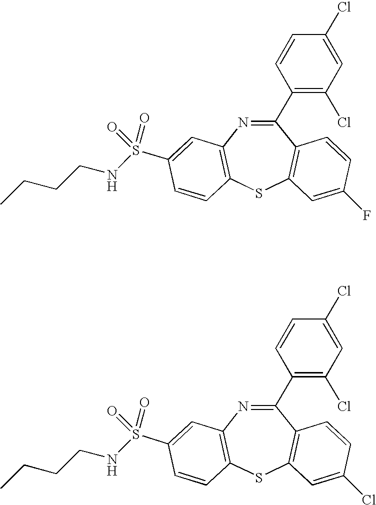

78. The compound of claim 1, wherein the compound is selected from the group consisting of:

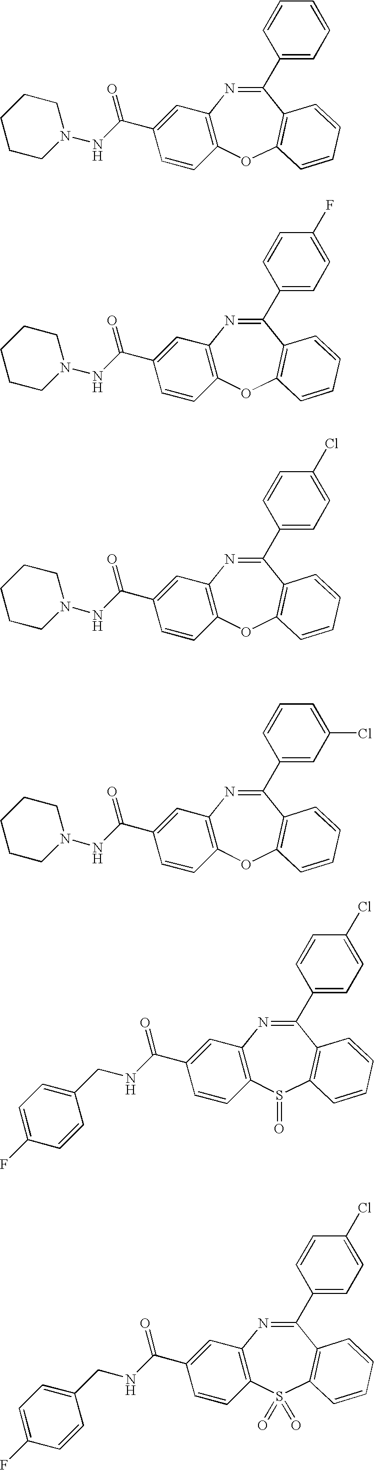

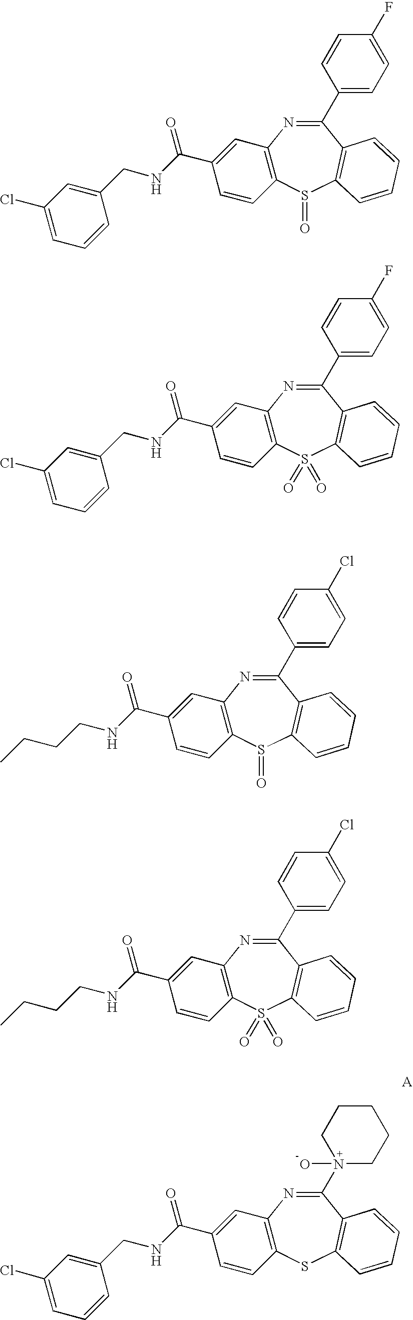

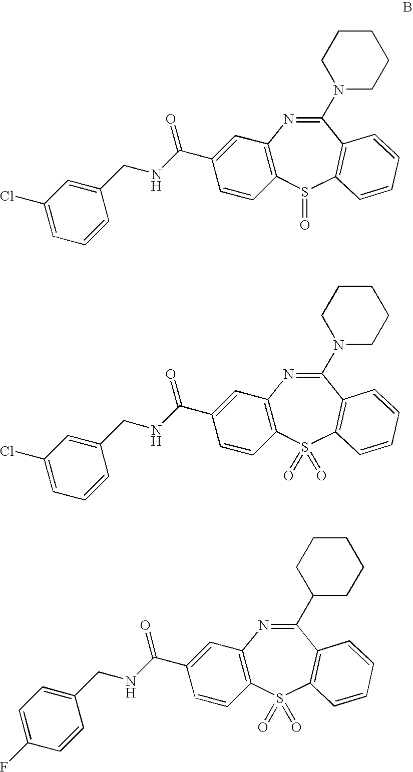

79. The compound of claim 1, wherein the compound is selected from the group consisting of:

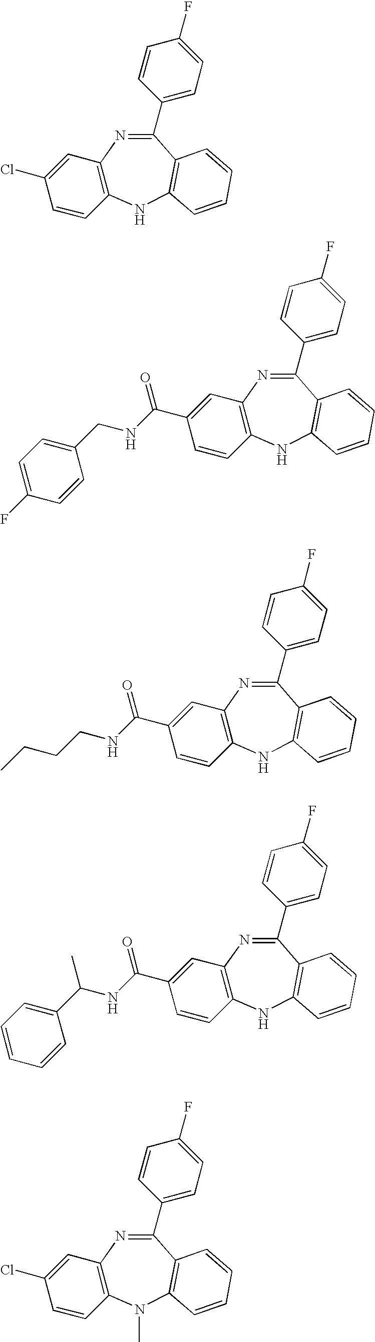

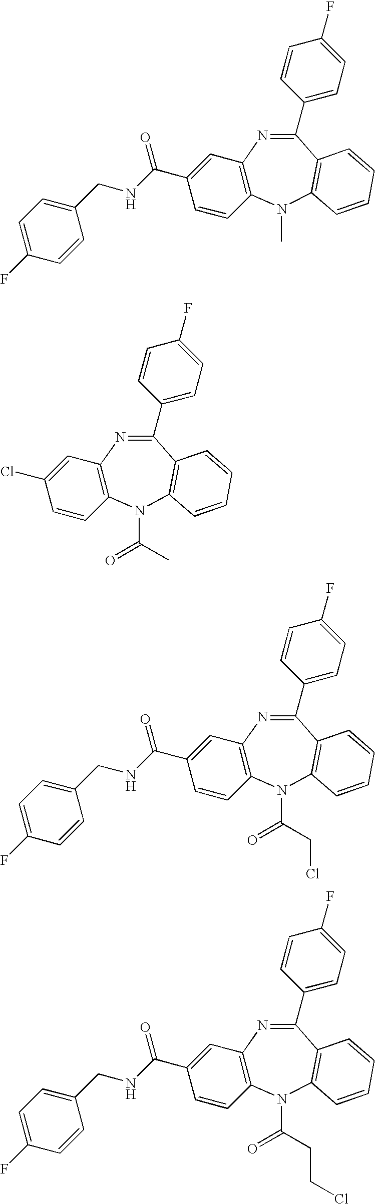

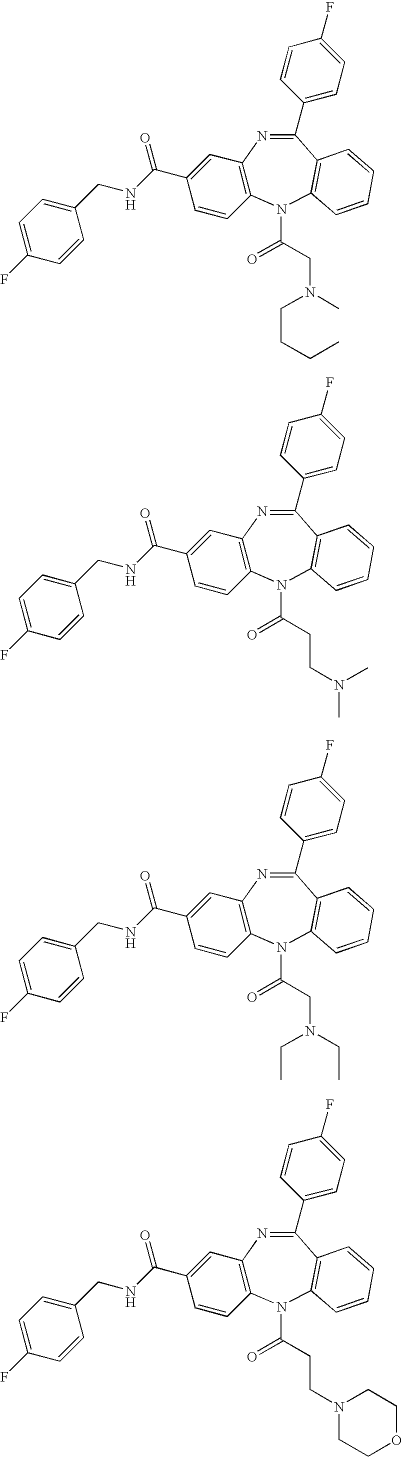

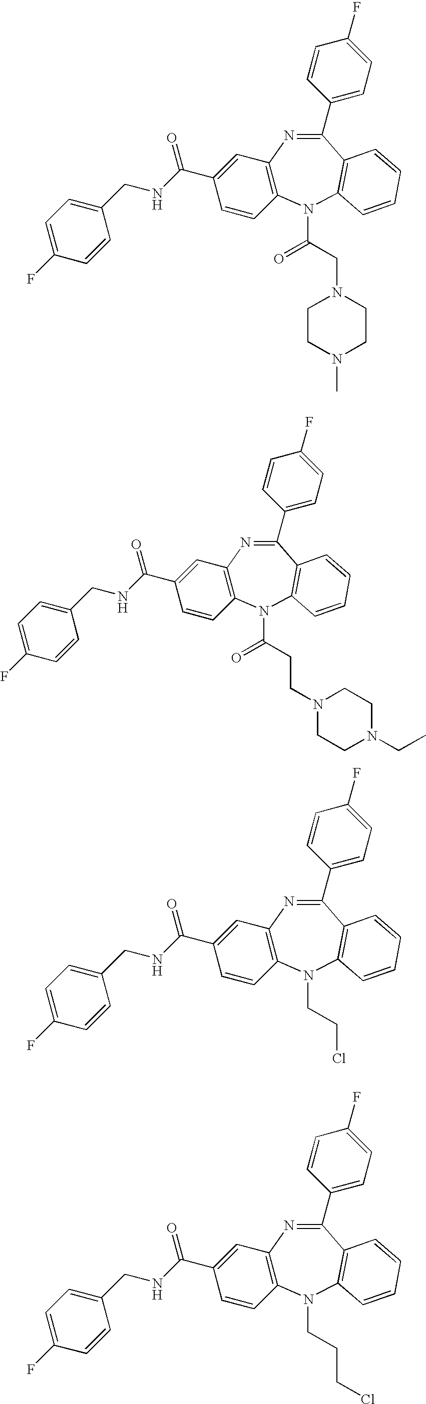

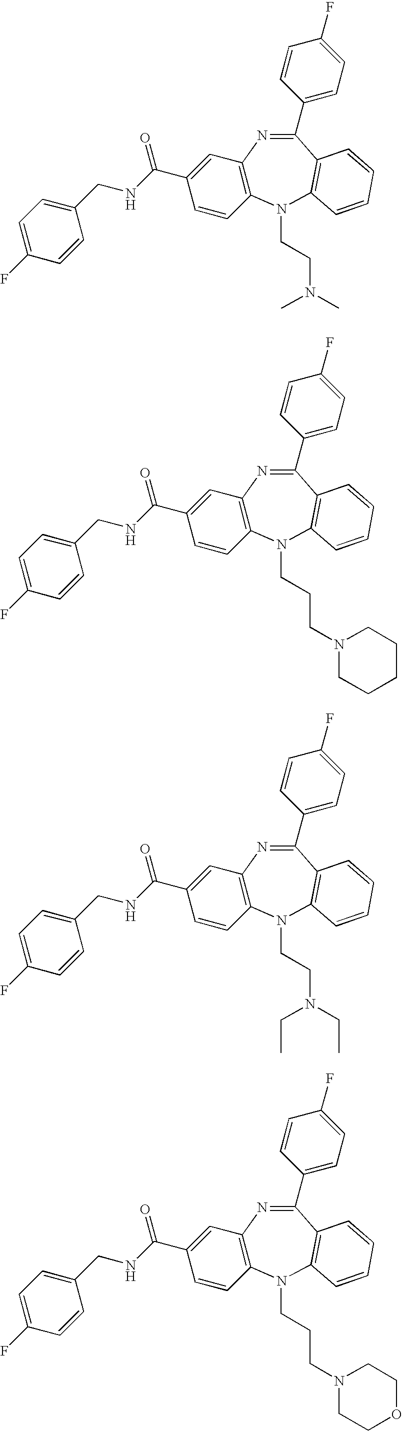

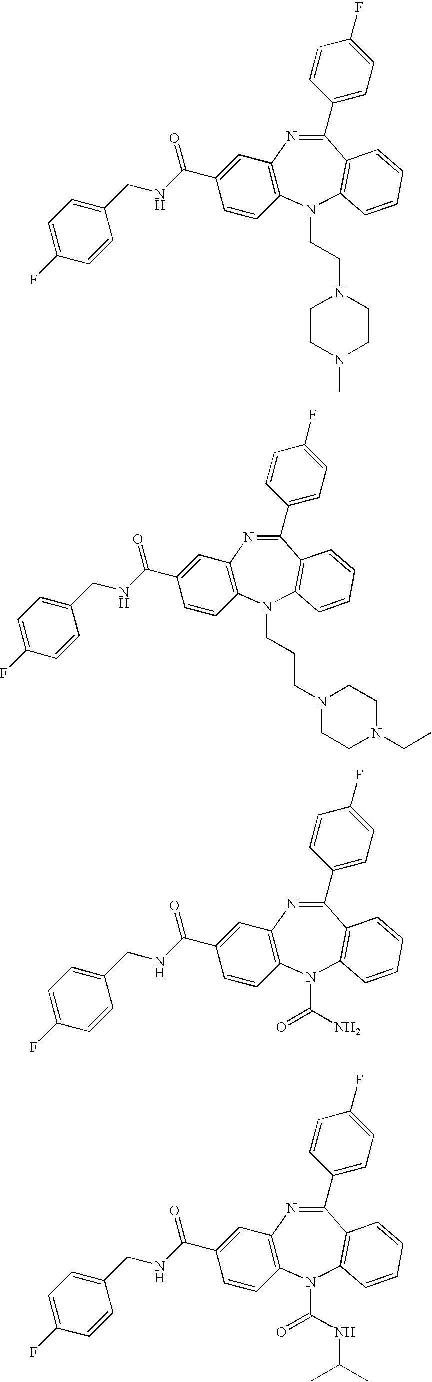

80. The compound of claim 1, wherein the compound is selected from the group consisting of:

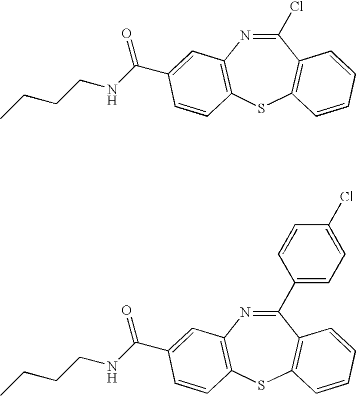

81. The compound of claim 1, wherein the compound is selected from the group consisting of:

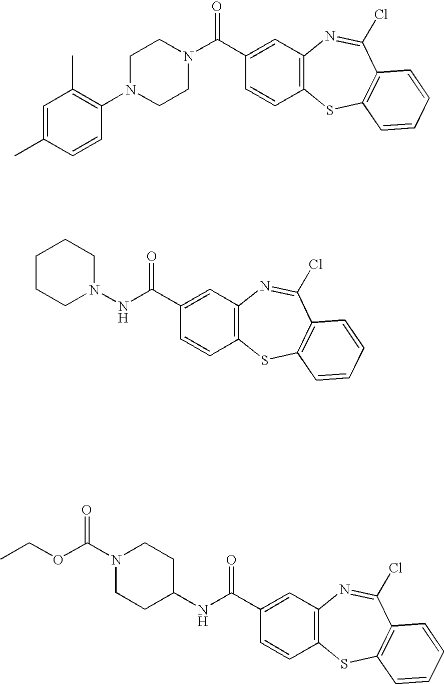

82. The compound of claim 1, wherein the compound is selected from the group consisting of:

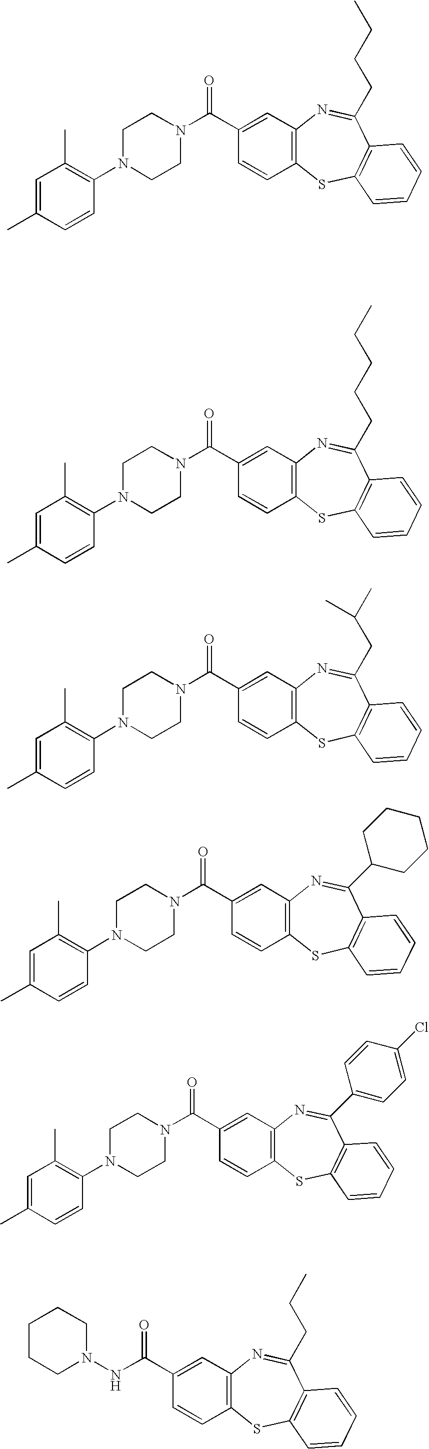

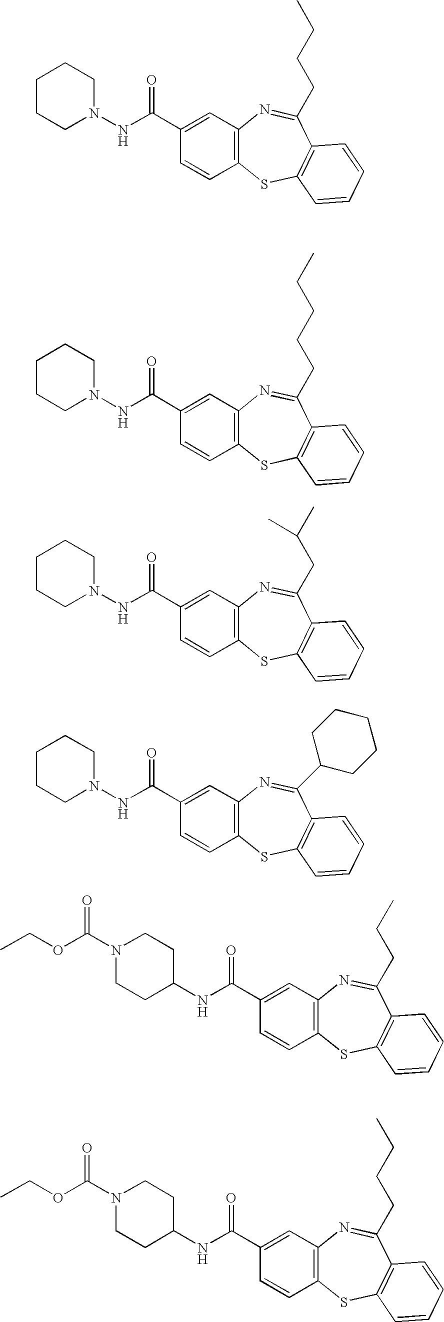

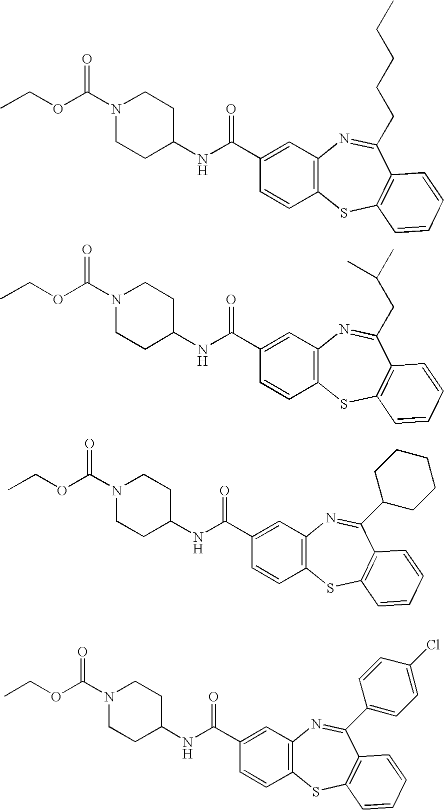

83. The compound of claim 1, wherein the compound is selected from the group consisting of:

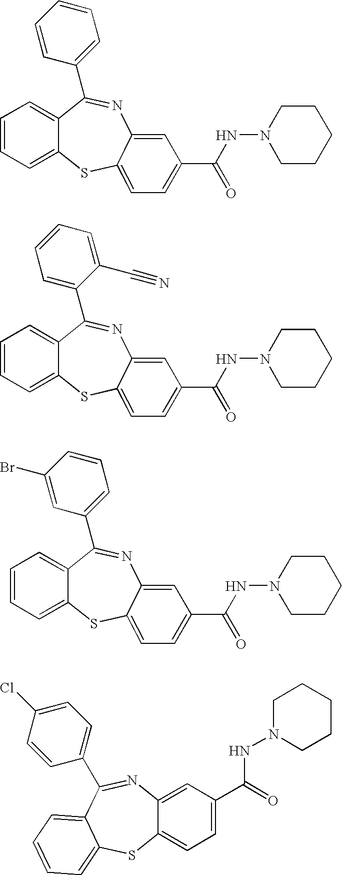

84. The compound of claim 1, wherein the compound is selected from the group consisting of:

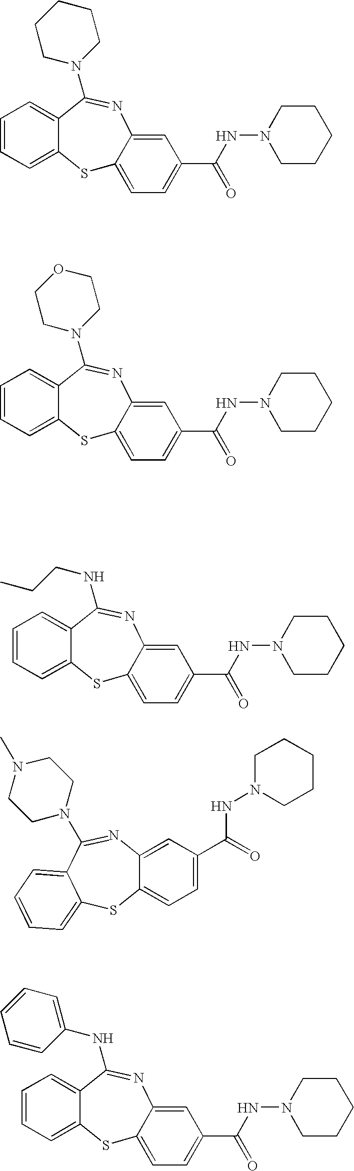

85. The compound of claim 1, wherein the compound is selected from the group consisting of:

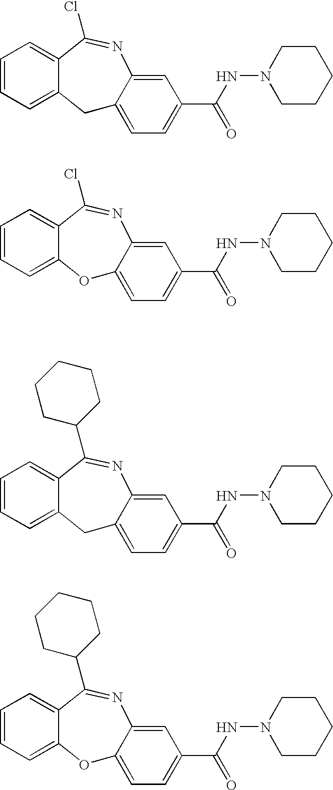

86. The compound of claim 1, wherein the compound is selected from the group consisting of:

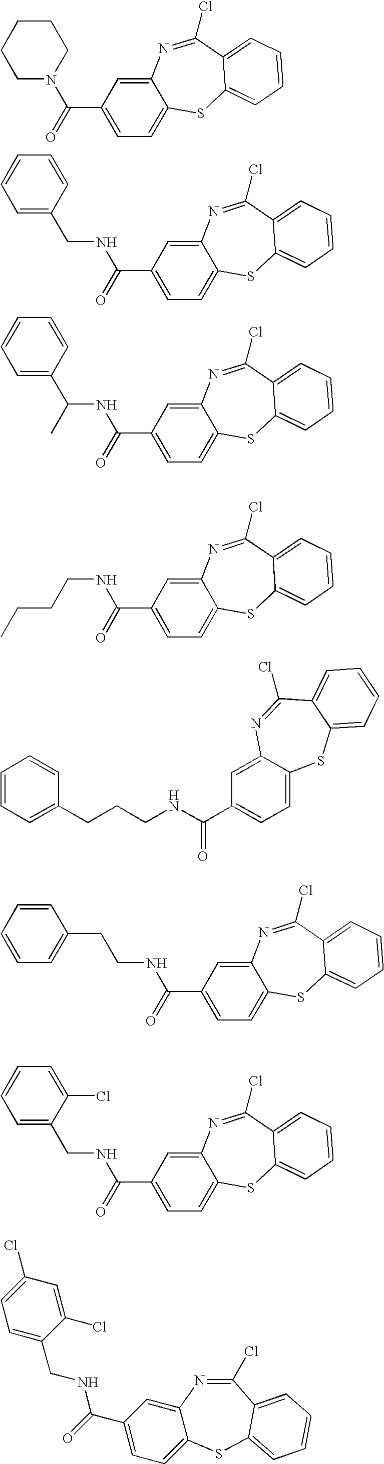

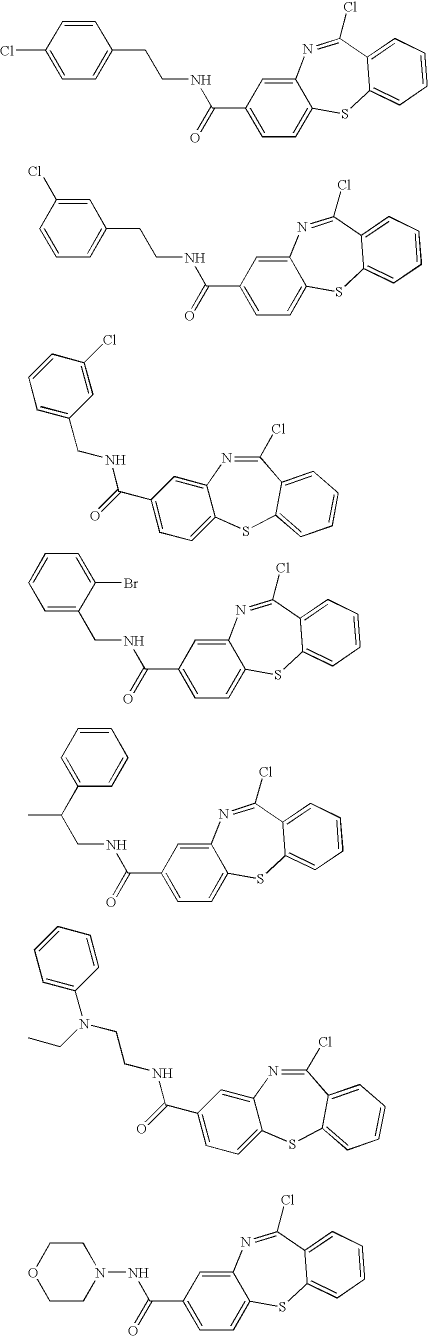

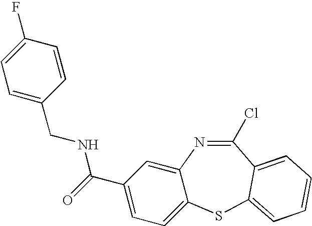

87. The compound of claim 1, wherein the compound is selected from the group consisting of:

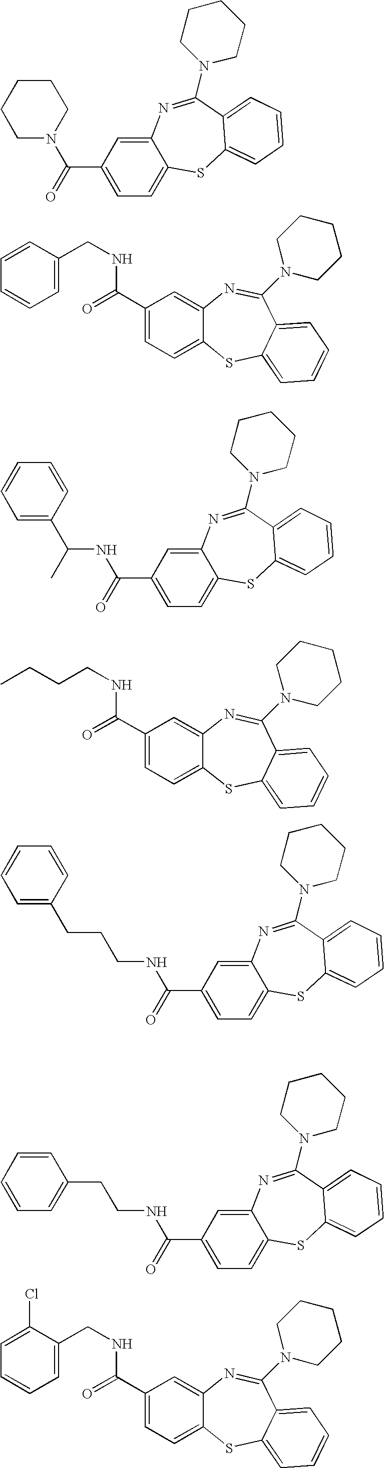

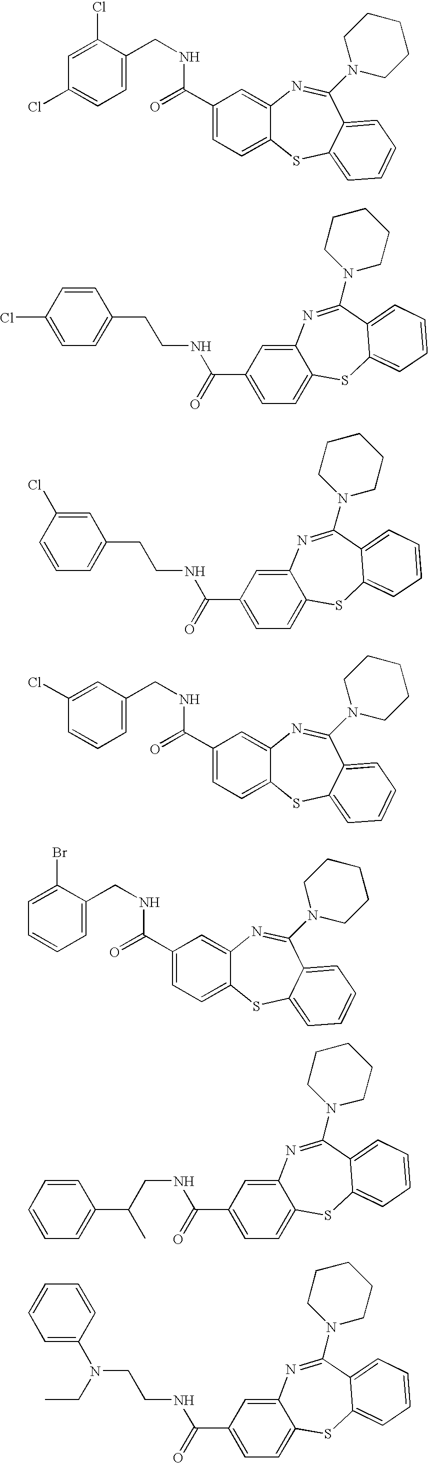

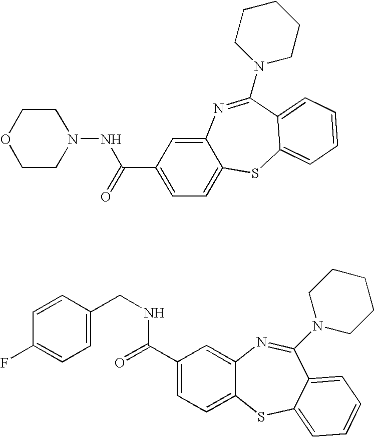

88. The compound of claim 1, wherein the compound is selected from the group consisting of:

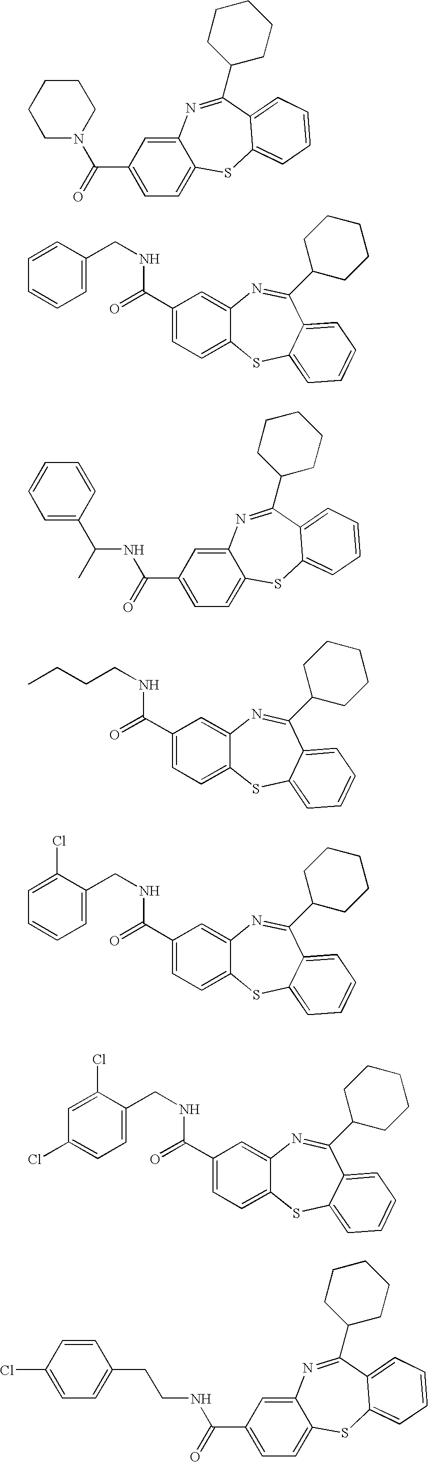

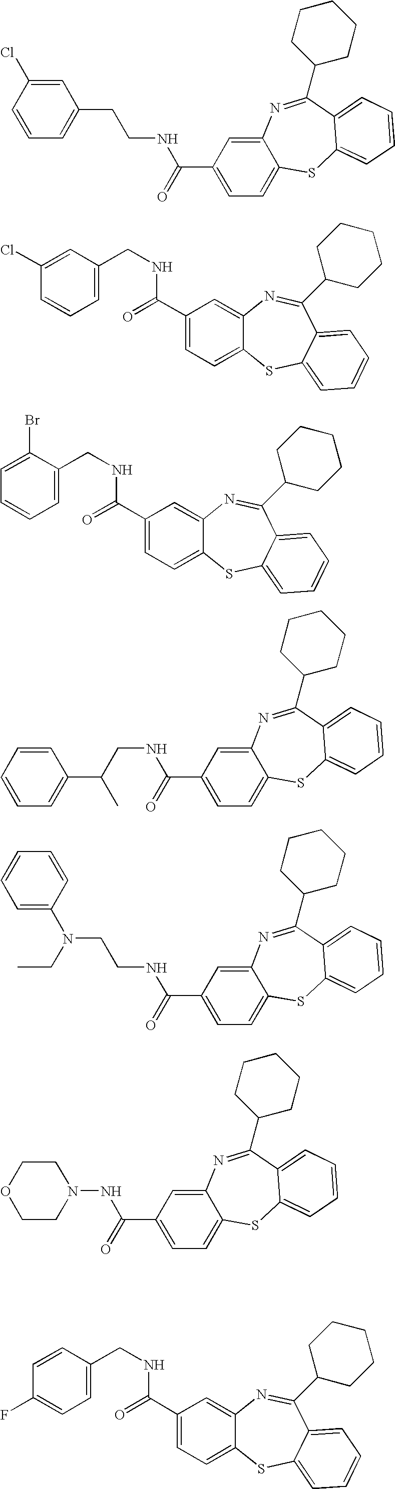

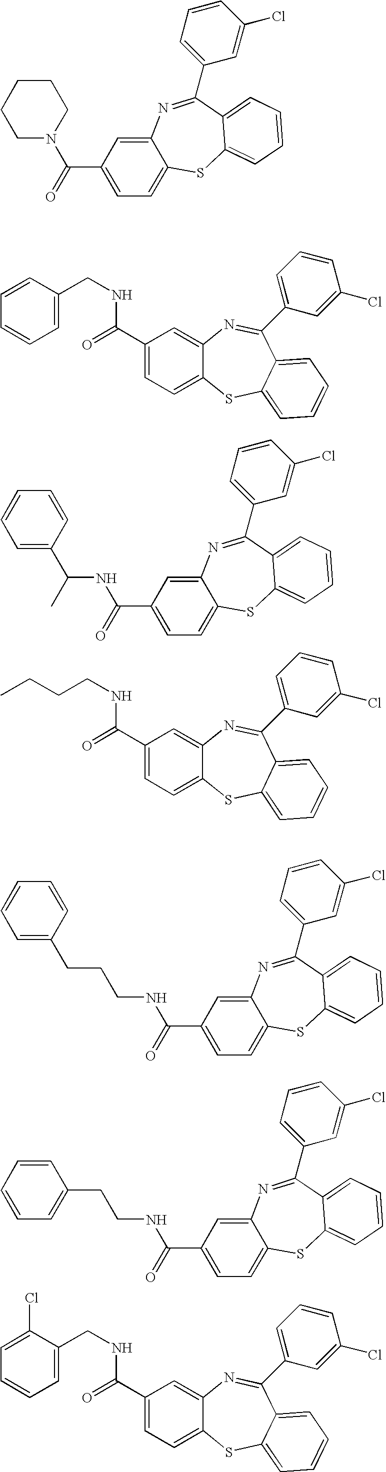

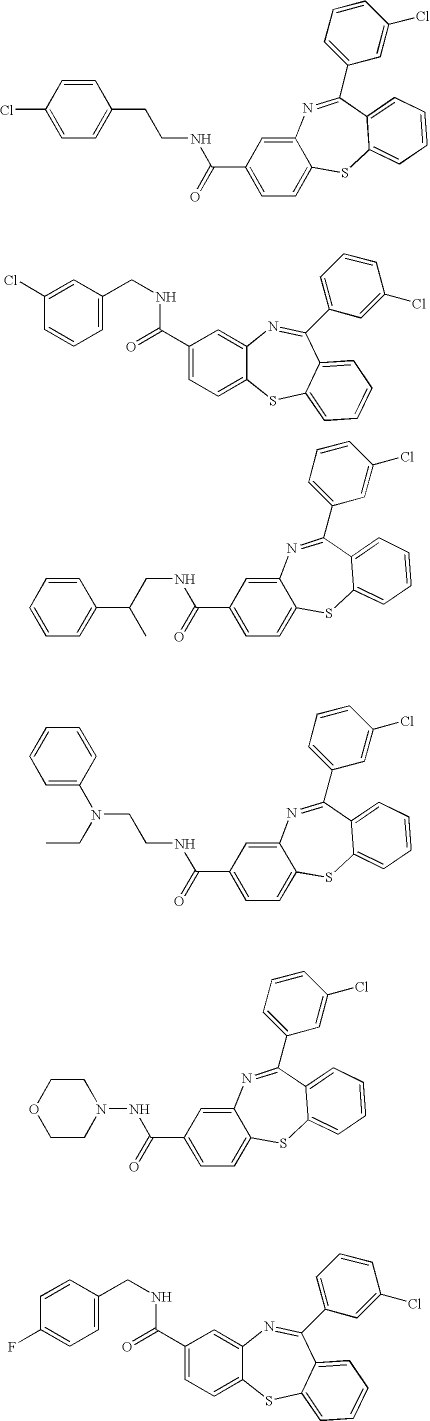

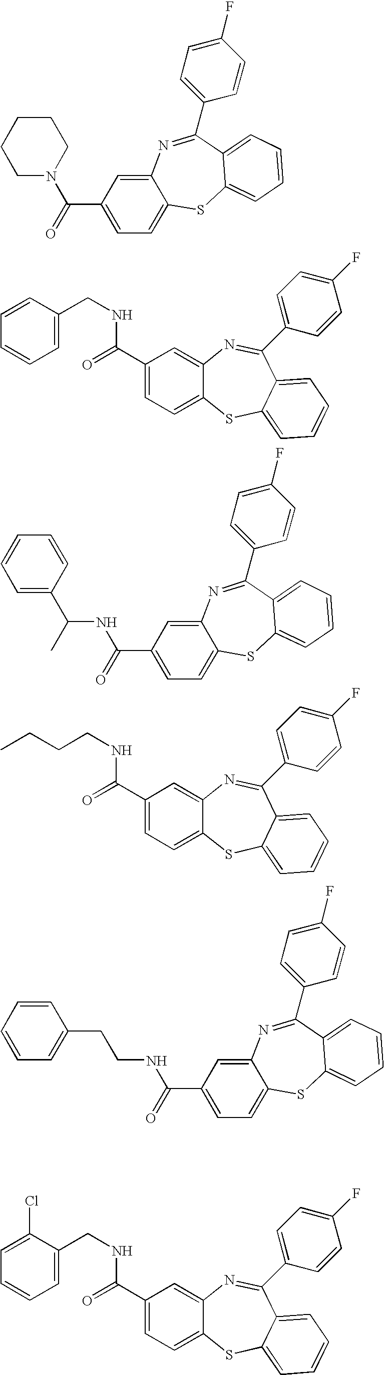

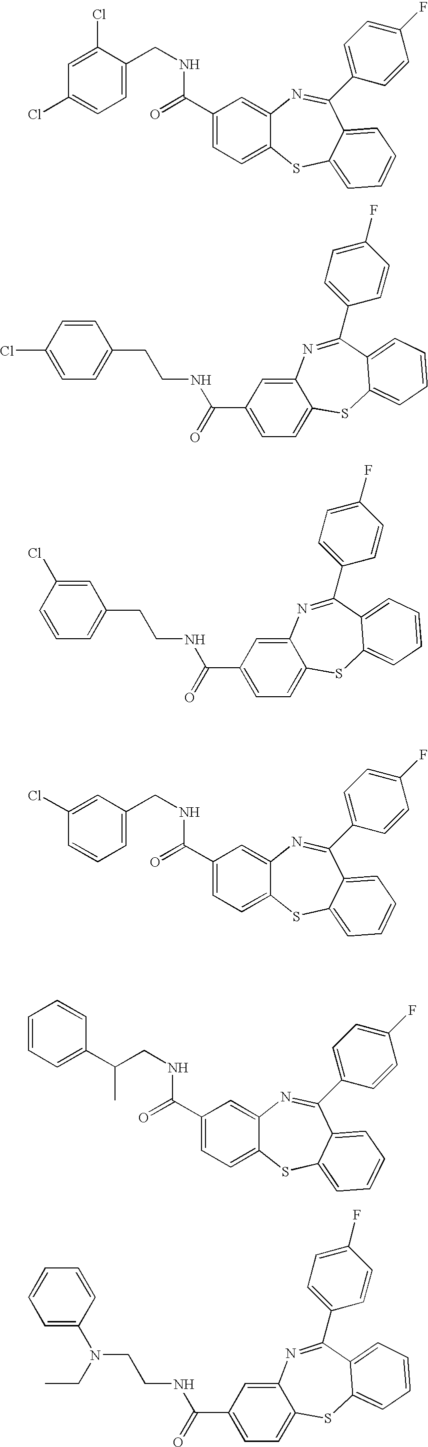

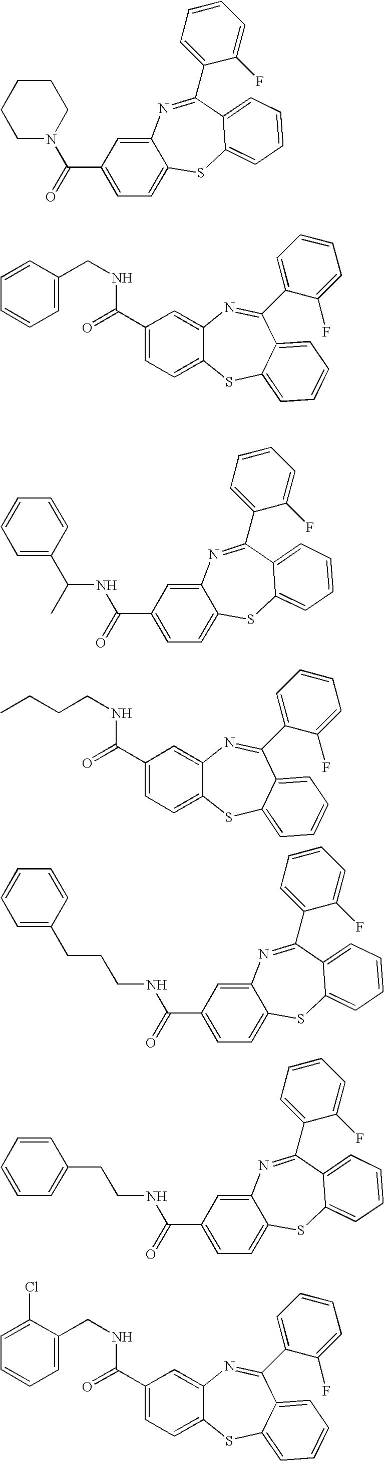

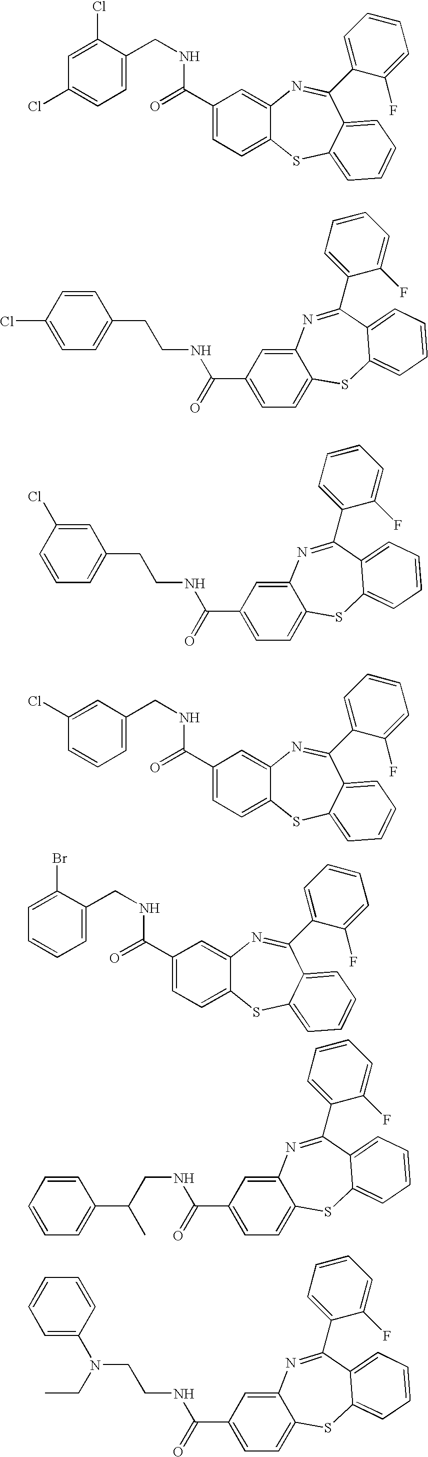

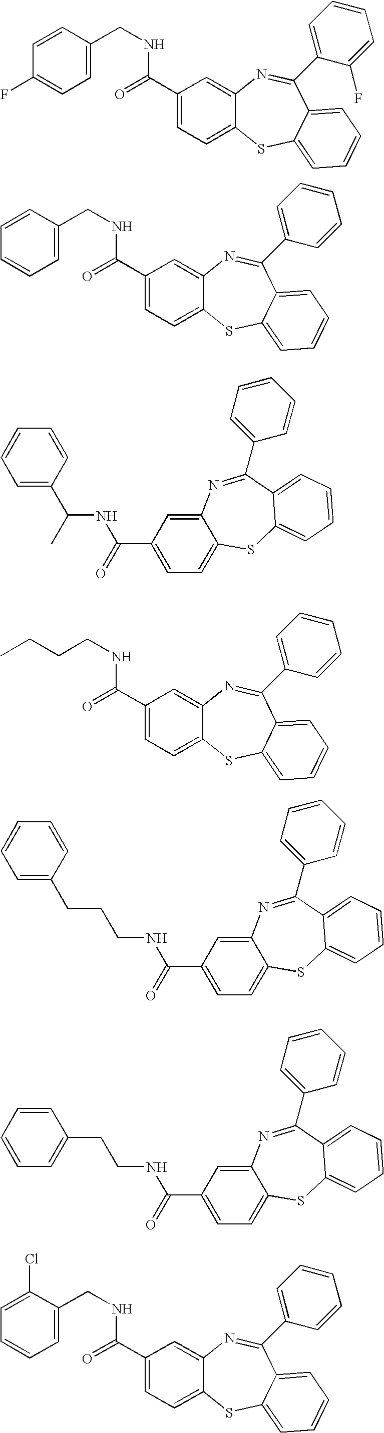

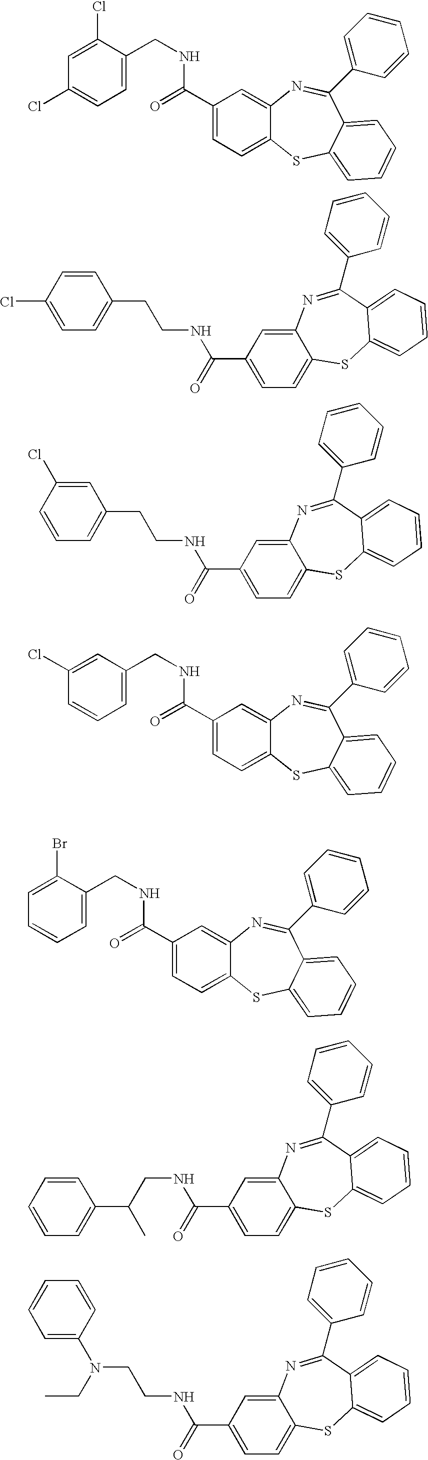

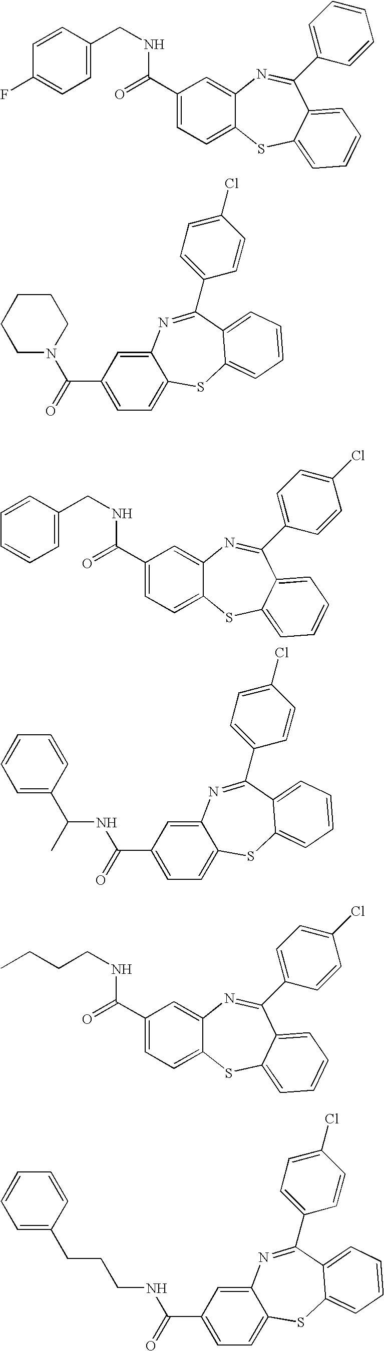

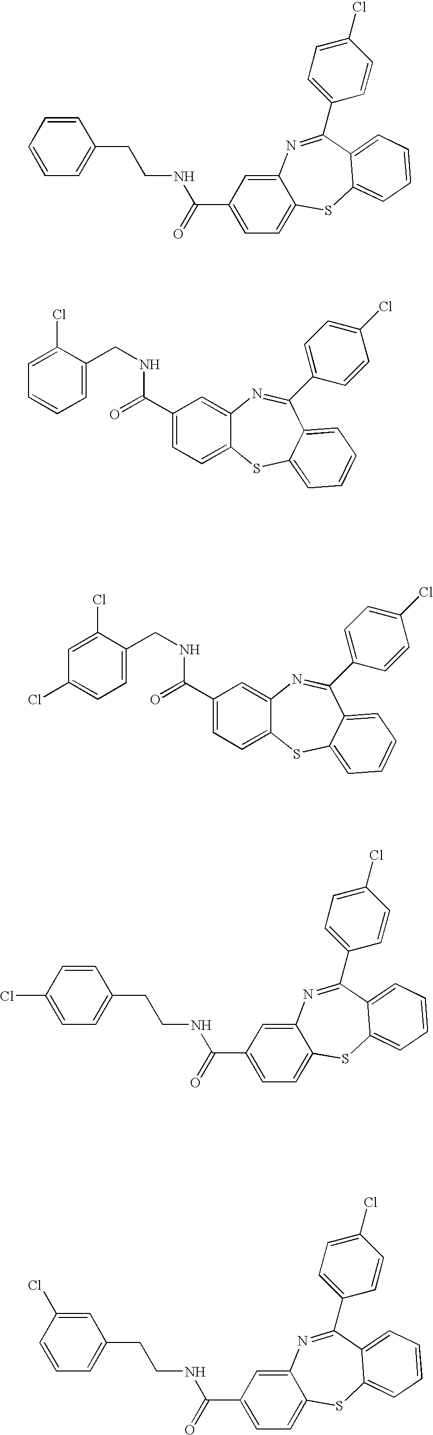

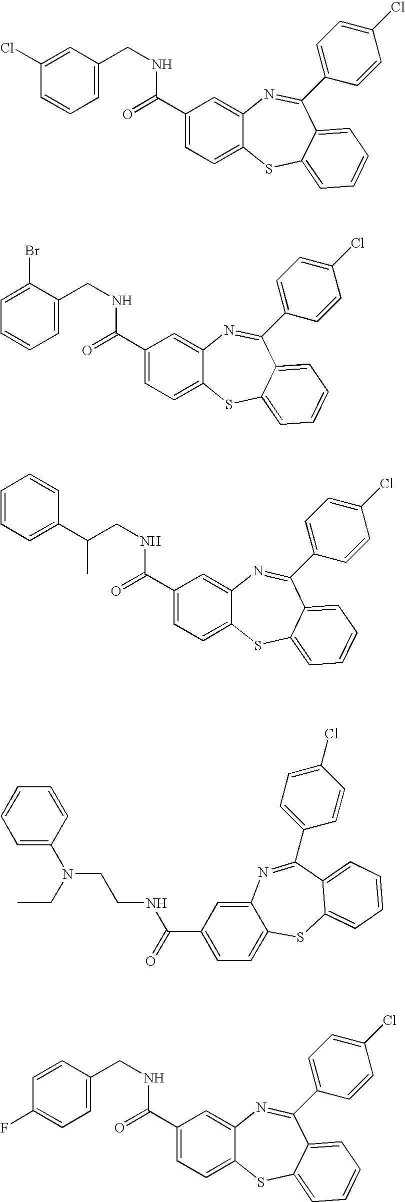

89. The compound of claim 1, wherein the compound is selected from the group consisting of:

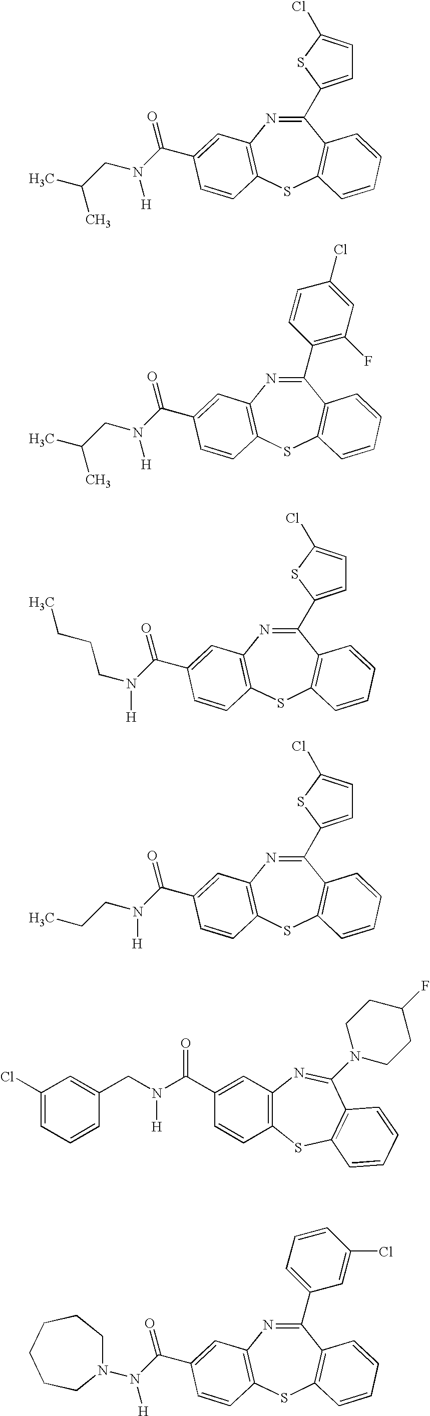

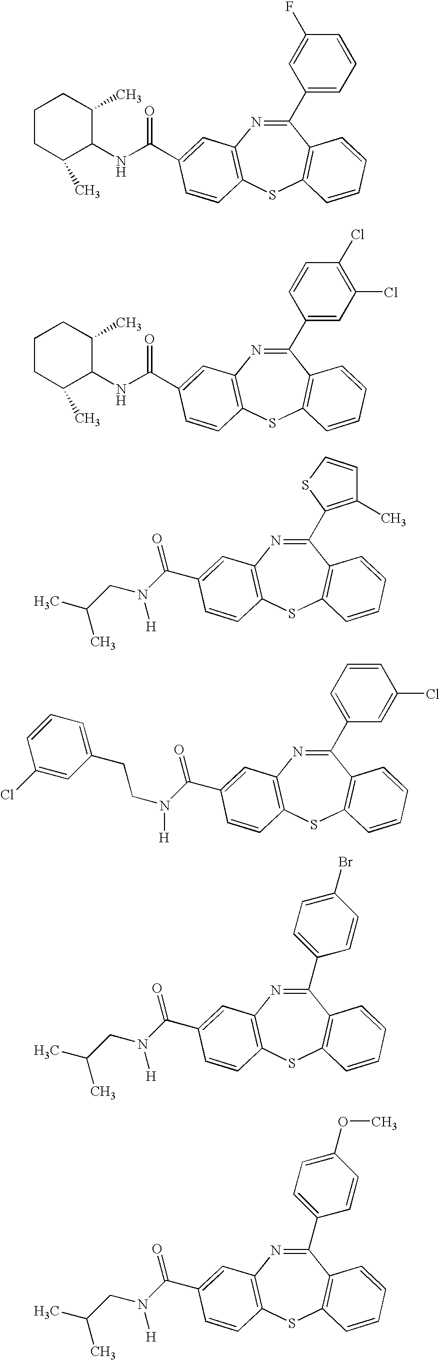

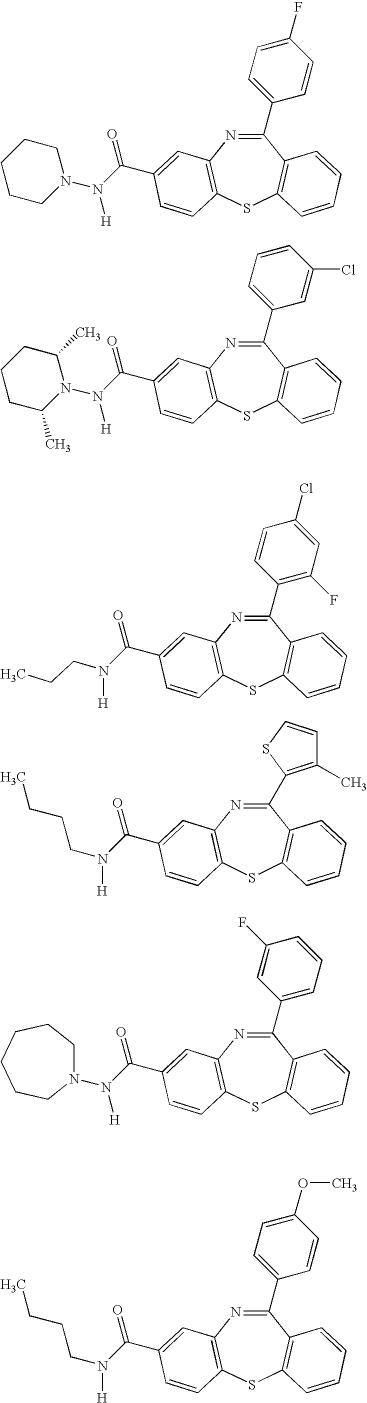

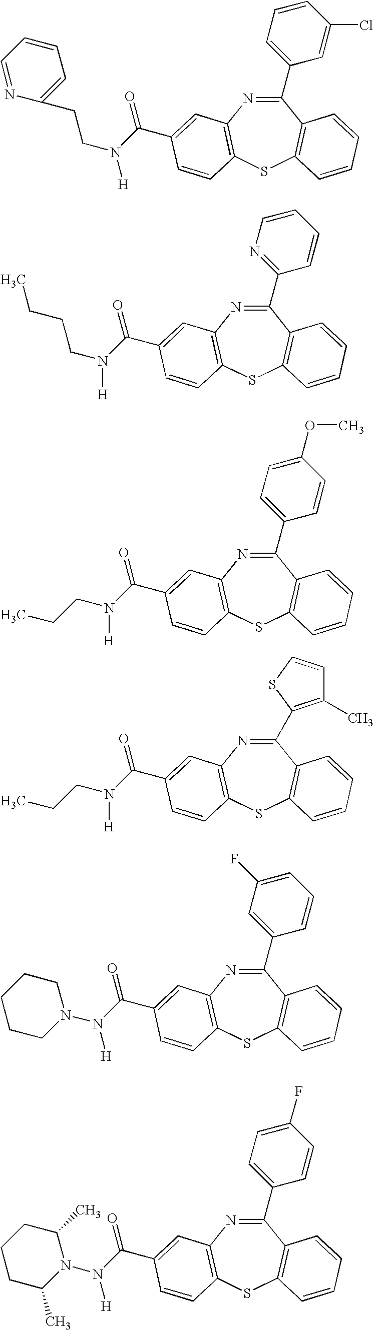

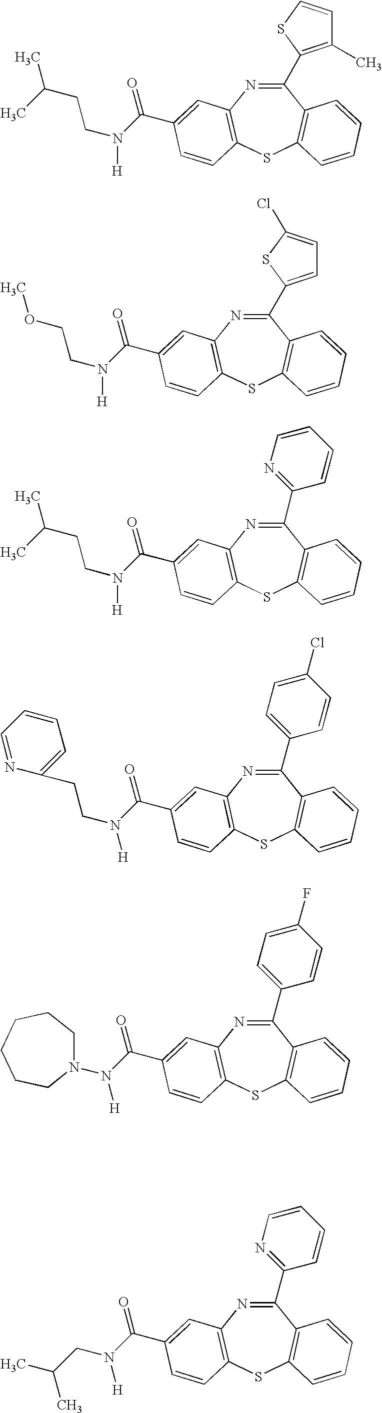

90. The compound of claim 1, wherein the compound is selected from the group consisting of:

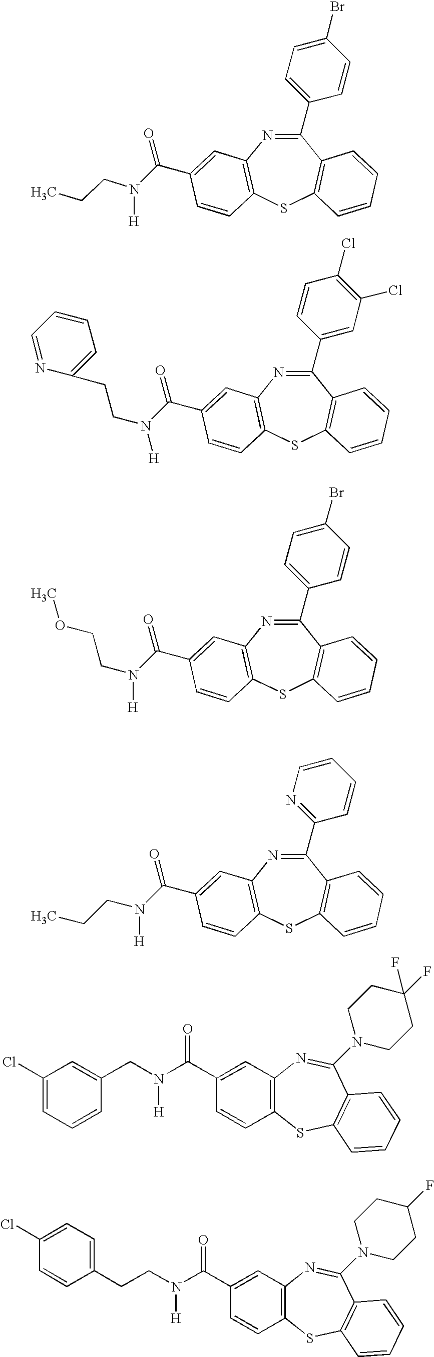

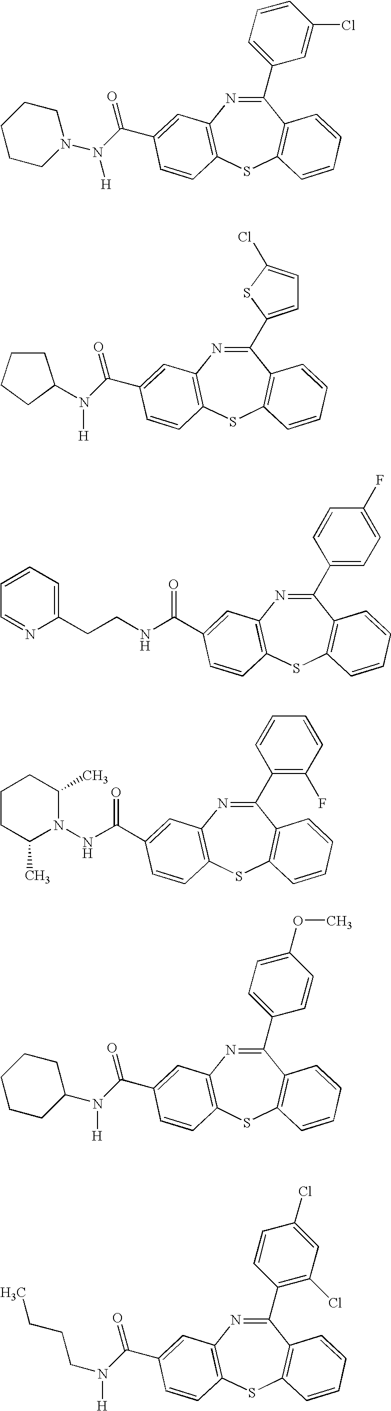

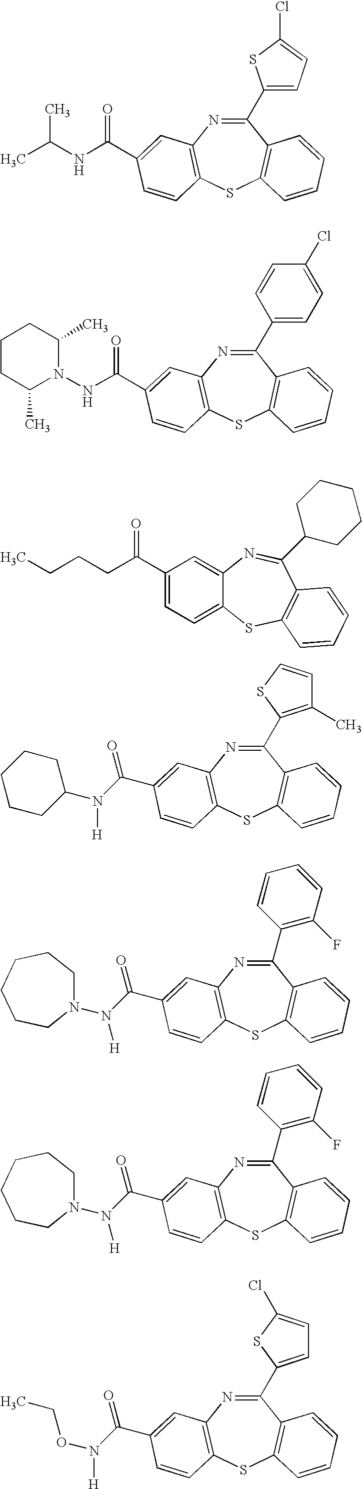

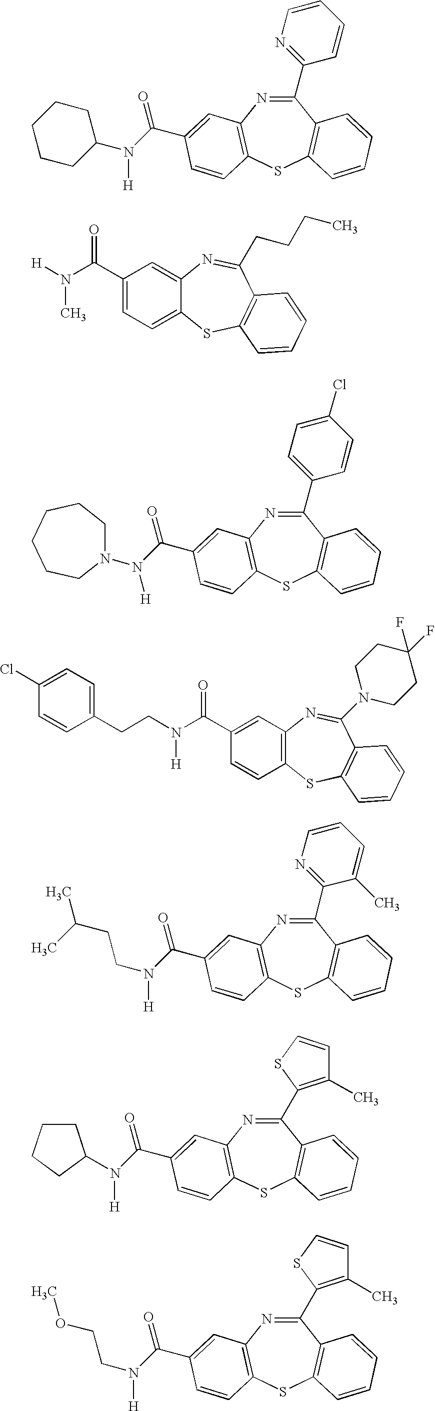

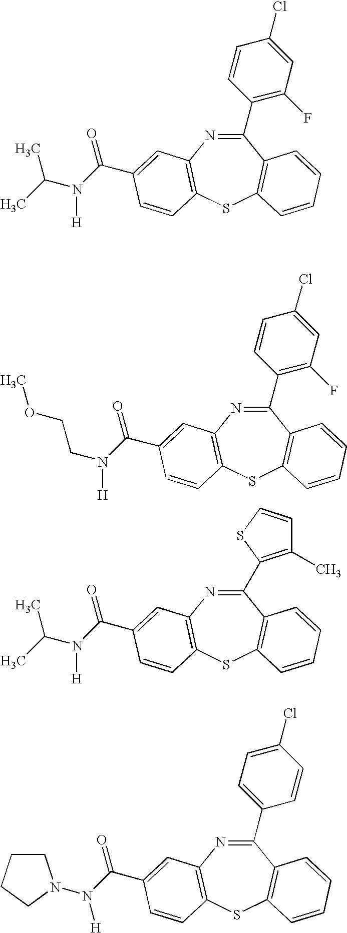

91. The compound of claim 1, wherein the compound is selected from the group consisting of:

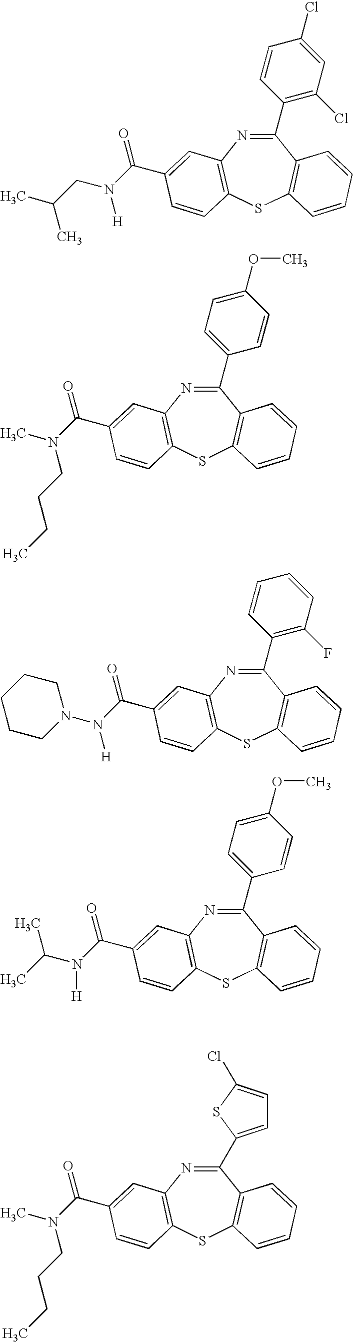

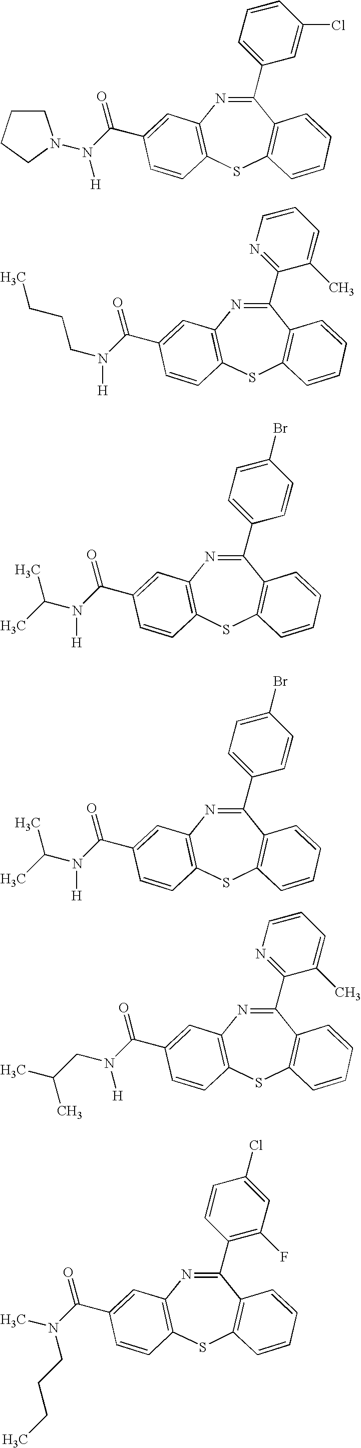

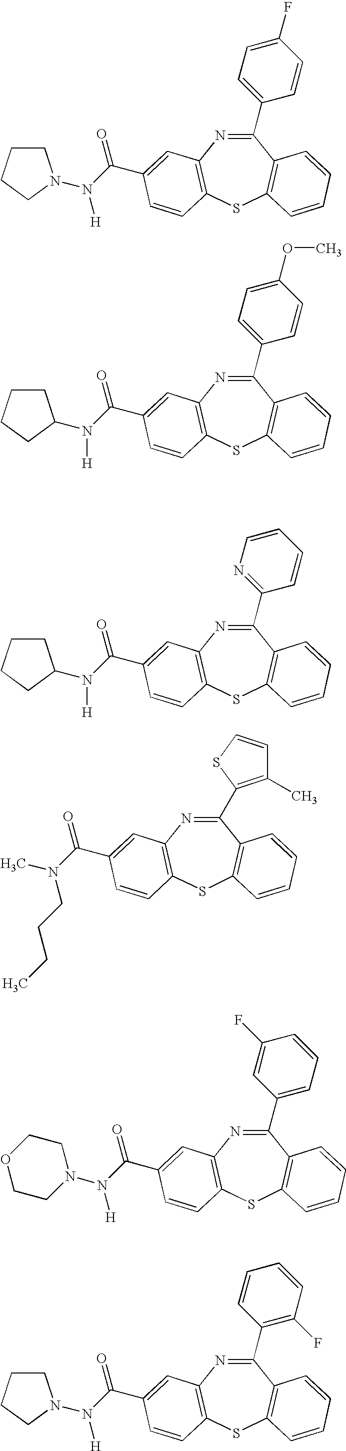

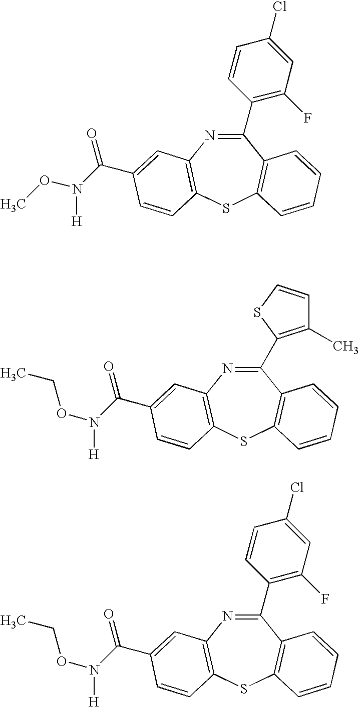

92. The compound of claim 1, wherein the compound is selected from the group consisting of:

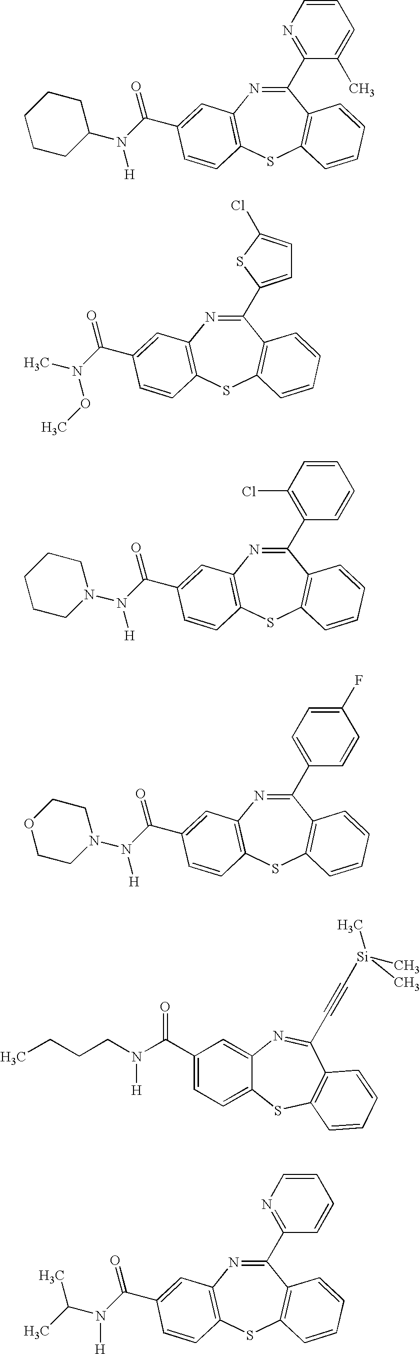

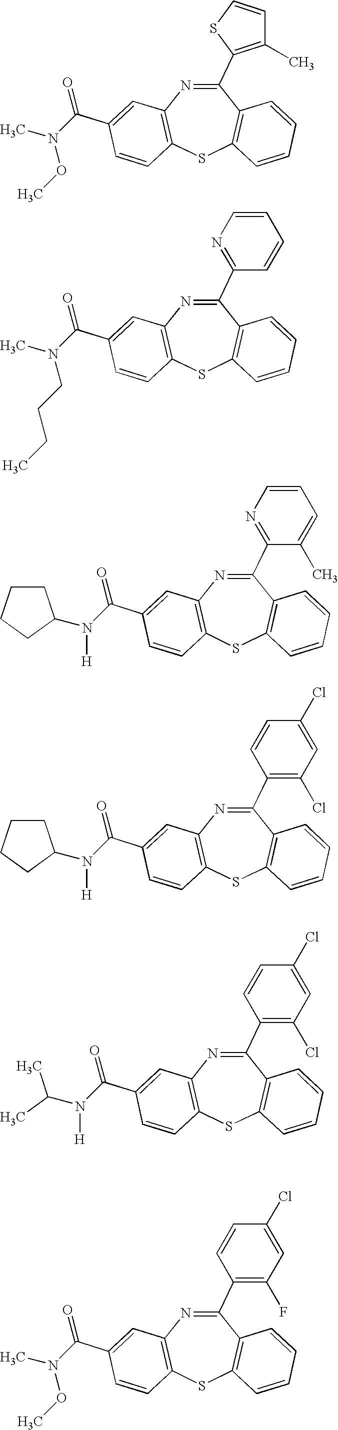

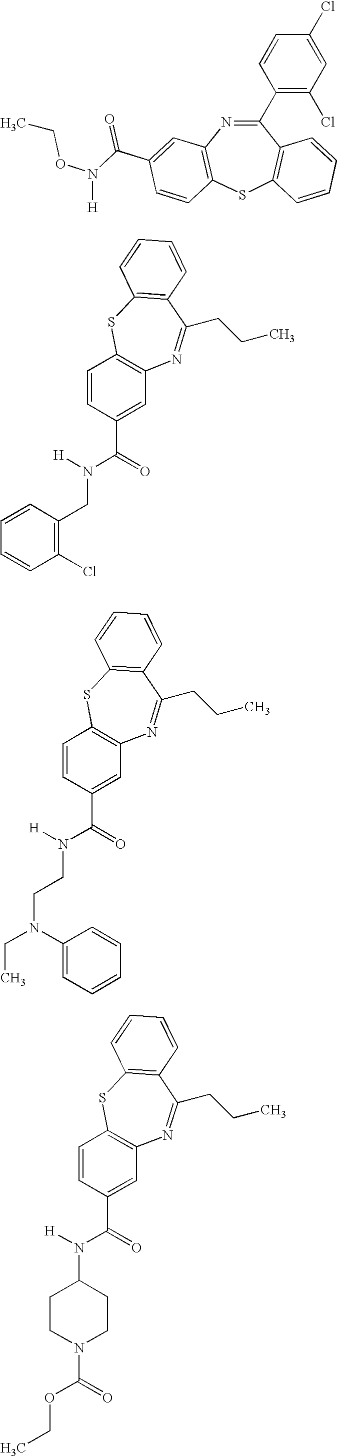

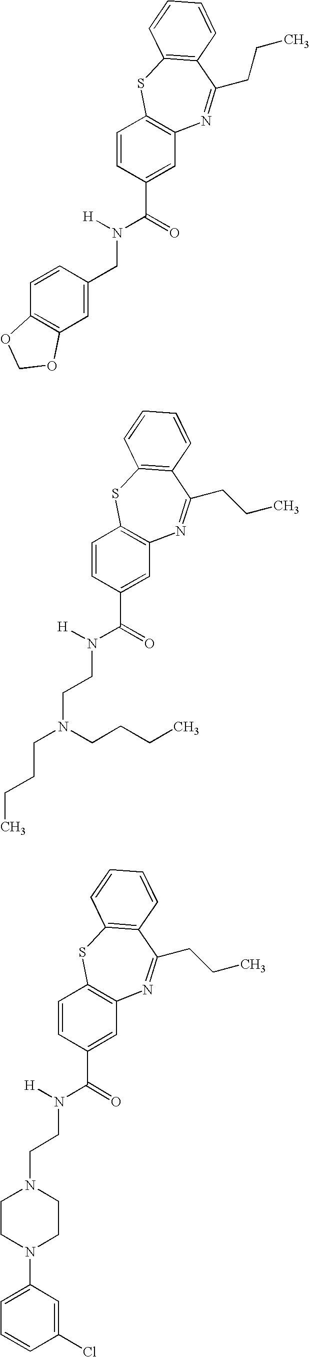

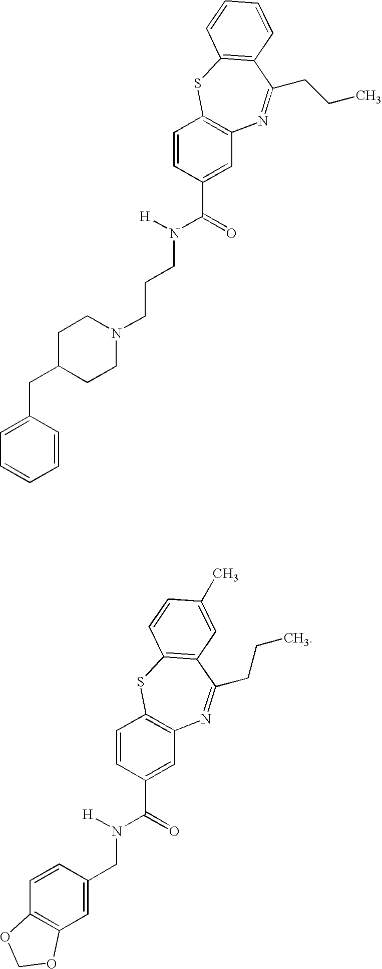

93. The compound of claim 1, wherein the compound is selected from the group consisting of:

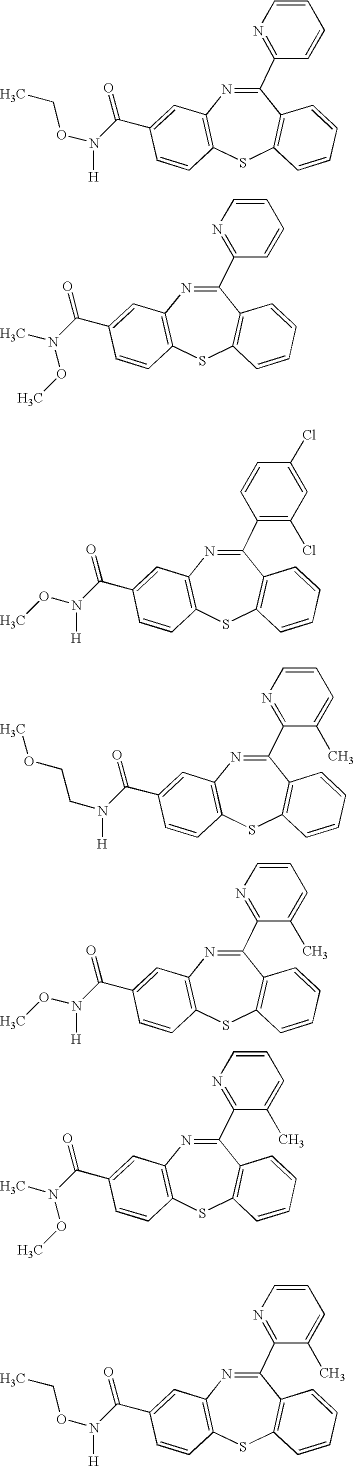

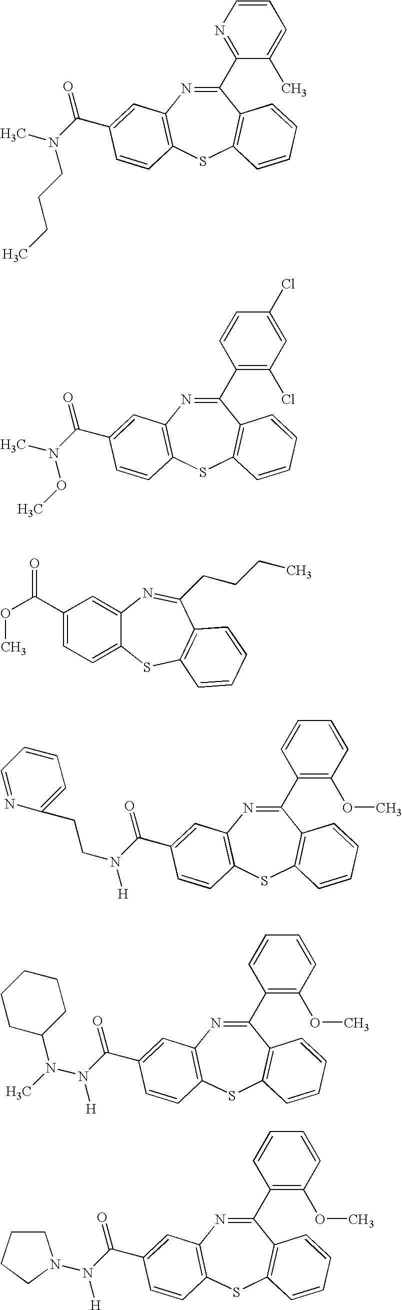

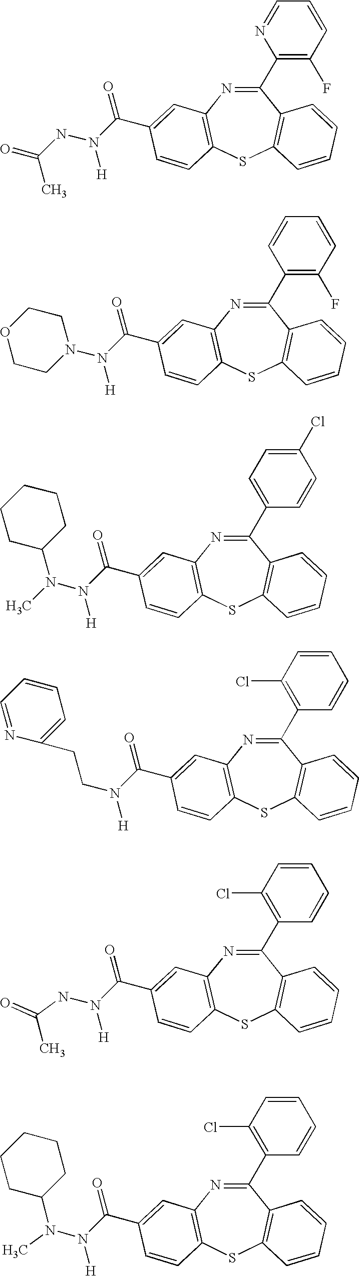

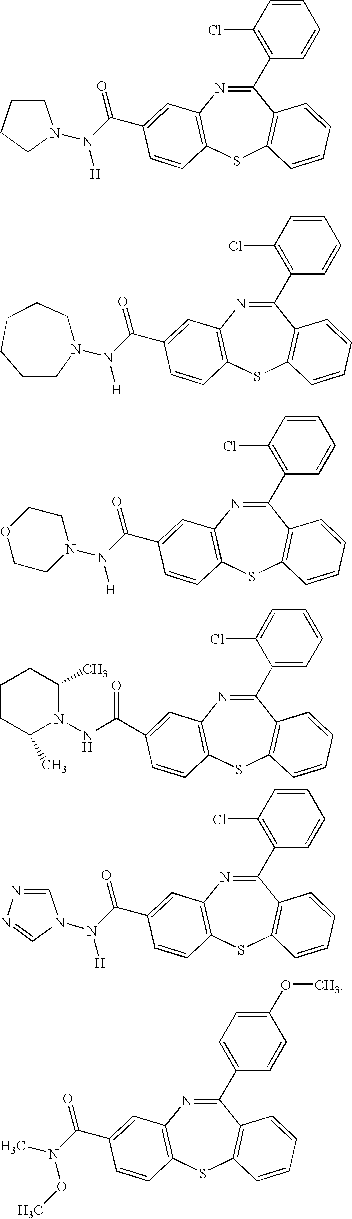

94. The compound of claim 1, wherein the compound is selected from the group consisting of:

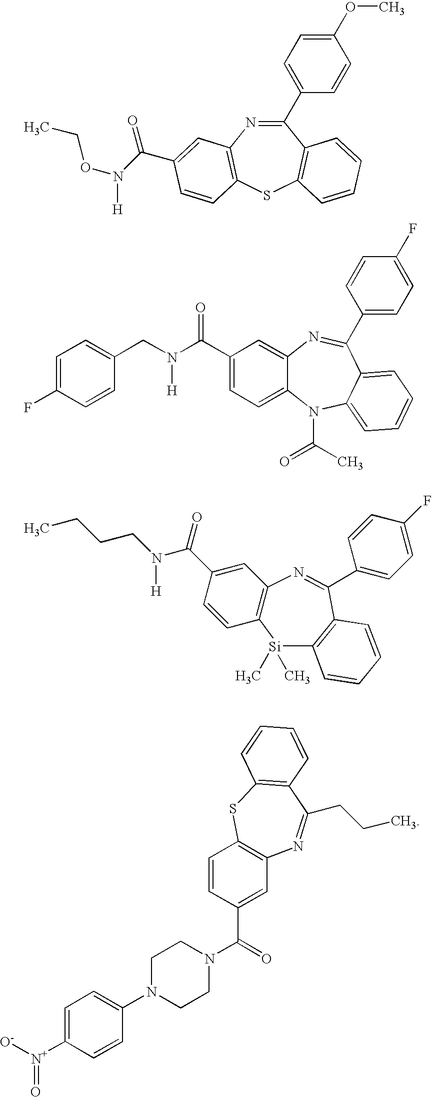

95. The compound of claim 1, wherein the compound is selected from the group consisting of:

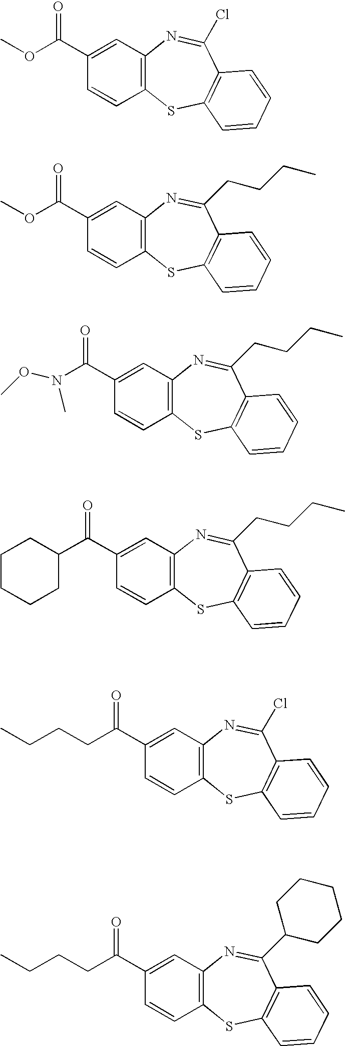

96. The compound of claim 1, wherein the compound is selected from the group consisting of:

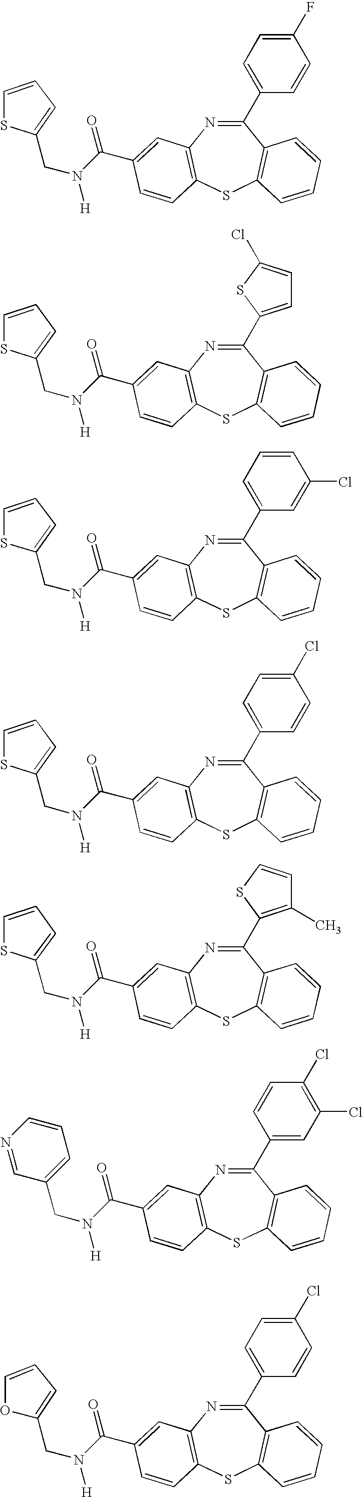

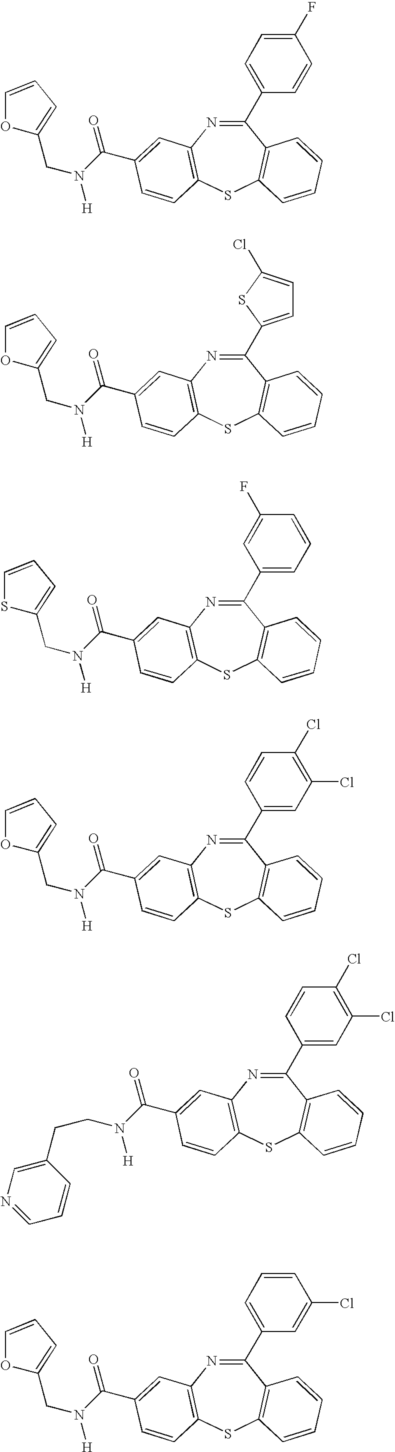

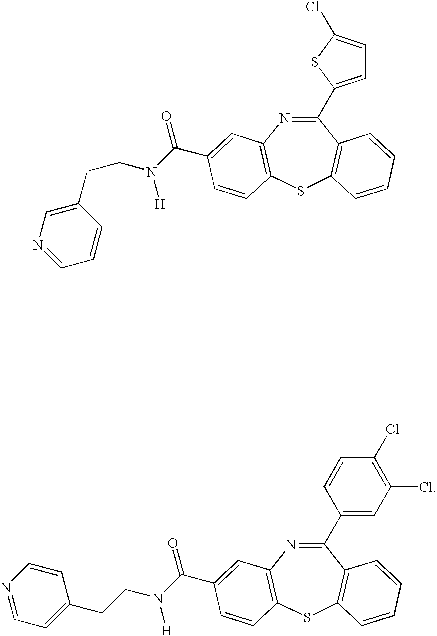

97. The compound of claim 1, wherein the compound is selected from the group consisting of:

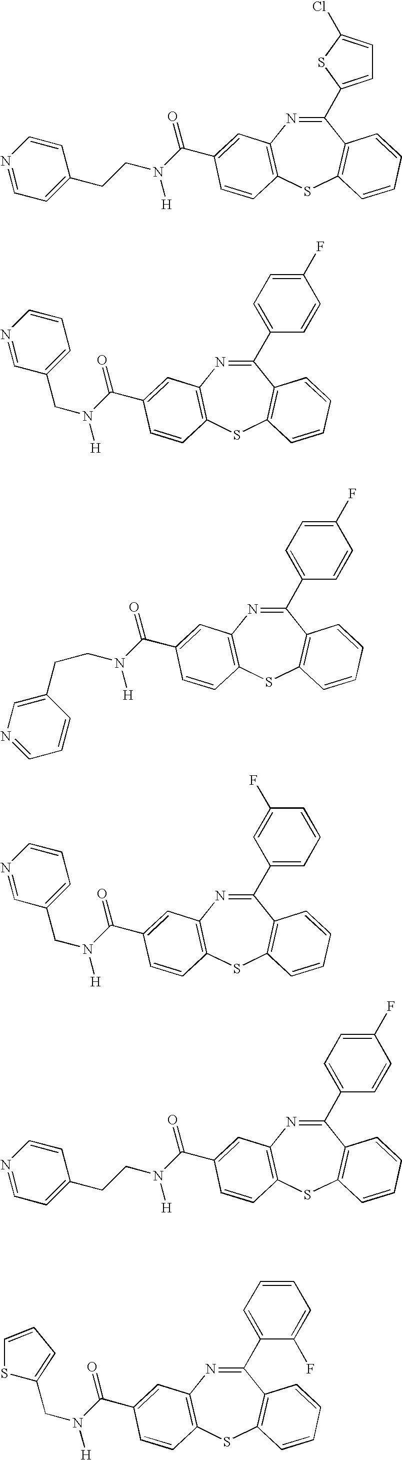

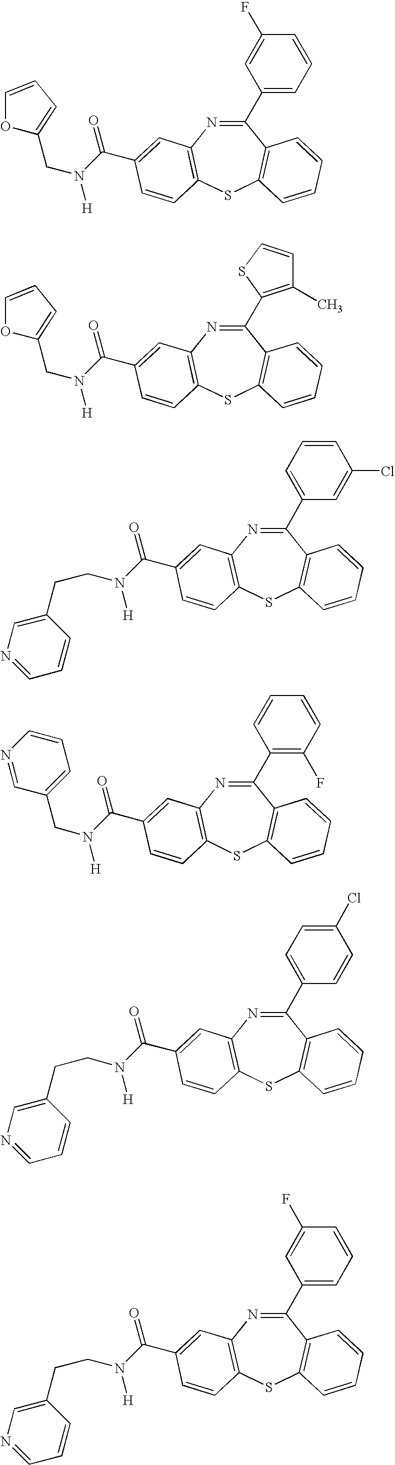

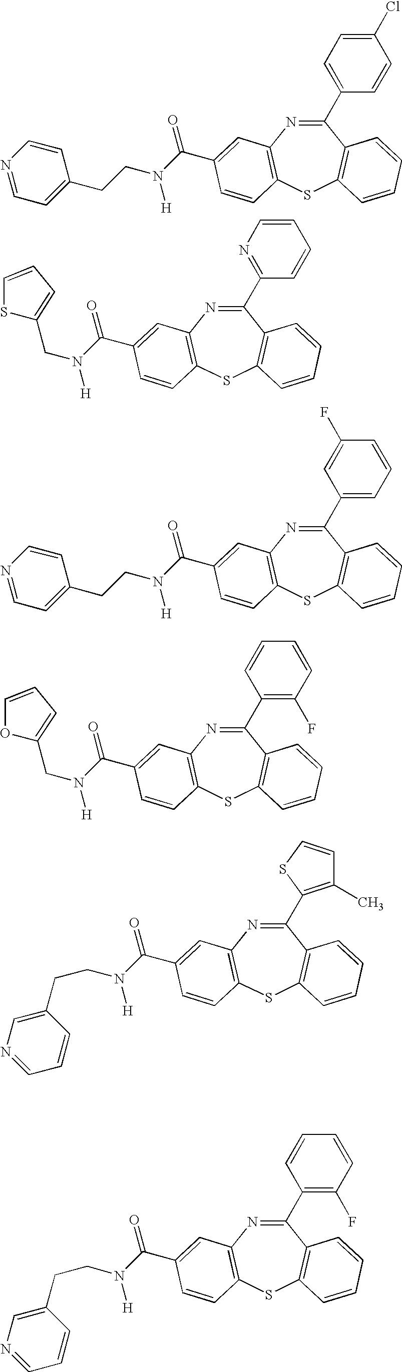

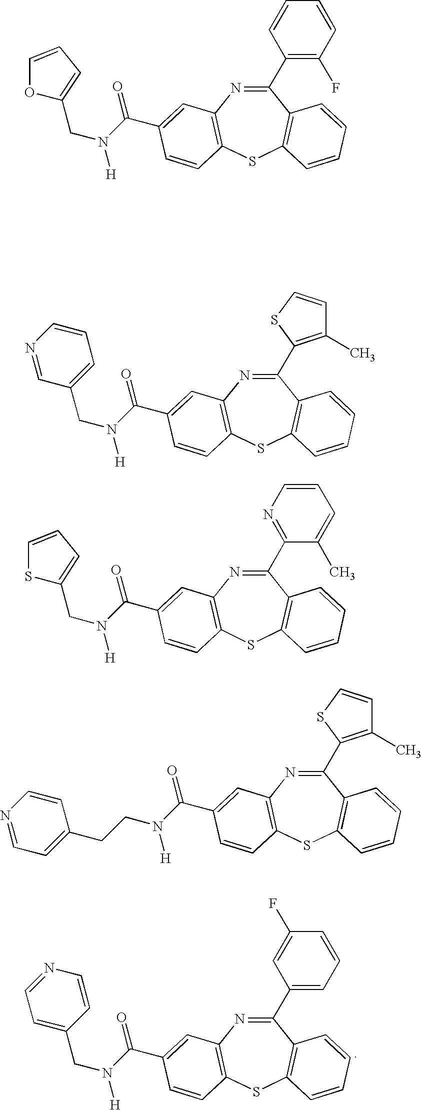

98. The compound of claim 1, wherein the compound is selected from the group consisting of:

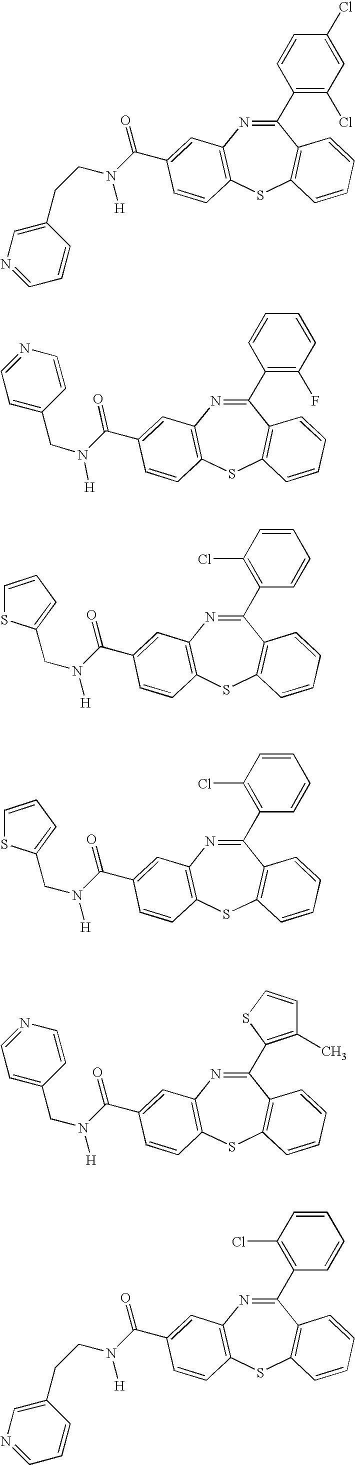

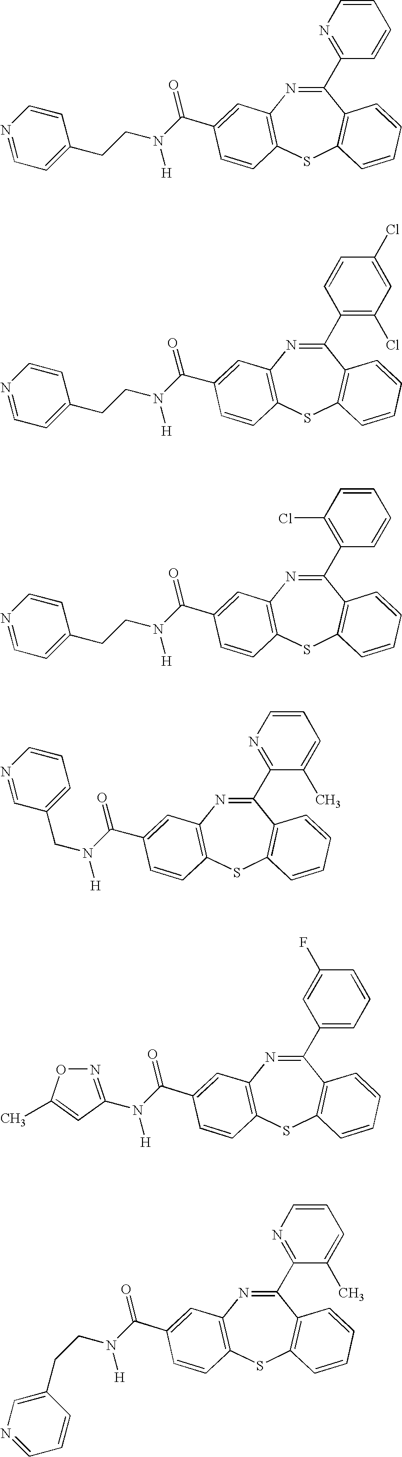

99. The compound of claim 1, wherein the compound is selected from the group consisting of:

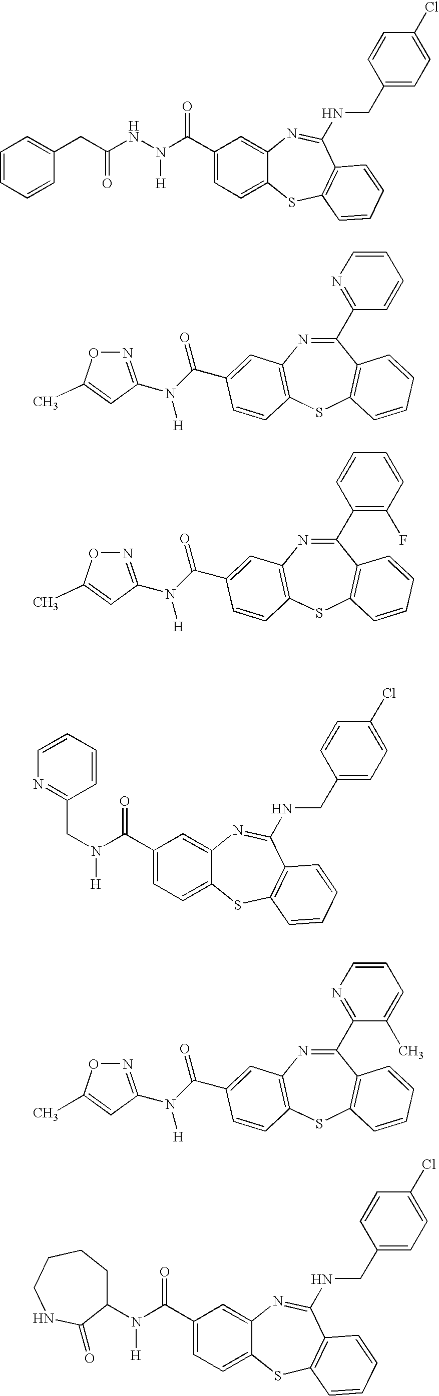

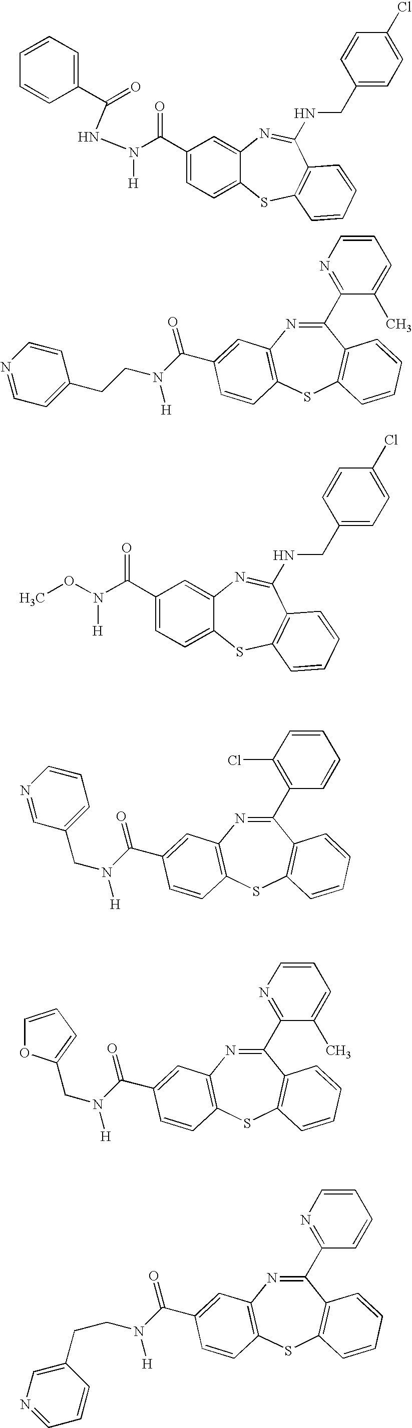

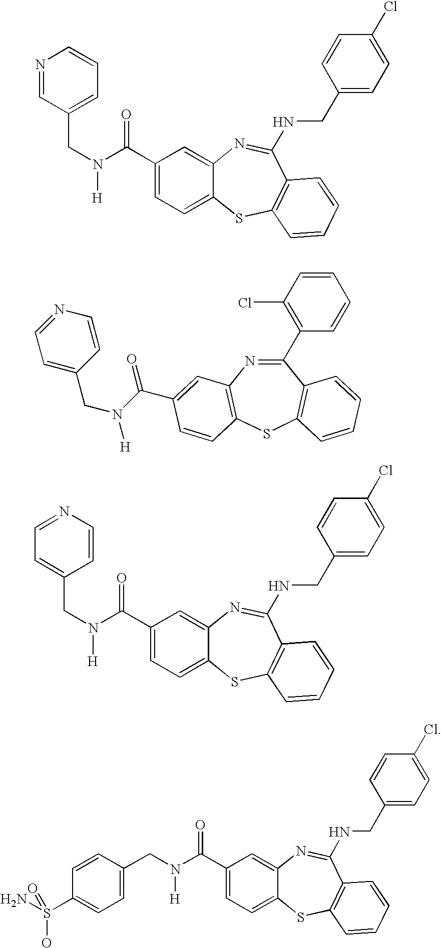

100. A pharmaceutical composition, comprising a therapeutically effective amount of a compound of claim 1 and a pharmaceutically acceptable carrier, diluent, or excipient.

101. A method of ameliorating or preventing a disease or condition selected from the group consisting of obesity, metabolic syndrome, a metabolic disorder, hypertension, polycystic ovary disease, osteoarthritis, a dermatological disorder, hypertension, insulin resistance, hypercholesterolemia, hypertriglyceridemia, cholelithiasis, a sleep disorder, hyperlipidemic conditions, bulimia nervosa, a compulsive eating disorder, an appetite disorder, atherosclerosis, diabetes, high cholesterol, hyperlipidemia, cachexia, an inflammatory disease, rheumatoid arthritis, a neurological disorder, a psychiatric disorder, substance abuse, depression, anxiety, mania, schizophrenia, dementia, dystonia, muscle spasticity, tremor, psychosis, an attention deficit disorder, a memory disorder, a cognitive disorder, short term memory loss, memory impairment, drug addiction, alcohol addiction, nicotine addiction, infertility, hemorrhagic shock, septic shock, cirrhosis, a cardiovascular disorder, cardiac dysfunction, valvular disease, myocardial infarction, cardiac hypertrophy, congestive heart failure, transplant rejection, an intestinal disorder, a neurodegenerative disease, multiple sclerosis, Alzheimer's disease, Parkinson's disease, epilepsy, Huntington's disease, Tourette's syndrome, cerebral ischaemia, cerebral apoplexy, craniocerebral trauma, stroke, spinal cord injury, catabolism, hypotension, hemorrhagic hypotension, endotoxin-induced hypotension, an eye disorder, glaucoma, uveitis, retinopathy, dry eye, macular degeneration, emesis, nausea, a gastric ulcer, diarrhea, pain, a neuropathic pain disorder, viral encephalitis, plaque sclerosis, cancer, a bone disorder, bone density loss, a lung disorder, asthma, pleurisy, polycystic ovary disease, premature abortion; inflammatory bowel disease, lupus, graft vs. host disease, T-cell mediated hypersensitivity disease, Hashimoto's thyroiditis, Guillain-Barre syndrome, contact dermatitis, allergic rhinitis, ischemic injury, and reperfusion injury comprising administering to the subject a therapeutically effective amount of a compound of claim 1.

102. The method of claim 101, wherein the therapeutically effective amount of a compound of claim 1 is in a sufficient amount to ameliorate or prevent said disease or condition by binding to a cannabinoid receptor.

103. The method of claim 101, further comprising identifying a subject in need of ameliorating or preventing said disease or condition.

104. The method of claim 101, wherein the neurological disorder is selected from the group consisting of schizophrenia, dementia, dystonia, muscle spasticity, tremor, psychosis, anxiety, depression, an attention deficit disorder, a memory disorder, a cognitive disorder, drug addiction, alcohol addiction, nicotine addiction, a neurodegenerative disease, multiple sclerosis, Alzheimer's disease, Parkinson's disease, epilepsy, Huntington's disease, Tourette's syndrome, cerebral ischaemia, cerebral apoplexy, craniocerebral trauma, stroke, spinal cord injury, pain, neuropathic pain disorder, viral encephalitis, and plaque sclerosis.

105. (canceled)

106. (canceled)

107. (canceled)

108. (canceled)

109. (canceled)

110. (canceled)

111. (canceled)

112. (canceled)

113. (canceled)

114. A method for treating or preventing a disease or condition in which it would be beneficial to modulate the activity of a CB1 receptor comprising administering a therapeutically effective amount of a compound of claim 1.

115. (canceled)

116. (canceled)

117. (canceled)

118. (canceled)

119. (canceled)

120. (canceled)

121. A method of improving cognition or memory in a subject comprising administering to a subject a pharmaceutically effective amount of a compound of claim 1.

122. (canceled)

123. (canceled)

124. (canceled)

125. A method of modulating or specifically inverse agonizing or antagonizing a cannabinoid receptor in a subject comprising administering to the subject an effective amount of a compound of claim 1.

126. The method of claim 125, wherein the cannabinoid receptor is a CB1 receptor.

127. A method of modulating or specifically inverse agonizing or antagonizing a cannabinoid receptor comprising contacting a cannabinoid receptor with a compound of claim 1.

128. The method of claim 127, wherein the cannabinoid receptor is a CB1 receptor.

129. A method of identifying a compound which is an agonist, inverse agonist, or antagonist of a cannabinoid receptor comprising:
contacting a cannabinoid receptor with at least one test compound of claim 1; and
determining any increase or decrease in activity level of the cannabinoid receptor so as to identify said test compound as an agonist, inverse agonist or antagonist of the cannabinoid receptor.

130. The method of claim 129, wherein the cannabinoid receptor is a CB1 receptor.

131. The method of claim 129, wherein said cannabinoid receptor consists essentially of SEQ ID NO: 2.

132. The method of claim 129, wherein said cannabinoid receptor has at least 90% amino acid identity to SEQ ID NO: 2.

133. The method of claim 129, wherein said cannabinoid receptor has at least 85% amino acid identity to SEQ ID NO: 2.

134. The method of claim 129, wherein said cannabinoid receptor has at least 70% amino acid identity to SEQ ID NO: 2.

135. A method of identifying a compound which is an agonist, inverse agonist, or antagonist of a cannabinoid receptor comprising:
culturing cells that express said a cannabinoid receptor;
incubating the cells or a component extracted from the cells with at least one test compound of claim 1; and
determining any increase or decrease in activity of the cannabinoid receptor so as to identify said test compound as an agonist, inverse agonist, or antagonist of the cannabinoid receptor.

136. (canceled)

137. (canceled)

138. (canceled)

139. (canceled)

140. (canceled)

141. A method for identifying a compound which binds to a cannabinoid receptor comprising:
labeling a compound of claim 1 with a detectable label; and
determining the number of occupied cannabinoid receptors.

142. The method of claim 141, wherein the detectable label is a radiolabel.

143. The method of claim 141, wherein the radiolabel is [³H].


SEQUENCE LISTING (4 sequences)

SEQ ID NO: 1 - DNA, 1254 bp, Homo sapiens
  CDS (1)...(1254): CB1 receptor
atgaagtcgatcctagatggccttgcagataccaccttccgcaccatcaccactgacctcctgtacgtgggctcaaatgacattcagtacgaagacatcaaaggtgacatggcatccaaattagggtacttcccacagaaattccctttaacttcctttaggggaagtcccttccaagagaagatgactgcgggagacaacccccagctagtcccagcagaccaggtgaacattacagaattttacaacaagtctctctcgtccttcaaggagaatgaggagaacatccagtgtggggagaacttcatggacatagagtgtttcatggtcctgaaccccagccagcagctggccattgcagtcctgtccctcacgctgggcaccttcacggtcctggagaacctcctggtgctgtgcgtcatcctccactcccgcagcctccgctgcaggccttcctaccacttcatcggcagcctggcggtggcagacctcctggggagtgtcatttttgtctacagcttcattgacttccacgtgttccaccgcaaagatagccgcaacgtgtttctgttcaaactgggtggggtcacggcctccttcactgcctccgtgggcagcctgttcctcacagccatcgacaggtacatatccattcacaggcccctggcctataagaggattgtcaccaggcccaaggccgtggtggcgttttgcctgatgtggaccatagccattgtgatcgccgtgctgcctctcctgggctggaactgcgagaaactgcaatctgtttgctcagacattttcccacacattgatgaaacctacctgatgttctggatcggggtcaccagcgtactgcttctgttcatcgtgtatgcgtacatgtatattctctggaaggctcacagccacgccgtccgcatgattcagcgtggcacccagaagagcatcatcatccacacgtctgaggatgggaaggtacaggtgacccggccagaccaagcccgcatggacattaggttagccaagaccctggtcctgatcctggtggtgttgatcatctgctggggccctctgcttgcaatcatggtgtatgatgtctttgggaagatgaacaagctcattaagacggtgtttgcattctgcagtatgctctgcctgctgaactccaccgtgaaccccatcatctatgctctgaggagtaaggacctgcgacacgctttccggagcatgtttccctcttgtgaaggctag

SEQ ID NO: 2 - protein, 417 aa, Artificial Sequence
  Truncated CB1 protein
MKSILDGLADTTFRTITTDLLYVGSNDIQYEDIKGDMASKLGYFPQKFPLTSFRGSPFQEKMTAGDNPQLVPADQVNITEFYNKSLSSFKENEENIQCGENFMDIECFMVLNPSQQLAIAVLSLTLGTFTVLENLLVLCVILHSRSLRCRPSYHFIGSLAVADLLGSVIFVYSFIDFHVFHRKDSRNVFLFKLGGVTASFTASVGSLFLTAIDRYISIHRPLAYKRIVTRPKAVVAFCLMWTIAIVIAVLPLLGWNCEKLQSVCSDIFPHIDETYLMFWIGVTSVLLLFIVYAYMYILWKAHSHAVRMIQRGTQKSIIIHTSEDGKVQVTRPDQARMDIRLAKTLVLILVVLIICWGPLLAIMVYDVFGKMNKLIKTVFAFCSMLCLLNSTVNPIIYALRSKDLRHAFRSMFPSCEG

SEQ ID NO: 3 - DNA, 1776 bp, Homo sapiens
  CDS (127)...(1209): CB2 protein
caggtcctgggagaggacagaaaacaactggactcctcagcccccggcagctcccagtgcccagccacccacaacacaacccaaagccttctagacaagctcagtggaatctgaagggcccaccccatggaggaatgctgggtgacagagatagccaatggctccaaggatggcttggattccaaccctatgaaggattacatgatcctgagtggtccccagaagacagctgttgctgtgttgtgcactcttctgggcctgctaagtgccctggagaacgtggctgtgctctatctgatcctgtcctcccaccaactccgccggaagccctcatacctgttcattggcagcttggctggggctgacttcctggccagtgtggtctttgcatgcagctttgtgaatttccatgttttccatggtgtggattccaaggctgtcttcctgctgaagattggcagcgtgactatgaccttcacagcctctgtgggtagcctcctgctgaccgccattgaccgatacctctgcctgcgctatccaccttcctacaaagctctgctcacccgtggaagggcactggtgaccctgggcatcatgtgggtcctctcagcactagtctcctacctgcccctcatgggatggacttgctgtcccaggccctgctctgagcttttcccactgatccccaatgactacctgctgagctggctcctgttcatcgccttcctcttttccggaatcatctacacctatgggcatgttctctggaaggcccatcagcatgtggccagcttgtctggccaccaggacaggcaggtgccaggaatggcccgaatgaggctggatgtgaggttggccaagaccctagggctagtgttggctgtgctcctcatctgttggttcccagtgctggccctcatggcccacagcctggccactacgctcagtgaccaggtcaagaaggcctttgctttctgctccatgctgtgcctcatcaactccatggtcaaccctgtcatctatgctctacggagtggagagatccgctcctctgcccatcactgcctggctcactggaagaagtgtgtgaggggccttgggtcagaggcaaaagaagaagccccgagatcctcagtcaccgagacagaggctgatgggaaaatcactccgtggccagattccagagatctagacctctctgattgctgatgaggcctcttcccaatttaaacaactcaagtcagaaatcagttcactccctggaagagagagaggggtcttggcactctcttcttacttaaaccagtcccagacacctagacacggacccctttttgctgatgagtgttgggactgactcctggaagacagcctggccttgcccacctgcacacagtctgttggataggtagggccacgaggagtagccaggtaggcgagacacaaaaaggcctgggacagggtcagtacaagtcaggacaggcttcatgcctgcatcctccagagaccaccaggagccaaagcgagcctccaggcccagcaatgagggacttgggagaaatctgagaagaatgggttgttctcttgggaagtcagggtatcagatgggatggacatccaggtcttctctctgcctaattgtcaaggcctccttggctctggagctatgaaaggccccactttcaagtcacccttgccactgaggaccgaggactatgctatgatgaggattaaggtgttgacttgcctctttcagagataaatgacaagccttca

SEQ ID NO: 4 - protein, 360 aa, Homo sapiens
MEECWVTEIANGSKDGLDSNPMKDYMILSGPQKTAVAVLCTLLGLLSALENVAVLYLILSSHQLRRKPSYLFIGSLAGADFLASVVFACSFVNFHVFHGVDSKAVFLLKIGSVTMTFTASVGSLLLTAIDRYLCLRYPPSYKALLTRGRALVTLGIMWVLSALVSYLPLMGWTCCPRPCSELFPLIPNDYLLSWLLFIAFLFSGIIYTYGHVLWKAHQHVASLSGHQDRQVPGMARMRLDVRLAKTLGLVLAVLLICWFPVLALMAHSLATTLSDQVKKAFAFCSMLCLINSMVNPVIYALRSGEIRSSAHHCLAHWKKCVRGLGSEAKEEAPRSSVTETEADGKITPWPDSRDLDLSDC